(19) United States
(12) Patent Application Publication
(10) Pub. No.: US 2024/0209078 A1
(43) Pub. Date: Jun. 27, 2024

(54) MULTITARGETING BISPECIFIC ANTIGEN-BINDING MOLECULES OF INCREASED SELECTIVITY

(71) Applicants: AMGEN INC., Thousand Oaks, CA (US); AMGEN RESEARCH (MUNICH) GMBH, Munich (DE)

(72) Inventors: Stephanie Everts, Munich (DE); Matthias Klinger, Munich (DE); Virginie Naegele, Munich (DE); Adam Zalewski, Munich (DE); Claudia Blümel, Munich (DE); Thomas Boehm, Munich (DE); Johannes Brozy, Munich (DE); Igor D'Angelo, Thousand Oaks, CA (US); Peter Kufer, Munich (DE); Petra Lutterbüse, Munich (DE); Markus Muenz, Munich (DE); Doris Rau, Munich (DE); Tobias Raum, Munich (DE); Benno Rattel, Munich (DE); Oliver Thomas, Munich (DE); Ines Ullrich, Munich (DE); Joachim Wahl, Munich (DE); Christian Webhofer, Munich (DE); Sascha Weidler, Munich (DE); Elizabeth Dang Pham, Thousand Oaks, CA (US); Julie Bailis, Thousand Oaks, CA (US)

(21) Appl. No.: 18/251,196

(22) PCT Filed: Nov. 8, 2021
(86) PCT No.: PCT/EP2021/080956

§ 371 Date: Apr. 28, 2023

Related U.S. Application Data
(60) Provisional application No. 63/231,877, filed on Aug. 11, 2021.
(60) Provisional application No. 63/110,957, filed on Nov. 6, 2020.

(51) Int. Cl. C07K 16/28; A61P 35/00; A61K 39/00

(52) U.S. Cl. CPC C07K 16/28; A61P 35/00; A61K 2039/505; C07K 2317/31; C07K 2317/524; C07K 2317/526; C07K 2317/53; C07K 2317/565; C07K 2317/92

(57) ABSTRACT

The present invention provides multitargeting bispecific antigen-binding molecules characterized by comprising a first and a second bispecific entity each comprising a domain binding to target, a second domain binding to an extracellular epitope of the human and the Macaca CD3ε chain, wherein both bispecific entities are linked to each other by a spacer which spaces apart the first and the second bispecific entity. Moreover, the invention provides a polynucleotide, encoding the multitargeting bispecific antigen-binding molecule, a vector comprising this polynucleotide, host cells, expressing the construct and a pharmaceutical composition comprising the same.

### TECHNICAL FIELD

This invention relates to products and methods of biotechnology, in particular to multitargeting antigen-binding molecules, their preparation and their use.

### BACKGROUND

The redirection of T cell activity against tumor cells by means of bispecific molecules independent of T cell receptor specificity is an evolving approach in immunooncology (Frankel S R, Baeuerle P A. Targeting T cells to tumor cells using bispecific antibodies. Curr Opin Chem Biol 2013; 17:385-92). Such new protein-based pharmaceuticals typically can simultaneously bind to two different types of antigen. They are known in several structural formats, and current applications have been explored for cancer immunotherapy and drug delivery (Fan, Gaowei; Wang, Zujian; Hao, Mingju; Li, Jinming (2015). “Bispecific antibodies and their applications”. Journal of Hematology & Oncology. 8: 130).

Bispecific molecules useful in immunooncology can be antigen-binding polypeptides such as antibodies, e.g. IgG-like, i.e. full-length bispecific antibodies, or non-IgG-like bispecific antibodies, which are not full-length antigen-binding molecules. Full length bispecific antibodies typically retain the traditional monoclonal antibody (mAb) structure of two Fab arms and one Fc region, except the two Fab sites bind different antigens. Non-full-length bispecific antibodies can lack an Fc region entirely. These include chemically linked Fabs, consisting of only the Fab regions, and various types of bivalent and trivalent single-chain variable fragments (scFvs). There are also fusion proteins mimicking the variable domains of two antibodies. An example of such a format is the bi-specific T-cell engager (BiTE®) (Yang, Fa; Wen, Weihong; Qin, Weijun (2016). “Bispecific Antibodies as a Development Platform for New Concepts and Treatment Strategies”. International Journal of Molecular Sciences. 18 (1): 48).

Exemplary bispecific antibody-derived molecules such as BiTE® molecules are recombinant protein constructs made from two flexibly linked antibody derived binding domains. One binding domain of BiTE® antigen-binding molecules is specific for a selected tumor-associated surface antigen on target cells; the second binding domain is specific for CD3, a subunit of the T cell receptor complex on T cells. By their particular design, BiTE® antigen-binding molecules are uniquely suited to transiently connect T cells with target cells and, at the same time, potently activate the inherent cytolytic potential of T cells against target cells. An important further development of the first generation of BiTE® antigen-binding molecules (see WO 99/54440 and WO 2005/040220) developed into the clinic as AMG 103 and AMG 110 was the provision of bispecific antigen-binding molecules binding to a context independent epitope at the N-terminus of the CD3ε chain (WO 2008/119567). BiTE® antigen-binding molecules binding to this elected epitope do not only show cross-species specificity for the human and the Macaca, or Callithrix jacchus, Saguinus oedipus or Saimiri sciureus CD3ε chain, but also, due to recognizing this specific epitope (instead of previously described epitopes of CD3 binders in bispecific T cell engaging molecules), do not demonstrate unspecific activation of T cells to the same degree as observed for the previous generation of T cell engaging antibodies. This reduction in T cell activation was connected with less or reduced T cell redistribution in patients, the latter being identified as a risk for side effects, e.g. in pasotuximab.

Antibody-based molecules as described in WO 2008/119567 are characterized by rapid clearance from the body; thus, while they are able to reach most parts of the body rapidly, their in vivo applications may be limited by their brief persistence in vivo. On the other hand, their concentration in the body can be adapted and fine-tuned at short notice. Prolonged administration by continuous intravenous infusion is used to achieve therapeutic effects because of the short in vivo half-life of this small, single chain molecule. However, bispecific antigen-binding molecules are available which have more favorable pharmacokinetic properties, including a longer half-life as described in WO 2017/134140. An increased half-life is typically useful in in vivo applications of immunoglobulins, especially with respect to antibody fragments or constructs of small size, e.g. in the interest of patient compliance.

One challenging ongoing problem in antibody-based immunooncology is tumor escape. Such tumor escape happens when the immune system—even if triggered or directed by some antibody-based immune-therapeutics—is not capable enough to eradicate tumors, which carry accumulated genetic and epigenetic alterations and use several mechanisms to be the victorious of the immunoediting process (Keshavarz-Fathi, Mahsa; Rezaei, Nima (2019) “Vaccines for Cancer Immunotherapy”). Generally, four mechanisms interfering with effective antitumor immune responses are known: (1) defective tumor antigen processing or presentation, (2) lack of activating mechanisms, (3) inhibitory mechanisms and immunosuppressive state, and (4) resistant tumor cells. Especially with respect to the first mechanism, tumor antigens might be present in a new form due to the genetic instability, mutation of the tumor and escape from immune system. Epitope-negative tumor cells remain hidden and consequently resistant to the immune rejection. They have been developed following the elimination of epitope-positive tumor cells, similar to Darwin's theory of natural selection. In consequence, antibody-based immune-therapy directed against an antigen on tumor cells is rendered ineffective when such tumor cells no longer express a respective antigen due to tumor escape. Said antigen loss is understood herein as driving force for tumor escape and thus, used interchangeably. Accordingly, there is a need to provide improved antibody-based immunooncology which addresses the problem of antigen loss to effectively prevent tumor escape.

A probably even more pressing challenge to the broad utilization of immunooncology with respect to T-cell engaging bispecific molecules is the availability of suitable targets (Bacac et al., Clin Cancer Res; 22(13) Jul. 1, 2016). For example, solid tumor targets may be overexpressed on tumor cells but expressed at lower, yet significant levels on nonmalignant primary cells in critical tissues. In nature, according to Bacac et al, T cells can distinguish between high- and low-antigen expressing cells by means of relatively low-affinity T cell receptors (TCRs) that can still achieve high-avidity binding to target cells expressing sufficiently high levels of target antigen. T-cell engaging bispecific molecules that could facilitate the same, and thus maximize the window between killing of high- and low-target expressing cells, are thus highly desirable. One approach discussed in the art is the use of dual targeting of two antigens which may lead to improved target selectivity over normal tissues that express only one or low levels of both target antigens. This effect is thought to be dependent on the avidity component mediated by the concurrent binding of the bsAb to both antigens on the same cell. With respect to dual targeting as such, some multispecific monoclonal antibodies (mAb) or other immune constructs are known in the art. WO 2014/116846 teaches a multispecific binding protein comprising a first binding site that specifically binds to a target cell antigen, a second binding site that specifically binds to a cell surface receptor on an immune cell, and a third binding site that specifically binds to cell surface modulator on the immune cell. US 2017/0022274 discloses a trivalent T-cell redirecting complex comprising a bispecific antibody, wherein the bispecific antibody has two binding sites against a tumor-associated antigen (TAA) and one binding site against a T-cell.

However, dual targeting alone as in molecules described above may not be sufficient for efficient target selectivity (Mazor et al, mAbs 7:3, 461-469; May/June 2015). Especially the configuration of the bsAb binding domains, namely monovalent vs. bivalent, is a critical factor. Even more, the provision of a bispecific molecule with several valences alone may not lead to clinically suitable therapeutic as also the potential risk profile in terms of significant immunological side effects such as cytokine release syndrome (CRS) has to be considered. Hence, despite the so-far achieved preclinical and clinical success of antibody-based immune-therapeutics, notable limitations remain including differential responses between individuals and cancer types. Not all patients will respond to therapy at available safe doses as dose-limiting toxicity can be a limiting factor for the efficacy of antibody-based immune-therapeutics. Hence, there is also a need to reduce dose-limiting toxicity in antibody-based immune-therapeutics to make such therapy available to more patients suffering from diverse proliferative diseases.

While different multispecific antibodies or antibody fragments are known in the art, some of which address T-cells, no multitargeting bispecific molecules have been proposed before which both addresses the need of overcoming dose-limiting toxicity in T cell redirecting immune-therapeutics by increasing the therapeutic window and are a stable and ready-to-use therapeutic system.

### SUMMARY

In view of the various unmet needs described above, it is an object of the present invention to provide a molecule which comprises at least one polypeptide chain which molecule is preferably an antigen-binding molecule. The molecule of the present invention is further preferably bispecific, such as a T cell engaging molecule. Further, the molecule of the present invention is preferably multitargeting, e.g. it is typically capable to immune-specifically bind to at least two antigens on a target cell which are typically associated with one or more diseases. It is further preferred that a molecule of the present invention is typically capable to immuno-specifically bind to two antigens on an effector cell at the same time, preferably for use in the treatment of said one or more diseases. Accordingly, the present invention provides a preferably multitargeting bispecific antigen-binding molecule comprising at least one polypeptide, wherein the molecule is characterized by comprising at least five distinctive structural entities, i.e. (i.) a first domain binding to a target cell surface antigen (e.g. a first tumor associated antigen, TAA), (ii.) a second domain binding to an extracellular epitope of the human (and preferably non-human primate, e.g. Macaca) CD3ε chain, wherein the first binding domain and the second binding domain together form a first bispecific entity, (iii.) a spacer which connects but also sufficiently spaces apart the first bispecific entity from a second bispecific entity comprising (iv.) a third domain binding to the same or preferably a different target cell surface antigen (e.g. a second TAA), and (v.) a fourth domain binding to an extracellular epitope of the human (and preferably non-human primate, e.g. Macaca) CD3ε chain. Preferably, the domains are comprised of VH and VL domains in amino to carboxyl orientation, respectively, wherein a flexible but short peptide linker links the VL of the first domain to the VH of the second domain and the VL of the third domain to the VH of the fourth domain, respectively. Surprisingly, a multitargeting bispecific antigen-binding molecule as described herein is typically capable to enable T-cells to distinguish between killing of cells expressing only one or both targets typically associated with a particular disease, thus opening a therapeutic window and reducing the risk for off-target toxicities and side effects. Moreover, the invention provides a polynucleotide encoding the multitargeting bispecific antigen-binding molecule, a vector comprising this polynucleotide, and host cells expressing the construct and a pharmaceutical composition comprising the same.

In a first aspect, it is envisaged in the context of the present invention to provide a molecule comprising at least one polypeptide chain, wherein the molecule comprises
(i.) a first binding domain, preferably comprising a paratope, which specifically binds to a first target cell surface antigen (e.g. TAA1),
(ii.) a second binding domain, preferably comprising a paratope, which specifically binds to an extracellular epitope of the human and/or the Macaca CD3ε chain,
(iii.) a third binding domain, preferably comprising a paratope, which specifically binds to a second target cell surface antigen (e.g. TAA2), and
(iv.) a fourth binding domain, preferably comprising a paratope, which specifically binds to an extracellular epitope of the human and/or the Macaca CD3ε chain,
wherein the first binding domain and the second binding domain form a first bispecific entity and the third and the fourth binding domain form a second bispecific entity, and
wherein the molecule comprises a spacer entity having a molecular weight of at least about 5 kDa and/or having a length of more than 50 amino acids, wherein the spacer entity spaces apart the first and the second bispecific entity by at least a distance of about 50 Å, wherein the indicated distance is understood as the distance between centers of mass of the first and the second bispecific entity, and which spacer entity is positioned between the first and the second bispecific entity.

Within said aspect, it is also envisaged in the context of the present invention to provide a molecule which is an antigen-binding molecule, preferably a bispecific antigen-binding molecule, more preferably a multitargeting bispecific antigen-binding molecule.

Within said aspect, it is also envisaged in the context of the present invention to provide an antigen-binding molecule, wherein the arrangement of domains in an amino to carboxyl order is selected from the group consisting of
(i.) first and second domain, spacer, third and fourth domain
(ii.) first and second domain, spacer, fourth and third domain
(iii.) second and the first domain, spacer, third and fourth domain, and
(iv.) second and first domain, spacer, fourth and third domain.

Within said aspect, it is also envisaged in the context of the present invention to provide an antigen-binding molecule, wherein said spacer entity has a molecular at least 10 kDa, more preferably at least 15 kDa, 20 kDa or even 50 kDa, and/or wherein said spacer entity comprises an amino acid sequence which comprises more than 50 amino acids, preferably at least 100 amino acids, more preferably at least 250 amino acids, and even more preferably at least 500 amino acids.

Within said aspect, it is also envisaged in the context of the present invention to provide an antigen-binding molecule, wherein said spacer entity is a rigid molecule which preferably folds into a secondary structure, preferably a helical structure, and/or a ternary structure, preferably a protein domain structure, most preferably a globular protein and/or parts thereof and/or combinations of globular proteins and/or parts thereof.

Within said aspect, it is also envisaged in the context of the present invention to provide an antigen-binding molecule, wherein the spacer entity is a globular protein, wherein the distance between the C alpha atoms of the first amino acid located at the N-terminus and the last amino acid at the C-terminus are spaced apart by at least 20 Å, preferably at least 30 Å, more preferably at least 50 Å, in order to effectively space apart the first and the second bispecific entity by preferably at least 50 Å.

Within said aspect, it is also envisaged in the context of the present invention to provide an antigen-binding molecule, wherein said spacer entity which effectively spaces apart the first and the second bispecific entity is selected from a group consisting of ubiquitin, beta 2 microglobulin, SAND domain, Green fluorescent protein (GFP), VHH antibody lama domain, PSI domain from Met-receptor, Fibronectin type III domain from tenascin, Granulocyte-macrophage colony-stimulating factor (GM-CSF), interleukin-4, CD137L Ectodomain, Interleukin-2, PD-1 binding domain from human Programmed cell death 1 ligand 1 (PDL1), Tim-3 (AS 24-130), MiniSOG, a programmed cell death protein 1 (PD1) domain, human serum albumin (HSA) or a derivate of any of the foregoing spacer entities, a multimer of a rigid linker, and a Fc domain or dimer or trimer thereof, each Fc domain comprising two polypeptide monomers comprising each a hinge, a CH2 and a CH3 domain a hinge and a further CH2 and a CH3 domain, wherein said two polypeptide monomers are fused to each other via a peptide linker or wherein the two polypeptide monomers are linked together by non-covalent CH3-CDH3 interactions and/or covalent disulfide bonds to form a heterodimer.

Within said aspect, it is also envisaged in the context of the present invention to provide an antigen-binding molecule, wherein said spacer entity is at least one Fc domain, preferably one domain or two or three covalently linked domains, which or each of which comprises in an amino to carboxyl order:
hinge-CH2-CH3-linker-hinge-CH2-CH3.

Within said aspect, it is also envisaged in the context of the present invention to provide an antigen-binding molecule, wherein each of said polypeptide monomers in the spacer entity has an amino acid sequence that is at least 90% identical to a sequence selected from the group consisting of: SEQ ID NO: 17-24, wherein preferably each of said polypeptide monomers has an amino acid sequence selected from SEQ ID NO: 17-24.

Within said aspect, it is also envisaged in the context of the present invention to provide an antigen-binding molecule, wherein the CH2 domain in the spacer comprises an intra domain cysteine disulfide bridge.

Within said aspect, it is also envisaged in the context of the present invention to provide an antigen-binding molecule, wherein the molecule is a single polypeptide chain.

Within said aspect, it is also envisaged in the context of the present invention to provide an antigen-binding molecule, wherein the spacer entity comprises an amino acid sequence selected the group consisting of SEQ ID NO: 13 and 15 to 16 and 25 to 34, ubiquitin (SEQ ID NO: 1081), beta 2 microglobulin (SEQ ID NO: 1083), SAND domain (SEQ ID NO: 1084), Green fluorescent protein (GFP) (SEQ ID NO: 1085), VHH antibody lama domain (SEQ ID NO: 1086), PSI domain from Met-receptor (SEQ ID NO: 1087), Fibronectin type III domain from tenascin (SEQ ID NO: 1088), Granulocyte-macrophage colony-stimulating factor (GM-CSF) (SEQ ID NO: 1089), interleukin-4 (SEQ ID NO: 1090), CD137L Ectodomain (SEQ ID NO: 1091), Interleukin-2 (SEQ ID NO: 1092), PD-1 binding domain from human Programmed cell death 1 ligand 1 (PDL1) (SEQ ID NO: 1093), Tim-3 (AS 24-130) (SEQ ID NO: 1094), MiniSOG (SEQ ID NO: 1095), a programmed cell death protein 1 (PD1) domain (SEQ ID NO: 16), human serum albumin (has, SEQ ID NO: 15) or an amino acid with at least 90%, preferably 95% or even 98% sequence identity thereof, preferably scFc (SEQ ID NO: 25).

Within said aspect, it is also envisaged in the context of the present invention to provide an antigen-binding molecule, wherein the molecule comprises two polypeptide chains.

Within said aspect, it is also envisaged in the context of the present invention to provide an antigen-binding molecule comprising two polypeptide chains, wherein
(i.) the first polypeptide chain comprises the first domain, the second domain, and the first polypeptide monomer preferably comprising hinge, a CH2 and a CH3 domain, and
(ii.) wherein the second polypeptide chain comprises the third domain, the fourth domain, and the second polypeptide monomer preferably comprising hinge, a CH2 and a CH3 domain,
wherein the two polypeptide monomers form a heterodimer pairing the CH2 and the CH3 domains of the two peptide monomers, respectively, wherein the CH2 domain of the first peptide monomer is linked to the first or second domain of the first bispecific entity in C-terminal position of said entity, and wherein the CH3 domain of the second peptide monomer is linked to the third or fourth domain of the second bispecific entity in N-terminal position of said entity, i.e. the N-terminus of the second polypeptide chain is at the CH2 domain of the second polypeptide monomer and the C-terminus is at the third or fourth domain,
wherein preferably the first and second polypeptide monomer form a heterodimer, thereby connecting the first and the second polypeptide chain.

Within said aspect it is also envisaged that the first peptide monomer of the first peptide chain is SEQ ID NO 35 and the second peptide monomer of the second peptide chain is SEQ ID NO 36, wherein the two peptide monomers preferably form a heterodimer.

Within said aspect it is also envisaged that the antigen-binding molecule is characterized by
(i) the first and third domain comprise two antibody-derived variable domains and the second and the fourth domain comprises two antibody-derived variable domains;
(ii) the first and third domain comprise one antibody-derived variable domain and the second and the fourth domain comprises two antibody-derived variable domains;
(iii) the first and third domain comprise two antibody-derived variable domains and the second and the fourth domain comprises one antibody-derived variable domain; or
(iv) the first domain comprises one antibody-derived variable domain and the third domain comprises one antibody-derived variable domain.

Within said aspect, it is also envisaged in the context of the present invention to provide an antigen-binding molecule comprising two polypeptide chains, wherein
the first polypeptide chain comprises a VH of the first domain, a VH second domain, the first polypeptide monomer comprising preferably a hinge, a CH2 and a CH3 domain, a VH of the third domain, and a VH of the fourth domain; and
the second polypeptide chain comprises a VL of the first domain, a VL second domain, the first polypeptide monomer comprising preferably a hinge, a CH2 and a CH3 domain, a VL of the third domain, and a VL of the fourth domain,
wherein preferably the first and second polypeptide monomer form a heterodimer, thereby connecting the first and the second polypeptide chain.

Within said aspect, it is also envisaged in the context of the present invention to provide an antigen-binding molecule, wherein the first, second, third and fourth binding domain each comprise in an amino to carboxyl order a VH domain and a VL domain, wherein the VH and VL within each domain is connected by a peptide linker, preferably a flexible linker which comprises serine, glutamine and/or glycine as amino acid building blocks, preferably only serine (Ser, S) or glutamine (Gln, Q) and glycine (Gly, G), more preferably (G4S)n or (G4Q)n, even more preferably SEQ ID NO: 1 or 3.

Within said aspect, it is also envisaged in the context of the present invention to provide peptide linker, wherein the peptide linker comprises or consists of S(G4X)n and (G4X)n, wherein X is selected from the group consisting of Q, T, N, C, G, A, V, I, L, and M, and wherein n is an integer selected from integers 1 to 20, preferably wherein n is 1, 2, 3, 4, 5 or 6, preferably wherein X is Q, wherein preferably the peptide linker is (G4X)n, n is 3, and X is Q.

Within said aspect, it is also envisaged in the context of the present invention to provide an antigen-binding molecule, wherein the peptide linker between the first binding domain and the second binding domain and the third binding domain and the fourth binding domain is preferably a flexible linker which comprises serine, glutamine and/or glycine or glutamic acid, alanine and lysine as amino acid building blocks, preferably selected from the group consisting of SEQ ID NO: 1 to 4, 6 to 12 and 1125.

Within said aspect, it is also envisaged in the context of the present invention to provide an antigen-binding molecule, wherein the peptide linker between the first binding domain or the second binding domain and the spacer, and/or the third binding domain and the fourth binding domain and the spacer, respectively, is preferably a short linker rich in small and/or hydrophilic amino acids, preferably glycine and preferably SEQ ID NO: 5.

Within said aspect, it is also envisaged in the context of the present invention to provide an antigen-binding molecule, wherein any of the first target cell surface antigen and the second target cell surface antigen is selected from the group consisting of CS1, BCMA, CDH3, FLT3, CD123, CD20, CD22, EpCAM, MSLN and CLL1.

Within said aspect, it is also envisaged in the context of the present invention to provide an antigen-binding molecule, wherein the first target cell surface antigen and the second target cell surface antigen are not identical.

Within said aspect, it is also envisaged in the context of the present invention to provide an antigen-binding molecule, wherein the first target cell surface antigen and the second target cell surface antigen are identical.

Within said aspect, it is also envisaged in the context of the present invention to provide an antigen-binding molecule, wherein the first binding domains is capable of binding to the first target cell surface antigen and the third binding domain is capable of binding to the second target cell surface antigen simultaneously, preferably wherein the first target cell surface antigen and the second target cell surface antigen are on the same target cell.

Within said aspect, it is also envisaged in the context of the present invention to provide an antigen-binding molecule of claim 1, wherein the first target cell surface antigen and the second target cell surface antigen, respectively, are selected from the group consisting of CS1 and BCMA, BCMA and CS1, FLT3 and CD123, CD123 and FLT3, CD20 and CD22, CD22 and CD20, EpCAM and MSLN, MSLN and EpCAM, MSLN and CDH3, CDH3 and MSLN, FLT3 and CLL1, and CLL1 and FLT3.

Within said aspect, it is also envisaged in the context of the present invention to provide an antigen-binding molecule of claim 1, wherein the first target cell surface antigen and/or the second target cell surface antigen is human MSLN (selected from SEQ ID NOs: 1181, 1182 and 1183), and wherein the first and/or third binding domain of the antigen-binding molecule of the invention binds to human MSLN epitope cluster E1 (SEQ ID NO: 1175, aa 296-346 position according to Kabat) as determined by murine chimere sequence analysis as described herein, but preferably not to human MSLN epitope cluster E2 (SEQ ID NO: 1176, aa 247-384 position according to Kabat), E3 (SEQ ID NO: 1177, aa 385-453 position according to Kabat), E4 (SEQ ID NO: 1178, aa 454-501 position according to Kabat) and/or E5 (SEQ ID NO: 1179 aa 502-545 position according to Kabat).

Within said aspect, it is also envisaged in the context of the present invention to provide an antigen-binding molecule of claim 1, wherein the first target cell surface antigen and/or the second target cell surface antigen is human CDH3 (SEQ ID NOs: 1170), and wherein the first and/or third binding domain of the antigen-binding molecule of claim 1 binds to human CDH3 epitope cluster D2B (SEQ ID NO: 1171, aa 253-290 position according to Kabat), D2C (SEQ ID NO: 1172 aa 291-327 position according to Kabat), D3A (SEQ ID NO: 1173 aa 328-363 position according to Kabat) and D4B (SEQ ID NO: 1174, aa 476-511 position according to Kabat), preferably D4B (SEQ ID NO: 1174, aa 476-511 position according to Kabat), as determined by murine chimere sequence analysis as described herein.

Within said aspect, it is also envisaged in the context of the present invention to provide an antigen-binding molecule, wherein the second and the fourth binding domain (CD3 binding domains) both have (i.) an affinity lower than characterized by a KD value of about 1.2×10-8 M measured by surface plasmon resonance (SPR), or (ii.) an affinity characterized by a KD value of about 1.2×10-8 M measured by SPR.

Within said aspect, it is also envisaged in the context of the present invention to provide an antigen-binding molecule, wherein the second and the fourth binding domain (CD3 binding domains) have an affinity characterized by a KD value of about 1.0×10-7 to 5.0×10-6 M measured by SPR, preferably about 1.0 to 3.0×10-6 M, more preferably about 2.5×10-6 M measured by SPR.

Within said aspect, it is also envisaged in the context of the present invention to provide an antigen-binding molecule, wherein the second and the fourth binding domain (CD3 binding domains) have an affinity characterized by a KD value of about 1.0×10-7 to 5.0×10-6 M measured by SPR, preferably about 1.0 to 3.0×10-6 M, more preferably about 2.5×10-6 M measured by SPR.

Within said aspect, it is also envisaged in the context of the present invention to provide an antigen-binding molecule, wherein each of the second and the fourth binding domain (CD3 binding domains) individually has an at least about 10-fold, preferably at least about 50-fold or more preferably at least about 100-fold lower activity than one CD3 binding domain comprising a VH according to SEQ ID NO 43 and a VL according to SEQ ID NO 44 (i.e. in a mono targeting context in contrast to a dual targeting context).

Within said aspect, it is also envisaged in the context of the present invention to provide an antigen-binding molecule, wherein the second and the fourth domain are effector binding domains binding to CD3ε chain which comprise or consist of a VH region linked to a VL region, wherein
i) the VH region comprises:
   a CDR-H1 sequence of X1YAX2N, where X1 is K, V, S, G, R, T, or I; and X2 is M or I;
   a CDR-H2 sequence of RIRSKYNNYATYYADX1VKX2, where X1 is S or Q; and X2 is D, G, K, S, or E; and
   a CDR-H3 sequence of HX1NFGNSYX2SX3X4AY, where X1 is G, R, or A; X2 is I, L, V, or T; X3 is Y, W or F; and X4 is W, F or Y; and
ii) wherein the VL region comprises:
   a CDR-L1 sequence of X1SSTGAVTX2X3X4YX5N, where X1 is G, R, or A; X2 is S or T; X3 is G or S; X4 is N or Y; and X5 is P or A;
   a CDR-L2 sequence of X1TX2X3X4X5X6; where X1 is G or A; X2 is K, D, or N; X3 is F, M or K; X4 is L or R; X5 is A, P, or V; and X6 is P or S; and
   a CDR-L3 sequence of X1LWYSNX2WV, where X1 is V, A, or T; and X2 is R or L; and
iii) wherein one or more of CDR sequences of the VH region of i) and/or of the VL region of ii) comprise one amino acid substitution or a combination thereof selected from X24V and X24F in CDR-H1;
   D15, and X116A in CDR-H2;
   H1, X12E, F4, and N6 in CDR-H3; and
   X11L and W3 in CDR-L3.

Within said aspect, it is also envisaged in the context of the present invention to provide an antigen-binding molecule, wherein the second and the fourth binding domain comprise a VH region comprising CDR-H 1, CDR-H2 and CDR-H3 selected from SEQ ID NOs 37 to 39, 45 to 47, 53 to 55, 61 to 63, 69 to 71, 436 to 438, 1126 to 1128, 1136 to 1138, 1142 to 1144, and 1148 to 1150, and a VL region comprising CDR-L1, CDR-L2 and CDR-L3 selected from SEQ ID NOs 40 to 42, 48 to 50, 56 to 58, 64 to 66, 72 to 74, 439 to 441, 1129 to 1131, 1139 to 1141, 1145 to 1147, and 1151 to 1153, preferably 61 to 63 and 64 to 66.

Within said aspect, it is also envisaged in the context of the present invention to provide an antigen-binding molecule, wherein the second and fourth binding domain comprise a VH region selected from SEQ ID NOs 43, 51, 59, 67, 75, 442 and 1132, preferably 67.

Within said aspect, it is also envisaged in the context of the present invention to provide an antigen-binding molecule, wherein the second and fourth binding domain comprise a VL region selected from SEQ ID NOs 44, 52, 60, 68, 76, 443 and 1133, preferably 68.

Within said aspect, it is also envisaged in the context of the present invention to provide an antigen-binding molecule, wherein the second and fourth binding domain comprising a VH region selected from SEQ ID NOs 43, 51, 59, 67, 75, 442 and 1132, preferably 67, and a VL region selected from SEQ ID NOs 44, 52, 60, 68, 76, 443 and 1133, preferably 68, wherein when the VH region is 1132 and the VL region is 1133, the second and/or fourth binding domain as scFab domain additionally comprises a CH1 domain of SEQ ID NO: 1134 and a CLK domain of SEQ ID NO: 1135, and wherein the VH and VL region are linked to each other by a linker preferably selected from SEQ ID NO 1, 3 and 1125.

Within said aspect, it is also envisaged in the context of the present invention to provide an antigen-binding molecule, wherein the first and/or the third (target) binding domain bind to CDH3 and comprise a VH region comprising SEQ ID NO: 1154 as CDR-H 1 wherein X1 (the number behind the “X” indicates the numerical order of the “X” in respective amino acid sequence in N- to C-orientation in the sequence table) is S or N, X2 is Y or S, X3 is P or W, X4 is I or M and X5 is Y, N or H; SEQ ID NO: 1155 as CDR-H2 wherein X1 is K, V, N or R; X2 is A, D, R, Y, S, W or H; X3 is Y, S, P, G or T; X4 is S, G or K; X5 is A, V, D, K, G, or T; X6 is A, V, D, K, S, G or H; X7
is Y, G, or E; X8 is K, I, or N; X9 is A, S, or N; X10 is S, Q or G; X11
is S or K; X12 is F or V; and X13 is K or Q; and SEQ ID NO: 1156 as CDR-H3, wherein X1 is F or Q; X2 is R,K,S or W; X3 is G or D; X4 is Y, P or R; X5 is R, S, G, N or T; X6
is Y,A or H; X7 is F, L or M; X8 is A or V; and X9 is Y or V; and wherein the first and/or the third (target) binding domain bind to CDH3 and comprise a VL region comprising SEQ ID NO: 1158 as CDR-L 1 wherein X1 is K or R, X2 is A or S; X3 is Q,D,S,G or E; X4 is S,D or N; X5 is V,L or I; X6 is, K,Y,S, or H; X7 is S or N; X8 is F,L or M; and X9 is A,N or H; SEQ ID NO: 1159 as CDR-L 2 wherein X1 is Y,G,W,N; X2 is T or A; X3 is S or K; X4 is T,N or R; X5 is L or R; X6 is E,A,V or H; and X7 is S or E; and SEQ ID NO: 1160 as CDR-L3 wherein X1 is Q or V; X2 is Q,N or H; X3 is F,L,Y,W,N, or H; X4 is A,D,Y,S or N; X5 is Q,R,S,G,W or M; X6 is T,Y or F; and X7 is F,Y or L.

Within said aspect, it is also envisaged in the context of the present invention to provide an antigen-binding molecule, wherein the first and/or the third (target) binding domain bind to MSLN and comprise a VH region comprising SEQ ID NO: 1162 as CDR-H 1 wherein X1 (the number behind the “X” indicates the numerical order of the “X” in respective amino acid sequence in N- to C-orientation in the sequence table) is S,G or D; X2 is Y,A,G or F; X3 is I,W, or M; and X4 is V,S,G,T, or H; SEQ ID NO: 1163 as CDR-H 2 wherein X1 is A,S,N,W,Y, or V; X2 is Y,S or N; X3 is Y,G,P, or S; X4 is D,H,S, or N; X5 is G or S; X6 is E,G or S; X7 is G,S,N,F,T or Q; X8
is S,W,K,D,I or T; X9 is Y or N; X10 is A or N; X11 is A,P,N,D,E,I or Q; X12 is D,A,S or K; X13 is V,L, or F; X14 is K or Q; and X15 is G or S; and SEQ ID NO: 1164 as CDR-H 3 wherein X1 is D,E or V; X2 is R,G, or E; X3 is Y,A, or N; X4 is S,Y,V, or H; X5 is A,P,F,Y, or H; X6 is R or S; X7 is E or G; X8 is Y or L; X9 is R,Y or L; X10
is Y or G; X11 is D or Y; X12 is R,Y, or F; X13 is M,S,F,D or Y; X14 is A,G,S, or T; X15 is L, M, or F; and X16 is Y,I or V; and wherein the first and/or the third (target) binding domain bind to MSLN and comprise a VL region comprising SEQ ID NO: 1166 as CDR-L 1 wherein X1
is A or S; X2 is G or S; X3 is E or Q; X4 is G,S or K; X5 is I,L,V or F; X6 is R,G or S; X7 is D,S,N or T; X8 is A,S,K or T; X9 is Y or W; X10 is V or L; and X11 is Y or A; SEQ ID NO 1167 as CDR-L2 wherein X1 is A,G or Q; X2 is A or S; X3 is S or T; X4
is G,S,K,I or T; X5 is R or L; X6 is A,P or Q; and X7 is S or T; and SEQ ID NO 1168 as CDR-L 3 wherein X1 is A or Q; X2 is Y,S,A, or T; X3 is G,E,Y,H or Q; X4 is A or S; X5
is S,T or F; X6 is -,P or T; X7 is R,A,L or F; and X8 is V or T.

Within said aspect, it is also envisaged in the context of the present invention to provide an antigen-binding molecule, wherein the first and/or the third (target) binding domain bind to CDH3 and comprise a VH region of SEQ ID NO: 1157 wherein (the number behind the “X” indicates the numerical order of the “X” in respective amino acid sequence in N- to C-orientation in the sequence table) X1 is Q or E; X2 is V,L; X3 is Q,E; X4 is A or G; X5 is G or E; X6 is V or L; X7 is K or V X8 is K or Q, X9 is A or G, X10 is V or L, X11 is K or R, X12 is V or L, X13 is A or K, X14 Y or F, X15 is T or S, X16 is T or S, X17 is S or N, X18 is Y or S, X19 is P or W, X20 is is I or M, X21 is Y,N or H, X22 is Tor A, X23 is Q or K, X24 is V or M, X25 is S or G, X26 is K, V,N or R, X27 is A,D,R, Y,S, W or H, X28 is Y,S,P,Gr or T, X29 is S,K, or G, X30 is A,V,D,K, or, T, X31 is A,-,D,K,S,G, or H, X32 is Y,G, or E, X33 is K,I, or N, X34 is A,S, or N, X35 is S,Q, or G, X36 is Sor K, X37 is F or V, X38 is Q or K, X39 is F or V, X40 is I or M, X41 is T or S, X42 is V,I or R, X43 is T,K or N, X44 is T,A,S or K, X45 is S or N, X46 is A, V or L, X47 is L or M, X48 is Q or E, X49 is
L or M, X50 is S or N, X51 is S or R, X52 is T or R, X53 is A or S, X54 is G,D, or E, X55 is T or S, X56 is T,K, or R, X57 is S,Q, W, or R, X58 is -,D, or G, X59 is Y,P, or R, X60 is F,S,G,N or T, X61 is Y,A, or H, X62 is A,-, or V, X63 is F or M, X64 is Y or V; X65 is T,L or M; and a VL region of SEQ ID NO 1161 wherein X1 is D or E; X2 Q or V; X3 is L,M; X4 is A,S or D; X5 is F,S or T; X6 is A,S; X7 is A,V; X8 is
P,V,L; X9 is D,E; X10 is A,V; X11 is I,L; X12 is T,S,N; X13 is K,R; X14 is
A,S; X15 is Q,D,S,G or E; X16 is S,D,N; X17 is V,I or L; X18 is -,K,Y,S or H; X19 is S,N; X20 is F,L,M; X21 is A,N,H; X22 is K,Q; X23 is A,P,V; X24 is K,R; X25 is
I, V; X26 is Y,G,W,N; X27 is T,A; X28 is S,K; X29 is T,N,R; X30 is L,R; X31 is E,A,V,H; X32 is S,E; X33 is A,S,V,D; X34 is D,E; X35 is T,K; X36 is S,R; X37 is A,S,P; X38 is F,V; X39 is A,G; X40 is T,V; X41 is Q,V; X42 is Q,N,H; X43 is F,L, Y, W,N,H; X44 is A,D,Y,S,N; X45 is is F, Y,T; X47 is F,Y,L; X48 Q,R,S,G,W,M; X46
is V,L; and X49 is D or E (wherein all aa per position are meant to be in the alternative “or” even if not explicitly stated).

Within said aspect, it is also envisaged in the context of the present invention to provide an antigen-binding molecule, wherein the first and/or the third (target) binding domain bind to MSLN and comprise a VH region of SEQ ID NO: 1165 wherein (the number behind the “X” indicates the numerical order of the “X” in respective amino acid sequence in N- to C-orientation in the sequence table) X1 is E,Q; X2 is V,L,Q, X3 is E,Q; X4 is A,G,P; X5 is E,G; X6 is V,L; X7 is V,K; X8 is K,Q; X9 is G,S; X10 is E,A,G,R; X11 is S,T; X12
is V,L; X13 is R,S,K; X14 is V,L; X15 is S,T; X16 is A,K,T; X17 is A,V; X18
is Y,I,F; X19 is S,T; X20 is S,F; X21 is S,T; X22 is D,G,S; X23 is Y,G,A,F; X24 is I, W,M; X25 is G,S,V,T,H; X26 is I, V; X27 is A,P; X28 is M,K,Q; X29 is G,C; X30 is I,M, V,L; X31 is A,G,S; X32 is A,S,N,W, Y, V; X33 is Y,S,N; X34 is Y,G,P,S; X35 is D,H,S,N; X36 is G,S; X37 is E,G,S; X38 is G,S,N,F,T,Q; X39 is S,K,W,D,I,-,T; X40 is Y,N; X41 is A,N; X42 is A,P,N,E,D,I,Q; X43 is D,A,S,K; X44
is V,L,F; X45 is K,Q; X46 is G,S; X47 is V,F; X48 is I,M; X49 is S,T; X50
is R,V; X51 is N,T; X52 is A,S; X53 is I,K; X54 is S,N; X55 is S,T,Q; X56
is A,L,F; X57 is Y,S,F; X58 is L,M; X59 is E,K,Q; X60 is M,L; X61 is S,N; X62
is R,S; X63 is V,L; X64 is R,T; X65 is A,S; X66 is D,A,E; X67 is R,K; X68
is D,E, V,L; X69 is E,R,G,P; X70 is R,A,N, Y; X71 is G,S,Y,V,H; X72
is A,P,F,D,Y; X73 is R,G; X74 is M,R,S,D; X75 is E,G; X76 is Y,L; X77 is Y, F; X78 is Y,S,F; X79 is A,G,S,T,H; X80 is L,M,F; X81 is Y,I,V; and X82 is L,M,T; and a VL region of SEQ ID NO 1169 (the number behind the “X” indicates the numerical order of the “X” in respective amino acid sequence in N- to C-orientation in the sequence table) X1
is E,S,D; X2 is Y,I,L; X3 is E,-,V,T; X4 is V,L,M; X5 is P,S; X6 is G,S; X7
is S,T; X8 is V,L; X9 is A, V,L; X10 is P,V; X11 is E,Q,D; X12 is R,T; X13
is A, V; X14 is S,T; X15 is I,L; X16 is S,T; X17 is A,S; X18 is G,S; X19
is E,Q; X20 is G,S,K; X21 is I,V,L,F; X22 is R,G,S; X23 is D,S,-; X24 is A,S,N,K,T; X25 is Y, WM; X26 is V,L; X27 is Y,A; X28 is K,Q; X29 is A,S,V; X30 is R,V,K; X31
is V,L; X32 is A,G,Q; X33 is A,S; X34 is S,T; X35 is G,S,K,I,T; X36 is R,L; X37 is A,P,Q; X38 is S,T; X39 is I, V; X40 is E,S,D; X41 is G,N; X42 is N,T; X43 is D,T; X44 is A,F; X45 is R,G,S; X46 is L,T; X47 is E,Q; X48 is A,P; X49 is E,M; X50 is E,F; X51 is D, V,T; X52 is A,Q; X53 is Y,S,A,T; X54
is G,E, Y,H,Q; X55 is A,S; X56 is S,T,F; X57 is P,T; X58 is R,A,L,F; X59
is V,T; X60 is P,C; X61 is V,L; X62 is E,T; X63 is I, V; and X64 is L,K (wherein all aa per position are meant to be in the alternative “or” even if not explicitly stated).

Within said aspect, it is also envisaged in the context of the present invention to provide an antigen-binding molecule, wherein the first and/or the third (target) binding domain comprise a VH region comprising CDR-H 1, CDR-H2 and CDR-H3 selected from SEQ ID NO: 77 to 79, 86 to 88, 95 to 97, 103 to 105, 111 to 113, 119 to 121, 127 to 129, 135 to 137, 143 to 145, 151 to 153, 159 to 161, 168 to 170, 177 to 179, 185 to 187, 194 to 196, 203 to 205, 212 to 214, 221 to 223, 230 to 232, 238 to 240, 334 to 336, 356 to 358, 365 to 367, 376 to 378, 385 to 387, and 194, 432 and 196, or any combination of CDR-H 1, CDR-H2 and CDR-H3 as disclosed together in the sequence table Tab. 50, preferably 86 to 88 and 194, 432 and 196.

Within said aspect, it is also envisaged in the context of the present invention to provide an antigen-binding molecule, wherein the first and/or third (target) binding domain comprise a VL region comprising CDR-L1, CDR-L2 and CDR-L3 selected from SEQ ID NO: 80 to 82, 89 to 91, 98 to 100, 106 to 108, 114 to 116, 122 to 124, 130 to 132, 138 to 140, 146 to 148, 154 to 156, 162 to 164, 171 to 173, 180 to 182, 188 to 190, 197 to 199, 206 to 208, 215 to 217, 224 to 226, 233 to 235, 241 to 243, 337 to 339, 359 to 361, 368 to 370, 379 to 381, 388 to 390, preferably 89 to 91 and 197 to 199.

Within said aspect, it is also envisaged in the context of the present invention to provide an antigen-binding molecule, wherein the first and/or third (target) binding domain comprise a VH region selected from SEQ ID NO: 83, 92, 101, 109, 117, 125, 133, 141, 149, 157, 165, 174, 183, 191, 200, 209, 218, 227, 236, 244, 340, 362, 371, 382, 391 and 433, preferably 433 and 92 and for the first and third binding domain, respectively.

Within said aspect, it is also envisaged in the context of the present invention to provide an antigen-binding molecule, wherein the first and/or third (target) binding domain comprises a VL region selected from SEQ ID NO: 84, 93, 102, 110, 118, 126, 134, 142, 150, 158, 166, 175, 184, 192, 201, 210, 219, 228, 237, 245, 341, 363, 372, 383, 392, preferably 200 and 93 for the first and third binding domain, respectively.

Within said aspect, it is also envisaged in the context of the present invention to provide an antigen-binding molecule, wherein the first and/or third (target) binding domain comprises a VL region of increased stability by a single amino acid exchange (E to I), selected from SEQ ID NO: 85, 94, 193, 202, 211, 220, 229, 364, 384, 393, preferably 94 and 202.

Within said aspect, it is also envisaged in the context of the present invention to provide an antigen-binding molecule, having an amino acid sequence selected from the group consisting of SEQ ID NOs: 246 to 323 or 330 to 332, 351 to 355, 373 to 375, 394 to 410 and 434, preferably 434.

In a second aspect, it is further envisaged in the context of the present invention to provide a polynucleotide encoding an antigen-binding molecule of the present invention, preferably selected from SEQ ID NO: 1070 to 1072 and 1074.

In a third aspect, it is also envisaged in the context of the present invention to provide a vector comprising a polynucleotide of the present invention.

In a fourth aspect, it is further envisaged in the context of the present invention to provide a host cell transformed or transfected with the polynucleotide or with the vector of the present invention.

In a fifth aspect, it is also envisaged in the context of the present invention to provide a process for the production of an antigen-binding molecule of the present invention, said process comprising culturing a host cell of the present invention under conditions allowing the expression of the antigen-binding molecule and recovering the produced antigen-binding molecule from the culture.

In a sixth aspect, it is further envisaged in the context of the present invention to provide a pharmaceutical composition comprising an antigen-binding molecule of the present invention or produced according to the process of the present invention.

Within said aspect, is also envisaged in the context of the present invention that the pharmaceutical composition is stable for at least four weeks at about −20° ° C.

It is further envisaged in the context of the present invention to provide the antigen-binding molecule of the present invention, or produced according to the process of the present invention, for use in the prevention, treatment or amelioration of a disease selected from a proliferative disease, a tumorous disease, cancer or an immunological disorder.

Within said aspect, it is also envisaged in the context of the present invention that the disease preferably is acute myeloid leukemia (AML), Non-Hodgkin lymphoma (NHL), Non-small-cell lung carcinoma (NSCLC), pancreatic cancer and Colorectal cancer (CRC)]. In a seventh aspect, it is further envisaged in the context of the present invention to provide a method for the treatment or amelioration of a proliferative disease, the method comprising administering to a subject in need thereof a molecule comprising at least one polypeptide chain, wherein the molecule comprises
(i.) a first binding domain which preferably comprises a paratope which specifically binds to a first target cell surface antigen (e.g. TAA1),
(ii.) a second binding domain which preferably comprises a paratope which specifically binds to an extracellular epitope of the human—and preferably the Macaca—CD3ε chain,
(iii.) a third binding domain which preferably comprises a paratope which specifically binds to a second target cell surface antigen (e.g. TAA2), and
(iv.) a fourth binding domain which preferably comprises a paratope which specifically binds to an extracellular epitope of the human—and preferably the Macaca—CD3ε chain,
wherein the first binding domain and the second binding domain form a first bispecific entity and the third and the fourth binding domain form a second bispecific entity, and
wherein the molecule comprises a spacer entity having a molecular weight of at least about larger than about 5 kDa and/or having a length of more than 50 amino acids, wherein the spacer entity spaces apart the first and the second bispecific entity by at least about 50 Å (distance between centers of mass of the first and the second bispecific entity), and which spacer entity is positioned between the first and the second bispecific entity.

Within said aspect, it also envisaged in the context of the present invention also provides a method to address a disease-associated target being significantly co-expressed on a pathophysiological and one or more physiological tissues by providing a multitargeting bispecific antigen-binding molecule of the format described herein, wherein the molecule addresses (i.) the target expressed both on the disease-associated and the physiological tissue and (ii.) a further target which is disease associated but not expressed on the physiological tissue under (i.), wherein the method preferably avoids the formation of intra-abdominal adhesions and/or fibrosis where such target is MSLN.

Within said aspect, it is also envisaged in the context of the present invention that the disease preferably is a tumorous disease, cancer, or an immunological disorder, comprising the step of administering to a subject in need thereof the antigen-binding molecule of the present invention, or produced according to the process of the present invention, wherein the disease preferably is acute myeloid leukemia, Non-Hodgkin lymphoma, Non-small-cell lung carcinoma, pancreatic cancer and/or Colorectal cancer.

Within said aspect, it is also envisaged in the context of the present invention that TAA1 and TAA2 are preferably selected from EpCAM and MSLN, MSLN and EpCAM, MSLN and CDH3, CDH3 and MSLN, FLT3 and CLL1, and CLL1 and FLT3.

In an eighth aspect, it is also envisaged in the context of the present invention to provide a kit comprising an antigen-binding molecule of the present invention, or produced according to the process of the present invention, a polynucleotide of the present invention, a vector of the present invention, and/or a host cell of the present invention.

In a ninth aspect, it is also envisaged in the context of the present invention to provide a molecule comprising at least one polypeptide chain, wherein the molecule comprises, from N-terminus to C-terminus:
(i.) a first binding domain which specifically binds to a first target cell surface antigen (e.g. TAA1),
(ii.) a second binding domain which specifically binds to a second target cell surface antigen (e.g. TAA2),
(iii.) a spacer entity,
(iv.) a third binding domain which specifically binds to an extracellular epitope of the human and/or the Macaca CD3ε chain, and
(v.) a fourth binding domain which specifically binds to an extracellular epitope of the human and/or the Macaca CD3ε chain,
wherein the spacer entity spaces apart the second and the third binding domains by more than about 50 Å.

### DESCRIPTION OF THE FIGURES

FIG. 1: Overview of multitargeting bispecific antigen-binding molecules disclosed in the invention. Domain arrangement in each molecule as follows: A: target binding domain×CD3 binding domain×spacer×target binding domain×CD3 binding domain; B: target binding domain×CD3 binding domain×spacer×CD3 binding domain×target binding domain; C: target binding domain×target binding domain×spacer×CD3 binding domain×CD3 binding domain; D: target binding domain×target binding domain×CD3 binding domain×CD3 binding domain×spacer; E: target binding domain×target binding domain×CD3 binding domain×spacer×CD3 binding domain; F: target binding domain×spacer×target binding domain×CD3 binding domain×CD3 binding domain

FIG. 2: FIG. 2 shows cytotoxicity curves and EC50 values of dual targeting CLL1-FLT3 bispecific antigen-binding molecules and mono targeting control bispecific antigen-binding molecules on double positive CHO huCLL1 huFLT3 target cells and single positive CHO huCLL1 or CHO huFLT3 target cells. Effector cells were unstimulated Pan T-cells. b.c.t: below calculation threshold

FIG. 3: FIG. 3A to H shows cytotoxicity curves of EpCAM MSLN T-cell engager molecules and mono targeting control T-cell engager molecules on double positive CHO huEpCAM huMSLN target cells and single positive CHO huEpCAM or CHO huMSLN target cells. Effector cells were unstimulated Pan T-cells.

FIG. 4: FIG. 4A shows Cytotoxicity curves of EpCAM MSLN T-cell engager molecules on double positive CHO huEpCAM huMSLN target cells and single positive CHO huEpCAM or CHO huMSLN target cells. Effector cells were unstimulated Pan T-cells. FIG. 4B shows Cytotoxicity curves of EpCAM MSLN T-cell engager molecules on double positive CHO huEpCAM huMSLN target cells and single positive CHO huEpCAM or CHO huMSLN target cells. Effector cells were unstimulated Pan T-cells. FIG. 4C shows Cytotoxicity curves of CLL1-FLT3 T-cell engager molecules on double positive CHO huCLL1 huFLT3 target cells and single positive CHO huCLL1 or CHO huFLT3 target cells. Effector cells were unstimulated Pan T-cells.

FIG. 5: FIG. 5 shows cytotoxicity curves of EpCAM MSLN T-cell engager molecules on double positive CHO huEpCAM huMSLN target cells and single positive CHO huEpCAM or CHO huMSLN target cells. Effector cells were unstimulated Pan T-cells.

FIG. 6: FIG. 6A shows cytotoxicity curves of CLL1-FLT3 T-cell engager molecules on double positive CHO huCLL1 huFLT3 target cells and single positive CHO huCLL1 or CHO huFLT3 target cells. Effector cells were unstimulated Pan T-cells. FIG. 6B shows cytotoxicity curves of EpCAM MSLN T-cell engager molecules on double positive CHO huEpCAM huMSLN target cells and single positive CHO huEpCAM or CHO huMSLN target cells. Effector cells were unstimulated Pan T-cells. FIG. 6C shows Cytotoxicity curves of CLL1-FLT3 T-cell engager molecules on double positive CHO huCLL1 huFLT3 target cells and single positive CHO huCLL1 or CHO huFLT3 target cells. Effector cells were unstimulated Pan T-cells.

FIG. 7: FIG. 7 shows cytotoxicity curves of CLL1-FLT3 (FIG. 7A) and CDH3-MSLN vs. CDH3 and MSLN monotargeting (FIG. 7G) T-cell engager molecules, respectively, on double positive CHO huCLL1 huFLT3 and GSU Luc CDH3 MSLN target cells after 48 h, and released cytokines IL-2, IL-6, IL-10, TNFα und IFNγ after 24 h (FIGS. 7B-F and H-L, respectively. Effector cells were unstimulated PBMC.

FIG. 8: FIG. 8 shows cytotoxicity curves and EC50 values of MSLN-CDH3 T-cell engager molecule 1 on double positive cell line HCT 116 (WT) and CDH3 respectively MSLN Knockout (KO) cell lines. Effector cells were unstimulated Pan T-cells.

FIG. 9: FIG. 9 shows cytotoxicity curves and EC50 values of MSLN-CDH3 T-cell engager molecule 1 on double positive cell line SW48 (WT) and CDH3 respectively MSLN Knockout (KO) cell lines. Effector cells were unstimulated Pan T-cells.

FIG. 10: FIG. 10 shows cytotoxicity curves of CLL1-FLT3 T-cell engager molecules on double positive CHO huCLL1 huFLT3 target cells and single positive CHO huCLL1 or CHO huFLT3 target cells. Effector cells were unstimulated Pan T-cells.

FIG. 11: FIG. 11 sows cytotoxicity curves of MSLN-CDH3 T-cell engager molecules and Mono-targeting T-cell engager molecules on naïve double positive GSU cells versus target-knockout GSU cells. Effector cells were unstimulated Pan T-cells.

FIG. 12: FIG. 12 shows cytotoxicity curves and EC50 values of CLL1-FLT3 T-cell engager molecules on double positive CHO huCLL1 huFLT3 target cells and single positive CHO huCLL1 or CHO huFLT3 target cells. Effector cells were unstimulated Pan T-cells.

FIG. 13: FIG. 13 shows cytotoxicity curves of EpCAM-MSLN T-cell engager molecules on double positive Ovcar8 Wildtype cells and single positive Ovcar8 MSLN KO or Ovcar8 EpCAM KO target cells. Effector cells were unstimulated Pan T-cells.

FIG. 14: FIG. 14 shows MSLN-CDH3 T-cell engaging cytotoxicity assays Effector cells: human unstimulated T cells with Target cells being (A molecule 1, B molecule 2) GSU wt, GSU KO CDH3, GSU KO MSLN and (C molecule 1, D molecule 2) HCT 116 wt, HCT 116 KO CDH3, HCT 116 KO MSLN.

FIG. 15: FIG. 15 shows an Overlay (x and y-normalized) of UV280 trace from Cation exchange chromatography of MSLN-CDH3 T-cell engager molecule 1 and 2.

FIG. 16: FIG. 16 shows in vivo dose-dependent tumor growth inhibition by CDH3×MSLN multitargeting bispecific antigen-binding molecule having SEQ ID NO 251 in a xenograft mouse model.

FIG. 17: FIG. 17(A-L) shows modeled mean and maximum distances over time (200 or 400 ns) between centers of mass of the two bispecific entities of an exemplary bispecific antigen-binding molecule with spacers G4S, scFc, scFc-scFc, (G4S)10, (EAAAK)10, has, PD1, ubiquitin, SAND, Beta-2-microblobulin, and HSP70-1. FIG. 17M shows visualizations of modelings of Beta-2-microblobulin, and HSP70-1. FIGS. 17N and O shows modeled mean and maximum distances over time (200 ns) between centers of mass of the two bispecific entities an exemplary bispecific antigen-binding molecules with scFc as spacer and with target binders MSLN-FOLR1 and MSLN-CDH19, respectively.

FIG. 18: FIG. 18 shows increased activity of a CD20×CD22 multitargeting antigen-binding molecule with two high affinity CD binders in the format according to the present invention.

FIG. 19: FIG. 19 shows an example cytotoxicity assay in which human T cells were incubated with the human gastric cancer cell line GSU Luc at an ET ratio of 10:1 for 72 hours. The resulting EC₅₀ values were within a similar range (2.078 μM for MSLN monotargeting molecule 1 (SEQ ID NO: 1183) versus 1.060 μM for CDH3-MSLN multitargeting molecule 2 (SEQ ID NO: 251), respectively, see Figure A).

FIG. 20: FIG. 20 shows histopathological glass slides which were scanned to generate whole slide images (WSI) in .svs format. WSI were viewed using Aperio eSlide Manager (Leica Biosystems, Version 12.3.3.5049, ª 2006-2017). Individual still images were grabbed using Snipping Tool (Microsoft Office) from the Aperio viewer and saved in jpg format. FIGS. 20A and B show liver of an cynomolgus monkey treated with 1.5 μg/kg monotargeting MSLN bispecific antigen-binding molecule (SEQ ID NO: 1183, molecule 1), magnification 4.4× (A) or with 1000 μg/kg multitargeting CDH3-MSLN bispecific antigen-binding molecule (SEQ ID NO: 251, molecule 2), magnification 8.4× (B). (B, C): Lung of an animal treated with 1.5 μg/kg molecule 1, magnification 4.4× (C) or with 1000 μg/kg molecule 2, magnification 8.4× (D).

FIG. 21: Cytotoxicity curves of single-chain vs. dual-chain MSLN-CDH3 T-cell engager molecules and corresponding Mono-targeting T-cell engager molecules, respectively, on naïve double positive GSU cells versus target-knockout GSU cells. Effector cells were unstimulated Pan T-cells.

FIG. 22: Cytotoxicity curves of MSLN-CDH3 T-cell engager molecules and Mono-targeting T-cell engager molecules on naïve double positive GSU cells versus target-knockout GSU cells, wherein the CD3 binders were varied, i.e. I2C, 12M2 and I2M instead of I2L. Effector cells were unstimulated Pan T-cells.

FIG. 23: FIG. 23(A-H) shows MSLN-CDH3 T-cell engaging cytotoxicity assays with Effector cells: human stimulated T cells and Target cells: HCT 116 wt, HCT 116 KO CDH3, HCT 116 KO MSLN, wherein selectivity gaps of CDH3 epitope cluster D4B is compared with CDH3 epitope clusters D1B, D2C and D3A.

FIG. 24: FIG. 24(A-E) shows MSLN-CDH3 T-cell engaging cytotoxicity assays with Effector cells: human stimulated T cells and Target cells: CHO hu CDH3 (+) & MSLN (+), CHO hu CDH3 (+), CHO hu MSLN (+), wherein selectivity gaps of MSLN epitope cluster E1 is compared with MSLN epitope cluster E2/E3.

FIG. 25: human CDH3, sequence below: mouse CDH3 with transmembrane and cytoplasmic domain of EpCAM. Sequence alignment of the CDH3 protein shows each human sequence part (D1, D2, D3, D4, D5 and the respective subparts A, B and C) that was replaced with the corresponding mouse sequence and which amino acids differ between the two species.

FIG. 26: Flow Cytometry Binding Analysis of CDH3 Antibody and T Cell Engager K3T on Transfected CHO Cells Expressing Full-length Human CDH3 or Mouse CDH3×EpC Protein or Human/Mouse Chimeric CDH3×EpC Protein Constructs

FIG. 27: Sequence alignment of the MSLN protein shows each human sequence epitope section (E1, E2, E3, E4, E5 and E6) that was replaced with the corresponding mouse sequence and which amino acids differ between the two species.

FIG. 28: Flow Cytometry Binding Analysis of T Cell Engager K3T and F5Q on Transfected CHO Cells Expressing Full-length Human MSLN Protein or Full-length Mouse MSLN Protein or Human/Mouse Chimeric MSLN Protein Constructs

### DETAILED DESCRIPTION

In the context of the present invention, a multitargeting bispecific molecule is provided comprising at least five distinctive structural entities, i.e. (i.) a first domain binding to a target cell surface antigen (e.g. a first tumor associated antigen, TAA), (ii.) a second domain binding to an extracellular epitope of the human—and preferably non-human, e.g. Macaca—CD3ε chain, wherein the first binding domain and the second binding domain together form a first bispecific entity, (iii.) a spacer which connects but spaces apart the first bispecific entity from a second bispecific entity comprising (iv.) a third domain binding to the same or preferably a different target cell surface antigen (e.g. a second TAA), and (v.) a fourth domain binding to an extracellular epitope of the human—and preferably non-human, e.g. Macaca—CD3ε chain. Molecules of the format of the present invention typically exhibit the advantage to be characterized by avidity-driven potency and specificity from two targets being co-expressed on the target cell, which typically leads to a reduction of undesired cytokine release (and associated clinically relevant side effects such as CRS) while at the same time ensuring effective antitumor activity, preferably also in solid tumors such as colorectal cancer, non-small-cell lung carcinoma and pancreatic cancer.

It is a surprising finding in the context of the present invention that bispecific (T-cell engaging) multitargeting molecules according to the present invention provides a double avidity effect, both on the target cell binder and the effector cell binder side due to their specific format which leads to an efficient each other complementing target cell kill. This effect is facilitated by the molecule format specifically targeting two (different) antigens on one target cell, such as a cancer cell, and in contrast, by significantly less targeting non-target cells while mediating a potent T-cell response against said target cell at the same time. By being capable to address two target antigens at the same time, the likeliness of targeting a target cell associated with a disease instead of a physiologic cell is greatly increased when two TAAs are chosen which are typically associated with a target cell associated with a disease. Hence, a T-cell engaging multitargeting molecule according to the present invention, which is typically singe-chained, both provides improved efficacy and safety with regard to existing bispecific antibodies or antibody-derived constructs which are T-cell engaging. Said advantageous properties are preferably achieved by the fact that the multitargeting bispecific molecules of the present invention comprise two bispecific entities comprising each a target binding domain and an effector (CD3) binding domains which can act in a pathophysiologic environment without (e.g. sterically) hindering each other while complementing each other at the same time. Said action of the two bispecific entities within the one multitargeting bispecific molecule of the present invention from each other means that the target binding domain (e.g. the first domain) and the effector CD3 binding domain (e.g. the second domain) of the first bispecific entity can interact with their respective binding partners to form a cytolytic synapse between target cell and T-cell, without disturbing interaction of or with the target binding domain (e.g. the third domain) and the effector domain (e.g. the forth domain) of the second bispecific entity. However, in order to provide the desired action and, in consequence, therapeutic function, preferably both target binding domains of both the first and the second bispecific entity must engage their respective target in order to involve the effector CD3 binding domains of the first and second bispecific entity completely. Further, it was a surprising finding that the two respective bispecific entities must be functionally preserved by structural separation in the molecule format in a specific manner in order to benefit from the double avidity effect required to achieve the extraordinary efficacy described and safety implied herein.

As a secondary effect in addition or alternatively to the herein described increased specificity, and therefore safety, the likeliness of targeting a target cell such as a cancer cell by a multitargeting antigen-binding molecule versus a monotargeting molecule is greatly increased once such target cell has undergone antigen loss and, thus, is prone to tumor escape from effective anti-tumor therapy because one valid antigen to target remains on the cell which has undergone antigen escape. Said effect in terms of increased activity compared to molecules comprising only one CD3 binder and/or target binder and do not comprise the two linked but spaced apart bispecific entities is preferably achieve when both CD3 binders are of high affinity, such as a CD3 binding domain comprising a VH and VL of, for example, SEQ ID NOs 67 and 68, respectively, linked by a linker of SEQ ID NO 1 or 3.

The above-specified finding underlying the present invention is surprising in view of the teaching of the prior art. For example, antigen-binding formats comprising more than one target binding domain and effector binding domain, respectively, are known in the art, e.g. the Adaptir™ format. However, such formats do not provide two bispecific entities which can individually interact with their respective target and effector and work together at the same time and, consequently, cannot achieve the effect of double avidity on both the target binder and the effector binder side to the extent of effectively provide a large selectivity gap to the advantage of the multitargeting molecule. According to the present invention, the two bispecific entities must be spaced apart from each other by a certain distance, preferably of at least 50 Å, more preferably at least 60, 70, 80, 90 or at least 100 Å. The indicated distance [Å] between the two bispecific entities is typically understood in the context of the present invention as the distance between the centers of mass of the two bispecific entities, respectively. In general, the center of mass (COM) of a distribution of mass (here, a bispecific entity comprising a binding domain which binds to a target cell surface antigen and a binding domain which binds to an extracellular epitope of the human—and preferably the Macaca—CD3ε chain, both binding domains preferably in scFv or, alternatively, in scFab format and linked by a peptide linker) in space is understood as the unique point where the weighted relative position of the distributed mass sums to zero. The distance is typically determined by molecular modeling making use of generally accepted modeling programs (MD/visualization software) which can identify COMs given input structures and such as PyMOL (The PyMOL Molecular Graphics System, Version 2.3.3. Schrödinger, LLC.) which is typically based on ensembles of snapshot structures from MD simulations. The mass of each atom is typically part of an underlying “force field” as generally known in the art. Alternatively and/or additionally, distances can be determined by crystallography, cryo electron microscopy, or nuclear magnetic resonance analytic technology.

A typical approach of obtaining distances through molecular modeling as given in the present invention is as follows:
1) Obtaining an atomistic structure of the complete bispecific antigen-binding molecule. Structure sources may be selected from the group consisting of:
   a. Protein X-ray crystallography with resolution preferably below 5 Å enabling visibility of amino acid backbones and side-chains;
   b. Cryogenic electron microscopy (cryo-EM) with resolution preferably below 5 Å enabling visibility of amino acid backbones and side-chains;
   c. In silico homology modeling of the entire molecule based on a single, highly-homologous crystal and/or cryo-EM structure (preferably above 60% sequence identity);
   d. In silico homology modeling involving linking 2 or more experimental structures. The structures are preferably identical or highly homologous (preferably above 60% sequence identity) to domains found in the complete bispecific antigen-binding molecule. In case of lack of experimental linker conformations, the model is preferably refined in an explicit-solvent Molecular Dynamics (MD) simulation (simulation length of preferably at least 100 ns unless energy convergence is obtained faster). The simulation is carried out with a state-of-the-art software (e.g. Schrodinger, Amber, Gromacs, NAMD or equivalent) with parameters corresponding to room temperature and pressure. No artificial forces are applied during the simulation (i.e. preferably excludes methods such as metadynamics or steered molecular dynamics). Similarly, preferably no artificial geometrical restraints are imposed on the molecule.
2) Identifying centers of mass (COM) of the relevant molecule domains. This is typically performed with the used MD software or with visualization tools such as PyMOL or VMD. The centers of mass can be defined as a pseudo-atoms or non-hydrogen atoms closest to the true COM. Inter-domain linkers are typically not considered as part of the domain.
3) Using the same software, report the distance (in Angstrom, Å) between the two COMs. If an MD simulation was used to refine a homology model (as described in 1d), the median distance over multiple simulation snapshots is reported. To further diminish potential inaccuracy of the initial model, at least the first 10% of the simulation, preferably up to 50% if the signal significantly changes, are omitted when calculating the median distance between COMs and when extracting the snapshots for visualizing the MD simulation.

If not indicated otherwise, distances [A] in the context of the present invention are median distances as determined by MD simulations.

The preferred distance between the first and the second bispecific entity as disclosed herein is facilitated by a spacer entity (in short spacer) between the two bispecific entities which spaces the two bispecific entities apart and keeps them in a separated position. The spacer is of a certain size, preferably at least more than 5 kDa, more preferably at least about 10, 15, 20, 25, 30, 35, 40, 45 or even at least 50 kDa and hereby prevents an undesired interaction of the two separated bispecific entities. The preferred range in molecular size of the spacer is about 15 to 200 kDa, preferably about 15 to 150 kDa, in order to facilitate the separation of the two bispecific entities according to the present invention and to maintain a high overall activity of the molecule. Typically, too large spacers, e.g. larger than about 200 kDa, may impact the ability of the two bispecific entities to bind to two target surface structures on the same target cell which in turn may reduce the overall activity of the molecule against the target cell. Hence, the typical maximum preferred size in terms of molecular weight of the spacer is about 200 kDa, preferably about 150 or 120 kDa and even more preferably about 100 kDa. A typical spacer of maximum preferred size is a double scFc domain as disclosed herein (two scFc linked to each other forming one larger single chain spacer) of about 105.7 kDa. Example sizes of spacers which typically sufficiently separate the two bispecific entities are PSI domain of Met-receptor of about 5.3 kDa, ubiquitin of about 8.6 kDa, fibronectin type III domain from tenascin of about 10.1 kDa, SAND domain of about 11 kDa, beta-2-microglobulin of about 11.9 kDa, Tim-3 (aa 24-130) of about 12.2 kDa, MiniSOG of about 13.3 kDa, SpyCatcher of about 12.1 kDa associated with SpyTag of about 1.7 kDa linked together preferably via isopeptide bond formation to form a two-chain-spacer of about 13.8 kDa, VHH antibody lama domain of about 14 kDa, PD-1 binding domain from human programmed cell death 1 ligand (PDL1) of about 14.4 kDa, granulocyte-macrophage colony stimulating factor (GM-CSF) of about 14.5 kDa, intrleukin-4 of about 15 kDa, interleukin-2 of about 15.4 kDa, CD137L (4-1BBL; TNFSF9) cctodomain of about 17.7 kDa, programmed cell death protein 1 (PD-1) of about 16.6 kDa, green fluorescent protein (GFP) of about 26.3 kDa, single chain Fc region (scFc) as described herein of about 52.8 kDa (about 54.6 kDa with N- and C-terminal linkers (G₄S)₃, respectively), human serum albumin (HSA) of about 66.5 kDa (about 68.3 kDa with N- and C-terminal linkers (G₄S)₃, respectively) and double scFc (two scFc linked to cach other forming one larger single chain spacer) of about 105.7 kDa (about 107.5 kDa with N- and C-terminal linkers (G₄S)₃, respectively). In general, the more rigid the spacer is, the less is the median distance required which otherwise has to include a safety margin for flexible spacers.

Also, a preferred spacer in the context of the present invention, such as a globular domain, typically has a N- and a C-terminus which are spatially not too close to each other in order to efficiently space apart the two bispecific entities according to the invention. In this regard, spacers typically show a distance between the N- and the C-terminus which is significantly larger than 10 Å. A distance between N- and C-terminus of the spacer which is lower or about 10 Å is considered “close”. Hence, a spacer in the context of the present invention preferably has a distance between the alpha-carbon atoms of the first amino acid located at the N-terminus and the last amino acid at the C-terminus of at least 20 Å, more preferably at least 30 Å, even more preferably at least 50 Å, which distance typically ensures to space the first and the second bispecific entity apart by at least 50 Å as described herein. Alpha-carbon (α-carbon) is understood herein as a term that applies to proteins and amino acids. It is the backbone carbon before the carbonyl carbon atom in the molecule. Therefore, reading along the backbone of a typical protein would give a sequence of —[N—Ca-carbonyl C]n-etc. (when reading in the N to C direction). The α-carbon is where the different substituents attach to each different amino acid. That is, the groups hanging off the chain at the α-carbon are what give amino acids their diversity. Hence, in the context of the present invention, a spacer is less preferred, even if it has a size of at least 5 kDa and a length of more than 50 aa if the distance between the alpha-carbon atoms of the first amino acid located at the N-terminus and the last amino acid at the C-terminus is too close, i.e. if it is only, e.g., about 10 Å. For example, preferred spacers show typical distances between the alpha-carbon atoms of the first amino acid located at the N-terminus and the last amino acid at the C-terminus as follows: scFc (based on 5G4S crystal structure) 89 Å, HSA (based on 5VNW crystal structure): 77 Å, ubiquitin (based on 1UBQ crystal structure): 37 Å and SAND (based on 10QJ crystal structure): 32 Å. In contrast, HSP70-1 (based on 3JXU crystal structure) shows only a distance of 9 Å between the alpha-carbon atoms of the first amino acid located at the N-terminus and the last amino acid at the C-terminus. At the same time, HSP70-1 provides only a median distance between the COMs of first and the second bispecific entity in the context of the present invention of about 48 Å which is below the threshold of 50 Å median distance, and significantly below the typically about 60-100 Å median distance between the COMs of the two bispecific entities as facilitates by preferred spacers such as scFc. HSA, ubiquitin and SAND. Thereof, scFc (SEQ ID NO: 25) is preferred.

Alternatively, a non-globular but rigid linker may serve as a spacer in the context of the present invention which spaces apart the two bispecific entities. Such linkers comprise (PA)25P (SEQ ID NO: 1097) and A(EAAAK)4ALEA(EAAAK)4A (SEQ ID NO: 1096), even if the Mw is below 5 kDa (here 4.3 kDa) and the amino acid length is only about or below 50 (51 and 46 aa, respectively). However, such spacers are typically less preferred than globular domains which preferably additionally increase half-life.

As it is also contemplated within the context of the present invention, the spacer between the two bispecific entities is a polypeptide which typically comprises more than 50 amino acids, preferably at least about 75, 100, 150, 200, 250, 300, 350, 400, 450 or at least 500 amino acids. The preferred range in amino acid length of the spacer is about 100 to 1500 amino acids, preferably about 100 to 1000 amino acids, more preferably about 250 to 650 amino acids in order to facilitate the separation of the two bispecific entities according to the present invention. This is to preferably maintain a high overall activity of the entire molecule according to the present invention (not necessarily of the individual and spaced-apart bispecific entities, which may have low affinities (and low activities) individually in order to increase specificity for double positive target cells) which is typically be below 20 μM, preferably below 5 μM, more preferably below 1 μM. Typically, too large spacers, e.g. longer than about 1500 amino acids, may impact the ability of the two bispecific entities to bind to two target surface structures on the same target cell which in turn may reduce the overall activity of the molecule against the target cell. Hence, the typical maximum preferred length of the spacer is about 1500 amino acids, more preferably about 1000 amino acids. Example amino acid lengths of spacers which sufficiently separate the two bispecific entities are PD-1 of about (ECD 25-167) 143 aa, scFc as described herein of about 484 aa (about 514 aa with N- and C-terminal linkers (G₄S)₃, respectively), HSA of about 585 aa (about 615 aa with N- and C-terminal linkers (G₄S)₃, respectively), and double scFc of about 968 aa (about 998 aa with N- and C-terminal linkers (G₄S)₃, respectively). Further spacers include, ubiquitin of about 76 aa, fibronectin type III domain from tenascin of about 90 aa, SAND domain of about 90 or 97 aa, beta-2-microglobulin of about 100 aa, Tim-3 (aa 24-130) of about 108 aa, MiniSOG of about 115 aa, SpyCatcher of about 113 aa associated with SpyTag of about 14 aa linked together preferably via isopeptide bond formation to form a two-chain-spacer of about 127, VHH antibody lama domain of about 129 aa, PD-1 binding domain from human programmed cell death 1 ligand (PDL1) of about 126 aa, granulocyte-macrophage colony stimulating factor (GM-CSF) of about 127 aa, interleukin-4 of about 129 aa, interleukin-2 of about 133 aa, CD137L (4-1BBL; TNFSF9) ectodomain of about 167 aa, and green fluorescent protein (GFP) of about 238 aa.

The composition and arrangement of the preferred spacer amino acid sequences preferably confer a certain rigidity and are not characterized by high flexibility. Rigidity in the context of the present invention is typically present when a spacer of more than 50 aa and/or a molecular weight over 5 kDa facilitates a maximum distance between the centers of mass of the two bispecific entities in a molecule according to the present invention which is smaller than 200% (or 2-fold) the median distance. Accordingly, a preferred rigid spacer in the context of the present invention does not extend further than about 100% of its median length, more preferably not more than about 80% (each calculated as distance between centers of mass of the two bispecific entities). Hence, a preferred rigid spacer in the context of the present invention which spaces apart the two bispecific entities by about 100 Å (median distance) does not extend further than to 200 Å (maximum distance). For example, a typical median distance between centers of mass of the bispecific entities of a molecule having the format of the present invention comprising a scFc (such as SEQ ID NO: 25) as spacer is about 101 Å. However, a maximum distance in such a case is typically about 182 Å, i.e. not more than about 100% or even only about 80% with respect to the median distance. Such a spacer is considered rigid in the context of the present invention. In contrast, a molecule comprising a (G₄S)₁₀ (SEQ ID NO: 8) as spacer, which is a liner polypeptide without a e.g. globular structure, shows a typical a median distance of about 48 Å and a maximum distance of about 179 Å. Hence, such a spacer as (G₄S)₁₀ shows a high flexibility and not the rigidity of a preferred spacer as advantageous feature according to the present invention. In this regard, spacer amino acid sequences may typically be rich in proline and less rich in serine and glycine. Especially envisaged are spacers which are folded polypeptides e.g. of secondary order (e.g. helical structures) or of ternary order forming e.g. three dimensional protein domains structures which in turn ensure a certain rigidity by their constitution and preferably confer further advantageous effects such as in vivo half-life extension of the multitargeting bispecific molecule as a therapeutic agent. Typical domain structures comprise hydrophobic cores with hydrophilic surfaces. In the context of the present invention, proteins having a structure of a globular protein are preferred as spacers. Globular proteins are understood in the context of the present invention to be spherical (“globe-like”) proteins and are one of the common protein types. Globular proteins in the context of the present invention may be characterized by a globin fold. Spacers comprising an Fc domain or parts or a multiple thereof, a PD-1 or an HSA domain are in particular envisaged. Also envisaged are spacers which comprise combinations of different globular proteins or parts thereof, which even more preferably comprise a Fc receptor binding function in order to increase the half-life of the molecule according to the present invention. The format described herein with the separation of the two bispecific entities has distinctive advantages. If only one target is present which is addressed by the first binding domain, then the first domain “uses” only the second domain engage a T cell but not the fourth domain, or alternatively, the third domain uses the fourth but not the second (or to a much lesser extend due to the spacer). If only one target is present, the Kd of preferably low affinity CD binder as disclosed herein prevents efficient T-cell engagement. Thus, selectivity is increased with respect to other (dual) targeting molecules.

If both targets are present, the BiTE² binds more firmly to the target cell (by avidity gain) and both I2L can be used to engage T cells (also by avidity gain), for example the second domain binding to a CD3 domain on an effector T cell and the third domain binding to a target antigen are less likely to form a cytolytic synapse and therefore do not act together as a bispecific entity which would otherwise lead to less beneficial cytotoxic activity profile. This has the advantage that the first and the fourth domain are not left “useless” which would mean that the full effect of the double avidity by double binding of a target and an effector binding domain, respectively, could not be made use of. Likewise, the first domain binding to a target antigen and the fourth domain binding to a CD3 domain on an effector T cell are prevented from theoretical interaction which would eventually render the second and the third domain useless for forming a cytolytic synapse with their intended “partner domains” in their respective bispecific entities.

Typically, the advantageous avidity effect conferred by a multitargeting bispecific molecule according to the present invention is indicated by a differential activity factor or “selectivity gap” between the activity of the molecule on double positive cells, i.e. a target cell which carries (i.) two different targets which combination is overexpressed on the cell type to be targeted and being associated with a particular disease and/or (ii.) one target at overexpressed levels. In either case, a molecule according to the present invention targeting two (preferably different) targets at the same time, will preferably bind to such a target cell in comparison to a non-target cell expressing either only one of two targets or the one target at lower expression levels and, in consequence, will induce a more pronounced T cell response. As it preferred for a multitargeting bispecific molecule of the present invention, the activity in terms of increased cytotoxicity as determined, for example, by lower EC50 values, is at least 100 times larger on target cells (e.g. characterized by expressing both different targets or the one target at high levels) than on non-target cells (e.g. characterized by expressing only one of two targets or the one target only at low levels). Said selectivity gap in the context of the present invention is preferably larger than 100 times. It is envisaged in the context of the present invention that the selectivity gap (which can also be defined as activity gap) is at least 250, 500, 750 or even 1000 times which greatly improves efficacy and safety of the present multitargeting bispecific molecule in comparison to monotargeting bispecific molecules of various formats.

A further aspect envisaged in the context of the present invention is the further support of the double avidity effect conferred by the format of the multitargeting antigen-binding molecule by means of a low affinity, preferably both of the target antigen binders and the CD3 effector binders. In the context of the present invention, a CD3 binder with an affinity below KD 1.2×10⁸ is preferred. Especially preferred are CD3 binders which have an activity which is 10 times lower, more preferably 50 times lower or even more preferred 100 times lower than that of a CD3 binder having a KD 1.2×10-8. Without wanting to be bound be theory, the avidity effect is contemplated to be more pronounced when two binders with relatively balanced, i.e. typically two low affinity binders bind to two targets on the same target cell compared to binders with mixed or, typically, higher affinity which would trigger cytolytic activity also if only one target on a cell was bound which could, for example, be a physiologic non-target cell which should not be targeted in order to avoid off-target toxicity and related side effects.

Accordingly, the multitargeting bispecific antigen-binding molecules according to the present invention which bind to two (preferably different) targets on a target cell in order to show significant cytotoxic activity preferably do show less side effects than monotargeting bispecific antigen-binding molecules which bring together effector T cell and target cell. This is demonstrated, for example, by a significant reduction in release of key cytokines IL-2, IL-6, IL-10, TNFa and IFNg which are an indicator for side effects on a clinical stage. For example, release of IL-6 is typically reduced upon use of a multitargeting bispecific antigen-binding molecule according to the present invention with respect to a corresponding monotargeting bispecific molecule. As it is known in the art, interleukin 6 (IL-6) seems to hold a key role in CRS pathophysiology since highly elevated IL-6 levels are seen in patients with CRS (Shimabukuro-Vornhagen et al. Journal for ImmunoTherapy of Cancer (2018) 6:56). As CRS is a serious side effect in immunotherapies, such reduction is an indication for less CRS in the clinical stage.

Further, the multitargeting bispecific antigen-binding molecules according to the present invention which bind to two (preferably different) targets on a target cell in order to show significant cytotoxic activity preferably do show less side effects than monotargeting bispecific antigen-binding molecules in terms of toxicity tissue damage. It has been a surprising finding that a multispecific molecule of the format as described herein shows higher tolerability, i.e. higher doses can be administered than corresponding monotargeting bispecific molecules without clinical finings such as tissue damage examined by histopathological examination. For example, a dose of 1.5 μg/kg of a MSLN monotargeting bispecific antigen-binding molecule (SEQ ID NO: 1183) was not tolerated and resulted in mortality whereas a dose of 0.1 μg/kg was tolerated. Conversely, a multitargeting CDH3-MSLN bispecific molecule (SEQ ID NO: 251) according to the present invention was tolerated at doses of up to 1000 μg/kg. Histopathological changes seen with the monotargeting molecule were generally more severe at doses of 1.5 μg/kg than those with the multitargeting molecule at 1000 μg/kg, respectively. Adhesions or irreversible fibrotic changes as induced by the monotargeting molecule were absent after treatment with the multitargeting molecule. Therefore, the tolerability of a multitargeting molecule according to the present invention is, e.g., 600 (histopathology) to, e.g., 10.000 (tolerated dose) times higher than for a corresponding monotargeting molecule despite equivalent in vitro potency against tumor cells. Hence, the multitargeting molecules of the present invention are particularly suitable in therapeutic settings, where targets are addressed which are significantly present not only on disease-associated (pathophysiological) but also or even predominately on physiological tissues which should, however, not be targeted by a cytotoxic immunotherapy. This is the case, e.g., for MSLN which is typically expressed in mesothelial cells which form the lining of several body cavities: the pleura (pleural cavity around the lungs), peritoneum (abdominopelvic cavity including the mesentery, omenta, falciform ligament and the perimetrium) and pericardium (around the heart). Addressing targets like MSLN by cytotoxic immunotherapy bears the risk of severe side effects such as intra-abdominal adhesions and/or fibrosis. Intra-abdominal adhesions are understood herein as pathologic scars formed between intra-abdominal organs. Adhesions can occur in the presence of intraperitoneal inflammation and cause peritoneal surfaces to adhere to each other. Adhesions can cause problems if the scarring limits the free movement of organs (Mutsaers S. E., Prele C. M. Pengelly, S., Herrick, S. E. Mesothelial cells and peritoneal homeostasis. Fertil Steril 2016, 106(5) 1018-1024). Fibrosis is understood herein as a common pathological outcome of several etiological conditions resulting in chronic tissue injury and is usually defined as an excessive deposition of extracellular matrix (ECM) components, leading with time to scar tissue formation and eventually organ dysfunction and failure (Maurizio Parola, Massimo Pinzani, Pathophysiology of Organ and Tissue Fibrosis, Molecular Aspects of Medicine 2019. (65) 1). Hence, the present invention also provides a method to address a disease-associated target being significantly co-expressed on a pathophysiological and one or more physiological tissues by providing a multitargeting bispecific antigen-binding molecule of the format described herein, wherein the molecule addresses (i.) the target expressed both on the disease-associated and the physiological tissue and (ii.) a further target which is disease associated but not expressed on the physiological tissue under (i.), wherein the method preferably avoids the formation of intra-abdominal adhesions and/or fibrosis where such target is MSLN.

It is envisaged that the bispecific antigen-binding molecules according to the present invention have cross-reactivity to, for example, cynomolgus monkey tumor-associated antigens such as CDH3, MSLN, CD20, CD22, FLT3, CLL1, and EpCAM. It is in particular envisaged in the context of the present invention that two targets can be addressed by one multitargeting bispecific antigen-binding molecule simultaneously.

Alternatively and besides the major advantage of increasing selectivity as described herein, dual targeting can mitigate lack of accessibility of one target when targeting the remaining target can trigger a sufficient residual effect. Examples are (i) the presence of soluble target which would “mask” the target on the target cell by binding the antigen-binding molecule without allowing the remaining molecule any therapeutic effect and (ii) antigen loss (lowering target expression on target cell) as the driving factor for tumor escape.

For example, a multitargeting antigen-binding molecule according to the present invention such as a construct directed against MSLN as TAA1 and CDH3 as TAA2 is suitable for use in the treatment, amelioration or prevention of cancer, in particular cancer selected from the group consisting of, lung carcinoma, head and neck carcinoma, a primary or secondary CNS tumor, a primary or secondary brain tumor, primary CNS lymphoma, spinal axis tumors, brain stem glioma, pituitary adenoma, adrenocortical cancer, esophagus carcinoma, colon cancer, breast cancer, ovarian cancer, NSCLC (non-small cell lung cancer), SCLC (small cell lung cancer), endometrial cancer, cervical cancer, uterine cancer, transitional cell carcinoma, bone cancer, pancreatic cancer, skin cancer, cutaneous or intraocular melanoma, hepatic cancer, biliary duct cancer, gall bladder cancer, kidney cancer, rectal cancer, cancer of the anal region, stomach cancer, gastrointestinal (gastric, colorectal, and duodenal) cancer, cancer of the small intestine, biliary tract cancer, cancer of the urethra, renal cell carcinoma, carcinoma of the endometrium, thyroid cancer, testicular cancer, cutaneous squamous cell cancer, melanoma, stomach cancer, prostate cancer, bladder cancer, osteosarcoma, mesothelioma, Hodgkin's Disease, non Hodgkins's lymphoma, chronic or acute leukemia, chronic myeloid leukemia, lymphocytic lymphomas, multiple myeloma, fibrosarcoma, neuroblastoma, retinoblastoma, and soft tissue sarcoma.

It is especially envisaged in the context of the present invention that a multitargeting antigen-binding molecule which preferably addresses two different target cell surface antigens thereby is very specific for its target cell and, therefore, preferably safe in its therapeutic use. Efficacy in terms of tumor growth inhibition has been demonstrated in vivo in a mouse model.

Preferred target cell surface antigens in the context of the present invention are, MSLN, CDH3, FLT3, CLL1, EpCAM, CD20, and CD22. Typically, target cell surface antigens in the context of the present invention are tumor associated antigens (TAA). B-lymphocyte antigen CD20 or CD20 is expressed on the surface of all B-cells beginning at the pro-B phase (CD45R+, CD117+) and progressively increasing in concentration until maturity. CD22, or cluster of differentiation-22, is a molecule belonging to the SIGLEC family of lectins. It is found on the surface of mature B cells and to a lesser extent on some immature B cells. Fms like tyrosine kinase 3 (FLT3) is also known as Cluster of differentiation antigen 135 (CD135), receptor-type tyrosine-protein kinase FLT3, or fetal liver kinase-2 (Flk2). FLT3 is a cytokine receptor which belongs to the receptor tyrosine kinase class III. CD135 is the receptor for the cytokine Flt3 ligand (FLT3L). The FLT3 gene is frequently mutated in acute myeloid leukemia (AML). C-type lectin-like receptor (CLL1), also known as CLEC12A, or as MICL. It contains an ITIM motif in cytoplasmic tail that can associate with signaling phosphatases SHP-1 and SHP-2. Human MICL is expressed as a monomer primarily on myeloid cells, including granulocytes, monocytes, macrophages and dendritic cells and is associated with AML. Mesothelin (MSLN) is a 40 kDa protein that is expressed in mesothelial cells and overexpressed in several human tumors. Cadherin-3 (CDH3), also known as P-Cadherin, is a calcium-dependent cell-cell adhesion glycoprotein composed of five extracellular cadherin repeats, a transmembrane region and a highly conserved cytoplasmic tail. It is associated with some types of tumors. Epithelial cell adhesion molecule (EpCAM) is a transmembrane glycoprotein mediating Ca2+-independent homotypic cell-cell adhesion in epithelia. EpCAM has oncogenic potential and appears to play a role in tumorigenesis and metastasis of carcinomas.

Further, it is envisaged as optionally but advantageously in the context of the present invention that the multitargeting antigen-binding molecule is provided with a spacer, preferably a globular protein structure such as a scFc domain, which also increases the molecule's half-life and enables intravenous dosing that is administrated only once every week, once every two weeks, once every three weeks or even once every four weeks, or less frequently.

In order to determine the epitope(s) of preferred multitargeting antigen-binding molecules according to the present invention directed, e.g. to the CDH3, MSLN or CD20 epitope, mapping was conducted as described herein. Preferred bispecific antigen-binding molecules having a target binder for CD20 are directed to all the of the epitope cluster E1A, E2B and E2C. An epitope cluster is understood herein as a stretch of amino acids (as disclosed herein and defined by their position according to the Kabat) within a target (as disclosed herein and defined by their position according to the Kabat) to which target a the whole the a target binder of a multitargeting bispecific antigen-binding molecule as described herein does essentially no longer bind, if said stretch of amino acid of the human target is replaced by a corresponding stretch of amino acids of the murine target. Therefore, said method of epitope clusters is understood herein as murine chimere sequence analysis. The method has been described, e.g. by Münz et al. Cancer Cell International 2010, 10:44 and was applied as described in detail in the examples with respect to CDH3 and MSLN.

The preferred epitope cluster is D4B for CDH3 as described herein and E1 for MSLN as described herein. As exemplified in the examples, selectivity gaps of multitargeting bispecific antigen-minding molecules of the present invention (with respect to comparable monotargeting bispecific antigen-binding molecules) are typically even larger and, hence, more preferably, if the MSLN target binder addresses the E1 epitope cluster and if the CDH3 target binder addresses the D4B epitope cluster. While addressing other epitope clusters also leads to surprisingly high selectivity gaps and the associated advantages in terms of efficacy and tolerability/safety, selectivity gaps are especially high and, thus preferred for molecules which comprise target binders which address E1 and D4B. Such molecules comprise, for example, a molecule with a MSLN target binder comprising CDR H1-H3 of SEQ ID NO 774 to 776 and CDR L1-L3 of 777 to 779 (and corresponding VH and VL of 780 and 781), CDR H1-H3 of SEQ ID NO 782 to 784 and CDR L1-L3 of 785 to 787 (and corresponding VH and VL of 788 and 789), CDR H1-H3 of SEQ ID NO 806 to 808 and CDR L1-L3 of 809 to 811 (and corresponding VH and VL of 812 and 813), CDR H1-H3 of SEQ ID NO 838 to 840 and CDR L1-L3 of 841 to 843 (and corresponding VH and VL of 844 and 845), CDR H1-H3 of SEQ ID NO 862 to 864 and CDR L1-L3 of 865 to 867 (and corresponding VH and VL of 868 and 869), CDR H1-H3 of SEQ ID NO 894 to 896 and CDR L1-L3 of 897 to 899 (and corresponding VH and VL of 900 and 901), CDR H1-H3 of SEQ ID NO 950 to 952 and CDR L1-L3 of 953 to 955 (and corresponding VH and VL of 956 and 957), CDR H1-H3 of SEQ ID NO 1030 to 1032 and CDR L1-L3 of 1033 to 1035 (and corresponding VH and VL of 1036 and 1037), or CDR H1-H3 of SEQ ID NO 86 to 88 and CDR L1-L3 of 89 to 91 (and corresponding VH of 92 and VL 93 or 94). A preferred example for a CDH3 binder binding to the preferred DB4 epitope cluster comprises CDR H1-H3 of SEQ ID NO 194, 432 and 196 and CDR L1-L3 of 197 to 199 (and corresponding VH and VL of 433 and 200). Further target binder which preferably bind to the preferred epitope cluster of D4B are, e.g., identified herein as CH3 15-E11 CC and CH3 24-D7 CC.

It is particular surprising that a multitargeting antigen-binding molecule according to the present invention is capable, to bind, preferably simultaneously to two different targets. Simultaneous binding has been demonstrated herein for several targets. However, this is surprising given the typically typical distance between the targets. For example, CD20 comprises two small extra cellular domains of only 6 aa and 47 aa. In contrast, CD22 comprises a 7 Ig domain long extracellular domain with 676 aa. However, despite the significantly different extracellular size and setup, a multitargeting antigen-binding molecule according to the present intention may successfully address both TAAs CD20 and CD22 at the same time for the benefit of increased efficacy and less toxicity.

An exemplary general arrangement of preferred “building blocks” of VH and VL of target and CD3 binder, respectively, as well as of preferred linkers and spacers as all disclosed herein, which together form the multitargeting bispecific antigen-binding molecule, can be summarized as follows:

It is envisaged in the context of the present invention, that preferred multitargeting antigen-binding molecules do not only show a favorable ratio of cytotoxicity to affinity, but additionally show sufficient stability characteristics in order to facilitate practical handling in formulating, storing and administrating said constructs. Sufficient stability is, for example, characterized by a high monomer content (i.e. non-aggregated and/or non-associated, native molecule) after standard preparation, such as at least 65% as determined by preparative size exclusion chromatography (SEC), more preferably at least 70% and even more preferably at least 75%. Also, the turbidity measured, e.g., at 340 nm as optical absorption at a concentration of 2.5 mg/ml should, preferably, be equal to or lower than 0.025, more preferably 0.020, e.g., in order to conclude to the essential absence of undesired aggregates. Advantageously, high monomer content is maintained after incubation in stress conditions such as freeze/thaw or incubation at 37 or 40° C. Even more, multitargeting antigen-binding molecules according to the present invention typically have a thermal stability which is at least comparable or even higher than that of bispecific antigen-binding molecules which have only one target binding domain but otherwise comprise a CD3 binding domain and, a half-life extending scFc domain, i.e. which are structurally less complex. The skilled person would expect that a more structurally complex protein-based molecule was less prone to thermal and other degradation, i.e. be less thermal stable. However, surprisingly the contrary is the case, a multitargeting bispecific antigen-binding molecule according to the present invention shows higher thermal stability, less monomer decrease after storage, higher monomer percentage after three freeze thaw cycles and higher protein homogeneity than a respective monotargeting bispecific antigen-binding molecule as disclosed herein.

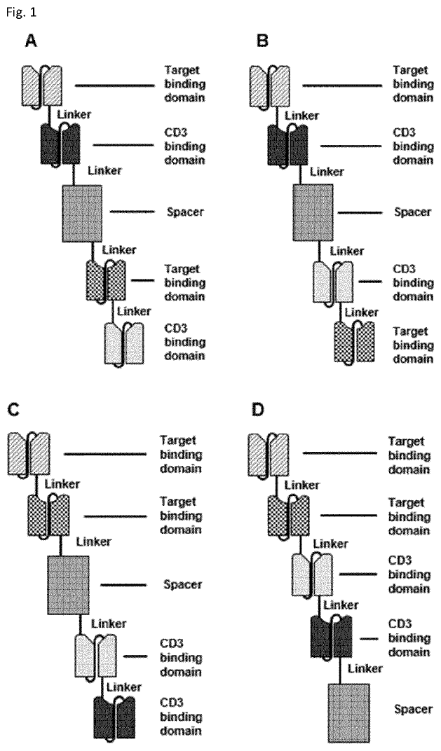

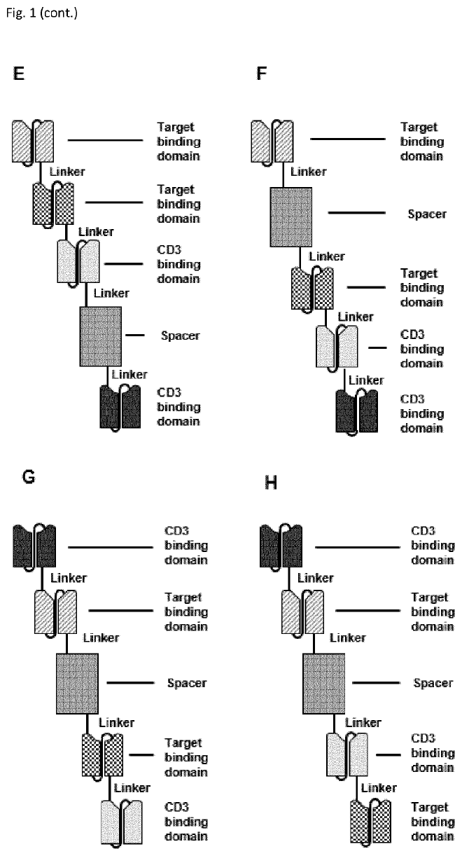

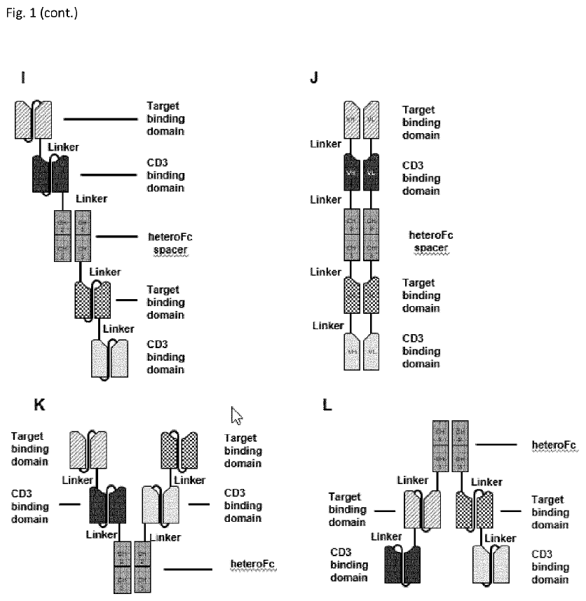

In an embodiment, the present invention provides a multitargeting bispecific antigen-binding molecule comprising all four such domains. In a preferred embodiment, the domains under (i.), (ii.), (iii.) and (iv.) are arranged in an N to C orientation (squared format, see FIG. 1A). However, alternatively, the multitargeting bispecific antigen-binding molecule may have the domains arranged in the order (i), (ii.), (iv) and (iii.) (mirror format, sec FIG. 1B), or (ii.), (i.), (iii.) and (iv.) or (ii.), (i.), (iv) and (iii.) in an N to C orientation. Surprisingly, all arrangements which (a.) either separate the target and the effector binder of any of the two bispecific entities or (b.) bring the two bispecific entities as such too close together will lead to constructs which show reduced ability for avidity effects in terms of a preferred selectivity gap as described herein between mono and dual positive target cells (sec FIGS. 1C to F and K and L (the latter in “V” and “A” shape)).

The term “polypeptide” is understood herein as an organic polymer which comprises at least one continuous, unbranched amino acid chain. In the context of the present invention, a polypeptide comprising more than one amino acid chain is likewise envisaged. An amino acid chain of a polypeptide typically comprises at least 50 amino acids, preferably at least 100, 200, 300, 400 or 500 amino acids. It is also envisaged in the context of the present invention that an amino acid chain of a polymer is linked to an entity which is not composed of amino acids.

The term “antigen-binding polypeptide” according to the present invention is preferably a polypeptide which immuno-specifically binds to its target or antigen. It typically comprises the heavy chain variable region (VH) and/or the light chain variable region (VL) of an antibody, or comprises domains derived therefrom. A polypeptide according to the invention comprises the minimum structural requirements of an antibody which allow for immuno-specific target binding. This minimum requirement may e.g. be defined by the presence of at least three light chain CDRs (i.e. CDR1, CDR2 and CDR3 of the VL region) and/or three heavy chain CDRs (i.e. CDR1, CDR2 and CDR3 of the VH region), preferably of all six CDRs. An antigen-binding molecule of the present invention is preferably a T-cell engaging polypeptide which may hence be characterized by the presence of three or six CDRs in either one or both binding domains, and the skilled person knows where (in which order) those CDRs are located within the binding domain. Preferably, an “antigen-binding molecule” is understood as an “antigen-binding polypeptide” in the context of the present invention. In an alternative embodiment, an antigen-binding polypeptide of the present invention may be an aptamer.

Alternatively, a molecule in the context of the present invention, is an antigen-binding polypeptide which corresponds to an “antibody construct” which typically refers to a molecule in which the structure and/or function is/are based on the structure and/or function of an antibody, e.g., of a full-length or whole immunoglobulin molecule. An antigen-binding molecule is hence capable of binding to its specific target or antigen and/or is/are drawn from the variable heavy chain (VH) and/or variable light chain (VL) domains of an antibody or fragment thereof. Furthermore, the domain which binds to its binding partner according to the present invention is understood herein as a binding domain of an antigen-binding molecule according to the invention. Typically, a binding domain according to the present invention comprises the minimum structural requirements of an antibody which allow for the target binding. This minimum requirement may e.g. be defined by the presence of at least the three light chain CDRs (i.e. CDR1, CDR2 and CDR3 of the VL region) and/or the three heavy chain CDRs (i.e. CDR1, CDR2 and CDR3 of the VH region), preferably of all six CDRs. An alternative approach to define the minimal structure requirements of an antibody is the definition of the epitope of the antibody within the structure of the specific target, respectively, the protein domain of the target protein composing the epitope region (epitope cluster) or by reference to a specific antibody competing with the epitope of the defined antibody. The antibodies on which the constructs according to the invention are based include for example monoclonal, recombinant, chimeric, deimmunized, humanized and human antibodies.

In the context of the present invention, a polypeptide of the present invention binds to its respective target structure in a particular manner. Preferably, a polypeptide according to the present invention comprises one paratope per binding domain which specifically or immuno-specifically binds to”, “(specifically or immuno-specifically) recognizes”, or “(specifically or immuno-specifically) reacts with” its respective target structure. This means in accordance with this invention that a polypeptide or a binding domain thereof interacts or (immuno-)specifically interacts with a given epitope on the target molecule (antigen) and CD3, respectively. This interaction or association occurs more frequently, more rapidly, with greater duration, with greater affinity, or with some combination of these parameters, to an epitope on the specific target than to alternative substances (non-target molecules). Because of the sequence similarity between homologous proteins in different species, a binding domain that (immuno-) specifically binds to its target (such as a human target) may, however, cross-react with homologous target molecules from different species (such as, from non-human primates). The term “specific/immuno-specific binding” can hence include the binding of a binding domain to epitopes and/or structurally related epitopes in more than one species. The term “(immuno-) selectively binds” does exclude the binding to structurally related epitopes.

The binding domain of an antigen-binding molecule according to the invention may e.g. comprise the above referred groups of CDRs. Preferably, those CDRs are comprised in the framework of an antibody light chain variable region (VL) and an antibody heavy chain variable region (VH); however, it does not have to comprise both. Fd fragments, for example, have two VH regions and often retain some antigen-binding function of the intact antigen-binding domain. Additional examples for the format of antibody fragments, antibody variants or binding domains include (1) a Fab fragment, a monovalent fragment having the VL. VH, CL and CH1 domains; (2) a F(ab′)2 fragment, a bivalent fragment having two Fab fragments linked by a disulfide bridge at the hinge region; (3) an Fd fragment having the two VH and CH1 domains; (4) an Fv fragment having the VL and VH domains of a single arm of an antibody, (5) a dAb fragment (Ward et al., (1989) Nature 341:544-546), which has a VH domain; (6) an isolated complementarity determining region (CDR), and (7) a single chain Fv (scFv), the latter being preferred (for example, derived from an scFV-library). Examples for embodiments of antigen-binding molecules according to the invention are e.g. described in WO 00/006605, WO 2005/040220, WO 2008/119567, WO 2010/037838, WO 2013/026837, WO 2013/026833, US 2014/0308285, US 2014/0302037, WO 2014/144722. WO 2014/151910, and WO 2015/048272.

Also, within the definition of “binding domain” or “domain which binds” are fragments of full-length antibodies, such as VH, VHH, VL, (s)dAb, Fv, Fd, Fab, Fab′, F(ab′)2 or “r IgG” (“half antibody”). Antigen-binding molecules according to the invention may also comprise modified fragments of antibodies, also called antibody variants, such as scFv, di-scFv or bi(s)-scFv, scFv-Fc, scFv-zipper, scFab, Fab2, Fab3, diabodies, single chain diabodies, tandem diabodies (Tandab's), tandem di-scFv, tandem tri-scFv, “multibodies” such as triabodies or tetrabodies, and single domain antibodies such as nanobodies or single variable domain antibodies comprising merely one variable domain, which may be VHH, VH or VL, that specifically bind an antigen or epitope independently of other V regions or domains. Typically, a binding domain of the present invention comprises a paratope which facilitates the binding to its binding partner.

As used herein, the terms “single-chain Fv.” “single-chain antibodies” or “scFv” refer to single polypeptide chain antibody fragments that comprise the variable regions from both the heavy and light chains, but lack the constant regions. Generally, a single-chain antibody further comprises a polypeptide linker between the VH and VL domains which enables it to form the desired structure which would allow for antigen binding. Single chain antibodies are discussed in detail by Pluckthun in The Pharmacology of Monoclonal Antibodies, vol. 113, Rosenburg and Moore eds. Springer-Verlag, New York, pp. 269-315 (1994). Various methods of generating single chain antibodies are known, including those described in U.S. Pat. Nos. 4,694,778 and 5,260,203; International Patent Application Publication No. WO 88/01649; Bird (1988) Science 242:423-442; Huston et al. (1988) Proc. Natl. Acad. Sci. USA 85:5879-5883; Ward et al. (1989) Nature 334:54454; Skerra et al. (1988) Science 242:1038-1041. In specific embodiments, single-chain antibodies can also be bispecific, multispecific, human, and/or humanized and/or synthetic.

In the context of the present invention, a paratope is understood as an antigen-binding site which is a part of a polypeptide as described herein and which recognizes and binds to an antigen. A paratope is typically a small region of about at least 5 amino acids. A paratope as understood herein typically comprises parts of antibody-derived heavy (VH) and light chain (VL) sequences. Each binding domain of a molecule according to the present invention is provided with a paratope comprising a set of 6 complementarity-determining regions (CDR loops) with three of each being comprised within the antibody-derived VH and VL sequence, respectively.

Furthermore, the definition of the term “antigen-binding molecule” includes preferably polyvalent/multivalent constructs and, thus, bispecific molecules, wherein bispecific means that they specifically bind to two cell types comprising distinctive antigenic structures, i.e. target cell(s) and effector cell(s). As the antigen-binding molecules of the present invention are preferably multitargeting, they are typically as well as polyvalent/multivalent molecules, i.e. they specifically bind more than two antigenic structures, preferably four distinct binding domains in the context of the present invention which are two target binding domains and two CD3 binding domains. The term “multitargeting bispecific antigen-binding molecule” comprises the terms “multitargeting bispecific T-cell engager molecule” and “multitargeting bispecific T-cell engager polypeptide (MBiTEP)”. A preferred “multitargeting bispecific antigen-binding molecule” is a “multitargeting bispecific T-cell engager molecule” or a “multitargeting bispecific T-cell engager polypeptide (MBiTEP)”. The term multitargeting bispecific T-cell engager molecule” is understood to comprise the term “multitargeting bispecific T-cell engager polypeptide. Moreover, the definition of the term “antigen-binding molecule” includes molecules comprising only one polypeptide chain as well as molecules consisting of more than one polypeptide chain, which chains can be either identical (homodimers, homotrimers or homo oligomers) or different (heterodimer, heterotrimer or heterooligomer). Such molecules comprising more than one polypeptide chain, i.e. typically two chains, have these chains typically attached to each other as heterodimers via charged pair binding, e.g. within a heteroFc entity which serves as a spacer and half-life extending moiety in between the two bispecific entities as described herein. Examples for the above identified antigen-binding molecules, e.g. antibody-based molecules and variants or derivatives thereof are described inter alia in Harlow and Lane, Antibodies a laboratory manual, CSHL Press (1988) and Using Antibodies: a laboratory manual, CSHL Press (1999), Kontermann and Dubel, Antibody Engineering, Springer, 2nd ed. 2010 and Little, Recombinant Antibodies for Immunotherapy, Cambridge University Press 2009.

The term “bispecific” as used herein refers to an antigen-binding molecule which is “at least bispecific”, i.e., it addresses two different cell types, i.e. target and effector cells, and comprises at least a first and third binding domain and a second and fourth binding domain, wherein at least two binding domains bind to two antigens or targets selected preferably from CD20, CD22, FLT3, MSLN, CDH3, CLL1 and EpCAM, and the other two binding domains of the same molecule bind to another antigen (here: CD3) on an effector cell, typically on a T cell. Accordingly, antigen-binding molecules according to the invention comprise specificities for at least two different antigens or targets. For example, two domains do preferably not bind to an extracellular epitope of CD3e of one or more of the species as described herein.

The term “target cell surface antigen” refers to an antigenic structure expressed by a cell and which is present at the cell surface such that it is accessible for an antigen-binding molecule as described herein. A preferred target cell surface antigen in the context of the present invention is a tumor associated antigen (TAA). It may be a protein, preferably the extracellular portion of a protein, or a carbohydrate structure, preferably a carbohydrate structure of a protein, such as a glycoprotein. It is preferably a tumor antigen. The term “bispecific antigen-binding molecule” of the invention also encompasses bispecific multitargeting antigen-binding molecules such as tritargeting antigen-binding molecules, the latter ones including three binding domains, or constructs having more than three (e.g. four, five . . . ) specificities.

Preferred in the context of the present invention is a molecule which is “multitargeting”, which is understood herein to be “at least targeting two targets (e.g. TAAs) per molecule of the invention typically per target cell”. In this regard, a multitargeting molecule such as an antigen-binding molecule is specific for two—typically identical—effector structures on an effector cell such as CD3, more preferably CD3epsilon (CD3e, which is comprised whenever reference is made to the “CD3” in the present invention), and at least two target cell surface antigens. Said specificity is conferred by respective binding domains as defined herein. Typically, “multitargeting” refers to a molecule which is specific for at least two (preferably different) target cell surface antigens (e.g. TAAs) which confers preferred properties of a multitargeting antigen-binding molecule according to the present invention, namely mitigation of antigen loss and increase of selectivity, i.e. selectivity for killing target cells which co-express the targets for which the molecule of the invention has binding domains and which target cells are associated with a disease. Thereby, the therapeutic window of the molecule of the invention is increased with respect to monotargeting bispecific molecules which typically leads to higher drug tolerability as demonstrated herein.

A T-cell engaging antigen-binding molecule, e.g. a single chain polypeptide, according to the present invention is preferably bispecific which is understood herein to typically comprise one domain binding to at least one target antigen and another domain binding to CD3. Hence, it does not occur naturally, and it is markedly different in its function from naturally occurring products. A polypeptide in accordance with the invention is hence an artificial “hybrid” polypeptide comprising at least two distinct binding domains with different specificities and is, thus, bispecific. Bispecific antigen-binding molecules can be produced by a variety of methods including fusion of hybridomas or linking of Fab′ fragments. See, e.g., Songsivilai & Lachmann, Clin. Exp. Immunol. 79:315-321 (1990).

The at least four binding domains and the variable domains (VH/VL) of the antigen-binding molecule of the present invention typically comprise peptide linkers (spacer peptides). The term “peptide linker” comprises in accordance with the present invention an amino acid sequence by which the amino acid sequences of one (variable and/or binding) domain and another (variable and/or binding) domain of the antigen-binding molecule of the invention are linked with each other. The peptide linker between the first and the second binding domain and the third and the fourth domain, wherein the first and the third domain are preferably capable to bind simultaneously to two targets, which are preferably different targets (e.g. TAA1 and TAA2) preferably on the same cell, are preferably flexible and of limited length, e.g. of 5, 6, 7, 8, 9, 10, 11, 12, 13, 14, 15, 16, 17 or 18 amino acids. The peptide linkers can also be used to fuse the spacer to the other domains of the antigen-binding molecule of the invention. An essential technical feature of such peptide linker is that it does not comprise any polymerization activity. Among the suitable peptide linkers are those described in U.S. Pat. Nos. 4,751,180 and 4,935,233 or WO 88/09344. The peptide linkers can also be used to attach other domains or modules or regions (such as half-life extending domains) to the antigen-binding molecule of the invention. However, typically the linker between the first and the second target binding domain differs from the intra-binder linker which links the VH and VL within the target binding domain. Said difference is the linker between the first and the second binding domain having one amino acid more than intra-binder linkers, e.g. six and five amino acids, respectively, such as SGGGGS versus GGGGS. This confers surprisingly flexibility and stability at the same time in the specific antigen-binding molecule format as described herein. The spacer (or synonymously spacer entity) between the two bispecific entities as described herein is a specific embodiment of a linker because a spacer also functions as a linker because it contributes to linking the two bispecific entities to preferably build at least one continuous polypeptide chain comprising the four binding domains or parts thereof. However, in addition, the spacer functions as an entity which spaces the two bispecific entities sterically apart. Accordingly, a spacer in the context of the present invention is a specific embodiment of a linker which—together with two further short and flexible linkers on each end—contributes to linking the two binding domains (of two different bispecific entities) but first and foremost spaces them apart in such a way that the two bispecific entities can advantageously act as described herein, e.g. show a surprisingly high selectivity gap.

The antigen-binding molecules of the present invention are preferably “in vitro generated antigen-binding molecules”. This term refers to an antigen-binding molecule according to the above definition where all or part of the variable region (e.g., at least one CDR) is generated in a non-immune cell selection, e.g., an in vitro phage display, protein chip or any other method in which candidate sequences can be tested for their ability to bind to an antigen. This term thus preferably excludes sequences generated solely by genomic rearrangement in an immune cell in an animal. A “recombinant antibody” is an antibody made through the use of recombinant DNA technology or genetic engineering.

The term “monoclonal antibody” (mAb) or monoclonal antibody from which an antigen-binding molecule as used herein is derived refers to an antibody obtained from a population of substantially homogeneous antibodies, i.e., the individual antibodies comprising the population are identical except for possible naturally occurring mutations and/or post-translation modifications (e.g., isomerizations, amidations) that may be present in minor amounts. Monoclonal antibodies are highly specific, being directed against a single antigenic side or determinant on the antigen, in contrast to conventional (polyclonal) antibody preparations which typically include different antibodies directed against different determinants (or epitopes). In addition to their specificity, the monoclonal antibodies are advantageous in that they are synthesized by the hybridoma culture, hence uncontaminated by other immunoglobulins. The modifier “monoclonal” indicates the character of the antibody as being obtained from a substantially homogeneous population of antibodies, and is not to be construed as requiring production of the antibody by any particular method.

For the preparation of monoclonal antibodies, any technique providing antibodies produced by continuous cell line cultures can be used. For example, monoclonal antibodies to be used may be made by the hybridoma method first described by Koehler et al., Nature, 256: 495 (1975), or may be made by recombinant DNA methods (see, e.g., U.S. Pat. No. 4,816,567). Examples for further techniques to produce human monoclonal antibodies include the trioma technique, the human B-cell hybridoma technique (Kozbor, Immunology Today 4 (1983), 72) and the EBV-hybridoma technique (Cole et al., Monoclonal Antibodies and Cancer Therapy, Alan R. Liss, Inc. (1985), 77-96).

Hybridomas can then be screened using standard methods, such as enzyme-linked immunosorbent assay (ELISA) and surface plasmon resonance analysis, e.g. Biacore™ to identify one or more hybridomas that produce an antibody that specifically binds with a specified antigen. Any form of the relevant antigen may be used as the immunogen, e.g., recombinant antigen, naturally occurring forms, any variants or fragments thereof, as well as an antigenic peptide thereof. Surface plasmon resonance as employed in the Biacore system can be used to increase the efficiency of phage antibodies which bind to an epitope of a target cell surface antigen (Schier, Human Antibodies Hybridomas 7 (1996), 97-105; Malmborg, J. Immunol. Methods 183 (1995), 7-13).

Another exemplary method of making monoclonal antibodies includes screening protein expression libraries, e.g., phage display or ribosome display libraries. Phage display is described, for example, in Ladner et al., U.S. Pat. No. 5,223,409; Smith (1985) Science 228:1315-1317, Clackson et al., Nature, 352: 624-628 (1991) and Marks et al., J. Mol. Biol., 222: 581-597 (1991).

In addition to the use of display libraries, the relevant antigen can be used to immunize a non-human animal, e.g., a rodent (such as a mouse, hamster, rabbit or rat). In one embodiment, the non-human animal includes at least a part of a human immunoglobulin gene. For example, it is possible to engineer mouse strains deficient in mouse antibody production with large fragments of the human Ig (immunoglobulin) loci. Using the hybridoma technology, antigen-specific monoclonal antibodies derived from the genes with the desired specificity may be produced and selected. See, e.g., XENOMOUSE™, Green et al. (1994) Nature Genetics 7:13-21, US 2003-0070185, WO 96/34096, and WO 96/33735.

A monoclonal antibody can also be obtained from a non-human animal, and then modified. e.g., humanized, deimmunized, rendered chimeric etc., using recombinant DNA techniques known in the art. Examples of modified antigen-binding molecules include humanized variants of non-human antibodies, “affinity matured” antibodies (sec, e.g. Hawkins et al. J. Mol. Biol. 254, 889-896 (1992) and Lowman et al., Biochemistry 30, 10832-10837 (1991)) and antibody mutants with altered effector function(s) (see, e.g., U.S. Pat. No. 5,648,260, Kontermann and Dübel (2010), loc. cit. and Little (2009), loc. cit.).

In immunology, affinity maturation is the process by which B cells produce antibodies with increased affinity for antigen during the course of an immune response. With repeated exposures to the same antigen, a host will produce antibodies of successively greater affinities. Like the natural prototype, the in vitro affinity maturation is based on the principles of mutation and selection. The in vitro affinity maturation has successfully been used to optimize antibodies, antigen-binding molecules, and antibody fragments. Random mutations inside the CDRs are introduced using radiation, chemical mutagens or error-prone PCR. In addition, the genetic diversity can be increased by chain shuffling. Two or three rounds of mutation and selection using display methods like phage display usually results in antibody fragments with affinities in the low nanomolar range.

A preferred type of an amino acid substitutional variation of the antigen-binding molecules involves substituting one or more hypervariable region residues of a parent antibody (e. g. a humanized or human antibody). Generally, the resulting variant(s) selected for further development will have improved biological properties relative to the parent antibody from which they are generated. A convenient way for generating such substitutional variants involves affinity maturation using phage display. Briefly, several hypervariable region sides (e. g. 6-7 sides) are mutated to generate all possible amino acid substitutions at each side. The antibody variants thus generated are displayed in a monovalent fashion from filamentous phage particles as fusions to the gene III product of M13 packaged within each particle. The phage-displayed variants are then screened for their biological activity (e. g. binding affinity) as herein disclosed. In order to identify candidate hypervariable region sides for modification, alanine scanning mutagenesis can be performed to identify hypervariable region residues contributing significantly to antigen binding. Alternatively, or additionally, it may be beneficial to analyze a crystal structure of the antigen-antibody complex to identify contact points between the binding domain and, e.g., human CS1, BCMA, CD20, CD22. FLT3, CD123, CDH3, MSLN, CLL1 or EpCAM. Such contact residues and neighbouring residues are candidates for substitution according to the techniques elaborated herein. Once such variants are generated, the panel of variants is subjected to screening as described herein and antibodies with superior properties in one or more relevant assays may be selected for further development.

The monoclonal antibodies and antigen-binding molecules of the present invention specifically include “chimeric” antibodies (immunoglobulins) in which a portion of the heavy and/or light chain is identical with or homologous to corresponding sequences in antibodies derived from a particular species or belonging to a particular antibody class or subclass, while the remainder of the chain(s) is/are identical with or homologous to corresponding sequences in antibodies derived from another species or belonging to another antibody class or subclass, as well as fragments of such antibodies, so long as they exhibit the desired biological activity (U.S. Pat. No. 4,816,567; Morrison et al., Proc. Natl. Acad. Sci. USA, 81: 6851-6855 (1984)). Chimeric antibodies of interest herein include “primitized” antibodies comprising variable domain antigen-binding sequences derived from a non-human primate (e.g., Old World Monkey, Ape etc.) and human constant region sequences. A variety of approaches for making chimeric antibodies have been described. Sec e.g., Morrison et al., Proc. Natl. Acad. ScL U.S.A. 81:6851, 1985; Takeda et al., Nature 314:452, 1985, Cabilly et al., U.S. Pat. No. 4,816,567; Boss et al., U.S. Pat. No. 4,816,397; Tanaguchi et al., EP 0171496; EP 0173494; and GB 2177096.

An antibody, antigen-binding molecule, antibody fragment or antibody variant may also be modified by specific deletion of human T cell epitopes (a method called “deimmunization”) by the methods disclosed for example in WO 98/52976 or WO 00/34317. Briefly, the heavy and light chain variable domains of an antibody can be analyzed for peptides that bind to MHC class II; these peptides represent potential T cell epitopes (as defined in WO 98/52976 and WO 00/34317). For detection of potential T cell epitopes, a computer modeling approach termed “peptide threading” can be applied, and in addition a database of human MHC class II binding peptides can be searched for motifs present in the VH and VL sequences, as described in WO 98/52976 and WO 00/34317. These motifs bind to any of the 18 major MHC class II DR allotypes, and thus constitute potential T cell epitopes. Potential T cell epitopes detected can be eliminated by substituting small numbers of amino acid residues in the variable domains, or preferably, by single amino acid substitutions. Typically, conservative substitutions are made. Often, but not exclusively, an amino acid common to a position in human germline antibody sequences may be used. Human germline sequences are disclosed e.g. in Tomlinson, et al. (1992) J. Mol. Biol. 227:776-798; Cook, G. P. et al. (1995) Immunol. Today Vol. 16 (5): 237-242; and Tomlinson et al. (1995) EMBO J. 14: 14:4628-4638. The V BASE directory provides a comprehensive directory of human immunoglobulin variable region sequences (compiled by Tomlinson, L A. et al. MRC Centre for Protein Engineering, Cambridge, UK). These sequences can be used as a source of human sequence, e.g., for framework regions and CDRs. Consensus human framework regions can also be used, for example as described in U.S. Pat. No. 6,300,064.

“Humanized” antibodies, antigen-binding molecules, variants or fragments thereof (such as Fv, Fab, Fab′, F(ab′)2 or other antigen-binding subsequences of antibodies) are antibodies or immunoglobulins of mostly human sequences, which contain (a) minimal sequence(s) derived from non-human immunoglobulin. For the most part, humanized antibodies are human immunoglobulins (recipient antibody) in which residues from a hypervariable region (also CDR) of the recipient are replaced by residues from a hypervariable region of a non-human (e.g., rodent) species (donor antibody) such as mouse, rat, hamster or rabbit having the desired specificity, affinity, and capacity. In some instances, Fv framework region (FR) residues of the human immunoglobulin are replaced by corresponding non-human residues. Furthermore, “humanized antibodies” as used herein may also comprise residues which are found neither in the recipient antibody nor the donor antibody. These modifications are made to further refine and optimize antibody performance. The humanized antibody may also comprise at least a portion of an immunoglobulin constant region (Fc), typically that of a human immunoglobulin. For further details, see Jones et al., Nature, 321: 522-525 (1986); Reichmann et al., Nature, 332: 323-329 (1988); and Presta, Curr. Op. Struct. Biol., 2: 593-596 (1992).

Humanized antibodies or fragments thereof can be generated by replacing sequences of the Fv variable domain that are not directly involved in antigen binding with equivalent sequences from human Fv variable domains. Exemplary methods for generating humanized antibodies or fragments thereof are provided by Morrison (1985) Science 229:1202-1207; by Oi et al. (1986) BioTechniques 4:214; and by U.S. Pat. Nos. 5,585,089; 5,693,761; 5,693,762; 5,859,205; and 6,407,213. Those methods include isolating, manipulating, and expressing the nucleic acid sequences that encode all or part of immunoglobulin Fv variable domains from at least one of a heavy or light chain. Such nucleic acids may be obtained from a hybridoma producing an antibody against a predetermined target, as described above, as well as from other sources. The recombinant DNA encoding the humanized antibody molecule can then be cloned into an appropriate expression vector.

Humanized antibodies may also be produced using transgenic animals such as mice that express human heavy and light chain genes, but are incapable of expressing the endogenous mouse immunoglobulin heavy and light chain genes. Winter describes an exemplary CDR grafting method that may be used to prepare the humanized antibodies described herein (U.S. Pat. No. 5,225,539). All of the CDRs of a particular human antibody may be replaced with at least a portion of a non-human CDR, or only some of the CDRs may be replaced with non-human CDRs. It is only necessary to replace the number of CDRs required for binding of the humanized antibody to a predetermined antigen.

A humanized antibody can be optimized by the introduction of conservative substitutions, consensus sequence substitutions, germline substitutions and/or back mutations. Such altered immunoglobulin molecules can be made by any of several techniques known in the art. (e.g., Teng et al., Proc. Natl. Acad. Sci. U.S.A., 80: 7308-7312, 1983; Kozbor et al., Immunology Today, 4: 7279, 1983; Olsson et al., Meth. Enzymol., 92: 3-16, 1982, and EP 239 400).

The term “human antibody”, “human antigen-binding molecule” and “human binding domain” includes antibodies, antigen-binding molecules and binding domains having antibody regions such as variable and constant regions or domains which correspond substantially to human germline immunoglobulin sequences known in the art, including, for example, those described by Kabat et al. (1991) (loc. cit.). The human antibodies, antigen-binding molecules or binding domains of the invention may include amino acid residues not encoded by human germline immunoglobulin sequences (e.g., mutations introduced by random or side-specific mutagenesis in vitro or by somatic mutation in vivo), for example in the CDRs, and in particular, in CDR3. The human antibodies, antigen-binding molecules or binding domains can have at least one, two, three, four, five, or more positions replaced with an amino acid residue that is not encoded by the human germline immunoglobulin sequence. The definition of human antibodies, antigen-binding molecules and binding domains as used herein also contemplates fully human antibodies, which include only non-artificially and/or genetically altered human sequences of antibodies as those can be derived by using technologies or systems such as the Xenomouse. Preferably, a “fully human antibody” does not include amino acid residues not encoded by human germline immunoglobulin sequences.

In some embodiments, the antigen-binding molecules of the invention are “isolated” or “substantially pure” antigen-binding molecules. “Isolated” or “substantially pure”, when used to describe the antigen-binding molecules disclosed herein, means an antigen-binding molecule that has been identified, separated and/or recovered from a component of its production environment. Preferably, the antigen-binding molecule is free or substantially free of association with all other components from its production environment. Contaminant components of its production environment, such as that resulting from recombinant transfected cells, are materials that would typically interfere with diagnostic or therapeutic uses for the polypeptide, and may include enzymes, hormones, and other proteinaceous or non-proteinaceous solutes. The antigen-binding molecules may e.g. constitute at least about 5%, or at least about 50% by weight of the total protein in a given sample. It is understood that the isolated protein may constitute from 5% to 99.9% by weight of the total protein content, depending on the circumstances. The polypeptide may be made at a significantly higher concentration through the use of an inducible promoter or high expression promoter, such that it is made at increased concentration levels. The definition includes the production of an antigen-binding molecule in a wide variety of organisms and/or host cells that are known in the art. In preferred embodiments, the antigen-binding molecule will be purified (1) to a degree sufficient to obtain at least 15 residues of N-terminal or internal amino acid sequence by use of a spinning cup sequenator, or (2) to homogeneity by SDS-PAGE under non-reducing or reducing conditions using Coomassie blue or, preferably, silver stain. Ordinarily, however, an isolated antigen-binding molecule will be prepared by at least one purification step.

The term “binding domain” characterizes in connection with the present invention a domain which (specifically) binds to/interacts with/recognizes a given target epitope or a given target side on the target molecules (antigens), e.g. CS1, BCMA, CD20, CD22, FLT3, CD123, CLL1, MSLN, or EpCAM, and CD3, respectively. The structure and function of the typically first and third or second and fourth binding domain (recognizing e.g. CS1, BCMA, CD20, CD22, FLT3, CD123, CLL1, MSLN, or EpCAM), and preferably also the structure and/or function of the effector binding domain (typically the second and fourth or first and third binding domain recognizing CD3), is/are based on the structure and/or function of an antibody, e.g. of a full-length or whole immunoglobulin molecule, and/or is/are drawn from the variable heavy chain (VH) and/or variable light chain (VL) domains of an antibody or fragment thereof. Preferably the target cell surface antigen(s) binding domain(s) is/are characterized by the presence of three light chain CDRs (i.e. CDR1, CDR2 and CDR3 of the VL region) and/or three heavy chain CDRs (i.e. CDR1, CDR2 and CDR3 of the VH region). The effector (typically CD3) binding domain preferably also comprises the minimum structural requirements of an antibody which allow for the target binding. More preferably, the second binding domain comprises at least three light chain CDRs (i.e. CDR1, CDR2 and CDR3 of the VL region) and/or three heavy chain CDRs (i.e. CDR1, CDR2 and CDR3 of the VH region). It is envisaged that the first and/or second binding domain is produced by or obtainable by phage-display or library screening methods rather than by grafting CDR sequences from a pre-existing (monoclonal) antibody into a scaffold.

According to the present invention, binding domains are in the form of one or more polypeptides. Such polypeptides may include proteinaceous parts and non-proteinaceous parts (e.g. chemical linkers or chemical cross-linking agents such as glutaraldehyde). Proteins (including fragments thereof, preferably biologically active fragments, and peptides, usually having less than 30 amino acids) comprise two or more amino acids coupled to each other via a covalent peptide bond (resulting in a chain of amino acids).

The term “polypeptide” as used herein describes a group of molecules, which usually consist of more than 30 amino acids. Polypeptides may further form multimers such as dimers, trimers and higher oligomers, i.e., consisting of more than one polypeptide molecule. Polypeptide molecules forming such dimers, trimers etc. may be identical or non-identical. The corresponding higher order structures of such multimers are, consequently, termed homo- or heterodimers, homo- or heterotrimers etc. An example for a heteromultimer is an antibody molecule, which, in its naturally occurring form, consists of two identical light polypeptide chains and two identical heavy polypeptide chains. The terms “peptide”. “polypeptide” and “protein” also refer to naturally modified peptides/polypeptides/proteins wherein the modification is effected e.g. by post-translational modifications like glycosylation, acetylation, phosphorylation and the like. A “peptide”, “polypeptide” or “protein” when referred to herein may also be chemically modified such as pegylated. Such modifications are well known in the art and described herein below.

Preferably the binding domains which binds to any of CS1, BCMA, CD20, CD22, FLT3, CD123, CLL1, CDH3, MSLN, and EpCAM, and/or the binding domains which binds to CD3ε is/are human binding domains. Antibodies and antigen-binding molecules comprising at least one human binding domain avoid some of the problems associated with antibodies or antigen-binding molecules that possess non-human such as rodent (e.g. murine, rat, hamster or rabbit) variable and/or constant regions. The presence of such rodent derived proteins can lead to the rapid clearance of the antibodies or antigen-binding molecules or can lead to the generation of an immune response against the antibody or antigen-binding molecule by a patient. In order to avoid the use of rodent derived antibodies or antigen-binding molecules, human or fully human antibodies/antigen-binding molecules can be generated through the introduction of human antibody function into a rodent so that the rodent produces fully human antibodies.

The ability to clone and reconstruct megabase-sized human loci in yeast artificial chromosomes YACs and to introduce them into the mouse germline provides a powerful approach to elucidating the functional components of very large or crudely mapped loci as well as generating useful models of human disease. Furthermore, the use of such technology for substitution of mouse loci with their human equivalents could provide unique insights into the expression and regulation of human gene products during development, their communication with other systems, and their involvement in disease induction and progression.

An important practical application of such a strategy is the “humanization” of the mouse humoral immune system. Introduction of human immunoglobulin (Ig) loci into mice in which the endogenous Ig genes have been inactivated offers the opportunity to study the mechanisms underlying programmed expression and assembly of antibodies as well as their role in B-cell development. Furthermore, such a strategy could provide an ideal source for production of fully human monoclonal antibodies (mAbs)—an important milestone towards fulfilling the promise of antibody therapy in human disease. Fully human antibodies or antigen-binding molecules are expected to minimize the immunogenic and allergic responses intrinsic to mouse or mouse-derivatized mAbs and thus to increase the efficacy and safety of the administered antibodies/antigen-binding molecules. The use of fully human antibodies or antigen-binding molecules can be expected to provide a substantial advantage in the treatment of chronic and recurring human diseases, such as inflammation, autoimmunity, and cancer, which require repeated compound administrations.

One approach towards this goal was to engineer mouse strains deficient in mouse antibody production with large fragments of the human Ig loci in anticipation that such mice would produce a large repertoire of human antibodies in the absence of mouse antibodies. Large human Ig fragments would preserve the large variable gene diversity as well as the proper regulation of antibody production and expression. By exploiting the mouse machinery for antibody diversification and selection and the lack of immunological tolerance to human proteins, the reproduced human antibody repertoire in these mouse strains should yield high affinity antibodies against any antigen of interest, including human antigens. Using the hybridoma technology, antigen-specific human mAbs with the desired specificity could be readily produced and selected. This general strategy was demonstrated in connection with the generation of the first XenoMouse mouse strains (see Green et al. Nature Genetics 7:13-21 (1994)). The XenoMouse strains were engineered with YACs containing 245 kb and 190 kb-sized germline configuration fragments of the human heavy chain locus and kappa light chain locus, respectively, which contained core variable and constant region sequences. The human Ig containing YACs proved to be compatible with the mouse system for both rearrangement and expression of antibodies and were capable of substituting for the inactivated mouse Ig genes. This was demonstrated by their ability to induce B cell development, to produce an adult-like human repertoire of fully human antibodies, and to generate antigen-specific human mAbs. These results also suggested that introduction of larger portions of the human Ig loci containing greater numbers of V genes, additional regulatory elements, and human Ig constant regions may recapitulate substantially the full repertoire that is characteristic of the human humoral response to infection and immunization. The work of Green et al. was recently extended to the introduction of greater than approximately 80% of the human antibody repertoire through introduction of megabase sized, germline configuration YAC fragments of the human heavy chain loci and kappa light chain loci, respectively. See Mendez et al. Nature Genetics 15:146-156 (1997) and U.S. patent application Ser. No. 08/759,620.

The production of the XenoMouseanimals is further discussed and delineated in U.S. patent application Ser. No. 07/466,008, Ser. No. 07/610,515, Ser. No. 07/919,297, Ser. No. 07/922,649, Ser. No. 08/031,801, Ser. No. 08/112,848, Ser. No. 08/234,145, Ser. No. 08/376,279, Ser. No. 08/430,938, Ser. No. 08/464,584, Ser. No. 08/464,582, Ser. No. 08/463,191, Ser. No. 08/462,837, Ser. No. 08/486,853, Ser. No. 08/486,857, Ser. No. 08/486,859, Ser. No. 08/462,513, Ser. No. 08/724,752, and Ser. No. 08/759,620; and U.S. Pat. Nos. 6,162,963; 6,150,584; 6,114,598; 6,075,181, and 5,939,598 and Japanese Patent Nos. 3 068 180 B2, 3 068 506 B2, and 3 068 507 B2. See also Mendez et al. Nature Genetics 15:146-156 (1997) and Green and Jakobovits J. Exp. Med. 188:483-495 (1998), EP 0 463 151 B1, WO 94/02602. WO 96/34096, WO 98/24893, WO 00/76310, and WO 03/47336.

In an alternative approach, others, including GenPharm International, Inc., have utilized a “minilocus” approach. In the minilocus approach, an exogenous Ig locus is mimicked through the inclusion of pieces (individual genes) from the Ig locus. Thus, one or more VH genes, one or more DH genes, one or more JH genes, a mu constant region, and a second constant region (preferably a gamma constant region) are formed into a construct for insertion into an animal. This approach is described in U.S. Pat. No. 5,545,807 to Surani et al. and U.S. Pat. Nos. 5,545,806; 5,625,825; 5,625,126; 5,633,425; 5,661,016; 5,770,429; 5,789,650; 5,814,318; 5,877,397; 5,874,299; and 6,255,458 each to Lonberg and Kay, U.S. Pat. Nos. 5,591,669 and 6,023,010 to Krimpenfort and Berns, U.S. Pat. Nos. 5,612,205; 5,721,367; 5,789,215 to Berns et al., and U.S. Pat. No. 5,643,763 to Choi and Dunn, and GenPharm International U.S. patent application Ser. No. 07/574,748, Ser. No. 07/575,962, Ser. No. 07/810,279, Ser. No. 07/853,408, Ser. No. 07/904,068, Ser. No. 07/990,860, Ser. No. 08/053,131, Ser. No. 08/096,762, Ser. No. 08/155,301, Ser. No. 08/161,739, Ser. No. 08/165,699, Ser. No. 08/209,741. See also EP 0 546 073 B1, WO 92/03918, WO 92/22645, WO 92/22647, WO 92/22670, WO 93/12227, WO 94/00569, WO 94/25585, WO 96/14436, WO 97/13852, and WO 98/24884 and U.S. Pat. No. 5,981,175. See further Taylor et al. (1992), Chen et al. (1993), Tuaillon et al. (1993), Choi et al. (1993), Lonberg et al. (1994), Taylor et al. (1994), and Tuaillon et al. (1995), Fishwild et al. (1996).

Kirin has also demonstrated the generation of human antibodies from mice in which, through microcell fusion, large pieces of chromosomes, or entire chromosomes, have been introduced. See European Patent Application Nos. 773 288 and 843 961. Xenerex Biosciences is developing a technology for the potential generation of human antibodies. In this technology, SCID mice are reconstituted with human lymphatic cells, e.g., B and/or T cells. Mice are then immunized with an antigen and can generate an immune response against the antigen. See U.S. Pat. Nos. 5,476,996; 5,698,767; and 5,958,765.

Human anti-mouse antibody (HAMA) responses have led the industry to prepare chimeric or otherwise humanized antibodies. It is however expected that certain human anti-chimeric antibody (HACA) responses will be observed, particularly in chronic or multi-dose utilizations of the antibody. Thus, it would be desirable to provide antigen-binding molecules comprising a human binding domain against CS1, BCMA, CD20, CD22, FLT3, CD123, CLL1, CDH3, MSLN, or EpCAM and a human binding domain against CD3ε in order to vitiate concerns and/or effects of HAMA or HACA response.

The terms “(specifically) binds to”, (specifically) recognizes”, “is (specifically) directed to”, and “(specifically) reacts with” mean in accordance with this invention that a binding domain interacts or specifically interacts with a given epitope or a given target side on the target molecules (antigens), here: CS1, BCMA, CD20, CD22, FLT3, CD123, CLL1, CDH3, MSLN, or EpCAM, and CD3ε as effector, respectively.

The term “epitope” refers to a side on an antigen to which a binding domain, such as an antibody or immunoglobulin, or a derivative, fragment or variant of an antibody or an immunoglobulin, specifically binds. An “epitope” is antigenic and thus the term epitope is sometimes also referred to herein as “antigenic structure” or “antigenic determinant”. Thus, the binding domain is an “antigen interaction side”. Said binding/interaction is also understood to define a “specific recognition”.

“Epitopes” can be formed both by contiguous amino acids or non-contiguous amino acids juxtaposed by tertiary folding of a protein. A “linear epitope” is an epitope where an amino acid primary sequence comprises the recognized epitope. A linear epitope typically includes at least 3 or at least 4, and more usually, at least 5 or at least 6 or at least 7, for example, about 8 to about 10 amino acids in a unique sequence.

A “conformational epitope”, in contrast to a linear epitope, is an epitope wherein the primary sequence of the amino acids comprising the epitope is not the sole defining component of the epitope recognized (e.g., an epitope wherein the primary sequence of amino acids is not necessarily recognized by the binding domain). Typically, a conformational epitope comprises an increased number of amino acids relative to a linear epitope. With regard to recognition of conformational epitopes, the binding domain recognizes a three-dimensional structure of the antigen, preferably a peptide or protein or fragment thereof (in the context of the present invention, the antigenic structure for one of the binding domains is comprised within the target cell surface antigen protein). For example, when a protein molecule folds to form a three-dimensional structure, certain amino acids and/or the polypeptide backbone forming the conformational epitope become juxtaposed enabling the antibody to recognize the epitope. Methods of determining the conformation of epitopes include, but are not limited to, x-ray crystallography, two-dimensional nuclear magnetic resonance (2D-NMR) spectroscopy and site-directed spin labelling and electron paramagnetic resonance (EPR) spectroscopy.

A method for epitope mapping is described in the following: When a region (a contiguous amino acid stretch) in the human CS1, BCMA, CD20, CD22, FLT3, CD123, CLL1, CDH3, MSLN, or EpCAM protein is exchanged or replaced with its corresponding region of a non-human and non-primate CS1, BCMA, CD20, CD22, FLT3, CD123, CLL1, CDH3, MSLN, or EpCAM (e.g., mouse CS1. BCMA, CD20, CD22, FLT3, CD123, CLL1, CDH3, MSLN, or EpCAM, but others like chicken, rat, hamster, rabbit etc. may also be conceivable), a decrease in the binding of the binding domain is expected to occur, unless the binding domain is cross-reactive for the non-human, non-primate CS1, BCMA, CD20, CD22, FLT3, CD123, CLL1, CDH3, MSLN, or EpCAM used. Said decrease is preferably at least 10%, 20%, 30%, 40%, or 50%; more preferably at least 60%, 70%, or 80%, and most preferably 90%, 95% or even 100% in comparison to the binding to the respective region in the human CS1, BCMA, CD20, CD22, FLT3, CD123, CLL1, CDH3, MSLN, or EpCAM protein, whereby binding to the respective region in the human CS1, BCMA, CD20, CD22, FLT3, CD123, CLL1, CDH3, MSLN, or EpCAM protein is set to be 100%. It is envisaged that the aforementioned human CS1, BCMA, CD20, CD22, FLT3, CD123, CLL1, CDH3, MSLN, or EpCAM/non-human CS1, BCMA, CD20, CD22, FLT3, CD123, CLL1, CDH3, MSLN, or EpCAM chimeras are expressed in CHO cells. It is also envisaged that the human CS1, BCMA, CD20, CD22, FLT3, CD123, CLL1, CDH3, MSLN, or EpCAM/non-human CS1, BCMA, CD20, CD22, FLT3, CD123, CLL1, CDH3, MSLN, or EpCAM chimeras are fused with a transmembrane domain and/or cytoplasmic domain of a different membrane-bound protein such as EpCAM.

In an alternative or additional method for epitope mapping, several truncated versions of the human CS1, BCMA, CD20, CD22, FLT3, CD123, CLL1, CDH3, MSLN, or EpCAM extracellular domain can be generated in order to determine a specific region that is recognized by a binding domain. In these truncated versions, the different extracellular CS1, BCMA, CD20, CD22, FLT3, CD123, CLL1, CDH3, MSLN, or EpCAM domains/sub-domains or regions are stepwise deleted, starting from the N-terminus. It is envisaged that the truncated CS1, BCMA, CD20, CD22, FLT3, CD123, CLL1, CDH3, MSLN, or EpCAM versions may be expressed in CHO cells. It is also envisaged that the truncated CS1, BCMA, CD20, CD22, FLT3, CD123, CLL1, CDH3, MSLN, or EpCAM versions may be fused with a transmembrane domain and/or cytoplasmic domain of a different membrane-bound protein such as EpCAM. It is also envisaged that the truncated CS1. BCMA, CD20, CD22, FLT3, CD123, CLL1, CDH3, MSLN, or EpCAM versions may encompass a signal peptide domain at their N-terminus, for example a signal peptide derived from mouse IgG heavy chain signal peptide. It is furthermore envisaged that the truncated CS1, BCMA, CD20, CD22, FLT3, CD123, CLL1, CDH3, MSLN, or EpCAM versions may encompass a v5 domain at their N-terminus (following the signal peptide) which allows verifying their correct expression on the cell surface. A decrease or a loss of binding is expected to occur with those truncated CS1, BCMA, CD20, CD22, FLT3, CD123, CLL1, CDH3, MSLN, or EpCAM versions which do not encompass any more the CS1, BCMA, CD20, CD22, FLT3, CD123, CLL1, CDH3, MSLN, or EpCAM region that is recognized by the binding domain. The decrease of binding is preferably at least 10%, 20%, 30%, 40%, 50%; more preferably at least 60%, 70%, 80%, and most preferably 90%, 95% or even 100%, whereby binding to the entire human CS1, BCMA, CD20, CD22, FLT3, CD123, CLL1, CDH3, MSLN, or EpCAM protein (or its extracellular region or domain) is set to be 100.

A further method to determine the contribution of a specific residue of CS1, BCMA, CD20, CD22, FLT3, CD123, CLL1, CDH3, MSLN, or EpCAM to the recognition by an antigen-binding molecule or binding domain is alanine scanning (see e.g. Morrison K L & Weiss G A. Cur Opin Chem Biol. 2001 June;5(3):302-7), where each residue to be analyzed is replaced by alanine, e.g. via site-directed mutagenesis. Alanine is used because of its non-bulky, chemically inert, methyl functional group that nevertheless mimics the secondary structure references that many of the other amino acids possess. Sometimes bulky amino acids such as valine or leucine can be used in cases where conservation of the size of mutated residues is desired. Alanine scanning is a mature technology which has been used for a long period of time.

The interaction between the binding domain and the epitope or the region comprising the epitope implies that a binding domain exhibits appreciable affinity for the epitope/the region comprising the epitope on a particular protein or antigen (here: CD20, CD22, FLT3, CD123, CLL1, CDH3, MSLN, or EpCAM and CD3, respectively) and, generally, does not exhibit significant reactivity with proteins or antigens other than the CS1, BCMA, CD20, CD22, FLT3, CD123, CLL1, CDH3, MSLN, or EpCAM or CD3. “Appreciable affinity” includes binding with an affinity of about 10⁻⁶ M (KD) or stronger. Preferably, binding is considered specific when the binding affinity is about 10⁻¹² to 10⁻⁸ M, 10⁻¹² to 10⁻⁹ M, 10⁻¹² to 10⁻¹⁰ M, 10⁻¹¹ to 10⁻⁸ M, preferably of about 10⁻¹¹ to 10⁻⁹ M. Whether a binding domain specifically reacts with or binds to a target can be tested readily by, inter alia, comparing the reaction of said binding domain with a target protein or antigen with the reaction of said binding domain with proteins or antigens other than the CS1, BCMA, CD20, CD22, FLT3, CD123, CLL1, CDH3, MSLN, or EpCAM or CD3. Preferably, a binding domain of the invention does not essentially or substantially bind to proteins or antigens other than CS1, BCMA, CD20, CD22, FLT3, CD123, CLL1, CDH3, MSLN, or EpCAM or CD3 (i.e., the first binding domain is not capable of binding to proteins other than CS1, BCMA, CD20, CD22, FLT3, CD123, CLL1, CDH3, MSLN, or EpCAM and the second binding domain is not capable of binding to proteins other than CD3). It is an envisaged characteristic of the antigen-binding molecules according to the present invention to have superior affinity characteristics in comparison to other HLE formats. Such a superior affinity, in consequence, suggests a prolonged half-life in vivo. The longer half-life of the antigen-binding molecules according to the present invention may reduce the duration and frequency of administration which typically contributes to improved patient compliance. This is of particular importance as the antigen-binding molecules of the present invention are particularly beneficial for highly weakened or even multimorbid cancer patients.

The term “does not essentially/substantially bind” or “is not capable of binding” means that a binding domain of the present invention does not bind a protein or antigen other than the CS1, BCMA. CD20, CD22, FLT3, CD123, CLL1, CDH3, MSLN, or EpCAM or CD3 as effector, i.e., does not show reactivity of more than 30%, preferably not more than 20%, more preferably not more than 10%, particularly preferably not more than 9%, 8%, 7%, 6% or 5% with proteins or antigens other than CS1, BCMA, CD20, CD22, FLT3, CD123, CLL1, CDH3, MSLN, or EpCAM or CD3 as effector, whereby binding to the CS1, BCMA, CD20, CD22, FLT3, CD123, CLL1, CDH3, MSLN, or EpCAM or CD3 as effector, respectively, is set to be 100%.

Specific binding is believed to be effected by specific motifs in the amino acid sequence of the binding domain and the antigen. Thus, binding is achieved as a result of their primary, secondary and/or tertiary structure as well as the result of secondary modifications of said structures. The specific interaction of the antigen-interaction-side with its specific antigen may result in a simple binding of said side to the antigen. Moreover, the specific interaction of the antigen-interaction-side with its specific antigen may alternatively or additionally result in the initiation of a signal, e.g. due to the induction of a change of the conformation of the antigen, an oligomerization of the antigen, etc.

The term “variable” refers to the portions of the antibody or immunoglobulin domains that exhibit variability in their sequence and that are involved in determining the specificity and binding affinity of a particular antibody (i.e., the “variable domain(s)”). The pairing of a variable heavy chain (VH) and a variable light chain (VL) together forms a single antigen-binding site.

Variability is not evenly distributed throughout the variable domains of antibodies; it is concentrated in sub-domains of each of the heavy and light chain variable regions. These sub-domains are called “hypervariable regions” or “complementarity determining regions” (CDRs). The more conserved (i.e., non-hypervariable) portions of the variable domains are called the “framework” regions (FRM or FR) and provide a scaffold for the six CDRs in three dimensional space to form an antigen-binding surface. The variable domains of naturally occurring heavy and light chains each comprise four FRM regions (FR1, FR2, FR3, and FR4), largely adopting a β-sheet configuration, connected by three hypervariable regions, which form loops connecting, and in some cases forming part of, the β-sheet structure. The hypervariable regions in each chain are held together in close proximity by the FRM and, with the hypervariable regions from the other chain, contribute to the formation of the antigen-binding side (see Kabat et al., loc. cit.).

The terms “CDR”, and its plural “CDRs”, refer to the complementarity determining region of which three make up the binding character of a light chain variable region (CDR-L1, CDR-L2 and CDR-L3) and three make up the binding character of a heavy chain variable region (CDR-H1, CDR-H2 and CDR-H3). CDRs contain most of the residues responsible for specific interactions of the antibody with the antigen and hence contribute to the functional activity of an antibody molecule: they are the main determinants of antigen specificity.

The exact definitional CDR boundaries and lengths are subject to different classification and numbering systems. CDRs may therefore be referred to by Kabat, Chothia, contact or any other boundary definitions, including the numbering system described herein. Despite differing boundaries, each of these systems has some degree of overlap in what constitutes the so called “hypervariable regions” within the variable sequences. CDR definitions according to these systems may therefore differ in length and boundary areas with respect to the adjacent framework region. See for example Kabat (an approach based on cross-species sequence variability), Chothia (an approach based on crystallographic studies of antigen-antibody complexes), and/or MacCallum (Kabat et al., loc. cit.; Chothia et al., J. Mol. Biol, 1987, 196: 901-917; and MacCallum et al., J. Mol. Biol, 1996, 262: 732). Still another standard for characterizing the antigen binding side is the AbM definition used by Oxford Molecular's AbM antibody modeling software. See, e.g., Protein Sequence and Structure Analysis of Antibody Variable Domains. In: Antibody Engineering Lab Manual (Ed.: Duebel, S. and Kontermann, R., Springer-Verlag, Heidelberg). To the extent that two residue identification techniques define regions of overlapping, but not identical regions, they can be combined to define a hybrid CDR. However, the numbering in accordance with the so-called Kabat system is preferred.

Typically, CDRs form a loop structure that can be classified as a canonical structure. The term “canonical structure” refers to the main chain conformation that is adopted by the antigen binding (CDR) loops. From comparative structural studies, it has been found that five of the six antigen binding loops have only a limited repertoire of available conformations. Each canonical structure can be characterized by the torsion angles of the polypeptide backbone. Correspondent loops between antibodies may, therefore, have very similar three dimensional structures, despite high amino acid sequence variability in most parts of the loops (Chothia and Lesk, J. Mol. Biol., 1987, 196: 901; Chothia et al., Nature, 1989, 342: 877; Martin and Thornton, J. Mol. Biol, 1996, 263: 800). Furthermore, there is a relationship between the adopted loop structure and the amino acid sequences surrounding it. The conformation of a particular canonical class is determined by the length of the loop and the amino acid residues residing at key positions within the loop, as well as within the conserved framework (i.e., outside of the loop). Assignment to a particular canonical class can therefore be made based on the presence of these key amino acid residues.

The term “canonical structure” may also include considerations as to the linear sequence of the antibody, for example, as catalogued by Kabat (Kabat et al., loc. cit.). The Kabat numbering scheme (system) is a widely adopted standard for numbering the amino acid residues of an antibody variable domain in a consistent manner and is the preferred scheme applied in the present invention as also mentioned elsewhere herein. Additional structural considerations can also be used to determine the canonical structure of an antibody. For example, those differences not fully reflected by Kabat numbering can be described by the numbering system of Chothia et al. and/or revealed by other techniques, for example, crystallography and two- or three-dimensional computational modeling. Accordingly, a given antibody sequence may be placed into a canonical class which allows for, among other things, identifying appropriate chassis sequences (e.g., based on a desire to include a variety of canonical structures in a library). Kabat numbering of antibody amino acid sequences and structural considerations as described by Chothia et al., loc. cit. and their implications for construing canonical aspects of antibody structure, are described in the literature. The subunit structures and three-dimensional configurations of different classes of immunoglobulins are well known in the art. For a review of the antibody structure, see Antibodies: A Laboratory Manual, Cold Spring Harbor Laboratory, eds. Harlow et al., 1988.

The CDR3 of the light chain and, particularly, the CDR3 of the heavy chain may constitute the most important determinants in antigen binding within the light and heavy chain variable regions. In some antigen-binding molecules, the heavy chain CDR3 appears to constitute the major area of contact between the antigen and the antibody. In vitro selection schemes in which CDR3 alone is varied can be used to vary the binding properties of an antibody or determine which residues contribute to the binding of an antigen. Hence, CDR3 is typically the greatest source of molecular diversity within the antibody-binding side. H3, for example, can be as short as two amino acid residues or greater than 26 amino acids.

In a classical full-length antibody or immunoglobulin, each light (L) chain is linked to a heavy (H) chain by one covalent disulfide bond, while the two H chains are linked to each other by one or more disulfide bonds depending on the H chain isotype. The CH domain most proximal to VH is usually designated as CH1. The constant (“C”) domains are not directly involved in antigen binding, but exhibit various effector functions, such as antibody-dependent, cell-mediated cytotoxicity and complement activation. The Fc region of an antibody is comprised within the heavy chain constant domains and is for example able to interact with cell surface located Fc receptors.

The sequence of antibody genes after assembly and somatic mutation is highly varied, and these varied genes are estimated to encode 1010 different antibody molecules (Immunoglobulin Genes, 2^{nd} ed., eds. Jonio et al., Academic Press, San Diego, C A. 1995). Accordingly, the immune system provides a repertoire of immunoglobulins. The term “repertoire” refers to at least one nucleotide sequence derived wholly or partially from at least one sequence encoding at least one immunoglobulin. The sequence(s) may be generated by rearrangement in vivo of the V. D, and J segments of heavy chains, and the V and J segments of light chains. Alternatively, the sequence(s) can be generated from a cell in response to which rearrangement occurs, e.g., in vitro stimulation. Alternatively, part or all of the sequence(s) may be obtained by DNA splicing, nucleotide synthesis, mutagenesis, and other methods, see, e.g., U.S. Pat. No. 5,565,332. A repertoire may include only one sequence or may include a plurality of sequences, including ones in a genetically diverse collection.

The term “Fc portion” or “Fc monomer” means in connection with this invention a polypeptide comprising at least one domain having the function of a CH2 domain and at least one domain having the function of a CH3 domain of an immunoglobulin molecule. As apparent from the term “Fc monomer”, the polypeptide comprising those CH domains is a “polypeptide monomer”. An Fc monomer can be a polypeptide comprising at least a fragment of the constant region of an immunoglobulin excluding the first constant region immunoglobulin domain of the heavy chain (CH1), but maintaining at least a functional part of one CH2 domain and a functional part of one CH3 domain, wherein the CH2 domain is amino terminal to the CH3 domain. In a preferred aspect of this definition, an Fc monomer can be a polypeptide constant region comprising a portion of the Ig-Fc hinge region, a CH2 region and a CH3 region, wherein the hinge region is amino terminal to the CH2 domain. It is envisaged that the hinge region of the present invention promotes dimerization. Such Fc polypeptide molecules can be obtained by papain digestion of an immunoglobulin region (of course resulting in a dimer of two Fc polypeptide), for example and not limitation. In another aspect of this definition, an Fc monomer can be a polypeptide region comprising a portion of a CH2 region and a CH3 region. Such Fc polypeptide molecules can be obtained by pepsin digestion of an immunoglobulin molecule, for example and not limitation. In one embodiment, the polypeptide sequence of an Fc monomer is substantially similar to an Fc polypeptide sequence of: an IgG Fc region, an IgG₂ Fc region, an IgG₃ Fc region, an IgG₄ Fc region, an IgM Fc region, an IgA Fc region, an IgD Fc region and an IgE Fc region. (See, e.g., Padlan, Molecular Immunology, 31(3), 169-217 (1993)). Because there is some variation between immunoglobulins, and solely for clarity, Fc monomer refers to the last two heavy chain constant region immunoglobulin domains of IgA, IgD, and IgG, and the last three heavy chain constant region immunoglobulin domains of IgE and IgM. As mentioned, the Fc monomer can also include the flexible hinge N-terminal to these domains. For IgA and IgM, the Fc monomer may include the J chain. For IgG, the Fc portion comprises immunoglobulin domains CH2 and CH3 and the hinge between the first two domains and CH2. Although the boundaries of the Fc portion may vary an example for a human IgG heavy chain Fc portion comprising a functional hinge, CH2 and CH3 domain can be defined e.g. to comprise residues D231 (of the hinge domain—corresponding to D234 in Table 1 below) to P476, respectively L476 (for IgG₄) of the carboxyl-terminus of the CH3 domain, wherein the numbering is according to Kabat. The two Fc portion or Fc monomer, which are fused to each other via a peptide linker are a preferred example of the spacer between the two bispecific entities of the antigen-binding molecule of the invention, which may also be defined as scFc domain.

In one embodiment of the invention it is envisaged that a scFc domain as disclosed herein, respectively the Fc monomers fused to each other are comprised only in the spacer of the antigen-binding molecule.

In line with the present invention an IgG hinge region can be identified by analogy using the Kabat numbering as set forth in Table 1. In line with the above, it is envisaged that for a hinge domain/region of the present invention the minimal requirement comprises the amino acid residues corresponding to the IgG1 sequence stretch of D231 D234 to P243 according to the Kabat numbering. It is likewise envisaged that a hinge domain/region of the present invention comprises or consists of the IgG1 hinge sequence DKTHTCPPCP (SEQ ID NO: 330) (corresponding to the stretch D234 to P243 as shown in Table 1 below—variations of said sequence are also envisaged provided that the hinge region still promotes dimerization). In a preferred embodiment of the invention the glycosylation site at Kabat position 314 of the CH2 domains in the spacer of the antigen-binding molecule is removed by a N314X substitution, wherein X is any amino acid excluding Q. Said substitution is preferably a N314G substitution. In a more preferred embodiment, said CH2 domain additionally comprises the following substitutions (position according to Kabat) V321C and R309C (these substitutions introduce the intra domain cysteine disulfide bridge at Kabat positions 309 and 321).

It is also envisaged that the spacer of the antigen-binding molecule of the invention is a scFc domain which comprises or consists in an amino to carboxyl order: DKTHTCPPCP (SEQ ID NO: 330) (i.e. hinge) -CH2-CH3-linker-DKTHTCPPCP (SEQ ID NO: 330) (i.e. hinge) -CH2-CH3. The peptide linker of the aforementioned antigen-binding molecule is in a preferred embodiment characterized by the amino acid sequence Gly-Gly-Gly-Gly-Ser, i.e. Gly4Ser (SEQ ID NO: 7), or polymers thereof, i.e. (Gly4Ser)x, where x is an integer of 5 or greater (e.g. 5, 6, 7, 8 etc. or greater), 6 being preferred ((Gly4Ser)6). Said construct may further comprise the aforementioned substitutions: N314X, preferably N314G, and/or the further substitutions V321C and R309C. In a preferred embodiment of the antigen-binding molecules of the invention as defined herein before, it is envisaged that the second domain binds to an extracellular epitope of the human and/or the Macaca CD3ε chain.

TABLE 1
Kabat numbering of the amino acid residues of the hinge region

| IMGT | | |
|---|---|---|
| numbering for | IgG₁ amino acid | Kabat |
| the hinge | translation | numbering |
| 1 | (E) | 226 |
| 2 | P | 227 |
| 3 | K | 228 |
| 4 | S | 232 |
| 5 | C | 233 |
| 6 | D | 234 |
| 7 | K | 235 |
| 8 | T | 236 |
| 9 | H | 237 |
| 10 | T | 238 |
| 11 | C | 239 |
| 12 | P | 240 |
| 13 | P | 241 |
| 14 | C | 242 |
| 15 | P | 243 |

In further embodiments of the present invention, the hinge domain/region comprises or consists of the IgG2 subtype hinge sequence ERKCCVECPPCP (SEQ ID NO: 331), the IgG3 subtype hinge sequence ELKTPLDTTHTCPRCP (SEQ ID NO: 332) or ELKTPLGDTTHTCPRCP (SEQ ID NO:333), and/or the IgG4 subtype hinge sequence ESKYGPPCPSCP (SEQ ID NO: 444). The IgG1 subtype hinge sequence may be the following one EPKSCDKTHTCPPCP (as shown in Table 1 and SEQ ID NO: 445). These core hinge regions are thus also envisaged in the context of the present invention.

The location and sequence of the IgG CH2 and IgG CD3 domain can be identified by analogy using the Kabat numbering as set forth in Table 2:

TABLE 2
Kabat numbering of the amino acid residues
of the IgG CH2 and CH3 region

| IgG | CH2 aa | CH2 Kabat | CH3 aa | CH3 Kabat |
|---|---|---|---|---|
| subtype | translation | numbering | translation | numbering |
| IgG₁ | APE . . . KAK | 244 . . . 360 | GQP . . . PGK | 361 . . . 478 |
| IgG₂ | APP . . . KTK | 244 . . . 360 | GQP . . . PGK | 361 . . . 478 |
| IgG₃ | APE . . . KTK | 244 . . . 360 | GQP . . . PGK | 361 . . . 478 |
| IgG₄ | APE . . . KAK | 244 . . . 360 | GQP . . . LGK | 361 . . . 478 |

In one embodiment of the invention the emphasized bold amino acid residues in the CH3 domain of the first or both Fc monomers are deleted.

The peptide linker, by whom the polypeptide monomers (“Fc portion” or “Fc monomer”) of the spacer are fused to each other, preferably comprises at least 25 amino acid residues (25, 26, 27, 28, 29, 30 etc.). More preferably, this peptide linker comprises at least 30 amino acid residues (30, 31, 32, 33, 34, 35 etc.). It is also preferred that the linker comprises up to 40 amino acid residues, more preferably up to 35 amino acid residues, most preferably exactly 30 amino acid residues. A preferred embodiment of such peptide linker is characterized by the amino acid sequence Gly-Gly-Gly-Gly-Ser, i.e. Gly₄Ser (SEQ ID NO: 7), or polymers thereof, i.e. (Gly₄Ser)x, where x is an integer of 5 or greater (e.g. 6, 7 or 8). Preferably the integer is 6 or 7, more preferably the integer is 6.

In the event that a linker is used to fuse the first domain to the second domain, and/or the third to the fourth domain, and/or the second and the third domain to the spacer, this linker is preferably of a length and sequence sufficient to ensure that each of the first and second domains can, independently from one another, retain their differential binding specificities. For peptide linkers which connect the at least two binding domains (or two variable domains) in the antigen-binding molecule of the invention, those peptide linkers are preferred which comprise only a few number of amino acid residues, e.g. 12 amino acid residues or less. Thus, peptide linkers of 12, 11, 10, 9, 8, 7, 6 or 5 amino acid residues are preferred. An envisaged peptide linker with less than 5 amino acids comprises 4, 3, 2 or one amino acid(s), wherein Gly-rich linkers are preferred. A preferred embodiment of the peptide linker for a fusion the first and the second domain is depicted in SEQ ID NO:1. A preferred linker embodiment of the peptide linker for fusing the second and the third domain to the spacer is a (Gly)₄-linker, also called G₄-linker.

A particularly preferred “single” amino acid in the context of one of the above described “peptide linker” is Gly. Accordingly, said peptide linker may consist of the single amino acid Gly. In a preferred embodiment of the invention a peptide linker is characterized by the amino acid sequence Gly-Gly-Gly-Gly-Ser, i.e. Gly₄Ser (SEQ ID NO: 1), or polymers thereof, i.e. (Gly₄Ser)x, where x is an integer of 1 or greater (e.g. 2 or 3). Preferred linkers are depicted in SEQ ID NOs: 1 to 12. The characteristics of said peptide linker, which comprise the absence of the promotion of secondary structures, are known in the art and are described e.g. in Dall'Acqua et al. (Biochem. (1998) 37, 9266-9273), Cheadle et al. (Mol Immunol (1992) 29, 21-30) and Raag and Whitlow (FASEB (1995) 9(1), 73-80). Peptide linkers which furthermore do not promote any secondary structures are preferred. The linkage of said domains to each other can be provided, e.g., by genetic engineering, as described in the examples. Methods for preparing fused and operatively linked bispecific single chain constructs and expressing them in mammalian cells or bacteria are well-known in the art (e.g. WO 99/54440 or Sambrook et al., Molecular Cloning: A Laboratory Manual, Cold Spring Harbor Laboratory Press, Cold Spring Harbor, New York, 2001).

In a preferred embodiment of the antigen-binding molecule or the present invention the first and second domain form an antigen-binding molecule in a format selected from the group consisting of (scFv)₂, scFv-single domain mAb, diabody and oligomers of any of these formats.

According to a particularly preferred embodiment, and as documented in the appended examples, the first and the second domain of the antigen-binding molecule of the invention is a “bispecific single chain antigen-binding molecule”, more preferably a bispecific “single chain Fv” (scFv). Although the two domains of the Fv fragment, VL and VH, are coded for by separate genes, they can be joined, using recombinant methods, by a synthetic linker—as described hereinbefore—that enables them to be made as a single protein chain in which the VL and VH regions pair to form a monovalent molecule; see e.g., Huston et al. (1988) Proc. Natl. Acad. Sci USA 85:5879-5883). These antibody fragments are obtained using conventional techniques known to those with skill in the art, and the fragments are evaluated for function in the same manner as are whole or full-length antibodies. A single-chain variable fragment (scFv) is hence a fusion protein of the variable region of the heavy chain (VH) and of the light chain (VL) of immunoglobulins, usually connected with a short linker peptide of about ten to about 25 amino acids, preferably about 15 to 20 amino acids. The linker is usually rich in glycine for flexibility, as well as serine or threonine for solubility, and can either connect the N-terminus of the VH with the C-terminus of the VL, or vice versa. This protein retains the specificity of the original immunoglobulin, despite removal of the constant regions and introduction of the linker.

Bispecific single chain antigen-binding molecules are known in the art and are described in WO 99/54440, Mack, J. Immunol. (1997), 158, 3965-3970, Mack, PNAS, (1995), 92, 7021-7025, Kufer, Cancer Immunol. Immunother., (1997), 45, 193-197. Löffler, Blood, (2000), 95, 6, 2098-2103, Brühl, Immunol., (2001), 166, 2420-2426, Kipriyanov, J. Mol. Biol., (1999), 293, 41-56. Techniques described for the production of single chain antibodies (see, inter alia, U.S. Pat. No. 4,946,778, Kontermann and Dübel (2010), loc. cit. and Little (2009), loc. cit.) can be adapted to produce single chain antigen-binding molecules specifically recognizing (an) elected target(s).

Bivalent (also called divalent) or bispecific single-chain variable fragments (bi-scFvs or di-scFvs having the format (scFv)₂ can be engineered by linking two scFv molecules (e.g. with linkers as described hereinbefore). If these two scFv molecules have the same binding specificity, the resulting (scFv)₂ molecule will preferably be called bivalent (i.e. it has two valences for the same target epitope). If the two scFv molecules have different binding specificities, the resulting (scFv)₂ molecule will preferably be called bispecific. The linking can be done by producing a single peptide chain with two VH regions and two VL regions, yielding tandem scFvs (see e.g. Kufer P. et al., (2004) Trends in Biotechnology 22(5):238-244). Another possibility is the creation of scFv molecules with linker peptides that are too short for the two variable regions to fold together (e.g. about five amino acids), forcing the scFvs to dimerize. This type is known as diabodies (see e.g. Hollinger, Philipp et al., (July 1993) Proceedings of the National Academy of Sciences of the United States of America 90 (14): 6444-8).

In line with this invention either the first, the second or the first and the second domain may comprise a single domain antibody, respectively the variable domain or at least the CDRs of a single domain antibody. Single domain antibodies comprise merely one (monomeric) antibody variable domain which is able to bind selectively to a specific antigen, independently of other V regions or domains. The first single domain antibodies were engineered from heavy chain antibodies found in camelids, and these are called V_{H}H fragments. Cartilaginous fishes also have heavy chain antibodies (IgNAR) from which single domain antibodies called VNAR fragments can be obtained. An alternative approach is to split the dimeric variable domains from common immunoglobulins e.g. from humans or rodents into monomers, hence obtaining VH or VL as a single domain Ab. Although most research into single domain antibodies is currently based on heavy chain variable domains, nanobodies derived from light chains have also been shown to bind specifically to target epitopes. Examples of single domain antibodies are called sdAb, nanobodies or single variable domain antibodies.

A (single domain mAb)₂ is hence a monoclonal antigen-binding molecule composed of (at least) two single domain monoclonal antibodies, which are individually selected from the group comprising V_{H}, V_{L}, V_{H}H and V_{NAR}. The linker is preferably in the form of a peptide linker. Similarly, an “scFv-single domain mAb” is a monoclonal antigen-binding molecule composed of at least one single domain antibody as described above and one scFv molecule as described above. Again, the linker is preferably in the form of a peptide linker.

Whether or not an antigen-binding molecule competes for binding with another given antigen-binding molecule can be measured in a competition assay such as a competitive ELISA or a cell-based competition assay. Avidin-coupled microparticles (beads) can also be used. Similar to an avidin-coated ELISA plate, when reacted with a biotinylated protein, each of these beads can be used as a substrate on which an assay can be performed. Antigen is coated onto a bead and then precoated with the first antibody. The second antibody is added and any additional binding is determined. Possible means for the read-out includes flow cytometry.

T cells or T lymphocytes are a type of lymphocyte (itself a type of white blood cell) that play a central role in cell-mediated immunity. There are several subsets of T cells, each with a distinct function. T cells can be distinguished from other lymphocytes, such as B cells and NK cells, by the presence of a T cell receptor (TCR) on the cell surface. The TCR is responsible for recognizing antigens bound to major histocompatibility complex (MHC) molecules and is composed of two different protein chains. In 95% of the T cells, the TCR consists of an alpha (a) and beta (B) chain. When the TCR engages with antigenic peptide and MHC (peptide/MHC complex), the T lymphocyte is activated through a series of biochemical events mediated by associated enzymes, co-receptors, specialized adaptor molecules, and activated or released transcription factors.

The CD3 receptor complex is a protein complex and is composed of four chains. In mammals, the complex contains a CD3γ (gamma) chain, a CD3ε (delta) chain, and two CD3ε (epsilon) chains. These chains associate with the T cell receptor (TCR) and the so-called ((zeta) chain to form the T cell receptor CD3 complex and to generate an activation signal in T lymphocytes. The CD3γ (gamma), CD3δ (delta), and CD3ε (epsilon) chains are highly related cell-surface proteins of the immunoglobulin superfamily containing a single extracellular immunoglobulin domain. The intracellular tails of the CD3 molecules contain a single conserved motif known as an immunoreceptor tyrosine-based activation motif or ITAM for short, which is essential for the signaling capacity of the TCR. The CD3 epsilon molecule is a polypeptide which in humans is encoded by the CD3E gene which resides on chromosome 11. The most preferred epitope of CD3 epsilon is comprised within amino acid residues 1-27 of the human CD3 epsilon extracellular domain. It is envisaged that antigen-binding molecules according to the present invention typically and advantageously show less unspecific T cell activation, which is not desired in specific immunotherapy. This translates to a reduced risk of side effects.

The redirected lysis of target cells via the recruitment of T cells by a multitargeting least bispecific antigen-binding molecule involves cytolytic synapse formation and delivery of perforin and granzymes. The engaged T cells are capable of serial target cell lysis, and are not affected by immune escape mechanisms interfering with peptide antigen processing and presentation, or clonal T cell differentiation; see, for example, WO 2007/042261.

Cytotoxicity mediated by antigen-binding molecules of the invention can be measured in various ways. Effector cells can be e.g. stimulated enriched (human) CD8 positive T cells or unstimulated (human) peripheral blood mononuclear cells (PBMC). If the target cells are of macaque origin or express or are transfected with macaque CS1, BCMA, CD20, CD22, FLT3, CD123, CLL1, CHD3, MSLN, or EpCAM which is bound by the first domain, the effector cells should also be of macaque origin such as a macaque T cell line, e.g. 4119LnPx. The target cells should express (at least the extracellular domain of) CS1, BCMA, CD20, CD22, FLT3, CD123, CLL1, CHD3, MSLN, or EpCAM, e.g. human or macaque CS1, BCMA, CD20, CD22, FLT3, CD123, CLL1, CHD3, MSLN, or EpCAM. Target cells can be a cell line (such as CHO) which is stably or transiently transfected with CS1, BCMA, CD20, CD22, FLT3, CD123, CLL1, CHD3, MSLN, or EpCAM, e.g. human or macaque CS1, BCMA, CD20, CD22, FLT3, CD123, CLL1, CHD3, MSLN, or EpCAM. Usually EC₅₀ values are expected to be lower with target cell lines expressing higher levels of CS1. BCMA, CD20, CD22, FLT3, CD123, CLL1, CHD3, MSLN, or EpCAM on the cell surface. The effector to target cell (E:T) ratio is usually about 10:1, but can also vary. Cytotoxic activity of CS1, BCMA. CD20, CD22, FLT3, CD123, CLL1, CHD3, MSLN, or EpCAM bispecific antigen-binding molecules can be measured in a ⁵¹Cr-release assay (incubation time of about 18 hours) or in a in a FACS-based cytotoxicity assay (incubation time of about 48 hours). Modifications of the assay incubation time (cytotoxic reaction) are also possible. Other methods of measuring cytotoxicity are well-known to the skilled person and comprise MTT or MTS assays. ATP-based assays including bioluminescent assays, the sulforhodamine B (SRB) assay, WST assay, clonogenic assay and the ECIS technology.

The cytotoxic activity mediated by CS1, BCMA, CD20, CD22, FLT3, CD123, CLL1, CHD3, MSLN, or EpCAMxCD3 bispecific antigen-binding molecules of the present invention is preferably measured in a cell-based cytotoxicity assay. It may also be measured in a ⁵¹Cr-release assay. It is represented by the EC₅₀ value, which corresponds to the half maximal effective concentration (concentration of the antigen-binding molecule which induces a cytotoxic response halfway between the baseline and maximum). Preferably, the EC50 value of the CS1, BCMA, CD20, CD22, FLT3, CD123, CLL1, CHD3, MSLN, or EpCAMxCD3 bispecific antigen-binding molecules is ≤5000 μM or ≤4000 μM, more preferably ≤3000 μM or ≤2000 μM, even more preferably ≤1000 μM or ≤500 μM, even more preferably ≤400 μM or ≤300 μM, even more preferably ≤200 μM, even more preferably ≤100 μM, even more preferably ≤50 μM, even more preferably ≤20 μM or ≤10 μM, and most preferably ≤5 μM.

The above given EC50 values can be measured in different assays. The skilled person is aware that an EC50 value can be expected to be lower when stimulated/enriched CD8+ T cells are used as effector cells, compared with unstimulated PBMC. It can furthermore be expected that the EC50 values are lower when the target cells express a high number of CS1, BCMA, CD20, CD22, FLT3, CD123, CLL1, CHD3, MSLN, or EpCAM compared with a low target expression rat. For example, when stimulated/enriched human CD8+ T cells are used as effector cells (and either CS1, BCMA, CD20, CD22, FLT3, CD123, CLL1, CHD3, MSLN, or EpCAM transfected cells such as CHO cells or CS1, BCMA, CD20, CD22, FLT3, CD123, CLL1, CHD3, MSLN, or EpCAM positive human cell lines are used as target cells), the EC50 value of the CS1, BCMA, CD20, CD22, FLT3, CD123, CLL1, CHD3, MSLN, or EpCAMxCD3 bispecific antigen-binding molecule is preferably ≤1000 μM, more preferably ≤500 μM, even more preferably ≤250 μM, even more preferably ≤100 μM, even more preferably ≤50 μM, even more preferably ≤10 μM, and most preferably ≤5 μM. When human PBMCs are used as effector cells, the EC50 value of the CS1, BCMA, CD20, CD22, FLT3, CD123, CLL1, CHD3, MSLN, or EpCAMxCD3 bispecific antigen-binding molecule is preferably ≤5000 μM or ≤4000 μM (in particular when the target cells are CS1, BCMA, CD20, CD22, FLT3, CD123, CLL1, CHD3, MSLN, or EpCAM positive human cell lines), more preferably ≤2000 μM (in particular when the target cells are CS1, BCMA, CD20, CD22, FLT3, CD123, CLL1. CHD3, MSLN, or EpCAM transfected cells such as CHO cells), more preferably ≤1000 μM or ≤500 μM, even more preferably ≤200 μM, even more preferably ≤150 μM, even more preferably ≤100 μM, and most preferably ≤50 μM, or lower. When a macaque T cell line such as LnPx4119 is used as effector cells, and a macaque CS1, BCMA, CD20, CD22, FLT3, CD123, CLL1, CHD3, MSLN, or EpCAM transfected cell line such as CHO cells is used as target cell line, the EC₅₀ value of the CS1, BCMA, CD20, CD22, FLT3, CD123, CLL1, CHD3, MSLN, or EpCAMxCD3 bispecific antigen-binding molecule is preferably ≤2000 μM or ≤1500 μM, more preferably ≤1000 μM or ≤500 μM, even more preferably ≤300 μM or <250 μM, even more preferably ≤100 μM, and most preferably ≤50 μM.

Preferably, the CS1, BCMA, CD20, CD22, FLT3, CD123, CLL1, CHD3, MSLN, or EpCAMxCD3 bispecific antigen-binding molecules of the present invention do not induce/mediate lysis or do not essentially induce/mediate lysis of CS1, BCMA, CD20, CD22, FLT3, CD123, CLL1, CHD3, MSLN, or EpCAM negative cells such as CHO cells. The term “do not induce lysis”, “do not essentially induce lysis”, “do not mediate lysis” or “do not essentially mediate lysis” means that an antigen-binding molecule of the present invention does not induce or mediate lysis of more than 30%, preferably not more than 20%, more preferably not more than 10%, particularly preferably not more than 9%, 8%, 7%, 6% or 5% of CS1, BCMA, CD20, CD22, FLT3, CD123, CLL1, CHD3, MSLN, or EpCAM negative cells, whereby lysis of a CS1, BCMA, CD20, CD22, FLT3, CD123, CLL1, CHD3, MSLN, or EpCAM positive human cell line is set to be 100%. This usually applies for concentrations of the antigen-binding molecule of up to 500 nM. The skilled person knows how to measure cell lysis without further ado. Moreover, the present specification teaches specific instructions how to measure cell lysis.

The difference in cytotoxic activity between the monomeric and the dimeric isoform of individual CS1, BCMA, CD20, CD22, FLT3, CD123, CLL1, CHD3, MSLN, or EpCAMxCD3 bispecific antigen-binding molecules is referred to as “potency gap”. This potency gap can e.g. be calculated as ratio between EC₅₀ values of the molecule's monomeric and dimeric form. Potency gaps of the CS1, BCMA, CD20, CD22, FLT3, CD123, CLL1, CHD3, MSLN, or EpCAMxCD3 bispecific antigen-binding molecules of the present invention are preferably ≤5, more preferably ≤4, even more preferably ≤3, even more preferably ≤2 and most preferably ≤1.

The first, second, third and/or the fourth binding domain of the antigen-binding molecule of the invention is/are preferably cross-species specific for members of the mammalian order of primates. Cross-species specific CD3 binding domains are, for example, those described herein and in WO 2008/119567. According to one embodiment, the first and third binding domain, in addition to binding to human CS1, BCMA, CD20, CD22, FLT3, CD123, CLL1, CHD3, MSLN, or EpCAM and human CD3, respectively, will also bind to CS1, BCMA, CD20, CD22, FLT3, CD123, CLL1, CHD3, MSLN, or EpCAM/CD3 of primates including (but not limited to) new world primates (such as Callithrix jacchus, Saguinus Oedipus or Saimiri sciureus), old world primates (such baboons and macaques), gibbons, and non-human homininae.

In one embodiment of the antigen-binding molecule of the invention the first domain binds to human CS1, BCMA, CD20, CD22, FLT3, CD123, CLL1, CHD3, MSLN, or EpCAM and further binds to macaque CS1, BCMA, CD20, CD22, FLT3, CD123, CLL1, CHD3, MSLN, or EpCAM, such as CS1, BCMA, CD20, CD22, FLT3, CD123, CLL1, CHD3, MSLN, or EpCAM of Macaca fascicularis, and more preferably, to macaque CS1, BCMA, CD20, CD22, FLT3, CD123, CLL1. CHD3, MSLN, or EpCAM expressed on the surface of cells, e.g. such as CHO or 293 cells. The affinity of the first domain for CS1, BCMA, CD20, CD22, FLT3, CD123, CLL1, CHD3, MSLN, or EpCAM, preferably for human CS1, BCMA, CD20, CD22, FLT3, CD123, CLL1, CHD3, MSLN, or EpCAM, is preferably ≤100 nM or ≤50 nM, more preferably ≤25 nM or ≤20 nM, more preferably ≤15 nM or ≤10 nM, even more preferably ≤5 nM, even more preferably ≤2.5 nM or ≤2 nM, even more preferably ≤1 nM, even more preferably <0.6 nM, even more preferably ≤0.5 nM, and most preferably ≤0.4 nM. The affinity can be measured for example in a BIAcore assay or in a Scatchard assay. Other methods of determining the affinity are also well-known to the skilled person. The affinity of the first domain for macaque CS1, BCMA, CD20, CD22, FLT3, CD123, CLL1, CHD3, MSLN, or EpCAM is preferably ≤15 nM, more preferably ≤10 nM, even more preferably ≤5 nM, even more preferably ≤1 nM, even more preferably ≤0.5 nM, even more preferably ≤0.1 nM, and most preferably ≤0.05 nM or even ≤0.01 nM.

Preferably the affinity gap of the antigen-binding molecules according to the invention for binding macaque CS1, BCMA, CD20, CD22, FLT3, CD123, CLL1, CHD3, MSLN, or EpCAM versus human CS1, BCMA, CD20, CD22, FLT3, CD123, CLL1, CHD3, MSLN, or EpCAM [ma CS1, BCMA, CD20, CD22, FLT3, CD123, CLL1, CHD3, MSLN, or EpCAM: hu CS1, BCMA, CD20, CD22, FLT3, CD123, CLL1, CHD3, MSLN, or EpCAM (as determined e.g. by surface plasmon resonance analysis such as BiaCore™ or by Scatchard analysis) is <100, preferably <20, more preferably <15, further preferably <10, even more preferably <8, more preferably <6 and most preferably <2. Preferred ranges for the affinity gap of the antigen-binding molecules according to the invention for binding macaque CS1, BCMA, CD20, CD22, FLT3, CD123, CLL1, CHD3, MSLN, or EpCAM versus human CS1, BCMA, CD20, CD22, FLT3, CD123, CLL1, CHD3, MSLN, or EpCAM are between 0.1 and 20, more preferably between 0.2 and 10, even more preferably between 0.3 and 6, even more preferably between 0.5 and 3 or between 0.5 and 2.5, and most preferably between 0.5 and 2 or between 0.6 and 2.

The second and the fourth binding domain of the antigen-binding molecule of the invention typically binds to human CD3 epsilon and/or to Macaca CD3 epsilon. In a preferred embodiment, where a selectivity gap is achieved, the second and the fourth binding domain, or alternatively, the first and the third binding domain, further binds to Callithrix jacchus, Saguinus Oedipus or Saimiri sciureus CD3 epsilon. Callithrix jacchus and Saguinus oedipus are both new world primate belonging to the family of Callitrichidae, while Saimiri sciureus is a new world primate belonging to the family of Cebidae. Said binding domains may preferably selected form sequences identified herein as “I2L” (or synonymously “I2L0”). “12M” and “12M2”, more preferably as “12L” or “I2L0”.

It is preferred for the antigen-binding molecule of the present invention that the preferably second and fourth binding domain which binds to an extracellular epitope of the human and/or the Macaca CD3 epsilon chain comprises a VL region comprising CDR-L1, CDR-L2 and CDR-L3 selected from:
(a) VL region comprising CDR-L1, CDR-L2 and CDR-L3 selected from SEQ ID NOs 40 to 42, 48 to 50, 56 to 58, 64 to 66, 72 to 74 439 to 441, preferably 64 to 66
(b) CDR-L1 as depicted in SEQ ID NO: 27 of WO 2008/119567, CDR-L2 as depicted in SEQ ID NO: 28 of WO 2008/119567 and CDR-L3 as depicted in SEQ ID NO: 29 of WO 2008/119567;
(c) CDR-L1 as depicted in SEQ ID NO: 117 of WO 2008/119567, CDR-L2 as depicted in SEQ ID NO: 118 of WO 2008/119567 and CDR-L3 as depicted in SEQ ID NO: 119 of WO 2008/119567;
(d) CDR-L1 as depicted in SEQ ID NO: 153 of WO 2008/119567, CDR-L2 as depicted in SEQ ID NO: 154 of WO 2008/119567 and CDR-L3 as depicted in SEQ ID NO: 155 of WO 2008/119567; and
(e) VL region comprising CDR-L1, CDR-L2 and CDR-L3 of SEQ ID NOs 420 to 422.

In a furthermore preferred embodiment of the antigen-binding molecule of the present invention, the preferably second and fourth binding domain which binds to an extracellular epitope of the human and/or the Macaca CD3 epsilon chain comprises a VH region comprising CDR-H 1. CDR-H2 and CDR-H3 selected from:
(a) VH region comprising CDR-H1, CDR-H2 and CDR-H3 selected from SEQ ID NOs 37 to 39, 45 to 47, 53 to 55, 61 to 63, 69 to 71 and 436 to 438, preferably 61 to 63;
(b) CDR-H1 as depicted in SEQ ID NO: 12 of WO 2008/119567, CDR-H2 as depicted in SEQ ID NO: 13 of WO 2008/119567 and CDR-H3 as depicted in SEQ ID NO: 14 of WO 2008/119567;
(c) CDR-H1 as depicted in SEQ ID NO: 30 of WO 2008/119567, CDR-H2 as depicted in SEQ ID NO: 31 of WO 2008/119567 and CDR-H3 as depicted in SEQ ID NO: 32 of WO 2008/119567;
(d) CDR-H1 as depicted in SEQ ID NO: 48 of WO 2008/119567. CDR-H2 as depicted in SEQ ID NO: 49 of WO 2008/119567 and CDR-H3 as depicted in SEQ ID NO: 50 of WO 2008/119567;
(c) CDR-H1 as depicted in SEQ ID NO: 66 of WO 2008/119567, CDR-H2 as depicted in SEQ ID NO: 67 of WO 2008/119567 and CDR-H3 as depicted in SEQ ID NO: 68 of WO 2008/119567;
(f) CDR-H1 as depicted in SEQ ID NO: 84 of WO 2008/119567, CDR-H2 as depicted in SEQ ID NO: 85 of WO 2008/119567 and CDR-H3 as depicted in SEQ ID NO: 86 of WO 2008/119567;
(g) CDR-H1 as depicted in SEQ ID NO: 102 of WO 2008/119567. CDR-H2 as depicted in SEQ ID NO: 103 of WO 2008/119567 and CDR-H3 as depicted in SEQ ID NO: 104 of WO 2008/119567;
(h) CDR-H1 as depicted in SEQ ID NO: 120 of WO 2008/119567. CDR-H2 as depicted in SEQ ID NO: 121 of WO 2008/119567 and CDR-H3 as depicted in SEQ ID NO: 122 of WO 2008/119567;
(i) CDR-H1 as depicted in SEQ ID NO: 138 of WO 2008/119567, CDR-H2 as depicted in SEQ ID NO: 139 of WO 2008/119567 and CDR-H3 as depicted in SEQ ID NO: 140 of WO 2008/119567;
(j) CDR-H1 as depicted in SEQ ID NO: 156 of WO 2008/119567, CDR-H2 as depicted in SEQ ID NO: 157 of WO 2008/119567 and CDR-H3 as depicted in SEQ ID NO: 158 of WO 2008/119567;
(k) CDR-H1 as depicted in SEQ ID NO: 174 of WO 2008/119567, CDR-H2 as depicted in SEQ ID NO: 175 of WO 2008/119567 and CDR-H3 as depicted in SEQ ID NO: 176 of WO 2008/119567; and
(l) VH region comprising CDR-H 1, CDR-H2 and CDR-H3 of SEQ ID NOs 423 to 425.

In a preferred embodiment of the antigen-binding molecule of the invention the above described three groups of VL CDRs are combined with the above described ten groups of VH CDRs within the third binding domain to form (30) groups, each comprising CDR-L 1-3 and CDR-H 1-3.

It is preferred for the antigen-binding molecule of the present invention that the third domain which binds to CD3 comprises a VL region selected from the group consisting of those depicted in SEQ ID NOs: 17, 21, 35, 39, 53, 57, 71, 75, 89, 93, 107, 111, 125, 129, 143, 147, 161, 165, 179 or 183 of WO 2008/119567 or, preferably, as depicted in SEQ ID NO: 44, 52, 60, 68 and 76, preferably 68 according to the present invention.

It is also preferred that the third domain which binds to CD3 comprises a VH region selected from the group consisting of those depicted in SEQ ID NO: 15, 19, 33, 37, 51, 55, 69, 73, 87, 91, 105, 109, 123, 127, 141, 145, 159, 163, 177 or 181 of WO 2008/119567 or, preferably, as depicted in SEQ ID NO: SEQ ID NOs 43, 51, 59, 67 and 75, preferably 67 according to the present invention.

More preferably, the antigen-binding molecule of the present invention is characterized by a preferably second and fourth domain which binds to CD3 comprising a VL region and a VH region selected from the group consisting of:
(a) a VL region selected from SEQ ID NOs 44, 52, 60, 68, 76 and 443, and a VH region selected from SEQ ID NOs 43, 51, 59, 67, 75 and 442;
(b) a VL region as depicted in SEQ ID NO: 17 or 21 of WO 2008/119567 and a VH region as depicted in SEQ ID NO: 15 or 19 of WO 2008/119567;
(c) a VL region as depicted in SEQ ID NO: 35 or 39 of WO 2008/119567 and a VH region as depicted in SEQ ID NO: 33 or 37 of WO 2008/119567;
(d) a VL region as depicted in SEQ ID NO: 53 or 57 of WO 2008/119567 and a VH region as depicted in SEQ ID NO: 51 or 55 of WO 2008/119567;
(e) a VL region as depicted in SEQ ID NO: 71 or 75 of WO 2008/119567 and a VH region as depicted in SEQ ID NO: 69 or 73 of WO 2008/119567;
(f) a VL region as depicted in SEQ ID NO: 89 or 93 of WO 2008/119567 and a VH region as depicted in SEQ ID NO: 87 or 91 of WO 2008/119567;
(g) a VL region as depicted in SEQ ID NO: 107 or 111 of WO 2008/119567 and a VH region as depicted in SEQ ID NO: 105 or 109 of WO 2008/119567;
(h) a VL region as depicted in SEQ ID NO: 125 or 129 of WO 2008/119567 and a VH region as depicted in SEQ ID NO: 123 or 127 of WO 2008/119567;
(i) a VL region as depicted in SEQ ID NO: 143 or 147 of WO 2008/119567 and a VH region as depicted in SEQ ID NO: 141 or 145 of WO 2008/119567;
(j) a VL region as depicted in SEQ ID NO: 161 or 165 of WO 2008/119567 and a VH region as depicted in SEQ ID NO: 159 or 163 of WO 2008/119567; and
(k) a VL region as depicted in SEQ ID NO: 179 or 183 of WO 2008/119567 and a VH region as depicted in SEQ ID NO: 177 or 181 of WO 2008/119567.

Also preferred in connection with the antigen-binding molecule of the present invention is a second and forth domain which binds to CD3 comprising a VL region as depicted in SEQ ID NO: 68 and a VH region as depicted in SEQ ID NO: 67.

According to a preferred embodiment of the antigen-binding molecule of the present invention, the first and/or the third domain have the following format: The pairs of VH regions and VL regions are in the format of a single chain antibody (scFv). The VH and VL regions are arranged in the order VH-VL or VL-VH. It is preferred that the VH-region is positioned N-terminally of a linker sequence, and the VL-region is positioned C-terminally of the linker sequence.

The invention further provides an antigen-binding molecule comprising or having an amino acid sequence (full bispecific antigen-binding molecule) selected from the group consisting of any of 673, 676, 679, 682, 685, 688, 691, 694, 697, 700, 703, 706, 709, 712, 715, 718, 721, 724, 727, 730, 733, 736, 739, 742, 745, 748, 751, 754, 757, 760, 763, 766, 769, 772, 775, 778, 781, 784, 787, 790, 793, 796, 799, 802, 805, 808, 811, 814, 817, 820, 823, 826, 829, 832, 835, 838, 841, 844, 847, 850, 853, 856, 859, 862, 865, 868, 871, 1437, 1440, 1443, 1446, 1449, 1452, 1455, 1458, 1461, 1464, 1467, 1470, 1473, 1476, 1479, 1482, 1485, 1488, 1499, 1667, 1670, 1673, 1676, 1679, 1682, 1685, 1688, 1691, 1694, 1697, 1700, 1703, 1706, 1709, 1712, 1715, 1718, 1721, 1724, 1727, 1730, 1733, 1736, 1739, 1742, 1745, 1748, 1751, 1754, 1757, 1760, 1763, 1766, 1769, 1772, 1775, 1778, 1781, 1784, 1787, 1790, 1793, 1796, 1799, 1802, 1805, 1808, 1811, 1814, 1817, 1820, 1823, 1826, and 1829, preferably 1437, or having an amino acid sequence having at least 90, 91, 92, 93, 94 95, 96, 97, 98 or 99% identity to said sequences.

Covalent modifications of the antigen-binding molecules are also included within the scope of this invention, and are generally, but not always, done post-translationally. For example, several types of covalent modifications of the antigen-binding molecule are introduced into the molecule by reacting specific amino acid residues of the antigen-binding molecule with an organic derivatizing agent that is capable of reacting with selected side chains or the N- or C-terminal residues.

Cysteinyl residues most commonly are reacted with α-haloacetates (and corresponding amines), such as chloroacetic acid or chloroacetamide, to give carboxymethyl or carboxyamidomethyl derivatives. Cysteinyl residues also are derivatized by reaction with bromotrifluoroacetone, α-bromo-β-(5-imidozoyl)propionic acid, chloroacetyl phosphate, N-alkylmaleimides, 3-nitro-2-pyridyl disulfide, methyl 2-pyridyl disulfide, p-chloromercuribenzoate, 2-chloromercuri-4-nitrophenol, or chloro-7-nitrobenzo-2-oxa-1,3-diazole.

Histidyl residues are derivatized by reaction with diethylpyrocarbonate at pH 5.5-7.0 because this agent is relatively specific for the histidyl side chain. Para-bromophenacyl bromide also is useful; the reaction is preferably performed in 0.1 M sodium cacodylate at pH 6.0. Lysinyl and amino terminal residues are reacted with succinic or other carboxylic acid anhydrides. Derivatization with these agents has the effect of reversing the charge of the lysinyl residues. Other suitable reagents for derivatizing alpha-amino-containing residues include imidoesters such as methyl picolinimidate; pyridoxal phosphate; pyridoxal; chloroborohydride; trinitrobenzenesulfonic acid; O-methylisourea; 2,4-pentanedione; and transaminase-catalyzed reaction with glyoxylate.

Arginyl residues are modified by reaction with one or several conventional reagents, among them phenylglyoxal, 2,3-butanedione, 1,2-cyclohexanedione, and ninhydrin. Derivatization of arginine residues requires that the reaction be performed in alkaline conditions because of the high pKa of the guanidine functional group. Furthermore, these reagents may react with the groups of lysine as well as the arginine epsilon-amino group.

The specific modification of tyrosyl residues may be made, with particular interest in introducing spectral labels into tyrosyl residues by reaction with aromatic diazonium compounds or tetranitromethane. Most commonly, N-acetylimidizole and tetranitromethane are used to form O-acetyl tyrosyl species and 3-nitro derivatives, respectively. Tyrosyl residues are iodinated using ¹²⁵I or ¹³¹I to prepare labeled proteins for use in radioimmunoassay, the chloramine T method described above being suitable.

Carboxyl side groups (aspartyl or glutamyl) are selectively modified by reaction with carbodiimides (R′—N═C═N—R′), where R and R′ are optionally different alkyl groups, such as 1-cyclohexyl-3-(2-morpholinyl-4-ethyl) carbodiimide or 1-ethyl-3-(4-azonia-4,4-dimethylpentyl) carbodiimide. Furthermore, aspartyl and glutamyl residues are converted to asparaginyl and glutaminyl residues by reaction with ammonium ions.

Derivatization with bifunctional agents is useful for crosslinking the antigen-binding molecules of the present invention to a water-insoluble support matrix or surface for use in a variety of methods. Commonly used crosslinking agents include, e.g., 1,1-bis(diazoacetyl)-2-phenylethane, glutaraldehyde, N-hydroxysuccinimide esters, for example, esters with 4-azidosalicylic acid, homobifunctional imidoesters, including disuccinimidyl esters such as 3,3′-dithiobis(succinimidylpropionate), and bifunctional maleimides such as bis-N-maleimido-1,8-octane. Derivatizing agents such as methyl-3-[(p-azidophenyl)dithio]propioimidate yield photoactivatable intermediates that are capable of forming crosslinks in the presence of light. Alternatively, reactive water-insoluble matrices such as cyanogen bromide-activated carbohydrates and the reactive substrates as described in U.S. Pat. Nos. 3,969,287; 3,691,016; 4,195,128; 4,247,642; 4,229,537; and 4,330,440 are employed for protein immobilization.

Glutaminyl and asparaginyl residues are frequently deamidated to the corresponding glutamyl and aspartyl residues, respectively. Alternatively, these residues are deamidated under mildly acidic conditions. Either form of these residues falls within the scope of this invention.

Other modifications include hydroxylation of proline and lysine, phosphorylation of hydroxyl groups of seryl or threonyl residues, methylation of the α-amino groups of lysine, arginine, and histidine side chains (T. E. Creighton, Proteins: Structure and Molecular Properties, W. H. Freeman & Co., San Francisco, 1983, pp. 79-86), acetylation of the N-terminal amine, and amidation of any C-terminal carboxyl group.

Another type of covalent modification of the antigen-binding molecules included within the scope of this invention comprises altering the glycosylation pattern of the protein. As is known in the art, glycosylation patterns can depend on both the sequence of the protein (e.g., the presence or absence of particular glycosylation amino acid residues, discussed below), or the host cell or organism in which the protein is produced. Particular expression systems are discussed below.

Glycosylation of polypeptides is typically either N-linked or O-linked. N-linked refers to the attachment of the carbohydrate moiety to the side chain of an asparagine residue. The tri-peptide sequences asparagine-X-serine and asparagine-X-threonine, where X is any amino acid except proline, are the recognition sequences for enzymatic attachment of the carbohydrate moiety to the asparagine side chain. Thus, the presence of either of these tri-peptide sequences in a polypeptide creates a potential glycosylation site. O-linked glycosylation refers to the attachment of one of the sugars N-acetylgalactosamine, galactose, or xylose, to a hydroxyamino acid, most commonly serine or threonine, although 5-hydroxyproline or 5-hydroxylysine may also be used.

Addition of glycosylation sites to the antigen-binding molecule is conveniently accomplished by altering the amino acid sequence such that it contains one or more of the above-described tri-peptide sequences (for N-linked glycosylation sites). The alteration may also be made by the addition of, or substitution by, one or more serine or threonine residues to the starting sequence (for O-linked glycosylation sites). For case, the amino acid sequence of an antigen-binding molecule is preferably altered through changes at the DNA level, particularly by mutating the DNA encoding the polypeptide at preselected bases such that codons are generated that will translate into the desired amino acids.

Another means of increasing the number of carbohydrate moieties on the antigen-binding molecule is by chemical or enzymatic coupling of glycosides to the protein. These procedures are advantageous in that they do not require production of the protein in a host cell that has glycosylation capabilities for N- and O-linked glycosylation. Depending on the coupling mode used, the sugar(s) may be attached to (a) arginine and histidine, (b) free carboxyl groups, (c) free sulfhydryl groups such as those of cysteine. (d) free hydroxyl groups such as those of serine, threonine, or hydroxyproline, (e) aromatic residues such as those of phenylalanine, tyrosine, or tryptophan, or (f) the amide group of glutamine. These methods are described in WO 87/05330, and in Aplin and Wriston, 1981, CRC Crit. Rev. Biochem., pp. 259-306.

Removal of carbohydrate moieties present on the starting antigen-binding molecule may be accomplished chemically or enzymatically. Chemical deglycosylation requires exposure of the protein to the compound trifluoromethanesulfonic acid, or an equivalent compound. This treatment results in the cleavage of most or all sugars except the linking sugar (N-acetylglucosamine or N-acetylgalactosamine), while leaving the polypeptide intact. Chemical deglycosylation is described by Hakimuddin et al., 1987, Arch. Biochem. Biophys. 259:52 and by Edge et al., 1981, Anal. Biochem. 118:131. Enzymatic cleavage of carbohydrate moieties on polypeptides can be achieved by the use of a variety of endo- and exo-glycosidases as described by Thotakura et al., 1987, Meth. Enzymol. 138:350. Glycosylation at potential glycosylation sites may be prevented by the use of the compound tunicamycin as described by Duskin et al., 1982, J. Biol. Chem. 257:3105. Tunicamycin blocks the formation of protein-N-glycoside linkages.

Other modifications of the antigen-binding molecule are also contemplated herein. For example, another type of covalent modification of the antigen-binding molecule comprises linking the antigen-binding molecule to various non-proteinaceous polymers, including, but not limited to, various polyols such as polyethylene glycol, polypropylene glycol, polyoxyalkylenes, or copolymers of polyethylene glycol and polypropylene glycol, in the manner set forth in U.S. Pat. Nos. 4,640,835; 4,496,689; 4,301,144; 4,670,417; 4,791,192 or 4,179,337. In addition, as is known in the art, amino acid substitutions may be made in various positions within the antigen-binding molecule, e.g. in order to facilitate the addition of polymers such as PEG.

In some embodiments, the covalent modification of the antigen-binding molecules of the invention comprises the addition of one or more labels. The labelling group may be coupled to the antigen-binding molecule via spacer arms of various lengths to reduce potential steric hindrance. Various methods for labelling proteins are known in the art and can be used in performing the present invention. The term “label” or “labelling group” refers to any detectable label. In general, labels fall into a variety of classes, depending on the assay in which they are to be detected—the following examples include, but are not limited to:
a) isotopic labels, which may be radioactive or heavy isotopes, such as radioisotopes or radionuclides (e.g., ³H, ¹⁴C, ¹⁵N, ³⁵S, ⁸⁹Zr, ⁹⁰Y, ⁹⁹Tc, ¹¹¹In, ¹²⁵I, ¹³¹I)
b) magnetic labels (e.g., magnetic particles)
c) redox active moieties
d) optical dyes (including, but not limited to, chromophores, phosphors and fluorophores) such as fluorescent groups (e.g., FITC, rhodamine, lanthanide phosphors), chemiluminescent groups, and fluorophores which can be either “small molecule” fluors or proteinaceous fluors
e) enzymatic groups (e.g. horseradish peroxidase, β-galactosidase, luciferase, alkaline phosphatase)
f) biotinylated groups
g) predetermined polypeptide epitopes recognized by a secondary reporter (e.g., leucine zipper pair sequences, binding sides for secondary antibodies, metal binding domains, epitope tags, etc.)

By “fluorescent label” is meant any molecule that may be detected via its inherent fluorescent properties. Suitable fluorescent labels include, but are not limited to, fluorescein, rhodamine, tetramethylrhodamine, cosin, erythrosin, coumarin, methyl-coumarins, pyrene, Malacite green, stilbene, Lucifer Yellow, Cascade BlueJ, Texas Red, IAEDANS, EDANS, BODIPY FL, LC Red 640, Cy 5, Cy 5.5. LC Red 705, Oregon green, the Alexa-Fluor dyes (Alexa Fluor 350, Alexa Fluor 430, Alexa Fluor 488, Alexa Fluor 546, Alexa Fluor 568, Alexa Fluor 594, Alexa Fluor 633, Alexa Fluor 660, Alexa Fluor 680), Cascade Blue, Cascade Yellow and R-phycoerythrin (PE) (Molecular Probes, Eugene, OR), FITC, Rhodamine, and Texas Red (Pierce, Rockford, IL), Cy5, Cy5.5, Cy7 (Amersham Life Science, Pittsburgh, PA). Suitable optical dyes, including fluorophores, are described in Molecular Probes Handbook by Richard P. Haugland.

Suitable proteinaceous fluorescent labels also include, but are not limited to, green fluorescent protein, including a Renilla, Ptilosarcus, or Aequorea species of GFP (Chalfie et al., 1994, Science 263:802-805), EGFP (Clontech Laboratories, Inc., Genbank Accession Number U55762), blue fluorescent protein (BFP, Quantum Biotechnologies, Inc. 1801 de Maisonneuve Blvd. West, 8th Floor, Montreal, Quebec, Canada H3H 1J9; Stauber, 1998, Biotechniques 24:462-471; Heim et al., 1996, Curr. Biol. 6:178-182), enhanced yellow fluorescent protein (EYFP, Clontech Laboratories, Inc.), luciferase (Ichiki et al., 1993, J. Immunol. 150:5408-5417), β galactosidase (Nolan et al., 1988, Proc. Natl. Acad. Sci. U.S.A. 85:2603-2607) and Renilla (WO92/15673, WO95/07463, WO98/14605, WO98/26277, WO99/49019, U.S. Pat. Nos. 5,292,658; 5,418,155; 5,683,888; 5,741,668; 5,777,079; 5,804.387; 5,874.304; 5,876,995; 5,925,558).

The antigen-binding molecule of the invention may also comprise additional domains, which are e.g. helpful in the isolation of the molecule or relate to an adapted pharmacokinetic profile of the molecule. Domains helpful for the isolation of an antigen-binding molecule may be selected from peptide motives or secondarily introduced moieties, which can be captured in an isolation method, e.g. an isolation column. Non-limiting embodiments of such additional domains comprise peptide motives known as Myc-tag, HAT-tag, HA-tag, TAP-tag, GST-tag, chitin binding domain (CBD-tag), maltose binding protein (MBP-tag), Flag-tag, Strep-tag and variants thereof (e.g. StrepII-tag) and His-tag. All herein disclosed antigen-binding molecules may comprise a His-tag domain, which is generally known as a repeat of consecutive His residues in the amino acid sequence of a molecule, preferably of five, and more preferably of six His residues (hexa-histidine). The His-tag may be located e.g. at the N- or C-terminus of the antigen-binding molecule, preferably it is located at the C-terminus. Most preferably, a hexa-histidine tag (HHHHHH) (SEQ ID NO:16) is linked via peptide bond to the C-terminus of the antigen-binding molecule according to the invention. Additionally, a conjugate system of PLGA-PEG-PLGA may be combined with a poly-histidine tag for sustained release application and improved pharmacokinetic profile.

Amino acid sequence modifications of the antigen-binding molecules described herein are also contemplated. For example, it may be desirable to improve the binding affinity and/or other biological properties of the antigen-binding molecule. Amino acid sequence variants of the antigen-binding molecules are prepared by introducing appropriate nucleotide changes into the antigen-binding molecules nucleic acid, or by peptide synthesis. All of the below described amino acid sequence modifications should result in an antigen-binding molecule which still retains the desired biological activity (binding to CS1, BCMA, CD20, CD22, FLT3, CD123, CLL1, CHD3, MSLN, or EpCAM and to CD3) of the unmodified parental molecule.

The term “amino acid” or “amino acid residue” typically refers to an amino acid having its art recognized definition such as an amino acid selected from the group consisting of: alanine (Ala or A); arginine (Arg or R); asparagine (Asn or N); aspartic acid (Asp or D); cysteine (Cys or C); glutamine (GIn or Q); glutamic acid (Glu or E); glycine (Gly or G); histidine (His or H); isoleucine (He or I); leucine (Leu or L); lysine (Lys or K); methionine (Met or M); phenylalanine (Phe or F); pro line (Pro or P); serine (Ser or S); threonine (Thr or T); tryptophan (Trp or W); tyrosine (Tyr or Y); and valine (Val or V), although modified, synthetic, or rare amino acids may be used as desired. Generally, amino acids can be grouped as having a nonpolar side chain (e.g., Ala, Cys, He, Leu, Met, Phe, Pro, Val); a negatively charged side chain (e.g., Asp, Glu); a positively charged sidechain (e.g., Arg, His, Lys); or an uncharged polar side chain (e.g., Asn, Cys, Gln, Gly, His, Met, Phe, Ser, Thr, Trp, and Tyr).

Amino acid modifications include, for example, deletions from, and/or insertions into, and/or substitutions of, residues within the amino acid sequences of the antigen-binding molecules. Any combination of deletion, insertion, and substitution is made to arrive at the final construct, provided that the final construct possesses the desired characteristics. The amino acid changes also may alter post-translational processes of the antigen-binding molecules, such as changing the number or position of glycosylation sites.

For example, 1, 2, 3, 4, 5, or 6 amino acids may be inserted, substituted or deleted in each of the CDRs (of course, dependent on their length), while 1, 2, 3, 4, 5, 6, 7, 8, 9, 10, 11, 12, 13, 14, 15, 16, 17, 18, 19, 20, or 25 amino acids may be inserted, substituted or deleted in each of the FRs. Preferably, amino acid sequence insertions into the antigen-binding molecule include amino- and/or carboxyl-terminal fusions ranging in length from 1, 2, 3, 4, 5, 6, 7, 8, 9 or 10 residues to polypeptides containing a hundred or more residues, as well as intra-sequence insertions of single or multiple amino acid residues. An insertional variant of the antigen-binding molecule of the invention includes the fusion to the N-terminus or to the C-terminus of the antigen-binding molecule of an enzyme or the fusion to a polypeptide.

The sites of greatest interest for substitutional mutagenesis include (but are not limited to) the CDRs of the heavy and/or light chain, in particular the hypervariable regions, but FR alterations in the heavy and/or light chain are also contemplated. The substitutions are preferably conservative substitutions as described herein. Preferably, 1, 2, 3, 4, 5, 6, 7, 8, 9, or 10 amino acids may be substituted in a CDR, while 1, 2, 3, 4, 5, 6, 7, 8, 9, 10, 11, 12, 13, 14, 15, 16, 17, 18, 19, 20, or 25 amino acids may be substituted in the framework regions (FRs), depending on the length of the CDR or FR. For example, if a CDR sequence encompasses 6 amino acids, it is envisaged that one, two or three of these amino acids are substituted. Similarly, if a CDR sequence encompasses 15 amino acids it is envisaged that one, two, three, four, five or six of these amino acids are substituted.

A useful method for identification of certain residues or regions of the antigen-binding molecules that are preferred locations for mutagenesis is called “alanine scanning mutagenesis” as described by Cunningham and Wells in Science, 244: 1081-1085 (1989). Here, a residue or group of target residues within the antigen-binding molecule is/are identified (e.g. charged residues such as arg, asp, his, lys, and glu) and replaced by a neutral or negatively charged amino acid (most preferably alanine or polyalanine) to affect the interaction of the amino acids with the epitope.

Those amino acid locations demonstrating functional sensitivity to the substitutions are then refined by introducing further or other variants at, or for, the sites of substitution. Thus, while the site or region for introducing an amino acid sequence variation is predetermined, the nature of the mutation per se needs not to be predetermined. For example, to analyze or optimize the performance of a mutation at a given site, alanine scanning or random mutagenesis may be conducted at a target codon or region, and the expressed antigen-binding molecule variants are screened for the optimal combination of desired activity. Techniques for making substitution mutations at predetermined sites in the DNA having a known sequence are well known, for example, M13 primer mutagenesis and PCR mutagenesis. Screening of the mutants is done using assays of antigen binding activities, such as CS1, BCMA, CD20, CD22, FLT3, CD123, CLL1, CHD3, MSLN, or EpCAM or CD3 binding.

Generally, if amino acids are substituted in one or more or all of the CDRs of the heavy and/or light chain, it is preferred that the then-obtained “substituted” sequence is at least 60% or 65%, more preferably 70% or 75%, even more preferably 80% or 85%, and particularly preferably 90% or 95% identical to the “original” CDR sequence. This means that it is dependent of the length of the CDR to which degree it is identical to the “substituted” sequence. For example, a CDR having 5 amino acids is preferably 80% identical to its substituted sequence in order to have at least one amino acid substituted. Accordingly, the CDRs of the antigen-binding molecule may have different degrees of identity to their substituted sequences, e.g., CDRL1 may have 80%, while CDRL3 may have 90%.

Preferred substitutions (or replacements) are conservative substitutions. However, any substitution (including non-conservative substitution or one or more from the “exemplary substitutions” listed in Table 3, below) is envisaged as long as the antigen-binding molecule retains its capability to bind to CS1, BCMA, CD20, CD22, FLT3, CD123, CLL1, CHD3, MSLN, or EpCAM via the first domain and to CD3 epsilon via the second domain and/or its CDRs have an identity to the then substituted sequence (at least 60% or 65%, more preferably 70% or 75%, even more preferably 80% or 85%, and particularly preferably 90% or 95% identical to the “original” CDR sequence).

Conservative substitutions are shown in Table 3 under the heading of “preferred substitutions”. If such substitutions result in a change in biological activity, then more substantial changes, denominated “exemplary substitutions” in Table 3, or as further described below in reference to amino acid classes, may be introduced and the products screened for a desired characteristic.

TABLE 3
Amino acid substitutions

| | | Preferred |
|---|---|---|
| Original | Exemplary Substitutions | Substitutions |
| Ala (A) | val, leu, ile | Val |
| Arg (R) | lys, gln, asn | Lys |
| Asn (N) | gln, his, asp, lys, arg | Gln |
| Asp (D) | glu, asn | Glu |
| Cys (C) | ser, ala | ser |
| Gln (Q) | asn, glu | asn |
| Glu (E) | asp, gln | asp |
| Gly (G) | Ala | ala |
| His (H) | asn, gln, lys, arg | arg |
| Ile (I) | leu, val, met, ala, phe | leu |
| Leu (L) | norleucine, ile, val, met, ala | ile |
| Lys (K) | arg, gln, asn | arg |
| Met (M) | leu, phe, ile | leu |
| Phe (F) | leu, val, ile, ala, tyr | tyr |
| Pro (P) | Ala | ala |
| Ser (S) | Thr | thr |
| Thr (T) | Ser | ser |
| Trp (W) | tyr, phe | tyr |
| Tyr (Y) | trp, phe, thr, ser | phe |
| Val (V) | ile, leu, met, phe, ala | leu |

Substantial modifications in the biological properties of the antigen-binding molecule of the present invention are accomplished by selecting substitutions that differ significantly in their effect on maintaining (a) the structure of the polypeptide backbone in the area of the substitution, for example, as a sheet or helical conformation, (b) the charge or hydrophobicity of the molecule at the target site, or (c) the bulk of the side chain. Naturally occurring residues are divided into groups based on common side-chain properties: (1) hydrophobic: norleucine, met, ala, val, leu, ile; (2) neutral hydrophilic: cys, ser, thr; asn, gln (3) acidic: asp, glu; (4) basic: his, lys, arg; (5) residues that influence chain orientation: gly, pro; and (6) aromatic: trp, tyr, phe.

Non-conservative substitutions will entail exchanging a member of one of these classes for another class. Any cysteine residue not involved in maintaining the proper conformation of the antigen-binding molecule may be substituted, generally with serine, to improve the oxidative stability of the molecule and prevent aberrant crosslinking. Conversely, cysteine bond(s) may be added to the antibody to improve its stability (particularly where the antibody is an antibody fragment such as an Fv fragment).

For amino acid sequences, sequence identity and/or similarity is determined by using standard techniques known in the art, including, but not limited to, the local sequence identity algorithm of Smith and Waterman, 1981, Adv. Appl. Math. 2:482, the sequence identity alignment algorithm of Needleman and Wunsch, 1970, J. Mol. Biol. 48:443, the search for similarity method of Pearson and Lipman, 1988, Proc. Nat. Acad. Sci. U.S.A. 85:2444, computerized implementations of these algorithms (GAP, BESTFIT, FASTA, and TFASTA in the Wisconsin Genetics Software Package, Genetics Computer Group, 575 Science Drive, Madison, Wis.), the Best Fit sequence program described by Devereux et al., 1984, Nucl. Acid Res. 12:387-395, preferably using the default settings, or by inspection. Preferably, percent identity is calculated by FastDB based upon the following parameters: mismatch penalty of 1; gap penalty of 1; gap size penalty of 0.33; and joining penalty of 30, “Current Methods in Sequence Comparison and Analysis,” Macromolecule Sequencing and Synthesis, Selected Methods and Applications, pp 127-149 (1988), Alan R. Liss, Inc.

An example of a useful algorithm is PILEUP. PILEUP creates a multiple sequence alignment from a group of related sequences using progressive, pairwise alignments. It can also plot a tree showing the clustering relationships used to create the alignment. PILEUP uses a simplification of the progressive alignment method of Feng & Doolittle, 1987, J. Mol. Evol. 35:351-360; the method is similar to that described by Higgins and Sharp, 1989, CABIOS 5:151-153. Useful PILEUP parameters including a default gap weight of 3.00, a default gap length weight of 0.10, and weighted end gaps.

Another example of a useful algorithm is the BLAST algorithm, described in: Altschul et al., 1990, J. Mol. Biol. 215:403-410; Altschul et al., 1997, Nucleic Acids Res. 25:3389-3402; and Karin et al., 1993, Proc. Natl. Acad. Sci. U.S.A. 90:5873-5787. A particularly useful BLAST program is the WU-BLAST-2 program which was obtained from Altschul et al., 1996, Methods in Enzymology 266:460-480. WU-BLAST-2 uses several search parameters, most of which are set to the default values. The adjustable parameters are set with the following values: overlap span=1, overlap fraction=0.125, word threshold (T)=II. The HSP S and HSP S2 parameters are dynamic values and are established by the program itself depending upon the composition of the particular sequence and composition of the particular database against which the sequence of interest is being searched; however, the values may be adjusted to increase sensitivity.

An additional useful algorithm is gapped BLAST as reported by Altschul et al., 1993, Nucl. Acids Res. 25:3389-3402. Gapped BLAST uses BLOSUM-62 substitution scores; threshold T parameter set to 9; the two-hit method to trigger ungapped extensions, charges gap lengths of k a cost of 10+k; Xu set to 16, and Xg set to 40 for database search stage and to 67 for the output stage of the algorithms. Gapped alignments are triggered by a score corresponding to about 22 bits.

Generally, the amino acid homology, similarity, or identity between individual variant CDRs or VH/VL sequences are at least 60% to the sequences depicted herein, and more typically with preferably increasing homologies or identities of at least 65% or 70%, more preferably at least 75% or 80%, even more preferably at least 85%, 90%, 91%, 92%, 93%, 94%, 95%, 96%, 97%, 98%, 99%, and almost 100%. In a similar manner, “percent (%) nucleic acid sequence identity” with respect to the nucleic acid sequence of the binding proteins identified herein is defined as the percentage of nucleotide residues in a candidate sequence that are identical with the nucleotide residues in the coding sequence of the antigen-binding molecule. A specific method utilizes the BLASTN module of WU-BLAST-2 set to the default parameters, with overlap span and overlap fraction set to 1 and 0.125, respectively.

Generally, the nucleic acid sequence homology, similarity, or identity between the nucleotide sequences encoding individual variant CDRs or VH/VL sequences and the nucleotide sequences depicted herein are at least 60%, and more typically with preferably increasing homologies or identities of at least 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99%, and almost 100%. Thus, a “variant CDR” or a “variant VH/VL region” is one with the specified homology, similarity, or identity to the parent CDR/VH/VL of the invention, and shares biological function, including, but not limited to, at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% of the specificity and/or activity of the parent CDR or VH/VL.

In one embodiment, the percentage of identity to human germline of the antigen-binding molecules according to the invention is ≥70% or ≥75%, more preferably ≥80% or ≥85%, even more preferably >90%, and most preferably ≥91%, ≥92%, ≥93%, ≥94%, ≥95% or even ≥96%. Identity to human antibody germline gene products is thought to be an important feature to reduce the risk of therapeutic proteins to elicit an immune response against the drug in the patient during treatment. Hwang & Foote (“Immunogenicity of engineered antibodies”; Methods 36 (2005) 3-10) demonstrate that the reduction of non-human portions of drug antigen-binding molecules leads to a decrease of risk to induce anti-drug antibodies in the patients during treatment. By comparing an exhaustive number of clinically evaluated antibody drugs and the respective immunogenicity data, the trend is shown that humanization of the V-regions of antibodies makes the protein less immunogenic (average 5.1% of patients) than antibodies carrying unaltered non-human V regions (average 23.59% of patients). A higher degree of identity to human sequences is hence desirable for V-region based protein therapeutics in the form of antigen-binding molecules. For this purpose of determining the germline identity, the V-regions of VL can be aligned with the amino acid sequences of human germline V segments and J segments (http://vbase.mrc-cpe.cam.ac.uk/) using Vector NTI software and the amino acid sequence calculated by dividing the identical amino acid residues by the total number of amino acid residues of the VL in percent. The same can be for the VH segments (http://vbase.mrc-cpe.cam.ac.uk/) with the exception that the VH CDR3 may be excluded due to its high diversity and a lack of existing human germline VH CDR3 alignment partners. Recombinant techniques can then be used to increase sequence identity to human antibody germline genes.

In a further embodiment, the bispecific antigen-binding molecules of the present invention exhibit high monomer yields under standard research scale conditions, e.g., in a standard two-step purification process. Preferably the monomer yield of the antigen-binding molecules according to the invention is >0.25 mg/L supernatant, more preferably ≥0.5 mg/L, even more preferably ≥1 mg/L, and most preferably ≥3 mg/L supernatant.

Likewise, the yield of the dimeric antigen-binding molecule isoforms and hence the monomer percentage (i.e., monomer: (monomer+dimer)) of the antigen-binding molecules can be determined. The productivity of monomeric and dimeric antigen-binding molecules and the calculated monomer percentage can e.g. be obtained in the SEC purification step of culture supernatant from standardized research-scale production in roller bottles. In one embodiment, the monomer percentage of the antigen-binding molecules is ≥80%, more preferably ≥85%, even more preferably ≥90%, and most preferably ≥95%.

In one embodiment, the antigen-binding molecules have a preferred plasma stability (ratio of EC50 with plasma to EC50 w/o plasma) of ≤5 or ≤4, more preferably ≤3.5 or ≤ 3, even more preferably ≤2.5 or ≤2, and most preferably ≤1.5 or ≤1. The plasma stability of an antigen-binding molecule can be tested by incubation of the construct in human plasma at 37° C. for 24 hours followed by EC50 determination in a ⁵¹chromium release cytotoxicity assay. The effector cells in the cytotoxicity assay can be stimulated enriched human CD8 positive T cells. Target cells can e.g. be CHO cells transfected with human CS1, BCMA, CD20, CD22, FLT3, CD123, CLL1, CHD3, MSLN, or EpCAM. The effector to target cell (E:T) ratio can be chosen as 10:1 or 5:1. The human plasma pool used for this purpose is derived from the blood of healthy donors collected by EDTA coated syringes. Cellular components are removed by centrifugation and the upper plasma phase is collected and subsequently pooled. As control, antigen-binding molecules are diluted immediately prior to the cytotoxicity assay in RPMI-1640 medium. The plasma stability is calculated as ratio of EC50 (after plasma incubation) to EC50 (control).

It is furthermore preferred that the monomer to dimer conversion of antigen-binding molecules of the invention is low. The conversion can be measured under different conditions and analyzed by high performance size exclusion chromatography. For example, incubation of the monomeric isoforms of the antigen-binding molecules can be carried out for 7 days at 37° C. and concentrations of e.g. 100 μg/ml or 250 μg/ml in an incubator. Under these conditions, it is preferred that the antigen-binding molecules of the invention show a dimer percentage that is <5%, more preferably <4%, even more preferably ≤3%, even more preferably <2.5%, even more preferably ≤2%, even more preferably ≤1.5%, and most preferably ≤1% or ≤0.5% or even 0%.

It is also preferred that the bispecific antigen-binding molecules of the present invention present with very low dimer conversion after a number of freeze/thaw cycles. For example, the antigen-binding molecule monomer is adjusted to a concentration of 250 μg/ml e.g. in generic formulation buffer and subjected to three freeze/thaw cycles (freezing at −80° C. for 30 min followed by thawing for 30 min at room temperature), followed by high performance SEC to determine the percentage of initially monomeric antigen-binding molecule, which had been converted into dimeric antigen-binding molecule. Preferably the dimer percentages of the bispecific antigen-binding molecules are ≤5%, more preferably <4%, even more preferably ≤3%, even more preferably ≤2.5%, even more preferably ≤2%, even more preferably ≤1.5%, and most preferably ≤1% or even ≤0.5%, for example after three freeze/thaw cycles.

The bispecific antigen-binding molecules of the present invention preferably show a favorable thermostability with aggregation temperatures ≥45° C. or ≥50° C., more preferably ≥52° C. or ≥54° C., even more preferably ≥56° C. or ≥57° C., and most preferably ≥58° C. or ≥59° C. The thermostability parameter can be determined in terms of antibody aggregation temperature as follows: Antibody solution at a concentration 250 μg/ml is transferred into a single use cuvette and placed in a Dynamic Light Scattering (DLS) device. The sample is heated from 40° C. to 70° ° C. at a heating rate of 0.5° C./min with constant acquisition of the measured radius. Increase of radius indicating melting of the protein and aggregation is used to calculate the aggregation temperature of the antibody.

Alternatively, temperature melting curves can be determined by Differential Scanning calorimetry (DSC) to determine intrinsic biophysical protein stabilities of the antigen-binding molecules. These experiments are performed using a MicroCal LLC (Northampton, MA, U.S.A) VP-DSC device. The energy uptake of a sample containing an antigen-binding molecule is recorded from 20° ° C. to 90° C. compared to a sample containing only the formulation buffer. The antigen-binding molecules are adjusted to a final concentration of 250 μg/ml e.g. in SEC running buffer. For recording of the respective melting curve, the overall sample temperature is increased stepwise. At each temperature T energy uptake of the sample and the formulation buffer reference is recorded. The difference in energy uptake Cp (kcal/mole/° C.) of the sample minus the reference is plotted against the respective temperature. The melting temperature is defined as the temperature at the first maximum of energy uptake.

The CS1, BCMA, CD20, CD22, FLT3, CD123, CLL1, CHD3, MSLN, or EpCAMxCD3 bispecific antigen-binding molecules of the invention are also envisaged to have a turbidity (as measured by OD340 after concentration of purified monomeric antigen-binding molecule to 2.5 mg/ml and overnight incubation) of ≤0.2, preferably of ≤0.15, more preferably of ≤0.12, even more preferably of ≤0.1, and most preferably of ≤0.08.

In a further embodiment the antigen-binding molecule according to the invention is stable at physiologic or slightly lower pH, i.e. about pH 7.4 to 6.0. The more tolerant the antigen-binding molecule behaves at unphysiologic pH such as about pH 6.0, the higher is the recovery of the antigen-binding molecule eluted from an ion exchange column relative to the total amount of loaded protein. Recovery of the antigen-binding molecule from an ion (e.g., cation) exchange column at about pH 6.0 is preferably ≥30%, more preferably ≥40%, more preferably ≥50%, even more preferably ≥60%, even more preferably ≥70%, even more preferably ≥80%, even more preferably ≥90%, even more preferably ≥95%, and most preferably ≥99%.

It is furthermore envisaged that the bispecific antigen-binding molecules of the present invention exhibit therapeutic efficacy or anti-tumor activity. This can e.g. be assessed in a study as disclosed in the following generalized example of an advanced stage human tumor xenograft model:

On day 1 of the study, 5×10⁶ cells of a human target cell antigen (here: CS1, BCMA, CD20, CD22, FLT3, CD123, CLL1, CHD3, MSLN, or EpCAM) positive cancer cell line are subcutaneously injected in the right dorsal flank of female NOD/SCID mice. When the mean tumor volume reaches about 100 mm3, in vitro expanded human CD3 positive T cells are transplanted into the mice by injection of about 2×10⁷ cells into the peritoneal cavity of the animals. Mice of vehicle control group 1 do not receive effector cells and are used as an untransplanted control for comparison with vehicle control group 2 (receiving effector cells) to monitor the impact of T cells alone on tumor growth. The treatment with a bispecific antigen-binding molecule starts when the mean tumor volume reaches about 200 mm³. The mean tumor size of each treatment group on the day of treatment start should not be statistically different from any other group (analysis of variance). Mice are treated with 0.5 mg/kg/day of a CS1, BCMA, CD20, CD22, FLT3, CD123, CLL1, CHD3, MSLN, or EpCAM and CD3 bispecific antigen-binding molecule by intravenous bolus injection for about 15 to 20 days. Tumors are measured by caliper during the study and progress evaluated by intergroup comparison of tumor volumes (TV). The tumor growth inhibition T/C [%] is determined by calculating TV as T/C %=100×(median TV of analyzed group)/(median TV of control group 2).

The skilled person knows how to modify or adapt certain parameters of this study, such as the number of injected tumor cells, the site of injection, the number of transplanted human T cells, the amount of bispecific antigen-binding molecules to be administered, and the timelines, while still arriving at a meaningful and reproducible result. Preferably, the tumor growth inhibition T/C [%] is ≤70 or ≤60, more preferably ≤50 or ≤40, even more preferably ≤30 or ≤20 and most preferably ≤10 or ≤5 or even ≤2.5. Tumor growth inhibition is preferably close to 100%.

In a preferred embodiment of the antigen-binding molecule of the invention the antigen-binding molecule is a single chain antigen-binding molecule.

Also in a preferred embodiment of the antigen-binding molecule of the invention said spacer comprises in an amino to carboxyl order:
hinge-CH2-CH3-linker-hinge-CH2-CH3.

In one embodiment of the invention each of said polypeptide monomers of the spacer has an amino acid sequence that is at least 90% identical to a sequence selected from the group consisting of: SEQ ID NO: 17-24. In a preferred embodiment or the invention each of said polypeptide monomers has an amino acid sequence selected from SEQ ID NO: 17-24.

Also in one embodiment of the invention the CH2 domain of one or preferably each (both) polypeptide monomers of the spacer comprises an intra domain cysteine disulfide bridge. As known in the art the term “cysteine disulfide bridge” refers to a functional group with the general structure R—S—S—R. The linkage is also called an SS-bond or a disulfide bridge and is derived by the coupling of two thiol groups of cysteine residues. It is particularly preferred for the antigen-binding molecule of the invention that the cysteines forming the cysteine disulfide bridge in the mature antigen-binding molecule are introduced into the amino acid sequence of the CH2 domain corresponding to 309 and 321 (Kabat numbering).

In one embodiment of the invention a glycosylation site in Kabat position 314 of the CH2 domain is removed. It is preferred that this removal of the glycosylation site is achieved by a N314X substitution, wherein X is any amino acid excluding Q. Said substitution is preferably a N314G. In a more preferred embodiment, said CH2 domain additionally comprises the following substitutions (position according to Kabat) V321C and R309C (these substitutions introduce the intra domain cysteine disulfide bridge at Kabat positions 309 and 321).

It is assumed that the preferred features of the antigen-binding molecule of the invention compared e.g. to the bispecific heteroFc antigen-binding molecule known in the art may be inter alia related to the introduction of the above described modifications in the CH2 domain. Thus, it is preferred for the construct of the invention that the CH2 domains in the spacer of the antigen-binding molecule of the invention comprise the intra domain cysteine disulfide bridge at Kabat positions 309 and 321 and/or the glycosylation site at Kabat position 314 is removed, preferably by a N314G substitution.

In a further preferred embodiment of the invention the CH2 domains in the spacer of the antigen-binding molecule of the invention comprise the intra domain cysteine disulfide bridge at Kabat positions 309 and 321 and the glycosylation site at Kabat position 314 is removed by a N314G substitution. Most preferably, the polypeptide monomer of the spacer of the antigen-binding molecule of the invention has an amino acid sequence selected from the group consisting of SEQ ID NO: 17 and 18.

In one embodiment the invention provides an antigen-binding molecule, wherein:
(i) the first domain comprises two antibody variable domains and the second domain comprises two antibody variable domains;
(ii) the first domain comprises one antibody variable domain and the second domain comprises two antibody variable domains;
(iii) the first domain comprises two antibody variable domains and the second domain comprises one antibody variable domain; or
(iv) the first domain comprises one antibody variable domain and the second domain comprises one antibody variable domain.

Accordingly, the first and the second domain may be binding domains comprising each two antibody variable domains such as a VH and a VL domain. Examples for such binding domains comprising two antibody variable domains where described herein above and comprise e.g. Fv fragments, scFv fragments or Fab fragments described herein above. Alternatively, either one or both of those binding domains may comprise only a single variable domain. Examples for such single domain binding domains where described herein above and comprise e.g. nanobodies or single variable domain antibodies comprising merely one variable domain, which may be VHH, VH or VL, that specifically bind an antigen or epitope independently of other V regions or domains.

In a preferred embodiment of the antigen-binding molecule of the invention second and third binding domain are fused to the spacer via a peptide linker. Preferred peptide linker have been described herein above and are characterized by the amino acid sequence Gly-Gly-Gly-Gly-Ser, i.e. Gly₄Ser (SEQ ID NO: 7), or polymers thereof, i.e. (Gly₄Ser)x, where x is an integer of 1 or greater (e.g. 2 or 3). A particularly preferred linker for the fusion of the first and second domain to the spacer is depicted in SEQ ID NO: 7.

The antigen-binding molecule of the present invention comprises a first domain which binds to CS1, BCMA, CD20, CD22, FLT3, CD123, CLL1, CHD3, MSLN, or EpCAM, preferably to the extracellular domain(s) (ECD) of CS1, BCMA, CD20, CD22, FLT3, CD123, CLL1, CHD3, MSLN, or EpCAM. It is understood that the term “binding to the extracellular domain of CS1, BCMA, CD20, CD22, FLT3, CD123, CLL1, CHD3, MSLN, or EpCAM”, in the context of the present invention, implies that the binding domain binds to CS1, BCMA, CD20, CD22, FLT3, CD123, CLL1, CHD3, MSLN, or EpCAM expressed on the surface of a target cell. The first domain according to the invention hence preferably binds to CS1, BCMA, CD20, CD22, FLT3, CD123, CLL1, CHD3, MSLN, or EpCAM when it is expressed by naturally expressing cells or cell lines, and/or by cells or cell lines transformed or (stably/transiently) transfected with CS1, BCMA, CD20, CD22, FLT3, CD123, CLL1, CHD3, MSLN, or EpCAM. In a preferred embodiment the first binding domain also binds to CS1, BCMA, CD20, CD22, FLT3, CD123, CLL1, CHD3, MSLN, or EpCAM when CS1, BCMA, CD20, CD22, FLT3, CD123, CLL1, CHD3, MSLN, or EpCAM is used as a “target” or “ligand” molecule in an in vitro binding assay such as BIAcore or Scatchard. The “target cell” can be any prokaryotic or eukaryotic cell expressing CS1, BCMA, CD20, CD22, FLT3, CD123, CLL1, CHD3, MSLN, or EpCAM on its surface; preferably the target cell is a cell that is part of the human or animal body, such as a specific CS1, BCMA, CD20, CD22, FLT3, CD123, CLL1, CHD3, MSLN, or EpCAM expressing cancer or tumor cell.

Preferably, the first binding domain binds to human CS1, BCMA, CD20, CD22, FLT3, CD123, CLL1, CHD3, MSLN, or EpCAM/CS1, BCMA, CD20, CD22, FLT3, CD123, CLL1, CHD3, MSLN, or EpCAM ECD. In a further preferred embodiment, it binds to macaque CS1, BCMA, CD20, CD22, FLT3, CD123, CLL1, CHD3, MSLN, or EpCAM/CS1, BCMA, CD20, CD22, FLT3, CD123, CLL1, CHD3, MSLN, or EpCAM ECD. According to the most preferred embodiment, it binds to both the human and the macaque CS1, BCMA, CD20, CD22, FLT3, CD123, CLL1, CHD3, MSLN, or EpCAM/CS1, BCMA, CD20, CD22, FLT3, CD123, CLL1, CHD3, MSLN, or EpCAM ECD. The “CS1, BCMA, CD20, CD22, FLT3, CD123, CLL1, CHD3, MSLN, or EpCAM extracellular domain” or “CS1, BCMA, CD20, CD22, FLT3, CD123, CLL1, CHD3, MSLN, or EpCAM ECD” refers to the CS1, BCMA, CD20, CD22, FLT3, CD123, CLL1, CHD3, MSLN, or EpCAM region or sequence which is essentially free of transmembrane and cytoplasmic domains of CS1, BCMA, CD20, CD22, FLT3, CD123, CLL1, CHD3, MSLN, or EpCAM. It will be understood by the skilled artisan that the transmembrane domain identified for the CS1, BCMA, CD20, CD22, FLT3, CD123, CLL1, CHD3, MSLN, or EpCAM polypeptide of the present invention is identified pursuant to criteria routinely employed in the art for identifying that type of hydrophobic domain. The exact boundaries of a transmembrane domain may vary but most likely by no more than about 5 amino acids at either end of the domain specifically mentioned herein.

Preferred binding domains which bind to CD3 are disclosed in WO 2010/037836, and WO 2011/121110. Any binding domain for CD3 described in these applications may be used in the context of the present invention.

The invention further provides a polynucleotide/nucleic acid molecule encoding an antigen-binding molecule of the invention. A polynucleotide is a biopolymer composed of 13 or more nucleotide monomers covalently bonded in a chain. DNA (such as cDNA) and RNA (such as mRNA) are examples of polynucleotides with distinct biological function. Nucleotides are organic molecules that serve as the monomers or subunits of nucleic acid molecules like DNA or RNA. The nucleic acid molecule or polynucleotide can be double stranded and single stranded, linear and circular. It is preferably comprised in a vector which is preferably comprised in a host cell. Said host cell is, e.g. after transformation or transfection with the vector or the polynucleotide of the invention, capable of expressing the antigen-binding molecule. For that purpose the polynucleotide or nucleic acid molecule is operatively linked with control sequences.

The genetic code is the set of rules by which information encoded within genetic material (nucleic acids) is translated into proteins. Biological decoding in living cells is accomplished by the ribosome which links amino acids in an order specified by mRNA, using tRNA molecules to carry amino acids and to read the mRNA three nucleotides at a time. The code defines how sequences of these nucleotide triplets, called codons, specify which amino acid will be added next during protein synthesis. With some exceptions, a three-nucleotide codon in a nucleic acid sequence specifies a single amino acid. Because the vast majority of genes are encoded with exactly the same code, this particular code is often referred to as the canonical or standard genetic code. While the genetic code determines the protein sequence for a given coding region, other genomic regions can influence when and where these proteins are produced.

Furthermore, the invention provides a vector comprising a polynucleotide/nucleic acid molecule of the invention. A vector is a nucleic acid molecule used as a vehicle to transfer (foreign) genetic material into a cell. The term “vector” encompasses—but is not restricted to—plasmids, viruses, cosmids and artificial chromosomes. In general, engineered vectors comprise an origin of replication, a multicloning site and a selectable marker. The vector itself is generally a nucleotide sequence, commonly a DNA sequence that comprises an insert (transgene) and a larger sequence that serves as the “backbone” of the vector. Modern vectors may encompass additional features besides the transgene insert and a backbone: promoter, genetic marker, antibiotic resistance, reporter gene, targeting sequence, protein purification tag. Vectors called expression vectors (expression constructs) specifically are for the expression of the transgene in the target cell, and generally have control sequences.

The term “control sequences” refers to DNA sequences necessary for the expression of an operably linked coding sequence in a particular host organism. The control sequences that are suitable for prokaryotes, for example, include a promoter, optionally an operator sequence, and a ribosome binding side. Eukaryotic cells are known to utilize promoters, polyadenylation signals, and enhancers.

A nucleic acid is “operably linked” when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding side is operably linked to a coding sequence if it is positioned so as to facilitate translation. Generally, “operably linked” means that the DNA sequences being linked are contiguous, and, in the case of a secretory leader, contiguous and in reading phase. However, enhancers do not have to be contiguous. Linking is accomplished by ligation at convenient restriction sites. If such sites do not exist, the synthetic oligonucleotide adaptors or linkers are used in accordance with conventional practice.

“Transfection” is the process of deliberately introducing nucleic acid molecules or polynucleotides (including vectors) into target cells. The term is mostly used for non-viral methods in eukaryotic cells. Transduction is often used to describe virus-mediated transfer of nucleic acid molecules or polynucleotides. Transfection of animal cells typically involves opening transient pores or “holes” in the cell membrane, to allow the uptake of material. Transfection can be carried out using calcium phosphate, by electroporation, by cell squeezing or by mixing a cationic lipid with the material to produce liposomes, which fuse with the cell membrane and deposit their cargo inside.

The term “transformation” is used to describe non-viral transfer of nucleic acid molecules or polynucleotides (including vectors) into bacteria, and also into non-animal eukaryotic cells, including plant cells. Transformation is hence the genetic alteration of a bacterial or non-animal eukaryotic cell resulting from the direct uptake through the cell membrane(s) from its surroundings and subsequent incorporation of exogenous genetic material (nucleic acid molecules). Transformation can be effected by artificial means. For transformation to happen, cells or bacteria must be in a state of competence, which may occur as a time-limited response to environmental conditions such as starvation and cell density.

Moreover, the invention provides a host cell transformed or transfected with the polynucleotide/nucleic acid molecule or with the vector of the invention. As used herein, the terms “host cell” or “recipient cell” are intended to include any individual cell or cell culture that can be or has/have been recipients of vectors, exogenous nucleic acid molecules, and polynucleotides encoding the antigen-binding molecule of the present invention; and/or recipients of the antigen-binding molecule itself. The introduction of the respective material into the cell is carried out by way of transformation, transfection and the like. The term “host cell” is also intended to include progeny or potential progeny of a single cell. Because certain modifications may occur in succeeding generations due to either natural, accidental, or deliberate mutation or due to environmental influences, such progeny may not, in fact, be completely identical (in morphology or in genomic or total DNA complement) to the parent cell, but is still included within the scope of the term as used herein. Suitable host cells include prokaryotic or eukaryotic cells, and also include but are not limited to bacteria, yeast cells, fungi cells, plant cells, and animal cells such as insect cells and mammalian cells, e.g., murine, rat, macaque or human.

The antigen-binding molecule of the invention can be produced in bacteria. After expression, the antigen-binding molecule of the invention is isolated from the E. coli cell paste in a soluble fraction and can be purified through, e.g., affinity chromatography and/or size exclusion. Final purification can be carried out similar to the process for purifying antibody expressed e.g., in CHO cells.

In addition to prokaryotes, eukaryotic microbes such as filamentous fungi or yeast are suitable cloning or expression hosts for the antigen-binding molecule of the invention. Saccharomyces cerevisiae, or common baker's yeast, is the most commonly used among lower eukaryotic host microorganisms. However, a number of other genera, species, and strains are commonly available and useful herein, such as Schizosaccharomyces pombe, Kluyveromyces hosts such as K. lactis, K. fragilis (ATCC 12424), K. bulgaricus (ATCC 16045), K. wickeramii (ATCC 24178), K. waltii (ATCC 56500), K. drosophilarum (ATCC 36906), K. thermotolerans, and K. marxianus; yarrowia (EP 402 226); Pichia pastoris (EP 183 070); Candida; Trichoderma reesia (EP 244 234); Neurospora crassa; Schwanniomyces such as Schwanniomyces occidentalis; and filamentous fungi such as Neurospora, Penicillium, Tolypocladium, and Aspergillus hosts such as A. nidulans and A. niger.

Suitable host cells for the expression of glycosylated antigen-binding molecule of the invention are derived from multicellular organisms. Examples of invertebrate cells include plant and insect cells. Numerous baculoviral strains and variants and corresponding permissive insect host cells from hosts such as Spodoptera frugiperda (caterpillar), Aedes aegypti (mosquito), Aedes albopictus (mosquito), Drosophila melanogaster (fruit fly), and Bombyx mori have been identified. A variety of viral strains for transfection are publicly available, e.g., the L-1 variant of Autographa californica NPV and the Bm-5 strain of Bombyx mori NPV, and such viruses may be used as the virus herein according to the present invention, particularly for transfection of Spodoptera frugiperda cells.

Plant cell cultures of cotton, corn, potato, soybean, petunia, tomato, Arabidopsis and tobacco can also be used as hosts. Cloning and expression vectors useful in the production of proteins in plant cell culture are known to those of skill in the art. See e.g. Hiatt et al., Nature (1989) 342: 76-78, Owen et al. (1992) Bio/Technology 10: 790-794, Artsaenko et al. (1995) The Plant J 8: 745-750, and Fecker et al. (1996) Plant Mol Biol 32: 979-986.

However, interest has been greatest in vertebrate cells, and propagation of vertebrate cells in culture (tissue culture) has become a routine procedure. Examples of useful mammalian host cell lines are monkey kidney CV1 line transformed by SV40 (COS-7, ATCC CRL 1651); human embryonic kidney line (293 or 293 cells subcloned for growth in suspension culture, Graham et al., J. Gen Virol. 36: 59 (1977)); baby hamster kidney cells (BHK, ATCC CCL 10); Chinese hamster ovary cells/-DHFR (CHO, Urlaub et al., Proc. Natl. Acad. Sci. USA 77: 4216 (1980)); mouse sertoli cells (TM4, Mather, Biol. Reprod. 23: 243-251 (1980)); monkey kidney cells (CVI ATCC CCL 70); African green monkey kidney cells (VERO-76, ATCC CRL1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2,1413 8065); mouse mammary tumor (MMT 060562, ATCC CCL5 1); TRI cells (Mather et al., Annals N. Y Acad. Sci. (1982) 383: 44-68); MRC 5 cells; FS4 cells; and a human hepatoma line (Hep G2).

In a further embodiment the invention provides a process for the production of an antigen-binding molecule of the invention, said process comprising culturing a host cell of the invention under conditions allowing the expression of the antigen-binding molecule of the invention and recovering the produced antigen-binding molecule from the culture.

As used herein, the term “culturing” refers to the in vitro maintenance, differentiation, growth, proliferation and/or propagation of cells under suitable conditions in a medium. The term “expression” includes any step involved in the production of an antigen-binding molecule of the invention including, but not limited to, transcription, post-transcriptional modification, translation, post-translational modification, and secretion.

When using recombinant techniques, the antigen-binding molecule can be produced intracellularly, in the periplasmic space, or directly secreted into the medium. If the antigen-binding molecule is produced intracellularly, as a first step, the particulate debris, either host cells or lysed fragments, are removed, for example, by centrifugation or ultrafiltration. Carter et al., Bio/Technology 10: 163-167 (1992) describe a procedure for isolating antibodies which are secreted to the periplasmic space of E. coli. Briefly, cell paste is thawed in the presence of sodium acetate (pH 3.5), EDTA, and phenylmethylsulfonylfluoride (PMSF) over about 30 min. Cell debris can be removed by centrifugation. Where the antibody is secreted into the medium, supernatants from such expression systems are generally first concentrated using a commercially available protein concentration filter, for example, an Amicon or Millipore Pellicon ultrafiltration unit. A protease inhibitor such as PMSF may be included in any of the foregoing steps to inhibit proteolysis and antibiotics may be included to prevent the growth of adventitious contaminants.

The antigen-binding molecule of the invention prepared from the host cells can be recovered or purified using, for example, hydroxylapatite chromatography, gel electrophoresis, dialysis, and affinity chromatography. Other techniques for protein purification such as fractionation on an ion-exchange column, ethanol precipitation, Reverse Phase HPLC, chromatography on silica, chromatography on heparin SEPHAROSE™, chromatography on an anion or cation exchange resin (such as a polyaspartic acid column), chromato-focusing, SDS-PAGE, and ammonium sulfate precipitation are also available depending on the antibody to be recovered. Where the antigen-binding molecule of the invention comprises a CH3 domain, the Bakerbond ABX resin (J. T. Baker, Phillipsburg, NJ) is useful for purification.

Affinity chromatography is a preferred purification technique. The matrix to which the affinity ligand is attached is most often agarose, but other matrices are available. Mechanically stable matrices such as controlled pore glass or poly (styrenedivinyl) benzene allow for faster flow rates and shorter processing times than can be achieved with agarose.

Moreover, the invention provides a pharmaceutical composition comprising an antigen-binding molecule of the invention or an antigen-binding molecule produced according to the process of the invention. It is preferred for the pharmaceutical composition of the invention that the homogeneity of the antigen-binding molecule is ≥80%, more preferably ≥81%, ≥82%≥83%≥84%, or ≥85%, further preferably ≥86%, ≥87%, ≥88%≥89%, or ≥90%, still further preferably, ≥91%≥ 92%, ≥93%≥94%, or ≥95% and most preferably ≥96%, ≥97%, ≥98% or ≥99%.

As used herein, the term “pharmaceutical composition” relates to a composition which is suitable for administration to a patient, preferably a human patient. The particularly preferred pharmaceutical composition of this invention comprises one or a plurality of the antigen-binding molecule(s) of the invention, preferably in a therapeutically effective amount. Preferably, the pharmaceutical composition further comprises suitable formulations of one or more (pharmaceutically effective) carriers, stabilizers, excipients, diluents, solubilizers, surfactants, emulsifiers, preservatives and/or adjuvants. Acceptable constituents of the composition are preferably nontoxic to recipients at the dosages and concentrations employed. Pharmaceutical compositions of the invention include, but are not limited to, liquid, frozen, and lyophilized compositions.

The inventive compositions may comprise a pharmaceutically acceptable carrier. In general, as used herein, “pharmaceutically acceptable carrier” means any and all aqueous and non-aqueous solutions, sterile solutions, solvents, buffers, e.g. phosphate buffered saline (PBS) solutions, water, suspensions, emulsions, such as oil/water emulsions, various types of wetting agents, liposomes, dispersion media and coatings, which are compatible with pharmaceutical administration, in particular with parenteral administration. The use of such media and agents in pharmaceutical compositions is well known in the art, and the compositions comprising such carriers can be formulated by well-known conventional methods.

Certain embodiments provide pharmaceutical compositions comprising the antigen-binding molecule of the invention and further one or more excipients such as those illustratively described in this section and elsewhere herein. Excipients can be used in the invention in this regard for a wide variety of purposes, such as adjusting physical, chemical, or biological properties of formulations, such as adjustment of viscosity, and or processes of the invention to improve effectiveness and or to stabilize such formulations and processes against degradation and spoilage due to, for instance, stresses that occur during manufacturing, shipping, storage, pre-use preparation, administration, and thereafter.

In certain embodiments, the pharmaceutical composition may contain formulation materials for the purpose of modifying, maintaining or preserving, e.g., the pH, osmolarity, viscosity, clarity, color, isotonicity, odor, sterility, stability, rate of dissolution or release, adsorption or penetration of the composition (see, REMINGTON'S PHARMACEUTICAL SCIENCES, 18″ Edition, (A. R. Genrmo, ed.), 1990, Mack Publishing Company). In such embodiments, suitable formulation materials may include, but are not limited to:
amino acids such as glycine, alanine, glutamine, asparagine, threonine, proline, 2-phenylalanine, including charged amino acids, preferably lysine, lysine acetate, arginine, glutamate and/or histidine antimicrobials such as antibacterial and antifungal agents
antioxidants such as ascorbic acid, methionine, sodium sulfite or sodium hydrogen-sulfite;
buffers, buffer systems and buffering agents which are used to maintain the composition at physiological pH or at a slightly lower pH, preferably a lower pH of 4.0 to 6.5; examples of buffers are borate, bicarbonate, Tris-HCl, citrates, phosphates or other organic acids, succinate, phosphate, and histidine; for example Tris buffer of about pH 7.0-8.5;
non-aqueous solvents such as propylene glycol, polyethylene glycol, vegetable oils such as olive oil, and injectable organic esters such as ethyl oleate;
aqueous carriers including water, alcoholic/aqueous solutions, emulsions or suspensions, including saline and buffered media;
biodegradable polymers such as polyesters;
bulking agents such as mannitol or glycine;
chelating agents such as ethylenediamine tetraacetic acid (EDTA);
isotonic and absorption delaying agents;
complexing agents such as caffeine, polyvinylpyrrolidone, beta-cyclodextrin or hydroxypropyl-beta-cyclodextrin)
fillers;
monosaccharides; disaccharides; and other carbohydrates (such as glucose, mannose or dextrins); carbohydrates may be non-reducing sugars, preferably trehalose, sucrose, octasulfate, sorbitol or xylitol;
(low molecular weight) proteins, polypeptides or proteinaceous carriers such as human or bovine serum albumin, gelatin or immunoglobulins, preferably of human origin;
coloring and flavouring agents;
sulfur containing reducing agents, such as glutathione, thioctic acid, sodium thioglycolate, thioglycerol, [alpha]-monothioglycerol, and sodium thio sulfate
diluting agents;
emulsifying agents;
hydrophilic polymers such as polyvinylpyrrolidone)
salt-forming counter-ions such as sodium;
preservatives such as antimicrobials, anti-oxidants, chelating agents, inert gases and the like; examples are: benzalkonium chloride, benzoic acid, salicylic acid, thimerosal, phenethyl alcohol, methylparaben, propylparaben, chlorhexidine, sorbic acid or hydrogen peroxide);
metal complexes such as Zn-protein complexes;
solvents and co-solvents (such as glycerin, propylene glycol or polyethylene glycol);

sugars and sugar alcohols, such as trehalose, sucrose, octasulfate, mannitol, sorbitol or xylitol stachyose, mannose, sorbose, xylose, ribose, myoinisitose, galactose, lactitol, ribitol, myoinisitol, galactitol, glycerol, cyclitols (e.g., inositol), polyethylene glycol; and polyhydric sugar alcohols;
suspending agents;
surfactants or wetting agents such as pluronics, PEG, sorbitan esters, polysorbates such as polysorbate 20, polysorbate, triton, tromethamine, lecithin, cholesterol, tyloxapal; surfactants may be detergents, preferably with a molecular weight of >1.2 KD and/or a polyether, preferably with a molecular weight of >3 KD; non-limiting examples for preferred detergents are Tween 20, Tween 40, Tween 60, Tween 80 and Tween 85; non-limiting examples for preferred polyethers are PEG 3000, PEG 3350, PEG 4000 and PEG 5000;
stability enhancing agents such as sucrose or sorbitol;
tonicity enhancing agents such as alkali metal halides, preferably sodium or potassium chloride, mannitol sorbitol;
parenteral delivery vehicles including sodium chloride solution, Ringer's dextrose, dextrose and sodium chloride, lactated Ringer's, or fixed oils;
intravenous delivery vehicles including fluid and nutrient replenishers, electrolyte replenishers (such as those based on Ringer's dextrose).

In the context of the present invention, a pharmaceutical composition, which is preferably a liquid composition or may be a solid composition obtained by lyophilisation or may be a reconstituted liquid composition comprises
(a) an antigen-binding molecule comprising at least four binding domains, wherein:
   a first and a third domain binds to a target cell surface antigen and has an isoelectric point (pI) in the range of 4 to 9,5;
   a second and a fourth domain binds to CD3; and has a pI in the range of 8 to 10, preferably 8.5 to 9.0; and
   a spacer comprising preferably two polypeptide monomers, each comprising a hinge, a CH2 domain and a CH3 domain, wherein said two polypeptide monomers are fused to each other via a peptide linker;
(b) at least one buffer agent;
(c) at least one saccharide; and
(d) at least one surfactant;
and wherein the pH of the pharmaceutical composition is in the range of 3.5 to 6.

It is further envisaged in the context of the present invention that the at least one buffer agent is present at a concentration range of 5 to 200 mM, more preferably at a concentration range of 10 to 50 mM. It is envisaged in the context of the present invention that the at least one saccharide is selected from the group consisting of monosaccharide, disaccharide, cyclic polysaccharide, sugar alcohol, linear branched dextran or linear non-branched dextran. It is also envisaged in the context of the present invention that the disaccharide is selected from the group consisting of sucrose, trehalose and mannitol, sorbitol, and combinations thereof. It is further envisaged in the context of the present invention that the sugar alcohol is sorbitol. It is envisaged in the context of the present invention that the at least one saccharide is present at a concentration in the range of 1 to 15% (m/V), preferably in a concentration range of 9 to 12% (m/V).

It is also envisaged in the context of the present invention that the at least one surfactant is selected from the group consisting of polysorbate 20, polysorbate 40, polysorbate 60, polysorbate 80, poloxamer 188, pluronic F68, triton X-100, polyoxyethylen, PEG 3350, PEG 4000 and combinations thereof. It is further envisaged in the context of the present invention that the at least one surfactant is present at a concentration in the range of 0.004 to 0.5% (m/V), preferably in the range of 0.001 to 0.01% (m/V). It is envisaged in the context of the present invention that the pH of the composition is in the range of 4.0 to 5.0, preferably 4.2. It is also envisaged in the context of the present invention that the pharmaceutical composition has an osmolarity in the range of 150 to 500 mOsm. It is further envisaged in the context of the present invention that the pharmaceutical composition further comprises an excipient selected from the group consisting of, one or more polyol and one or more amino acid. It is envisaged in the context of the present invention that said one or more excipient is present in the concentration range of 0.1 to 15% (w/V).

It is also envisaged in the context of the present invention that the pharmaceutical composition comprises
(a) the antigen-binding molecule as discussed above,
(b) 10 mM glutamate or acetate,
(c) 9% (m/V) sucrose or 6% (m/V) sucrose and 6% (m/V) hydroxypropyl-β-cyclodextrin,
(d) 0.01% (m/V) polysorbate 80
and wherein the pH of the liquid pharmaceutical composition is 4.2.

It is further envisaged in the context of the present invention that the antigen-binding molecule is present in a concentration range of 0.1 to 8 mg/ml, preferably of 0.2-2.5 mg/ml, more preferably of 0.25-1.0 mg/ml.

It is evident to those skilled in the art that the different constituents of the pharmaceutical composition (e.g., those listed above) can have different effects, for example, and amino acid can act as a buffer, a stabilizer and/or an antioxidant; mannitol can act as a bulking agent and/or a tonicity enhancing agent; sodium chloride can act as delivery vehicle and/or tonicity enhancing agent; etc.

It is envisaged that the composition of the invention may comprise, in addition to the polypeptide of the invention defined herein, further biologically active agents, depending on the intended use of the composition. Such agents may be drugs acting on the gastro-intestinal system, drugs acting as cytostatica, drugs preventing hyperurikemia, drugs inhibiting immunoreactions (e.g. corticosteroids), drugs modulating the inflammatory response, drugs acting on the circulatory system and/or agents such as cytokines known in the art. It is also envisaged that the antigen-binding molecule of the present invention is applied in a co-therapy, i.e., in combination with another anti-cancer medicament.

In certain embodiments, optimal pharmaceutical compositions may influence the physical state, stability, rate of in vivo release and rate of in vivo clearance of the antigen-binding molecule of the invention. In certain embodiments, the primary vehicle or carrier in a pharmaceutical composition may be either aqueous or non-aqueous in nature. For example, a suitable vehicle or carrier may be water for injection, physiological saline solution or artificial cerebrospinal fluid, possibly supplemented with other materials common in compositions for parenteral administration. Neutral buffered saline or saline mixed with serum albumin are further exemplary vehicles. In certain embodiments, the antigen-binding molecule of the invention compositions may be prepared for storage by mixing the selected composition having the desired degree of purity with optional formulation agents (REMINGTON'S PHARMACEUTICAL SCIENCES, supra) in the form of a lyophilized cake or an aqueous solution. Further, in certain embodiments, the antigen-binding molecule of the invention may be formulated as a lyophilizate using appropriate excipients such as sucrose.

When parenteral administration is contemplated, the therapeutic compositions for use in this invention may be provided in the form of a pyrogen-free, parenterally acceptable aqueous solution comprising the desired antigen-binding molecule of the invention in a pharmaceutically acceptable vehicle. A particularly suitable vehicle for parenteral injection is sterile distilled water in which the antigen-binding molecule of the invention is formulated as a sterile, isotonic solution, properly preserved. In certain embodiments, the preparation can involve the formulation of the desired molecule with an agent, such as injectable microspheres, bio-erodible particles, polymeric compounds (such as polylactic acid or polyglycolic acid), beads or liposomes, that may provide controlled or sustained release of the product which can be delivered via depot injection. In certain embodiments, hyaluronic acid may also be used, having the effect of promoting sustained duration in the circulation. In certain embodiments, implantable drug delivery devices may be used to introduce the desired antigen-binding molecule.

Additional pharmaceutical compositions will be evident to those skilled in the art, including formulations involving the antigen-binding molecule of the invention in sustained- or controlled-delivery/release formulations. Techniques for formulating a variety of other sustained- or controlled-delivery means, such as liposome carriers, bio-erodible microparticles or porous beads and depot injections, are also known to those skilled in the art. See, for example, International Patent Application No. PCT/US93/00829, which describes controlled release of porous polymeric microparticles for delivery of pharmaceutical compositions. Sustained-release preparations may include semipermeable polymer matrices in the form of shaped articles, e.g., films, or microcapsules. Sustained release matrices may include polyesters, hydrogels, polylactides (as disclosed in U.S. Pat. No. 3,773,919 and European Patent Application Publication No. EP 058481), copolymers of L-glutamic acid and gamma ethyl-L-glutamate (Sidman et al., 1983, Biopolymers 2:547-556), poly (2-hydroxyethyl-methacrylate) (Langer et al., 1981, J. Biomed. Mater. Res. 15:167-277 and Langer, 1982, Chem. Tech. 12:98-105), ethylene vinyl acetate (Langer et al., 1981, supra) or poly-D(−)-3-hydroxybutyric acid (European Patent Application Publication No. EP 133,988). Sustained release compositions may also include liposomes that can be prepared by any of several methods known in the art. See, e.g., Eppstein et al., 1985, Proc. Natl. Acad. Sci. U.S.A. 82:3688-3692; European Patent Application Publication Nos. EP 036,676; EP 088,046 and EP 143,949.

The antigen-binding molecule may also be entrapped in microcapsules prepared, for example, by coacervation techniques or by interfacial polymerization (for example, hydroxymethylcellulose or gelatine-microcapsules and poly (methylmethacylate) microcapsules, respectively), in colloidal drug delivery systems (for example, liposomes, albumin microspheres, microemulsions, nanoparticles and nanocapsules), or in macroemulsions. Such techniques are disclosed in Remington's Pharmaceutical Sciences, 16th edition, Oslo, A., Ed., (1980).

Pharmaceutical compositions used for in vivo administration are typically provided as sterile preparations. Sterilization can be accomplished by filtration through sterile filtration membranes. When the composition is lyophilized, sterilization using this method may be conducted either prior to or following lyophilization and reconstitution. Compositions for parenteral administration can be stored in lyophilized form or in a solution. Parenteral compositions generally are placed into a container having a sterile access port, for example, an intravenous solution bag or vial having a stopper pierceable by a hypodermic injection needle.

Another aspect of the invention includes self-buffering antigen-binding molecule of the invention formulations, which can be used as pharmaceutical compositions, as described in international patent application WO 06138181A2 (PCT/US2006/022599). A variety of expositions are available on protein stabilization and formulation materials and methods useful in this regard, such as Arakawa et al., “Solvent interactions in pharmaceutical formulations,” Pharm Res. 8(3): 285-91 (1991); Kendrick et al., “Physical stabilization of proteins in aqueous solution” in: RATIONAL DESIGN OF STABLE PROTEIN FORMULATIONS: THEORY AND PRACTICE, Carpenter and Manning, eds. Pharmaceutical Biotechnology. 13: 61-84 (2002), and Randolph et al., “Surfactant-protein interactions”. Pharm Biotechnol. 13: 159-75 (2002), see particularly the parts pertinent to excipients and processes of the same for self-buffering protein formulations in accordance with the current invention, especially as to protein pharmaceutical products and processes for veterinary and/or human medical uses.

Salts may be used in accordance with certain embodiments of the invention to, for example, adjust the ionic strength and/or the isotonicity of a formulation and/or to improve the solubility and/or physical stability of a protein or other ingredient of a composition in accordance with the invention. As is well known, ions can stabilize the native state of proteins by binding to charged residues on the protein's surface and by shielding charged and polar groups in the protein and reducing the strength of their electrostatic interactions, attractive, and repulsive interactions. Ions also can stabilize the denatured state of a protein by binding to, in particular, the denatured peptide linkages (—CONH) of the protein. Furthermore, ionic interaction with charged and polar groups in a protein also can reduce intermolecular electrostatic interactions and, thereby, prevent or reduce protein aggregation and insolubility.

Ionic species differ significantly in their effects on proteins. A number of categorical rankings of ions and their effects on proteins have been developed that can be used in formulating pharmaceutical compositions in accordance with the invention. One example is the Hofmeister series, which ranks ionic and polar non-ionic solutes by their effect on the conformational stability of proteins in solution. Stabilizing solutes are referred to as “kosmotropic”. Destabilizing solutes are referred to as “chaotropic”. Kosmotropes commonly are used at high concentrations (e.g., >1 molar ammonium sulfate) to precipitate proteins from solution (“salting-out”). Chaotropes commonly are used to denture and/or to solubilize proteins (“salting-in”). The relative effectiveness of ions to “salt-in” and “salt-out” defines their position in the Hofmeister series.

Free amino acids can be used in the antigen-binding molecule of the invention formulations in accordance with various embodiments of the invention as bulking agents, stabilizers, and antioxidants, as well as other standard uses. Lysine, proline, serine, and alanine can be used for stabilizing proteins in a formulation. Glycine is useful in lyophilization to ensure correct cake structure and properties. Arginine may be useful to inhibit protein aggregation, in both liquid and lyophilized formulations. Methionine is useful as an antioxidant.

Polyols include sugars, e.g., mannitol, sucrose, and sorbitol and polyhydric alcohols such as, for instance, glycerol and propylene glycol, and, for purposes of discussion herein, polyethylene glycol (PEG) and related substances. Polyols are kosmotropic. They are useful stabilizing agents in both liquid and lyophilized formulations to protect proteins from physical and chemical degradation processes. Polyols also are useful for adjusting the tonicity of formulations. Among polyols useful in select embodiments of the invention is mannitol, commonly used to ensure structural stability of the cake in lyophilized formulations. It ensures structural stability to the cake. It is generally used with a lyoprotectant, e.g., sucrose. Sorbitol and sucrose are among preferred agents for adjusting tonicity and as stabilizers to protect against freeze-thaw stresses during transport or the preparation of bulks during the manufacturing process. Reducing sugars (which contain free aldehyde or ketone groups), such as glucose and lactose, can glycate surface lysine and arginine residues. Therefore, they generally are not among preferred polyols for use in accordance with the invention. In addition, sugars that form such reactive species, such as sucrose, which is hydrolyzed to fructose and glucose under acidic conditions, and consequently engenders glycation, also is not among preferred polyols of the invention in this regard. PEG is useful to stabilize proteins and as a cryoprotectant and can be used in the invention in this regard.

Embodiments of the antigen-binding molecule of the invention formulations further comprise surfactants. Protein molecules may be susceptible to adsorption on surfaces and to denaturation and consequent aggregation at air-liquid, solid-liquid, and liquid-liquid interfaces. These effects generally scale inversely with protein concentration. These deleterious interactions generally scale inversely with protein concentration and typically are exacerbated by physical agitation, such as that generated during the shipping and handling of a product. Surfactants routinely are used to prevent, minimize, or reduce surface adsorption. Useful surfactants in the invention in this regard include polysorbate 20, polysorbate 80, other fatty acid esters of sorbitan polyethoxylates, and poloxamer 188. Surfactants also are commonly used to control protein conformational stability. The use of surfactants in this regard is protein-specific since, any given surfactant typically will stabilize some proteins and destabilize others.

Polysorbates are susceptible to oxidative degradation and often, as supplied, contain sufficient quantities of peroxides to cause oxidation of protein residue side-chains, especially methionine. Consequently, polysorbates should be used carefully, and when used, should be employed at their lowest effective concentration. In this regard, polysorbates exemplify the general rule that excipients should be used in their lowest effective concentrations.

Embodiments of the antigen-binding molecule of the invention formulations further comprise one or more antioxidants. To some extent deleterious oxidation of proteins can be prevented in pharmaceutical formulations by maintaining proper levels of ambient oxygen and temperature and by avoiding exposure to light. Antioxidant excipients can be used as well to prevent oxidative degradation of proteins. Among useful antioxidants in this regard are reducing agents, oxygen/free-radical scavengers, and chelating agents. Antioxidants for use in therapeutic protein formulations in accordance with the invention preferably are water-soluble and maintain their activity throughout the shelf life of a product. EDTA is a preferred antioxidant in accordance with the invention in this regard. Antioxidants can damage proteins. For instance, reducing agents, such as glutathione in particular, can disrupt intramolecular disulfide linkages. Thus, antioxidants for use in the invention are selected to, among other things, eliminate or sufficiently reduce the possibility of themselves damaging proteins in the formulation.

Formulations in accordance with the invention may include metal ions that are protein co-factors and that are necessary to form protein coordination complexes, such as zinc necessary to form certain insulin suspensions. Metal ions also can inhibit some processes that degrade proteins. However, metal ions also catalyze physical and chemical processes that degrade proteins. Magnesium ions (10-120 mM) can be used to inhibit isomerization of aspartic acid to isoaspartic acid. Ca⁺² ions (up to 100 mM) can increase the stability of human deoxyribonuclease. Mg⁺², Mn⁺², and Zn⁺², however, can destabilize rhDNase. Similarly, Ca⁺² and Sr⁺² can stabilize Factor VIII, it can be destabilized by Mg⁺², Mn⁺² and Zn⁺², Cu⁺² and Fe⁺², and its aggregation can be increased by Al⁺³ ions.

Embodiments of the antigen-binding molecule of the invention formulations further comprise one or more preservatives. Preservatives are necessary when developing multi-dose parenteral formulations that involve more than one extraction from the same container. Their primary function is to inhibit microbial growth and ensure product sterility throughout the shelf-life or term of use of the drug product. Commonly used preservatives include benzyl alcohol, phenol and m-cresol. Although preservatives have a long history of use with small-molecule parenterals, the development of protein formulations that includes preservatives can be challenging. Preservatives almost always have a destabilizing effect (aggregation) on proteins, and this has become a major factor in limiting their use in multi-dose protein formulations. To date, most protein drugs have been formulated for single-use only. However, when multi-dose formulations are possible, they have the added advantage of enabling patient convenience, and increased marketability. A good example is that of human growth hormone (hGH) where the development of preserved formulations has led to commercialization of more convenient, multi-use injection pen presentations. At least four such pen devices containing preserved formulations of hGH are currently available on the market. Norditropin (liquid, Novo Nordisk), Nutropin AQ (liquid, Genentech) & Genotropin (lyophilized-dual chamber cartridge, Pharmacia & Upjohn) contain phenol while Somatrope (Eli Lilly) is formulated with m-cresol. Several aspects need to be considered during the formulation and development of preserved dosage forms. The effective preservative concentration in the drug product must be optimized. This requires testing a given preservative in the dosage form with concentration ranges that confer anti-microbial effectiveness without compromising protein stability.

As may be expected, development of liquid formulations containing preservatives are more challenging than lyophilized formulations. Freeze-dried products can be lyophilized without the preservative and reconstituted with a preservative containing diluent at the time of use. This shortens the time for which a preservative is in contact with the protein, significantly minimizing the associated stability risks. With liquid formulations, preservative effectiveness and stability should be maintained over the entire product shelf-life (about 18 to 24 months). An important point to note is that preservative effectiveness should be demonstrated in the final formulation containing the active drug and all excipient components.

The antigen-binding molecules disclosed herein may also be formulated as immuno-liposomes. A “liposome” is a small vesicle composed of various types of lipids, phospholipids and/or surfactant which is useful for delivery of a drug to a mammal. The components of the liposome are commonly arranged in a bilayer formation, similar to the lipid arrangement of biological membranes. Liposomes containing the antigen-binding molecule are prepared by methods known in the art, such as described in Epstein et al., Proc. Natl. Acad. Sci. USA, 82: 3688 (1985); Hwang et al., Proc. Natl Acad. Sci. USA, 77: 4030 (1980); U.S. Pat. Nos. 4,485,045 and 4,544,545; and WO 97/38731. Liposomes with enhanced circulation time are disclosed in U.S. Pat. No. 5,013,556. Particularly useful liposomes can be generated by the reverse phase evaporation method with a lipid composition comprising phosphatidylcholine, cholesterol and PEG-derivatized phosphatidylethanolamine (PEG-PE). Liposomes are extruded through filters of defined pore size to yield liposomes with the desired diameter. Fab′ fragments of the antigen-binding molecule of the present invention can be conjugated to the liposomes as described in Martin et al. J. Biol. Chem. 257: 286-288 (1982) via a disulfide interchange reaction. A chemotherapeutic agent is optionally contained within the liposome. See Gabizon et al. J. National Cancer Inst. 81 (19) 1484 (1989).

Once the pharmaceutical composition has been formulated, it may be stored in sterile vials as a solution, suspension, gel, emulsion, solid, crystal, or as a dehydrated or lyophilized powder. Such formulations may be stored either in a ready-to-use form or in a form (e.g., lyophilized) that is reconstituted prior to administration.

The biological activity of the pharmaceutical composition defined herein can be determined for instance by cytotoxicity assays, as described in the following examples, in WO 99/54440 or by Schlereth et al. (Cancer Immunol. Immunother. 20 (2005), 1-12). “Efficacy” or “in vivo efficacy” as used herein refers to the response to therapy by the pharmaceutical composition of the invention, using e.g. standardized NCI response criteria. The success or in vivo efficacy of the therapy using a pharmaceutical composition of the invention refers to the effectiveness of the composition for its intended purpose, i.e. the ability of the composition to cause its desired effect, i.e. depletion of pathologic cells, e.g. tumor cells. The in vivo efficacy may be monitored by established standard methods for the respective disease entities including, but not limited to white blood cell counts, differentials, Fluorescence Activated Cell Sorting, bone marrow aspiration. In addition, various disease specific clinical chemistry parameters and other established standard methods may be used. Furthermore, computer-aided tomography, X-ray, nuclear magnetic resonance tomography (e.g. for National Cancer Institute-criteria based response assessment [Cheson B D, Horning S J, Coiffier B, Shipp M A, Fisher R I, Connors J M, Lister T A, Vose J, Grillo-Lopez A, Hagenbeek A, Cabanillas F, Klippensten D. Hiddemann W, Castellino R, Harris N L, Armitage J O, Carter W, Hoppe R, Canellos G P. Report of an international workshop to standardize response criteria for non-Hodgkin's lymphomas. NCI Sponsored International Working Group. J Clin Oncol. 1999 April; 17(4): 1244]), positron-emission tomography scanning, white blood cell counts, differentials, Fluorescence Activated Cell Sorting, bone marrow aspiration, lymph node biopsies/histologies, and various lymphoma specific clinical chemistry parameters (e.g. lactate dehydrogenase) and other established standard methods may be used.

Another major challenge in the development of drugs such as the pharmaceutical composition of the invention is the predictable modulation of pharmacokinetic properties. To this end, a pharmacokinetic profile of the drug candidate, i.e. a profile of the pharmacokinetic parameters that affect the ability of a particular drug to treat a given condition, can be established. Pharmacokinetic parameters of the drug influencing the ability of a drug for treating a certain disease entity include, but are not limited to: half-life, volume of distribution, hepatic first-pass metabolism and the degree of blood serum binding. The efficacy of a given drug agent can be influenced by each of the parameters mentioned above. It is an envisaged characteristic of the antigen-binding molecules of the present invention provided with the specific FC modality that they comprise, for example, differences in pharmacokinetic behavior. A half-life extended targeting antigen-binding molecule according to the present invention preferably shows a surprisingly increased residence time in vivo in comparison to “canonical” non-HLE versions of said antigen-binding molecule.

“Half-life” means the time where 50% of an administered drug are eliminated through biological processes, e.g. metabolism, excretion, etc. By “hepatic first-pass metabolism” is meant the propensity of a drug to be metabolized upon first contact with the liver, i.e. during its first pass through the liver. “Volume of distribution” means the degree of retention of a drug throughout the various compartments of the body, like e.g. intracellular and extracellular spaces, tissues and organs, etc. and the distribution of the drug within these compartments. “Degree of blood serum binding” means the propensity of a drug to interact with and bind to blood serum proteins, such as albumin, leading to a reduction or loss of biological activity of the drug.

Pharmacokinetic parameters also include bioavailability, lag time (Tlag), Tmax, absorption rates, more onset and/or Cmax for a given amount of drug administered. “Bioavailability” means the amount of a drug in the blood compartment. “Lag time” means the time delay between the administration of the drug and its detection and measurability in blood or plasma. “Tmax” is the time after which maximal blood concentration of the drug is reached, and “Cmax” is the blood concentration maximally obtained with a given drug. The time to reach a blood or tissue concentration of the drug which is required for its biological effect is influenced by all parameters. Pharmacokinetic parameters of bispecific antigen-binding molecules exhibiting cross-species specificity, which may be determined in preclinical animal testing in non-chimpanzee primates as outlined above, are also set forth e.g. in the publication by Schlereth et al. (Cancer Immunol. Immunother. 20 (2005), 1-12).

In a preferred aspect of the invention the pharmaceutical composition is stable for at least four weeks at about −20° C. As apparent from the appended examples the quality of an antigen-binding molecule of the invention vs. the quality of corresponding state of the art antigen-binding molecules may be tested using different systems. Those tests are understood to be in line with the “ICH Harmonised Tripartite Guideline: Stability Testing of Biotechnological/Biological Products Q5C and Specifications: Test procedures and Acceptance Criteria for Biotech Biotechnological/Biological Products Q6B” and, thus are elected to provide a stability-indicating profile that provides certainty that changes in the identity, purity and potency of the product are detected. It is well accepted that the term purity is a relative term. Due to the effect of glycosylation, deamidation, or other heterogeneities, the absolute purity of a biotechnological/biological product should be typically assessed by more than one method and the purity value derived is method-dependent. For the purpose of stability testing, tests for purity should focus on methods for determination of degradation products.

For the assessment of the quality of a pharmaceutical composition comprising an antigen-binding molecule of the invention may be analyzed e.g. by analyzing the content of soluble aggregates in a solution (HMWS per size exclusion). It is preferred that stability for at least four weeks at about −20° C. is characterized by a content of less than 1.5% HMWS, preferably by less than 1% HMWS.

A preferred formulation for the antigen-binding molecule as a pharmaceutical composition may e.g. comprise the components of a formulation as described below:
Formulation:
   potassium phosphate, L-arginine hydrochloride, trehalose dihydrate, polysorbate 80 at pH 6.0

Other examples for the assessment of the stability of an antigen-binding molecule of the invention in form of a pharmaceutical composition are provided in the appended examples 4-12. In those examples embodiments of antigen-binding molecules of the invention are tested with respect to different stress conditions in different pharmaceutical formulations and the results compared with other half-life extending (HLE) formats of bispecific T cell engaging antigen-binding molecule known from the art. In general, it is envisaged that antigen-binding molecules provided with the specific FC modality according to the present invention are typically more stable over a broad range of stress conditions such as temperature and light stress, both compared to antigen-binding molecules provided with different HLE formats and without any HLE format (e.g. “canonical” antigen-binding molecules). Said temperature stability may relate both to decreased (below room temperature including freezing) and increased (above room temperature including temperatures up to or above body temperature) temperature. As the person skilled in the art will acknowledge, such improved stability with regard to stress, which is hardly avoidable in clinical practice, makes the antigen-binding molecule safer because less degradation products will occur in clinical practice. In consequence, said increased stability means increased safety.

One embodiment provides the antigen-binding molecule of the invention or the antigen-binding molecule produced according to the process of the invention for use in the prevention, treatment or amelioration of a cancer correlating with, CD20, CD22, FLT3, CLL1, CHD3, MSLN, or EpCAM expression or CD20, CD22, FLT3, CLL1, CHD3, MSLN, or EpCAM overexpression, such as prostate cancer.

The formulations described herein are useful as pharmaceutical compositions in the treatment, amelioration and/or prevention of the pathological medical condition as described herein in a patient in need thereof. The term “treatment” refers to both therapeutic treatment and prophylactic or preventative measures. Treatment includes the application or administration of the formulation to the body, an isolated tissue, or cell from a patient who has a disease/disorder, a symptom of a disease/disorder, or a predisposition toward a disease/disorder, with the purpose to cure, heal, alleviate, relieve, alter, remedy, ameliorate, improve, or affect the disease, the symptom of the disease, or the predisposition toward the disease.

The term “amelioration” as used herein refers to any improvement of the disease state of a patient having a disease as specified herein below, by the administration of an antigen-binding molecule according to the invention to a subject in need thereof. Such an improvement may also be seen as a slowing or stopping of the progression of the patient's disease. The term “prevention” as used herein means the avoidance of the occurrence or re-occurrence of a patient having a tumor or cancer or a metastatic cancer as specified herein below, by the administration of an antigen-binding molecule according to the invention to a subject in need thereof.

The term “disease” refers to any condition that would benefit from treatment with the antigen-binding molecule or the pharmaceutic composition described herein. This includes chronic and acute disorders or diseases including those pathological conditions that predispose the mammal to the disease in question.

A “neoplasm” is an abnormal growth of tissue, usually but not always forming a mass. When also forming a mass, it is commonly referred to as a “tumor”. Neoplasms or tumors or can be benign, potentially malignant (pre-cancerous), or malignant. Malignant neoplasms are commonly called cancer. They usually invade and destroy the surrounding tissue and may form metastases, i.e., they spread to other parts, tissues or organs of the body. Hence, the term “metastatic cancer” encompasses metastases to other tissues or organs than the one of the original tumor. Lymphomas and leukemias are lymphoid neoplasms. For the purposes of the present invention, they are also encompassed by the terms “tumor” or “cancer”.

The term “viral disease” describes diseases, which are the result of a viral infection of a subject.

The term “immunological disorder” as used herein describes in line with the common definition of this term immunological disorders such as autoimmune diseases, hypersensitivities, immune deficiencies.

In one embodiment the invention provides a method for the treatment or amelioration of a cancer correlating with CS1, BCMA, CD20, CD22, FLT3, CD123, CLL1, CHD3, MSLN, or EpCAM expression or CS1, BCMA, CD20, CD22, FLT3, CD123, CLL1, CHD3, MSLN, or EpCAM overexpression, comprising the step of administering to a subject in need thereof the antigen-binding molecule of the invention, or the antigen-binding molecule produced according to the process of the invention. The CS1, BCMA, CD20, CD22, FLT3, CD123, CLL1, CHD3, MSLN, or EpCAMxCD3 bispecific single chain antibody is particularly advantageous for the therapy of cancer, preferably solid tumors, more preferably carcinomas and prostate cancer.

The terms “subject in need” or those “in need of treatment” includes those already with the disorder, as well as those in which the disorder is to be prevented. The subject in need or “patient” includes human and other mammalian subjects that receive either prophylactic or therapeutic treatment.

The antigen-binding molecule of the invention will generally be designed for specific routes and methods of administration, for specific dosages and frequencies of administration, for specific treatments of specific diseases, with ranges of bio-availability and persistence, among other things. The materials of the composition are preferably formulated in concentrations that are acceptable for the site of administration.

Formulations and compositions thus may be designed in accordance with the invention for delivery by any suitable route of administration. In the context of the present invention, the routes of administration include, but are not limited to
topical routes (such as epicutaneous, inhalational, nasal, opthalmic, auricular/aural, vaginal, mucosal);
enteral routes (such as oral, gastrointestinal, sublingual, sublabial, buccal, rectal); and
parenteral routes (such as intravenous, intraarterial, intraosseous, intramuscular, intracerebral, intracerebroventricular, epidural, intrathecal, subcutaneous, intraperitoneal, extra-amniotic, intraarticular, intracardiac, intradermal, intralesional, intrauterine, intravesical, intravitreal, transdermal, intranasal, transmucosal, intrasynovial, intraluminal).

The pharmaceutical compositions and the antigen-binding molecule of this invention are particularly useful for parenteral administration, e.g., subcutaneous or intravenous delivery, for example by injection such as bolus injection, or by infusion such as continuous infusion. Pharmaceutical compositions may be administered using a medical device. Examples of medical devices for administering pharmaceutical compositions are described in U.S. Pat. Nos. 4,475,196; 4,439,196; 4,447,224; 4,447, 233; 4,486,194; 4,487,603; 4,596,556; 4,790,824; 4,941,880; 5,064,413; 5,312,335; 5,312,335; 5,383,851; and 5,399,163.

In particular, the present invention provides for an uninterrupted administration of the suitable composition. As a non-limiting example, uninterrupted or substantially uninterrupted, i.e. continuous administration may be realized by a small pump system worn by the patient for metering the influx of therapeutic agent into the body of the patient. The pharmaceutical composition comprising the antigen-binding molecule of the invention can be administered by using said pump systems. Such pump systems are generally known in the art, and commonly rely on periodic exchange of cartridges containing the therapeutic agent to be infused. When exchanging the cartridge in such a pump system, a temporary interruption of the otherwise uninterrupted flow of therapeutic agent into the body of the patient may ensue. In such a case, the phase of administration prior to cartridge replacement and the phase of administration following cartridge replacement would still be considered within the meaning of the pharmaceutical means and methods of the invention together make up one “uninterrupted administration” of such therapeutic agent.

The continuous or uninterrupted administration of the antigen-binding molecules of the invention may be intravenous or subcutaneous by way of a fluid delivery device or small pump system including a fluid driving mechanism for driving fluid out of a reservoir and an actuating mechanism for actuating the driving mechanism. Pump systems for subcutaneous administration may include a needle or a cannula for penetrating the skin of a patient and delivering the suitable composition into the patient's body. Said pump systems may be directly fixed or attached to the skin of the patient independently of a vein, artery or blood vessel, thereby allowing a direct contact between the pump system and the skin of the patient. The pump system can be attached to the skin of the patient for 24 hours up to several days. The pump system may be of small size with a reservoir for small volumes. As a non-limiting example, the volume of the reservoir for the suitable pharmaceutical composition to be administered can be between 0.1 and 50 ml.

The continuous administration may also be transdermal by way of a patch worn on the skin and replaced at intervals. One of skill in the art is aware of patch systems for drug delivery suitable for this purpose. It is of note that transdermal administration is especially amenable to uninterrupted administration, as exchange of a first exhausted patch can advantageously be accomplished simultaneously with the placement of a new, second patch, for example on the surface of the skin immediately adjacent to the first exhausted patch and immediately prior to removal of the first exhausted patch. Issues of flow interruption or power cell failure do not arise.

If the pharmaceutical composition has been lyophilized, the lyophilized material is first reconstituted in an appropriate liquid prior to administration. The lyophilized material may be reconstituted in, e.g., bacteriostatic water for injection (BWFI), physiological saline, phosphate buffered saline (PBS), or the same formulation the protein had been in prior to lyophilization.

The compositions of the present invention can be administered to the subject at a suitable dose which can be determined e.g. by dose escalating studies by administration of increasing doses of the antigen-binding molecule of the invention exhibiting cross-species specificity described herein to non-chimpanzee primates, for instance macaques. As set forth above, the antigen-binding molecule of the invention exhibiting cross-species specificity described herein can be advantageously used in identical form in preclinical testing in non-chimpanzee primates and as drug in humans.

The term “effective dose” or “effective dosage” is defined as an amount sufficient to achieve or at least partially achieve the desired effect. The term “therapeutically effective dose” is defined as an amount sufficient to cure or at least partially arrest the disease and its complications in a patient already suffering from the disease. Amounts or doses effective for this use will depend on the condition to be treated (the indication), the delivered antigen-binding molecule, the therapeutic context and objectives, the severity of the disease, prior therapy, the patient's clinical history and response to the therapeutic agent, the route of administration, the size (body weight, body surface or organ size) and/or condition (the age and general health) of the patient, and the general state of the patient's own immune system.

A typical dosage may range from about 0.1 μg/kg to up to about 30 mg/kg or more, depending on the factors mentioned above. In specific embodiments, the dosage may range from 1.0 μg/kg up to about 20 mg/kg, optionally from 10 μg/kg up to about 10 mg/kg or from 100 μg/kg up to about 5 mg/kg.

A therapeutic effective amount of an antigen-binding molecule of the invention preferably results in a decrease in severity of disease symptoms, an increase in frequency or duration of disease symptom-free periods or a prevention of impairment or disability due to the disease affliction. For treating diseases correlating with CS1, BCMA, CD20, CD22, FLT3, CD123, CLL1, CHD3, MSLN, or EpCAM expression as described herein above, a therapeutically effective amount of the antigen-binding molecule of the invention, here: an anti-CS1, BCMA, CD20, CD22, FLT3, CD123, CLL1, CHD3, MSLN, or EpCAM/anti-CD3 antigen-binding molecule, preferably inhibits cell growth or tumor growth by at least about 20%, at least about 40%, at least about 50%, at least about 60%, at least about 70%, at least about 80%, or at least about 90% relative to untreated patients. The ability of a compound to inhibit tumor growth may be evaluated in an animal model predictive of efficacy

The pharmaceutical composition can be administered as a sole therapeutic or in combination with additional therapies such as anti-cancer therapies as needed, e.g. other proteinaceous and non-proteinaceous drugs. These drugs may be administered simultaneously with the composition comprising the antigen-binding molecule of the invention as defined herein or separately before or after administration of said antigen-binding molecule in timely defined intervals and doses.

The term “effective and non-toxic dose” as used herein refers to a tolerable dose of an inventive antigen-binding molecule which is high enough to cause depletion of pathologic cells, tumor elimination, tumor shrinkage or stabilization of disease without or essentially without major toxic effects. Such effective and non-toxic doses may be determined e.g. by dose escalation studies described in the art and should be below the dose inducing severe adverse side events (dose limiting toxicity, DLT).

The term “toxicity” as used herein refers to the toxic effects of a drug manifested in adverse events or severe adverse events. These side events may refer to a lack of tolerability of the drug in general and/or a lack of local tolerance after administration. Toxicity could also include teratogenic or carcinogenic effects caused by the drug.

The term “safety”, “in vivo safety” or “tolerability” as used herein defines the administration of a drug without inducing severe adverse events directly after administration (local tolerance) and during a longer period of application of the drug. “Safety”, “in vivo safety” or “tolerability” can be evaluated e.g. at regular intervals during the treatment and follow-up period. Measurements include clinical evaluation, e.g. organ manifestations, and screening of laboratory abnormalities. Clinical evaluation may be carried out and deviations to normal findings recorded/coded according to NCI-CTC and/or MedDRA standards. Organ manifestations may include criteria such as allergy/immunology, blood/bone marrow, cardiac arrhythmia, coagulation and the like, as set forth e.g. in the Common Terminology Criteria for adverse events v3.0 (CTCAE). Laboratory parameters which may be tested include for instance hematology, clinical chemistry, coagulation profile and urine analysis and examination of other body fluids such as serum, plasma, lymphoid or spinal fluid, liquor and the like. Safety can thus be assessed e.g. by physical examination, imaging techniques (i.e. ultrasound, x-ray, CT scans, Magnetic Resonance Imaging (MRI), other measures with technical devices (i.e. electrocardiogram), vital signs, by measuring laboratory parameters and recording adverse events. For example, adverse events in non-chimpanzee primates in the uses and methods according to the invention may be examined by histopathological and/or histochemical methods.

The above terms are also referred to e.g. in the Preclinical safety evaluation of biotechnology-derived pharmaceuticals S6; ICH Harmonised Tripartite Guideline; ICH Steering Committee meeting on Jul. 16, 1997.

Finally, the invention provides a kit comprising an antigen-binding molecule of the invention or produced according to the process of the invention, a pharmaceutical composition of the invention, a polynucleotide of the invention, a vector of the invention and/or a host cell of the invention.

In the context of the present invention, the term “kit” means two or more components—one of which corresponding to the antigen-binding molecule, the pharmaceutical composition, the vector or the host cell of the invention—packaged together in a container, recipient or otherwise. A kit can hence be described as a set of products and/or utensils that are sufficient to achieve a certain goal, which can be marketed as a single unit.

The kit may comprise one or more recipients (such as vials, ampoules, containers, syringes, bottles, bags) of any appropriate shape, size and material (preferably waterproof, e.g. plastic or glass) containing the antigen-binding molecule or the pharmaceutical composition of the present invention in an appropriate dosage for administration (see above). The kit may additionally contain directions for use (e.g. in the form of a leaflet or instruction manual), means for administering the antigen-binding molecule of the present invention such as a syringe, pump, infuser or the like, means for reconstituting the antigen-binding molecule of the invention and/or means for diluting the antigen-binding molecule of the invention.

The invention also provides kits for a single-dose administration unit. The kit of the invention may also contain a first recipient comprising a dried/lyophilized antigen-binding molecule and a second recipient comprising an aqueous formulation. In certain embodiments of this invention, kits containing single-chambered and multi-chambered pre-filled syringes (e.g., liquid syringes and lyosyringes) are provided.

It is noted that as used herein, the singular forms “a”, “an”, and “the”, include plural references unless the context clearly indicates otherwise. Thus, for example, reference to “a reagent” includes one or more of such different reagents and reference to “the method” includes reference to equivalent steps and methods known to those of ordinary skill in the art that could be modified or substituted for the methods described herein.

Unless otherwise indicated, the term “at least” preceding a series of elements is to be understood to refer to every element in the series. Those skilled in the art will recognize, or be able to ascertain using no more than routine experimentation, many equivalents to the specific embodiments of the invention described herein. Such equivalents are intended to be encompassed by the present invention.

The term “and/or” wherever used herein includes the meaning of “and”, “or” and “all or any other combination of the elements connected by said term”.

The term “about” or “approximately” as used herein means within 20%, preferably within 10%, and more preferably within 5% of a given value or range. It includes, however, also the concrete number, e.g., about 20 includes 20.

The term “less than” or “greater than” includes the concrete number. For example, less than 20 means less than or equal to. Similarly, more than or greater than means more than or equal to, or greater than or equal to, respectively.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integer or step. When used herein the term “comprising” can be substituted with the term “containing” or “including” or sometimes when used herein with the term “having”.

When used herein “consisting of” excludes any element, step, or ingredient not specified in the claim element. When used herein, “consisting essentially of” does not exclude materials or steps that do not materially affect the basic and novel characteristics of the claim.

In each instance herein any of the terms “comprising”, “consisting essentially of” and “consisting of” may be replaced with either of the other two terms.

It should be understood that this invention is not limited to the particular methodology, protocols, material, reagents, and substances, etc., described herein and as such can vary. The terminology used herein is for the purpose of describing particular embodiments only, and is not intended to limit the scope of the present invention, which is defined solely by the claims.

All publications and patents cited throughout the text of this specification (including all patents, patent applications, scientific publications, manufacturer's specifications, instructions, etc.), whether supra or infra, are hereby incorporated by reference in their entirety. Nothing herein is to be construed as an admission that the invention is not entitled to antedate such disclosure by virtue of prior invention. To the extent the material incorporated by reference contradicts or is inconsistent with this specification, the specification will supersede any such material.

A better understanding of the present invention and of its advantages will be obtained from the following examples, offered for illustrative purposes only. The examples are not intended to limit the scope of the present invention in any way.

### EXAMPLES

### Example 1: Luciferase-Based Cytotoxicity Assay with Unstimulated Human PBMC on Multitargeting Bispecific Antigen-Binding Molecules to Determine Beneficial Efficacy Gap

### Isolation of Effector Cells

Human peripheral blood mononuclear cells (PBMC) were prepared by Ficoll density gradient centrifugation from enriched lymphocyte preparations (buffy coats), a side product of blood banks collecting blood for transfusions. Buffy coats were supplied by a local blood bank and PBMC were prepared on the day after blood collection. After Ficoll density centrifugation and extensive washes with Dulbecco's PBS (Gibco), remaining erythrocytes were removed from PBMC via incubation with erythrocyte lysis buffer (155 mM NH₄Cl, 10 mM KHCO₃, 100 μM EDTA). Remaining lymphocytes mainly encompass B and T lymphocytes, NK cells and monocytes. PBMC were kept in culture at 37° C./5% CO₂ in RPMI medium (Gibco) with 10% FCS (Gibco).

Depletion of CD14⁺ and CD56⁺ Cells

For depletion of CD14⁺ cells, human CD14 MicroBeads (Milteny Biotec, MACS, #130-050-201) were used, for depletion of NK cells human CD56 MicroBeads (MACS, #130-050-401). PBMC were counted and centrifuged for 10 min at room temperature with 300×g. The supernatant was discarded and the cell pellet resuspended in MACS isolation buffer (60 μL/10⁷ cells). CD14 MicroBeads and CD56 MicroBeads (20 μL/10⁷ cells) were added and incubated for 15 min at 4-8° C. The cells were washed with AutoMACS rinsing buffer (Milteny #130-091-222) (1-2 mL/10⁷ cells). After centrifugation (see above), supernatant was discarded and cells resuspended in MACS isolation buffer (500 μL/10⁸ cells). CD14/CD56 negative cells were then isolated using LS Columns (Milteny Biotec, #130-042-401). PBMC w/o CD14+/CD56+ cells were adjusted to 1.2×10⁶ cells/mL and cultured in RPMI complete medium i.e. RPMI1640 (Biochrom AG, #FG1215) supplemented with 10% FBS (Bio West, #S1810), 1× non-essential amino acids (Biochrom AG, #K0293), 10 mM Hepes buffer (Biochrom AG, #L1613), 1 mM sodium pyruvate (Biochrom AG, #L0473) and 100 U/mL penicillin/streptomycin (Biochrom AG, #A2213) at 37° C. in an incubator until needed.

### Target Cell Preparation

Cells were harvested, spinned down and adjusted to 1.2×10⁵ cells/mL in complete RPMI medium. The vitality of cells was determined using Nucleocounter NC-250 (Chemometec) and Solution18 Dye containing Acridine Orange and DAPI (Chemometec).

### Luciferase Based Analysis

This assay was designed to quantify the lysis of target cells in the presence of serial dilutions of multi-specific antigen-binding molecules. Equal volumes of Luciferase-positive target cells and effector cells (i.e., PBMC w/o CD14+; CD56+ cells) were mixed, resulting in an E:T cell ratio of 10:1. 42 μL of this suspension were transferred to each well of a 384-well plate. 8 μL of serial dilutions of the corresponding multi-specific antigen-binding molecules and a negative control antigen-binding molecules (a CD3-based antigen-binding molecule recognizing an irrelevant target antigen) or RPMI complete medium as an additional negative control were added. The multi-specific antibody-mediated cytotoxic reaction proceeded for 48 hours in a 5% CO₂ humidified incubator. Then 25 μL substrate (Steady-Glo® Reagent, Promega) were transferred to the 384-well plate. Only living, Luciferase-positive cells react to the substrate and thus create a luminescence signal. Samples were measured with a SPARK microplate reader (TECAN) and analyzed by Spark Control Magellan software (TECAN).

Percentage of cytotoxicity was calculated as follows:

$$Cytoxicity [ \% ] = ( 1 - \frac{{RLU}_{Sample}}{{RLU}_{Negative ‐ Control}} ) \times 100$$

$$RLU = relative light units$$

$$Negative ‐ Control = cells without multi ‐ specific antigen ‐ binding molecule$$

Using GraphPad Prism 7.04 software (Graph Pad Software, San Diego), the percentage of cytotoxicity was plotted against the corresponding multi-specific antigen-binding molecule concentrations. Dose response curves were analyzed with the four parametric logistic regression models for evaluation of sigmoid dose response curves with fixed hill slope and EC50 values were calculated.

The following mono and double target expressing cell lines were used for the Luciferase-based cytotoxicity assay:
GSU-LUC wt (CDH3+ and MSLN+)
GSU-LUC KO CDH3 (CDH3− and MSLN+)
GSU-LUC KO MSLN (CDH3+ and MSLN−)
HCT 116-LUC wt (CDH3+ and MSLN+)
HCT 116-LUC KO CDH3 (CDH3− and MSLN+)
HCT 116-LUC KO MSLN (CDH3+ and MSLN−)

TABLE 4
Overiew on MSLN-CDH3 T-cell engaging
cytotoxicity assays on 9 different test molecules
A) Effector cells: human unstimulated T cells
Target cells: GSU wt, GSU KO CDH3, GSU KO MSLN

| | EC50 |
|---|---|
| | [pM] |
| MSLN-CDH3 T-cell engager molecule 1 on GSU wt | 0.151 |
| MSLN-CDH3 T-cell engager molecule 1 on GSU KO CDH3 | 339 |
| MSLN-CDH3 T-cell engager molecule 1 on GSU KO MSLN | 256 |
| MSLN-CDH3 T-cell engager molecule 2 on GSU wt | 1.389 |
| MSLN-CDH3 T-cell engager molecule 2 on GSU KO CDH3 | 2900 |
| MSLN-CDH3 T-cell engager molecule 2 on GSU KO MSLN | 1725 |
| MSLN-CDH3 T-cell engager molecule 3 on GSU wt | 0.556 |
| MSLN-CDH3 T-cell engager molecule 3 on GSU KO CDH3 | 115 |
| MSLN-CDH3 T-cell engager molecule 3 on GSU KO MSLN | 502 |
| MSLN-CDH3 T-cell engager molecule 4 on GSU wt | 1.489 |
| MSLN-CDH3 T-cell engager molecule 4 on GSU KO CDH3 | 315 |
| MSLN-CDH3 T-cell engager molecule 4 on GSU KO MSLN | 7657 |
| MSLN-CDH3 T-cell engager molecule 5 on GSU wt | 0.920 |
| MSLN-CDH3 T-cell engager molecule 5 on GSU KO CDH3 | 171 |
| MSLN-CDH3 T-cell engager molecule 5 on GSU KO MSLN | 3328 |
| MSLN-CDH3 T-cell engager molecule 6 on GSU wt | 1.334 |
| MSLN-CDH3 T-cell engager molecule 6 on GSU KO CDH3 | 512 |
| MSLN-CDH3 T-cell engager molecule 6 on GSU KO MSLN | 3243 |
| MSLN-CDH3 T-cell engager molecule 7 on GSU wt | 0.042 |
| MSLN-CDH3 T-cell engager molecule 7 on GSU KO CDH3 | 69.9 |
| MSLN-CDH3 T-cell engager molecule 7 on GSU KO MSLN | 7.9 |
| MSLN-CDH3 T-cell engager molecule 8 on GSU wt | 0.865 |
| MSLN-CDH3 T-cell engager molecule 8 on GSU KO CDH3 | 91.5 |
| MSLN-CDH3 T-cell engager molecule 8 on GSU KO MSLN | 136 |
| MSLN-CDH3 T-cell engager molecule 9 on GSU wt | 0.575 |
| MSLN-CDH3 T-cell engager molecule 9 on GSU KO CDH3 | 156 |
| MSLN-CDH3 T-cell engager molecule 9 on GSU KO MSLN | 2626 |
| MSLN T-cell engager molecule on GSU wt | 857 |
| MSLN T-cell engager molecule on GSU KO CDH3 | 760 |
| MSLN T-cell engager molecule on GSU KO MSLN | 16251 |
| CDH3 T-cell engager molecule on GSU wt | 262 |
| CDH3 T-cell engager molecule on GSU KO CDH3 | 328 |
| CDH3 T-cell engager molecule on GSU KO MSLN | 164 |
| EGFRvIII T-cell engager molecule on GSU wt | n/a |
| EGFRvIII T-cell engager molecule on GSU KO CDH3 | n/a |
| EGFRvIII T-cell engager molecule on GSU KO MSLN | n/a |

MSLN-CDH3 T-cell engager molecule 1: MS 15-B12 CC × I2L × G4 × scFc × G4 × CH3 15-E11 CC × I2L
MSLN-CDH3 T-cell engager molecule 2: MS 15-B12 CC × I2L × (G4Q)3 × scFc × (G4Q)3 × CH3 15-E11 CC × I2L
MSLN-CDH3 T-cell engager molecule 3: MS 15-B12 CC × I2L × G4 × scFc × G4 × CH3 15-E11 CC × I2L_GQ
MSLN-CDH3 T-cell engager molecule 4: CH3 15-E11 CC × I2L × (G4S)3 × scFc × (G4S)3 × MS 15-B12 CC × I2L
MSLN-CDH3 T-cell engager molecule 5: CH3 15-E11 CC × I2L × (G4Q)3 × scFc × (G4Q)3 × MS 15-B12 CC × I2L
MSLN-CDH3 T-cell engager molecule 6: CH3 15-E11 CC × I2L × G4 × scFc × G4 × MS 15-B12 CC × I2L_GQ
MSLN-CDH3 T-cell engager molecule 7: MS 15-B12 CC × I2M2 × (G4S)3 × scFc × (G4S)3 × CH3 15-E11 CC × I2M2
MSLN-CDH3 T-cell engager molecule 8: CH3 15-E11 CC × I2M2 × (G4S)3 × scFc × (G4S)3 × MS 15-B12 CC × I2M2
MSLN-CDH3 T-cell engager molecule 9: MS 15-B12 CC × I2M2 × G4 × scFc × G4 × CH3 005-D5 CC × I2M2
MSLN T-cell engager molecule (MSLN only binding): MS 5-F11 × I2C0 × scFc
CDH3 T-cell engager molecule (CDH3 only binding): CH3 G8A 6-B12 × I2C0 × scFc
EGFRvIII T-cell engager molecule (non-binding): EGFRvIII CC × I2C0 × scFc

### Detailed Results Indicating Efficacy Gaps:

TABLE 5
EC50 values in pM and gaps of naïve GSU cells versus knock-out GSU cells

| | EC50 GSU | | | | EC50 GSU |
|---|---|---|---|---|---|
| | KO MSLN | fold | EC50 GSU | fold | KO CDH3 |
| | [pM] | gap | wt [pM] | gap | [pM] |
| MSLN-CDH3 T-cell | 256 | 1695 | 0.151 | 2245 | 339 |
| engager molecule 1 | | | | | |
| MSLN-CDH3 T-cell | 1725 | 1242 | 1.389 | 2088 | 2900 |
| engager molecule 2 | | | | | |
| MSLN-CDH3 T-cell | 502 | 903 | 0.556 | 207 | 115 |
| engager molecule 3 | | | | | |
| MSLN-CDH3 T-cell | 7657 | 5142 | 1.489 | 212 | 315 |
| engager molecule 4 | | | | | |
| MSLN-CDH3 T-cell | 3328 | 3617 | 0.92 | 186 | 171 |
| engager molecule 5 | | | | | |
| MSLN-CDH3 T-cell | 3243 | 2429 | 1.335 | 384 | 512 |
| engager molecule 6 | | | | | |
| MSLN-CDH3 T-cell | 7.9 | 187 | 0.042 | 1664 | 69.9 |
| engager molecule 7 | | | | | |
| MSLN-CDH3 T-cell | 136 | 157 | 0.865 | 106 | 91.5 |
| engager molecule 8 | | | | | |
| MSLN-CDH3 T-cell | 2626 | 4567 | 0.575 | 271 | 156 |
| engager molecule 9 | | | | | |
| MSLN T-cell | 16251 | 19.0 | 857 | 0.9 | 760 |
| engager molecule | | | | | |
| CDH3 T-cell | 164 | 0.6 | 262 | 1.3 | 328 |
| engager molecule | | | | | |
| EGFRvIII T-cell | n/a | n/a | n/a | n/a | n/a |
| engager molecule | | | | | |

The tested MSLN-CDH3 T-cell engager molecules 1-9 showed increased activity (lower EC50 values) on MSLN and CDH3 double positive GSU wt cells compared to respective GSU k.o cells (GSU CDH3 k.o and GSU MSLN k.o.). The MSLN-CDH3 T-cell engager molecules 1-9 showed EC50 gaps greater 100-fold on MSLN and CDH3 double positive GSU wt cells versus the respective GSU k.o cells (GSU CDH3 k.o and GSU MSLN k.o.) (Fig. A) and Table 5).

TABLE 6
Overview on the efficacy of 9 tested
molecules using the following cell lines:
Effector cells: human unstimulated T cells
Target cells: HCT 116 wt, HCT 116 KO CDH3, HCT 116 KO MSLN

| | EC50 |
|---|---|
| | [pM] |
| MSLN-CDH3 T-cell engager molecule 1 on HCT 116 wt | 0.0076 |
| MSLN-CDH3 T-cell engager molecule 1 on HCT 116 KO CDH3 | 18.3 |
| MSLN-CDH3 T-cell engager molecule 1 on HCT 116 KO MSLN | 1.1 |
| MSLN-CDH3 T-cell engager molecule 2 on HCT 116 wt | 0.0261 |
| MSLN-CDH3 T-cell engager molecule 2 on HCT 116 KO CDH3 | 29.2 |
| MSLN-CDH3 T-cell engager molecule 2 on HCT 116 KO MSLN | 3.5 |
| MSLN-CDH3 T-cell engager molecule 3 on HCT 116 wt | 0.0060 |
| MSLN-CDH3 T-cell engager molecule 3 on HCT 116 KO CDH3 | 44.5 |
| MSLN-CDH3 T-cell engager molecule 3 on HCT 116 KO MSLN | 1.0 |
| MSLN-CDH3 T-cell engager molecule 4 on HCT 116 wt | 0.0481 |
| MSLN-CDH3 T-cell engager molecule 4 on HCT 116 KO CDH3 | 37.8 |
| MSLN-CDH3 T-cell engager molecule 4 on HCT 116 KO MSLN | 24.4 |
| MSLN-CDH3 T-cell engager molecule 5 on HCT 116 wt | 0.0283 |
| MSLN-CDH3 T-cell engager molecule 5 on HCT 116 KO CDH3 | 22.6 |
| MSLN-CDH3 T-cell engager molecule 5 on HCT 116 KO MSLN | 8.5 |
| MSLN-CDH3 T-cell engager molecule 6 on HCT 116 wt | 0.0713 |
| MSLN-CDH3 T-cell engager molecule 6 on HCT 116 KO CDH3 | 51.8 |
| MSLN-CDH3 T-cell engager molecule 6 on HCT 116 KO MSLN | 8.7 |
| MSLN-CDH3 T-cell engager molecule 7 on HCT 116 wt | 0.0002 |
| MSLN-CDH3 T-cell engager molecule 7 on HCT 116 KO CDH3 | 0.69 |
| MSLN-CDH3 T-cell engager molecule 7 on HCT 116 KO MSLN | 0.20 |
| MSLN-CDH3 T-cell engager molecule 8 on HCT 116 wt | 0.0007 |
| MSLN-CDH3 T-cell engager molecule 8 on HCT 116 KO CDH3 | 1.1 |
| MSLN-CDH3 T-cell engager molecule 8 on HCT 116 KO MSLN | 0.24 |
| MSLN-CDH3 T-cell engager molecule 9 on HCT 116 wt | 0.0166 |
| MSLN-CDH3 T-cell engager molecule 9 on HCT 116 KO CDH3 | 4.4 |
| MSLN-CDH3 T-cell engager molecule 9 on HCT 116 KO MSLN | 4.1 |
| MSLN T-cell engager molecule on HCT 116 wt | 0.3 |
| MSLN T-cell engager molecule on HCT 116 KO CDH3 | 0.69 |
| MSLN T-cell engager molecule on HCT 116 KO MSLN | 98.9 |
| CDH3 T-cell engager molecule on HCT 116 wt | 1.6 |
| CDH3 T-cell engager molecule on HCT 116 KO CDH3 | 2055 |
| CDH3 T-cell engager molecule on HCT 116 KO MSLN | 1.4 |
| EGFRvIII T-cell engager molecule on HCT 116 wt | 8016 |
| EGFRvIII T-cell engager molecule on HCT 116 KO CDH3 | 7127 |
| EGFRvIII T-cell engager molecule on HCT 116 KO MSLN | 11184 |

MSLN-CDH3 T-cell engager molecule 1: MS 15-B12 CC × I2L × G4 × scFc × G4 × CH3 15-E11 CC × I2L
MSLN-CDH3 T-cell engager molecule 2: MS 15-B12 CC × I2L × (G4Q)3 × scFc × (G4Q)3 × CH3 15-E11 CC × I2L
MSLN-CDH3 T-cell engager molecule 3: MS 15-B12 CC × I2L × G4 × scFc × G4 × CH3 15-E11 CC × I2L_GQ
MSLN-CDH3 T-cell engager molecule 4: CH3 15-E11 CC × I2L × (G4S)3 × scFc × (G4S)3 × MS 15-B12 CC × I2L
MSLN-CDH3 T-cell engager molecule 5: CH3 15-E11 CC × I2L × (G4Q)3 × scFc × (G4Q)3 × MS 15-B12 CC × I2L
MSLN-CDH3 T-cell engager molecule 6: CH3 15-E11 CC × I2L × G4 × scFc × G4 × MS 15-B12 CC × I2L_GQ
MSLN-CDH3 T-cell engager molecule 7: MS 15-B12 CC × I2M2 × (G4S)3 × scFc × (G4S)3 × CH3 15-E11 CC × I2M2
MSLN-CDH3 T-cell engager molecule 8: CH3 15-E11 CC × I2M2 × (G4S)3 × scFc × (G4S)3 × MS 15-B12 CC × I2M2
MSLN-CDH3 T-cell engager molecule 9: MS 15-B12 CC × I2M2 × G4 × scFc × G4 × CH3 005-D5 CC × I2M2
MSLN T-cell engager molecule (MSLN only binding): MS 5-F11 × I2C0 × scFc
CDH3 T-cell engager molecule (CDH3 only binding): CH3 G8A 6-B12 × I2C0 × scFc
EGFRvIII T-cell engager molecule (non-binding): EGFRvIII CC × I2C0 × scFc

### Results:

TABLE 7
EC50 values in pM and gaps of naïve
HCT 116 cells versus knock-out HCT 116 cells

| | EC50 HCT | | EC50 HCT | | EC50 HCT |
|---|---|---|---|---|---|
| | 116 KO | fold | 116 wt | fold | 116 KO |
| | MSLN [pM] | gap | [pM] | gap | CDH3 [pM] |
| MSLN-CDH3 T-cell | 1.1 | 145 | 0.0076 | 2408 | 18.3 |
| engager molecule 1 | | | | | |
| MSLN-CDH3 T-cell | 3.5 | 134 | 0.0261 | 1119 | 29.2 |
| engager molecule 2 | | | | | |
| MSLN-CDH3 T-cell | 1.0 | 167 | 0.006 | 7417 | 44.5 |
| engager molecule 3 | | | | | |
| MSLN-CDH3 T-cell | 24.4 | 507 | 0.0481 | 786 | 37.8 |
| engager molecule 4 | | | | | |
| MSLN-CDH3 T-cell | 8.5 | 300 | 0.0283 | 799 | 22.6 |
| engager molecule 5 | | | | | |
| MSLN-CDH3 T-cell | 8.7 | 122 | 0.0713 | 727 | 51.8 |
| engager molecule 6 | | | | | |
| MSLN-CDH3 T-cell | 0.02 | 100 | 0.0002 | 3450 | 0.69 |
| engager molecule 7 | | | | | |
| MSLN-CDH3 T-cell | 0.2 | 286 | 0.0007 | 1571 | 1.1 |
| engager molecule 8 | | | | | |
| MSLN-CDH3 T-cell | 4.1 | 247 | 0.0166 | 265 | 4.4 |
| engager molecule 9 | | | | | |
| MSLN T-cell | 98.9 | 330 | 0.3 | 2 | 0.69 |
| engager molecule | | | | | |
| CDH3 T-cell | 1.4 | 1 | 1.6 | 1289 | 2055 |
| engager molecule | | | | | |
| EGFRvIII T-cell | 11184 | n/a | 8016 | n/a | 7127 |
| engager molecule | | | | | |

The tested MSLN-CDH3 T-cell engager molecules 1-9 showed increased activity (lower EC50 values) on MSLN and CDH3 double positive HCT 116 wt cells compared to respective HCT 116 k.o cells (HCT 116 CDH3 k.o and HCT 116 MSLN k.o.). The MSLN-CDH3 T-cell engager molecules 1-9 showed EC50 gaps greater 100-fold on MSLN and CDH3 double positive HCT 116 wt cells versus the respective HCT 116 k.o cells (HCT 116 CDH3 k.o and HCT 116 MSLN k.o.) (Fig. B) and Table 7).

TABLE 8
Overview on the efficacy of molecule 6 using the following cell lines:
Effector cells: human unstimulated T cells
Target cells: GSU wt, GSU KO CDH3, GSU KO MSLN
Test molecule: MSLN-CDH3 T-cell engager molecule 6

| | EC50 [pM] |
|---|---|
| E:T ratio 10:1 on GSU wt | 0.6 |
| E:T ratio 10:1 on GSU KO CDH3 | 39.6 |
| E:T ratio 10:1 on GSU KO MSLN | 98.0 |
| E:T ratio 1:1 on GSU wt | 0.8 |
| E:T ratio 1:1 on GSU KO CDH3 | 108 |
| E:T ratio 1:1 on GSU KO MSLN | 322 |
| E:T ratio 1:2 on GSU wt | 1.0 |
| E:T ratio 1:2 on GSU KO CDH3 | 446 |
| E:T ratio 1:2 on GSU KO MSLN | 341 |

Legend:
MSLN-CDH3 T-cell engager molecule 6: CH3 15-E11 CC × I2L × G4 × scFc × G4 × MS 15-B12 CC × I2L_GQ

### Results:

TABLE 9
MSLN-CDH3 T-cell engager molecule 6 EC50 values
and gaps of naïve GSU cells versus GSU knock-out
cells using different effector:target ratios

| | EC50 | | EC50 | | EC50 |
|---|---|---|---|---|---|
| | GSU KO | fold | GSU | fold | GSU KO |
| E:T Ratio | MSLN [pM] | gap | wt [pM] | gap | CDH3 [pM] |
| 10:1 | 98.0 | 163 | 0.6 | 66 | 39.5 |
| 1:1 | 322 | 403 | 0.8 | 135 | 108 |
| 1:2 | 341 | 341 | 1.0 | 446 | 446 |

The MSLN-CDH3 T-cell engager molecule 6 showed EC50 gaps greater 100-fold on MSLN and CDH3 double positive GSU wt cells versus the respective GSU k.o cells (GSU CDH3 k.o and GSU MSLN k.o.) at different E:T ratios of 10:1, 1:2 and 1:1. At lower E:T ratios such as 1:2 and 1:1 greater EC50 gaps were achieved compared to the gaps observed at a higher E:T ratio of 10:1 (FIG. C) and Table 9).

### Example 2: Selectivity Gap of Multitargeting Antigen-Binding Molecules of the Invention

FACS-Based Cytotoxicity Assay with Unstimulated Human PBMC

### Isolation of Effector Cells

Human peripheral blood mononuclear cells (PBMC) were prepared by Ficoll density gradient centrifugation from enriched lymphocyte preparations (buffy coats), a side product of blood banks collecting blood for transfusions. Buffy coats were supplied by a local blood bank and PBMC were prepared on the day after blood collection. After Ficoll density centrifugation and extensive washes with Dulbecco's PBS (Gibco), remaining erythrocytes were removed from PBMC via incubation with erythrocyte lysis buffer (155 mM NH4Cl, 10 mM KHCO3, 100 μM EDTA). Remaining lymphocytes mainly encompass B and T lymphocytes, NK cells and monocytes. PBMC were kept in culture at 37° C./5% CO2 in RPMI medium (Gibco) with 10% FBS (Bio West, #S1810).

### Isolation of Human T-Cells

For isolation of human T-cells, Pan T Cell Isolation Kit, human (Miltenyi Biotec, MACS, #130-096-535) was used to deplete non-target cells, i.e., monocytes, neutrophils, eosinophils, B cells, stem cells, dendritic cells, NK cells, granulocytes, or erythroid cells from the PBMC cell solution. Therefore, respective number of PBMC was centrifuged for 10 min at room temperature at 300×g. Supernatant was discarded, and the cell pellet was resuspended in MACS isolation buffer (Dulbecco's PBS (Gibco), 100 μM EDTA, 0.5% FBS (Bio West. #S1810)) [40 μl buffer/1×107 cells]. Pan T Cell Biotin-Antibody cocktail [10 μL/1×107 cells] was added and suspension was incubated for 5 min at 4° C. Afterwards, MACS isolation buffer was added [30 μl buffer/1×107 cells] together with Anti-Biotin MicroBeads [20 μl/1×107 cells)] and cell suspension was left at 4° C. for 10 min. The cell solution was then applied to LS Columns (Miltenyi Biotec, #130-042-401) in the magnetic field of a suitable Miltenyi Separator to isolate untouched T cells while magnetically labelled non-T-cells remain on the column. Columns were washed 3 times with MACS isolation buffer. Column flowthrough was centrifuged (see above), supernatant was discarded and cells were resuspended in RPMI complete medium i.e. RPMI1640 (Biochrom AG, #FG1215) supplemented with 10% FBS (Bio West, #S1810), 1× non-essential amino acids (Biochrom AG, #K0293), 1 mM sodium pyruvate (Biochrom AG, #L0473) and 100 U/mL penicillin/streptomycin (Biochrom AG, #A2213) and incubated at 37° C. until needed.

### Target Cell Labeling for Flow-Cytometry Based T-Cell-Dependent Cellular Cytotoxicity (TDCC) Assay

For the analysis of cell lysis in flow cytometry assays, the fluorescent membrane dye DiOC18 (DiO) (Thermo Fisher, #V22886) was used to label human-target transfected CHO cells or cancer cell lines as target cells and distinguish them from effector cells. Briefly, cells were harvested, washed once with PBS and adjusted to 106 cell/mL in PBS containing the membrane dye DiO (5 μL/106 cells). After incubation for 3 min at 37° C., cells were washed twice in complete RPMI medium and directly used in assay.

### Setup of Flow Cytometry-Based T-Cell-Dependent Cellular Cytotoxicity (TDCC) Assay and Analysis

Cytotoxic activity of bispecific T-cell engager molecules was determined through the capability of inducing T-cell mediated target cell lysis. Therefore, the lysis of human target cells in the presence of serial dilutions of bispecific T-cell engager molecules and effector cells was analyzed.

DiO-labeled target-cells and effector cells (i.e., Pan T-cells) were mixed at an effector to target-cell (E:T) ratio of 10:1 and incubated with serial dilutions of the corresponding bispecific T-cell engager molecule in 96-well plates. Plates were incubated at 37° C., 5% CO2 and 95% relative humidity for 48 h. On day of assay analysis, cells were transferred to a new 96-well plate and loss of target cell membrane integrity was monitored by adding propidium iodide (PI) at a final concentration of 1 μg/mL. PI is a membrane impermeable dye that normally is excluded from viable cells, whereas dead cells take it up and become identifiable by fluorescent emission.

Samples were measured by flow cytometry on an iQue Plus (Intellicyt, now Sartorius) instrument and analyzed by Forecyt software (Intellicyt). Target cells were identified as DiO-positive cells. PI-negative target cells were classified as living target cells. Percentage of specific cell lysis respective cytotoxicity was calculated according to the following formula:

$$Cytoxicity [ \% ] = ( \frac{{n}_{dead target cells}}{{n}_{target cells}} ) \times 100$$

n=number of events per well

In some experiments, the cytotoxicity was calculated according to this formula:

$$Cytotoxicity [ \% ] = ( 1 - \frac{{\%}_{viable target cells molecule ‐ treated}}{{\%}_{viable target cells untreated}} ) \times 100$$

n=number of events per well

Using GraphPad Prism 7.04 software (Graph Pad Software, San Diego), the percentage of cytotoxicity was plotted against the corresponding bispecific T-cell engager molecule concentrations. Sigmoidal dose response curves were analyzed with the four parametric logistic regression models with variable slope and EC50 values were calculated.

The following target cell lines were used for the FACS-based cytotoxicity assay:

CHO huMSLN:

Parental CHO (DHFR−) cells transfected with human MSLN on pEFDHFR-MTX1 for expression of human MSLN and dummy sequence on pEFDHFR-MTX2

CHO huEpCAM:

Parental CHO (DHFR−) cells transfected with human EpCAM on pEFDHFR-MTX2 for expression of human EpCAM and dummy sequence on pEFDHFR-MTX1

CHO huMSLN huEpCAM:

Parental CHO (DHFR−) cells transfected with human MSLN on pEFDHFR-MTX1 and human EpCAM on pEFDHFR-MTX2 for simultaneous expression of human MSLN and human EpCAM

CHO huCLL1:

Parental CHO (DHFR−) cells transfected with human CLL1 on pEFDHFR for expression of human CLL1

CHO huFLT3:

Parental CHO (DHFR−) cells transfected with human FLT3 on pEFDHFR for expression of human FLT3

CHO huCLL1 huFLT3:

Parental CHO (DHFR−) cells transfected with human CLL1 on pEFDHFR-MTX1 and human FLT3 on pEFDHFR-MTX2 for simultaneous expression of human CLL1 and human FLT3

### SW48 WT:

Parental cell line, wildtype (WT)

### SW48 MSLN KO:

Parental cell line SW48, in which MSLN gene was knocked out (KO)

### SW48 CDH3 KO:

Parental cell line SW48, in which CDH3 gene was knocked out (KO)

### Cytokine Measurement of In Vitro TDCC Assay

Cytokine release during TDCC in-vitro assay was measured with BD™ Cytometric Bead Array Human Th1/Th2 Cytokine Kit II (BD Biosciences, #551809). Therefore, two cytotoxicity assay sets were set up with full PBMC as effector cells. After 24 h, the supernatant of one assay plate set was removed and analyzed for the levels of human cytokines IL-2, IL-4, IL-6, IL-10, TNFα und IFNγ according to the manufacturer's protocol. After 48 h, the cytotoxic activity of the other assay set was measured.

### Setup of Luciferase-Based T-Cell-Dependent Cellular Cytotoxicity (TDCC) Assay and Analysis

Luc-positive target-cells and effector cells (i.e., Pan T-cells) were mixed at an effector to target-cell (E:T) ratio of 10:1 and incubated with serial dilutions of the corresponding bispecific T-cell engager molecule in 384-well plates. The multitargeting antibody-mediated cytotoxic reaction proceeded for 48 hours in a 5% CO2 humidified incubator. Then 25 L substrate (Steady-Glo® Reagent, Promega) were transferred to the 384-well plate. Only living, luciferase-positive cells react to the substrate and thus create a luminescence signal. Samples were measured with a SPARK microplate reader (TECAN) and analyzed by Spark Control Magellan software (TECAN).

Percentage of cytotoxicity was calculated as follows:

$$Cytoxicity [ \% ] = ( 1 - \frac{{RLU}_{Sample}}{{RLU}_{Negative Control}} ) \times 100$$

RLU=relative light units

Negative-Control=cells without multi-specific antigen-binding molecule

Using GraphPad Prism 7.04 software (Graph Pad Software, San Diego), the percentage of cytotoxicity was plotted against the corresponding bispecific T-cell engager molecule concentrations. Sigmoidal dose response curves were analyzed with the four parametric logistic regression models with variable slope and EC50 values were calculated. Following target cell lines were used for the Luciferase-based cytotoxicity assay:

### HCT 116 LUC WT:

Parental cell line, wildtype (WT), transfected with luciferase

### HCT 116 LUC MSLN KO:

Parental cell line HCT 116 LUC, in which MSLN gene was knocked out (KO)

### HCT 116 LUC CDH3 KO:

Parental cell line HCT 116 LUC, in which CDH3 gene was knocked out (KO)

TABLE 10
EC50 values of mono versus dual targeting molecules on double
positive CHO cells versus single positive CHO cells;

| | | EC50 |
|---|---|---|
| | | [pM] |
| CLL1-FLT3 T-cell engager molecule 1 on | CHO huCLL1 | b.c.t |
| CLL1-FLT3 T-cell engager molecule 1 on | CHO huFLT3 | b.c.t |
| CLL1-FLT3 T-cell engager molecule 1 on | CHO huCLL1 huFLT3 | 1.9 |
| FLT3 T-cell engager molecule on | CHO huCLL1 | b.c.t. |
| FLT3 T-cell engager molecule on | CHO huFLT3 | 149 |
| FLT3 T-cell engager molecule on | CHO huCLL1 huFLT3 | 57 |
| CLL1 T-cell engager molecule on | CHO huCLL1 | 76 |
| CLL1 T-cell engager molecule on | CHO huFLT3 | b.c.t. |
| CLL1 T-cell engager molecule on | CHO huCLL1 huFLT3 | 36 |

b.c.t: below calculation threshold

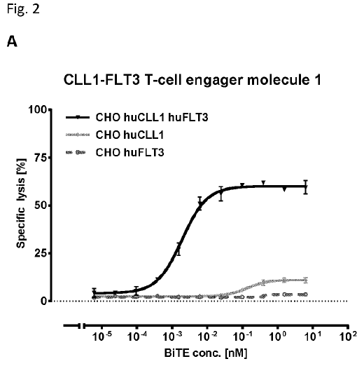

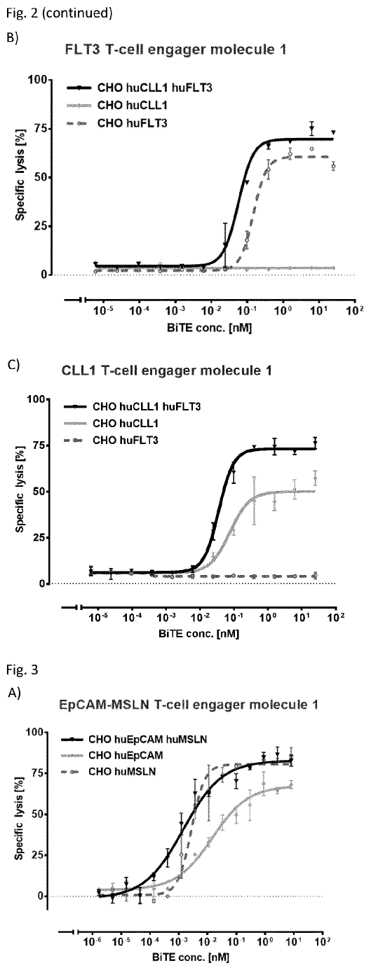

FIG. 2 shows cytotoxicity curves and EC50 values of CLL1-FLT3 T-cell engager molecules and mono targeting control T-cell engager molecules on double positive CHO huCLL1 huFLT3 target cells and single positive CHO huCLL1 or CHO huFLT3 target cells. Effector cells were unstimulated Pan T-cells. b.c.t: below calculation threshold

TABLE 11
EC50 values and selectivity gaps of double positive
CHO cells versus single positive CHO cells;

| | CHO | Gap double | CHO | Gap double | CHO |
|---|---|---|---|---|---|
| | huCLL1 | positive | huCLL1 | positive | huFLT3 |
| | EC50 | to single | huFLT3 | to single | EC50 |
| | [pM] | positive | EC50 [pM] | positive | [pM] |
| CLL1-FLT3 T-cell | b.c.t | >1000 | 1.9 | >1000 | b.c.t |
| engager molecule 1 | | | | | |
| CLL1 T-cell | 76 | 2 | 35.6 | — | b.c.t |
| engager molecule 1 | | | | | |
| FLT3 T-cell | b.c.t | — | 56.9 | 3 | 149 |
| engager molecule 1 | | | | | |

Legend

| | |
|---|---|
| CLL1-FLT3 T-cell | CL1 9-G4 CC × I2C cc × scFc × FL 4-E9 |
| engager molecule 1 | CC × I2C cc |
| CLL1 T-cell | CL1 9-G4 CC × PSMA 76B10 × I2C0 × |
| engager molecule 1 | scFc |
| FLT3 T-cell | PSMA 76-B10 × FL 4-E9 CC × I2C0 × scFc |
| engager molecule 1 | |

b.c.t: below calculation threshold

Results: CLL-FLT3 T-cell engager molecule 1 showed an increased activity (lower EC50 value) on huCLL1 and huFLT3 double positive target cells compared to huCLL1 or huFLT3 single positive target cells. This molecule showed EC50 selectivity gaps greater 1000-fold on double positive target cells versus single positive target cells. CLL-FLT3 T-cell engager molecule 1 contains two I2C binding domains with a disulfide bridge facilitated by two cysteine substitutions in the scFv framework at position 44 and 100 after Kabat numbering (further called I2C cc 44/100 or I2C cc). Mono targeting control T-cell engager molecules had comparable activity on single positive vs. double positive cells (difference only 2-3 fold).

### Example 3: Selectivity Gap of Different Multitargeting Bispecific T-Cell Engager Polypeptide (MBiTEP) Formats

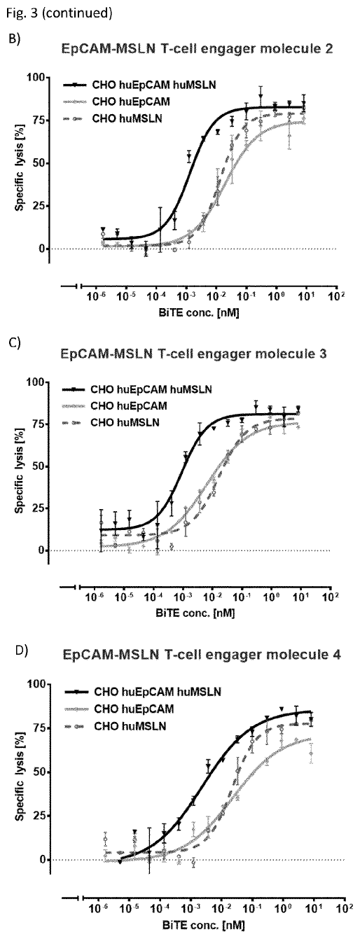

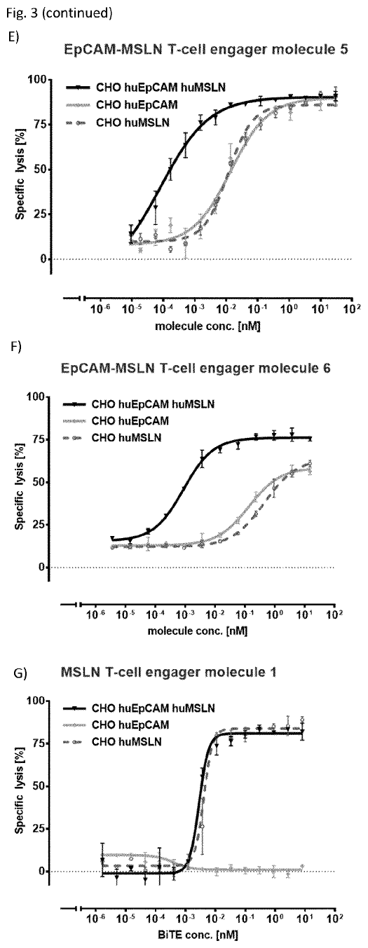

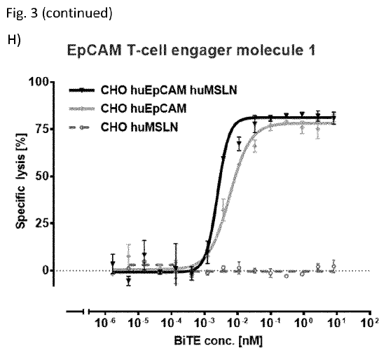

FIG. 3: Cytotoxicity curves of EpCAM MSLN T-cell engager molecules and mono targeting control T-cell engager molecules on double positive CHO huEpCAM huMSLN target cells and single positive CHO huEpCAM or CHO huMSLN target cells. Effector cells were unstimulated Pan T-cells.

TABLE 12
EC50 values and selectivity gaps of double positive
CHO cells versus single positive CHO cells;

| | | Gap | | Gap | |
|---|---|---|---|---|---|
| | | double | CHO | double | CHO |
| | CHO | positive to | huEpCAM | positive to | huMSLN |
| | huEpCAM | single | huMSLN | single | EC50 |
| | EC50 [pM] | positive | EC50 [pM] | positive | [pM] |
| EpCAM-MSLN T-cell | 15 | 11 | 1.4 | 2 | 2.7 |
| engager molecule 1 | | | | | |
| EpCAM-MSLN T-cell | 19 | 15 | 1.3 | 10 | 13 |
| engager molecule 2 | | | | | |
| EpCAM-MSLN T-cell | 6.4 | 7 | 0.9 | 17 | 16 |
| engager molecule 3 | | | | | |
| EpCAM-MSLN T-cell | 23 | 10 | 2.3 | 10 | 22 |
| engager molecule 4 | | | | | |
| EpCAM-MSLN T-cell | 14 | 176 | 0.1 | 151 | 12 |
| engager molecule 5 | | | | | |
| EpCAM-MSLN T-cell | 135 | 155 | 0.9 | 511 | 445 |
| engager molecule 6 | | | | | |
| MSLN T-cell | 5.7 | 2 | 3.5 | — | b.c.t. |
| engager molecule 1 | | | | | |
| EpCAM T-cell | b.c.t. | — | 2.8 | 1 | 4.1 |
| engager molecule 1 | | | | | |

Legend

| | |
|---|---|
| EpCAM-MSLN T-cell | EpCAM 5-10 × H2 × scFc × I2Ccc × |
| engager molecule 1 | I2Ccc |
| EpCAM-MSLN T-cell | EpCAM 5-10 × H2 × I2Ccc × I2Ccc × |
| engager molecule 2 | scFc |
| EpCAM-MSLN T-cell | EpCAM 5-10 × H2 × I2Ccc × scFc × |
| engager molecule 3 | I2Ccc |
| EpCAM-MSLN T-cell | EpCAM 5-10 × scFc × H2 × I2Ccc × |
| engager molecule 4 | I2Ccc |
| EpCAM-MSLN T-cell | EpCAM 5-10 × I2Ccc × scFc × I2Ccc × |
| engager molecule 5 | H2 |
| EpCAM-MSLN T-cell | EpCAM 5-10 × I2Ccc × scFc × H2 × |
| engager molecule 6 | I2Ccc0 |
| EpCAM T-cell | EpCAM 5-10 × I2C × scFc |
| engager molecule 1 | |
| MSLN T-cell | MSLN 5F11 × I2C × scFc |
| engager molecule 1 | |

b.c.t: below calculation threshold

Results: From the tested EpCAM MSLN T-cell engager molecule 1-6, EpCAM MSLN T-cell engager molecule 5 and 6 show a selectivity gap between double positive and single positive target cells >100-fold. EpCAM MSLN T-cell engager molecule 5 and 6 have one bispecific entity (target binding domain and CD3 binding domain) at the N-terminus and one bispecific entity at the C-terminus, separated by a single chain Fc domain [target binding domain×CD3 binding domain×scFc×CD3 binding domain×target binding domain in EpCAM MSLN T-cell engager molecule 5 respectively target binding domain×CD3 binding domain×scFc×target binding domain×CD3 binding domain in EpCAM MSLN T-cell engager molecule 6]. Mono targeting control T-cell engager molecules had comparable activity on single positive vs. double positive cells (difference only 1-2 fold).

### Example 4 Selectivity Gap of Multitargeting Bispecific T-Cell Engager Polypeptides with Different Linker Between Target Binding Domain and CD3 Binding Domain

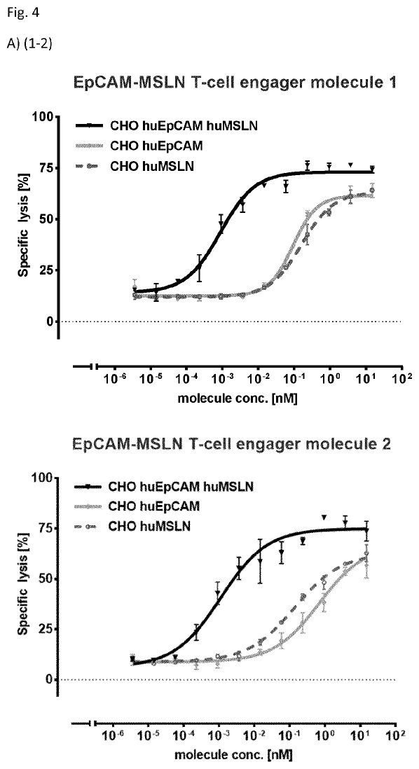

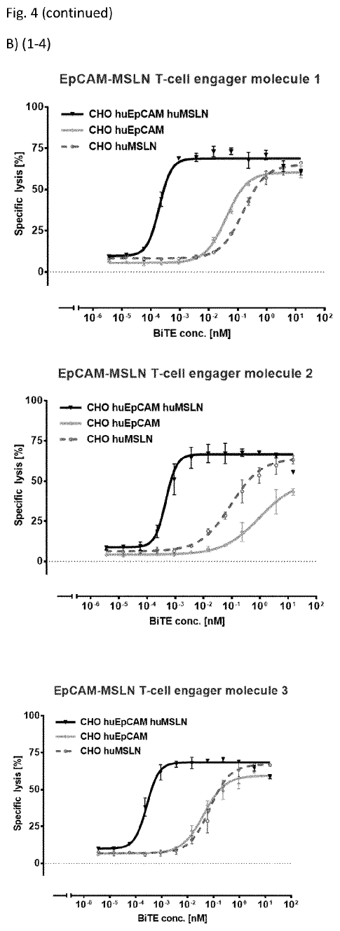

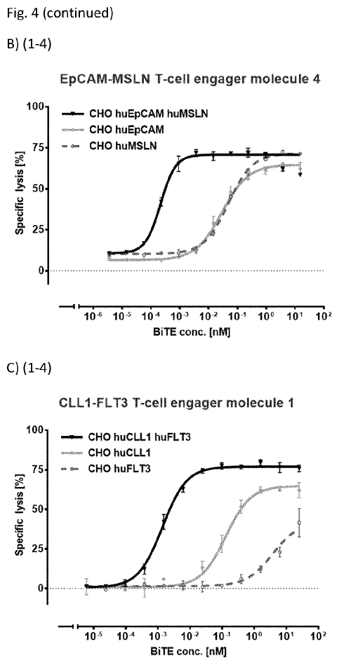

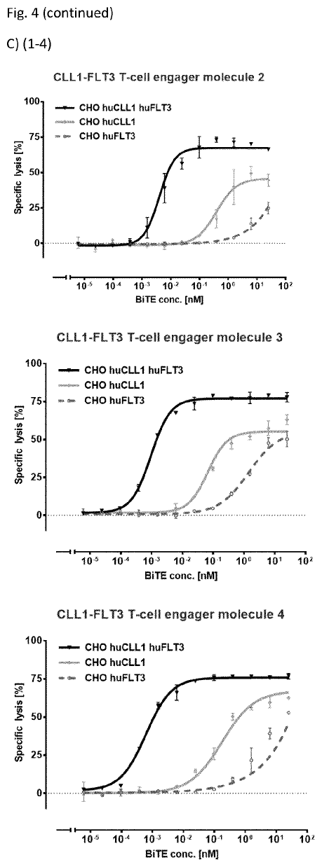

FIG. 4A: Cytotoxicity curves of EpCAM MSLN T-cell engager molecules on double positive CHO huEpCAM huMSLN target cells and single positive CHO huEpCAM or CHO huMSLN target cells. Effector cells were unstimulated Pan T-cells.

TABLE 13
EC50 values and selectivity gaps of double positive
CHO cells versus single positive CHO cells

| | CHO | Gap double | CHO | Gap double | CHO |
|---|---|---|---|---|---|
| | huEpCAM | positive to | huEpCAM | positive to | huMSLN |
| | EC50 | single | huMSLN | single | EC50 |
| | [pM] | positive | EC50 [pM] | positive | [pM] |
| EpCAM-MSLN T-cell | 93 | 105 | 0.9 | 183 | 162 |
| engager molecule 1 | | | | | |
| EpCAM-MSLN T-cell | 446 | 541 | 0.8 | 175 | 145 |
| engager molecule 2 | | | | | |

Legend

| | |
|---|---|
| EpCAM-MSLN T-cell | EpCAM 5-10 × I2Ccc -scFc × I2Ccc × H2 |
| engager molecule 1 | |
| EpCAM-MSLN T-cell | EpCAM 5-10 × (G₄S)₁₀ × I2Ccc × scFc × |
| engager molecule 2 | I2Ccc × (G₄S)₁₀ × MSLN H2 |

Results: EpCAM-MSLN T-cell engager molecule 1 and 2 showed comparable activity on double positive CHO huEpCAM and huMSLN target cells. These molecules showed an increased activity (lower EC50 value) on double positive target cells compared to CHO huEpCAM or CHO huMSLN single positive target cells. EpCAM-MSLN T-cell engager 1 and 2 contain the same target binding and CD3 binding domains in the same orientation [target binding domain×CD3 binding domain×scFc×CD3 binding domain×target binding domain], but they differ in the linker sequences between target binding and CD3 binding domain. With both linker variants, the EC50 selectivity gap between double positive target cells versus single positive target is greater than 100-fold.

FIG. 4B: Cytotoxicity curves of EpCAM MSLN T-cell engager molecules on double positive CHO huEpCAM huMSLN target cells and single positive CHO huEpCAM or CHO huMSLN target cells. Effector cells were unstimulated Pan T-cells.

TABLE 14
EC50 values and selectivity gaps of double positive
CHO cells versus single positive CHO cells;

| | CHO | Gap double | CHO | Gap double | CHO |
|---|---|---|---|---|---|
| | huEpCAM | positive | huEpCAM | positive | huMSLN |
| | EC50 | to single | huMSLN | to single | EC50 |
| | [pM] | positive | EC50 [pM] | positive | [pM] |
| EpCAM-MSLN T-cell | 40 | 206 | 0.19 | 794 | 154 |
| engager molecule 1 | | | | | |
| EpCAM-MSLN T-cell | 978 | 1903 | 0.51 | >1000 | b.c.t. |
| engager molecule 2 | | | | | |
| EpCAM-MSLN T-cell | 34 | 148 | 0.23 | 383 | 89 |
| engager molecule 3 | | | | | |
| EpCAM-MSLN T-cell | 21 | 104 | 0.20 | 226 | 46 |
| engager molecule 4 | | | | | |

Legend

| | |
|---|---|
| EpCAM-MSLN T-cell | EpCAM 5-10 × I2Ccc × scFc × H2 × I2Ccc0 |
| engager molecule 1 | |
| EpCAM-MSLN T-cell | EpCAM 5-10 × (EAAAK)₁₀ × I2Ccc × scFc × |
| engager molecule 2 | MSLN H2 × (EAAAK)₁₀ × I2Ccc |
| EpCAM-MSLN T-cell | EpCAM 5-10 × (G₄S)₁₀ × I2Ccc × scFc × MSLN |
| engager molecule 3 | H2 × (G₄S)₁₀ × I2Ccc |
| EpCAM-MSLN T-cell | EpCAM 5-10 × G₄S × I2Ccc × scFc × MSLN |
| engager molecule 4 | H2 × G₄S × I2Ccc |

b.c.t: below calculation threshold

Results: EpCAM-MSLN T-cell engager molecule 1, 2, 3 and 4 showed an increased activity (lower EC50 value) on CHO huEpCAM and huMSLN double positive target cells compared to CHO huEpCAM or CHO huMSLN single positive target cells. EpCAM-MSLN T-cell engager molecule 1, 2 and 3 contain the same target binding and CD3 binding domains in the same orientation [target binding domain×CD3 binding domain×scFc×target binding domain×CD3 binding domain], but they differ in the linker sequences between target binding and CD3 binding domain. Despite these differences in the linker length and sequence, the shown EpCAM-MSLN T-cell engager molecule 1, 2, 3 and 4 show an EC50 selectivity gap between double positive target cells versus single positive target greater than 100-fold.

FIG. 4C: Cytotoxicity curves of CLL1-FLT3 T-cell engager molecules on double positive CHO huCLL1 huFLT3 target cells and single positive CHO huCLL1 or CHO huFLT3 target cells. Effector cells were unstimulated Pan T-cells.

TABLE 15
EC50 values and selectivity gaps of double positive
CHO cells versus single positive CHO cells;

| | CHO | Gap double | CHO | Gap double | CHO |
|---|---|---|---|---|---|
| | huCLL1 | positive | huCLL1 | positive | huFLT3 |
| | EC50 | to single | huFLT3 | to single | EC50 |
| | [pM] | positive | EC50 [pM] | positive | [pM] |
| CLL1-FLT3 T-cell | 130 | 94 | 1.39 | >1000 | b.c.t. |
| engager molecule 1 | | | | | |
| CLL1-FLT3 T-cell | 391 | 98 | 4.00 | >1000 | b.c.t. |
| engager molecule 2 | | | | | |
| CLL1-FLT3 T-cell | 67 | 70 | 0.95 | >1000 | b.c.t. |
| engager molecule 3 | | | | | |
| CLL1-FLT3 T-cell | 177 | 285 | 0.62 | >1000 | b.c.t. |
| engager molecule 4 | | | | | |

Legend

| | |
|---|---|
| CLL1-FLT3 T-cell | CL1 9-G4 CC × I2Ccc × scFc × FL 4-E9 |
| engager molecule 1 | CC × I2Ccc |
| CLL1-FLT3 T-cell | CL1 9-G4 CC × (EAAAK)₁₀ × I2Ccc × G₄ × |
| engager molecule 2 | scFc × G₄ × FL 4-E9 CC × (EAAAK)₁₀ × |
| | I2Ccc |
| CLL1-FLT3 T-cell | CL1 9-G4 CC × G₄S × I2Ccc × G₄ × scFc × |
| engager molecule 3 | G₄ × FL 4-E9 CC × G₄S × I2Ccc |
| CLL1-FLT3 T-cell | CL1 9-G4 CC × (G₄S)₃ × I2Ccc × G₄ × scFc × |
| engager molecule 4 | G₄ × FL 4-E9 CC × (G₄S)₃ × I2Ccc |

b.c.t: below calculation threshold

Results: CLL1-FLT3 T-cell engager molecule 1, 2, 3 and 4 showed an increased activity (lower EC50 value) on CHO huCLL1 and huFLT3 double positive target cells compared to CHO huCLL1 or CHO huFLT3 single positive target cells. CLL1-FLT3 T-cell engager molecule 1, 2, 3 and 4 contain the same target binding and CD3 binding domains in the same orientation [target binding domain×CD3 binding domain×scFc×target binding domain×CD3 binding domain], but differ in the linker sequences between target binding and CD3 binding domain. Despite these differences, the EC50 selectivity gap between double positive target cells versus single positive target cells is comparable for all molecules.

### Example 5: Selectivity Gap of Multitargeting Bispecific T-Cell Engager Polypeptides (MBiTEP) with Different Domains Separating the Two Bispecific Entities

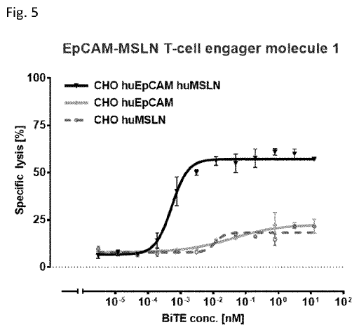

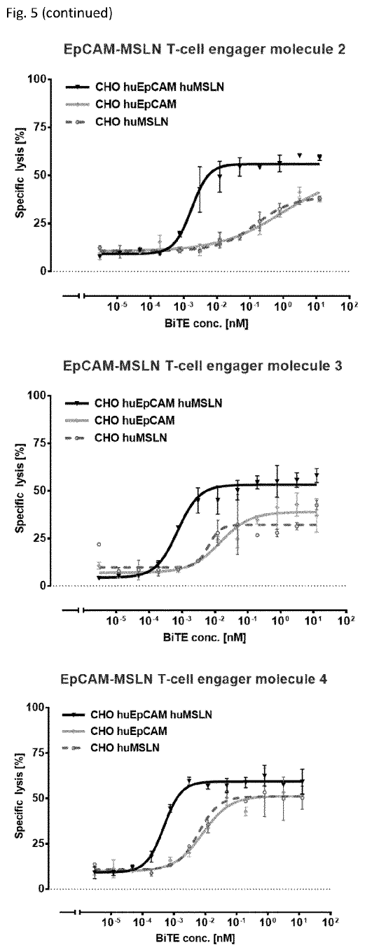

FIG. 5: Cytotoxicity curves of EpCAM MSLN T-cell engager molecules on double positive CHO huEpCAM huMSLN target cells and single positive CHO huEpCAM or CHO huMSLN target cells. Effector cells were unstimulated Pan T-cells.

TABLE 16
Characteristics of structure used between bispecific entities

| | Amino acids | Calculated kDa | Modeled |
|---|---|---|---|
| | between | between | distance between |
| | bispecific | bispecific | bispecific |
| | entities | entities | entities [A] |
| EpCAM-MSLN T-cell | 514 | 54.7 | 130 (rigid) |
| engager molecule 1 | | | |
| EpCAM-MSLN T-cell | 153 | 16.6 | 60-80 (rigid) |
| engager molecule 2 | | | |
| EpCAM-MSLN T-cell | 50 | 3.2 | 30-150 (flexible) |
| engager molecule 3 | | | |
| EpCAM-MSLN T-cell | 5 | 0.3 | 35-40 (flexible) |
| engager molecule 4 | | | |

TABLE 17
EC50 values and selectivity gaps of double positive
CHO cells versus single positive CHO cells;

| | CHO | Gap double | CHO | Gap double | CHO |
|---|---|---|---|---|---|
| | huEpCAM | positive to | huEpCAM | positive to | huMSLN |
| | EC50 [pM] | single | huMSLN | single | EC50 [pM] |
| | | positive | EC50 [pM] | positive | |
| EpCAM-MSLN T-cell | b.c.t. | >1000 | 0.5 | >1000 | b.c.t. |
| engager molecule 1 | | | | | |
| EpCAM-MSLN T-cell | 913 | 155 | 1.7 | 531 | 90 |
| engager molecule 2 | | | | | |
| EpCAM-MSLN T-cell | 14.6 | 7.7 | 0.7 | 20 | 11 |
| engager molecule 3 | | | | | |
| EpCAM-MSLN T-cell | 8.1 | 6.1 | 0.5 | 17 | 13 |
| engager molecule 4 | | | | | |

Legend

| | |
|---|---|
| EpCAM-MSLN T-cell | pCAM 5-10 × I2Ccc × scFc × H2 × I2Ccc0 |
| engager molecule 1 | |
| EpCAM-MSLN T-cell | EpCAM 5-10 × I2Ccc44/100 × G4S × PD1 × G4S × |
| engager molecule 2 | H2 × I2C6cc44/100 |
| EpCAM-MSLN T-cell | EpCAM 5-10 × I2Ccc44/100 × (G4S)10 × H2 × |
| engager molecule 3 | I2C6cc44/100 |
| EpCAM-MSLN T-cell | EpCAM 5-10 × I2Ccc44/100 × H2 × I2C6cc44/100 |
| engager molecule 4 | |

b.c.t: below calculation threshold

Results: The highest selectivity gap between double positive and single positive target cells was achieved by EpCAM-MSLN T-cell engager molecules 1 and 2. In these molecules the bispecific entities were separated by either more than 50 amino acids, OR by an arbitrarily structure with more than 3.2 kDa, OR by an arbitrarily structure that results in a calculated distance/space of at least 40 Å.

### Example 6: Selectivity Gap of Multitargeting Antigen-Binding Molecules of the Invention with Different CD3 Affinities/Activities (Low Vs. High

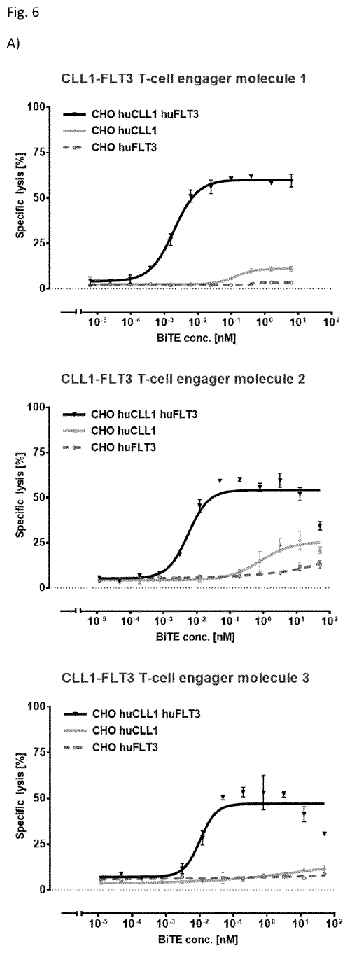

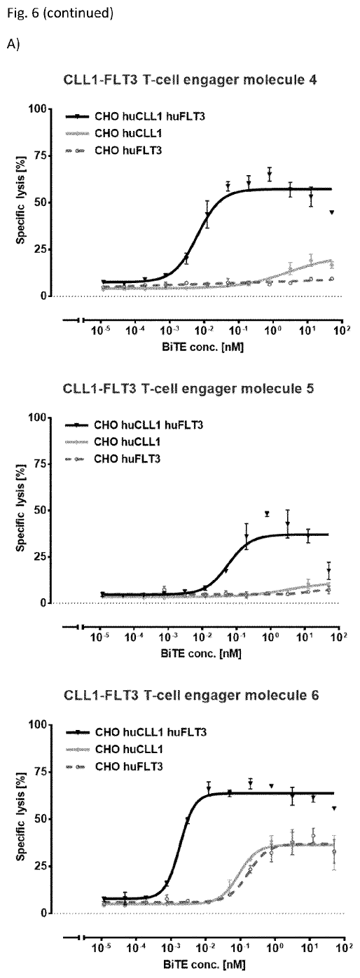

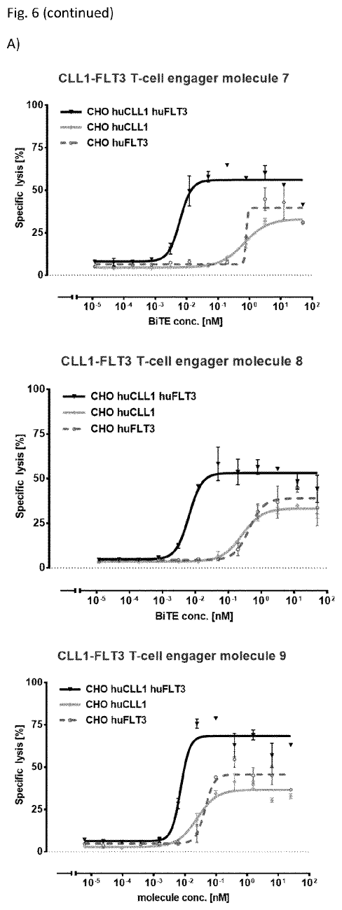

FIG. 6A shows cytotoxicity curves of CLL1-FLT3 T-cell engager molecules on double positive CHO huCLL1 huFLT3 target cells and single positive CHO huCLL1 or CHO huFLT3 target cells. Effector cells were unstimulated Pan T-cells.

TABLE 18
Activity reduction of CD3 binding domains used in CLL1-
FLT3 T-cell engager molecules compared to high affinity CD3
binding domain I2C with K_{D} of 1.2E−08M

| | Activity reduction of |
|---|---|
| | single CD3 binding domain |
| | in molecule compared to |
| | high affinity binding |
| | domain I2C with Kp 1.2E−08M |
| CLL1-FLT3 T-cell engager molecule 1 | Ca 100-fold |
| CLL1-FLT3 T-cell engager molecule 2 | Ca 100-fold |
| CLL1-FLT3 T-cell engager molecule 3 | Ca 92-fold |
| CLL1-FLT3 T-cell engager molecule 4 | Ca 98-fold |
| CLL1-FLT3 T-cell engager molecule 5 | Ca 145-fold |
| CLL1-FLT3 T-cell engager molecule 6 | Ca 6-fold |
| CLL1-FLT3 T-cell engager molecule 7 | Ca 9-fold |
| CLL1-FLT3 T-cell engager molecule 8 | Ca 6-fold |
| CLL1-FLT3 T-cell engager molecule 9 | I2C |

TABLE 19
EC50 values and selectivity gaps of double positive
CHO cells versus single positive CHO cells;

| | CHO | Gap double | CHO | Gap double | CHO |
|---|---|---|---|---|---|
| | huCLL1 | positive | huCLL1 | positive | huFLT3 |
| | EC50 | to single | huFLT3 | to single | EC50 |
| | [pM] | positive | EC50 [pM] | positive | [pM] |
| CLL1-FLT3 T-cell | b.c.t. | >1000 | 1.9 | >1000 | b.c.t. |
| engager molecule 1 | | | | | |
| CLL1-FLT3 T-cell | b.c.t. | >1000 | 5.4 | >1000 | b.c.t. |
| engager molecule 2 | | | | | |
| CLL1-FLT3 T-cell | b.c.t. | >1000 | 10.3 | >1000 | b.c.t. |
| engager molecule 3 | | | | | |
| CLL1-FLT3 T-cell | b.c.t. | >1000 | 6.3 | >1000 | b.c.t. |
| engager molecule 4 | | | | | |
| CLL1-FLT3 T-cell | b.c.t. | >1000 | 56.7 | >1000 | b.c.t. |
| engager molecule 5 | | | | | |
| CLL1-FLT3 T-cell | 89 | 49 | 1.8 | 94 | 170 |
| engager molecule 6 | | | | | |
| CLL1-FLT3 T-cell | 675 | 111 | 6.1 | 129 | 785 |
| engager molecule 7 | | | | | |
| CLL1-FLT3 T-cell | 261 | 41 | 6.4 | 67 | 428 |
| engager molecule 8 | | | | | |
| CLL1-FLT3 T-cell | 24 | 3 | 7.4 | 6 | 43 |
| engager molecule 9 | | | | | |

Legend

| | |
|---|---|
| CLL1-FLT3 T-cell | CL1 9-G4 CC × I2Ccc × scFc × FL 4-E9 |
| engager molecule 1 | CC × I2Ccc |
| CLL1-FLT3 T-cell | CL1 9-G4 CC × I2Ccc × G4 × scFc × |
| engager molecule 2 | G4 × FL 4-E9 CC × I2Ccc |
| CLL1-FLT3 T-cell | CL1 9-G4 CC × 5B1.09 × scFc × FL 4-E9 |
| engager molecule 3 | CC × 5B1.09 |
| CLL1-FLT3 T-cell | CL1 9-G4 CC × 6H10.09 × scFc × FL 4- |
| engager molecule 4 | E9 CC × 6H10.09 |
| CLL1-FLT3 T-cell | CL1 9-G4 CC × 5B1.05 × scFc × FL 4-E9 |
| engager molecule 5 | CC × 5B1.05 |
| CLL1-FLT3 T-cell | CL1 9-G4 CC × 4G10.04 × scFc × FL 4- |
| engager molecule 6 | E9 CC × 4G10.04 |
| CLL1-FLT3 T-cell | CL1 9-G4 CC × 6H10.03 × scFc × FL 4- |
| engager molecule 7 | E9 CC × 6H10.03 |
| CLL1-FLT3 T-cell | CL1 9-G4 CC × 4F10.03mut × scFc × FL |
| engager molecule 8 | 4-E9 CC × 4F10.03mut |
| CLL1-FLT3 T-cell | CL1 9-G4 CC × I2C × (G4S)3 × scFc × |
| engager molecule 9 | (G4S)3 × FL 4-E9 CC × I2C |

b.c.t: below calculation threshold

Results: CLL1-FLT3 T-cell engager molecule 1, 2, 3, 4 and 5 showed the highest selectivity gap between double positive CHO huCLL1 huFLT3 target cells and single positive CHO huCLL1 or huFLT3 target cells, which is over 1000-fold, compared to CLL1-FLT3 T-cell engager molecule 6, 7, 8 and 9. CLL1-FLT3 T-cell engager molecule 1, 2, 3, 4 and 5 contain two CD3-binding domains that are approximately 100-fold less active than the reference CD3 binding domain I2C with an K_{D} of 1.2E-08M. CLL1-FLT3 T-cell engager molecule 6, 7 and 8 contain two CD3-binding domains that are between 6-9-fold less active than I2C. CLL1-FLT3 T-cell engager molecule 9 contains CD3 binding domain I2C.

FIG. 6B shows cytotoxicity curves of EpCAM MSLN T-cell engager molecules on double positive CHO huEpCAM huMSLN target cells and single positive CHO huEpCAM or CHO huMSLN target cells. Effector cells were unstimulated Pan T-cells.

TABLE 20
EC50 values and selectivity gaps of double positive
CHO cells versus single positive CHO cells

| | CHO | Gap double | CHO | Gap double | CHO |
|---|---|---|---|---|---|
| | huEpCAM | positive to | huEpCAM | positive to | huMSLN |
| | EC50 [pM] | single | huMSLN | single | EC50 |
| | | positive | EC50 [pM] | positive | [pM] |
| EpCAM-MSLN T-cell | 151 | 203 | 0.7 | 352 | 263 |
| engager molecule 1 | | | | | |
| EpCAM-MSLN T-cell | 17 | 16 | 1.1 | 10 | 11 |

engager molecule 2

Legend

| | |
|---|---|
| EpCAM-MSLN T-cell | EpCAM 5-10 × I2Ccc × scFc × I2Ccc × H2 |
| engager molecule 1 | |
| EpCAM-MSLN T-cell | EpCAM 5-10 × I2C × scFc × I2C0 × H2 |
| engager molecule 2 | |

Results: EpCAM MSLN T-cell engager molecule 1 showed a higher EC50 selectivity gap between double positive and single positive target cells compared to EpCAM MSLN T-cell engager molecule 2 (203-fold vs 16 fold on CHO huEpCAM compared to double positive cells; and 352-fold vs 10-fold on CHO huMSLN compared to double positive cells). EpCAM-MSLN T-cell engager molecule 2 contains two high affinity CD3-binding domains (I2C, K_{D} of 1.2E-08M), EpCAM-MSLN T-cell engager molecule 1 contains two CD3-binding domains, that are approximately 100-fold less active than I2C.

FIG. 6C shows cytotoxicity curves of CLL1-FLT3 T-cell engager molecules on double positive CHO huCLL1 huFLT3 target cells and single positive CHO huCLL1 or CHO huFLT3 target cells. Effector cells were unstimulated Pan T-cells.

TABLE 21
EC50 values and selectivity gaps of double positive
CHO cells versus single positive CHO cells;

| | | | CHO | | |
|---|---|---|---|---|---|
| | CHO | Gap double | huCLL1 | Gap double | CHO |
| | huCLL1 | positive | huFLT3 | positive to | huFLT3 |
| | EC50 | to single | EC50 | single | EC50 |
| | [pM] | positive | [pM] | positive | [pM] |
| CLL1-FLT3 T-cell | b.c.t. | >1000 | 15.7 | >1000 | b.c.t. |
| engager molecule 1 | | | | | |
| CLL1-FLT3 T-cell | 85 | 28 | 3.1 | 15 | 45 |
| engager molecule 2 | | | | | |

Legend

| | |
|---|---|
| CLL1-FLT3 T-cell | CL1 9-G4 CC × I2Ccc × scFc × I2Ccc × FL |
| engager molecule 1 | 4-E9 CC |
| CLL1-FLT3 T-cell | CL1 9-G4 CC × I2C × scFc × I2C0 × FL |
| engager molecule 2 | 4-E9 CC |

b.c.t: below calculation threshold

Results: CLL1-FLT3 T-cell engager molecule 1 showed a higher selectivity gap between double positive and single positive target cells compared to CLL1-FLT3 T-cell engager molecule 2. CLL1-FLT3 T-cell engager molecule 2 contains two high affinity CD3-binding domains (I2C, K_{D} of 1.2E-08M), CLL1-FLT3 T-cell engager molecule 1 contains two CD3-binding domains, that are approximately 100-fold less active than I2C.

### Example 7 Cytokine Profile of Multitargeting Bispecific T-Cell Engager Polypeptides (MBITEP) with Different CD3 Affinities (Low Vs. High

TABLE 22a
EC50 values are shown of CLL1-FLT3 T-cell engager molecules
on double positive CHO huCLL1 huFLT3 target cells after 48 h.

| | CHO huCLL1 huFLT3 |
|---|---|
| | EC50 [pM] |
| CLL1-FLT3 T-cell engager molecule 1 | 0.4 |
| CLL1-FLT3 T-cell engager molecule 2 | 1.9 |
| CLL1-FLT3 T-cell engager molecule 3 | 0.7 |

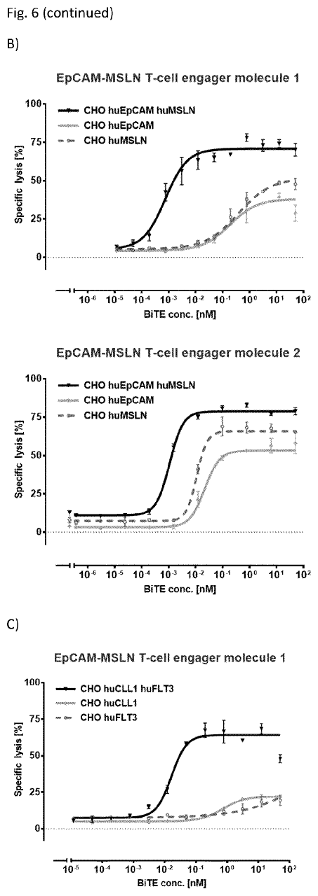

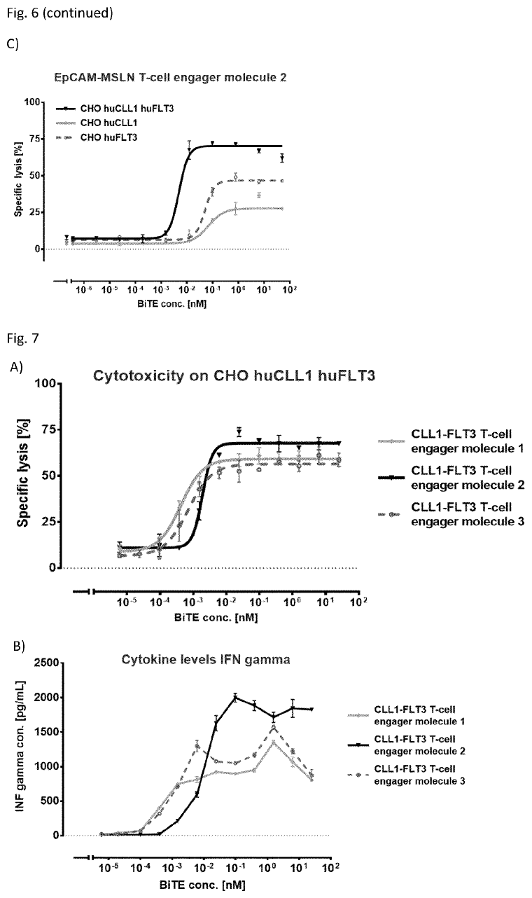

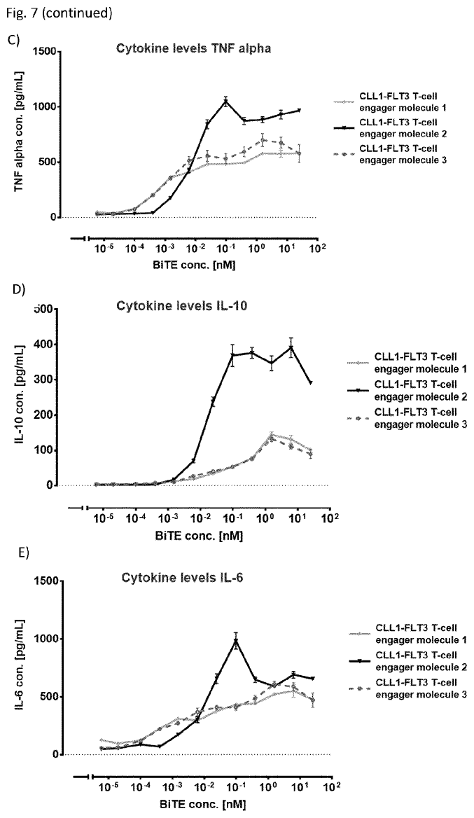

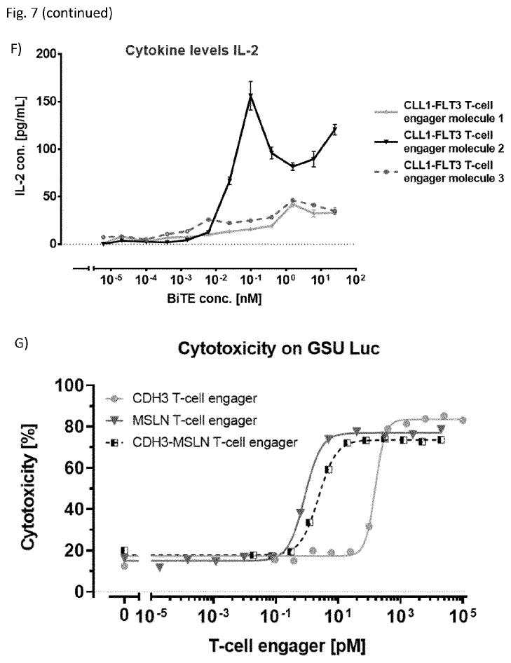

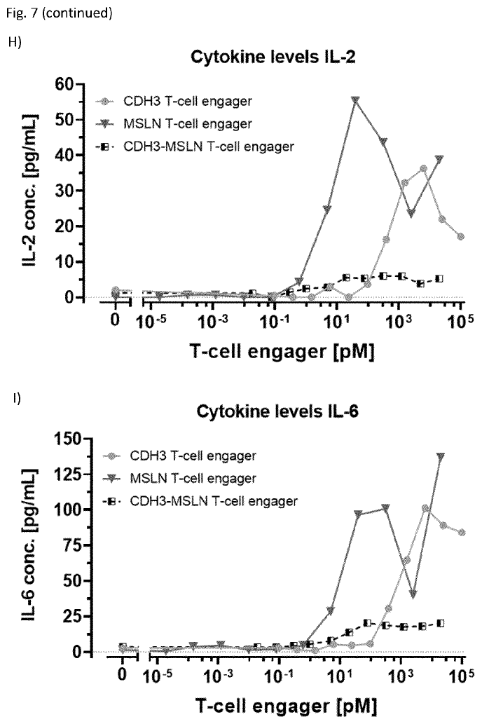

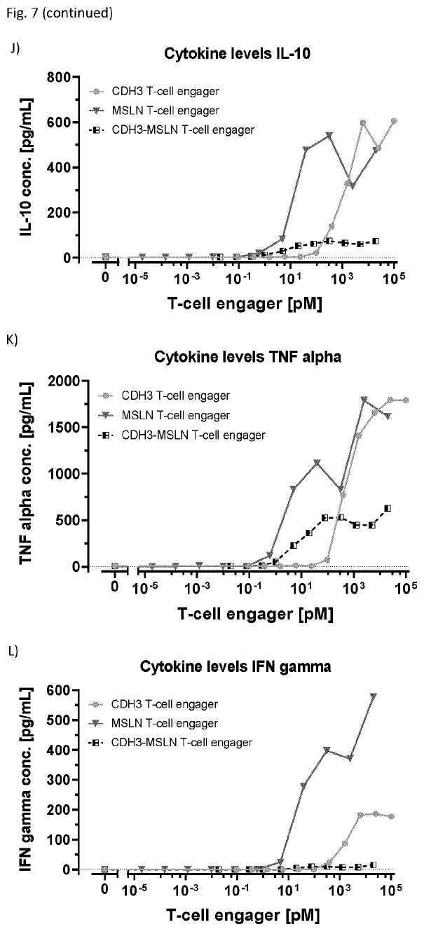

In FIG. 7, cytotoxicity curves are shown of CLL1-FLT3 T-cell engager molecules on double positive CHO huCLL1 huFLT3 target cells after 48 h (FIG. 7A) and released cytokines IL-2, IL-6, IL-10, TNFα und IFNγ after 24 h (FIG. 7B-F). IL-4 was below detection threshold and is therefore not shown. Effector cells were unstimulated PBMC.

TABLE 22b
Activity reduction of anti-CD3 binding domains
used in CLL1-FLT3 T-cell engager molecules compared
to high affinity CD3 binding domain I2C

| | Activity reduction of |
|---|---|
| | single CD3-binding domain |
| | in molecule compared to |
| | high affinity CD3 binding |
| | domain I2C with Kp of 1.2E−08M |
| CLL1-FLT3 T-cell engager | Ca 100-fold |
| molecule 1 | |
| CLL1-FLT3 T-cell engager | I2C |
| molecule 2 | |
| CLL1-FLT3 T-cell engager | Ca 98-fold |
| molecule 3 | |

Legend

| | |
|---|---|
| CLL1-FLT3 T-cell | CL1 9-G4 CC × I2Ccc × scFc × FL 4-E9 |
| engager molecule 1 | CC × I2Ccc |
| CLL1-FLT3 T-cell | CL1 9-G4 CC × I2C × (G4S)3 × scFc × |
| engager molecule 2 | (G4S)3 × FL 4-E9 CC × I2C |
| CLL1-FLT3 T-cell | CL1 9-G4 CC × 6H10.09 × scFc × FL 4-E9 |
| engager molecule 3 | CC × 6H10.09 |

Results: CLL1-FLT3 T-cell engager molecule 1 and 3 showed comparable activity on double positive CHO huCLL1 huFLT3 target cells (0.4 μM and 0.7 μM). CLL1-FLT3 T-cell engager molecule 2 showed a cytotoxic activity of 1.9 μM, which is 4.8-fold and, respectively, 2.7-fold less than molecule 1 and 3. The measured cytokine levels in a cytotoxicity assay with CLL1-FLT3 T-cell engager molecule 2 were higher than CLL1-FLT3 T-cell engager molecules 1 and 3 in all the tested cytokines. CLL1-FLT3 T-cell engager molecule 2 contains two high affinity CD3-binding domains (I2C, K_{D} of 1.2E-08M). CLL1-FLT3 T-cell engager molecule 1 and 3 contain two CD3-binding domains that are approximately 100-fold less active than I2C. Hence, as a general finding, a low affinity CD3 binder can contribute to lower cytokine release.

For corresponding cytotoxicity and cytokine release examination of CDH3-MSLN T-cell engager molecules, GSU Luc Luciferase-transfected cells expressing CDH3 and MSLN were used.

Cytokine release during TDCC in-vitro assay was measured with BD™ Cytometric Bead Array Human Th1/Th2 Cytokine Kit II (BD Biosciences, #551809). Therefore, two cytotoxicity assay sets were set up with full PBMC as effector cells. After 48 h, the supernatant of one assay plate set was removed and analyzed for the levels of human cytokines IL-2, IL-4, IL-6, IL-10, TNFα and IFNγ according to the manufacturer's protocol. After 72 h, the cytotoxic activity of the other assay set was measured.

FIG. 7(G-L): Cytotoxicity curves of CDH3-/MSLN- and CDH3-MSLN T-cell engager molecules on double positive GSU Luc cells after 72 h and released cytokines IL-2, IL-6, IL-10, TNFα und IFNγ after 24 h. Effector cells were unstimulated PBMC.

TABLE 22c
EC50 values are shown of CDH3-/ MSLN- and
CDH3-MSLN T-cell engager molecules on double
positive GSU Luc cells after 72 h
Legend

| | |
|---|---|
| CDH3 T-cell engager | CH3 G8A-BI2 × I2CscFc clone #3 |
| MSLN T-cell engager | MS 5-F11 × I2CO-scFc |
| CDH3-MSLN T-cell engager | CH3 15-E11 CC × I2Lopt × G4 × |
| | scFc × G4 × MS 15-BI2 CC × |
| | I2L_GQ |

| | GSU Luc |
|---|---|
| | EC₅₀ [pM] |
| CDH3 T-cell engager | 155.2 |
| MSLN T-cell engager | 0.84 |
| CDH3-MSLN T-cell engager | 2.33 |

Results: The CDH3-MSLN T-cell engager molecule showed comparable activity on double positive GSU Luc cells as the MSLN T-cell engager (2.33 pM and 0.84 pM). The CDH3 T-cell engager showed a cytotoxic activity of 155.2 pM, which is 67-fold and 185-fold, respectively, less than both other T-cell engagers. The measured cytokine levels in a cytotoxicity assay with the multitargeting CDH3-MSLN T-cell engager molecule were lower than CDH3− or MSLN-monotargeting T-cell engager molecules in all the tested cytokines. Hence, in general, a multitargeting (e.g. CDH3-MSLN) bispecific (T-cell engaging) molecule of the present invention induces less cytokine release than the corresponding mono targeting (e.g. CDH3 and MSLN, respectively) bispecific antigen-binding molecules individually. Therefore, the multitargeting molecule according to the invention is less prone to induce cytokine release-associated side effects which are typically among the most important ones in immunotherapy.

### Example 8: Selectivity Gap of Multitargeting Bispecific T-Cell Engager Molecules (MBiTEM) on Cancer Cell Line

| | | EC50 [pM] |
|---|---|---|
| MSLN-CDH3 T-cell engager molecule 1 on | HCT 116 WT | 0.07 |
| MSLN-CDH3 T-cell engager molecule 1 on | HCT 116 MSLN KO | 15 |
| MSLN-CDH3 T-cell engager molecule 1 on | HCT 116 CDH3 KO | 612 |

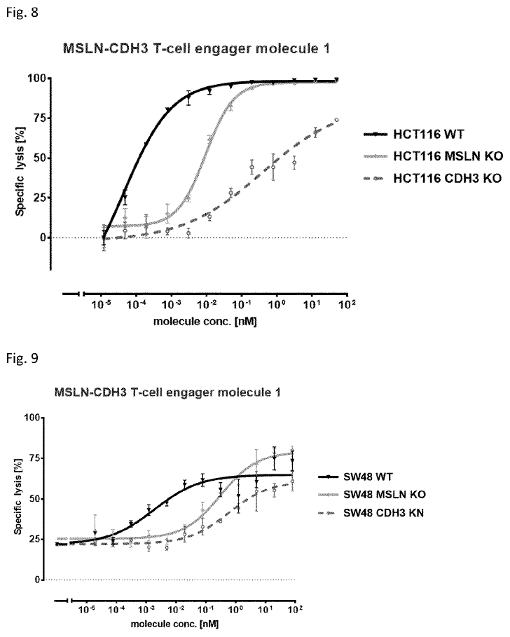

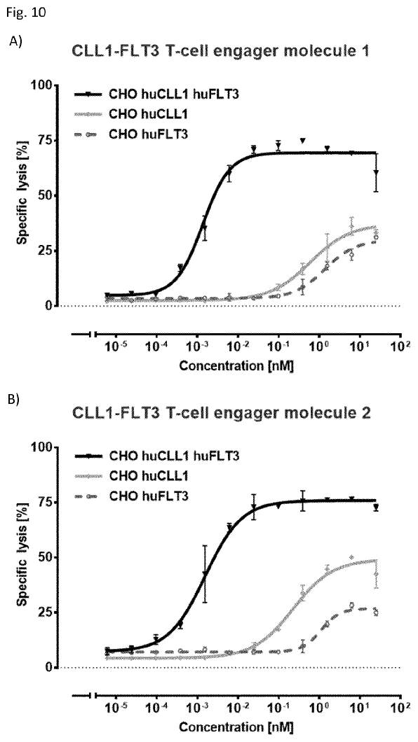

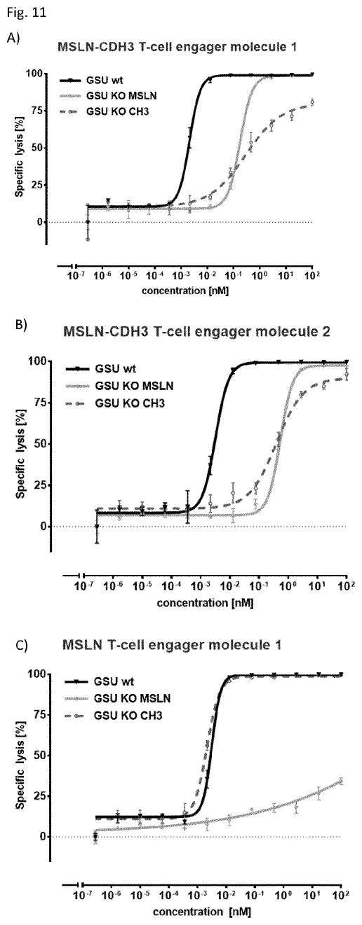

In FIG. 8, cytotoxicity curves and EC50 values are shown of MSLN-CDH3 T-cell engager molecule 1 on double positive cell line HCT 116 (WT) and CDH3 respectively MSLN Knockout (KO) cell lines. Effector cells were unstimulated Pan T-cells.

TABLE 23
EC50 values and selectivity gaps of double positive cell line HCT
116 (WT) and CDH3 respectively MSLN Knockout (KO) cell lines;

| | | Gap double | | Gap double | |
|---|---|---|---|---|---|
| | HCT 116 | positive to | HCT 116 | positive to | HCT 116 |
| | CDH3 KO | single | WT | single | MSLN KO |
| | EC50 [pM] | positive | EC50 [pM] | positive | EC50 [pM] |
| MSLN-CDH3 T-cell | 612 | 8783 | 0.07 | 216 | 15 |
| engager molecule 1 | | | | | |

| | | EC50 [pM] |
|---|---|---|
| MSLN-CDH3 T-cell engager | SW48 WT | 1.8 |
| molecule 1 on | | |
| MSLN-CDH3 T-cell engager | SW48 MSLN KO | 317 |
| molecule 1 on | | |
| MSLN-CDH3 T-cell engager | SW48 CDH3 KO | 598 |
| molecule 1 on | | |

In FIG. 9, cytotoxicity curves and EC50 values are shown of MSLN-CDH3 T-cell engager molecule 1 on double positive cell line SW48 (WT) and CDH3 respectively MSLN Knockout (KO) cell lines. Effector cells were unstimulated Pan T-cells.

TABLE 24
EC50 values and selectivity gaps of double positive cell line
SW48 (WT) and CDH3 respectively MSLN Knockout (KO) cell lines;

| | SW48 | Gap double | | Gap double | SW48 |
|---|---|---|---|---|---|
| | CDH3 KO | positive | SW48 WT | positive | MSLN KO |
| | EC50 | to single | EC50 | to single | EC50 |
| | [pM] | positive | [pM] | positive | [pM] |
| MSLN-CDH3 T-cell | 598 | 326 | 1.8 | 173 | 317 |
| engager molecule 1 | | | | | |

Legend

| | |
|---|---|
| MSLN-CDH3 T-cell | MS 15-B12 × I2C 44/100cc × scFc × |
| engager molecule 1 | CH3 15-E11 × I2C44/100cc0 |

Results: The tested MSLN-CDH3 T-cell engager molecule 1 showed a selectivity gap between double positive target cells and single positive knockout cells over 100-fold on the tested cell lines HCT116 and SW48 and its corresponding knockouts. Cell line HCT116 was measured to have a target antigen copy number level of ca. 2350 Mesothelin Epitopes and ca. 8980 CDH3 Epitopes on each cell's surface. Cell line SW48 has a surface copy number of ca. 4000 Mesothelin Epitopes and 900 CDH3 Epitopes. Independent of the ratios and expression levels of MSLN and CDH3 epitope copy numbers on the target cell surface, the tested MSLN-CDH3 T-cell engager molecule 1 showed a stable selectivity gap >100 on both cell lines.

### Example 9

FACS-Based Cytotoxicity Assay with Unstimulated Human PBMC

### Isolation of Effector Cells

Human peripheral blood mononuclear cells (PBMC) were prepared by Ficoll density gradient centrifugation from enriched lymphocyte preparations (buffy coats), a side product of blood banks collecting blood for transfusions. Buffy coats were supplied by a local blood bank and PBMC were prepared on the day after blood collection. After Ficoll density centrifugation and extensive washes with Dulbecco's PBS (Gibco), remaining erythrocytes were removed from PBMC via incubation with erythrocyte lysis buffer (155 mM NH₄Cl, 10 mM KHCO₃, 100 μM EDTA). Remaining lymphocytes mainly encompass B and T lymphocytes, NK cells and monocytes. PBMC were kept in culture at 37° C./5% CO₂ in RPMI medium (Gibco) with 10% FBS (Bio West, #S1810).

### Isolation of Human T-Cells

For isolation of human T-cells, Pan T Cell Isolation Kit, human (Miltenyi Biotec, MACS, #130-096-535) was used to deplete non-target cells, i.e., monocytes, neutrophils, eosinophils, B cells, stem cells, dendritic cells, NK cells, granulocytes, or erythroid cells from the PBMC cell solution. Therefore, respective number of PBMC was centrifuged for 10 min at room temperature at 300×g. Supernatant was discarded, and the cell pellet was resuspended in MACS isolation buffer (Dulbecco's PBS (Gibco), 100 μM EDTA, 0.5% FBS (Bio West, #S1810)) [40 μl buffer/1×10⁷ cells]. Pan T Cell Biotin-Antibody cocktail [10 μL/1×10⁷ cells] was added and suspension was incubated for 5 min at 4° C. Afterwards, MACS isolation buffer was added [30 μl buffer/1×10⁷ cells] together with Anti-Biotin MicroBeads [20 μl/1×10⁷ cells)] and cell suspension was left at 4° C. for 10 min. The cell solution was then applied to LS Columns (Miltenyi Biotec, #130-042-401) in the magnetic field of a suitable Miltenyi Separator to isolate untouched T cells while magnetically labelled non-T-cells remain on the column. Columns were washed 3 times with MACS isolation buffer. Column flowthrough was centrifued (see above), supernatant was discarded and cells were resuspended in RPMI complete medium i.e. RPMI1640 (Biochrom AG, #FG1215) supplemented with 10% FBS (Bio West, #S1810), 1× non-essential amino acids (Biochrom AG, #K0293), 1 mM sodium pyruvate (Biochrom AG, #L0473) and 100 U/mL penicillin/streptomycin (Biochrom AG, #A2213) and incubated at 37° C. until needed.

### Target Cell Labeling for Flow-Cytometry Based T-Cell-Dependent Cellular Cytotoxicity (TDCC) Assay

For the analysis of cell lysis in flow cytometry assays, the fluorescent membrane dye DiOC₁₈ (DiO) (Thermo Fisher, #V22886) was used to label human-target transfected CHO cells or cancer cell lines as target cells and distinguish them from effector cells. Briefly, cells were harvested, washed once with PBS and adjusted to 10⁶ cell/mL in PBS containing the membrane dye DiO (5 μL/10⁶ cells). After incubation for 3 min at 37° C., cells were washed twice in complete RPMI medium and directly used in assay.

### Setup of Flow Cytometry-Based T-Cell-Dependent Cellular Cytotoxicity (TDCC) Assay and Analysis

Cytotoxic activity of T-cell engager molecules of the invention was determined through the capability of inducing T-cell mediated target cell lysis. Therefore, the lysis of human target cells in the presence of serial dilutions of bispecific T-cell engager molecules and effector cells was analyzed.

DiO-labeled target-cells and effector cells (i.e., Pan T-cells) were mixed at an effector to target-cell (E:T) ratio of 10:1 and incubated with serial dilutions of the corresponding bispecific T-cell engager molecule in 96-well plates. Plates were incubated at 37° C., 5% CO2 and 95% relative humidity for 48 h. On day of assay analysis, cells were transferred to a new 96-well plate and loss of target cell membrane integrity was monitored by adding propidium iodide (PI) at a final concentration of 1 μg/mL. PI is a membrane impermeable dye that normally is excluded from viable cells, whereas dead cells take it up and become identifiable by fluorescent emission.

Samples were measured by flow cytometry on an iQue Plus (Intellicyt, now Sartorius) instrument and analyzed by Forecyt software (Intellicyt). Target cells were identified as DiO-positive cells. PI-negative target cells were classified as living target cells. Percentage of specific cell lysis respective cytotoxicity was calculated according to the following formula:

$$Cytoxicity [ \% ] = ( \frac{{n}_{dead target cells}}{{n}_{target cells}} ) \times 100$$

$$n = number of events per well$$

In some experiments, the cytotoxicity was calculated according to this formula:

$$Cytoxicity [ \% ] = ( 1 - \frac{{\%}_{viable target cells BiTE treated}}{{\%}_{viable target cells untreated}} ) \times 100$$

$${\%}_{viable target cells} = ( 1 - \frac{{n}_{dead target cells}}{{n}_{target cells}} ) \times 100$$

$$n = number of events per well$$

Using GraphPad Prism 7.04 software (Graph Pad Software, San Diego), the percentage of cytotoxicity was plotted against the corresponding bispecific T-cell engager molecule concentrations. Sigmoidal dose response curves were analyzed with the four parametric logistic regression models with variable slope and EC50 values were calculated.

The following target cell lines were used for the FACS-based cytotoxicity assay:
CHO huMSLN:
   Parental CHO (DHFR) cells transfected with human MSLN on pEFDHFR-MTX1 for expression of human MSLN and dummy sequence on pEFDHFR-MTX2
CHO huEpCAM
   Parental CHO (DHFR) cells transfected with human EpCAM on pEFDHFR-MTX2 for expression of human EpCAM and dummy sequence on pEFDHFR-MTX1
CHO huMSLN huEpCAM
   Parental CHO (DHFR) cells transfected with human MSLN on pEFDHFR-MTX1 and human EpCAM on pEFDHFR-MTX2 for simultaneous expression of human MSLN and human EpCAM
CHO huCLL1
   Parental CHO (DHFR) cells transfected with human CLL1 on pEFDHFR for expression of human CLL1
CHO huFLT3
   Parental CHO (DHFR) cells transfected with human FLT3 on pEFDHFR for expression of human FLT3
CHO huCLL1 huFLT3
   Parental CHO (DHFR) cells transfected with human CLL1 on pEFDHFR-MTX1 and human FLT3 on pEFDHFR-MTX2 for simultaneous expression of human CLL1 and human FLT3
SW48 WT
   Parental cell line, wildtype (WT)
SW48 MSLN KO
   Parental cell line SW48, in which MSLN gene was knocked out (KO)
SW48 CDH3 KO
   Parental cell line SW48, in which CDH3 gene was knocked out (KO)

### Cytokine Measurement of In Vitro TDCC Assay

Cytokine release during TDCC in-vitro assay was measured with BD™ Cytometric Bead Array Human Th1/Th2 Cytokine Kit II (BD Biosciences, #551809). Therefore, two cytotoxicity assay sets were set up with full PBMC as effector cells. After 24 h, the supernatant of one assay plate set was removed and analyzed for the levels of human cytokines IL-2, IL-4, IL-6, IL-10, TNFα und IFNγ according to the manufacturer's protocol. After 48 h, the cytotoxic activity of the other assay set was measured.

### Setup of Luciferase-Based T-Cell-Dependent Cellular Cytotoxicity (TDCC) Assay and Analysis

Luc-positive target-cells and effector cells (i.e., Pan T-cells) were mixed at an effector to target-cell (E:T) ratio of 10:1 and incubated with serial dilutions of the corresponding bispecific T-cell engager molecule in 384-well plates. The multitargeting bispecific antigen-binding molecule-mediated cytotoxic reaction proceeded for 48 hours in a 5% CO₂ humidified incubator. Then 25 μL substrate (Steady-Glo® Reagent, Promega) were transferred to the 384-well plate. Only living, luciferase-positive cells react to the substrate and thus create a luminescence signal. Samples were measured with a SPARK microplate reader (TECAN) and analyzed by Spark Control Magellan software (TECAN).

Percentage of cytotoxicity was calculated as follows:

$$Cytoxicity [ \% ] = ( 1 - \frac{R L {U}_{Sample}}{R L {U}_{N egative Control}} ) \times 100$$

RLU=relative light units

Negative-Control=cells without multi-specific antigen-binding molecule

Using GraphPad Prism 7.04 software (Graph Pad Software, San Diego), the percentage of cytotoxicity was plotted against the corresponding bispecific T-cell engager molecule concentrations. Sigmoidal dose response curves were analyzed with the four parametric logistic regression models with variable slope and EC50 values were calculated.

Following target cell lines were used for the Luciferase-based cytotoxicity assay:
HCT 116 LUC WT
   Parental cell line, wildtype (WT), transfected with luciferase
HCT 116 LUC MSLN KO
   Parental cell line HCT 116 LUC, in which MSLN gene was knocked out (KO)
HCT 116 LUC CDH3 KO
   Parental cell line HCT 116 LUC, in which CDH3 gene was knocked out (KO)
GSU LUC WT
   Parental cell line, wildtype (wt), transfected with luciferase
GSU LUC MSLN KO
   Parental cell line GSU LUC wt, in which MSLN gene was knocked out (KO)
GSU LUC CDH3 KO
   Parental cell line GSU LUC wt, in which CDH3 gene was knocked out (KO)

### Selectivity Gap of Different Multitargeting Antigen-Binding Molecules

TABLE 25
EC50 values and selectivity gaps of double positive
CHO cells versus single positive CHO cells;

| | | | CHO | | |
|---|---|---|---|---|---|
| | CHO | Gap double | huCLL1 | Gap double | CHO |
| | huCLL1 | positive | huFLT3 | positive | huFLT3 |
| | EC50 | to single | EC50 | to single | EC50 |
| | [pM] | positive | [pM] | positive | [pM] |
| CLL1-FLT3 T-cell | 1198 | 437 | 2.7 | >437 | b.c.t. |
| engager molecule 1 | | | | | |
| CLL1-FLT3 T-cell | 454 | 146 | 3.1 | 706 | 2190 |
| engager molecule 2 | | | | | |

Legend

| | |
|---|---|
| CLL1-FLT3 T-cell | CL1 9-G4 CC × I2C cc × scFc × |
| engager molecule 1 | FL 4-E9 CC × I2C cc |
| CLL1-FLT3 T-cell | I2Ccc × CL1 9-G4 CC × scFc × |
| engager molecule 2 | FL 4-E9 CC × I2Ccc |

b.c.t: below calculation threshold

Results: CLL1-FLT3 T-cell engager molecule 1 and 2 showed an increased activity (lower EC50 value) on huCLL1 and huFLT3 double positive target cells compared to huCLL1 or huFLT3 single positive target cells. Those molecules showed EC50 selectivity gaps greater 100-fold on double positive target cells versus single positive target cells. CLL1-FLT3 T-cell engager molecule 1 and 2 both have one bispecific entity (one target binding domain and one CD3 binding domain) at the N-terminus and one bispecific entity at the C-terminus, separated by a scFc domain. They differ in the domain arrangement, CLL1-FLT3 T-cell engager molecule 1 has the following arrangement: [target binding domain×CD3 binding domain×scFc×target binding domain×CD3 binding domain], CLL1-FLT3 T-cell engager molecule 2 comprises [CD3 binding domain×target binding domain×scFc×target binding domain×CD3 binding domain].

### Example 10: Selectivity Gap of Single-Chain Multitargeting Bispecific T-Cell Engager Polypeptides Vs. Dual-Chain Multitargeting Bispecific T-Cell Engager Polypeptides

TABLE 26
EC50 values in pM and gaps of naïve
GSU cells versus target knockout GSU cells.

| | EC50 | | EC50 | | EC50 |
|---|---|---|---|---|---|
| | GSU KO | | GSU | | GSU KO |
| | CDH3 | fold | wt | fold | MSLN |
| | [pM] | gap | [pM] | gap | [pM] |
| MSLN-CDH3 T-cell | 289 | 138 | 2.1 | 84 | 177 |
| engager molecule 1 | | | | | |
| MSLN-CDH3 T-cell | 391 | 121 | 3.2 | 165 | 533 |
| engager molecule 2 | | | | | |
| MSLN T-cell | 2.1 | 1 | 3.1 | — | b.c.t. |
| engager molecule 1 | | | | | |
| CDH3 T-cell | b.c.t. | — | 201 | 1 | 180 |
| engager molecule 1 | | | | | |

b.c.t: below calculation threshold

The tested MSLN-CDH3 T-cell engager molecules 1 and 2 showed increased activity (lower EC50 values) on MSLN and CDH3 double positive GSU wt cells compared to respective GSU KO cells (GSU KO CDH3 and GSU KO MSLN). These molecules showed EC50 selectivity gaps of at least 80-fold on double positive target cells versus single positive target cells. MSLN-CDH3 T-cell engager molecules 1 comprises one multitargeting bispecific T-cell engager polypeptide, whereas MSLN-CDH3 T-cell engager molecule 2 comprises a heterodimer of two different (in combination) multitargeting bispecific T-cell engager polypeptides. Both have the domain arrangement of [target binding domain×CD3 binding domain×spacer×target binding domain×CD3 binding domain]. Mono targeting control T-cell engager molecules had comparable activity on single positive vs. double positive cells (selectivity gap of ˜1).

Legend

| | |
|---|---|
| MSLN-CDH3 T-cell | MS 15-B12 CC × I2L × G4 × scFc × |
| engager molecule 1 | G4 × CH3 15-E11 CC × I2L |
| MSLN-CDH3 T-cell | MS 15-B12 CC × 6H10.09 × (G4) × heFc (A) * |
| engager molecule 2 | heFc (B) × (G4) × CH3 15-E11 CC × 6H10.09 |
| | (Seq ID 311 + 312) |
| MSLN T-cell | MSLN 5F11 × I2C -scFc |
| engager molecule 1 | |
| CDH3 T-cell | CH3 G8A 6-B12 × I2C0-scFc |
| engager molecule 1 | |

Example 11 Selectivity gap of multitargeting bispecific T-cell engager polypeptides (MBiTEP) with different spacers separating the two bispecific entities

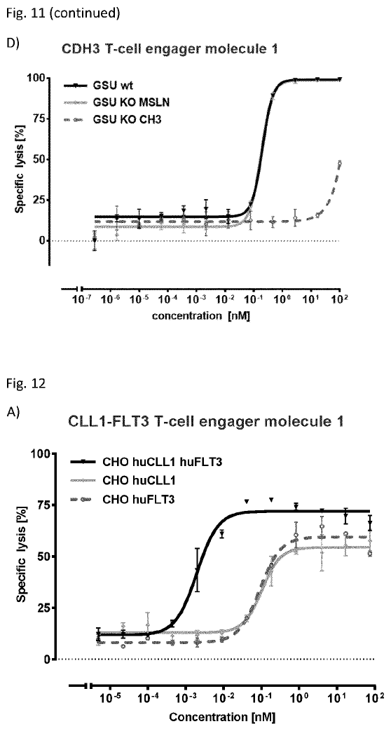

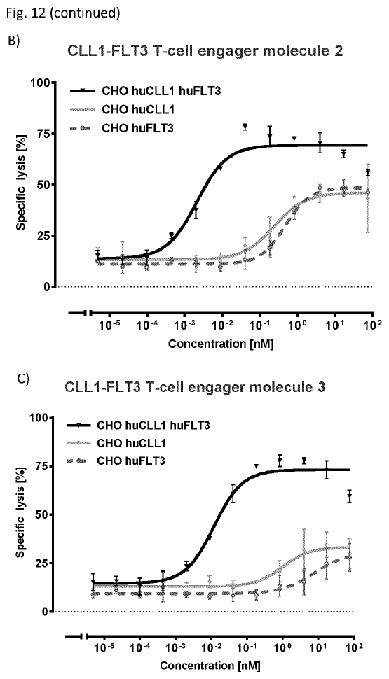

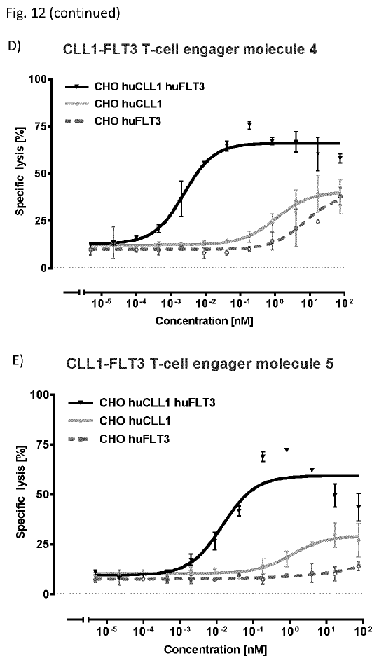

FIG. 12(A-E) shows cytotoxicity curves and EC50 values of CLL1-FLT3 T-cell engager molecules on double positive CHO huCLL1 huFLT3 target cells and single positive CHO huCLL1 or CHO huFLT3 target cells. Effector cells were unstimulated Pan T-cells.

TABLE 27
EC50 values and selectivity gaps of double positive
CHO cells versus single positive CHO cells;

| | | | CHO | | |
|---|---|---|---|---|---|
| | CHO | Gap double | huCLL1 | Gap double | CHO |
| | huCLL1 | positive | huFLT3 | positive | huFLT3 |
| | EC50 | to single | EC50 | to single | EC50 |
| | [pM] | positive | [pM] | positive | [pM] |
| CLL1-FLT3 T-cell | 135.3 | 50 | 2.7 | 36 | 97.7 |
| engager molecule 1 | | | | | |
| CLL1-FLT3 T-cell | 180 | 74 | 2.4 | 189 | 462 |
| engager molecule 2 | | | | | |
| CLL1-FLT3 T-cell | b.c.t. | >100 | 14.5 | >100 | b.c.t. |
| engager molecule 3 | | | | | |
| CLL1-FLT3 T-cell | 1919 | 834 | 2.3 | >100 | b.c.t. |
| engager molecule 4 | | | | | |
| CLL1-FLT3 T-cell | b.c.t. | >100 | 18.7 | >100 | b.c.t. |
| engager molecule 5 | | | | | |

Legend

| | |
|---|---|
| CLL1-FLT3 T-cell | CL1 9-G4CC × I2C CC × |
| engager molecule 1 | FL 4-E9CC × I2C |
| CLL1-FLT3 T-cell | CL1 9-G4CC × I2C CC × (EAAAK)10 × |
| engager molecule 2 | FL 4-E9CC × I2C |
| CLL1-FLT3 T-cell | CL1 9-G4CC × I2C CC × HSA × |
| engager molecule 3 | FL 4-E9CC × I2C |
| CLL1-FLT3 T-cell | CL1 9-G4CC × I2C CC × scFc × |
| engager molecule 4 | FL 4-E9CC × I2C |
| CLL1-FLT3 T-cell | CL1 9-G4CC × I2C CC × scFc × |
| engager molecule 5 | scFc2 × FL 4-E9CC × I2C |

b.c.t: below calculation threshold

Results: CLL1-FLT3 T-cell engager molecule 1, 2, 3, 4 and 5 contain the same target binding and CD3 binding domains in the same arrangement [target binding domain×CD3 binding domain×Spacer×target binding domain×CD3 binding domain], but differ in the spacer domain between the bispecific entities. EC50 selectivity gaps between double positive target cells versus single positive target cells greater than 100-fold were seen with CLL1-FLT3 T-cell engager molecule 3, 4 and 5, in which the bispecific entities were separated by spacers of more than 50 amino acids and 5 kDa to provide a center of mass distance of at least about 50 Å. The best combination of selectivity gaps and overall activity on double positive target cells is seen with CLL1-FLT3 T-cell engager molecule 4, where the bispecific entities are separated by 514 amino acids or 54.7 kDa or 101 Å; CLL1-FLT3 T-cell engager molecule 3 and 5 with spacer of 615 amino acids/68.3 kDA/114 Å respectively 998 amino acids/107.5 kDA/153 Å show a slight reduction in overall activity, but still maintain a selectivity gap greater than 100-fold.

TABLE 28
Characteristics of structures used between bispecifc entities

| | | Amino acids | Calculated | Calculated |
|---|---|---|---|---|
| | Spacer | between | kDa between | Center-of- |
| | between | bispecific | bispecific | mass median |
| | entities | entities | entities | distance [Å] |
| CLL1-FLT3 T-cell | G4S | 5 | 0.3 | 47 |
| engager molecule 1 | | | | |
| CLL1-FLT3 T-cell | (EAAAK)₁₀ | 50 | 4.7 | 47 |
| engager molecule 2 | | | | |
| CLL1-FLT3 T-cell | (G₄S)₃ HSA | 615 | 68.3 | 114 |
| engager molecule 3 | (G₄S)₃ | | | |
| CLL1-FLT3 T-cell | (G₄S)₃ scFc | 514 | 54.7 | 101 |
| engager molecule 4 | (G₄S)₃ | | | |
| CLL1-FLT3 T-cell | (G₄S)₃ | 998 | 107.5 | 153 |
| engager molecule 5 | scFc-scFc | | | |
| | (G₄S)₃ | | | |

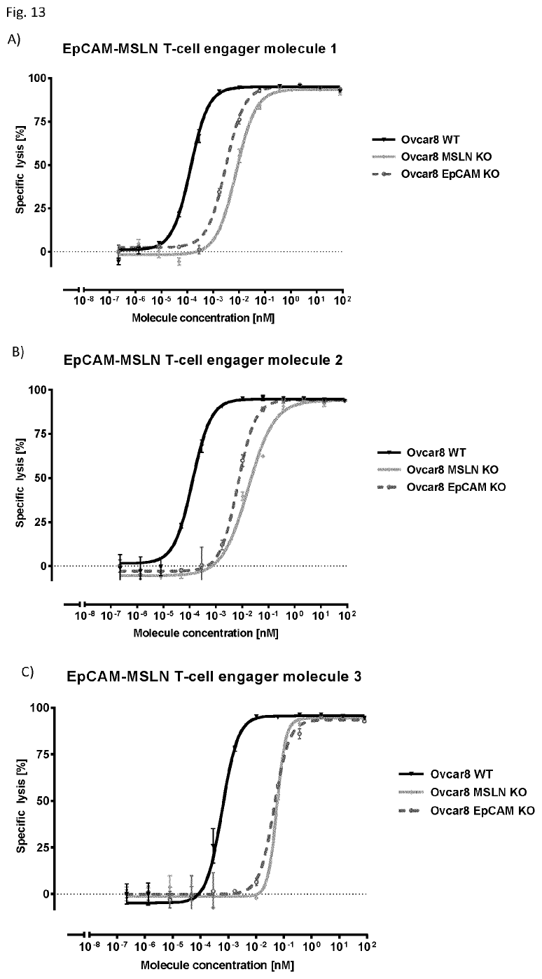

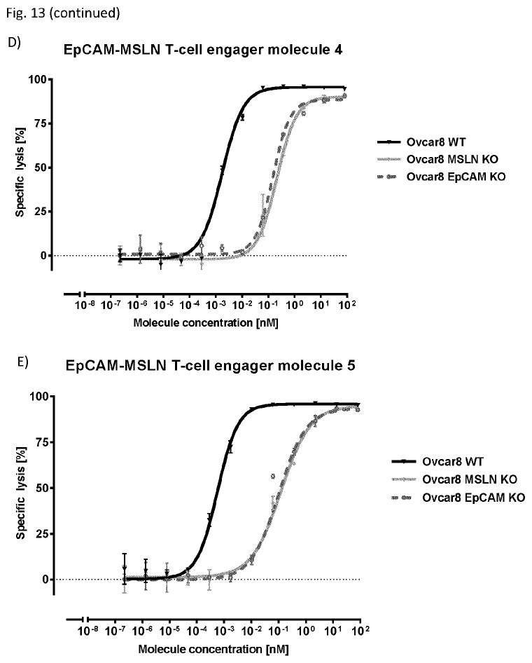

FIG. 13 (A-E) shows cytotoxicity curves of EpCAM-MSLN T-cell engager molecules on double positive Ovcar8 Wildtype cells and single positive Ovcar8 MSLN KO or Ovcar8 EpCAM KO target cells. Effector cells were unstimulated Pan T-cells.

TABLE 29
EC50 values and selectivity gaps of double positive Ovcar8
WT cells versus single positive Ovcar8 KO cells.

| | Ovcar8 | Gap double | Ovcar8 | Gap double | Ovcar8 |
|---|---|---|---|---|---|
| | EpCAM KO | positive to | WT | positive to | MSLN KO |
| | EC50 | single | EC50 | single | EC50 |
| | [pM] | positive | [pM] | positive | [pM] |
| EpCAM-MSLN T-cell | 3.1 | 23 | 0.13 | 59 | 7.7 |
| engager molecule 1 | | | | | |
| EpCAM-MSLN T-cell | 6.7 | 53 | 0.13 | 133 | 17 |
| engager molecule 2 | | | | | |
| EpCAM-MSLN T-cell | 46 | 75 | 0.6 | 93 | 57 |
| engager molecule 3 | | | | | |
| EpCAM-MSLN T-cell | 149 | 87 | 1.7 | 124 | 212 |
| engager molecule 4 | | | | | |
| EpCAM-MSLN T-cell | 101 | 177 | 0.6 | 213 | 121 |
| engager molecule 5 | | | | | |

| | | Delete | |
|---|---|---|---|
| Legend | | column | |
| EpCAM-MSLN | T-cell | F8C | EpCAM 5-10 × I2Ccc × H2 × |
| engager molecule 1 | | | I2Ccc |
| EpCAM-MSLN | T-cell | W4F | EpCAM 5-10 × I2Ccc × (G4S)10 × |
| engager molecule 2 | | | H2 × I2C6cc44/100 |
| EpCAM-MSLN | T-cell | S2F | EpCAM 5-10 × I2Ccc × PD1 × |
| engager molecule 3 | | | H2 × I2Ccc |
| EpCAM-MSLN | T-cell | J9S | EpCAM 5-10 × I2Ccc × HSA × |
| engager molecule 4 | | | H2 × I2Ccc |
| EpCAM-MSLN | T-cell | F7W | EpCAM 5-10 × I2Ccc × scFc × |
| engager molecule 5 | | | H2 × I2Ccc |

Results: EpCAM-MSLN T-cell engager molecule 1, 2, 3, 4 and 5 contain the same target binding and CD3 binding domains in the same arrangement [target binding domain×CD3 binding domain×Spacer×target binding domain×CD3 binding domain], but differ in the spacer domains between the bispecific entities. When comparing EpCAM-MSLN T-cell engager molecule 1, 2, 3, 4 and 5, the selectivity gap between double positive target cells versus single positive target cells gets better with increasing spacer separating the bispecific entities, with the best result of >100-fold for EpCAM MSLN T-cell engager molecule 5, where the bispecific entities are separated by 514 amino acids/54.7 kDa/101 Å.

TABLE 30
Characteristics of structure used between bispecific entities

| | | Amino acids | Calculated | Calculated |
|---|---|---|---|---|
| | Spacer | between | kDa between | Center-of- |
| | between | bispecific | bispecific | mass median |
| | entities | entities | entities | distance [Å] |
| EpCAM-MSLN T-cell | G₄S | 5 | 0.3 | 47 |
| engager molecule 1 | | | | |
| EpCAM-MSLN T-cell | (G₄S)₁₀ | 50 | 3.2 | 48 |
| engager molecule 2 | | | | |
| EpCAM-MSLN T-cell | G₄S PD1 G₄S | 153 | 16.6 | 86 |
| engager molecule 3 | | | | |
| EpCAM-MSLN T-cell | (G₄S)₃ HSA (G₄S)₃ | 615 | 68.3 | 114 |
| engager molecule 4 | | | | |
| EpCAM-MSLN T-cell | (G₄S)₃ scFc (G₄S)₃ | 514 | 54.7 | 101 |
| engager molecule 5 | | | | |

### Example 12 Luciferase-Based Cytotoxicity Assay with Unstimulated Human PBMC

### Isolation of Effector Cells

Human peripheral blood mononuclear cells (PBMC) were prepared by Ficoll density gradient centrifugation from enriched lymphocyte preparations (buffy coats), a side product of blood banks collecting blood for transfusions. Buffy coats were supplied by a local blood bank and PBMC were prepared on the day after blood collection. After Ficoll density centrifugation and extensive washes with Dulbecco's PBS (Gibco), remaining erythrocytes were removed from PBMC via incubation with erythrocyte lysis buffer (155 mM NH₄Cl, 10 mM KHCO₃, 100 μM EDTA). Remaining lymphocytes mainly encompass B and T lymphocytes, NK cells and monocytes. PBMC were kept in culture at 37° C./5% CO₂ in RPMI medium (Gibco) with 10% FCS (Gibco).

Depletion of CD14⁺ and CD56⁺ Cells

For depletion of CD14+ cells, human CD14 MicroBeads (Milteny Biotec, MACS, #130-050-201) were used, for depletion of NK cells human CD56 MicroBeads (MACS, #130-050-401). PBMC were counted and centrifuged for 10 min at room temperature with 300×g. The supernatant was discarded and the cell pellet resuspended in MACS isolation buffer (60 μL/10⁷ cells). CD14 MicroBeads and CD56 MicroBeads (20 μL/10⁷ cells) were added and incubated for 15 min at 4-8° C. The cells were washed with AutoMACS rinsing buffer (Milteny #130-091-222) (1-2 mL/10⁷ cells). After centrifugation (see above), supernatant was discarded and cells resuspended in MACS isolation buffer (500 μL/10⁸ cells). CD14/CD56 negative cells were then isolated using LS Columns (Milteny Biotec, #130-042-401). PBMC w/o CD14+/CD56+ cells were adjusted to 1.2×10⁶ cells/mL and cultured in RPMI complete medium i.e. RPMI1640 (Biochrom AG, #FG1215) supplemented with 10% FBS (Bio West, #S1810), 1× non-essential amino acids (Biochrom AG, #K0293), 10 mM Hepes buffer (Biochrom AG, #L1613), 1 mM sodium pyruvate (Biochrom AG, #L0473) and 100 U/mL penicillin/streptomycin (Biochrom AG, #A2213) at 37° ° C. in an incubator until needed.

### Target Cell Preparation

Cells were harvested, spinned down and adjusted to 1.2×10⁵ cells/mL in complete RPMI medium. The vitality of cells was determined using Nucleocounter NC-250 (Chemometec) and Solution18 Dye containing Acridine Orange and DAPI (Chemometec).

### Luciferase Based Analysis

This assay was designed to quantify the lysis of target cells in the presence of serial dilutions of multi-specific antibody constructs. Equal volumes of Luciferase-positive target cells and effector cells (i.e., PBMC w/o CD14+; CD56+ cells) were mixed, resulting in an E:T cell ratio of 10:1. 42 μL of this suspension were transferred to each well of a 384-well plate. 8 μL of serial dilutions of the corresponding multi-specific antibody constructs and a negative control antibody constructs (a CD3-based antibody construct recognizing an irrelevant target antigen) or RPMI complete medium as an additional negative control were added. The multi-specific antibody-mediated cytotoxic reaction proceeded for 48 hours in a 5% CO₂ humidified incubator. Then 25 μL substrate (Steady-Glo® Reagent, Promega) were transferred to the 384-well plate. Only living, Luciferase-positive cells react to the substrate and thus create a luminescence signal. Samples were measured with a SPARK microplate reader (TECAN) and analyzed by Spark Control Magellan software (TECAN).

Percentage of cytotoxicity was calculated as follows:

$$Cytoxicity [ \% ] = ( 1 - \frac{R L {U}_{Sample}}{R L {U}_{Negative ‐ Control}} ) \times 100$$

RLU=relative light units

Negative-Control=cells without multi-specific antibody construct

Using GraphPad Prism 7.04 software (Graph Pad Software, San Diego), the percentage of cytotoxicity was plotted against the corresponding multi-specific antibody construct concentrations. Dose response curves were analyzed with the four parametric logistic regression models for evaluation of sigmoid dose response curves with fixed hill slope and EC50 values were calculated.

Following target cell lines were used for the Luciferase-based cytotoxicity assay:
GSU-LUC wt (CDH3+ and MSLN+)
GSU-LUC KO CDH3 (CDH3− and MSLN+)
GSU-LUC KO MSLN (CDH3+ and MSLN−)
HCT 116-LUC wt (CDH3+ and MSLN+)
HCT 116-LUC KO CDH3 (CDH3− and MSLN+)
HCT 116-LUC KO MSLN (CDH3+ and MSLN−)

| | EC50 |
|---|---|
| | [pM] |
| MSLN-CDH3 T-cell engager molecule 1 on GSU wt | 1.34 |
| MSLN-CDH3 T-cell engager molecule 1 on GSU KO CDH3 | 511.8 |
| MSLN-CDH3 T-cell engager molecule 1 on GSU KO MSLN | 3243.2 |

| | EC50 |
|---|---|
| | [pM] |
| MSLN-CDH3 T-cell engager molecule 2 on GSU wt | 0.35 |
| MSLN-CDH3 T-cell engager molecule 2 on GSU KO CDH3 | 75.2 |
| MSLN-CDH3 T-cell engager molecule 2 on GSU KO MSLN | 244.7 |

### Legend:

MSLN-CDH3 T-cell engager molecule 1: CH3 15-E11 CC×I2L×G4×scFc xG4×MS 15-B12 CC×I2L_(SEQ ID NO 251)

MSLN-CDH3 T-cell engager molecule 2: CH3 15-E11 VAG CC×I2L×G4×scFc×MS 15-B12 CC×I2L clipopt ID (SEQ ID NO 434)

### Results:

TABLE 31
EC50 values in pM and gaps of naïve
GSU cells versus knock-out GSU cells

| | EC50 GSU | | EC50 | | EC50 GSU |
|---|---|---|---|---|---|
| | KO MSLN | fold | GSU wt | fold | KO CDH3 |
| | [pM] | gap | [pM] | gap | [pM] |
| MSLN-CDH3 T-cell | 3243.2 | 2429 | 1.34 | 383 | 511.8 |
| engager molecule 1 | | | | | |
| MSLN-CDH3 T-cell | 244.7 | 699 | 0.35 | 215 | 75.2 |
| engager molecule 2 | | | | | |

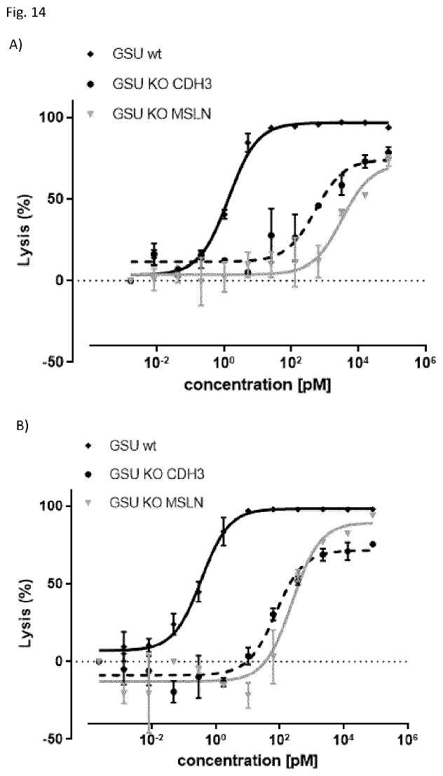

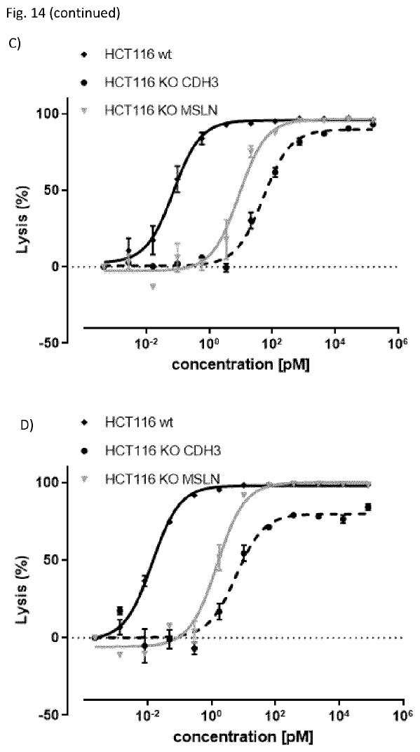

The tested MSLN-CDH3 T-cell engager molecules 1&2 showed increased activity (lower EC50 values) on MSLN and CDH3 double positive GSU wt cells compared to respective GSU k.o cells (GSU CDH3 k.o and GSU MSLN k.o.). The MSLN-CDH3 T-cell engager molecules 1&2 showed EC50 gaps greater 100-fold on MSLN and CDH3 double positive GSU wt cells versus the respective GSU k.o cells (GSU CDH3 k.o and GSU MSLN k.o.) (FIG. 14A, B) and Table 31).

| | EC50 [pM] |
|---|---|
| MSLN-CDH3 T-cell engager | 0.07 |
| molecule 1 on HCT 116 wt | |
| MSLN-CDH3 T-cell engager | 51.8 |
| molecule 1 on HCT 116 KO CDH3 | |
| MSLN-CDH3 T-cell engager | 8.7 |
| molecule 1 on HCT 116 KO MSLN | |

| | EC50 [pM] |
|---|---|
| MSLN-CDH3 T-cell engager | 0.01 |
| molecule 2 on HCT 116 wt | |
| MSLN-CDH3 T-cell engager | 5.9 |
| molecule 2 on HCT 116 KO CDH3 | |
| MSLN-CDH3 T-cell engager | 1.5 |
| molecule 2 on HCT 116 KO MSLN | |

### Legend:

MSLN-CDH3 T-cell engager molecule 1: CH3 15-E11 CC×I2L×G4×scFc xG4×MS 15-B12 CC×I2L_(SEQ ID NO 251) MSLN-CDH3 T-cell engager molecule 2: CH3 15-E11 VAG CC×I2L×G4×scFc×MS 15-B12 CC×I2L clipopt ID (SEQ ID NO 434)

### Results:

TABLE 32
EC50 values in pM and gaps of naïve
HCT 116 cells versus knock-out HCT 116 cells

| | EC50 | | | | EC50 |
|---|---|---|---|---|---|
| | HCT | | EC50 | | HCT |
| | 116 KO | | HCT | | 116 KO |
| | MSLN | fold | 116 wt | fold | CDH3 |
| | [pM] | gap | [pM] | gap | [pM] |
| MSLN-CDH3 T-cell | 8.7 | 122 | 0.07 | 727 | 51.8 |
| engager molecule 1 | | | | | |
| MSLN-CDH3 T-cell | \hat{ } 1.5 | 150 | 0.01 | 590 | 5.9 |
| engager molecule 2 | | | | | |

The tested MSLN-CDH3 T-cell engager molecules 1&2 showed increased activity (lower EC50 values) on MSLN and CDH3 double positive HCT 116 wt cells compared to respective HCT 116 k.o cells (HCT 116 CDH3 k.o and HCT 116 MSLN k.o.). The MSLN-CDH3 T-cell engager molecules 1&2 showed EC50 gaps greater 100-fold on MSLN and CDH3 double positive HCT 116 wt cells versus the respective HCT 116 k.o cells (HCT 116 CDH3 k.o and HCT 116 MSLN k.o.) (FIG. 14C, D) and Table 32).

### Example 13 Hydroxylation Analysis

### Proteolytic Digestion of MSLN-CDH3 T-Cell Engager Molecules 1 and 2

Proteolytic digestions were performed on a filter unit using trypsin (1:20 enzyme/substrate ratio, Roche, #03708969001) and human neutrophile elastase (HNE, 1:20 enzyme/substrate ratio, Elastin Products Co., #SE563) at pH 7.8 (37° ° C., trypsin: 1 h, HNE: 30 min). Prior to digestion the protein was denatured (6M guanidine, pH 8.3), reduced (DTT) and alkylated (Sodium Iodoacetate). The proteolysis was quenched with 8M guanidine (pH 4.7).

### LC-MS/MS Measurement and Data Evaluation

For LC-MS analysis an Agilent 1290 HPLC system connected to a Thermo Scientific™ Q Exactive™ BioPharma platform with an electrospray ion source was used. Separation was performed using a C18 reversed-phase column and gradient elution with mobile phases A (0.1% HCOOH in water) and B (0.1% HCOOH in 90% acetonitrile) at a flow rate of 0.25 ml/min. MS data were produced using full scan positive mode. Additionally, tandem mass spectrometry (MS/MS) data were generated of the most intense ions. Data evaluation and peptide identification was automated using an in-house developed software program.

### MS/MS Analysis

The tryptic peptide Q³⁹-K⁵⁶ was used to calculate the relative abundance of hydroxylation at position K⁵⁶ in MSLN-CDH3 T-cell enganger molecule 1. The MS area of the hydroxylated peptide Q³⁹-K(Hyl)⁵⁶ (charge state 2+ and 3+) from MSLN-CDH3 T-cell enganger molecule 1 was set as numerator and the sum of unmodified peptide Q³⁹-K⁵⁶ (charge state 2+ and 3+) and hydroxylated peptide Q³⁹-K(Hyl)⁵⁶ (charge state 2+ and 3+) was set as denominator. The y- and b-ion series of tryptic peptides Q³⁹-K⁵⁶, Q³⁹-K(Hyl)⁵⁶ and Q³⁹-K⁶³ from MSLN-CDH3 T-cell enganger molecule 1 and 2 were used for MS/MS verification of the modified and unmodified peptide.

TABLE 33
Relative quantification of hydroxylation at
position K56 in MSLN-CDH3 T-cell engager
molecule 1

| | Re- | | | Theo- | | |
|---|---|---|---|---|---|---|
| | ten- | | | ret- | Ob- | |
| | tion | | | ical | served | |
| Pep- | Time | Charge | | Mass | Mass | MS |
| tide | [min] | State | m/z | [Da] | [Da] | Area |
| QAPG | 48.214 | 2 | 1012.480 | 2021.934 | 2021.935 | 9.09 |
| QC*L | | | | | | e+06 |
| EWMG | | | | | | |
| NIAY | | | | | | |
| GVK | | | | | | |
| QAPG | 48.212 | 3 | 675.320 | | 2021.937 | 2.22+ |
| QC*L | | | | | | 07 |
| EWMG | | | | | | |
| NIAY | | | | | | |
| GVK | | | | | | |
| QAPG | 47.487 | 2 | 1020.470 | 2037.929 | 2037.929 | 1.87 |
| QC*L | | | | | | e+06 |
| EWMG | | | | | | |
| NIAY | | | | | | |
| GVK | | | | | | |
| (H | | | | | | |
| yl) | | | | | | |
| QAPG | 47.487 | 3 | 680.652 | | 2037.931 | 4.63+ |
| QC*L | | | | | | 06 |
| EWMG | | | | | | |
| NIAY | | | | | | |
| GVK | | | | | | |
| (H | | | | | | |
| yl) | | | | | | |

Relative Abundance 17.20%
*carboxymaethylated cysteine: +58.005 Da

TABLE 34
Q³⁹-K⁶³ peptide in MSLN-CDH3 T-cell engager
molecule 2

| | Re- | | | Theo- | | |
|---|---|---|---|---|---|---|
| | ten- | | | ret- | Ob- | |
| | tion | | | ical | served | |
| Pep | Time | Charge | | Mass | Mass | MS |
| tide | [min] | State | m/z | [Da] | [Da] | Area |
| QAPG | 50.930 | 2 | 1386.630 | 2770.25 | 2770.256 | 3.35 |
| QC*L | | | | | | e+06 |
| EWMG | | | | | | |
| NIAY | | | | | | |
| GVAG | | | | | | |
| TNYN | | | | | | |
| QK | | | | | | |
| QAPG | 50.930 | 3 | 924.759 | | 2770.250 | 3.73 |
| QC*L | | | | | | e+07 |
| EWMG | | | | | | |
| NIAY | | | | | | |
| GVAG | | | | | | |
| TNYN | | | | | | |
| QK | | | | | | |

*carboxymaethylated cysteine: +58.005 Da

### Ion Exchange Chromatography of T-Cell Engager Molecules 1 and 2

For CEX-HPLC analysis an Agilent 1290 HPLC was used. Separation was performed using a cation exchange chromatography column (YMC Co., Ltd., SF00S05-1046WP) and gradient elution with mobile phases A (Thermo Scientific, 085346) and B (Thermo Scientific, 085348) at a flow rate of 1.00 ml/min.

### Results:

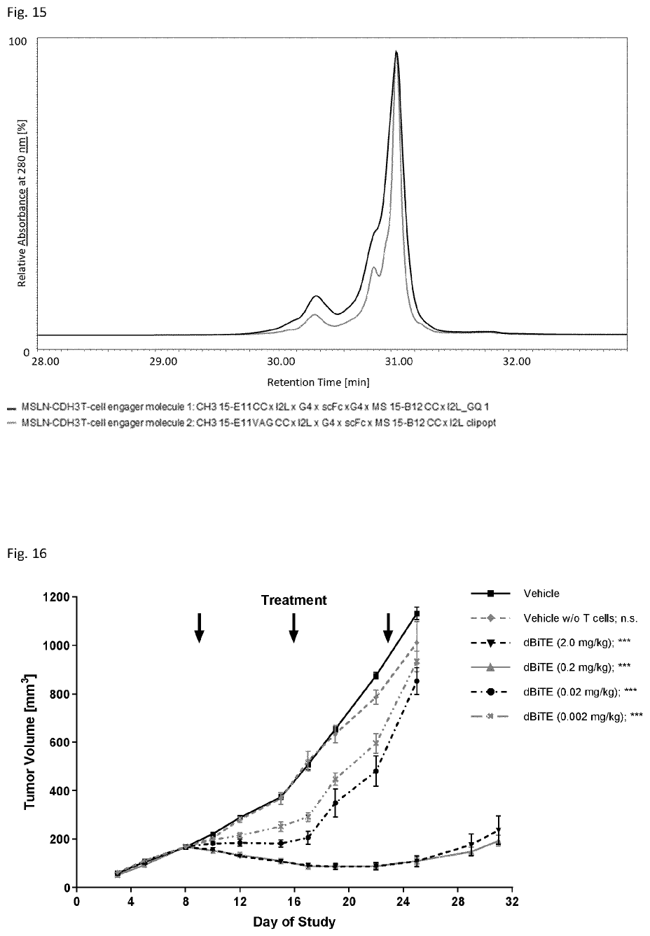

Hydroxylation at position K56 (relative abundance 17.20%, see Table 33) was observed in MSLN-CDH3 T-cell enganger molecule 1. Replacing lysine (K) at position 56 to alanine (A), no hydroxylation at position A56 was observed in MSLN-CDH3 T-cell enganger molecule 2 (see Table 34). Using CEX-HPLC analysis, the resulting CEX main peak heterogeneity of MSLN-CDH3 T-cell enganger molecule 2 was decreased compared to MSLN-CDH3 T-cell enganger molecule 1 (see FIG. 15).

### Example 14: Physicochemical Property Analysis of Molecules of the Invention

### Isolation and Formulation of Monomeric Dual-Targeting Antigen-Binding Molecules and Determination of Protein Yield

Cell culture supernatant (SN) containing expressed dual-targeting antigen-binding molecules was clarified by centrifugation and filtrated by using a 0.2 μM filtration step.

Monomeric protein was isolated by applying a two-step purification process on an Äkta Pure 25 system (Cytiva, Freiburg im Breisgau, Germany) generating a selected liquid volume of monomeric dual-targeting antigen-binding molecule followed by formulation and concentration adjustment of this volume.

TABLE 35
Expression yields of monomeric dual-targeting antigen-
binding molecules in a two-step purification process

| | Monomer yield |
|---|---|
| Dual-targeting antigen-binding molecule | [mg/L SN] |
| CH3 15-E11 CC × I2L × G4 × scfc × G4 × MS | 40.6 |
| 15-B12 CC × I2L_GQ | |
| SEQ ID NO 251 | |
| CH3 15-E11_1_VAG_CC × I2L × G4 × scFc × | 12.2 |
| G4 × MS 15-B12 CC × I2L clipopt_DI | |
| SEQ ID NO 434 | |

Expression yields of dual-targeting antigen-binding molecules

### Evaluation of Dual-Targeting Antigen-Binding Molecule Surface Hydrophobicity

Isolated and formulated dual-targeting antigen-binding molecule monomer adjusted to a defined protein concentration was transferred into autosampler fitting sample vials and measured on an Äkta Purifier 10 FPLC system (Cytiva, Freiburg im Breisgau, Germany). A Hydrophobic Interaction Chromatography HIC column was equilibrated with formulation buffer and a defined volume of protein solution applied at a constant formulation buffer flow. Detection was done by OD280 nm optical absorption. Elution behavior was determined by peak shape respectively mathematically calculation of declining signal peak slope. Steeper slope/higher slope values indicate less hydrophobic interaction of the protein surface compared to constructs with more flat elution behavior and lower slope value.

TABLE 36
HIC elution slopes of dual-targeting antigen-binding molecule

| | HIC |
|---|---|
| Dual-targeting antigen-binding molecule | elution slope |
| CH3 15-E11 CC × I2L × G4 × scfc × G4 × MS 15-B12 | 40.69 |
| CC × I2L_GQ | |
| SEQ ID NO 251 | |
| CH3 15-E11_1_VAG_CC × I2L × G4 × scFc × G4 × | 46.43 |
| MS 15-B12 CC × I2L clipopt_DI | |
| SEQ ID NO 434 | |

Peak slope of analyzed dual-targeting antigen-binding molecule after injection on a HIC column

### Evaluation of Dual-Targeting Antigen-Binding Molecule Aggregation Temperature

Isolated and formulated dual-targeting antigen-binding molecule monomer adjusted to a defined protein concentration was pipetted in doubles into a 96 well plate and overlaid with paraffin oil. The 96 well plate was transferred to a dynamic light scattering DLS reader (DynaPro Plate Reader II, Wyatt, Dernbach, Germany) capable of heating the plate at a defined rate in a fixed temperature range. Measurement was performed from 40° ° C. to 70° C. at a defined rate of temperature increase. Detection was done by dynamic light scattering determining the hydrodynamic radius of the constructs over the temperature ramp. Temperature at begin of increase of hydrodynamic radius was defined as aggregation temperature.

TABLE 37
DLS aggregation temperature of dual-
targeting antigen-binding molecules

| | Aggregation |
|---|---|
| Dual-targeting antigen-binding molecule | temperature [° C.] |
| CH3 15-E11 CC × I2L × G4 × scfc × G4 × | 58.98 |
| MS 15-B12 CC × I2L_GQ | |
| SEQ ID NO 251 | |
| CH3 15-E11_1_VAG_CC × I2L × G4 × scFc × | 60.42 |
| G4 × MS 15-B12 CC × I2L clipopt_DI | |
| SQ ID NO 434 | |

DLS aggregation temperature of dual-targeting antigen-binding molecules

### Evaluation of Dual-Targeting Antigen-Binding Molecule Long Term Storage Stability

Isolated and formulated dual-targeting antigen-binding molecule monomer adjusted to a defined protein concentration was aliquoted and stored at 37° C. for one week in a temperature-controlled incubator.

An analytical SEC column of 15 cm length was connected to an UPLC system (Aquity, Waters, Eschborn, Germany) and equilibrated with a suitable elution buffer. A volume of 10 μl treated dual-targeting antigen-binding molecule monomer solution was injected under a constant flow of elution buffer while detecting optical absorbance at 210 nm wavelength until all protein and formulation constituents were eluted from the column.

The same procedure was performed for an untreated sample as reference.

Monomer percentage was calculated by comparing the area of the monomeric main peak to the area of all protein peaks detected.

TABLE 38
Monomer percentage of dual-targeting antigen-binding
molecules after one-week storage at 37° C.

| | Monomer |
|---|---|
| Dual-targeting antigen-binding molecule | decrease [%] |
| CH3 15-E11 CC × I2L × G4 × scfc × G4 × | 0.88 |
| MS 15-B12 CC × I2L_GQ | |
| SEQ ID NO 251 | |
| CH3 15-E11_1_VAG_CC × I2L × G4 × scFc × G4 × | 1.72 |
| MS 15-B12 CC × I2L clipopt_DI | |
| SEQ ID NO 434 | |

Monomer percentage of dual-targeting antigen-binding molecules

### Evaluation of Dual-Targeting Antigen-Binding Molecule Freeze Thaw Stability

Isolated and formulated dual-targeting antigen-binding molecule monomer adjusted to a defined protein concentration was aliquoted and frozen/thawed at −80° ° C./room temperature three times for 30 min. for each step.

An analytical SEC Column of 15 cm length was connected to an UPLC system (Aquity, Waters, Eschborn, Germany) and equilibrated with a suitable elution buffer. A volume of treated 10 μl dual-targeting antigen-binding molecule monomer solution was injected under a constant flow of elution buffer while detecting optical absorbance at 210 nm wavelength until all protein and formulation constituents were eluted from the column.

Monomer percentage was calculated by comparing the area of the monomeric main peak to the area of all protein peaks detected.

TABLE 39
Monomer percentage of dual-targeting antigen-binding
molecules after three freeze/thaw cycles

| | Monomer |
|---|---|
| Dual-targeting antigen-binding molecule | percentage [%] |
| CH3 15-E11 CC × I2L × G4 × scfc × G4 × | 99.52 |
| MS 15-B12 CC × I2L GQ | |
| SEQ ID NO 251) | |
| CH3 15-E11_1_VAG_CC × I2L × G4 × scFc × G4 × | 99.12 |
| MS 15-B12 CC × I2L clipopt_DI | |
| SEQ ID NO 434 | |

Monomer percentage of dual-targeting antigen-binding molecules after three freeze/thaw cycles

### Determination of Dual-Targeting Antigen-Binding Molecule Charge Heterogeneity

An analytical cation exchange column was connected to an UPLC system (Aquity, Waters, Eschborn, Germany) and equilibrated with a low conductivity equilibration/binding buffer=Buffer_A. A second buffer system with high conductivity suitable for protein elution was also connected to the UPLC system=Buffer_B.

Detection for the analytical procedure was set to 280 nm optical wavelength.

A volume of 10 μl dual-targeting antigen-binding molecule monomer solution was injected under a constant flow of Buffer_A buffer.

After protein binding and washing out of formulation buffer constituents a gradient of Buffer_B was applied at the same flow rate with a linear increase from 0% to 100% Buffer_B.

Main peak percentage was calculated by comparing the area of the main peak to the area of all protein peaks detected.

TABLE 40
Main peak percentage of dual-targeting antigen-binding
molecules in analytical cation exchange chromatography

| | Monomer |
|---|---|
| Dual-targeting antigen-binding molecule | percentage [%] |
| CH3 15-E11 CC × I2L × G4 × scfc × G4 × | 87.71 |
| MS 15-B12 CC × I2L_GQ | |
| SEQ ID NO 251 | |
| CH3 15-E11_1_VAG_CC × I2L × G4 × scFc × G4 × | 78.30 |
| MS 15-B12 CC × I2L clipopt_DI | |
| SEQ ID NO 434 | |

Main peak percentage of dual-targeting antigen-binding molecules in analytical cation exchange chromatography

### Example 15: Evaluation of CDH3 MSLN Dual Targeting Antigen-Binding Molecules In Vitro Affinity

Cell-based affinity of CDH3 MSLN dual targeting antigen-binding molecules was determined by nonlinear regression (one site—specific binding) analysis. CHO cells expressing human CDH3, cyno CDH3, human MSLN or cyno MSLN were incubated with decreasing concentrations of CDH3 MSLN dual targeting antigen-binding molecules (12.5 nM on CDH3 cell lines, 800 nM on MSLN cell lines, step 1:2, 11 steps) for 16 h at 4° ° C. Bound CDH3 MSLN dual targeting antigen-binding molecules were detected with Alexa Fluor 488-conjugated AffiniPure Fab Fragment Goat Anti-Human IgG (H+L). Fixed cells were stained with DRAQ5, Far-Red Fluorescent Live-Cell Permeant DNA Dye and signals were detected by fluorescence cytometry. Respective equilibrium dissociation constant (Kd) values were calculated with the one site—specific binding evaluation tool of the GraphPad Prism software. Mean Kd values and affinity gaps were calculated with Microsoft Excel.

TABLE 41
Cell-based affinities of CDH3 MSLN dual targeting antigen-binding molecules

| | Cell based | Cell based | Affinity | Cell based | Cell based | Affinity |
|---|---|---|---|---|---|---|
| | affinity hu | affinity cy | gap | affinity hu | affinity cy | gap |
| | MSLN (Kd) | MSLN (Kd) | Kd_{cy}/Kdₕᵤ | CDH3 (Kd) | CDH3 (Kd) | Kd_{cy}/Kdₕᵤ |
| Molecule | [nM] | [nM] | MSLN | [nM] | [nM] | CDH3 |
| Dual targeting antigen- | 33.78 ± 11.59 | 51.42 ± 9.25 | 1.52 | 0.16 ± 0.04 | 0.19 ± 0.07 | 1.20 |
| binding molecule 1 | | | | | | |
| Dual targeting antigen- | 39.96 ± 38.16 | 50.29 ± 15.77 | 1.26 | 0.51 ± 0.021 | 0.57 ± 0.10 | 1.12 |
| binding molecule 2 | | | | | | |

Cell-based affinities of CDH3 MSLN dual targeting antigen-binding molecules on target-transfected CHO cells were determined by nonlinear regression (one site - specific binding) analysis. Mean Kd values were calculated from three independent measurements. Affinity gaps were determined by dividing the cyno Kd by the human Kd.

### Result

Cell-based affinity measurements revealed, that CDH3 MSLN dual targeting antigen-binding molecules 1 and 2 have comparable affinities on target-transfected CHO cells expressing human CDH3, cyno CDH3, human MSLN or cyno MSLN. Affinity gaps of both molecules are comparable as well.

### Legend

CDH3 MSLN dual targeting antigen-binding molecule 1: CH3 15-E11 CC×I2L×G4×scFc×G4×MS 15-B12 CC×I2L_

CDH3 MSLN dual targeting antigen-binding molecule 2: CH3 15-E11 VAG CC×I2L×G4×scFc×MS 15-B12 CC×I2L clipopt

### Example 16: In Vivo Efficacy Testing of CDH3×MSLN Bispecific Antigen-Binding Molecule

The therapeutic efficacy in terms of anti-tumor activity was assessed in an advanced stage human tumor xenograft model. On day 1 of the study, 5×10⁶ cells of a human target cell antigen (CDH3×MSLN) positive cancer cell line are subcutaneously injected in the right dorsal flank of female NOD/SCID mice. When the mean tumor volume reaches about 100 mm3, in vitro expanded human CD3 positive T cells are transplanted into the mice by injection of about 2×107 cells into the peritoneal cavity of the animals. Mice of vehicle control group 1 do not receive effector cells and are used as an un-transplanted control for comparison with vehicle control group 2 (receiving effector cells) to monitor the impact of T cells alone on tumor growth. The treatment with CDH3×MSLN bispecific antigen-binding molecule of SEQ ID NO 251 starts when the mean tumor volume reaches about 200 mm3. The mean tumor size of each treatment group on the day of treatment start should not be statistically different from any other group (analysis of variance). Mice are treated with 0.5 mg/kg/day of CHD3×MSLN bispecific antigen-binding molecule by intravenous bolus injection on days of study 9, 16 and 24. Tumors are measured by caliper during the study and progress evaluated by intergroup comparison of tumor volumes (TV). The tumor growth inhibition T/C [%] is determined by calculating TV as T/C %=100×(median TV of analyzed group)/(median TV of control group 2). As it is evident from FIG. 16, treatment with 0.2 mg/kg or 2 mg/kg CHD3×MSLN bispecific antigen-binding molecule effectively inhibited tumor growth in vivo.

### Example 17: Modelling of Multitargeting Bispecific Antigen-Binding Molecules According to the Invention

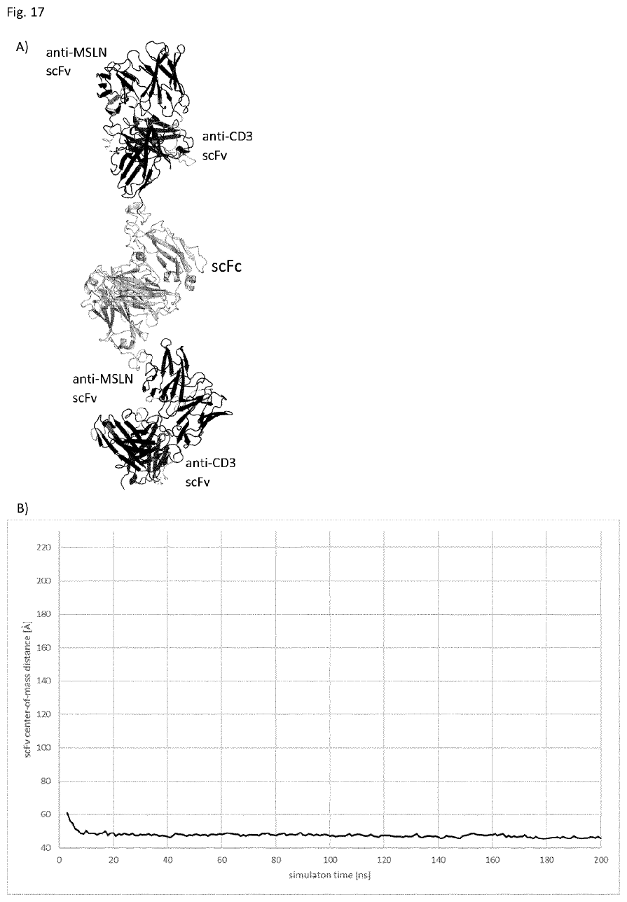

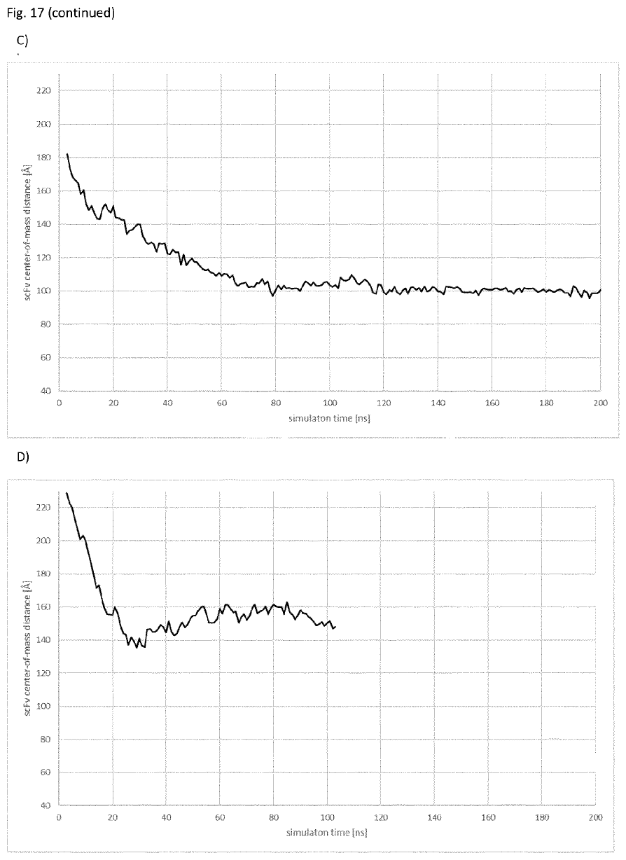

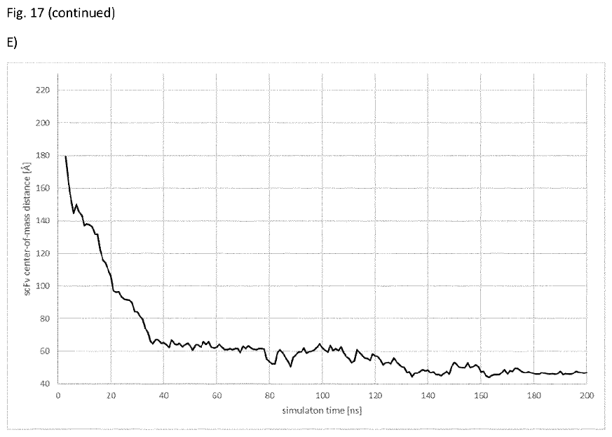

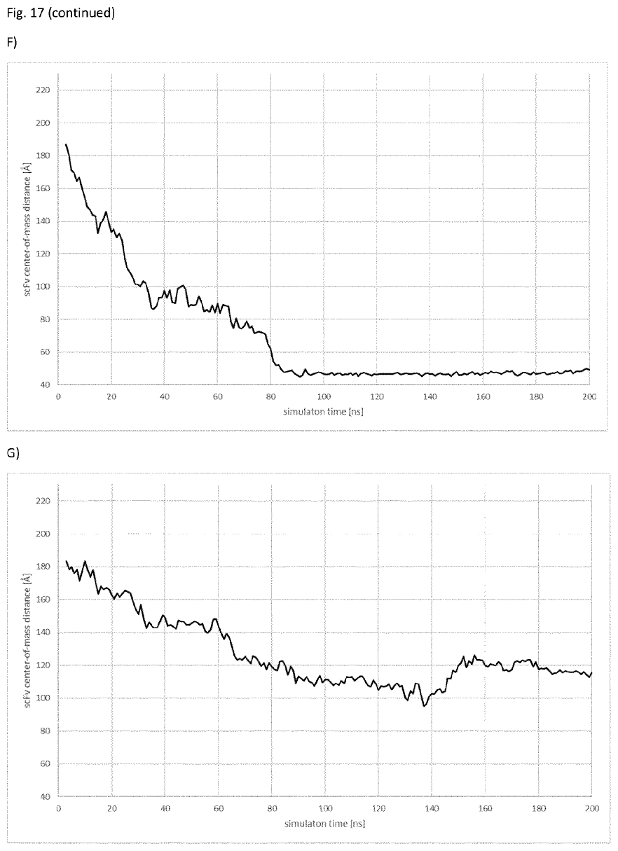

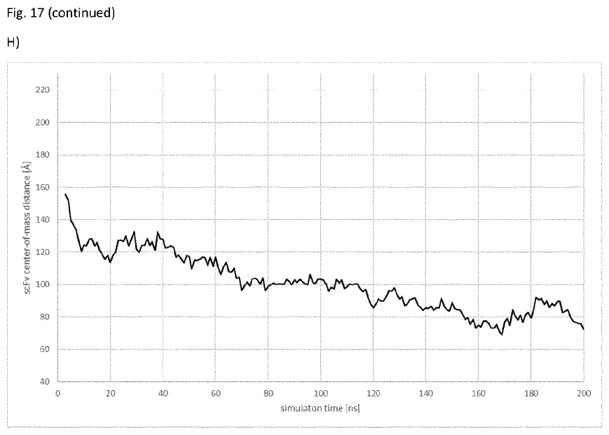

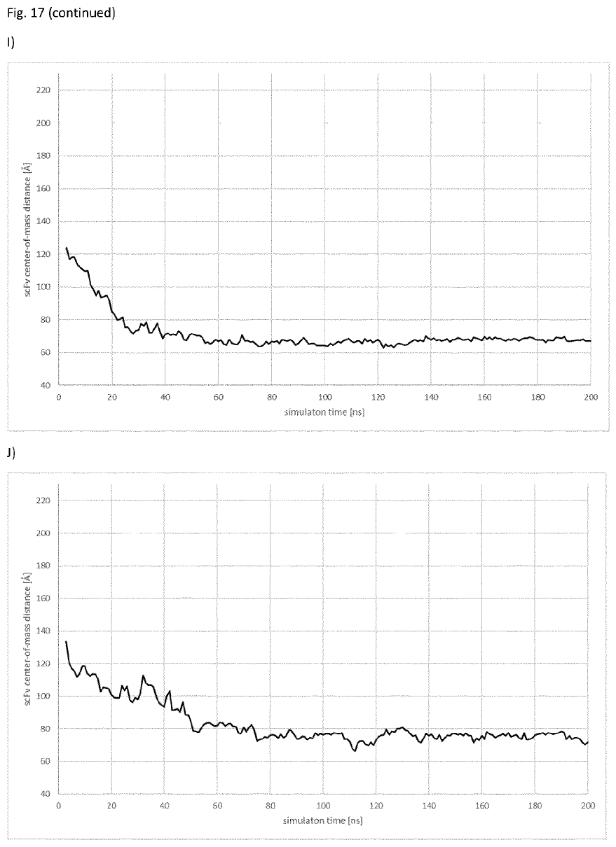

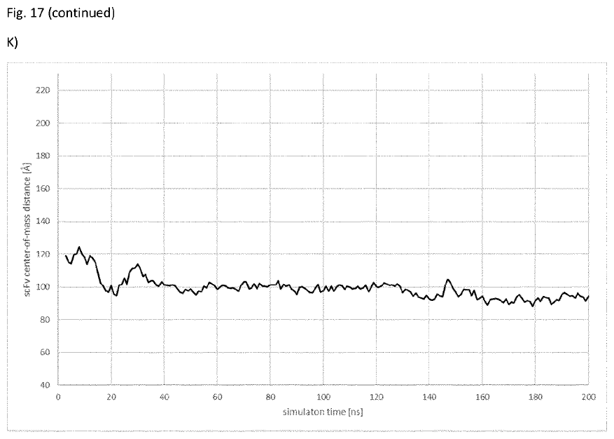

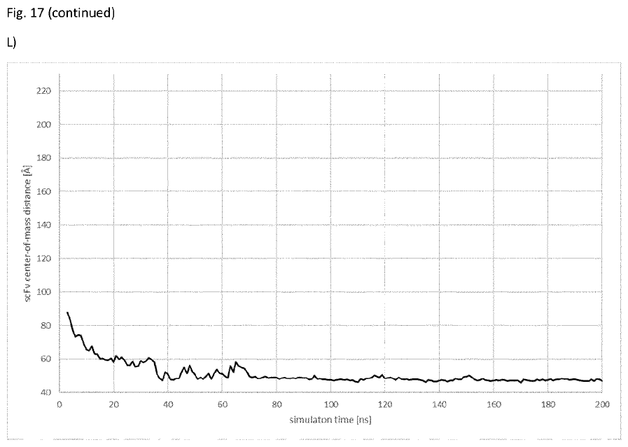

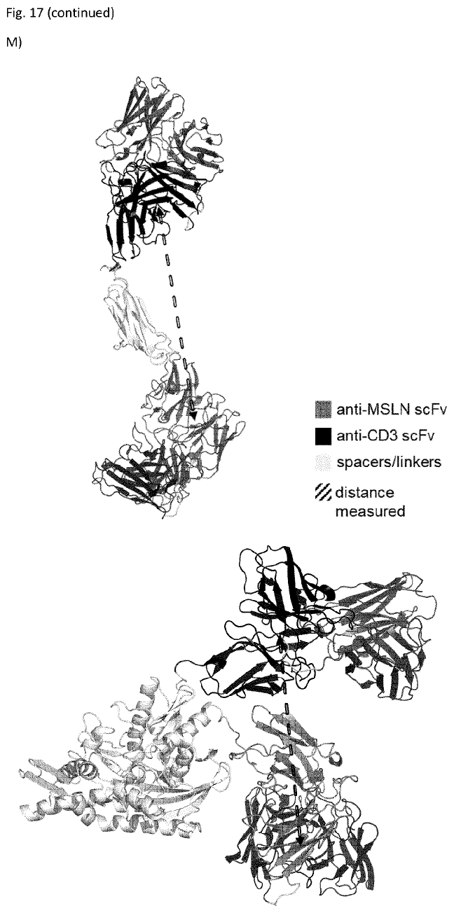

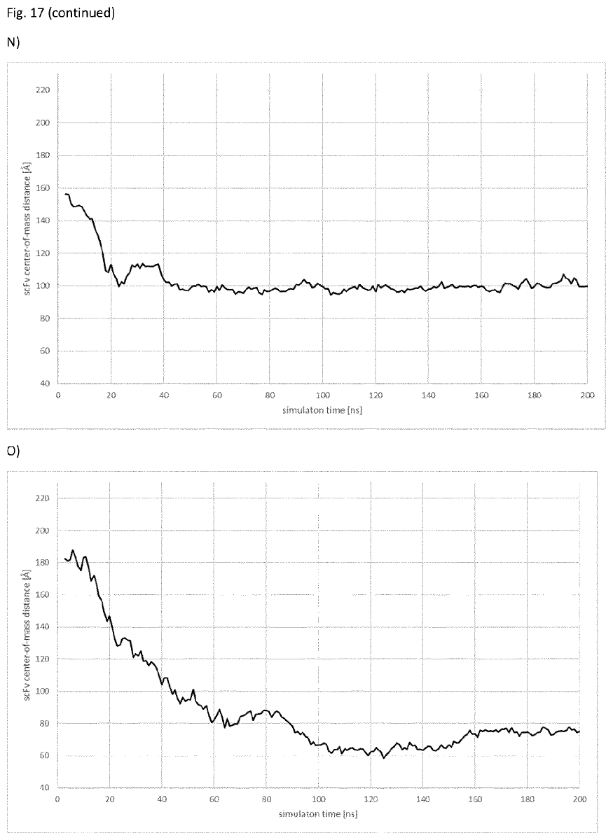

Surrogates of multitargeting bispecific antigen-binding molecules of the invention were modelled to measure the inter-domain distances for various linker/spacer sizes (3D model depiction FIG. 17A). Starting molecular models were based on internal structural data of a canonical anti-MSLN molecule comprised of an MSLN-binding scFv and a CD3-binding I2C scFv. Due to providing the highest homology and completeness among available internal and public crystal data, this structure was used to represent both the N- and C-terminal molecule entities in all surrogate models. Missing residues and linkers were added using the Schrodinger software suite (version 2020-4, Schrödinger, NY, US). Similarly, the “spacer” groups of interest (scFc (FIG. 17C), PD1 (FIG. 17H), HSA (FIG. 17G), ubiquitin (FIG. 17I), SAND (FIG. 17J), Beta-2-microglobulin (FIG. 17K) and HSP70-1 (FIG. 17L) were modeled with the Schrodinger suite based on closest public PDB structures (PDB codes 1HZH, 6JJP, and 5VNW respectively) and cross-linked with the 2 molecule copies. Measurements and images were generated with PyMOL (version 2.3.3, Schrödinger, NY, US). The general approach of MD has been explained in the general description of the invention.

TABLE 42
Median and maximum distance conferred by respective
spacers between bispecific entities

| | median distance | max distance |
|---|---|---|
| spacer | (scFv COM) | (scFv COM) |
| G4S | 47 | 61 |
| scFc | 101 | 182 |
| 2 × scFc | 153 | 229 |
| (G4S)10 | 48 | 179 |
| (EAAAK)10 | 47 | 187 |
| HAS | 114 | 183 |
| PD1 | 86 | 156 |

All spacer lengths (i.e. number of GGGGS monomer repeats) were based on sequences of experimentally-tested molecules. Each homology model was built in an extended conformation, maximizing the center-of-mass (COM) distance between the N-terminal I2C (CD3 binder) and C-terminal MSLN-binder (target binder). Hence, the starting molecule conformations are indicative of the maximum COM distance each molecule could theoretically achieve. To probe the stability of these conformations, each model was subjected to a 200 nanosecond (100 ns in case of the double scFc spacer due very slow simulations speed) explicit-solvent MD simulation with Desmond, a component of the Schrodinger suite. A general observation for all 11 simulated systems (respective spacers: G4S, scFc, 2×scFc, (G4S)10, (EAAAK)10, HSA, PD1, ubiquitin, SAND, Beta-2microblobulin, HSP70-1) was a reduction of the inter-scFv COM distance indicating that the extended conformations are only (if at all) possible in presence of the targets (the N- and C-terminal antigen-binding molecule structures remained largely unchanged due to corresponding to a stable crystal structure conformation). For the large spacers with defined secondary structures (scFc, 2×scFc (FIG. 17D), HSA, PD1) the distance reduction was small to moderate and the scFv moieties remained clearly separated at the end of each simulation (median COM distances upon discarding the first half of each simulation: 101, 153, 114, and 86 Å respectively). The flexible (G4S)10 and (EAAAK)10 linkers “collapsed” into more compact conformations, bringing the scFv moieties much closer together (median COM distance of 48 and 47 Å). Out of these 2 linkers, (EAAAK)10 led to a slightly more stable conformation which might be associated with higher selectivity. The short G4S linker was unable to keep the scFv moieties apart and they were seen to strongly interact throughout the entire simulation (median COM distance of 47 Å but with the VH CDR3 loops much closer to each other than in any other system). Ubiquitin as a spacer of 73 aa maintained Center-of-mass median distance between 1^{st} CD3 scFv and 2^{nd} MSLN scFv of 67 Å, meaning effective separation. SAND as a spacer of 89 aa maintained Center-of-mass median distance between 1^{st} CD3 scFv and 2^{nd} MSLN scFv of 77 Å. Beta-2-microglobulin as a spacer of 97 aa maintained Center-of-mass median distance between 1st CD3 scFv and 2^{nd} MSLN scFv of 95 Å, i.e. comparable to preferred scFc. In contrast, HSP70-1 as a spacer of 378 aa did maintained Center-of-mass median distance between 1^{st} CD3 scFv and 2^{nd} MSLN scFv of only 48 Å indicating insufficient separation of the two bispecific entities. The simulation of the molecule with beta-2-microglobulin (FIG. 17M left) and HSP70-1 (FIG. 17M right) is visualized by respective representative structures which indicate presence and absence of separation by the spacer, respectively.

Like shown above for a molecule with two MSLN target binders, good separation and scFv mobility by scFc (SEQ ID NO: 25) as spacer in the context of the present invention was observed for MSLN and FOLR1 as target binders showing center-of-mass median distance between 1^{st} CD3 scFv and FolR1 scFv of 99 Å (FIG. 17N) and for MSLN and CDH19 as target binders showing center-of-mass median distance between 1^{st} CD3 scFv and CDH19 scFv: 76 Å (FIG. 17O).

### Example 18 Comparative Clinical Safety Study of Multitargeting Bispecific Antigen-Binding Molecule According to the Invention in Cynomolgus Monkey

A monotargeting mesothelin (MSLN)-targeting bispecific antigen-binding molecule (molecule 1, SEQ ID NO 1183) that showed in vitro efficacy was evaluated in a repeat-dose toxicology study in cynomolgus monkey, a pharmacologically relevant species. Molecule 1 was administered at doses of 0.1, 1.5, 5/1.5, or 15 g/kg by 30-minute intravenous infusion (three animals/sex/group) once weekly for 4 weeks (i.e. administered on Days 1, 8, 15, and 22). Animals from the 5/1.5 μg/kg group received 5 μg/kg on Day 1 and 1.5 μg/kg from Day 8 onwards. Scheduled necropsy was conducted at the end of the dosing phase on Day 29, or after a 4-week recovery period on Day 57. Molecule 1-related clinical and anatomic pathologic changes were generally similar between unscheduled (Days 3, 4, or 8) and scheduled (Days 29, 57) euthanasia cohorts, albeit with an increase in incidence and severity in unscheduled euthanasia individuals.

Molecule 1 showed dose-limiting toxicity with widespread tissue effects in vivo. Doses of 1.5 μg/kg, 5 μg/kg and 15 μg/kg were not tolerated. A single male animal at 1.5 μg/kg. 3 males and 2 females at 5 μg/kg, and all animals at 15 μg/kg were euthanized for humane reasons on Day 3 or 4. In addition, one female animal that received one dose at 5 μg/kg on Day 1 followed by a single dose at 1.5 μg/kg on Day 8 was euthanized on Day 8 due to declining clinical condition. These animals had severe clinical signs, which included dehydration, decreased activity, decreased food consumption, and hunched appearance. Additional Molecule 1-related clinical signs in scheduled euthanasia animals included lack of feces, vomitus material, reduced appetite and decrease in mean body weights. Molecule 1-induced pharmacological effects indicative of the bispecific T cell engager mode of action, such as (but not limited to) an acute phase response (typified by elevated C-reactive protein), transient cytokine release and changes in activation of circulating lymphocytes.

At ≥1.5 μg/kg, administration of molecule 1 resulted in multi-organ inflammation involving mesothelin-expressing tissues/cell types including mesothelial cell-lined serosal surfaces of abdominal and thoracic viscera and epithelium of several tissues, often involving basilar layers. Inflammation and fibroplasia/fibrosis associated with mesothelial cell-lined serosal surfaces culminated in formation of visceral adhesions in some animals. Adhesions were apparent macroscopically in the liver and heart (pericardium) in a few animals treated at 1.5 μg/kg on Day 29, but serosal fibroplasia was more widespread microscopically. Tissues that demonstrated serosal or capsular changes on organ surfaces included the following (all fates): kidney, liver, heart, spleen, lung, stomach, duodenum, jejunum, ileum, cecum, colon, rectum, mesentery, urinary bladder, ovary, cervix, and uterus. Tissues that demonstrated epithelial changes included the following (all fates): kidney, urinary bladder, esophagus, cervix, epididymis, conjunctiva, mammary gland, mandibular salivary gland, seminal vesicle, skin, duodenum, stomach, tongue, tonsil, trachea, uterus, and vagina. Mesothelial and epithelial-related changes were associated with secondary reactive tissue changes including epithelial degeneration/necrosis, erosion/ulceration, regeneration, edema with fibrin exudation and/or hemorrhage. Glomerular changes were associated with mild increased glomerular mesangium. Clinical pathology changes were consistent with both systemic and tissue specific inflammatory responses.

Light microscopic changes partially recovered in several tissues at 0.10 and 5/1.5 μg/kg after 4 weeks without treatment; most fibrotic changes were not fully reversible. The highest non-severely toxic dose level (HNSTD) for molecule 1 was determined to be 0.1 μg/kg.

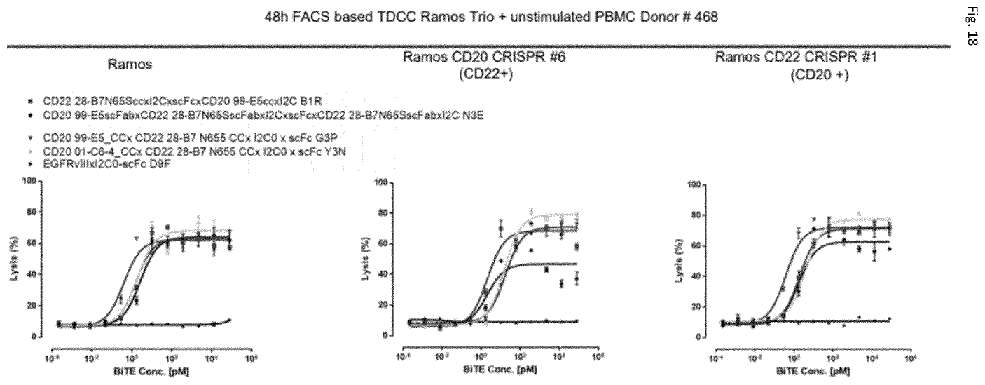

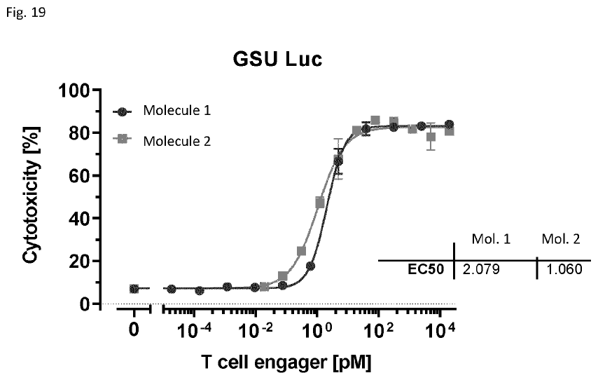

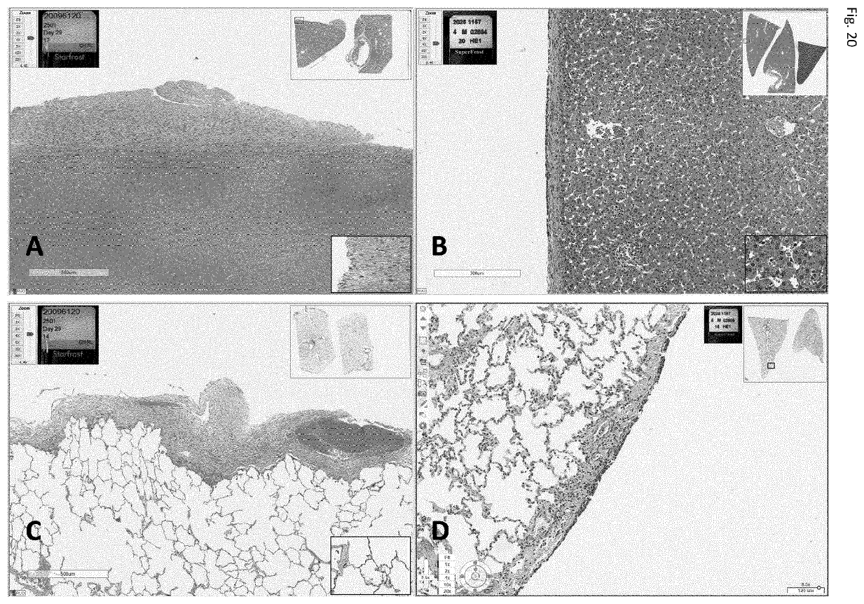

To reduce toxicity, molecule 2 (SEQ ID NO: 251, CH3 15-E11 CC×I2Lopt×G4×scFc SEFL2 clipopt×G4×MS 15-B12 CC×I2L_GQ) was developed as a multitargeting (CDH3-MSLN) bispecific antigen-binding molecule in which also a lower affinity MSLN binder was used. Such lower affinity binder was possible due to the aviditiy effect of the two preferably low affinity binders of the multitargeting bispecific molecule according to the present invention. This molecule 2 is preferably only active if both antitumor targets are bound simultaneously as generally described herein. In vitro efficacy of molecule 2 against human carcinoma cell lines expressing both targets, was comparable to molecule 1. FIG. 19 shows an example cytotoxicity assay in which human T cells were incubated with the human gastric cancer cell line GSU Luc at an ET ratio of 10:1 for 72 hours. The resulting EC₅₀ values were within a similar range (2.078 μM for molecule 1 versus 1.060 μM for molecule 2, respectively, see FIG. 19).

To evaluate whether molecule 2 reduced MSLN-directed toxicity, a repeat-dose toxicology study was conducted in male cynomolgus monkeys via slow IV bolus administration at doses of 1, 10, 100 or 1000 μg/kg on Day 1, 8 and 15. There were no mortalities, and no treatment-related clinical signs, or effects on body weight, food consumption, body temperature, ophthalmoscopic examinations, or coagulation or urinalysis parameters. Similar to molecule 1, molecule 2 induced pharmacological effects indicative of the bispecific T cell engager molecule mode of action, such as (but not limited to) an acute phase response (typified by elevated C-reactive protein), transient cytokine release and changes in activation of circulating lymphocytes.

Administration of molecule 2 at ≥10 μg/kg was associated with light microscopic changes including generally minimal or mild mononuclear or mixed inflammatory cell infiltration of the serosa of multiple organs, associated with focal/multifocal mesothelial hypertrophy. Additional microscopic changes such as mucosal hypertrophy/hyperplasia in the esophagus, mixed cell infiltration in the tongue (with epithelial degeneration of the mucosa) and trachea (with goblet cell hypertrophy), and stress-related atrophy of the thymus were noted in 2 animals dosed at 100 μg/kg or 1000 μg/kg.

In contrast to molecule 1, molecule 2 induced less severe histopathological changes at 1000 μg/kg than molecule 1 at 1.5 μg/kg. FIG. 20 shows representative histopathological hematoxylin/eosin staining of liver (FIG. 20A, B) and lung (FIG. 20C, D) from an animal treated with 1.5 μg/kg molecule 1 (A. C) and an animal treated with 1000 μg/kg molecule 2 (B, D). Whereas molecule 1 had induced marked capsular fibroplasia/fibrosis (A) and formation of interlobar adhesions in the liver at the end of the dosing period on Day 29, molecule 2 only induced minimal multifocal mesothelial hypertrophy and mixed cell infiltration/inflammation of the serosa at the end of the dosing period on Day 16 (B). No adhesions were noted in any animal treated with molecule 2. Similarly, while molecule 1 induced moderate fibroplasia/fibrosis of the lung pleura (D), the lung of the animal treated with molecule 2 did not show fibroplasia/fibrosis (D).

### CONCLUSION

A dose of 1.5 μg/kg molecule 1 was not tolerated and resulted in mortality whereas a dose of 0.1 μg/kg was tolerated. Conversely, molecule 2 was tolerated at doses of up to 1000 μg/kg. Histopathological changes seen with molecule 1 were generally more severe at doses of 1.5 μg/kg than those with molecule 2 at 1000 μg/kg, respectively. Adhesions or irreversible fibrotic changes as induced by molecule 1 were absent after treatment with molecule 2. Therefore, the tolerability of molecule 2 is about 600 (histopathology) to 10.000 (tolerated dose) times higher than for molecule 1, despite equivalent in vitro potency against tumor cells.

### Example 19 Selectivity Gap of Single-Chain Multitargeting Bispecific T-Cell Engager Molecule Vs. Dual-Chain Multitargeting Bispecific T-Cell Engager Molecule

The assays were prepared as in previous cytotoxicity Examples on MSLN-CDH3 T-cell engager molecules of the present invention.

TABLE 43
EC50 values in pM and gaps of naïve
GSU cells versus target knockout GSU cells.

| | EC50 GSU | Fold | | Fold | EC50 GSU |
|---|---|---|---|---|---|
| | KO CDH3 | selectivity | EC50 GSU | selectivity | KO MSLN |
| | [pM] | gap | wt [pM] | gap | [pM] |
| MSLN-CDH3 T-cell | 289 | 138 | 2.1 | 84 | 177 |
| engager molecule 1 | | | | | |
| MSLN-CDH3 T-cell | 391 | 121 | 3.2 | 165 | 533 |
| engager molecule 2 | | | | | |
| MSLN T-cell | 2.1 | 1 | 3.1 | — | b.c.t. |
| engager molecule 1 | | | | | |
| CDH3 T-cell | b.c.t. | — | 201 | 1 | 180 |
| engager molecule 1 | | | | | |

b.c.t: below calculation threshold

The tested MSLN-CDH3 T-cell engager molecules 1 and 2 showed increased activity (lower EC50 values) on MSLN and CDH3 double positive GSU wt cells compared to respective GSU KO cells (GSU KO CDH3 and GSU KO MSLN). These molecules showed EC50 selectivity gaps greater 80-fold on double positive target cells versus single positive target cells. MSLN-CDH3 T-cell engager molecules 1 comprises one multitargeting bispecific T-cell engager polypeptide chain, whereas MSLN-CDH3 T-cell engager molecule 2 comprises two bispecific T-cell engager polypeptide chains which are linked by a heterodimer Fc to build a two-chained multitargeting bispecific T-cell engager molecule. Both have the domain arrangement of [target binding domain×CD3 binding domain×spacer×target binding domain×CD3 binding domain]. Mono targeting control T-cell engager molecules had comparable activity on single positive vs. double positive cells (selectivity gap of ˜1).

Legend

| | |
|---|---|
| MSLN-CDH3 T-cell | MS 15-B12 CC × I2L × G4 × |
| engager molecule 1 | scFc × G4 × CH3 15-E11 CC × I2L |
| | (SEQ ID NO: 1078) |
| MSLN-CDH3 T-cell | MS 15-B12 CC × 6H10.09 × (G4) × |
| engager molecule 2 | heFc (A) * heFc (B) × (G4) × CH3 |
| | 15-E11 CC × 6H10.09 (Seq ID 326 + 327) |
| MSLN T-cell | MSLN 5F11 × I2C -scFc (EQ ID NO: 1183) |
| engager molecule 1 | |
| CDH3 T-cell | CH3 G8A 6-B12 × I2C0-scFc |
| engager molecule 1 | |

Example 20 Selectivity gap of multitargeting bispecific T-cell engager polypeptides (MBiTEP) with varied CD3 affinities The assays were prepared as in previous cytotoxicity Examples on MSLN-CDH3 T-cell engager molecules of the present invention.

TABLE 44
EC50 values and selectivity gaps of naive
GSU cells versus target knockout GSU cells.

| | EC50 GSU | fold | EC50 | fold | EC50 GSU |
|---|---|---|---|---|---|
| | KO CDH3 | selectivity | GSU wt | selectivity | KO MSLN |
| | [pM] | gap | [pM] | gap | [pM] |
| MSLN-CDH3 T-cell | 23.0 | 51 | 0.45 | 118 | 53.3 |
| engager molecule 1 | | | | | |
| MSLN-CDH3 T-cell | 19.6 | 30 | 0.66 | 108 | 71.2 |
| engager molecule 2 | | | | | |
| MSLN-CDH3 T-cell | 1.1 | 3 | 0.35 | 2 | 0.8 |
| engager molecule 3 | | | | | |
| MSLN-CDH3 T-cell | 6.9 | 24 | 0.28 | 84 | 17.7 |
| engager molecule 4 | | | | | |
| MSLN-CDH3 T-cell | 2.0 | 18 | 0.11 | 16 | 1.7 |
| engager molecule 5 | | | | | |
| MSLN-CDH3 T-cell | 2.6 | 58 | 0.04 | 284 | 12.7 |
| engager molecule 6 | | | | | |
| MSLN-CDH3 T-cell | 6.0 | 13 | 0.48 | 22 | 10.6 |
| engager molecule 7 | | | | | |
| MSLN T-cell | 1.0 | 1 | 1.02 | — | b.c.t. |
| engager molecule 1 | | | | | |
| CDH3 T-cell | b.c.t. | — | 70.16 | 1 | 64.5 |
| engager molecule 1 | | | | | |

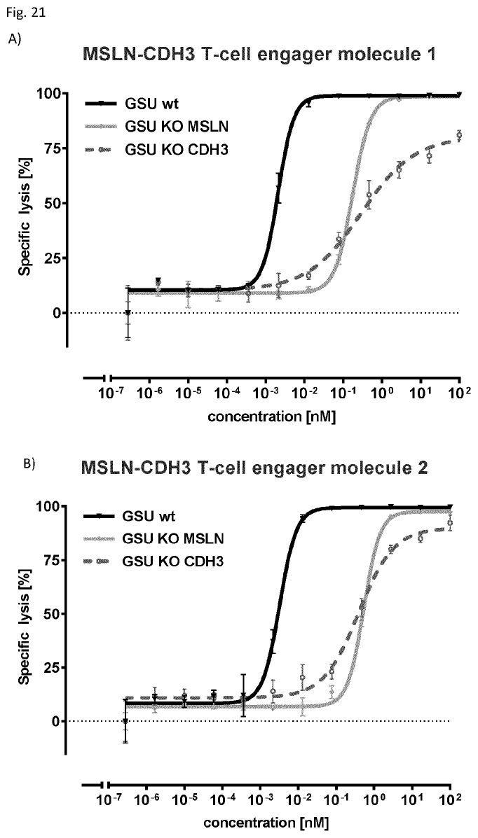

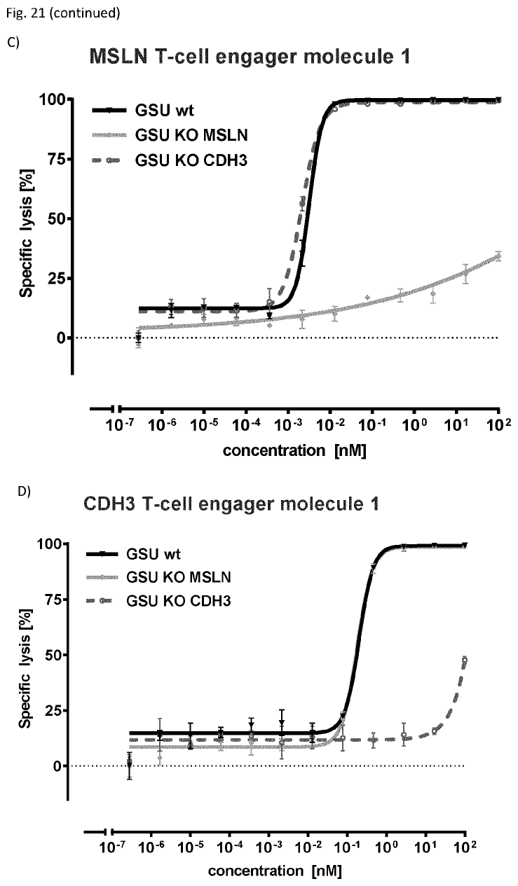

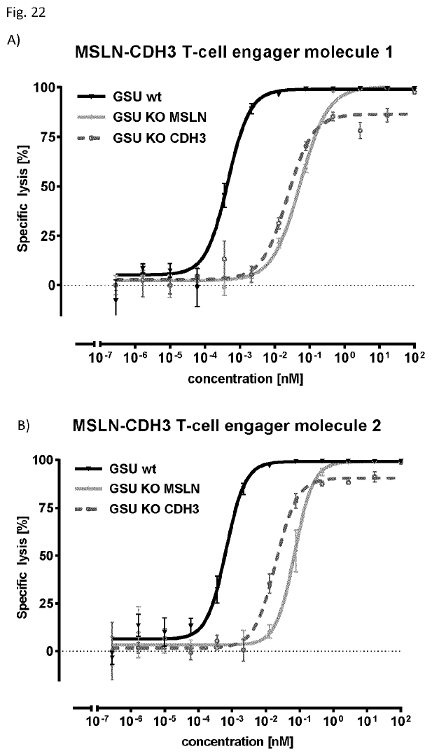

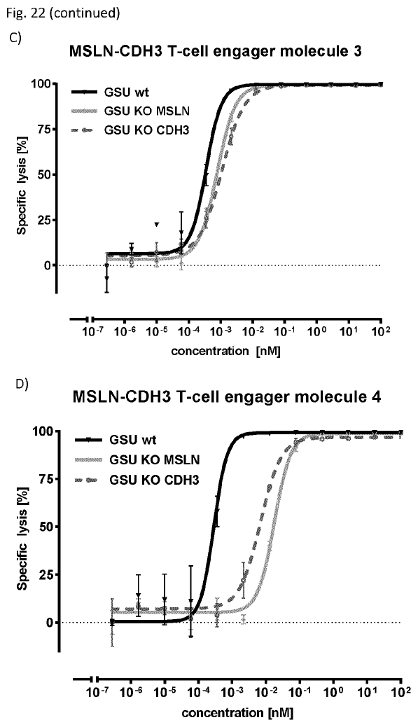

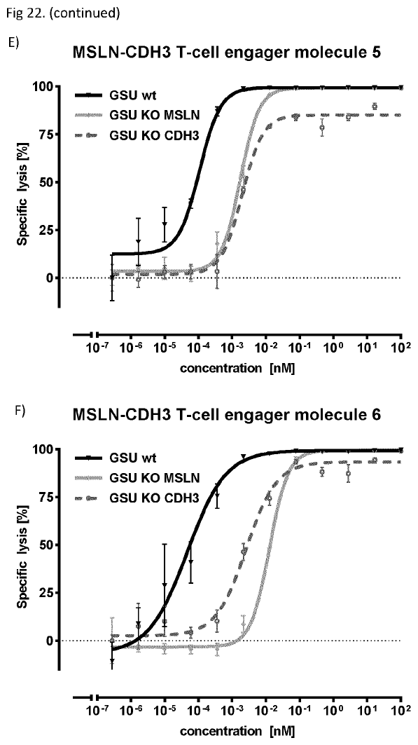

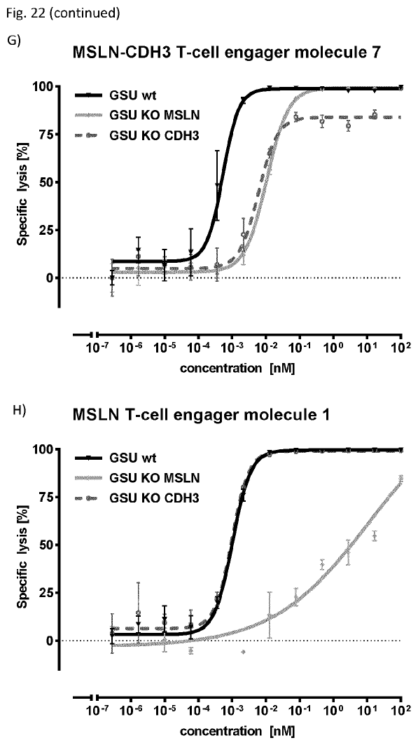

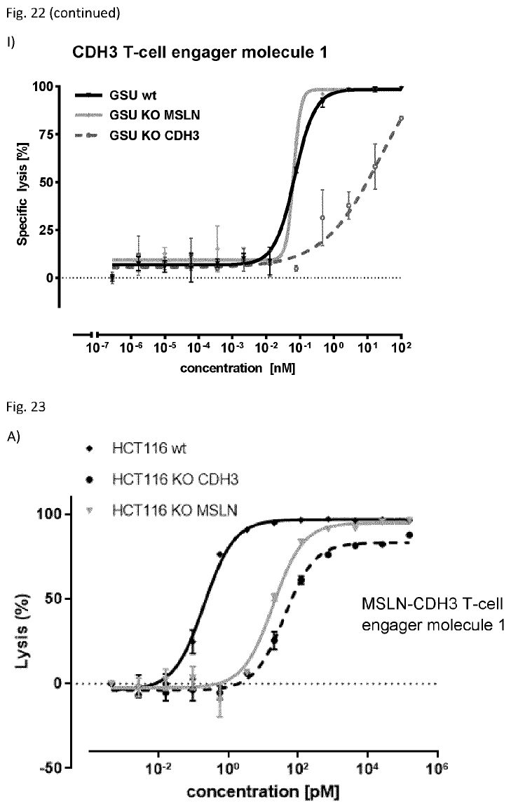

b.c.t .: below calculation threshold (see also FIG. 22)

TABLE 45
Activity reduction of CD3 binding domains used in MSLN-
CDH3 T-cell engager molecules compared to high affinity CD3
binding domain I2C with K_{D} of 1.2E−08M

| | Activity reduction of |
|---|---|
| | single CD3 binding domain |
| | in molecule compared to |
| | high affinity binding |
| | domain I2C with K_{D} 1.2E−08M |
| MSLN-CDH3 T-cell | Ca 100-fold |
| engager molecule 1 | |
| MSLN-CDH3 T-cell | Ca 98-fold |
| engager molecule 2 | |
| MSLN-CDH3 T-cell | I2C |
| engager molecule 3 | |
| MSLN-CDH3 T-cell | Ca 6-11 |
| engager molecule 4 | |
| MSLN-CDH3 T-cell | Ca 32-fold |
| engager molecule 5 | |
| MSLN-CDH3 T-cell | Ca 32- resp. 98-fold |
| engager molecule 6 | |
| MSLN-CDH3 T-cell | Ca 98- resp. 32-fold |
| engager molecule 7 | |

Results: MSLN-CDH3 T-cell engager molecules 1, 2, 4, 5, 6 and 7 demonstrated an EC50 selectivity gap between double positive GSU wt cells compared to respective GSU KO cells (GSU KO CDH3 and GSU KO MSLN) greater 10-fold, whereat MSLN-CDH3 T-cell engager molecule 1 and 2 showed the best selectivity gap. MSLN-CDH3 T-cell engager molecules 1, 2, 4 and 5 contain two identical CD3-binding domains that are between 11- to 100-fold less active than the reference CD3 binding domain I2C with an K_{D} of 1.2E-08M. The two CD3-binding domains in MSLN-CDH3 T-cell engager molecules 6 and 7 are not identical and still demonstrated an activity gap between GSU wt and respective GSU KO cells, with a low EC50 value on double positive cell. MSLN-CDH3 T-cell engager molecule 3 contains two high affinity CD3 binding domains I2C and only showed an increase in activity of maximum 3-fold on double positive vs. single positive cells. Mono targeting control T-cell engager molecules had comparable activity on single positive vs. double positive cells (selectivity gap of ˜1).

Legend

| | |
|---|---|
| MSLN-CDH3 T-cell | MS 15-B12 CC × I2C 44/100cc × scFc × |
| engager molecule 1 | CH3 15-E11 CC × I2C 44/100cc |
| MSLN-CDH3 T-cell | MS 15-B12 CC × I2L × scFc × |
| engager molecule 2 | CH3 15-E11 CC × I2L |
| MSLN-CDH3 T-cell | MS 15-B12 CC × I2C × scFc × |
| engager molecule 3 | CH3 15-E11 CC × I2C0 |
| MSLN-CDH3 T-cell | MS 15-B12 CC × 4F10.03 I2M × scFc × |
| engager molecule 4 | CH3 15-E11 CC × 4F10.03 I2M |
| MSLN-CDH3 T-cell | MS 15-B12 CC × I2M2 × scFc × CH3 |
| engager molecule 5 | 15-E11 CC × I2M2 |
| MSLN-CDH3 T-cell | MS 15-B12 CC × I2M2 × scFc × CH3 |
| engager molecule 6 | 15-E11 CC × I2L |
| MSLN-CDH3 T-cell | MS 15-B12 CC × I2L × scFc × CH3 15-E11 |
| engager molecule 7 | CC × I2M2 |
| MSLN T-cell | MSLN 5F11 × I2C -scFc |
| engager molecule 1 | |
| CDH3 T-cell | CH3 G8A 6-B12 × I2C0-scFc |
| engager molecule 1 | |

### Example 21: Selectivity Gap of Multitargeting Bispecific T-Cell Engagers Targeting Different CDH3 and MSLN Epitope Clusters

Luciferase-Based Assay with Unstimulated Human PBMC

### Isolation of Effector Cells

Human peripheral blood mononuclear cells (PBMC) were prepared by Ficoll density gradient centrifugation from enriched lymphocyte preparations (buffy coats), a side product of blood banks collecting blood for transfusions. Buffy coats were supplied by a local blood bank and PBMC were prepared on the day after blood collection. After Ficoll density centrifugation and extensive washes with Dulbecco's PBS (Gibco), remaining erythrocytes were removed from PBMC via incubation with erythrocyte lysis buffer (155 mM NH₄Cl, 10 mM KHCO₃, 100 μM EDTA). Remaining lymphocytes mainly encompass B and T lymphocytes, NK cells and monocytes. PBMC were kept in culture at 37° C./5% CO₂ in RPMI medium (Gibco) with 10% FCS (Gibco).

Depletion of CD14⁺ and CD56⁺ Cells

For depletion of CD14+ cells, human CD14 MicroBeads (Milteny Biotec, MACS, #130-050-201) were used, for depletion of NK cells human CD56 MicroBeads (MACS, #130-050-401). PBMC were counted and centrifuged for 10 min at room temperature with 300×g. The supernatant was discarded and the cell pellet resuspended in MACS isolation buffer (60 μL/10⁷ cells). CD14 MicroBeads and CD56 MicroBeads (20 μL/10⁷ cells) were added and incubated for 15 min at 4-8° C. The cells were washed with AutoMACS rinsing buffer (Milteny #130-091-222) (1-2 mL/10⁷ cells). After centrifugation (see above), supernatant was discarded and cells resuspended in MACS isolation buffer (500 μL/10⁸ cells). CD14/CD56 negative cells were then isolated using LS Columns (Milteny Biotec, #130-042-401). PBMC w/o CD14+/CD56+ cells were adjusted to 1.2×10⁶ cells/mL and cultured in RPMI complete medium i.e. RPMI1640 (Biochrom AG, #FG1215) supplemented with 10% FBS (Bio West. #S1810), 1× non-essential amino acids (Biochrom AG, #K0293), 10 mM Hepes buffer (Biochrom AG, #L1613), 1 mM sodium pyruvate (Biochrom AG, #L0473) and 100 U/mL penicillin/streptomycin (Biochrom AG, #A2213) at 37° ° C. in an incubator until needed.

### Target Cell Preparation

Cells were harvested, spinned down and adjusted to 1.2×10⁵ cells/mL in complete RPMI medium. The vitality of cells was determined using Nucleocounter NC-250 (Chemometec) and Solution18 Dye containing Acridine Orange and DAPI (Chemometec).

### Luciferase Based Analysis

This assay was designed to quantify the lysis of target cells in the presence of serial dilutions of multi-specific antibody constructs. Equal volumes of Luciferase-positive target cells and effector cells (i.e., PBMC w/o CD14⁺; CD56⁺ cells) were mixed, resulting in an E:T cell ratio of 10:1. 42 μL of this suspension were transferred to each well of a 384-well plate. 8 μL of serial dilutions of the corresponding multi-specific antibody constructs and a negative control antibody constructs (a CD3-based antibody construct recognizing an irrelevant target antigen) or RPMI complete medium as an additional negative control were added. The multi-specific antibody-mediated cytotoxic reaction proceeded for 48 hours in a 5% CO₂ humidified incubator. Then 25 μL substrate (Steady-Glo® Reagent, Promega) were transferred to the 384-well plate. Only living, Luciferase-positive cells react to the substrate and thus create a luminescence signal. Samples were measured with a SPARK microplate reader (TECAN) and analyzed by Spark Control Magellan software (TECAN).

Percentage of cytotoxicity was calculated as follows:

$$Cytoxicity [ \% ] = ( 1 - \frac{R L {U}_{Sample}}{R L {U}_{Negative ‐ Contro1}} ) \times 100$$

RLU=relative light units

Negative-Control=cells without multi-specific antibody construct

Using GraphPad Prism 7.04 software (Graph Pad Software, San Diego), the percentage of cytotoxicity was plotted against the corresponding multi-specific antibody construct concentrations. Dose response curves were analyzed with the four parametric logistic regression models for evaluation of sigmoid dose response curves with fixed hill slope and EC50 values were calculated.

Following target cell lines were used for the Luciferase-based cytotoxicity assay:
HCT 116-LUC wt (CDH3+ and MSLN+)
HCT 116-LUC KO CDH3 (CDH3− and MSLN+)
HCT 116-LUC KO MSLN (CDH3+ and MSLN−)
CHO human CDH3+ and MSLN+
CHO human CDH3+
CHO human MSLN+

TABLE 46
EC50 values of MSLN-CDH3 T-cell engagers, targeting different
CDH3 epitope clusters on respective cell lines.

| | EC50 [pM] |
|---|---|
| MSLN-CDH3 T-cell engager | 0.07 |
| molecule 1 on HCT 116 wt | |
| MSLN-CDH3 T-cell engager | 51.8 |
| molecule 1 on HCT 116 KO CDH3 | |
| MSLN-CDH3 T-cell engager | 8.7 |
| molecule 1 on HCT 116 KO MSLN | |
| MSLN-CDH3 T-cell engager | 0.201 |
| molecule 2 on HCT 116 wt | |
| MSLN-CDH3 T-cell engager | 19.3 |
| molecule 2 on HCT 116 KO CDH3 | |
| MSLN-CDH3 T-cell engager | 40.9 |
| molecule 2 on HCT 116 KO MSLN | |
| MSLN-CDH3 T-cell engager | 1.557 |
| molecule 3 on HCT 116 wt | |
| MSLN-CDH3 T-cell engager | 85.41 |
| molecule 3 on HCT 116 KO CDH3 | |
| MSLN-CDH3 T-cell engager | 413.3 |
| molecule 3 on HCT 116 KO MSLN | |
| MSLN-CDH3 T-cell engager | 5.53 |
| molecule 4 on HCT 116 wt | |
| MSLN-CDH3 T-cell engager | 267.87 |
| molecule 4 on HCT 116 KO CDH3 | |
| MSLN-CDH3 T-cell engager | 495.43 |
| molecule 4 on HCT 116 KO MSLN | |
| MSLN-CDH3 T-cell engager | 1.27 |
| molecule 5 on HCT 116 wt | |
| MSLN-CDH3 T-cell engager | 58.54 |
| molecule 5 on HCT 116 KO CDH3 | |
| MSLN-CDH3 T-cell engager | 71.0 |
| molecule 5 on HCT 116 KO MSLN | |

### Legend for CDH3 Epitope Cluster Analysis:

MSLN-CDH3 T-cell engager molecule 1: CH3 15-E11 CC×I2L×G4×scFc xG4×MS 15-B12 CC×I2L_GQ

MSLN-CDH3 T-cell engager molecule 2: MS 15-B12 CC×I2L×(G4S)3×scFc×(G4S)3×CH3 24-D7 CC×I2L

MSLN-CDH3 T-cell engager molecule 3: MS 15-B12 CC×I2L×G4×scFc×G4×CH3 22-A12 CC×I2L

MSLN-CDH3 T-cell engager molecule 4: MS 15-B12 CC×I2L×G4×scFc×G4×CH3 005-D5 CC×I2L

MSLN-CDH3 T-cell engager molecule 5: MS 15-B12 CC×I2L×(G4S)3×scFc×(G4S)3×CH3 26-E5 CC×I2L

Positive control molecule 1: MS 5-F11×I2C scFc6

Positive control molecule 2: CH3 G8A 6-B12×I2C0-scFc

Negative control molecule 1: EGFRVIII×I2C0×scFc

### Results:

TABLE 47
EC50 values in pM and gaps of naïve
HCT 116 cells versus knock-out HCT 116 cells

| | | | EC50 CHO | | |
|---|---|---|---|---|---|
| | EC50 CHO hu | | hu CDH3 (+) | | EC50 CHO |
| | MSLN (+) | | & MSLN (+) | | hu CDH3 (+) |
| | [pM] | fold gap | [pM] | fold gap | [pM] |
| MSLN-CDH3 T-cell | 8.7 | 122 | 0.071 | 727 | 51.8 |
| engager molecule 1 | | | | | |
| MSLN-CDH3 T-cell | 19.3 | 96 | 0.201 | 203 | 40.9 |
| engager molecule 2 | | | | | |
| MSLN-CDH3 T-cell | 413.3 | 265 | 1.557 | 55 | 85.4 |
| engager molecule 3 | | | | | |
| MSLN-CDH3 T-cell | 445.4 | 90 | 5.53 | 48 | 267.9 |
| engager molecule 4 | | | | | |
| MSLN-CDH3 T-cell | 71 | 56 | 1.272 | 46 | 58.5 |
| engager molecule 5 | | | | | |

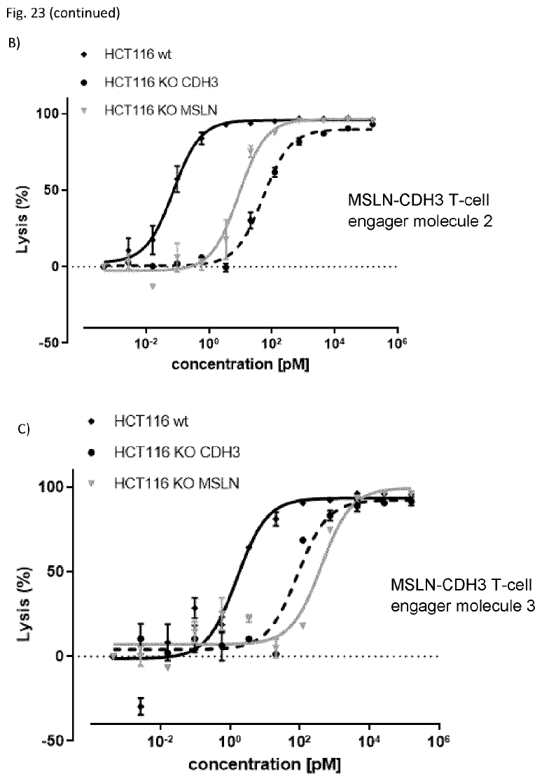

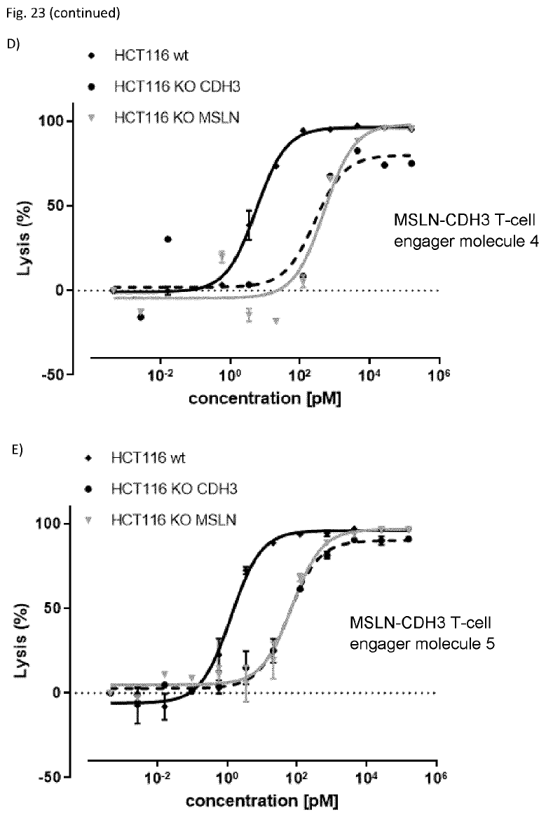

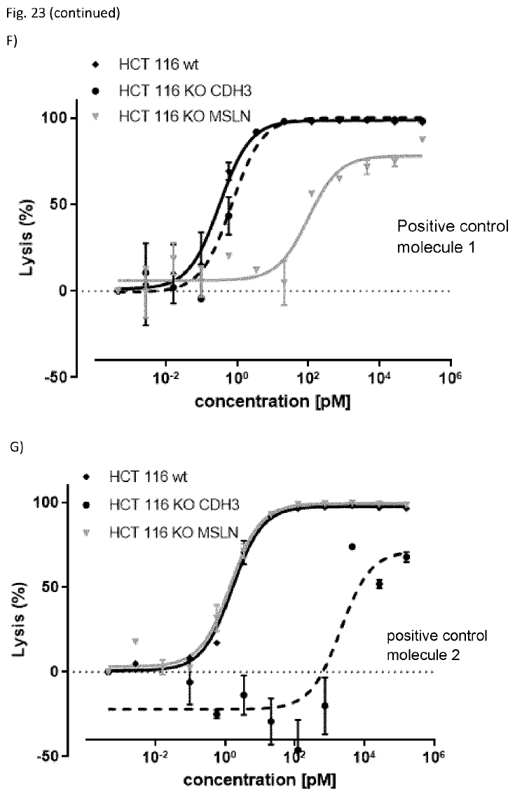

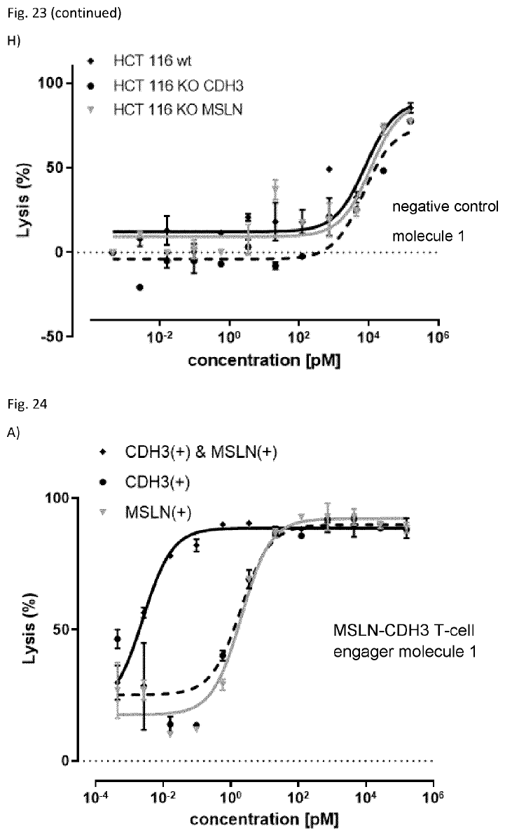

The tested MSLN-CDH3 T-cell engager molecules showed all increased activity (lower EC50 values) on MSLN and CDH3 double positive HCT 116 wt cells compared to respective HCT 116 k.o cells (HCT 116 CDH3 k.o and HCT 116 MSLN k.o.). The MSLN-CDH3 T-cell engager molecules 1 and 2 showed EC50 selectivity gaps greater ˜100-fold (on both sites) on double positive target cells versus single positive target cells and are, thus, preferred (FIG. 23) and (Table 47). The tested MSLN-CDH3 T-cell engager molecules 3, 4 & 5 showed EC50 selectivity gaps lower 100-fold on double positive target cells versus single positive target cells (FIG. 23) and (Table 47). The tested MSLN-CDH3 T-cell engager molecules 1&2 contain CDH3 binder of the epitope D4B. The tested MSLN-CDH3 T-cell engager molecules 3 contains a CDH3 binder of the epitope D1B. The tested MSLN-CDH3 T-cell engager molecule 4 contains a CDH3 binder for the epitope D2C. The tested MSLN-CDH3 T-cell engager molecules 5 contains a CDH3 binder of for epitope D3A.

### Legend for MSLN Epitope Cluster Analysis:

MSLN-CDH3 T-cell engager molecule 1: MS 01-G11 CC×6H10.09×(G4S)3×scFc×(G4S)3×CH3 005-D5 CC×6H10.09

MSLN-CDH3 T-cell engager molecule 2: MS R4L CC×I2C CC (44/100)×(G4S)3×scFc×(G4S)3×CH3 R164L CC×I2C CC (44/100)

Positive control molecule 1: MS 5-F11×I2C scFc6

Positive control molecule 2: CH3 G8A 6-B12×I2C0-scFc

Negative control molecule 1: EGFRVIII×12C0×scFc ID:

### Results:

TABLE 48
EC50 values in pM and gaps of double positive human target
CHO cells versus single positive human target cells

| | | | EC50 CHO | | |
|---|---|---|---|---|---|
| | EC50 CHO hu | | hu CDH3 (+) | | EC50 CHO |
| | MSLN (+) | | & MSLN (+) | | hu CDH3 (+) |
| | [pM] | fold gap | [pM] | fold gap | [pM] |
| MSLN-CDH3 T-cell | 2.05 | 822 | 0.002 | 779 | 1.9 |
| engager molecule 1 | | | | | |
| MSLN-CDH3 T-cell | 2.53 | 70 | 0.036 | 139 | 5.8 |
| engager molecule 2 | | | | | |

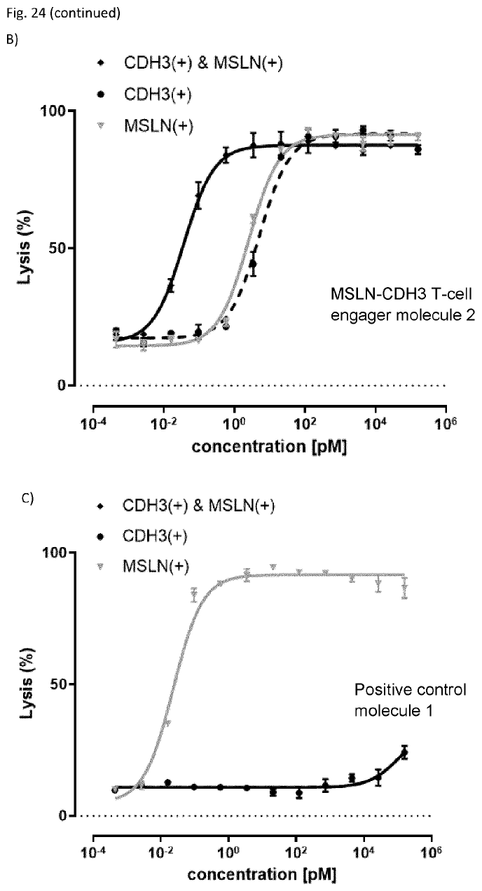

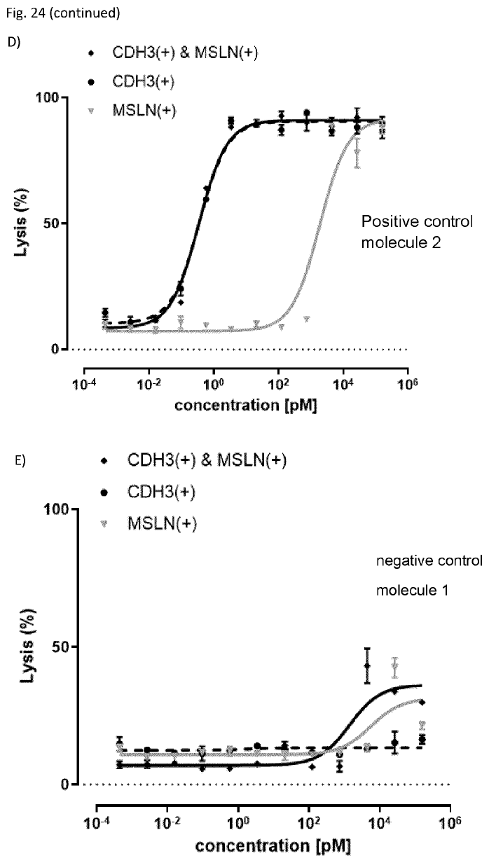

The tested MSLN-CDH3 T-cell engager molecule 1 and 2 showed increased activity (lower EC50 values) on human MSLN and CDH3 double positive CHO cells compared to respective human target single positive CHO cells. The MSLN-CDH3 T-cell engager molecule 1 showed EC50 selectivity gaps greater 100-fold (on both sites) on double positive target cells versus single positive target cells. (FIG. 24) and (Table 48). The tested MSLN-CDH3 T-cell engager molecule 2 showed EC50 selectivity gaps lower 100-fold (on one site) on double positive target cells versus single positive target cells (FIG. 24) and (Table 48). The tested MSLN-CDH3 T-cell engager molecules 1 contains a MSLN binder for the epitope E1. The tested MSLN-CDH3 T-cell engager molecule 2 contains a MSLN binder for the epitope E2/E3. While molecule 2 shows good selectivity, molecule 1 is preferred.

### Example 22: Epitope Clustering of Ta Cell Engager with Chimeric Human/Mouse CDH3 Proteins

### Construct Generation

The human CDH3 protein extracellular region was divided into five parts: (1) domain 1, designated D1, (2) domain 2, designated D2, (3) domain 3, designated D3, (4) domain 4, designated D4 and (5) domain 5, designated D5. The epitope regions D1, D2, D3, D4 and D5 were further divided into three subparts, designated DIA, D1B, DIC, D2A, D2B, D2C, D3A, D3B, D3C, D4A, D4B, D4C, D5A, DSB and D5C.

TABLE 50
D2B, D2C, D3A and D4B have the following
amino acid sequence and positions (aa)
of the human CDH3 protein:

| Human | | | Amino acid |
|---|---|---|---|
| CDH3 | | Amino | sequence of |
| epitope | | acid | CDH3 epitope |
| cluster | species | position | cluster |
| D2B | human | aa 253-290 | VAYSIHSQEPKDPHD |
| | | | LMFTIHRSTGTISVI |
| | | | SSGLDREK |
| D2C | human | aa 291-327 | VPEYTLTIQATDMDG |
| | | | DGSTTTAVAVVEILD |
| | | | ANDNAPM |
| D3A | human | aa 328-363 | FDPQKYEAHVPENAV |
| | | | GHEVQRLTVTDLDAP |
| | | | NSPAWR |
| D4B | human | aa 476-511 | YRILRDPAGWLAMDP |
| | | | DSGQVTAVGTLDRED |
| | | | EQFVRN |

The human/mouse chimeric proteins were generated by replacing domains D1, D2, D3, D4, D5 or the respective subparts of the human CDH3 protein with the corresponding regions from mouse CDH3 protein.

The extracellular domain of mouse CDH3 and all chimeric human/mouse CDH3 constructs are fused to the transmembrane and cytoplasmic domain of EpCAM what is of no significance for the assay described here and is designated xEpC hereafter. The protein sequence of each of the constructs described above is depicted in FIG. 25. Deoxyribonucleic acid (DNA) sequences encoding either full-length human CDH3, mouse CDH3×EpC protein or human/mouse chimeric CDH3×EpC proteins were each cloned into a pEFdhfr vector and stably transfected into CHO dihydrofolate reductase-negative (DHFR−) cells. The aforementioned method can be applied with respect to any antigen-binding molecule binding CDH3 of the invention.

### Transfection

CHO DHFR− cells were transfected according to standard protocols with DNA encoding either the human CDH3 protein, the mouse CDH3×EpC protein or chimeric human/mouse CDH3×EpC proteins. Cells were grown in RPMI Medium with supplements for 24 hours. Selection of adherent-growing cells expressing human CDH3, mouse CDH3×EpC or chimeric human/mouse CDH3×EpC protein by nucleoside deprivation was done after 24 hours and cells were cultured in HyClone Medium with Pen/Strep at 37° in a humidified incubator.

### Flow Cytometry

To verify expression of the human CDH3 protein or chimeric human/mouse CDH3×EpC proteins on stably transfected CHO, cells were incubated with 5 μg/mL of an anti-human CDH3 antibody (R&D Systems, clone 104805) and 1:100 dilution of PE-labeled anti mouse Fcy secondary antibody (Jackson 115-116-071). To verify expression of the mouse CDH3×EpC protein on stably transfected CHO, cells were incubated with a periplasmic extract of the mouse cross reactive anti-human CDH3 scFv G7 (diluted 1:6 with PBS) and 1:50 dilution of a PE-labeled anti FLAG antibody (clone L5; BioLegend 637310). To evaluate binding of T cell engager SEQ ID NO: 434 to proteins expressed on the transfected cells, cells were incubated with 5 μg/mL of the T-cell engager SEQ ID NO: 434. Binding of the T cell engager SEQ ID NO: 434 was detected using a 1:50 dilution of a PE-labeled anti-human Fcy antibody. All antibodies were diluted in PBS with Calcium (Gibco 14040-117) and 2% FBS and all incubations were performed at 4° C. for 30 minutes. Washes were done using PBS with Calcium (Gibco 14040-117) and 2% FBS and the final suspension buffer prior to FACS analysis was also PBS with Calcium (Gibco 14040-117) and 2% FBS. Antibody binding was detected using a BD FACSCanto® II flow cytometer. Changes in mean fluorescence were analyzed with BD FACSDiva®, v8.1, ForeCyt® and FlowJo®.

### Analysis

Loss of binding to the various human/mouse chimeric CDH3 proteins was reflected as a decrease in signal detected by flow cytometry.

### Results

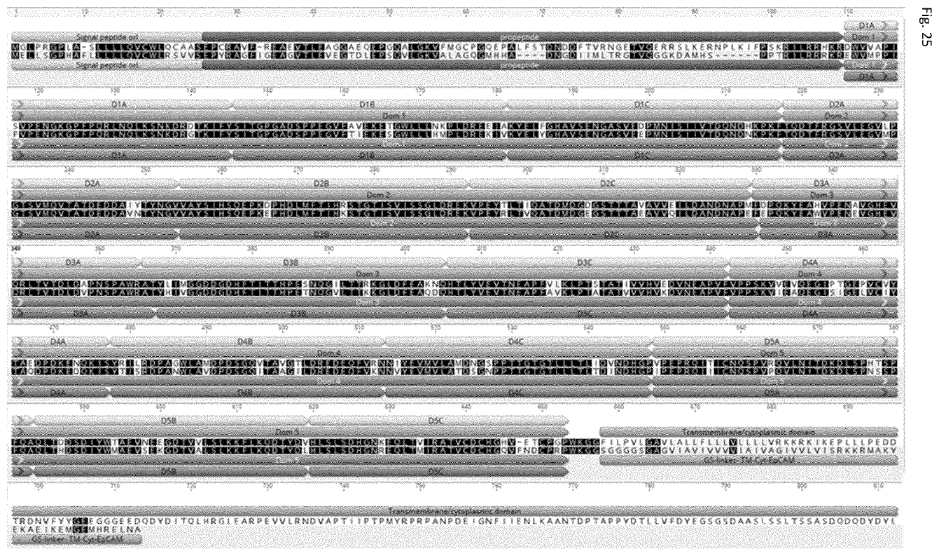

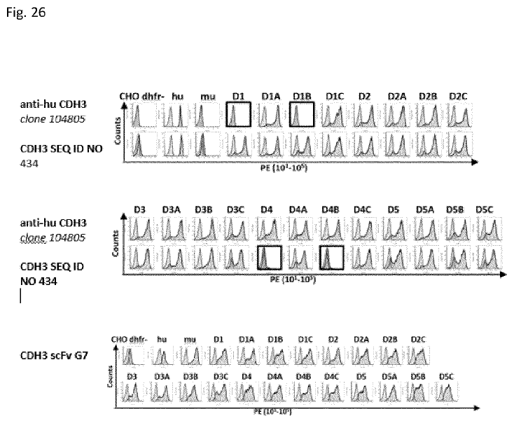

FIG. 25 depicts alignments of protein sequences of human CDH3 and mouse CDH3×EpC with colored epitope sections. As generically applicable with respect to the present invention, the extracellular domain 1 of CDH3 protein was designated D1, the following domains 2, 3, 4 and 5 were designated D2, D3, D4 and D5. For more refined epitope clustering, the extracellular domains D1, D2, D3, D4 and D5 were further divided into the subparts A, B, and C. For the epitope clustering, chimeric human/mouse CDH3 proteins were generated in which regions of human CDH3 protein were replaced with the corresponding regions from mouse CDH3 protein. Because T cell engager SEQ ID NO: 434 and anti-human CDH3 antibody do not bind the mouse CDH3 protein (FIG. 26), the binding epitope region can be identified by systematically replacing sections of the human protein with the mouse protein (human/mouse CDH3 chimeras) and determining which chimera is no longer recognized by the T cell engager SEQ ID NO: 434. Human CDH3, mouse CDH3×EpC and chimeric human/mouse CDH3×EpC proteins were stably expressed in CHO cells and binding of T Cell engager SEQ ID NO: 434, anti-human CDH3 antibody and mouse cross reactive anti human CDH3 scFv G7 to surface-expressed proteins was assessed by flow cytometry (FIG. 26).

T cell engager SEQ ID NO: 434 bound to cells expressing full-length human CDH3 protein, indicating it recognized the human extracellular domain. T cell engager SEQ ID NO: 434 did not bind to cells expressing mouse CDH3 protein, indicating it did not recognize the mouse extracellular domain. When binding to the domain-swapped proteins was evaluated, T cell engager SEQ ID NO: 434 showed binding to all human/mouse chimeric CDH3 proteins except D4 and D4B. If the human D4 or D4B domain was replaced with the mouse D4 or D4B domain respectively, SEQ ID NO: 434 did not recognize the chimeric protein. Binding of SEQ ID NO: 434 was not affected by exchange of D1, D2, D3, D5 or their respective subparts A, B or C. In conclusion, T cell engager SEQ ID NO: 434 shows a loss of binding to the epitope cluster D4B.

### Example 23

Epitope clustering of T cell engager SEQ ID NO: 434 and SEQ ID NO: 251 with chimeric human/mouse MSLN proteins

### Construct Generation

The mature human MSLN protein extracellular region was divided into six parts (designated hereafter epitope section): (1) epitope section 1 designated E1, (2) epitope section 2, designated E2, (3) epitope section 3, designated E3, (4) epitope section 4, designated E4, (5) epitope section 5, designated E5 and (6) epitope section 6, designated E6.

TABLE 51
E1, E2, E3, E4 and E5 have the following
amino acid sequence and positions (aa)
of the human MSLN protein:

| Human | | | amino acid |
|---|---|---|---|
| MSLN | | amino | sequence of |
| epitope | | acid | MSLN epitope |
| cluster | species | position | cluster |
| E1 | human | aa 296- | EVEKTACPSGKKAR |
| | | 346 | EIDESLIFYKKWEL |
| | | | EACVDAALLATQMD |
| | | | RVNAIPFTY |
| E2 | human | aa 347- | EQLDVLKHKLDELY |
| | | 384 | PQGYPESVIQHLGY |
| | | | LFLKMSPEDI |
| E3 | human | aa 385- | RKWNVTSLETLKAL |
| | | 453 | LEVNKGHEMSPQVA |
| | | | TLIDRFVKGRGQLD |
| | | | KDTLDTLTAFYPGY |
| | | | LCSLSPEELSSVP |
| E4 | human | aa 454- | PSSIWAVRPQDLDT |
| | | 501 | CDPRQLDVLYPKAR |
| | | | LAFQNMNGSEYFVK |
| | | | IQSFLG |
| E5 | human | aa 502- | GAPTEDLKALSQQN |
| | | 545 | VSMDLATFMKLRTD |
| | | | AVLPLTVAEVQKLL |
| | | | GP |

The human/mouse chimeric proteins were generated by replacing epitope sections E1, E2, E3, E4, E5 or E6 of the human MSLN protein with the corresponding region from mouse MSLN protein. The protein sequence of each of the constructs described above is depicted in FIG. 1. Deoxyribonucleic acid (DNA) sequences encoding either full-length human, mouse or human/mouse chimeric MSLN proteins were each cloned into a pEFdhfr vector and stably transfected into CHO dihydrofolate reductase-negative (DHFR−) cells.

### Transfection

CHO DHFR− cells were transfected according to standard protocols with DNA encoding either the full-length human MSLN protein, the full-length mouse MSLN protein or chimeric human/mouse MSLN proteins. Cells were grown in RPMI Medium with supplements for 24 hours. Selection of adherent-growing cells expressing human MSLN, mouse MSLN or chimeric human/mouse MSLN proteins by nucleoside deprivation was done after 24 hours and cells were cultured in HyClone Medium with Pen/Strep at 37° in a humidified incubator.

### Flow Cytometry

To verify expression of the human MSLN protein or the chimeric human/mouse MSLN proteins on stably transfected CHO, cells were incubated with 5 μg/mL of an anti-human MSLN antibody (Thermo Fischer MA1-26527, clone 1) and 1:100 dilution of PE-labeled anti mouse Fcy secondary antibody (Jackson 115-116-071). To verify expression of the mouse MSLN protein on stably transfected CHO, cells were incubated with 5 μg/mL of the mouse cross reactive anti-human MSLN BITE R4T and a 1:50 dilution of a PE-labeled anti-human Fcy antibody (Jackson 109-116-098). To evaluate binding of T cell engager SEQ ID NO: 434 and SEQ ID NO: 251 to proteins expressed on the transfected cells, cells were incubated with 5 μg/mL of the T-cell engager SEQ ID NO: 434 or SEQ ID NO: 251. Binding of the T cell engager SEQ ID NO: 434 and SEQ ID NO: 251 was detected using a 1:50 dilution of a PE-labeled anti-human Fcy antibody. All antibodies were diluted in PBS with 2% FBS and all incubations were performed at 4° C. for 30 minutes. Washes were done using PBS with 2% FBS and the final suspension buffer prior to FACS analysis was also PBS with 2% FBS.

Antibody binding was detected using a BD FACSCanto® II flow cytometer. Changes in mean fluorescence were analyzed with BD FACSDiva®, v8.1, ForeCyt® and FlowJo®.

### Analysis

Loss of the binding to the various human/mouse chimeric MSLN proteins was reflected as a decrease in signal detected by flow cytometry.

### Results

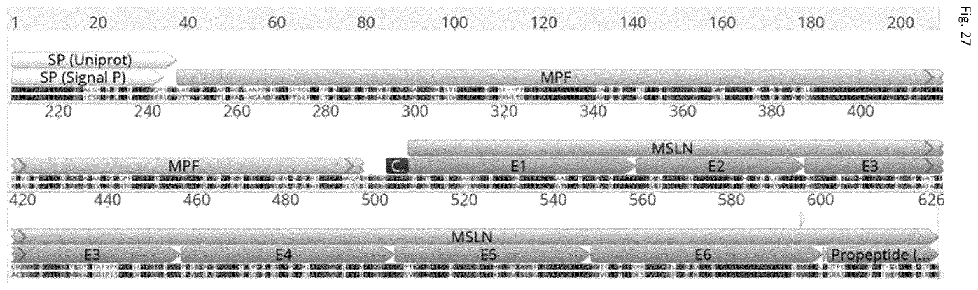

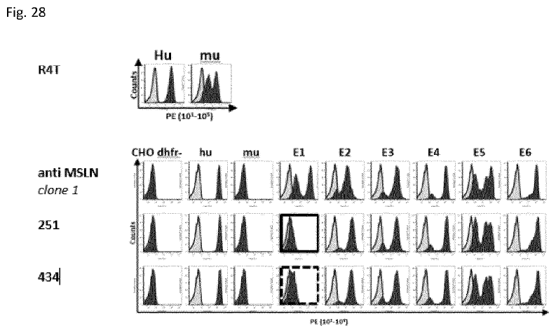

FIG. 27 depicts alignments of protein sequences of human MSLN and mouse MSLN with colored epitope sections. The extracellular domain of the MSLN protein was divided into six parts designated epitope sections. As generally applicable in the context of the present invention, epitope section 1 of the MSLN protein was designated E1, epitope sections 2, 3, 4, 5 and 6 were designated E2, E3, E4, E5 and E6 respectively. For the epitope clustering, chimeric human/mouse MSLN proteins were generated in which regions of human MSLN protein were replaced with the corresponding regions from mouse MSLN protein. Because T cell engager SEQ ID NO: 434 and SEQ ID NO: 251 do not bind the mouse MSLN protein (FIG. 28), the binding epitope region can be identified by systematically replacing sections of the human protein with the mouse protein (human/mouse MSLN chimeras) and determining which chimera is no longer recognized by the T cell engager SEQ ID NO: 434 and SEQ ID NO: 251. Human MSLN, mouse MSLN and chimeric human/mouse MSLN proteins were stably expressed in CHO cells and binding of T Cell engager SEQ ID NO: 434 and SEQ ID NO: 251 and anti-human MSLN antibody to surface-expressed proteins was assessed by flow cytometry (FIG. 28). T cell engager SEQ ID NO: 434 and SEQ ID NO: 251 bound to cells expressing full length human MSLN protein, indicating it recognized the human extracellular domain. T cell engager SEQ ID NO: 434 and SEQ ID NO: 251 did not bind to cells expressing mouse MSLN protein, indicating it did not recognize the mouse extracellular domain. When binding to the domain-swapped proteins was evaluated, T cell engager SEQ ID NO: 434 and SEQ ID NO: 251 showed binding to human/mouse chimeric MSLN proteins E2, E3, E4, E5 and E6. If the human E1 epitope section of MSLN was replaced with the respective mouse E1 epitope section, SEQ ID NO: 434 and SEQ ID NO: 251 did not recognize the chimeric protein. Binding of SEQ ID NO: 434 and SEQ ID NO: 251 was not affected by exchange of the human sequence of E2, E3, E4, E5 or E6 to the respective mouse sequence.

In CONCLUSION, representative T-cell engager of the invention SEQ ID NO: 434 and SEQ ID NO: 251 shows a loss of binding to the MSLN epitope cluster E1.

TABLE 53
Sequence Table: Linkers, which may be indicated in the
description as “G4”, “(G4S)n”, “(G4Q)n” or the like are
not necessarily indicated in the table with such linked
binding domain in order to maintain readability. The
absence of such linker indication does not mean that
the molecule in the table differs from the correspond-
ing molecule in the description under a denomination
which comprises the linker information. “CC” indicates
disulfide bonds within a binding domain, “I2L”, “I2C”,
“I2M” and “2M2” indicate CD3 binding domains. Target
binding domains may be abbreviated such as “CH3” for
“CDH3”, “CL1” for “CLL1”, “FL” for “FLT3” and “MS” for
“MSLN”.

| SEQ | | | | |
|---|---|---|---|---|
| ID | | | | |
| NO: | Designation | Source | | Sequence |
| 1. | (G4Q)3 - Linker | artificial | Aa | GGGGQGGGGQGGGGQ |
| 2. | (G4S)10 - Linker | artificial | aa | GGGGSGGGGSGGGGSGGGGSGGGGSGGGGSG |
| | | | | GGGSGGGGSGGGGSGGGGS |
| 3. | (G4S)3 - Linker | artificial | aa | GGGGSGGGGSGGGGS |
| 4. | G(EAAAK)10 - | artificial | aa | GEAAAKEAAAKEAAAKEAAAKEAAAKEAAA |
| | Linker | | | KEAAAKEAAAKEAAAKEAAAK |
| 5. | G4 - Linker | artificial | aa | GGGG |
| 6. | G4Q - Linker | artificial | aa | GGGGQ |
| 7. | G4S - Linker, | artificial | aa | GGGGS |
| | spacer control | | | |
| 8. | S(G4S)10 - | artificial | aa | SGGGGSGGGGSGGGGSGGGGSGGGGSGGGGS |
| | Linker | | | GGGGSGGGGSGGGGSGGGGS |
| 9. | S(G4S)3 - Linker | artificial | aa | SGGGGSGGGGSGGGGS |
| 10. | SG(EAAAK)10 - | artificial | aa | SGEAAAKEAAAKEAAAKEAAAKEAAAKEAA |
| | Linker | | | AKEAAAKEAAAKEAAAKEAAAK |
| 11. | SG4Q - Linker | artificial | aa | SGGGGQ |
| 12. | SG4S - Linker | artificial | aa | SGGGGS |
| 13. | (EAAAK)10 - | artificial | aa | EAAAKEAAAKEAAAKEAAAKEAAAKEAAAK |
| | Spacer | | | EAAAKEAAAKEAAAKEAAAK |
| 14. | (G4S)10 - Spacer | artificial | aa | GGGGSGGGGSGGGGSGGGGSGGGGSGGGGSG |
| | control | | | GGGSGGGGSGGGGSGGGGS |
| 15. | Human Serum | artificial | Aa | DAHKSEVAHRFKDLGEENFKALVLIAFAQYLQ |
| | Albumin (HSA) - | | | QCPFEDHVKLVNEVTEFAKTCVADESAENCDK |
| | Spacer | | | SLHTLFGDKLCTVATLRETYGEMADCCAKQEP |
| | | | | ERNECFLQHKDDNPNLPRLVRPEVDVMCTAFH |
| | | | | DNEETFLKKYLYEIARRHPYFYAPELLFFAKRY |
| | | | | KAAFTECCQAADKAACLLPKLDELRDEGKASS |
| | | | | AKQRLKCASLQKFGERAFKAWAVARLSQRFP |
| | | | | KAEFAEVSKLVTDLTKVHTECCHGDLLECADD |
| | | | | RADLAKYICENQDSISSKLKECCEKPLLEKSHCI |
| | | | | AEVENDEMPADLPSLAADFVESKDVCKNYAE |
| | | | | AKDVFLGMFLYEYARRHPDYSVVLLLRLAKT |
| | | | | YETTLEKCCAAADPHECYAKVFDEFKPLVEEP |
| | | | | QNLIKQNCELFEQLGEYKFQNALLVRYTKKVP |
| | | | | QVSTPTLVEVSRNLGKVGSKCCKHPEAKRMPC |
| | | | | AEDYLSVVLNQLCVLHEKTPVSDRVTKCCTES |
| | | | | LVNRRPCFSALEVDETYVPKEFNAETFTFHADI |
| | | | | CTLSEKERQIKKQTALVELVKHKPKATKEQLK |
| | | | | AVMDDFAAFVEKCCKADDKETCFAEEGKKLV |
| | | | | AASQAALGL |
| 16. | PD1 (ECD 25- | artificial | Aa | LDSPDRPWNPPTFSPALLVVTEGDNATFTCSFS |
| | 167) - Spacer | | | NTSESFVLNWYRMSPSNQTDKLAAFPEDRSQP |
| | | | | GQDCRFRVTQLPNGRDFHMSVVRARRNDSGT |
| | | | | YLCGAISLAPKAQIKESLRAELRVTERRAEVPT |
| | | | | AHPSPSPRPAGQFQ |
| 17. | Fc monomer-1 - | artificial | Aa | DKTHTCPPCPAPELLGGPSVFLFPPKPKDTLMIS |
| | c/+g | | | RTPEVTCVVVDVSHEDPEVKFNWYVDGVEVH |
| | | | | NAKTKPCEEQYGSTYRCVSVLTVLHQDWLNG |
| | | | | KEYKCKVSNKALPAPIEKTISKAKGQPREPQVY |
| | | | | TLPPSREEMTKNQVSLTCLVKGFYPSDIAVEWE |
| | | | | SNGQPENNYKTTPPVLDSDGSFFLYSKLTVDKS |
| | | | | RWQQGNVFSCSVMHEALHNHYTQKSLSLSPG |
| | | | | K |
| 18. | Fc monomer-2 - | artificial | Aa | DKTHTCPPCPAPELLGGPSVFLFPPKPKDTLMIS |
| | c/+g/delGK | | | RTPEVTCVVVDVSHEDPEVKFNWYVDGVEVH |
| | | | | NAKTKPCEEQYGSTYRCVSVLTVLHQDWLNG |
| | | | | KEYKCKVSNKALPAPIEKTISKAKGQPREPQVY |
| | | | | TLPPSREEMTKNQVSLTCLVKGFYPSDIAVEWE |
| | | | | SNGQPENNYKTTPPVLDSDGSFFLYSKLTVDKS |
| | | | | RWQQGNVFSCSVMHEALHNHYTQKSLSLSP |
| 19. | Fc monomer-3- | artificial | Aa | DKTHTCPPCPAPELLGGPSVFLFPPKPKDTLMIS |
| | c/+g | | | RTPEVTCVVVDVSHEDPEVKFNWYVDGVEVH |
| | | | | NAKTKPREEQYNSTYRVVSVLTVLHQDWLNG |
| | | | | KEYKCKVSNKALPAPIEKTISKAKGQPREPQVY |
| | | | | TLPPSREEMTKNQVSLTCLVKGFYPSDIAVEWE |
| | | | | SNGQPENNYKTTPPVLDSDGSFFLYSKLTVDKS |
| | | | | RWQQGNVFSCSVMHEALHNHYTQKSLSLSPG |
| | | | | K |
| 20. | Fc monomer-4- | artificial | Aa | DKTHTCPPCPAPELLGGPSVFLFPPKPKDTLMIS |
| | c/+g/delGK | | | RTPEVTCVVVDVSHEDPEVKFNWYVDGVEVH |
| | | | | NAKTKPREEQYNSTYRVVSVLTVLHQDWLNG |
| | | | | KEYKCKVSNKALPAPIEKTISKAKGQPREPQVY |
| | | | | TLPPSREEMTKNQVSLTCLVKGFYPSDIAVEWE |
| | | | | SNGQPENNYKTTPPVLDSDGSFFLYSKLTVDKS |
| | | | | RWQQGNVFSCSVMHEALHNHYTQKSLSLSP |
| 21. | Fc monomer-5- | artificial | Aa | DKTHTCPPCPAPELLGGPSVFLFPPKPKDTLMIS |
| | c/+g | | | RTPEVTCVVVDVSHEDPEVKFNWYVDGVEVH |
| | | | | NAKTKPREEQYGSTYRVVSVLTVLHQDWLNG |
| | | | | KEYKCKVSNKALPAPIEKTISKAKGQPREPQVY |
| | | | | TLPPSREEMTKNQVSLTCLVKGFYPSDIAVEWE |
| | | | | SNGQPENNYKTTPPVLDSDGSFFLYSKLTVDKS |
| | | | | RWQQGNVFSCSVMHEALHNHYTQKSLSLSPG |
| | | | | K |
| 22. | Fc monomer-6- | artificial | Aa | DKTHTCPPCPAPELLGGPSVFLFPPKPKDTLMIS |
| | c/+g/delGK | | | RTPEVTCVVVDVSHEDPEVKFNWYVDGVEVH |
| | | | | NAKTKPREEQYGSTYRVVSVLTVLHQDWLNG |
| | | | | KEYKCKVSNKALPAPIEKTISKAKGQPREPQVY |
| | | | | TLPPSREEMTKNQVSLTCLVKGFYPSDIAVEWE |
| | | | | SNGQPENNYKTTPPVLDSDGSFFLYSKLTVDKS |
| | | | | RWQQGNVFSCSVMHEALHNHYTQKSLSLSP |
| 23. | Fc monomer-7- | artificial | Aa | DKTHTCPPCPAPELLGGPSVFLFPPKPKDTLMIS |
| | c/+g | | | RTPEVTCVVVDVSHEDPEVKFNWYVDGVEVH |
| | | | | NAKTKPCEEQYNSTYRCVSVLTVLHQDWLNG |
| | | | | KEYKCKVSNKALPAPIEKTISKAKGQPREPQVY |
| | | | | TLPPSREEMTKNQVSLTCLVKGFYPSDIAVEWE |
| | | | | SNGQPENNYKTTPPVLDSDGSFFLYSKLTVDKS |
| | | | | RWQQGNVFSCSVMHEALHNHYTQKSLSLSPG |
| | | | | K |
| 24. | Fc monomer-8- | artificial | aa | DKTHTCPPCPAPELLGGPSVFLFPPKPKDTLMIS |
| | c/+g/delGK | | | RTPEVTCVVVDVSHEDPEVKFNWYVDGVEVH |
| | | | | NAKTKPCEEQYNSTYRCVSVLTVLHQDWLNG |
| | | | | KEYKCKVSNKALPAPIEKTISKAKGQPREPQVY |
| | | | | TLPPSREEMTKNQVSLTCLVKGFYPSDIAVEWE |
| | | | | SNGQPENNYKTTPPVLDSDGSFFLYSKLTVDKS |
| | | | | RWQQGNVFSCSVMHEALHNHYTQKSLSLSP |
| 25. | scFc - Spacer | artificial | aa | DKTHTCPPCPAPELLGGPSVFLFPPKPKDTLMIS |
| | | | | RTPEVTCVVVDVSHEDPEVKFNWYVDGVEVH |
| | | | | NAKTKPCEEQYGSTYRCVSVLTVLHQDWLNG |
| | | | | KEYKCKVSNKALPAPIEKTISKAKGQPREPQVY |
| | | | | TLPPSREEMTKNQVSLTCLVKGFYPSDIAVEWE |
| | | | | SNGQPENNYKTTPPVLDSDGSFFLYSKLTVDKS |
| | | | | RWQQGNVFSCSVMHEALHNHYTQKSLSLSPG |
| | | | | KGGGGSGGGGSGGGGSGGGGSGGGGSGGGGS |
| | | | | DKTHTCPPCPAPELLGGPSVFLFPPKPKDTLMIS |
| | | | | RTPEVTCVVVDVSHEDPEVKFNWYVDGVEVH |
| | | | | NAKTKPCEEQYGSTYRCVSVLTVLHQDWLNG |
| | | | | KEYKCKVSNKALPAPIEKTISKAKGQPREPQVY |
| | | | | TLPPSREEMTKNQVSLTCLVKGFYPSDIAVEWE |
| | | | | SNGQPENNYKTTPPVLDSDGSFFLYSKLTVDKS |
| | | | | RWQQGNVFSCSVMHEALHNHYTQKSLSLSPG |
| | | | | K |
| 26. | scFc-2 Spacer | artificial | aa | DKTHTCPPCPAPELLGGPSVFLFPPKPKDTLMIS |
| | | | | RTPEVTCVVVDVSHEDPEVKFNWYVDGVEVH |
| | | | | NAKTKPCEEQYGSTYRCVSVLTVLHQDWLNG |
| | | | | KEYKCKVSNKALPAPIEKTISKAKGQPREPQVY |
| | | | | TLPPSREEMTKNQVSLTCLVKGFYPSDIAVEWE |
| | | | | SNGQPENNYKTTPPVLDSDGSFFLYSKLTVDKS |
| | | | | RWQQGNVFSCSVMHEALHNHYTQKSLSLSPG |
| | | | | GGGSGGGGSGGGGSGGGGSGGGGSGGGGSDK |
| | | | | THTCPPCPAPELLGGPSVFLFPPKPKDTLMISRT |
| | | | | PEVTCVVVDVSHEDPEVKFNWYVDGVEVHNA |
| | | | | KTKPCEEQYGSTYRCVSVLTVLHQDWLNGKE |
| | | | | YKCKVSNKALPAPIEKTISKAKGQPREPQVYTL |
| | | | | PPSREEMTKNQVSLTCLVKGFYPSDIAVEWES |
| | | | | NGQPENNYKTTPPVLDSDGSFFLYSKLTVDKS |
| | | | | RWQQGNVFSCSVMHEALHNHYTQKSLSLSP |
| 27. | scFc-3 Spacer | artificial | aa | DKTHTCPPCPAPELLGGPSVFLFPPKPKDTLMIS |
| | | | | RTPEVTCVVVDVSHEDPEVKFNWYVDGVEVH |
| | | | | NAKTKPREEQYNSTYRVVSVLTVLHQDWLNG |
| | | | | KEYKCKVSNKALPAPIEKTISKAKGQPREPQVY |
| | | | | TLPPSREEMTKNQVSLTCLVKGFYPSDIAVEWE |
| | | | | SNGQPENNYKTTPPVLDSDGSFFLYSKLTVDKS |
| | | | | RWQQGNVFSCSVMHEALHNHYTQKSLSLSPG |
| | | | | KGGGGSGGGGSGGGGSGGGGSGGGGSGGGGS |
| | | | | DKTHTCPPCPAPELLGGPSVFLFPPKPKDTLMIS |
| | | | | RTPEVTCVVVDVSHEDPEVKFNWYVDGVEVH |
| | | | | NAKTKPREEQYNSTYRVVSVLTVLHQDWLNG |
| | | | | KEYKCKVSNKALPAPIEKTISKAKGQPREPQVY |
| | | | | TLPPSREEMTKNQVSLTCLVKGFYPSDIAVEWE |
| | | | | SNGQPENNYKTTPPVLDSDGSFFLYSKLTVDKS |
| | | | | RWQQGNVFSCSVMHEALHNHYTQKSLSLSPG |
| | | | | K |
| 28. | scFc-4 Spacer | artificial | aa | DKTHTCPPCPAPELLGGPSVFLFPPKPKDTLMIS |
| | | | | RTPEVTCVVVDVSHEDPEVKFNWYVDGVEVH |
| | | | | NAKTKPREEQYNSTYRVVSVLTVLHQDWLNG |
| | | | | KEYKCKVSNKALPAPIEKTISKAKGQPREPQVY |
| | | | | TLPPSREEMTKNQVSLTCLVKGFYPSDIAVEWE |
| | | | | SNGQPENNYKTTPPVLDSDGSFFLYSKLTVDKS |
| | | | | RWQQGNVFSCSVMHEALHNHYTQKSLSLSPG |
| | | | | GGGSGGGGSGGGGSGGGGSGGGGSGGGGSDK |
| | | | | THTCPPCPAPELLGGPSVFLFPPKPKDTLMISRT |
| | | | | PEVTCVVVDVSHEDPEVKFNWYVDGVEVHNA |
| | | | | KTKPREEQYNSTYRVVSVLTVLHQDWLNGKE |
| | | | | YKCKVSNKALPAPIEKTISKAKGQPREPQVYTL |
| | | | | PPSREEMTKNQVSLTCLVKGFYPSDIAVEWES |
| | | | | NGQPENNYKTTPPVLDSDGSFFLYSKLTVDKS |
| | | | | RWQQGNVFSCSVMHEALHNHYTQKSLSLSP |
| 29. | scFc-5 Spacer | artificial | aa | DKTHTCPPCPAPELLGGPSVFLFPPKPKDTLMIS |
| | | | | RTPEVTCVVVDVSHEDPEVKFNWYVDGVEVH |
| | | | | NAKTKPREEQYGSTYRVVSVLTVLHQDWLNG |
| | | | | KEYKCKVSNKALPAPIEKTISKAKGQPREPQVY |
| | | | | TLPPSREEMTKNQVSLTCLVKGFYPSDIAVEWE |
| | | | | SNGQPENNYKTTPPVLDSDGSFFLYSKLTVDKS |
| | | | | RWQQGNVFSCSVMHEALHNHYTQKSLSLSPG |
| | | | | KGGGGSGGGGSGGGGSGGGGSGGGGSGGGGS |
| | | | | DKTHTCPPCPAPELLGGPSVFLFPPKPKDTLMIS |
| | | | | RTPEVTCVVVDVSHEDPEVKFNWYVDGVEVH |
| | | | | NAKTKPREEQYGSTYRVVSVLTVLHQDWLNG |
| | | | | KEYKCKVSNKALPAPIEKTISKAKGQPREPQVY |
| | | | | TLPPSREEMTKNQVSLTCLVKGFYPSDIAVEWE |
| | | | | SNGQPENNYKTTPPVLDSDGSFFLYSKLTVDKS |
| | | | | RWQQGNVFSCSVMHEALHNHYTQKSLSLSPG |
| | | | | K |
| 30. | scFc-6 Spacer | artificial | aa | DKTHTCPPCPAPELLGGPSVFLFPPKPKDTLMIS |
| | | | | RTPEVTCVVVDVSHEDPEVKFNWYVDGVEVH |
| | | | | NAKTKPREEQYGSTYRVVSVLTVLHQDWLNG |
| | | | | KEYKCKVSNKALPAPIEKTISKAKGQPREPQVY |
| | | | | TLPPSREEMTKNQVSLTCLVKGFYPSDIAVEWE |
| | | | | SNGQPENNYKTTPPVLDSDGSFFLYSKLTVDKS |
| | | | | RWQQGNVFSCSVMHEALHNHYTQKSLSLSPG |
| | | | | GGGSGGGGSGGGGSGGGGSGGGGSGGGGSDK |
| | | | | THTCPPCPAPELLGGPSVFLFPPKPKDTLMISRT |
| | | | | PEVTCVVVDVSHEDPEVKFNWYVDGVEVHNA |
| | | | | KTKPREEQYGSTYRVVSVLTVLHQDWLNGKE |
| | | | | YKCKVSNKALPAPIEKTISKAKGQPREPQVYTL |
| | | | | PPSREEMTKNQVSLTCLVKGFYPSDIAVEWES |
| | | | | NGQPENNYKTTPPVLDSDGSFFLYSKLTVDKS |
| | | | | RWQQGNVFSCSVMHEALHNHYTQKSLSLSP |
| 31. | scFc-7 Spacer | artificial | aa | DKTHTCPPCPAPELLGGPSVFLFPPKPKDTLMIS |
| | | | | RTPEVTCVVVDVSHEDPEVKFNWYVDGVEVH |
| | | | | NAKTKPCEEQYNSTYRCVSVLTVLHQDWLNG |
| | | | | KEYKCKVSNKALPAPIEKTISKAKGQPREPQVY |
| | | | | TLPPSREEMTKNQVSLTCLVKGFYPSDIAVEWE |
| | | | | SNGQPENNYKTTPPVLDSDGSFFLYSKLTVDKS |
| | | | | RWQQGNVFSCSVMHEALHNHYTQKSLSLSPG |
| | | | | KGGGGSGGGGSGGGGSGGGGSGGGGSGGGGS |
| | | | | DKTHTCPPCPAPELLGGPSVFLFPPKPKDTLMIS |
| | | | | RTPEVTCVVVDVSHEDPEVKFNWYVDGVEVH |
| | | | | NAKTKPCEEQYNSTYRCVSVLTVLHQDWLNG |
| | | | | KEYKCKVSNKALPAPIEKTISKAKGQPREPQVY |
| | | | | TLPPSREEMTKNQVSLTCLVKGFYPSDIAVEWE |
| | | | | SNGQPENNYKTTPPVLDSDGSFFLYSKLTVDKS |
| | | | | RWQQGNVFSCSVMHEALHNHYTQKSLSLSPG |
| | | | | K |
| 32. | scFc-8 Spacer | artificial | Aa | DKTHTCPPCPAPELLGGPSVFLFPPKPKDTLMIS |
| | | | | RTPEVTCVVVDVSHEDPEVKFNWYVDGVEVH |
| | | | | NAKTKPCEEQYNSTYRCVSVLTVLHQDWLNG |
| | | | | KEYKCKVSNKALPAPIEKTISKAKGQPREPQVY |
| | | | | TLPPSREEMTKNQVSLTCLVKGFYPSDIAVEWE |
| | | | | SNGQPENNYKTTPPVLDSDGSFFLYSKLTVDKS |
| | | | | RWQQGNVFSCSVMHEALHNHYTQKSLSLSPG |
| | | | | GGGSGGGGSGGGGSGGGGSGGGGSGGGGSDK |
| | | | | THTCPPCPAPELLGGPSVFLFPPKPKDTLMISRT |
| | | | | PEVTCVVVDVSHEDPEVKFNWYVDGVEVHNA |
| | | | | KTKPCEEQYNSTYRCVSVLTVLHQDWLNGKE |
| | | | | YKCKVSNKALPAPIEKTISKAKGQPREPQVYTL |
| | | | | PPSREEMTKNQVSLTCLVKGFYPSDIAVEWES |
| | | | | NGQPENNYKTTPPVLDSDGSFFLYSKLTVDKS |
| | | | | RWQQGNVFSCSVMHEALHNHYTQKSLSLSP |
| 33. | scFc_mod_GQ_cli | artificial | Aa | CPPCPAPELLGGPSVFLFPPKPKDTLMISRTPEV |
| | ppingvariant - | | | TCVVVDVSHEEPEVKFNWYVDGVEVHNAKTK |
| | Spacer | | | PCEEQYGSTYRCVSVLTVLHQDWLNGKEYKC |
| | | | | KVSNKALPAPIEKTISKAKGQPREPQVYTLPPS |
| | | | | REEMTKNQVSLTCLVKGFYPSDIAVEWESNGQ |
| | | | | PENNYKTTPPVLDSDGSFFLYSKLTVDKSRWQ |
| | | | | QGNVFSCSVMHEALHNHYTQKSLSLSPGKGG |
| | | | | GGQGGGGQGGGGQGGGGQGGGGQGGGGQCP |
| | | | | PCPAPELLGGPSVFLFPPKPKDTLMISRTPEVTC |
| | | | | VVVDVSHEEPEVKFNWYVDGVEVHNAKTKPC |
| | | | | EEQYGSTYRCVSVLTVLHQDWLNGKEYKCKV |
| | | | | SNKALPAPIEKTISKAKGQPREPQVYTLPPSREE |
| | | | | MTKNQVSLTCLVKGFYPSDIAVEWESNGQPEN |
| | | | | NYKTTPPVLDSDGSFFLYSKLTVDKSRWQQGN |
| | | | | VFSCSVMHEALHNHYTQKSLSLSPGK |
| 34. | 2x scFc - double | artificial | Aa | DKTHTCPPCPAPELLGGPSVFLFPPKPKDTLMIS |
| | size Spacer | | | RTPEVTCVVVDVSHEDPEVKFNWYVDGVEVH |
| | | | | NAKTKPCEEQYGSTYRCVSVLTVLHQDWLNG |
| | | | | KEYKCKVSNKALPAPIEKTISKAKGQPREPQVY |
| | | | | TLPPSREEMTKNQVSLTCLVKGFYPSDIAVEWE |
| | | | | SNGQPENNYKTTPPVLDSDGSFFLYSKLTVDKS |
| | | | | RWQQGNVFSCSVMHEALHNHYTQKSLSLSPG |
| | | | | KGGGGSGGGGSGGGGSGGGGSGGGGSGGGGS |
| | | | | DKTHTCPPCPAPELLGGPSVFLFPPKPKDTLMIS |
| | | | | RTPEVTCVVVDVSHEDPEVKFNWYVDGVEVH |
| | | | | NAKTKPCEEQYGSTYRCVSVLTVLHQDWLNG |
| | | | | KEYKCKVSNKALPAPIEKTISKAKGQPREPQVY |
| | | | | TLPPSREEMTKNQVSLTCLVKGFYPSDIAVEWE |
| | | | | SNGQPENNYKTTPPVLDSDGSFFLYSKLTVDKS |
| | | | | RWQQGNVFSCSVMHEALHNHYTQKSLSLSPG |
| | | | | KDKTHTCPPCPAPELLGGPSVFLFPPKPKDTLM |
| | | | | ISRTPEVTCVVVDVSHEDPEVKFNWYVDGVEV |
| | | | | HNAKTKPCEEQYGSTYRCVSVLTVLHQDWLN |
| | | | | GKEYKCKVSNKALPAPIEKTISKAKGQPREPQV |
| | | | | YTLPPSREEMTKNQVSLTCLVKGFYPSDIAVE |
| | | | | WESNGQPENNYKTTPPVLDSDGSFFLYSKLTV |
| | | | | DKSRWQQGNVFSCSVMHEALHNHYTQKSLSL |
| | | | | SPGKGGGGSGGGGSGGGGSGGGGSGGGGSGG |
| | | | | GGSDKTHTCPPCPAPELLGGPSVFLFPPKPKDT |
| | | | | LMISRTPEVTCVVVDVSHEDPEVKFNWYVDG |
| | | | | VEVHNAKTKPCEEQYGSTYRCVSVLTVLHQD |
| | | | | WLNGKEYKCKVSNKALPAPIEKTISKAKGQPR |
| | | | | EPQVYTLPPSREEMTKNQVSLTCLVKGFYPSDI |
| | | | | AVEWESNGQPENNYKTTPPVLDSDGSFFLYSK |
| | | | | LTVDKSRWQQGNVFSCSVMHEALHNHYTQKS |
| | | | | LSLSPGK |
| 35. | heteroFc (A) - | artificial | Aa | DKTHTCPPCPAPELLGGPSVFLFPPKPKDTLMIS |
| | Spacer | | | RTPEVTCVVVDVSHEDPEVKFNWYVDGVEVH |
| | | | | NAKTKPCEEQYGSTYRCVSVLTVLHQDWLNG |
| | | | | KEYKCKVSNKALPAPIEKTISKAKGQPREPQVY |
| | | | | TLPPSREEMTKNQVSLTCLVKGFYPSDIAVEWE |
| | | | | SNGQPENNYDTTPPVLDSDGSFFLYSDLTVDKS |
| | | | | RWQQGNVFSCSVMHEALHNHYTQDSLSLSPG |
| | | | | K |
| 36. | heteroFc (B) - | artificial | Aa | DKTHTCPPCPAPELLGGPSVFLFPPKPKDTLMIS |
| | Spacer | | | RTPEVTCVVVDVSHEDPEVKFNWYVDGVEVH |
| | | | | NAKTKPCEEQYGSTYRCVSVLTVLHQDWLNG |
| | | | | KEYKCKVSNKALPAPIEKTISKAKGQPREPQVY |
| | | | | TLPPSRKEMTKNQVSLTCLVKGFYPSDIAVEW |
| | | | | ESNGQPENNYKTTPPVLKSDGSFFLYSKLTVDK |
| | | | | SRWQQGNVFSCSVMHEALHNHYTQKSLSLSP |
| | | | | GK |
| 37. | I2C - HCDR1 | artificial | Aa | KYAMN |
| 38. | I2C - HCDR2 | artificial | Aa | RIRSKYNNYATYYADSVKD |
| 39. | I2C - HCDR3 | artificial | Aa | HGNFGNSYISYWAY |
| 40. | I2C - LCDR1 | artificial | Aa | GSSTGAVTSGNYPN |
| 41. | I2C - LCDR2 | artificial | aa | GTKFLAP |
| 42. | I2C - LCDR3 | artificial | aa | VLWYSNRWV |
| 43. | I2C - VH | artificial | aa | EVQLVESGGGLVQPGGSLKLSCAASGFTFNKY |
| | | | | AMNWVRQAPGKGLEWVARIRSKYNNYATYY |
| | | | | ADSVKDRFTISRDDSKNTAYLQMNNLKTEDTA |
| | | | | VYYCVRHGNFGNSYISYWAYWGQGTLVTVSS |
| 44. | I2C - VL | artificial | aa | QTVVTQEPSLTVSPGGTVTLTCGSSTGAVTSGN |
| | | | | YPNWVQQKPGQAPRGLIGGTKFLAPGTPARFS |
| | | | | GSLLGGKAALTLSGVQPEDEAEYYCVLWYSN |
| | | | | RWVFGGGTKLTVL |
| 45. | I2C_44/100cc - | artificial | aa | KYAMN |
| | HCDR1 | | | |
| 46. | I2C_44/100cc - | artificial | aa | RIRSKYNNYATYYADSVKD |
| | HCDR2 | | | |
| 47. | I2C_44/100cc - | artificial | aa | HGNFGNSYISYWAY |
| | HCDR3 | | | |
| 48. | I2C_44/100cc - | artificial | aa | GSSTGAVTSGNYPN |
| | LCDR1 | | | |
| 49. | I2C_44/100cc - | artificial | Aa | GTKFLAP |
| | LCDR2 | | | |
| 50. | I2C_44/100cc - | artificial | Aa | VLWYSNRWV |
| | LCDR3 | | | |
| 51. | I2C_44/100cc - | artificial | Aa | EVQLVESGGGLVQPGGSLKLSCAASGFTFNKY |
| | VH | | | AMNWVRQAPGKCLEWVARIRSKYNNYATYY |
| | | | | ADSVKDRFTISRDDSKNTAYLQMNNLKTEDTA |
| | | | | VYYCVRHGNFGNSYISYWAYWGQGTLVTVSS |
| 52. | I2C_44/100cc - | artificial | Aa | QTVVTQEPSLTVSPGGTVTLTCGSSTGAVTSGN |
| | VL | | | YPNWVQQKPGQAPRGLIGGTKFLAPGTPARFS |
| | | | | GSLLGGKAALTLSGVQPEDEAEYYCVLWYSN |
| | | | | RWVFGCGTKLTVL |
| 53. | I2E - HCDR1 | artificial | Aa | KYAIN |
| 54. | I2E - HCDR2 | artificial | Aa | RIRSKYNNYATYYADAVKD |
| 55. | I2E - HCDR3 | artificial | Aa | AGNFGSSYISYWAY |
| 56. | I2E - LCDR1 | artificial | Aa | GSSTGAVTSGNYPN |
| 57. | I2E - LCDR2 | artificial | Aa | GTKFLAP |
| 58. | I2E - LCDR3 | artificial | aa | VLWYSNRWV |
| 59. | I2E - VH | artificial | aa | EVQLVESGGGLVQPGGSLKLSCAASGFTFNKY |
| | | | | AINWVRQAPGKGLEWVARIRSKYNNYATYYA |
| | | | | DAVKDRFTISRDDSKNTVYLQMNNLKTEDTA |
| | | | | VYYCARAGNFGSSYISYWAYWGQGTLVTVSS |
| 60. | I2E - VL | artificial | aa | QTVVTQEPSLTVSPGGTVTITCGSSTGAVTSGN |
| | | | | YPNWVQKKPGQAPRGLIGGTKFLAPGTPARFS |
| | | | | GSLSGGKAALTLSGVQPEDEAEYYCVLWYSN |
| | | | | RWVFGSGTKLTVL |
| 61. | I2L - HCDR1 | artificial | aa | KYAMN |
| 62. | I2L - HCDR2 | artificial | aa | RIRSKYNNYATYYADAVKD |
| 63. | I2L - HCDR3 | artificial | aa | AGNFGSSYISYFAY |
| 64. | I2L - LCDR1 | artificial | aa | GSSTGAVTSGNYPN |
| 65. | I2L - LCDR2 | artificial | aa | GTKFLAP |
| 66. | I2L - LCDR3 | artificial | Aa | VLYYSNRWV |
| 67. | I2L - VH | artificial | Aa | EVQLVESGGGLVQPGGSLKLSCAASGFTFNKY |
| | | | | AMNWVRQAPGKGMEWVARIRSKYNNYATYY |
| | | | | ADAVKDRFTISRDDSKNTLYLQMNNLKTEDTA |
| | | | | VYYCVRAGNFGSSYISYFAYWGQGTLVTVSS |
| 68. | I2L - VL | artificial | Aa | QTVVTQEPSLTVSPGGTVTITCGSSTGAVTSGN |
| | | | | YPNWIQKKPGQAPRGLIGGTKFLAPGTPARFSG |
| | | | | SLEGGKAALTLSGVQPEDEAEYYCVLYYSNR |
| | | | | WVFGSGTKLTVL |
| 69. | I2M2 - HCDR1 | artificial | Aa | KYAIN |
| 70. | I2M2 - HCDR2 | artificial | Aa | RIRSKYNNYATYYADAVKD |
| 71. | I2M2 - HCDR3 | artificial | Aa | NANFGTSYISYFAY |
| 72. | I2M2 - LCDR1 | artificial | Aa | GSSTGAVTSGNYPN |
| 73. | I2M2 - LCDR2 | artificial | Aa | GTKFLAP |
| 74. | I2M2 - LCDR3 | artificial | Aa | VLWYSNRWV |
| 75. | I2M2 - VH | artificial | aa | EVQLVESGGGLVQPGGSLKLSCAASGFTFNKY |
| | | | | AINWVREAPGKGLEWVARIRSKYNNYATYYA |
| | | | | DAVKDRFTISRDDSKNTAYLQMNNLKTEDTA |
| | | | | VYYCVRNANFGTSYISYFAYWGQGTLVTVSS |
| 76. | I2M2 - VL | artificial | aa | QTVVTQEPSLTVSPGGTVTLTCGSSTGAVTSGN |
| | | | | YPNWVQKKPGQAPRGLIGGTKFLAPGTPARFS |
| | | | | GSLLGGKAALTLSGVQPEDEAEYYCVLWYSN |
| | | | | RWVFGSGTKLTVL |
| 77. | MS 01-G11 CC - | artificial | aa | DYYMT |
| | HCDR1 | | | |
| 78. | MS 01-G11 CC - | artificial | aa | YISSSGSTIYYAEAVKG |
| | HCDR2 | | | |
| 79. | MS 01-G11 CC - | artificial | aa | DRNSHFDY |
| | HCDR3 | | | |
| 80. | MS 01-G11 CC - | artificial | aa | RASQGIRTWLA |
| | LCDR1 | | | |
| 81. | MS 01-G11 CC - | artificial | aa | GASGLQS |
| | LCDR2 | | | |
| 82. | MS 01-G11 CC - | artificial | aa | QQAESFPRT |
| | LCDR3 | | | |
| 83. | MS 01-G11 CC - | artificial | Aa | QVQLVESGGGLVKPGGSLRLSCAASGFTFSDY |
| | VH | | | YMTWIRQAPGKCLEWLSYISSSGSTIYYAEAV |
| | | | | KGRFTISRDNAKNSLFLQMNSLRAEDTAVYYC |
| | | | | ARDRNSHFDYWGQGTLVTVSS |
| 84. | MS 01-G11 CC - | artificial | Aa | DIMTQSPSSVSASVGDRVTITCRASQGIRTWLA |
| | VL | | | WYQQKPGKAPKLLIYGASGLQSGVPSRFSGSG |
| | | | | SGTDFTLTISSLQPEDFATYYCQQAESFPRTFGC |
| | | | | GTKVEIK |
| 85. | MS 01-G11 CC EI - | artificial | Aa | EIMTQSPSSVSASVGDRVTITCRASQGIRTWLA |
| | VL | | | WYQQKPGKAPKLLIYGASGLQSGVPSRFSGSG |
| | | | | SGTDFTLTISSLQPEDFATYYCQQAESFPRTFGC |
| | | | | GTKVEIK |
| 86. | MS 15-B12 CC - | artificial | Aa | SSSYFWG |
| | HCDR1 | | | |
| 87. | MS 15-B12 CC - | artificial | Aa | NIYYSGSSNYNPSLKS |
| | HCDR2 | | | |
| 88. | MS 15-B12 CC - | artificial | Aa | LPRGDRDAFDI |
| | HCDR3 | | | |
| 89. | MS 15-B12 CC - | artificial | Aa | RASQGISNYLA |
| | LCDR1 | | | |
| 90. | MS 15-B12 CC - | artificial | Aa | AASTLQS |
| | LCDR2 | | | |
| 91. | MS 15-B12 CC - | artificial | Aa | QQSYSTPFT |
| | LCDR3 | | | |
| 92. | MS 15-B12 CC - | artificial | aa | QVQLQESGPGLVKPSETLSLTCTVSGGSISSSSY |
| | VH | | | FWGWIRQPPGKCLEWIGNIYYSGSSNYNPSLKS |
| | | | | RVTISVDTSKNQFSLKLSSVTAADTAVYYCAR |
| | | | | LPRGDRDAFDIWGQGTMVTVSS |
| 93. | MS 15-B12 CC - | artificial | aa | DIVMTQSPSSLSASVGDRVTITCRASQGISNYL |
| | VL | | | AWYQQKPGKVPKLLIYAASTLQSGVPSRFSGS |
| | | | | GSGTDFTLTISSLQPEDFATYYCQQSYSTPFTFG |
| | | | | CGTKVEIK |
| 94. | MS 15-B12 CC EI - | artificial | aa | EIVMTQSPSSLSASVGDRVTITCRASQGISNYLA |
| | VL | | | WYQQKPGKVPKLLIYAASTLQSGVPSRESGSG |
| | | | | SGTDFTLTISSLQPEDFATYYCQQSYSTPFTFGC |
| | | | | GTKVEIK |
| 95. | MS 25-E3 CC - | artificial | aa | SSSYFWV |
| | HCDR1 | | | |
| 96. | MS 25-E3 CC - | artificial | aa | SIYYSGSTYYNPSLKS |
| | HCDR2 | | | |
| 97. | MS 25-E3 CC - | artificial | aa | LPRGDRMTFDI |
| | HCDR3 | | | |
| 98. | MS 25-E3 CC - | artificial | aa | RASQSVSSSYLA |
| | LCDR1 | | | |
| 99. | MS 25-E3 CC - | artificial | aa | GASSRAT |
| | LCDR2 | | | |
| 100. | MS 25-E3 CC - | artificial | Aa | QQYGSSPFT |
| | LCDR3 | | | |
| 101. | MS 25-E3 CC - | artificial | Aa | QVQLQESGPGLVKPSETLSLTCTVSGGSISSSSY |
| | VH | | | FWVWIRQPPGKCLEWIGSIYYSGSTYYNPSLKS |
| | | | | RVTISVDTSKNQFSLKLNSVTAADTAVYYCAR |
| | | | | LPRGDRMTFDIWGQGTMVTVSS |
| 102. | MS 25-E3 CC - | artificial | Aa | EIVLTQSPGTLSLSPGERATLSCRASQSVSSSYL |
| | VL | | | AWYQQKPGQAPRLLIYGASSRATGIPDRESGSG |
| | | | | SGTDFTLTISRLEPEDFAVYYCQQYGSSPFTFGC |
| | | | | GTKLEIK |
| 103. | MS 36-C5 CC - | artificial | Aa | SYAMS |
| | HCDR1 | | | |
| 104. | MS 36-C5 CC - | artificial | Aa | AISGSGEQWYYAPSVKG |
| | HCDR2 | | | |
| 105. | MS 36-C5 CC - | artificial | Aa | VRNYYGSGSLDY |
| | HCDR3 | | | |
| 106. | MS 36-C5 CC - | artificial | Aa | RASQSFSSAYLA |
| | LCDR1 | | | |
| 107. | MS 36-C5 CC - | artificial | Aa | GASIRAT |
| | LCDR2 | | | |
| 108. | MS 36-C5 CC - | artificial | Aa | QQYGSSLT |
| | LCDR3 | | | |
| 109. | MS 36-C5 CC - | artificial | aa | EVQLLESGGGVVQPGRSLRLSCAASGFTFSSYA |
| | VH | | | MSWVRQAPGKCLEWVSAISGSGEQWYYAPSV |
| | | | | KGRFTISRDNSKNTLYLQMNSLRAEDTAVYYC |
| | | | | AKVRNYYGSGSLDYWGQGTLVTVSS |
| 110. | MS 36-C5 CC - | artificial | aa | EIVLTQSPGTLSLSPGERATLSCRASQSFSSAYL |
| | VL | | | AWYQQKPGQAPRLLIYGASIRATGIPDRESGSG |
| | | | | SGTDFTLTISRLEPEDFAVYYCQQYGSSLTFGC |
| | | | | GTKVEIK |
| 111. | MS 36-G7 CC - | artificial | aa | SYAMS |
| | HCDR1 | | | |
| 112. | MS 36-G7 CC - | artificial | aa | AISGSGEGDYYANSVKG |
| | HCDR2 | | | |
| 113. | MS 36-G7 CC - | artificial | aa | VRNYYGSGSLDY |
| | HCDR3 | | | |
| 114. | MS 36-G7 CC - | artificial | aa | RASQSVSSTYLA |
| | LCDR1 | | | |
| 115. | MS 36-G7 CC - | artificial | aa | GASIRAT |
| | LCDR2 | | | |
| 116. | MS 36-G7 CC - | artificial | aa | QQYGSSLT |
| | LCDR3 | | | |
| 117. | MS 36-G7 CC - | artificial | Aa | EVQLLESGGGVVQPGRSLRLSCAASGFTFSSYA |
| | VH | | | MSWVRQAPGMCLEWVSAISGSGEGDYYANSV |
| | | | | KGRFTISRDNSKNTLYLQMNSLRAEDTAVYYC |
| | | | | AKVRNYYGSGSLDYWGQGTLVTVSS |
| 118. | MS 36-G7 CC - | artificial | Aa | EIVLTQSPGTLSLSPGERATLSCRASQSVSSTYL |
| | VL | | | AWYQQKPGQAPRLLIYGASIRATGIPDRESGSG |
| | | | | SGTDFTLTISRLEPEDFAVYYCQQYGSSLTFGC |
| | | | | GTKVEIK |
| 119. | MS 37-E5 CC - | artificial | Aa | SYAMS |
| | HCDR1 | | | |
| 120. | MS 37-E5 CC - | artificial | Aa | AISGSGGSTYYAIDVKG |
| | HCDR2 | | | |
| 121. | MS 37-E5 CC - | artificial | Aa | EGYYPGSGYPLYYYFGMDV |
| | HCDR3 | | | |
| 122. | MS 37-E5 CC - | artificial | Aa | RASQSVSSSYLA |
| | LCDR1 | | | |
| 123. | MS 37-E5 CC - | artificial | Aa | GASSRAT |
| | LCDR2 | | | |
| 124. | MS 37-E5 CC - | artificial | Aa | QQYGSSPIFT |
| | LCDR3 | | | |
| 125. | MS 37-E5 CC - | artificial | Aa | EVQLLESGGGLVQPGGSLRLSCAASGFTFSSYA |
| | VH | | | MSWVRQAPGKCLEWVSAISGSGGSTYYAIDV |
| | | | | KGRFTISRDNSKNTLYLQMNSLRAEDTAVYYC |
| | | | | AKEGYYPGSGYPLYYYFGMDVWGQGTTVTVS |
| | | | | S |
| 126. | MS 37-E5 CC - | artificial | aa | EIVLTQSPGTLSLSPGERATLSCRASQSVSSSYL |
| | VL | | | AWYQQKPGQAPRLLIYGASSRATGIPDRFSGSG |
| | | | | SGTDFTLTISRLEPEDFAVYYCQQYGSSPIFTFG |
| | | | | CGTKVEIK |
| 127. | MS 46-A3 CC - | artificial | aa | SYGMG |
| | HCDR1 | | | |
| 128. | MS 46-A3 CC - | artificial | aa | VISYHGSNKYYADAVKG |
| | HCDR2 | | | |
| 129. | MS 46-A3 CC - | artificial | aa | EGAHFGSGSYYPLYYYYAMDV |
| | HCDR3 | | | |
| 130. | MS 46-A3 CC - | artificial | aa | RASQSVSSSYLA |
| | LCDR1 | | | |
| 131. | MS 46-A3 CC - | artificial | aa | GASIRAT |
| | LCDR2 | | | |
| 132. | MS 46-A3 CC - | artificial | aa | QQTGSSPIFT |
| | LCDR3 | | | |
| 133. | MS 46-A3 CC - | artificial | aa | QVQLVESGGGVVQPGRSLRLSCAASGFTFSSY |
| | VH | | | GMGWVRQAPGKCLEWVAVISYHGSNKYYAD |
| | | | | AVKGRFTISRDNSKNTLYLQMNSLRAEDTAVY |
| | | | | YCAREGAHFGSGSYYPLYYYYAMDVWGQGT |
| | | | | TVTVSS |
| 134. | MS 46-A3 CC - | artificial | Aa | EIVTQSPGTLSLSPGERATLSCRASQSVSSSYLA |
| | VL | | | WYQQKPGQAPRLLIYGASIRATGIPDRFSGSGS |
| | | | | GTDFTLTISRLEPEDFAVYYCQQTGSSPIFTFGC |
| | | | | GTKVEIK |
| 135. | MS R195L CC - | artificial | Aa | SYAMS |
| | HCDR1 | | | |
| 136. | MS R195L CC - | artificial | Aa | AISGSGEFSYYAAAVKG |
| | HCDR2 | | | |
| 137. | MS R195L CC - | artificial | Aa | VRNYYGSGSLDY |
| | HCDR3 | | | |
| 138. | MS R195L CC - | artificial | Aa | RASQSVSSTYLA |
| | LCDR1 | | | |
| 139. | MS R195L CC - | artificial | Aa | GASIRAT |
| | LCDR2 | | | |
| 140. | MS R195L CC - | artificial | Aa | QQYQSSLT |
| | LCDR3 | | | |
| 141. | MS R195L CC - | artificial | Aa | EVQLLESGGGVVQPGRSLRLSCAASGFTFSSYA |
| | VH | | | MSWVRQAPGKCLEWVSAISGSGEFSYYAAAV |
| | | | | KGRFTISRDNSKNTLYLQMNSLRAEDTAVYYC |
| | | | | AKVRNYYGSGSLDYWGQGTLVTVSS |
| 142. | MS R195L CC - | artificial | Aa | EIVLTQSPGTLSLSPGERATLSCRASQSVSSTYL |
| | VL | | | AWYQQKPGQAPRLLIYGASIRATGIPDRESGSG |
| | | | | SGTDFTLTISRLEPEDFAVYYCQQYQSSLTFGC |
| | | | | GTKVEIK |
| 143. | MS R4L CC - | artificial | aa | GYYIH |
| | HCDR1 | | | |
| 144. | MS R4L CC - | artificial | aa | WINPNSGGTNYAQKFQG |
| | HCDR2 | | | |
| 145. | MS R4L CC - | artificial | aa | VEAVAGREYYYFSGMDV |
| | HCDR3 | | | |
| 146. | MS R4L CC - | artificial | aa | SGEKLGDKYVY |
| | LCDR1 | | | |
| 147. | MS R4L CC - | artificial | aa | QSTKRPS |
| | LCDR2 | | | |
| 148. | MS R4L CC - | artificial | aa | QAYHASTAV |
| | LCDR3 | | | |
| 149. | MS R4L CC - VH | artificial | aa | QVQLVQSGAEVKKPGASVKVSCKASGYTFTG |
| | | | | YYIHWVRQAPGQCLEWMGWINPNSGGTNYA |
| | | | | QKFQGRVTMTRDTSISTAYMELSRLRSDDTAV |
| | | | | YYCARVEAVAGREYYYFSGMDVWGQGTTVT |
| | | | | VSS |
| 150. | MS R4L CC - VL | artificial | aa | SYELTQPPSVSVSPGQTASITCSGEKLGDKYVY |
| | | | | WYQQKPGQSPVLVIYQSTKRPSGVPERFSGSNS |
| | | | | GNTATLTISGTQAMDEADYYCQAYHASTAVF |
| | | | | GCGTKLTVL |
| 151. | MS H2 - HCDR1 | artificial | Aa | SYGMG |
| 152. | MS H2 - HCDR2 | artificial | Aa | VISYDGSNKYYADSVKG |
| 153. | MS H2 - HCDR3 | artificial | Aa | EGAHFGSGSYYPLYYYYAMDV |
| 154. | MS H2 - LCDR1 | artificial | Aa | RASQSVSSSYLA |
| 155. | MS H2 - LCDR2 | artificial | Aa | GASIRAT |
| 156. | MS H2 - LCDR3 | artificial | Aa | QQYGSSPIFT |
| 157. | MS H2 - VH | artificial | Aa | EVQLLESGGGVVQPGRSLRLSCAASGFTFSSYG |
| | | | | MGWVRQAPGKGLEWVAVISYDGSNKYYADS |
| | | | | VKGRFTISRDNSKNTLYLQMNSLRAEDTAVYY |
| | | | | CAREGAHFGSGSYYPLYYYYAMDVWGQGTT |
| | | | | VTVSS |
| 158. | MS H2 - VL | artificial | Aa | ELTLTQSPGTLSLSPGERATLSCRASQSVSSSYL |
| | | | | AWYQQKPGQAPRLLIYGASIRATGIPDRESGSG |
| | | | | SGTDFTLTISRLEPEDFAVYYCQQYGSSPIFTFG |
| | | | | PGTKVEIK |
| 159. | CH3 005-D5 CC - | artificial | Aa | SYPIN |
| | HCDR1 | | | |
| 160. | CH3 005-D5 CC - | artificial | aa | VIWTGGGTNYASSVKG |
| | HCDR2 | | | |
| 161. | CH3 005-D5 CC - | artificial | aa | SRGVYDFKGRGAMDY |
| | HCDR3 | | | |
| 162. | CH3 005-D5 CC - | artificial | aa | KSSQSLLYSSNQKNYFA |
| | LCDR1 | | | |
| 163. | CH3 005-D5 CC - | artificial | aa | WASTRES |
| | LCDR2 | | | |
| 164. | CH3 005-D5 CC - | artificial | aa | QQYYSYPYT |
| | LCDR3 | | | |
| 165. | CH3 005-D5 CC - | artificial | aa | EVQLLESGGGLVQPGGSLRLSCAASGFSFSSYPI |
| | VH | | | NWVRQAPGKCLEWVGVIWTGGGTNYASSVK |
| | | | | GRFTISRDNSKNTVYLQMNSLRAEDTAVYYCA |
| | | | | KSRGVYDFKGRGAMDYWGQGTLVTVSS |
| 166. | CH3 005-D5 CC - | artificial | aa | DIVMTQSPDSLAVSLGERATINCKSSQSLLYSS |
| | VL | | | NQKNYFAWYQQKPGQPPKLLIYWASTRESGV |
| | | | | PDRFSGSGSGTDFTLTISSLQAEDVAVYYCQQY |
| | | | | YSYPYTFGCGTKLEIK |
| 167. | CH3 005-D5 CC | artificial | aa | EIVMTQSPDSLAVSLGERATINCKSSQSLLYSSN |
| | EI - VL | | | QKNYFAWYQQKPGQPPKLLIYWASTRESGVP |
| | | | | DRFSGSGSGTDFTLTISSLQAEDVAVYYCQQY |
| | | | | YSYPYTFGCGTKLEIK |
| 168. | CH3 03-C8 CC - | artificial | Aa | SYWMH |
| | HCDR1 | | | |
| 169. | CH3 03-C8 CC - | artificial | Aa | VISGSKSYTIYNQKVKG |
| | HCDR2 | | | |
| 170. | CH3 03-C8 CC - | artificial | Aa | SGPGYFDV |
| | HCDR3 | | | |
| 171. | CH3 03-C8 CC - | artificial | Aa | RASENIYSYLA |
| | LCDR1 | | | |
| 172. | CH3 03-C8 CC - | artificial | Aa | NAKTLAE |
| | LCDR2 | | | |
| 173. | CH3 03-C8 CC - | artificial | Aa | QHLNMTPYT |
| | LCDR3 | | | |
| 174. | CH3 03-C8 CC - | artificial | Aa | EVQLLESGGGLVQPGGSLRLSCAASGYTFSSY |
| | VH | | | WMHWVRQAPGKCLEWMGVISGSKSYTIYNQ |
| | | | | KVKGRFTISRDNSKNTVYLQMNSLRAGDTAV |
| | | | | YYCARSGPGYFDVWGQGTMVTVSS |
| 175. | CH3 03-C8 CC - | artificial | Aa | DIQLTQSPSFLSASVGDRVTITCRASENIYSYLA |
| | VL | | | WYQQKPGKAPKLLIYNAKTLAEGVPSRFSGSG |
| | | | | SGTEFTLTISSLQPEDFGTYYCQHLNMTPYTFG |
| | | | | CGTKLEIK |
| 176. | CH3 03-C8 CC | artificial | Aa | EIQLTQSPSFLSASVGDRVTITCRASENIYSYLA |
| | EI - VL | | | WYQQKPGKAPKLLIYNAKTLAEGVPSRFSGSG |
| | | | | SGTEFTLTISSLQPEDFGTYYCQHLNMTPYTFG |
| | | | | CGTKLEIK |
| 177. | CH3 08-A11 CC - | artificial | aa | SYWMH |
| | HCDR1 | | | |
| 178. | CH3 08-A11 CC - | artificial | aa | KIDPSDDYTNYNQKVKG |
| | HCDR2 | | | |
| 179. | CH3 08-A11 CC - | artificial | aa | WDYNYFDV |
| | HCDR3 | | | |
| 180. | CH3 08-A11 CC - | artificial | aa | RASSSVSYMH |
| | LCDR1 | | | |
| 181. | CH3 08-A11 CC - | artificial | aa | GTSNLVS |
| | LCDR2 | | | |
| 182. | CH3 08-A11 CC - | artificial | aa | QQWSSYPLT |
| | LCDR3 | | | |
| 183. | CH3 08-A11 CC - | artificial | aa | EVQLLESGGGLVQPGGSLRLSCAASGFTFSSY |
| | VH | | | WMHWVRQTPGKCLEWVSKIDPSDDYTNYNQ |
| | | | | KVKGRFTISIDKSKNTLYLQMNSLRAEDTAVY |
| | | | | YCARWDYNYFDVWGQGTTVTVSS |
| 184. | CH3 08-A11 CC - | artificial | aa | EIVMTQSPATLSVSPGERATLTCRASSSVSYMH |
| | VL | | | WYQQKPGQAPRLLIYGTSNLVSGVPARFSGSG |
| | | | | SGTEFTLTISSLQSEDFAVYYCQQWSSYPLTFG |
| | | | | CGTKVEIK |
| 185. | CH3 14-D1 CC - | artificial | Aa | SYWMH |
| | HCDR1 | | | |
| 186. | CH3 14-D1 CC - | artificial | Aa | VIYTSGSYTIYNQKFQG |
| | HCDR2 | | | |
| 187. | CH3 14-D1 CC - | artificial | Aa | SGPGYFDV |
| | HCDR3 | | | |
| 188. | CH3 14-D1 CC - | artificial | Aa | RASGNIHNYLA |
| | LCDR1 | | | |
| 189. | CH3 14-D1 CC - | artificial | Aa | NAKTLAE |
| | LCDR2 | | | |
| 190. | CH3 14-D1 CC - | artificial | Aa | QHFAWTPYT |
| | LCDR3 | | | |
| 191. | CH3 14-D1 CC - | artificial | Aa | QVQLVQSGAEVKKPGASVKVSCKASGYTFTSY |
| | VH | | | WMHWVRQAPGQCLEWMGVIYTSGSYTIYNQ |
| | | | | KFQGRVTMTRDTSTSTAYMELSSLRSEDTAVY |
| | | | | YCARSGPGYFDVWGQGTMVTVSS |
| 192. | CH3 14-D1 CC - | artificial | Aa | DIQLTQSPSFLSASVGDRVTITCRASGNIHNYLA |
| | VL | | | WYQQKPGKAPKLLIYNAKTLAEGVPSRFSGSG |
| | | | | SGTEFTLKISSLQPEDFATYYCQHFAWTPYTFG |
| | | | | CGTKLEIK |
| 193. | CH3 14-D1 CC EI - | artificial | Aa | EIQLTQSPSFLSASVGDRVTITCRASGNIHNYLA |
| | VL | | | WYQQKPGKAPKLLIYNAKTLAEGVPSRFSGSG |
| | | | | SGTEFTLKISSLQPEDFATYYCQHFAWTPYTFG |
| | | | | CGTKLEIK |
| 194. | CH3 15-E11 CC - | artificial | aa | NYWMN |
| | HCDR1 | | | |
| 195. | CH3 15-E11 CC - | artificial | aa | NIAYGVKGTNYNQKFQG |
| | HCDR2 | | | |
| 196. | CH3 15-E11 CC - | artificial | aa | RYFYVMDY |
| | HCDR3 | | | |
| 197. | CH3 15-E11 CC - | artificial | aa | RASQDISNYLN |
| | LCDR1 | | | |
| 198. | CH3 15-E11 CC - | artificial | aa | YTSRLHS |
| | LCDR2 | | | |
| 199. | CH3 15-E11 CC - | artificial | aa | VQYAQFPLT |
| | LCDR3 | | | |
| 200. | CH3 15-E11 CC - | artificial | aa | QVQLVQSGAEVKKPGASVKVSCKASGYTFTN |
| | VH | | | YWMNWVRQAPGQCLEWMGNIAYGVKGTNY |
| | | | | NQKFQGRVTMTVDTSSSTAYMELSRLRSDDTA |
| | | | | VYYCATRYFYVMDYWGQGTLVTVSS |
| 201. | CH3 15-E11 CC - | artificial | aa | DIQMTQSPSSLSASVGDRVTITCRASQDISNYL |
| | VL | | | NWYQQKPGKVPKLLIYYTSRLHSGVPSRFSGS |
| | | | | GSGTDFTLTISSLQPEDVATYYCVQYAQFPLTF |
| | | | | GCGTKVEIK |
| 202. | CH3 15-E11 CC | artificial | Aa | EIQMTQSPSSLSASVGDRVTITCRASQDISNYLN |
| | EI - VL | | | WYQQKPGKVPKLLIYYTSRLHSGVPSRFSGSGS |
| | | | | GTDFTLTISSLQPEDVATYYCVQYAQFPLTFGC |
| | | | | GTKVEIK |
| 203. | CH3 22-A12 CC - | artificial | Aa | SSWMN |
| | HCDR1 | | | |
| 204. | CH3 22-A12 CC - | artificial | Aa | RIYTGTGETKYSGKFQG |
| | HCDR2 | | | |
| 205. | CH3 22-A12 CC - | artificial | Aa | QRDYGALYAMDY |
| | HCDR3 | | | |
| 206. | CH3 22-A12 CC - | artificial | Aa | RASDDIYSYLA |
| | LCDR1 | | | |
| 207. | CH3 22-A12 CC - | artificial | Aa | NAKTLAE |
| | LCDR2 | | | |
| 208. | CH3 22-A12 CC - | artificial | Aa | QNHDRTPFT |
| | LCDR3 | | | |
| 209. | CH3 22-A12 CC - | artificial | Aa | QVQLVQSGAEVVKPGASVKVSCKASGYTFTSS |
| | VH | | | WMNWVRQAPGQCLEWMGRIYTGTGETKYSG |
| | | | | KFQGRVTITRDTSASTAYMELSSLTSEDTAVYY |
| | | | | CARQRDYGALYAMDYWGQGTLVTVSS |
| 210. | CH3 22-A12 CC - | artificial | Aa | DIQLTQSPSFLSASVGDRVTITCRASDDIYSYLA |
| | VL | | | WYQQKPGKAPKLLVYNAKTLAEGVPSRFSGS |
| | | | | GSGTEFTLTISSLQPEDFATYYCQNHDRTPFTFG |
| | | | | CGTKVDIK |
| 211. | CH3 22-A12 CC | artificial | aa | EIQLTQSPSFLSASVGDRVTITCRASDDIYSYLA |
| | EI - VL | | | WYQQKPGKAPKLLVYNAKTLAEGVPSRFSGS |
| | | | | GSGTEFTLTISSLQPEDFATYYCQNHDRTPFTFG |
| | | | | CGTKVDIK |
| 212. | CH3 24-D7 CC - | artificial | aa | NYWMN |
| | HCDR1 | | | |
| 213. | CH3 24-D7 CC - | artificial | aa | NIHSKAHGTNYNQKFQG |
| | HCDR2 | | | |
| 214. | CH3 24-D7 CC - | artificial | aa | RYFYVMDY |
| | HCDR3 | | | |
| 215. | CH3 24-D7 CC - | artificial | aa | RASQDISNYLN |
| | LCDR1 | | | |
| 216. | CH3 24-D7 CC - | artificial | aa | YTSRLHS |
| | LCDR2 | | | |
| 217. | CH3 24-D7 CC - | artificial | aa | VQYAQFPLT |
| | LCDR3 | | | |
| 218. | CH3 24-D7 CC - | artificial | aa | QVQLVQSGAEVKKPGASVKVSCKASGYTFTN |
| | VH | | | YWMNWVRQAPGQCLEWMGNIHSKAHGTNY |
| | | | | NQKFQGRVTMTVDTSSSTAYMELSRLRSDDTA |
| | | | | VYYCATRYFYVMDYWGQGTLVTVSS |
| 219. | CH3 24-D7 CC - | artificial | Aa | DIQMTQSPSSLSASVGDRVTITCRASQDISNYL |
| | VL | | | NWYQQKPGKVPKLLIYYTSRLHSGVPSRFSGS |
| | | | | GSGTDFTLTISSLQPEDVATYYCVQYAQFPLTF |
| | | | | GCGTKVEIK |
| 220. | CH3 24-D7 CC EI - | artificial | Aa | EIQMTQSPSSLSASVGDRVTITCRASQDISNYLN |
| | VL | | | WYQQKPGKVPKLLIYYTSRLHSGVPSRFSGSGS |
| | | | | GTDFTLTISSLQPEDVATYYCVQYAQFPLTFGC |
| | | | | GTKVEIK |
| 221. | CH3 26-E5 CC - | artificial | Aa | SYWMH |
| | HCDR1 | | | |
| 222. | CH3 26-E5 CC - | artificial | Aa | VIRTSTSYTIYNQKFKG |
| | HCDR2 | | | |
| 223. | CH3 26-E5 CC - | artificial | Aa | SGPGYFDV |
| | HCDR3 | | | |
| 224. | CH3 26-E5 CC - | artificial | Aa | RASENIYSYLA |
| | LCDR1 | | | |
| 225. | CH3 26-E5 CC - | artificial | Aa | NAKTLAE |
| | LCDR2 | | | |
| 226. | CH3 26-E5 CC - | artificial | Aa | QHNYGTPYT |
| | LCDR3 | | | |
| 227. | CH3 26-E5 CC - | artificial | Aa | QVQLVQSGAEVKKPGASVKVSCKASGYTFTSY |
| | VH | | | WMHWVRQAPGQCLEWMGVIRTSTSYTIYNQK |
| | | | | FKGRVTMTRDTSTSTVYMELSSLRSEDTAVYY |
| | | | | CARSGPGYFDVWGQGTMVTVSS |
| 228. | CH3 26-E5 CC - | artificial | aa | DIQLTQSPSFLSASVGDRVTITCRASENIYSYLA |
| | VL | | | WYQQKPGKAPKLLIYNAKTLAEGVPSRFSGSG |
| | | | | SGTEFTLTISSLQPEDFATYYCQHNYGTPYTFG |
| | | | | CGTKLEIK |
| 229. | CH3 26-E5 CC EI - | artificial | aa | EIQLTQSPSFLSASVGDRVTITCRASENIYSYLA |
| | VL | | | WYQQKPGKAPKLLIYNAKTLAEGVPSRFSGSG |
| | | | | SGTEFTLTISSLQPEDFATYYCQHNYGTPYTFG |
| | | | | CGTKLEIK |
| 230. | CH3 R164L CC - | artificial | aa | SYWMY |
| | HCDR1 | | | |
| 231. | CH3 R164L CC - | artificial | aa | KIDPSDDYTNYNQKVKG |
| | HCDR2 | | | |
| 232. | CH3 R164L CC - | artificial | aa | WDYTHFDV |
| | HCDR3 | | | |
| 233. | CH3 R164L CC - | artificial | aa | RASSSVSYMH |
| | LCDR1 | | | |
| 234. | CH3 R164L CC - | artificial | aa | GTSNLAS |
| | LCDR2 | | | |
| 235. | CH3 R164L CC - | artificial | aa | QQWSSYPLT |
| | LCDR3 | | | |
| 236. | CH3 R164L CC - | artificial | Aa | EVQLLESGGGLVQPGGSVRLSCAASGFTESSY |
| | VH | | | WMYWVRQAPGKCLEWVSKIDPSDDYTNYNQ |
| | | | | KVKGRFTISIDNSKNTLYLQMNSLRAEDSAVY |
| | | | | YCARWDYTHFDVWGQGTTVTVSS |
| 237. | CH3 R164L CC - | artificial | Aa | EIVMTQSPATLSVSPGERATLSCRASSSVSYMH |
| | VL | | | WYQQKPGQAPRLLIYGTSNLASGVPVRFSGSG |
| | | | | SGTEFTLTISRLQSEDVAVYYCQQWSSYPLTFG |
| | | | | CGTKVEIK |
| 238. | CH3 R170R CC - | artificial | Aa | SYWMH |
| | HCDR1 | | | |
| 239. | CH3 R170R CC - | artificial | Aa | KIDPSDDYTNYNQKVKG |
| | HCDR2 | | | |
| 240. | CH3 R170R CC - | artificial | Aa | WDYSHFDV |
| | HCDR3 | | | |
| 241. | CH3 R170R CC - | artificial | Aa | RASSSVSYMH |
| | LCDR1 | | | |
| 242. | CH3 R170R CC - | artificial | Aa | GTSNLVS |
| | LCDR2 | | | |
| 243. | CH3 R170R CC - | artificial | Aa | QQWSSYPLT |
| | LCDR3 | | | |
| 244. | CH3 R170R CC - | artificial | Aa | EVQLLESGGGLVQPGGSLRLSCAASGFTFSSY |
| | VH | | | WMHWVRQTPGKCLEWVSKIDPSDDYTNYNQ |
| | | | | KVKGRFTISIDKSKNTLYLQMNSLRAEDTAVY |
| | | | | YCARWDYSHFDVWGQGTTVTVSS |
| 245. | CH3 R170R CC - | artificial | aa | EIVMTQSPATLSVSPGERATLTCRASSSVSYMH |
| | VL | | | WYQQKPGQAPRLLIYGTSNLVSGVPARFSGSG |
| | | | | SGTEFTLTISSLQSEDFAVYYCQQWSSYPLTFG |
| | | | | CGTKVEIK |
| 246. | CH3 005-D5 CCx | artificial | aa | EVQLLESGGGLVQPGGSLRLSCAASGFSFSSYPI |
| | I2Ccc(44/100)x | | | NWVRQAPGKCLEWVGVIWTGGGTNYASSVK |
| | (G4)x scFc x (G4) | | | GRFTISRDNSKNTVYLQMNSLRAEDTAVYYCA |
| | x MS 01-G11 CCx | | | KSRGVYDFKGRGAMDYWGQGTLVTVSSGGG |
| | I2Ccc(44/100) - | | | GSGGGGSGGGGSDIVMTQSPDSLAVSLGERATI |
| | Full Sequence | | | NCKSSQSLLYSSNQKNYFAWYQQKPGQPPKLL |
| | | | | IYWASTRESGVPDRFSGSGSGTDFTLTISSLQAE |
| | | | | DVAVYYCQQYYSYPYTFGCGTKLEIKSGGGGS |
| | | | | EVQLVESGGGLVQPGGSLKLSCAASGFTFNKY |
| | | | | AMNWVRQAPGKCLEWVARIRSKYNNYATYY |
| | | | | ADSVKDRFTISRDDSKNTAYLQMNNLKTEDTA |
| | | | | VYYCVRHGNFGNSYISYWAYWGQGTLVTVSS |
| | | | | GGGGSGGGGSGGGGSQTVVTQEPSLTVSPGGT |
| | | | | VTLTCGSSTGAVTSGNYPNWVQQKPGQAPRG |
| | | | | LIGGTKFLAPGTPARFSGSLLGGKAALTLSGVQ |
| | | | | PEDEAEYYCVLWYSNRWVFGCGTKLTVLGGG |
| | | | | GDKTHTCPPCPAPELLGGPSVFLFPPKPKDTLM |
| | | | | ISRTPEVTCVVVDVSHEDPEVKFNWYVDGVEV |
| | | | | HNAKTKPCEEQYGSTYRCVSVLTVLHQDWLN |
| | | | | GKEYKCKVSNKALPAPIEKTISKAKGQPREPQV |
| | | | | YTLPPSREEMTKNQVSLTCLVKGFYPSDIAVE |
| | | | | WESNGQPENNYKTTPPVLDSDGSFFLYSKLTV |
| | | | | DKSRWQQGNVFSCSVMHEALHNHYTQKSLSL |
| | | | | SPGKGGGGSGGGGSGGGGSGGGGSGGGGSGG |
| | | | | GGSDKTHTCPPCPAPELLGGPSVFLFPPKPKDT |
| | | | | LMISRTPEVTCVVVDVSHEDPEVKFNWYVDG |
| | | | | VEVHNAKTKPCEEQYGSTYRCVSVLTVLHQD |
| | | | | WLNGKEYKCKVSNKALPAPIEKTISKAKGQPR |
| | | | | EPQVYTLPPSREEMTKNQVSLTCLVKGFYPSDI |
| | | | | AVEWESNGQPENNYKTTPPVLDSDGSFFLYSK |
| | | | | LTVDKSRWQQGNVFSCSVMHEALHNHYTQKS |
| | | | | LSLSPGKGGGGQVQLVESGGGLVKPGGSLRLS |
| | | | | CAASGFTFSDYYMTWIRQAPGKCLEWLSYISSS |
| | | | | GSTIYYAEAVKGRFTISRDNAKNSLFLQMNSLR |
| | | | | AEDTAVYYCARDRNSHFDYWGQGTLVTVSSG |
| | | | | GGGSGGGGSGGGGSDIMTQSPSSVSASVGDRV |
| | | | | TITCRASQGIRTWLAWYQQKPGKAPKLLIYGA |
| | | | | SGLQSGVPSRFSGSGSGTDFTLTISSLQPEDFAT |
| | | | | YYCQQAESFPRTFGCGTKVEIKSGGGGSEVQL |
| | | | | VESGGGLVQPGGSLKLSCAASGFTFNKYAMN |
| | | | | WVRQAPGKCLEWVARIRSKYNNYATYYADSV |
| | | | | KDRFTISRDDSKNTAYLQMNNLKTEDTAVYYC |
| | | | | VRHGNFGNSYISYWAYWGQGTLVTVSSGGGG |
| | | | | SGGGGSGGGGSQTVVTQEPSLTVSPGGTVTLT |
| | | | | CGSSTGAVTSGNYPNWVQQKPGQAPRGLIGGT |
| | | | | KFLAPGTPARFSGSLLGGKAALTLSGVQPEDEA |
| | | | | EYYCVLWYSNRWVFGCGTKLTVL |
| 247. | CH3 08-A11 CC x | artificial | aa | EVQLLESGGGLVQPGGSLRLSCAASGFTFSSY |
| | I2Ccc(44/100)x | | | WMHWVRQTPGKCLEWVSKIDPSDDYTNYNQ |
| | (G4S)3x scFcx | | | KVKGRFTISIDKSKNTLYLQMNSLRAEDTAVY |
| | (G4S)3x MS R4L | | | YCARWDYNYFDVWGQGTTVTVSSGGGGSGG |
| | CCx | | | GGSGGGGSEIVMTQSPATLSVSPGERATLTCRA |
| | I2Ccc(44/100) - | | | SSSVSYMHWYQQKPGQAPRLLIYGTSNLVSGV |
| | Full Sequence | | | PARFSGSGSGTEFTLTISSLQSEDFAVYYCQQW |
| | | | | SSYPLTFGCGTKVEIKSGGGGSEVQLVESGGGL |
| | | | | VQPGGSLKLSCAASGFTFNKYAMNWVRQAPG |
| | | | | KCLEWVARIRSKYNNYATYYADSVKDRFTISR |
| | | | | DDSKNTAYLQMNNLKTEDTAVYYCVRHGNFG |
| | | | | NSYISYWAYWGQGTLVTVSSGGGGSGGGGSG |
| | | | | GGGSQTVVTQEPSLTVSPGGTVTLTCGSSTGA |
| | | | | VTSGNYPNWVQQKPGQAPRGLIGGTKFLAPGT |
| | | | | PARFSGSLLGGKAALTLSGVQPEDEAEYYCVL |
| | | | | WYSNRWVFGCGTKLTVLGGGGSGGGGSGGG |
| | | | | GSDKTHTCPPCPAPELLGGPSVFLFPPKPKDTL |
| | | | | MISRTPEVTCVVVDVSHEDPEVKFNWYVDGV |
| | | | | EVHNAKTKPCEEQYGSTYRCVSVLTVLHQDW |
| | | | | LNGKEYKCKVSNKALPAPIEKTISKAKGQPREP |
| | | | | QVYTLPPSREEMTKNQVSLTCLVKGFYPSDIAV |
| | | | | EWESNGQPENNYKTTPPVLDSDGSFFLYSKLT |
| | | | | VDKSRWQQGNVFSCSVMHEALHNHYTQKSLS |
| | | | | LSPGKGGGGSGGGGSGGGGSGGGGSGGGGSG |
| | | | | GGGSDKTHTCPPCPAPELLGGPSVFLFPPKPKD |
| | | | | TLMISRTPEVTCVVVDVSHEDPEVKFNWYVDG |
| | | | | VEVHNAKTKPCEEQYGSTYRCVSVLTVLHQD |
| | | | | WLNGKEYKCKVSNKALPAPIEKTISKAKGQPR |
| | | | | EPQVYTLPPSREEMTKNQVSLTCLVKGFYPSDI |
| | | | | AVEWESNGQPENNYKTTPPVLDSDGSFFLYSK |
| | | | | LTVDKSRWQQGNVFSCSVMHEALHNHYTQKS |
| | | | | LSLSPGKGGGGSGGGGSGGGGSQVQLVQSGA |
| | | | | EVKKPGASVKVSCKASGYTFTGYYIHWVRQA |
| | | | | PGQCLEWMGWINPNSGGTNYAQKFQGRVTMT |
| | | | | RDTSISTAYMELSRLRSDDTAVYYCARVEAVA |
| | | | | GREYYYFSGMDVWGQGTTVTVSSGGGGSGGG |
| | | | | GSGGGGSSYELTQPPSVSVSPGQTASITCSGEK |
| | | | | LGDKYVYWYQQKPGQSPVLVIYQSTKRPSGVP |
| | | | | ERFSGSNSGNTATLTISGTQAMDEADYYCQAY |
| | | | | HASTAVFGCGTKLTVLSGGGGSEVQLVESGGG |
| | | | | LVQPGGSLKLSCAASGFTFNKYAMNWVRQAP |
| | | | | GKCLEWVARIRSKYNNYATYYADSVKDRFTIS |
| | | | | RDDSKNTAYLQMNNLKTEDTAVYYCVRHGNF |
| | | | | GNSYISYWAYWGQGTLVTVSSGGGGSGGGGS |
| | | | | GGGGSQTVVTQEPSLTVSPGGTVTLTCGSSTG |
| | | | | AVTSGNYPNWVQQKPGQAPRGLIGGTKFLAPG |
| | | | | TPARFSGSLLGGKAALTLSGVQPEDEAEYYCV |
| | | | | LWYSNRWVFGCGTKLTVL |
| 248. | CH3 08-A11 CCx | artificial | aa | EVQLLESGGGLVQPGGSLRLSCAASGFTFSSY |
| | 6H10.09x | | | WMHWVRQTPGKCLEWVSKIDPSDDYTNYNQ |
| | (G4S)3x scFcx | | | KVKGRFTISIDKSKNTLYLQMNSLRAEDTAVY |
| | (G4S)3x MS R4L | | | YCARWDYNYFDVWGQGTTVTVSSGGGGSGG |
| | CCx 6H10.09 - | | | GGSGGGGSEIVMTQSPATLSVSPGERATLTCRA |
| | Full Sequence | | | SSSVSYMHWYQQKPGQAPRLLIYGTSNLVSGV |
| | | | | PARFSGSGSGTEFTLTISSLQSEDFAVYYCQQW |
| | | | | SSYPLTFGCGTKVEIKSGGGGSEVQLVESGGGL |
| | | | | VQPGGSLKLSCAASGFTFNKYAMNWVRQAPG |
| | | | | KGMEWVARIRSKYNNYATYYADAVKDRFTIS |
| | | | | RDDSKNTLYLQMNNLKTEDTAVYYCVRAGNF |
| | | | | GSSYISYFAYWGQGTLVTVSSGGGGSGGGGSG |
| | | | | GGGSQTVVTQEPSLTVSPGGTVTITCGSSTGAV |
| | | | | TSGNYPNWIQKKPGQAPRGLIGGTKFLAPGTP |
| | | | | ARFSGSLEGGKAALTLSGVQPEDEAEYYCVLY |
| | | | | YSNRWVFGSGTKLTVLGGGGSGGGGSGGGGS |
| | | | | DKTHTCPPCPAPELLGGPSVFLFPPKPKDTLMIS |
| | | | | RTPEVTCVVVDVSHEDPEVKFNWYVDGVEVH |
| | | | | NAKTKPCEEQYGSTYRCVSVLTVLHQDWLNG |
| | | | | KEYKCKVSNKALPAPIEKTISKAKGQPREPQVY |
| | | | | TLPPSREEMTKNQVSLTCLVKGFYPSDIAVEWE |
| | | | | SNGQPENNYKTTPPVLDSDGSFFLYSKLTVDKS |
| | | | | RWQQGNVFSCSVMHEALHNHYTQKSLSLSPG |
| | | | | KGGGGSGGGGSGGGGSGGGGSGGGGSGGGGS |
| | | | | DKTHTCPPCPAPELLGGPSVFLFPPKPKDTLMIS |
| | | | | RTPEVTCVVVDVSHEDPEVKFNWYVDGVEVH |
| | | | | NAKTKPCEEQYGSTYRCVSVLTVLHQDWLNG |
| | | | | KEYKCKVSNKALPAPIEKTISKAKGQPREPQVY |
| | | | | TLPPSREEMTKNQVSLTCLVKGFYPSDIAVEWE |
| | | | | SNGQPENNYKTTPPVLDSDGSFFLYSKLTVDKS |
| | | | | RWQQGNVFSCSVMHEALHNHYTQKSLSLSPG |
| | | | | KGGGGSGGGGSGGGGSQVQLVQSGAEVKKPG |
| | | | | ASVKVSCKASGYTFTGYYIHWVRQAPGQCLE |
| | | | | WMGWINPNSGGTNYAQKFQGRVTMTRDTSIS |
| | | | | TAYMELSRLRSDDTAVYYCARVEAVAGREYY |
| | | | | YFSGMDVWGQGTTVTVSSGGGGSGGGGSGGG |
| | | | | GSSYELTQPPSVSVSPGQTASITCSGEKLGDKY |
| | | | | VYWYQQKPGQSPVLVIYQSTKRPSGVPERFSG |
| | | | | SNSGNTATLTISGTQAMDEADYYCQAYHASTA |
| | | | | VFGCGTKLTVLSGGGGSEVQLVESGGGLVQPG |
| | | | | GSLKLSCAASGFTFNKYAMNWVRQAPGKGME |
| | | | | WVARIRSKYNNYATYYADAVKDRFTISRDDSK |
| | | | | NTLYLQMNNLKTEDTAVYYCVRAGNFGSSYIS |
| | | | | YFAYWGQGTLVTVSSGGGGSGGGGSGGGGSQ |
| | | | | TVVTQEPSLTVSPGGTVTITCGSSTGAVTSGNY |
| | | | | PNWIQKKPGQAPRGLIGGTKFLAPGTPARFSGS |
| | | | | LEGGKAALTLSGVQPEDEAEYYCVLYYSNRW |
| | | | | VFGSGTKLTVL |
| 249. | CH3 08-A11 CCx | artificial | aa | EVQLLESGGGLVQPGGSLRLSCAASGFTFSSY |
| | I2Ccc(44/100)x | | | WMHWVRQTPGKCLEWVSKIDPSDDYTNYNQ |
| | (G4)x scFc x (G4) | | | KVKGRFTISIDKSKNTLYLQMNSLRAEDTAVY |
| | x MS R4L CCx | | | YCARWDYNYFDVWGQGTTVTVSSGGGGSGG |
| | I2Ccc(44/100) - | | | GGSGGGGSEIVMTQSPATLSVSPGERATLTCRA |
| | Full Sequence | | | SSSVSYMHWYQQKPGQAPRLLIYGTSNLVSGV |
| | | | | PARFSGSGSGTEFTLTISSLQSEDFAVYYCQQW |
| | | | | SSYPLTFGCGTKVEIKSGGGGSEVQLVESGGGL |
| | | | | VQPGGSLKLSCAASGFTFNKYAMNWVRQAPG |
| | | | | KCLEWVARIRSKYNNYATYYADSVKDRFTISR |
| | | | | DDSKNTAYLQMNNLKTEDTAVYYCVRHGNFG |
| | | | | NSYISYWAYWGQGTLVTVSSGGGGSGGGGSG |
| | | | | GGGSQTVVTQEPSLTVSPGGTVTLTCGSSTGA |
| | | | | VTSGNYPNWVQQKPGQAPRGLIGGTKFLAPGT |
| | | | | PARFSGSLLGGKAALTLSGVQPEDEAEYYCVL |
| | | | | WYSNRWVFGCGTKLTVLGGGGDKTHTCPPCP |
| | | | | APELLGGPSVFLFPPKPKDTLMISRTPEVTCVV |
| | | | | VDVSHEDPEVKFNWYVDGVEVHNAKTKPCEE |
| | | | | QYGSTYRCVSVLTVLHQDWLNGKEYKCKVSN |
| | | | | KALPAPIEKTISKAKGQPREPQVYTLPPSREEM |
| | | | | TKNQVSLTCLVKGFYPSDIAVEWESNGQPENN |
| | | | | YKTTPPVLDSDGSFFLYSKLTVDKSRWQQGNV |
| | | | | FSCSVMHEALHNHYTQKSLSLSPGKGGGGSGG |
| | | | | GGSGGGGSGGGGSGGGGSGGGGSDKTHTCPP |
| | | | | CPAPELLGGPSVFLFPPKPKDTLMISRTPEVTCV |
| | | | | VVDVSHEDPEVKFNWYVDGVEVHNAKTKPCE |
| | | | | EQYGSTYRCVSVLTVLHQDWLNGKEYKCKVS |
| | | | | NKALPAPIEKTISKAKGQPREPQVYTLPPSREE |
| | | | | MTKNQVSLTCLVKGFYPSDIAVEWESNGQPEN |
| | | | | NYKTTPPVLDSDGSFFLYSKLTVDKSRWQQGN |
| | | | | VFSCSVMHEALHNHYTQKSLSLSPGKGGGGQ |
| | | | | VQLVQSGAEVKKPGASVKVSCKASGYTFTGY |
| | | | | YIHWVRQAPGQCLEWMGWINPNSGGTNYAQ |
| | | | | KFQGRVTMTRDTSISTAYMELSRLRSDDTAVY |
| | | | | YCARVEAVAGREYYYFSGMDVWGQGTTVTV |
| | | | | SSGGGGSGGGGSGGGGSSYELTQPPSVSVSPG |
| | | | | QTASITCSGEKLGDKYVYWYQQKPGQSPVLVI |
| | | | | YQSTKRPSGVPERFSGSNSGNTATLTISGTQAM |
| | | | | DEADYYCQAYHASTAVFGCGTKLTVLSGGGG |
| | | | | SEVQLVESGGGLVQPGGSLKLSCAASGFTFNK |
| | | | | YAMNWVRQAPGKCLEWVARIRSKYNNYATY |
| | | | | YADSVKDRFTISRDDSKNTAYLQMNNLKTEDT |
| | | | | AVYYCVRHGNFGNSYISYWAYWGQGTLVTVS |
| | | | | SGGGGSGGGGSGGGGSQTVVTQEPSLTVSPGG |
| | | | | TVTLTCGSSTGAVTSGNYPNWVQQKPGQAPR |
| | | | | GLIGGTKFLAPGTPARFSGSLLGGKAALTLSGV |
| | | | | QPEDEAEYYCVLWYSNRWVFGCGTKLTVL |
| 250. | CH3 15-E11 CC x | artificial | aa | QVQLVQSGAEVKKPGASVKVSCKASGYTFTN |
| | I2L x (G4Q)3x | | | YWMNWVRQAPGQCLEWMGNIAYGVKGTNY |
| | scFcmod x | | | NQKFQGRVTMTVDTSSSTAYMELSRLRSDDTA |
| | (G4Q)3 x MS 15- | | | VYYCATRYFYVMDYWGQGTLVTVSSGGGGQ |
| | B12 CC x I2L - | | | GGGGQGGGGQDIQMTQSPSSLSASVGDRVTIT |
| | Full Sequence | | | CRASQDISNYLNWYQQKPGKVPKLLIYYTSRL |
| | | | | HSGVPSRFSGSGSGTDFTLTISSLQPEDVATYYC |
| | | | | VQYAQFPLTFGCGTKVEIKSGGGGQEVQLVES |
| | | | | GGGLVQPGGSLKLSCAASGFTFNKYAMNWVR |
| | | | | QAPGKGMEWVARIRSKYNNYATYYADAVKD |
| | | | | RFTISRDDSKNTLYLQMNNLKTEDTAVYYCVR |
| | | | | AGNFGSSYISYFAYWGQGTLVTVSSGGGGQGG |
| | | | | GGQGGGGQQTVVTQEPSLTVSPGGTVTITCGS |
| | | | | STGAVTSGNYPNWIQKKPGQAPRGLIGGTKFL |
| | | | | APGTPARFSGSLEGGKAALTLSGVQPEDEAEY |
| | | | | YCVLYYSNRWVFGSGTKLTVLGGGGQGGGGQ |
| | | | | GGGGQCPPCPAPELLGGPSVFLFPPKPKDTLMI |
| | | | | SRTPEVTCVVVDVSHEEPEVKFNWYVDGVEV |
| | | | | HNAKTKPCEEQYGSTYRCVSVLTVLHQDWLN |
| | | | | GKEYKCKVSNKALPAPIEKTISKAKGQPREPQV |
| | | | | YTLPPSREEMTKNQVSLTCLVKGFYPSDIAVE |
| | | | | WESNGQPENNYKTTPPVLDSDGSFFLYSKLTV |
| | | | | DKSRWQQGNVFSCSVMHEALHNHYTQKSLSL |
| | | | | SPGKGGGGQGGGGQGGGGQGGGGQGGGGQG |
| | | | | GGGQCPPCPAPELLGGPSVFLFPPKPKDTLMIS |
| | | | | RTPEVTCVVVDVSHEEPEVKFNWYVDGVEVH |
| | | | | NAKTKPCEEQYGSTYRCVSVLTVLHQDWLNG |
| | | | | KEYKCKVSNKALPAPIEKTISKAKGQPREPQVY |
| | | | | TLPPSREEMTKNQVSLTCLVKGFYPSDIAVEWE |
| | | | | SNGQPENNYKTTPPVLDSDGSFFLYSKLTVDKS |
| | | | | RWQQGNVFSCSVMHEALHNHYTQKSLSLSPG |
| | | | | KGGGGQGGGGQGGGGQQVQLQESGPGLVKPS |
| | | | | ETLSLTCTVSGGSISSSSYFWGWIRQPPGKCLE |
| | | | | WIGNIYYSGSSNYNPSLKSRVTISVDTSKNQFSL |
| | | | | KLSSVTAADTAVYYCARLPRGDRDAFDIWGQ |
| | | | | GTMVTVSSGGGGQGGGGQGGGGQDIVMTQSP |
| | | | | SSLSASVGDRVTITCRASQGISNYLAWYQQKP |
| | | | | GKVPKLLIYAASTLQSGVPSRFSGSGSGTDFTL |
| | | | | TISSLQPEDFATYYCQQSYSTPFTFGCGTKVEIK |
| | | | | SGGGGQEVQLVESGGGLVQPGGSLKLSCAASG |
| | | | | FTFNKYAMNWVRQAPGKGMEWVARIRSKYN |
| | | | | NYATYYADAVKDRFTISRDDSKNTLYLQMNN |
| | | | | LKTEDTAVYYCVRAGNFGSSYISYFAYWGQGT |
| | | | | LVTVSSGGGGQGGGGQGGGGQQTVVTQEPSL |
| | | | | TVSPGGTVTITCGSSTGAVTSGNYPNWIQKKPG |
| | | | | QAPRGLIGGTKFLAPGTPARFSGSLEGGKAALT |
| | | | | LSGVQPEDEAEYYCVLYYSNRWVFGSGTKLT |
| | | | | VL |
| 251. | CH3 15-E11 CC x | artificial | aa | QVQLVQSGAEVKKPGASVKVSCKASGYTFTN |
| | I2L x G4 x scFc x | | | YWMNWVRQAPGQCLEWMGNIAYGVKGTNY |
| | G4 x MS 15-B12 | | | NQKFQGRVTMTVDTSSSTAYMELSRLRSDDTA |
| | CC x I2L_GQ - | | | VYYCATRYFYVMDYWGQGTLVTVSSGGGGQ |
| | Full Sequence | | | GGGGQGGGGQDIQMTQSPSSLSASVGDRVTIT |
| | | | | CRASQDISNYLNWYQQKPGKVPKLLIYYTSRL |
| | | | | HSGVPSRFSGSGSGTDFTLTISSLQPEDVATYYC |
| | | | | VQYAQFPLTFGCGTKVEIKSGGGGQEVQLVES |
| | | | | GGGLVQPGGSLKLSCAASGFTFNKYAMNWVR |
| | | | | QAPGKGMEWVARIRSKYNNYATYYADAVKD |
| | | | | RFTISRDDSKNTLYLQMNNLKTEDTAVYYCVR |
| | | | | AGNFGSSYISYFAYWGQGTLVTVSSGGGGQGG |
| | | | | GGQGGGGQQTVVTQEPSLTVSPGGTVTITCGS |
| | | | | STGAVTSGNYPNWIQKKPGQAPRGLIGGTKFL |
| | | | | APGTPARFSGSLEGGKAALTLSGVQPEDEAEY |
| | | | | YCVLYYSNRWVFGSGTKLTVLGGGGCPPCPAP |
| | | | | ELLGGPSVFLFPPKPKDTLMISRTPEVTCVVVD |
| | | | | VSHEEPEVKFNWYVDGVEVHNAKTKPCEEQY |
| | | | | GSTYRCVSVLTVLHQDWLNGKEYKCKVSNKA |
| | | | | LPAPIEKTISKAKGQPREPQVYTLPPSREEMTK |
| | | | | NQVSLTCLVKGFYPSDIAVEWESNGQPENNYK |
| | | | | TTPPVLDSDGSFFLYSKLTVDKSRWQQGNVFS |
| | | | | CSVMHEALHNHYTQKSLSLSPGKGGGGQGGG |
| | | | | GQGGGGQGGGGQGGGGQGGGGQCPPCPAPEL |
| | | | | LGGPSVFLFPPKPKDTLMISRTPEVTCVVVDVS |
| | | | | HEEPEVKFNWYVDGVEVHNAKTKPCEEQYGS |
| | | | | TYRCVSVLTVLHQDWLNGKEYKCKVSNKALP |
| | | | | APIEKTISKAKGQPREPQVYTLPPSREEMTKNQ |
| | | | | VSLTCLVKGFYPSDIAVEWESNGQPENNYKTT |
| | | | | PPVLDSDGSFFLYSKLTVDKSRWQQGNVFSCS |
| | | | | VMHEALHNHYTQKSLSLSPGKGGGGQVQLQE |
| | | | | SGPGLVKPSETLSLTCTVSGGSISSSSYFWGWIR |
| | | | | QPPGKCLEWIGNIYYSGSSNYNPSLKSRVTISV |
| | | | | DTSKNQFSLKLSSVTAADTAVYYCARLPRGDR |
| | | | | DAFDIWGQGTMVTVSSGGGGQGGGGQGGGG |
| | | | | QDIVMTQSPSSLSASVGDRVTITCRASQGISNY |
| | | | | LAWYQQKPGKVPKLLIYAASTLQSGVPSRFSG |
| | | | | SGSGTDFTLTISSLQPEDFATYYCQQSYSTPFTF |
| | | | | GCGTKVEIKSGGGGQEVQLVESGGGLVQPGGS |
| | | | | LKLSCAASGFTFNKYAMNWVRQAPGKGMEW |
| | | | | VARIRSKYNNYATYYADAVKDRFTISRDDSKN |
| | | | | TLYLQMNNLKTEDTAVYYCVRAGNFGSSYISY |
| | | | | FAYWGQGTLVTVSSGGGGQGGGGQGGGGQQ |
| | | | | TVVTQEPSLTVSPGGTVTITCGSSTGAVTSGNY |
| | | | | PNWIQKKPGQAPRGLIGGTKFLAPGTPARFSGS |
| | | | | LEGGKAALTLSGVQPEDEAEYYCVLYYSNRW |
| | | | | VFGSGTKLTVL |
| 252. | CH3 15-E11 CC x | artificial | aa | QVQLVQSGAEVKKPGASVKVSCKASGYTFTN |
| | I2L x G4 x scFc x | | | YWMNWVRQAPGQCLEWMGNIAYGVKGTNY |
| | G4 x MS 15-B12 | | | NQKFQGRVTMTVDTSSSTAYMELSRLRSDDTA |
| | CC x I2L - Full | | | VYYCATRYFYVMDYWGQGTLVTVSSGGGGS |
| | Sequence | | | GGGGSGGGGSDIQMTQSPSSLSASVGDRVTITC |
| | | | | RASQDISNYLNWYQQKPGKVPKLLIYYTSRLH |
| | | | | SGVPSRFSGSGSGTDFTLTISSLQPEDVATYYCV |
| | | | | QYAQFPLTFGCGTKVEIKSGGGGSEVQLVESG |
| | | | | GGLVQPGGSLKLSCAASGFTFNKYAMNWVRQ |
| | | | | APGKGMEWVARIRSKYNNYATYYADAVKDR |
| | | | | FTISRDDSKNTLYLQMNNLKTEDTAVYYCVRA |
| | | | | GNFGSSYISYFAYWGQGTLVTVSSGGGGSGGG |
| | | | | GSGGGGSQTVVTQEPSLTVSPGGTVTITCGSST |
| | | | | GAVTSGNYPNWIQKKPGQAPRGLIGGTKFLAP |
| | | | | GTPARFSGSLEGGKAALTLSGVQPEDEAEYYC |
| | | | | VLYYSNRWVFGSGTKLTVLGGGGDKTHTCPP |
| | | | | CPAPELLGGPSVFLFPPKPKDTLMISRTPEVTCV |
| | | | | VVDVSHEDPEVKFNWYVDGVEVHNAKTKPCE |
| | | | | EQYGSTYRCVSVLTVLHQDWLNGKEYKCKVS |
| | | | | NKALPAPIEKTISKAKGQPREPQVYTLPPSREE |
| | | | | MTKNQVSLTCLVKGFYPSDIAVEWESNGQPEN |
| | | | | NYKTTPPVLDSDGSFFLYSKLTVDKSRWQQGN |
| | | | | VFSCSVMHEALHNHYTQKSLSLSPGKGGGGSG |
| | | | | GGGSGGGGSGGGGSGGGGSGGGGSDKTHTCP |
| | | | | PCPAPELLGGPSVFLFPPKPKDTLMISRTPEVTC |
| | | | | VVVDVSHEDPEVKFNWYVDGVEVHNAKTKPC |
| | | | | EEQYGSTYRCVSVLTVLHQDWLNGKEYKCKV |
| | | | | SNKALPAPIEKTISKAKGQPREPQVYTLPPSREE |
| | | | | MTKNQVSLTCLVKGFYPSDIAVEWESNGQPEN |
| | | | | NYKTTPPVLDSDGSFFLYSKLTVDKSRWQQGN |
| | | | | VFSCSVMHEALHNHYTQKSLSLSPGKGGGGQ |
| | | | | VQLQESGPGLVKPSETLSLTCTVSGGSISSSSYF |
| | | | | WGWIRQPPGKCLEWIGNIYYSGSSNYNPSLKS |
| | | | | RVTISVDTSKNQFSLKLSSVTAADTAVYYCAR |
| | | | | LPRGDRDAFDIWGQGTMVTVSSGGGGSGGGG |
| | | | | SGGGGSDIVMTQSPSSLSASVGDRVTITCRASQ |
| | | | | GISNYLAWYQQKPGKVPKLLIYAASTLQSGVP |
| | | | | SRFSGSGSGTDFTLTISSLQPEDFATYYCQQSYS |
| | | | | TPFTFGCGTKVEIKSGGGGSEVQLVESGGGLV |
| | | | | QPGGSLKLSCAASGFTFNKYAMNWVRQAPGK |
| | | | | GMEWVARIRSKYNNYATYYADAVKDRFTISR |
| | | | | DDSKNTLYLQMNNLKTEDTAVYYCVRAGNFG |
| | | | | SSYISYFAYWGQGTLVTVSSGGGGSGGGGSGG |
| | | | | GGSQTVVTQEPSLTVSPGGTVTITCGSSTGAVT |
| | | | | SGNYPNWIQKKPGQAPRGLIGGTKFLAPGTPA |
| | | | | RFSGSLEGGKAALTLSGVQPEDEAEYYCVLYY |
| | | | | SNRWVFGSGTKLTVL |
| 253. | CH3 15-E11 CC x | artificial | Aa | QVQLVQSGAEVKKPGASVKVSCKASGYTFTN |
| | I2L x G4S3 x scFc | | | YWMNWVRQAPGQCLEWMGNIAYGVKGTNY |
| | x G4S3 x MS 15- | | | NQKFQGRVTMTVDTSSSTAYMELSRLRSDDTA |
| | B12 CC x I2L - | | | VYYCATRYFYVMDYWGQGTLVTVSSGGGGS |
| | Full Sequence | | | GGGGSGGGGSDIQMTQSPSSLSASVGDRVTITC |
| | | | | RASQDISNYLNWYQQKPGKVPKLLIYYTSRLH |
| | | | | SGVPSRFSGSGSGTDFTLTISSLQPEDVATYYCV |
| | | | | QYAQFPLTFGCGTKVEIKSGGGGSEVQLVESG |
| | | | | GGLVQPGGSLKLSCAASGFTFNKYAMNWVRQ |
| | | | | APGKGMEWVARIRSKYNNYATYYADAVKDR |
| | | | | FTISRDDSKNTLYLQMNNLKTEDTAVYYCVRA |
| | | | | GNFGSSYISYFAYWGQGTLVTVSSGGGGSGGG |
| | | | | GSGGGGSQTVVTQEPSLTVSPGGTVTITCGSST |
| | | | | GAVTSGNYPNWIQKKPGQAPRGLIGGTKFLAP |
| | | | | GTPARFSGSLEGGKAALTLSGVQPEDEAEYYC |
| | | | | VLYYSNRWVFGSGTKLTVLGGGGSGGGGSGG |
| | | | | GGSDKTHTCPPCPAPELLGGPSVFLFPPKPKDT |
| | | | | LMISRTPEVTCVVVDVSHEDPEVKFNWYVDG |
| | | | | VEVHNAKTKPCEEQYGSTYRCVSVLTVLHQD |
| | | | | WLNGKEYKCKVSNKALPAPIEKTISKAKGQPR |
| | | | | EPQVYTLPPSREEMTKNQVSLTCLVKGFYPSDI |
| | | | | AVEWESNGQPENNYKTTPPVLDSDGSFFLYSK |
| | | | | LTVDKSRWQQGNVFSCSVMHEALHNHYTQKS |
| | | | | LSLSPGKGGGGSGGGGSGGGGSGGGGSGGGG |
| | | | | SGGGGSDKTHTCPPCPAPELLGGPSVFLFPPKP |
| | | | | KDTLMISRTPEVTCVVVDVSHEDPEVKFNWYV |
| | | | | DGVEVHNAKTKPCEEQYGSTYRCVSVLTVLH |
| | | | | QDWLNGKEYKCKVSNKALPAPIEKTISKAKGQ |
| | | | | PREPQVYTLPPSREEMTKNQVSLTCLVKGFYPS |
| | | | | DIAVEWESNGQPENNYKTTPPVLDSDGSFFLYS |
| | | | | KLTVDKSRWQQGNVFSCSVMHEALHNHYTQK |
| | | | | SLSLSPGKGGGGSGGGGSGGGGSQVQLQESGP |
| | | | | GLVKPSETLSLTCTVSGGSISSSSYFWGWIRQPP |
| | | | | GKCLEWIGNIYYSGSSNYNPSLKSRVTISVDTS |
| | | | | KNQFSLKLSSVTAADTAVYYCARLPRGDRDAF |
| | | | | DIWGQGTMVTVSSGGGGSGGGGSGGGGSDIV |
| | | | | MTQSPSSLSASVGDRVTITCRASQGISNYLAWY |
| | | | | QQKPGKVPKLLIYAASTLQSGVPSRFSGSGSGT |
| | | | | DFTLTISSLQPEDFATYYCQQSYSTPFTFGCGTK |
| | | | | VEIKSGGGGSEVQLVESGGGLVQPGGSLKLSC |
| | | | | AASGFTFNKYAMNWVRQAPGKGMEWVARIR |
| | | | | SKYNNYATYYADAVKDRFTISRDDSKNTLYLQ |
| | | | | MNNLKTEDTAVYYCVRAGNFGSSYISYFAYW |
| | | | | GQGTLVTVSSGGGGSGGGGSGGGGSQTVVTQ |
| | | | | EPSLTVSPGGTVTITCGSSTGAVTSGNYPNWIQ |
| | | | | KKPGQAPRGLIGGTKFLAPGTPARFSGSLEGGK |
| | | | | AALTLSGVQPEDEAEYYCVLYYSNRWVFGSG |
| | | | | TKLTVL |
| 254. | CH3 15-E11 CC x | artificial | Aa | QVQLVQSGAEVKKPGASVKVSCKASGYTFTN |
| | I2M2 x G4 x scfc | | | YWMNWVRQAPGQCLEWMGNIAYGVKGTNY |
| | x G4 x MS 15- | | | NQKFQGRVTMTVDTSSSTAYMELSRLRSDDTA |
| | B12 CC x I2M2 - | | | VYYCATRYFYVMDYWGQGTLVTVSSGGGGS |
| | Full Sequence | | | GGGGSGGGGSDIQMTQSPSSLSASVGDRVTITC |
| | | | | RASQDISNYLNWYQQKPGKVPKLLIYYTSRLH |
| | | | | SGVPSRFSGSGSGTDFTLTISSLQPEDVATYYCV |
| | | | | QYAQFPLTFGCGTKVEIKSGGGGSEVQLVESG |
| | | | | GGLVQPGGSLKLSCAASGFTFNKYAINWVREA |
| | | | | PGKGLEWVARIRSKYNNYATYYADAVKDRFTI |
| | | | | SRDDSKNTAYLQMNNLKTEDTAVYYCVRNAN |
| | | | | FGTSYISYFAYWGQGTLVTVSSGGGGSGGGGS |
| | | | | GGGGSQTVVTQEPSLTVSPGGTVTLTCGSSTG |
| | | | | AVTSGNYPNWVQKKPGQAPRGLIGGTKFLAPG |
| | | | | TPARFSGSLLGGKAALTLSGVQPEDEAEYYCV |
| | | | | LWYSNRWVFGSGTKLTVLGGGGDKTHTCPPC |
| | | | | PAPELLGGPSVFLFPPKPKDTLMISRTPEVTCVV |
| | | | | VDVSHEDPEVKFNWYVDGVEVHNAKTKPCEE |
| | | | | QYGSTYRCVSVLTVLHQDWLNGKEYKCKVSN |
| | | | | KALPAPIEKTISKAKGQPREPQVYTLPPSREEM |
| | | | | TKNQVSLTCLVKGFYPSDIAVEWESNGQPENN |
| | | | | YKTTPPVLDSDGSFFLYSKLTVDKSRWQQGNV |
| | | | | FSCSVMHEALHNHYTQKSLSLSPGKGGGGSGG |
| | | | | GGSGGGGSGGGGSGGGGSGGGGSDKTHTCPP |
| | | | | CPAPELLGGPSVFLFPPKPKDTLMISRTPEVTCV |
| | | | | VVDVSHEDPEVKFNWYVDGVEVHNAKTKPCE |
| | | | | EQYGSTYRCVSVLTVLHQDWLNGKEYKCKVS |
| | | | | NKALPAPIEKTISKAKGQPREPQVYTLPPSREE |
| | | | | MTKNQVSLTCLVKGFYPSDIAVEWESNGQPEN |
| | | | | NYKTTPPVLDSDGSFFLYSKLTVDKSRWQQGN |
| | | | | VFSCSVMHEALHNHYTQKSLSLSPGKGGGGQ |
| | | | | VQLQESGPGLVKPSETLSLTCTVSGGSISSSSYF |
| | | | | WGWIRQPPGKCLEWIGNIYYSGSSNYNPSLKS |
| | | | | RVTISVDTSKNQFSLKLSSVTAADTAVYYCAR |
| | | | | LPRGDRDAFDIWGQGTMVTVSSGGGGSGGGG |
| | | | | SGGGGSDIVMTQSPSSLSASVGDRVTITCRASQ |
| | | | | GISNYLAWYQQKPGKVPKLLIYAASTLQSGVP |
| | | | | SRFSGSGSGTDFTLTISSLQPEDFATYYCQQSYS |
| | | | | TPFTFGCGTKVEIKSGGGGSEVQLVESGGGLV |
| | | | | QPGGSLKLSCAASGFTFNKYAINWVREAPGKG |
| | | | | LEWVARIRSKYNNYATYYADAVKDRFTISRDD |
| | | | | SKNTAYLQMNNLKTEDTAVYYCVRNANFGTS |
| | | | | YISYFAYWGQGTLVTVSSGGGGSGGGGSGGG |
| | | | | GSQTVVTQEPSLTVSPGGTVTLTCGSSTGAVTS |
| | | | | GNYPNWVQKKPGQAPRGLIGGTKFLAPGTPAR |
| | | | | FSGSLLGGKAALTLSGVQPEDEAEYYCVLWYS |
| | | | | NRWVFGSGTKLTVL |
| 255. | CH3 15-E11 CC x | artificial | Aa | QVQLVQSGAEVKKPGASVKVSCKASGYTFTN |
| | I2M2 x (G4Q)3x | | | YWMNWVRQAPGQCLEWMGNIAYGVKGTNY |
| | scFcmod x | | | NQKFQGRVTMTVDTSSSTAYMELSRLRSDDTA |
| | (G4Q)3 x MS 15- | | | VYYCATRYFYVMDYWGQGTLVTVSSGGGGQ |
| | B12 CC x I2M2 - | | | GGGGQGGGGQDIQMTQSPSSLSASVGDRVTIT |
| | Full Sequence | | | CRASQDISNYLNWYQQKPGKVPKLLIYYTSRL |
| | | | | HSGVPSRFSGSGSGTDFTLTISSLQPEDVATYYC |
| | | | | VQYAQFPLTFGCGTKVEIKSGGGGQEVQLVES |
| | | | | GGGLVQPGGSLKLSCAASGFTFNKYAINWVRE |
| | | | | APGKGLEWVARIRSKYNNYATYYADAVKDRF |
| | | | | TISRDDSKNTAYLQMNNLKTEDTAVYYCVRN |
| | | | | ANFGTSYISYFAYWGQGTLVTVSSGGGGQGG |
| | | | | GGQGGGGQQTVVTQEPSLTVSPGGTVTLTCGS |
| | | | | STGAVTSGNYPNWVQKKPGQAPRGLIGGTKFL |
| | | | | APGTPARFSGSLLGGKAALTLSGVQPEDEAEY |
| | | | | YCVLWYSNRWVFGSGTKLTVLGGGGQGGGG |
| | | | | QGGGGQCPPCPAPELLGGPSVFLFPPKPKDTLM |
| | | | | ISRTPEVTCVVVDVSHEEPEVKFNWYVDGVEV |
| | | | | HNAKTKPCEEQYGSTYRCVSVLTVLHQDWLN |
| | | | | GKEYKCKVSNKALPAPIEKTISKAKGQPREPQV |
| | | | | YTLPPSREEMTKNQVSLTCLVKGFYPSDIAVE |
| | | | | WESNGQPENNYKTTPPVLDSDGSFFLYSKLTV |
| | | | | DKSRWQQGNVFSCSVMHEALHNHYTQKSLSL |
| | | | | SPGKGGGGQGGGGQGGGGQGGGGQGGGGQG |
| | | | | GGGQCPPCPAPELLGGPSVFLFPPKPKDTLMIS |
| | | | | RTPEVTCVVVDVSHEEPEVKFNWYVDGVEVH |
| | | | | NAKTKPCEEQYGSTYRCVSVLTVLHQDWLNG |
| | | | | KEYKCKVSNKALPAPIEKTISKAKGQPREPQVY |
| | | | | TLPPSREEMTKNQVSLTCLVKGFYPSDIAVEWE |
| | | | | SNGQPENNYKTTPPVLDSDGSFFLYSKLTVDKS |
| | | | | RWQQGNVFSCSVMHEALHNHYTQKSLSLSPG |
| | | | | KGGGGQGGGGQGGGGQQVQLQESGPGLVKPS |
| | | | | ETLSLTCTVSGGSISSSSYFWGWIRQPPGKCLE |
| | | | | WIGNIYYSGSSNYNPSLKSRVTISVDTSKNQFSL |
| | | | | KLSSVTAADTAVYYCARLPRGDRDAFDIWGQ |
| | | | | GTMVTVSSGGGGQGGGGQGGGGQDIVMTQSP |
| | | | | SSLSASVGDRVTITCRASQGISNYLAWYQQKP |
| | | | | GKVPKLLIYAASTLQSGVPSRFSGSGSGTDFTL |
| | | | | TISSLQPEDFATYYCQQSYSTPFTFGCGTKVEIK |
| | | | | SGGGGQEVQLVESGGGLVQPGGSLKLSCAASG |
| | | | | FTFNKYAINWVREAPGKGLEWVARIRSKYNN |
| | | | | YATYYADAVKDRFTISRDDSKNTAYLQMNNL |
| | | | | KTEDTAVYYCVRNANFGTSYISYFAYWGQGT |
| | | | | LVTVSSGGGGQGGGGQGGGGQQTVVTQEPSL |
| | | | | TVSPGGTVTLTCGSSTGAVTSGNYPNWVQKKP |
| | | | | GQAPRGLIGGTKFLAPGTPARFSGSLLGGKAAL |
| | | | | TLSGVQPEDEAEYYCVLWYSNRWVFGSGTKL |
| | | | | TVL |
| 256. | CH3 15-E11 CC x | artificial | Aa | QVQLVQSGAEVKKPGASVKVSCKASGYTFTN |
| | I2M2 x G4S3 x | | | YWMNWVRQAPGQCLEWMGNIAYGVKGTNY |
| | scFc x G4S3 x MS | | | NQKFQGRVTMTVDTSSSTAYMELSRLRSDDTA |
| | 15-B12 CC x | | | VYYCATRYFYVMDYWGQGTLVTVSSGGGGS |
| | I2M2 - Full | | | GGGGSGGGGSDIQMTQSPSSLSASVGDRVTITC |
| | Sequence | | | RASQDISNYLNWYQQKPGKVPKLLIYYTSRLH |
| | | | | SGVPSRFSGSGSGTDFTLTISSLQPEDVATYYCV |
| | | | | QYAQFPLTFGCGTKVEIKSGGGGSEVQLVESG |
| | | | | GGLVQPGGSLKLSCAASGFTFNKYAINWVREA |
| | | | | PGKGLEWVARIRSKYNNYATYYADAVKDRFTI |
| | | | | SRDDSKNTAYLQMNNLKTEDTAVYYCVRNAN |
| | | | | FGTSYISYFAYWGQGTLVTVSSGGGGSGGGGS |
| | | | | GGGGSQTVVTQEPSLTVSPGGTVTLTCGSSTG |
| | | | | AVTSGNYPNWVQKKPGQAPRGLIGGTKFLAPG |
| | | | | TPARFSGSLLGGKAALTLSGVQPEDEAEYYCV |
| | | | | LWYSNRWVFGSGTKLTVLGGGGSGGGGSGGG |
| | | | | GSDKTHTCPPCPAPELLGGPSVFLFPPKPKDTL |
| | | | | MISRTPEVTCVVVDVSHEDPEVKFNWYVDGV |
| | | | | EVHNAKTKPCEEQYGSTYRCVSVLTVLHQDW |
| | | | | LNGKEYKCKVSNKALPAPIEKTISKAKGQPREP |
| | | | | QVYTLPPSREEMTKNQVSLTCLVKGFYPSDIAV |
| | | | | EWESNGQPENNYKTTPPVLDSDGSFFLYSKLT |
| | | | | VDKSRWQQGNVFSCSVMHEALHNHYTQKSLS |
| | | | | LSPGKGGGGSGGGGSGGGGSGGGGSGGGGSG |
| | | | | GGGSDKTHTCPPCPAPELLGGPSVFLFPPKPKD |
| | | | | TLMISRTPEVTCVVVDVSHEDPEVKFNWYVDG |
| | | | | VEVHNAKTKPCEEQYGSTYRCVSVLTVLHQD |
| | | | | WLNGKEYKCKVSNKALPAPIEKTISKAKGQPR |
| | | | | EPQVYTLPPSREEMTKNQVSLTCLVKGFYPSDI |
| | | | | AVEWESNGQPENNYKTTPPVLDSDGSFFLYSK |
| | | | | LTVDKSRWQQGNVFSCSVMHEALHNHYTQKS |
| | | | | LSLSPGKGGGGSGGGGSGGGGSQVQLQESGPG |
| | | | | LVKPSETLSLTCTVSGGSISSSSYFWGWIRQPPG |
| | | | | KCLEWIGNIYYSGSSNYNPSLKSRVTISVDTSK |
| | | | | NQFSLKLSSVTAADTAVYYCARLPRGDRDAFD |
| | | | | IWGQGTMVTVSSGGGGSGGGGSGGGGSDIVM |
| | | | | TQSPSSLSASVGDRVTITCRASQGISNYLAWYQ |
| | | | | QKPGKVPKLLIYAASTLQSGVPSRFSGSGSGTD |
| | | | | FTLTISSLQPEDFATYYCQQSYSTPFTFGCGTKV |
| | | | | EIKSGGGGSEVQLVESGGGLVQPGGSLKLSCA |
| | | | | ASGFTFNKYAINWVREAPGKGLEWVARIRSKY |
| | | | | NNYATYYADAVKDRFTISRDDSKNTAYLQMN |
| | | | | NLKTEDTAVYYCVRNANFGTSYISYFAYWGQ |
| | | | | GTLVTVSSGGGGSGGGGSGGGGSQTVVTQEPS |
| | | | | LTVSPGGTVTLTCGSSTGAVTSGNYPNWVQKK |
| | | | | PGQAPRGLIGGTKFLAPGTPARFSGSLLGGKAA |
| | | | | LTLSGVQPEDEAEYYCVLWYSNRWVFGSGTK |
| | | | | LTVL |
| 257. | CH3 15-E11 CC x | artificial | Aa | QVQLVQSGAEVKKPGASVKVSCKASGYTFTN |
| | I2M2 x G4 x scFc | | | YWMNWVRQAPGQCLEWMGNIAYGVKGTNY |
| | x G4 x MS 15- | | | NQKFQGRVTMTVDTSSSTAYMELSRLRSDDTA |
| | B12 CC x I2M2 | | | VYYCATRYFYVMDYWGQGTLVTVSSGGGGQ |
| | _GQ - Full | | | GGGGQGGGGQDIQMTQSPSSLSASVGDRVTIT |
| | Sequence | | | CRASQDISNYLNWYQQKPGKVPKLLIYYTSRL |
| | | | | HSGVPSRFSGSGSGTDFTLTISSLQPEDVATYYC |
| | | | | VQYAQFPLTFGCGTKVEIKSGGGGQEVQLVES |
| | | | | GGGLVQPGGSLKLSCAASGFTFNKYAINWVRE |
| | | | | APGKGLEWVARIRSKYNNYATYYADAVKDRF |
| | | | | TISRDDSKNTAYLQMNNLKTEDTAVYYCVRN |
| | | | | ANFGTSYISYFAYWGQGTLVTVSSGGGGQGG |
| | | | | GGQGGGGQQTVVTQEPSLTVSPGGTVTLTCGS |
| | | | | STGAVTSGNYPNWVQKKPGQAPRGLIGGTKFL |
| | | | | APGTPARFSGSLLGGKAALTLSGVQPEDEAEY |
| | | | | YCVLWYSNRWVFGSGTKLTVLGGGGCPPCPA |
| | | | | PELLGGPSVFLFPPKPKDTLMISRTPEVTCVVV |
| | | | | DVSHEEPEVKFNWYVDGVEVHNAKTKPCEEQ |
| | | | | YGSTYRCVSVLTVLHQDWLNGKEYKCKVSNK |
| | | | | ALPAPIEKTISKAKGQPREPQVYTLPPSREEMT |
| | | | | KNQVSLTCLVKGFYPSDIAVEWESNGQPENNY |
| | | | | KTTPPVLDSDGSFFLYSKLTVDKSRWQQGNVF |
| | | | | SCSVMHEALHNHYTQKSLSLSPGKGGGGQGG |
| | | | | GGQGGGGQGGGGQGGGGQGGGGQCPPCPAP |
| | | | | ELLGGPSVFLFPPKPKDTLMISRTPEVTCVVVD |
| | | | | VSHEEPEVKFNWYVDGVEVHNAKTKPCEEQY |
| | | | | GSTYRCVSVLTVLHQDWLNGKEYKCKVSNKA |
| | | | | LPAPIEKTISKAKGQPREPQVYTLPPSREEMTK |
| | | | | NQVSLTCLVKGFYPSDIAVEWESNGQPENNYK |
| | | | | TTPPVLDSDGSFFLYSKLTVDKSRWQQGNVFS |
| | | | | CSVMHEALHNHYTQKSLSLSPGKGGGGQVQL |
| | | | | QESGPGLVKPSETLSLTCTVSGGSISSSSYFWG |
| | | | | WIRQPPGKCLEWIGNIYYSGSSNYNPSLKSRVT |
| | | | | ISVDTSKNQFSLKLSSVTAADTAVYYCARLPRG |
| | | | | DRDAFDIWGQGTMVTVSSGGGGQGGGGQGG |
| | | | | GGQDIVMTQSPSSLSASVGDRVTITCRASQGIS |
| | | | | NYLAWYQQKPGKVPKLLIYAASTLQSGVPSRF |
| | | | | SGSGSGTDFTLTISSLQPEDFATYYCQQSYSTPF |
| | | | | TFGCGTKVEIKSGGGGQEVQLVESGGGLVQPG |
| | | | | GSLKLSCAASGFTFNKYAINWVREAPGKGLEW |
| | | | | VARIRSKYNNYATYYADAVKDRFTISRDDSKN |
| | | | | TAYLQMNNLKTEDTAVYYCVRNANFGTSYIS |
| | | | | YFAYWGQGTLVTVSSGGGGQGGGGQGGGGQ |
| | | | | QTVVTQEPSLTVSPGGTVTLTCGSSTGAVTSGN |
| | | | | YPNWVQKKPGQAPRGLIGGTKFLAPGTPARFS |
| | | | | GSLLGGKAALTLSGVQPEDEAEYYCVLWYSN |
| | | | | RWVFGSGTKLTVL |
| 258. | CH3 15-E11 CCx | artificial | Aa | QVQLVQSGAEVKKPGASVKVSCKASGYTFTN |
| | I2C 44/100cc x | | | YWMNWVRQAPGQCLEWMGNIAYGVKGTNY |
| | scFc x MS 15-B12 | | | NQKFQGRVTMTVDTSSSTAYMELSRLRSDDTA |
| | CC x I2C | | | VYYCATRYFYVMDYWGQGTLVTVSSGGGGS |
| | 44/100cc0 - Full | | | GGGGSGGGGSDIQMTQSPSSLSASVGDRVTITC |
| | Sequence | | | RASQDISNYLNWYQQKPGKVPKLLIYYTSRLH |
| | | | | SGVPSRFSGSGSGTDFTLTISSLQPEDVATYYCV |
| | | | | QYAQFPLTFGCGTKVEIKSGGGGSEVQLVESG |
| | | | | GGLVQPGGSLKLSCAASGFTFNKYAMNWVRQ |
| | | | | APGKCLEWVARIRSKYNNYATYYADSVKDRF |
| | | | | TISRDDSKNTAYLQMNNLKTEDTAVYYCVRH |
| | | | | GNFGNSYISYWAYWGQGTLVTVSSGGGGSGG |
| | | | | GGSGGGGSQTVVTQEPSLTVSPGGTVTLTCGSS |
| | | | | TGAVTSGNYPNWVQQKPGQAPRGLIGGTKFL |
| | | | | APGTPARFSGSLLGGKAALTLSGVQPEDEAEY |
| | | | | YCVLWYSNRWVFGCGTKLTVLGGGGSGGGGS |
| | | | | GGGGSDKTHTCPPCPAPELLGGPSVFLFPPKPK |
| | | | | DTLMISRTPEVTCVVVDVSHEDPEVKFNWYVD |
| | | | | GVEVHNAKTKPCEEQYGSTYRCVSVLTVLHQ |
| | | | | DWLNGKEYKCKVSNKALPAPIEKTISKAKGQP |
| | | | | REPQVYTLPPSREEMTKNQVSLTCLVKGFYPS |
| | | | | DIAVEWESNGQPENNYKTTPPVLDSDGSFFLYS |
| | | | | KLTVDKSRWQQGNVFSCSVMHEALHNHYTQK |
| | | | | SLSLSPGKGGGGSGGGGSGGGGSGGGGSGGG |
| | | | | GSGGGGSDKTHTCPPCPAPELLGGPSVFLFPPK |
| | | | | PKDTLMISRTPEVTCVVVDVSHEDPEVKFNWY |
| | | | | VDGVEVHNAKTKPCEEQYGSTYRCVSVLTVL |
| | | | | HQDWLNGKEYKCKVSNKALPAPIEKTISKAKG |
| | | | | QPREPQVYTLPPSREEMTKNQVSLTCLVKGFY |
| | | | | PSDIAVEWESNGQPENNYKTTPPVLDSDGSFFL |
| | | | | YSKLTVDKSRWQQGNVFSCSVMHEALHNHYT |
| | | | | QKSLSLSPGKGGGGSGGGGSGGGGSQVQLQES |
| | | | | GPGLVKPSETLSLTCTVSGGSISSSSYFWGWIR |
| | | | | QPPGKCLEWIGNIYYSGSSNYNPSLKSRVTISV |
| | | | | DTSKNQFSLKLSSVTAADTAVYYCARLPRGDR |
| | | | | DAFDIWGQGTMVTVSSGGGGSGGGGSGGGGS |
| | | | | DIVMTQSPSSLSASVGDRVTITCRASQGISNYL |
| | | | | AWYQQKPGKVPKLLIYAASTLQSGVPSRFSGS |
| | | | | GSGTDFTLTISSLQPEDFATYYCQQSYSTPFTFG |
| | | | | CGTKVEIKSGGGGSEVQLVESGGGLVQPGGSL |
| | | | | KLSCAASGFTFNKYAMNWVRQAPGKCLEWV |
| | | | | ARIRSKYNNYATYYADSVKDRFTISRDDSKNT |
| | | | | AYLQMNNLKTEDTAVYYCVRHGNFGNSYISY |
| | | | | WAYWGQGTLVTVSSGGGGSGGGGSGGGGSQ |
| | | | | TVVTQEPSLTVSPGGTVTLTCGSSTGAVTSGNY |
| | | | | PNWVQQKPGQAPRGLIGGTKFLAPGTPARFSG |
| | | | | SLLGGKAALTLSGVQPEDEAEYYCVLWYSNR |
| | | | | WVFGCGTKLTVL |
| 259. | CH3 24-D7 CC x | artificial | Aa | QVQLVQSGAEVKKPGASVKVSCKASGYTFTN |
| | I2L x G4S3 x | | | YWMNWVRQAPGQCLEWMGNIHSKAHGTNY |
| | scFc x G4S3 x MS | | | NQKFQGRVTMTVDTSSSTAYMELSRLRSDDTA |
| | 15-B12 CC x I2L - | | | VYYCATRYFYVMDYWGQGTLVTVSSGGGGS |
| | Full Sequence | | | GGGGSGGGGSDIQMTQSPSSLSASVGDRVTITC |
| | | | | RASQDISNYLNWYQQKPGKVPKLLIYYTSRLH |
| | | | | SGVPSRFSGSGSGTDFTLTISSLQPEDVATYYCV |
| | | | | QYAQFPLTFGCGTKVEIKSGGGGSEVQLVESG |
| | | | | GGLVQPGGSLKLSCAASGFTFNKYAMNWVRQ |
| | | | | APGKGMEWVARIRSKYNNYATYYADAVKDR |
| | | | | FTISRDDSKNTLYLQMNNLKTEDTAVYYCVRA |
| | | | | GNFGSSYISYFAYWGQGTLVTVSSGGGGSGGG |
| | | | | GSGGGGSQTVVTQEPSLTVSPGGTVTITCGSST |
| | | | | GAVTSGNYPNWIQKKPGQAPRGLIGGTKFLAP |
| | | | | GTPARFSGSLEGGKAALTLSGVQPEDEAEYYC |
| | | | | VLYYSNRWVFGSGTKLTVLGGGGSGGGGSGG |
| | | | | GGSDKTHTCPPCPAPELLGGPSVFLFPPKPKDT |
| | | | | LMISRTPEVTCVVVDVSHEDPEVKFNWYVDG |
| | | | | VEVHNAKTKPCEEQYGSTYRCVSVLTVLHQD |
| | | | | WLNGKEYKCKVSNKALPAPIEKTISKAKGQPR |
| | | | | EPQVYTLPPSREEMTKNQVSLTCLVKGFYPSDI |
| | | | | AVEWESNGQPENNYKTTPPVLDSDGSFFLYSK |
| | | | | LTVDKSRWQQGNVFSCSVMHEALHNHYTQKS |
| | | | | LSLSPGKGGGGSGGGGSGGGGSGGGGSGGGG |
| | | | | SGGGGSDKTHTCPPCPAPELLGGPSVFLFPPKP |
| | | | | KDTLMISRTPEVTCVVVDVSHEDPEVKFNWYV |
| | | | | DGVEVHNAKTKPCEEQYGSTYRCVSVLTVLH |
| | | | | QDWLNGKEYKCKVSNKALPAPIEKTISKAKGQ |
| | | | | PREPQVYTLPPSREEMTKNQVSLTCLVKGFYPS |
| | | | | DIAVEWESNGQPENNYKTTPPVLDSDGSFFLYS |
| | | | | KLTVDKSRWQQGNVFSCSVMHEALHNHYTQK |
| | | | | SLSLSPGKGGGGSGGGGSGGGGSQVQLQESGP |
| | | | | GLVKPSETLSLTCTVSGGSISSSSYFWGWIRQPP |
| | | | | GKCLEWIGNIYYSGSSNYNPSLKSRVTISVDTS |
| | | | | KNQFSLKLSSVTAADTAVYYCARLPRGDRDAF |
| | | | | DIWGQGTMVTVSSGGGGSGGGGSGGGGSDIV |
| | | | | MTQSPSSLSASVGDRVTITCRASQGISNYLAWY |
| | | | | QQKPGKVPKLLIYAASTLQSGVPSRFSGSGSGT |
| | | | | DFTLTISSLQPEDFATYYCQQSYSTPFTFGCGTK |
| | | | | VEIKSGGGGSEVQLVESGGGLVQPGGSLKLSC |
| | | | | AASGFTFNKYAMNWVRQAPGKGMEWVARIR |
| | | | | SKYNNYATYYADAVKDRFTISRDDSKNTLYLQ |
| | | | | MNNLKTEDTAVYYCVRAGNFGSSYISYFAYW |
| | | | | GQGTLVTVSSGGGGSGGGGSGGGGSQTVVTQ |
| | | | | EPSLTVSPGGTVTITCGSSTGAVTSGNYPNWIQ |
| | | | | KKPGQAPRGLIGGTKFLAPGTPARFSGSLEGGK |
| | | | | AALTLSGVQPEDEAEYYCVLYYSNRWVFGSG |
| | | | | TKLTVL |
| 260. | CH3 24-D7 CC x | artificial | Aa | QVQLVQSGAEVKKPGASVKVSCKASGYTFTN |
| | I2L x G4 x scFc x | | | YWMNWVRQAPGQCLEWMGNIHSKAHGTNY |
| | G4 x MS15-B12 | | | NQKFQGRVTMTVDTSSSTAYMELSRLRSDDTA |
| | CC x I2L_GQ - | | | VYYCATRYFYVMDYWGQGTLVTVSSGGGGQ |
| | Full Sequence | | | GGGGQGGGGQDIQMTQSPSSLSASVGDRVTIT |
| | | | | CRASQDISNYLNWYQQKPGKVPKLLIYYTSRL |
| | | | | HSGVPSRFSGSGSGTDFTLTISSLQPEDVATYYC |
| | | | | VQYAQFPLTFGCGTKVEIKSGGGGQEVQLVES |
| | | | | GGGLVQPGGSLKLSCAASGFTFNKYAMNWVR |
| | | | | QAPGKGMEWVARIRSKYNNYATYYADAVKD |
| | | | | RFTISRDDSKNTLYLQMNNLKTEDTAVYYCVR |
| | | | | AGNFGSSYISYFAYWGQGTLVTVSSGGGGQGG |
| | | | | GGQGGGGQQTVVTQEPSLTVSPGGTVTITCGS |
| | | | | STGAVTSGNYPNWIQKKPGQAPRGLIGGTKFL |
| | | | | APGTPARFSGSLEGGKAALTLSGVQPEDEAEY |
| | | | | YCVLYYSNRWVFGSGTKLTVLGGGGCPPCPAP |
| | | | | ELLGGPSVFLFPPKPKDTLMISRTPEVTCVVVD |
| | | | | VSHEEPEVKFNWYVDGVEVHNAKTKPCEEQY |
| | | | | GSTYRCVSVLTVLHQDWLNGKEYKCKVSNKA |
| | | | | LPAPIEKTISKAKGQPREPQVYTLPPSREEMTK |
| | | | | NQVSLTCLVKGFYPSDIAVEWESNGQPENNYK |
| | | | | TTPPVLDSDGSFFLYSKLTVDKSRWQQGNVFS |
| | | | | CSVMHEALHNHYTQKSLSLSPGKGGGGQGGG |
| | | | | GQGGGGQGGGGQGGGGQGGGGQCPPCPAPEL |
| | | | | LGGPSVFLFPPKPKDTLMISRTPEVTCVVVDVS |
| | | | | HEEPEVKFNWYVDGVEVHNAKTKPCEEQYGS |
| | | | | TYRCVSVLTVLHQDWLNGKEYKCKVSNKALP |
| | | | | APIEKTISKAKGQPREPQVYTLPPSREEMTKNQ |
| | | | | VSLTCLVKGFYPSDIAVEWESNGQPENNYKTT |
| | | | | PPVLDSDGSFFLYSKLTVDKSRWQQGNVFSCS |
| | | | | VMHEALHNHYTQKSLSLSPGKGGGGQVQLQE |
| | | | | SGPGLVKPSETLSLTCTVSGGSISSSSYFWGWIR |
| | | | | QPPGKCLEWIGNIYYSGSSNYNPSLKSRVTISV |
| | | | | DTSKNQFSLKLSSVTAADTAVYYCARLPRGDR |
| | | | | DAFDIWGQGTMVTVSSGGGGQGGGGQGGGG |
| | | | | QDIVMTQSPSSLSASVGDRVTITCRASQGISNY |
| | | | | LAWYQQKPGKVPKLLIYAASTLQSGVPSRFSG |
| | | | | SGSGTDFTLTISSLQPEDFATYYCQQSYSTPFTF |
| | | | | GCGTKVEIKSGGGGQEVQLVESGGGLVQPGGS |
| | | | | LKLSCAASGFTFNKYAMNWVRQAPGKGMEW |
| | | | | VARIRSKYNNYATYYADAVKDRFTISRDDSKN |
| | | | | TLYLQMNNLKTEDTAVYYCVRAGNFGSSYISY |
| | | | | FAYWGQGTLVTVSSGGGGQGGGGQGGGGQQ |
| | | | | TVVTQEPSLTVSPGGTVTITCGSSTGAVTSGNY |
| | | | | PNWIQKKPGQAPRGLIGGTKFLAPGTPARFSGS |
| | | | | LEGGKAALTLSGVQPEDEAEYYCVLYYSNRW |
| | | | | VFGSGTKLTVL |
| 261. | CH3 24-D7 CC x | artificial | Aa | QVQLVQSGAEVKKPGASVKVSCKASGYTFTN |
| | I2M2 x G4 x scfc | | | YWMNWVRQAPGQCLEWMGNIHSKAHGTNY |
| | x G4 x MS 15- | | | NQKFQGRVTMTVDTSSSTAYMELSRLRSDDTA |
| | B12 CC x I2M2 - | | | VYYCATRYFYVMDYWGQGTLVTVSSGGGGS |
| | Full Sequence | | | GGGGSGGGGSDIQMTQSPSSLSASVGDRVTITC |
| | | | | RASQDISNYLNWYQQKPGKVPKLLIYYTSRLH |
| | | | | SGVPSRFSGSGSGTDFTLTISSLQPEDVATYYCV |
| | | | | QYAQFPLTFGCGTKVEIKSGGGGSEVQLVESG |
| | | | | GGLVQPGGSLKLSCAASGFTFNKYAINWVREA |
| | | | | PGKGLEWVARIRSKYNNYATYYADAVKDRFTI |
| | | | | SRDDSKNTAYLQMNNLKTEDTAVYYCVRNAN |
| | | | | FGTSYISYFAYWGQGTLVTVSSGGGGSGGGGS |
| | | | | GGGGSQTVVTQEPSLTVSPGGTVTLTCGSSTG |
| | | | | AVTSGNYPNWVQKKPGQAPRGLIGGTKFLAPG |
| | | | | TPARFSGSLLGGKAALTLSGVQPEDEAEYYCV |
| | | | | LWYSNRWVFGSGTKLTVLGGGGDKTHTCPPC |
| | | | | PAPELLGGPSVFLFPPKPKDTLMISRTPEVTCVV |
| | | | | VDVSHEDPEVKFNWYVDGVEVHNAKTKPCEE |
| | | | | QYGSTYRCVSVLTVLHQDWLNGKEYKCKVSN |
| | | | | KALPAPIEKTISKAKGQPREPQVYTLPPSREEM |
| | | | | TKNQVSLTCLVKGFYPSDIAVEWESNGQPENN |
| | | | | YKTTPPVLDSDGSFFLYSKLTVDKSRWQQGNV |
| | | | | FSCSVMHEALHNHYTQKSLSLSPGKGGGGSGG |
| | | | | GGSGGGGSGGGGSGGGGSGGGGSDKTHTCPP |
| | | | | CPAPELLGGPSVFLFPPKPKDTLMISRTPEVTCV |
| | | | | VVDVSHEDPEVKFNWYVDGVEVHNAKTKPCE |
| | | | | EQYGSTYRCVSVLTVLHQDWLNGKEYKCKVS |
| | | | | NKALPAPIEKTISKAKGQPREPQVYTLPPSREE |
| | | | | MTKNQVSLTCLVKGFYPSDIAVEWESNGQPEN |
| | | | | NYKTTPPVLDSDGSFFLYSKLTVDKSRWQQGN |
| | | | | VFSCSVMHEALHNHYTQKSLSLSPGKGGGGQ |
| | | | | VQLQESGPGLVKPSETLSLTCTVSGGSISSSSYF |
| | | | | WGWIRQPPGKCLEWIGNIYYSGSSNYNPSLKS |
| | | | | RVTISVDTSKNQFSLKLSSVTAADTAVYYCAR |
| | | | | LPRGDRDAFDIWGQGTMVTVSSGGGGSGGGG |
| | | | | SGGGGSDIVMTQSPSSLSASVGDRVTITCRASQ |
| | | | | GISNYLAWYQQKPGKVPKLLIYAASTLQSGVP |
| | | | | SRFSGSGSGTDFTLTISSLQPEDFATYYCQQSYS |
| | | | | TPFTFGCGTKVEIKSGGGGSEVQLVESGGGLV |
| | | | | QPGGSLKLSCAASGFTFNKYAINWVREAPGKG |
| | | | | LEWVARIRSKYNNYATYYADAVKDRFTISRDD |
| | | | | SKNTAYLQMNNLKTEDTAVYYCVRNANFGTS |
| | | | | YISYFAYWGQGTLVTVSSGGGGSGGGGSGGG |
| | | | | GSQTVVTQEPSLTVSPGGTVTLTCGSSTGAVTS |
| | | | | GNYPNWVQKKPGQAPRGLIGGTKFLAPGTPAR |
| | | | | FSGSLLGGKAALTLSGVQPEDEAEYYCVLWYS |
| | | | | NRWVFGSGTKLTVL |
| 262. | CH3 24-D7 CC x | artificial | aa | QVQLVQSGAEVKKPGASVKVSCKASGYTFTN |
| | G4 x scFc x G4 x | | | YWMNWVRQAPGQCLEWMGNIHSKAHGTNY |
| | MS 15-B12 CC x | | | NQKFQGRVTMTVDTSSSTAYMELSRLRSDDTA |
| | I2M2_GQ - Full | | | VYYCATRYFYVMDYWGQGTLVTVSSGGGGQ |
| | Sequence | | | GGGGQGGGGQDIQMTQSPSSLSASVGDRVTIT |
| | | | | CRASQDISNYLNWYQQKPGKVPKLLIYYTSRL |
| | | | | HSGVPSRFSGSGSGTDFTLTISSLQPEDVATYYC |
| | | | | VQYAQFPLTFGCGTKVEIKSGGGGQEVQLVES |
| | | | | GGGLVQPGGSLKLSCAASGFTFNKYAINWVRE |
| | | | | APGKGLEWVARIRSKYNNYATYYADAVKDRF |
| | | | | TISRDDSKNTAYLQMNNLKTEDTAVYYCVRN |
| | | | | ANFGTSYISYFAYWGQGTLVTVSSGGGGQGG |
| | | | | GGQGGGGQQTVVTQEPSLTVSPGGTVTLTCGS |
| | | | | STGAVTSGNYPNWVQKKPGQAPRGLIGGTKFL |
| | | | | APGTPARFSGSLLGGKAALTLSGVQPEDEAEY |
| | | | | YCVLWYSNRWVFGSGTKLTVLGGGGCPPCPA |
| | | | | PELLGGPSVFLFPPKPKDTLMISRTPEVTCVVV |
| | | | | DVSHEEPEVKFNWYVDGVEVHNAKTKPCEEQ |
| | | | | YGSTYRCVSVLTVLHQDWLNGKEYKCKVSNK |
| | | | | ALPAPIEKTISKAKGQPREPQVYTLPPSREEMT |
| | | | | KNQVSLTCLVKGFYPSDIAVEWESNGQPENNY |
| | | | | KTTPPVLDSDGSFFLYSKLTVDKSRWQQGNVF |
| | | | | SCSVMHEALHNHYTQKSLSLSPGKGGGGQGG |
| | | | | GGQGGGGQGGGGQGGGGQGGGGQCPPCPAP |
| | | | | ELLGGPSVFLFPPKPKDTLMISRTPEVTCVVVD |
| | | | | VSHEEPEVKFNWYVDGVEVHNAKTKPCEEQY |
| | | | | GSTYRCVSVLTVLHQDWLNGKEYKCKVSNKA |
| | | | | LPAPIEKTISKAKGQPREPQVYTLPPSREEMTK |
| | | | | NQVSLTCLVKGFYPSDIAVEWESNGQPENNYK |
| | | | | TTPPVLDSDGSFFLYSKLTVDKSRWQQGNVFS |
| | | | | CSVMHEALHNHYTQKSLSLSPGKGGGGQVQL |
| | | | | QESGPGLVKPSETLSLTCTVSGGSISSSSYFWG |
| | | | | WIRQPPGKCLEWIGNIYYSGSSNYNPSLKSRVT |
| | | | | ISVDTSKNQFSLKLSSVTAADTAVYYCARLPRG |
| | | | | DRDAFDIWGQGTMVTVSSGGGGQGGGGQGG |
| | | | | GGQDIVMTQSPSSLSASVGDRVTITCRASQGIS |
| | | | | NYLAWYQQKPGKVPKLLIYAASTLQSGVPSRF |
| | | | | SGSGSGTDFTLTISSLQPEDFATYYCQQSYSTPF |
| | | | | TFGCGTKVEIKSGGGGQEVQLVESGGGLVQPG |
| | | | | GSLKLSCAASGFTFNKYAINWVREAPGKGLEW |
| | | | | VARIRSKYNNYATYYADAVKDRFTISRDDSKN |
| | | | | TAYLQMNNLKTEDTAVYYCVRNANFGTSYIS |
| | | | | YFAYWGQGTLVTVSSGGGGQGGGGQGGGGQ |
| | | | | QTVVTQEPSLTVSPGGTVTLTCGSSTGAVTSGN |
| | | | | YPNWVQKKPGQAPRGLIGGTKFLAPGTPARFS |
| | | | | GSLLGGKAALTLSGVQPEDEAEYYCVLWYSN |
| | | | | RWVFGSGTKLTVL |
| 263. | CH3 24-D7 CC x | artificial | aa | QVQLVQSGAEVKKPGASVKVSCKASGYTFTN |
| | I2L x (G4Q)3x | | | YWMNWVRQAPGQCLEWMGNIHSKAHGTNY |
| | scFcmod x | | | NQKFQGRVTMTVDTSSSTAYMELSRLRSDDTA |
| | (G4Q)3 x MS 15- | | | VYYCATRYFYVMDYWGQGTLVTVSSGGGGQ |
| | B12 CC x I2L - | | | GGGGQGGGGQDIQMTQSPSSLSASVGDRVTIT |
| | Full Sequence | | | CRASQDISNYLNWYQQKPGKVPKLLIYYTSRL |
| | | | | HSGVPSRFSGSGSGTDFTLTISSLQPEDVATYYC |
| | | | | VQYAQFPLTFGCGTKVEIKSGGGGQEVQLVES |
| | | | | GGGLVQPGGSLKLSCAASGFTFNKYAMNWVR |
| | | | | QAPGKGMEWVARIRSKYNNYATYYADAVKD |
| | | | | RFTISRDDSKNTLYLQMNNLKTEDTAVYYCVR |
| | | | | AGNFGSSYISYFAYWGQGTLVTVSSGGGGQGG |
| | | | | GGQGGGGQQTVVTQEPSLTVSPGGTVTITCGS |
| | | | | STGAVTSGNYPNWIQKKPGQAPRGLIGGTKFL |
| | | | | APGTPARFSGSLEGGKAALTLSGVQPEDEAEY |
| | | | | YCVLYYSNRWVFGSGTKLTVLGGGGQGGGGQ |
| | | | | GGGGQCPPCPAPELLGGPSVFLFPPKPKDTLMI |
| | | | | SRTPEVTCVVVDVSHEEPEVKFNWYVDGVEV |
| | | | | HNAKTKPCEEQYGSTYRCVSVLTVLHQDWLN |
| | | | | GKEYKCKVSNKALPAPIEKTISKAKGQPREPQV |
| | | | | YTLPPSREEMTKNQVSLTCLVKGFYPSDIAVE |
| | | | | WESNGQPENNYKTTPPVLDSDGSFFLYSKLTV |
| | | | | DKSRWQQGNVFSCSVMHEALHNHYTQKSLSL |
| | | | | SPGKGGGGQGGGGQGGGGQGGGGQGGGGQG |
| | | | | GGGQCPPCPAPELLGGPSVFLFPPKPKDTLMIS |
| | | | | RTPEVTCVVVDVSHEEPEVKFNWYVDGVEVH |
| | | | | NAKTKPCEEQYGSTYRCVSVLTVLHQDWLNG |
| | | | | KEYKCKVSNKALPAPIEKTISKAKGQPREPQVY |
| | | | | TLPPSREEMTKNQVSLTCLVKGFYPSDIAVEWE |
| | | | | SNGQPENNYKTTPPVLDSDGSFFLYSKLTVDKS |
| | | | | RWQQGNVFSCSVMHEALHNHYTQKSLSLSPG |
| | | | | KGGGGQGGGGQGGGGQQVQLQESGPGLVKPS |
| | | | | ETLSLTCTVSGGSISSSSYFWGWIRQPPGKCLE |
| | | | | WIGNIYYSGSSNYNPSLKSRVTISVDTSKNQFSL |
| | | | | KLSSVTAADTAVYYCARLPRGDRDAFDIWGQ |
| | | | | GTMVTVSSGGGGQGGGGQGGGGQDIVMTQSP |
| | | | | SSLSASVGDRVTITCRASQGISNYLAWYQQKP |
| | | | | GKVPKLLIYAASTLQSGVPSRFSGSGSGTDFTL |
| | | | | TISSLQPEDFATYYCQQSYSTPFTFGCGTKVEIK |
| | | | | SGGGGQEVQLVESGGGLVQPGGSLKLSCAASG |
| | | | | FTFNKYAMNWVRQAPGKGMEWVARIRSKYN |
| | | | | NYATYYADAVKDRFTISRDDSKNTLYLQMNN |
| | | | | LKTEDTAVYYCVRAGNFGSSYISYFAYWGQGT |
| | | | | LVTVSSGGGGQGGGGQGGGGQQTVVTQEPSL |
| | | | | TVSPGGTVTITCGSSTGAVTSGNYPNWIQKKPG |
| | | | | QAPRGLIGGTKFLAPGTPARFSGSLEGGKAALT |
| | | | | LSGVQPEDEAEYYCVLYYSNRWVFGSGTKLT |
| | | | | VL |
| 264. | CH3 24-D7 CC x | artificial | aa | QVQLVQSGAEVKKPGASVKVSCKASGYTFTN |
| | I2L x G4 x scFc x | | | YWMNWVRQAPGQCLEWMGNIHSKAHGTNY |
| | G4 x MS 15-B12 | | | NQKFQGRVTMTVDTSSSTAYMELSRLRSDDTA |
| | CC x I2L - Full | | | VYYCATRYFYVMDYWGQGTLVTVSSGGGGS |
| | Sequence | | | GGGGSGGGGSDIQMTQSPSSLSASVGDRVTITC |
| | | | | RASQDISNYLNWYQQKPGKVPKLLIYYTSRLH |
| | | | | SGVPSRFSGSGSGTDFTLTISSLQPEDVATYYCV |
| | | | | QYAQFPLTFGCGTKVEIKSGGGGSEVQLVESG |
| | | | | GGLVQPGGSLKLSCAASGFTFNKYAMNWVRQ |
| | | | | APGKGMEWVARIRSKYNNYATYYADAVKDR |
| | | | | FTISRDDSKNTLYLQMNNLKTEDTAVYYCVRA |
| | | | | GNFGSSYISYFAYWGQGTLVTVSSGGGGSGGG |
| | | | | GSGGGGSQTVVTQEPSLTVSPGGTVTITCGSST |
| | | | | GAVTSGNYPNWIQKKPGQAPRGLIGGTKFLAP |
| | | | | GTPARFSGSLEGGKAALTLSGVQPEDEAEYYC |
| | | | | VLYYSNRWVFGSGTKLTVLGGGGDKTHTCPP |
| | | | | CPAPELLGGPSVFLFPPKPKDTLMISRTPEVTCV |
| | | | | VVDVSHEDPEVKFNWYVDGVEVHNAKTKPCE |
| | | | | EQYGSTYRCVSVLTVLHQDWLNGKEYKCKVS |
| | | | | NKALPAPIEKTISKAKGQPREPQVYTLPPSREE |
| | | | | MTKNQVSLTCLVKGFYPSDIAVEWESNGQPEN |
| | | | | NYKTTPPVLDSDGSFFLYSKLTVDKSRWQQGN |
| | | | | VFSCSVMHEALHNHYTQKSLSLSPGKGGGGSG |
| | | | | GGGSGGGGSGGGGSGGGGSGGGGSDKTHTCP |
| | | | | PCPAPELLGGPSVFLFPPKPKDTLMISRTPEVTC |
| | | | | VVVDVSHEDPEVKFNWYVDGVEVHNAKTKPC |
| | | | | EEQYGSTYRCVSVLTVLHQDWLNGKEYKCKV |
| | | | | SNKALPAPIEKTISKAKGQPREPQVYTLPPSREE |
| | | | | MTKNQVSLTCLVKGFYPSDIAVEWESNGQPEN |
| | | | | NYKTTPPVLDSDGSFFLYSKLTVDKSRWQQGN |
| | | | | VFSCSVMHEALHNHYTQKSLSLSPGKGGGGQ |
| | | | | VQLQESGPGLVKPSETLSLTCTVSGGSISSSSYF |
| | | | | WGWIRQPPGKCLEWIGNIYYSGSSNYNPSLKS |
| | | | | RVTISVDTSKNQFSLKLSSVTAADTAVYYCAR |
| | | | | LPRGDRDAFDIWGQGTMVTVSSGGGGSGGGG |
| | | | | SGGGGSDIVMTQSPSSLSASVGDRVTITCRASQ |
| | | | | GISNYLAWYQQKPGKVPKLLIYAASTLQSGVP |
| | | | | SRFSGSGSGTDFTLTISSLQPEDFATYYCQQSYS |
| | | | | TPFTFGCGTKVEIKSGGGGSEVQLVESGGGLV |
| | | | | QPGGSLKLSCAASGFTFNKYAMNWVRQAPGK |
| | | | | GMEWVARIRSKYNNYATYYADAVKDRFTISR |
| | | | | DDSKNTLYLQMNNLKTEDTAVYYCVRAGNFG |
| | | | | SSYISYFAYWGQGTLVTVSSGGGGSGGGGSGG |
| | | | | GGSQTVVTQEPSLTVSPGGTVTITCGSSTGAVT |
| | | | | SGNYPNWIQKKPGQAPRGLIGGTKFLAPGTPA |
| | | | | RFSGSLEGGKAALTLSGVQPEDEAEYYCVLYY |
| | | | | SNRWVFGSGTKLTVL |
| 265. | CH3 24-D7 CC x | artificial | aa | QVQLVQSGAEVKKPGASVKVSCKASGYTFTN |
| | I2M2 x (G4Q)3x | | | YWMNWVRQAPGQCLEWMGNIHSKAHGTNY |
| | scFcmod x | | | NQKFQGRVTMTVDTSSSTAYMELSRLRSDDTA |
| | (G4Q)3 x MS 15- | | | VYYCATRYFYVMDYWGQGTLVTVSSGGGGQ |
| | B12 CC x I2M2 - | | | GGGGQGGGGQDIQMTQSPSSLSASVGDRVTIT |
| | Full Sequence | | | CRASQDISNYLNWYQQKPGKVPKLLIYYTSRL |
| | | | | HSGVPSRFSGSGSGTDFTLTISSLQPEDVATYYC |
| | | | | VQYAQFPLTFGCGTKVEIKSGGGGQEVQLVES |
| | | | | GGGLVQPGGSLKLSCAASGFTFNKYAINWVRE |
| | | | | APGKGLEWVARIRSKYNNYATYYADAVKDRF |
| | | | | TISRDDSKNTAYLQMNNLKTEDTAVYYCVRN |
| | | | | ANFGTSYISYFAYWGQGTLVTVSSGGGGQGG |
| | | | | GGQGGGGQQTVVTQEPSLTVSPGGTVTLTCGS |
| | | | | STGAVTSGNYPNWVQKKPGQAPRGLIGGTKFL |
| | | | | APGTPARFSGSLLGGKAALTLSGVQPEDEAEY |
| | | | | YCVLWYSNRWVFGSGTKLTVLGGGGQGGGG |
| | | | | QGGGGQCPPCPAPELLGGPSVFLFPPKPKDTLM |
| | | | | ISRTPEVTCVVVDVSHEEPEVKFNWYVDGVEV |
| | | | | HNAKTKPCEEQYGSTYRCVSVLTVLHQDWLN |
| | | | | GKEYKCKVSNKALPAPIEKTISKAKGQPREPQV |
| | | | | YTLPPSREEMTKNQVSLTCLVKGFYPSDIAVE |
| | | | | WESNGQPENNYKTTPPVLDSDGSFFLYSKLTV |
| | | | | DKSRWQQGNVFSCSVMHEALHNHYTQKSLSL |
| | | | | SPGKGGGGQGGGGQGGGGQGGGGQGGGGQG |
| | | | | GGGQCPPCPAPELLGGPSVFLFPPKPKDTLMIS |
| | | | | RTPEVTCVVVDVSHEEPEVKFNWYVDGVEVH |
| | | | | NAKTKPCEEQYGSTYRCVSVLTVLHQDWLNG |
| | | | | KEYKCKVSNKALPAPIEKTISKAKGQPREPQVY |
| | | | | TLPPSREEMTKNQVSLTCLVKGFYPSDIAVEWE |
| | | | | SNGQPENNYKTTPPVLDSDGSFFLYSKLTVDKS |
| | | | | RWQQGNVFSCSVMHEALHNHYTQKSLSLSPG |
| | | | | KGGGGQGGGGQGGGGQQVQLQESGPGLVKPS |
| | | | | ETLSLTCTVSGGSISSSSYFWGWIRQPPGKCLE |
| | | | | WIGNIYYSGSSNYNPSLKSRVTISVDTSKNQFSL |
| | | | | KLSSVTAADTAVYYCARLPRGDRDAFDIWGQ |
| | | | | GTMVTVSSGGGGQGGGGQGGGGQDIVMTQSP |
| | | | | SSLSASVGDRVTITCRASQGISNYLAWYQQKP |
| | | | | GKVPKLLIYAASTLQSGVPSRFSGSGSGTDFTL |
| | | | | TISSLQPEDFATYYCQQSYSTPFTFGCGTKVEIK |
| | | | | SGGGGQEVQLVESGGGLVQPGGSLKLSCAASG |
| | | | | FTFNKYAINWVREAPGKGLEWVARIRSKYNN |
| | | | | YATYYADAVKDRFTISRDDSKNTAYLQMNNL |
| | | | | KTEDTAVYYCVRNANFGTSYISYFAYWGQGT |
| | | | | LVTVSSGGGGQGGGGQGGGGQQTVVTQEPSL |
| | | | | TVSPGGTVTLTCGSSTGAVTSGNYPNWVQKKP |
| | | | | GQAPRGLIGGTKFLAPGTPARFSGSLLGGKAAL |
| | | | | TLSGVQPEDEAEYYCVLWYSNRWVFGSGTKL |
| | | | | TVL |
| 266. | CH3 24-D7 CC x | artificial | aa | QVQLVQSGAEVKKPGASVKVSCKASGYTFTN |
| | I2M2 x G4S3 x | | | YWMNWVRQAPGQCLEWMGNIHSKAHGTNY |
| | scFc x G4S3 x | | | NQKFQGRVTMTVDTSSSTAYMELSRLRSDDTA |
| | MS 15-B12 CC x | | | VYYCATRYFYVMDYWGQGTLVTVSSGGGGS |
| | I2M2 - Full | | | GGGGSGGGGSDIQMTQSPSSLSASVGDRVTITC |
| | Sequence | | | RASQDISNYLNWYQQKPGKVPKLLIYYTSRLH |
| | | | | SGVPSRFSGSGSGTDFTLTISSLQPEDVATYYCV |
| | | | | QYAQFPLTFGCGTKVEIKSGGGGSEVQLVESG |
| | | | | GGLVQPGGSLKLSCAASGFTFNKYAINWVREA |
| | | | | PGKGLEWVARIRSKYNNYATYYADAVKDRFTI |
| | | | | SRDDSKNTAYLQMNNLKTEDTAVYYCVRNAN |
| | | | | FGTSYISYFAYWGQGTLVTVSSGGGGSGGGGS |
| | | | | GGGGSQTVVTQEPSLTVSPGGTVTLTCGSSTG |
| | | | | AVTSGNYPNWVQKKPGQAPRGLIGGTKFLAPG |
| | | | | TPARFSGSLLGGKAALTLSGVQPEDEAEYYCV |
| | | | | LWYSNRWVFGSGTKLTVLGGGGSGGGGSGGG |
| | | | | GSDKTHTCPPCPAPELLGGPSVFLFPPKPKDTL |
| | | | | MISRTPEVTCVVVDVSHEDPEVKFNWYVDGV |
| | | | | EVHNAKTKPCEEQYGSTYRCVSVLTVLHQDW |
| | | | | LNGKEYKCKVSNKALPAPIEKTISKAKGQPREP |
| | | | | QVYTLPPSREEMTKNQVSLTCLVKGFYPSDIAV |
| | | | | EWESNGQPENNYKTTPPVLDSDGSFFLYSKLT |
| | | | | VDKSRWQQGNVFSCSVMHEALHNHYTQKSLS |
| | | | | LSPGKGGGGSGGGGSGGGGSGGGGSGGGGSG |
| | | | | GGGSDKTHTCPPCPAPELLGGPSVFLFPPKPKD |
| | | | | TLMISRTPEVTCVVVDVSHEDPEVKFNWYVDG |
| | | | | VEVHNAKTKPCEEQYGSTYRCVSVLTVLHQD |
| | | | | WLNGKEYKCKVSNKALPAPIEKTISKAKGQPR |
| | | | | EPQVYTLPPSREEMTKNQVSLTCLVKGFYPSDI |
| | | | | AVEWESNGQPENNYKTTPPVLDSDGSFFLYSK |
| | | | | LTVDKSRWQQGNVFSCSVMHEALHNHYTQKS |
| | | | | LSLSPGKGGGGSGGGGSGGGGSQVQLQESGPG |
| | | | | LVKPSETLSLTCTVSGGSISSSSYFWGWIRQPPG |
| | | | | KCLEWIGNIYYSGSSNYNPSLKSRVTISVDTSK |
| | | | | NQFSLKLSSVTAADTAVYYCARLPRGDRDAFD |
| | | | | IWGQGTMVTVSSGGGGSGGGGSGGGGSDIVM |
| | | | | TQSPSSLSASVGDRVTITCRASQGISNYLAWYQ |
| | | | | QKPGKVPKLLIYAASTLQSGVPSRFSGSGSGTD |
| | | | | FTLTISSLQPEDFATYYCQQSYSTPFTFGCGTKV |
| | | | | EIKSGGGGSEVQLVESGGGLVQPGGSLKLSCA |
| | | | | ASGFTFNKYAINWVREAPGKGLEWVARIRSKY |
| | | | | NNYATYYADAVKDRFTISRDDSKNTAYLQMN |
| | | | | NLKTEDTAVYYCVRNANFGTSYISYFAYWGQ |
| | | | | GTLVTVSSGGGGSGGGGSGGGGSQTVVTQEPS |
| | | | | LTVSPGGTVTLTCGSSTGAVTSGNYPNWVQKK |
| | | | | PGQAPRGLIGGTKFLAPGTPARFSGSLLGGKAA |
| | | | | LTLSGVQPEDEAEYYCVLWYSNRWVFGSGTK |
| | | | | LTVL |
| 267. | CH3 24-D7 CCx | artificial | aa | QVQLVQSGAEVKKPGASVKVSCKASGYTFTN |
| | 6H10.09x | | | YWMNWVRQAPGQCLEWMGNIHSKAHGTNY |
| | (G4S)3x scFcx | | | NQKFQGRVTMTVDTSSSTAYMELSRLRSDDTA |
| | (G4S)3x MS R4L | | | VYYCATRYFYVMDYWGQGTLVTVSSGGGGS |
| | CCx 6H10.09 - | | | GGGGSGGGGSDIQMTQSPSSLSASVGDRVTITC |
| | Full Sequence | | | RASQDISNYLNWYQQKPGKVPKLLIYYTSRLH |
| | | | | SGVPSRFSGSGSGTDFTLTISSLQPEDVATYYCV |
| | | | | QYAQFPLTFGCGTKVEIKSGGGGSEVQLVESG |
| | | | | GGLVQPGGSLKLSCAASGFTFNKYAMNWVRQ |
| | | | | APGKGMEWVARIRSKYNNYATYYADAVKDR |
| | | | | FTISRDDSKNTLYLQMNNLKTEDTAVYYCVRA |
| | | | | GNFGSSYISYFAYWGQGTLVTVSSGGGGSGGG |
| | | | | GSGGGGSQTVVTQEPSLTVSPGGTVTITCGSST |
| | | | | GAVTSGNYPNWIQKKPGQAPRGLIGGTKFLAP |
| | | | | GTPARFSGSLEGGKAALTLSGVQPEDEAEYYC |
| | | | | VLYYSNRWVFGSGTKLTVLGGGGSGGGGSGG |
| | | | | GGSDKTHTCPPCPAPELLGGPSVFLFPPKPKDT |
| | | | | LMISRTPEVTCVVVDVSHEDPEVKFNWYVDG |
| | | | | VEVHNAKTKPCEEQYGSTYRCVSVLTVLHQD |
| | | | | WLNGKEYKCKVSNKALPAPIEKTISKAKGQPR |
| | | | | EPQVYTLPPSREEMTKNQVSLTCLVKGFYPSDI |
| | | | | AVEWESNGQPENNYKTTPPVLDSDGSFFLYSK |
| | | | | LTVDKSRWQQGNVFSCSVMHEALHNHYTQKS |
| | | | | LSLSPGKGGGGSGGGGSGGGGSGGGGSGGGG |
| | | | | SGGGGSDKTHTCPPCPAPELLGGPSVFLFPPKP |
| | | | | KDTLMISRTPEVTCVVVDVSHEDPEVKFNWYV |
| | | | | DGVEVHNAKTKPCEEQYGSTYRCVSVLTVLH |
| | | | | QDWLNGKEYKCKVSNKALPAPIEKTISKAKGQ |
| | | | | PREPQVYTLPPSREEMTKNQVSLTCLVKGFYPS |
| | | | | DIAVEWESNGQPENNYKTTPPVLDSDGSFFLYS |
| | | | | KLTVDKSRWQQGNVFSCSVMHEALHNHYTQK |
| | | | | SLSLSPGKGGGGSGGGGSGGGGSQVQLVQSGA |
| | | | | EVKKPGASVKVSCKASGYTFTGYYIHWVRQA |
| | | | | PGQCLEWMGWINPNSGGTNYAQKFQGRVTMT |
| | | | | RDTSISTAYMELSRLRSDDTAVYYCARVEAVA |
| | | | | GREYYYFSGMDVWGQGTTVTVSSGGGGSGGG |
| | | | | GSGGGGSSYELTQPPSVSVSPGQTASITCSGEK |
| | | | | LGDKYVYWYQQKPGQSPVLVIYQSTKRPSGVP |
| | | | | ERFSGSNSGNTATLTISGTQAMDEADYYCQAY |
| | | | | HASTAVFGCGTKLTVLSGGGGSEVQLVESGGG |
| | | | | LVQPGGSLKLSCAASGFTFNKYAMNWVRQAP |
| | | | | GKGMEWVARIRSKYNNYATYYADAVKDRFTI |
| | | | | SRDDSKNTLYLQMNNLKTEDTAVYYCVRAGN |
| | | | | FGSSYISYFAYWGQGTLVTVSSGGGGSGGGGS |
| | | | | GGGGSQTVVTQEPSLTVSPGGTVTITCGSSTGA |
| | | | | VTSGNYPNWIQKKPGQAPRGLIGGTKFLAPGT |
| | | | | PARFSGSLEGGKAALTLSGVQPEDEAEYYCVL |
| | | | | YYSNRWVFGSGTKLTVL |
| 268. | CH3 R164L CC x | artificial | aa | EVQLLESGGGLVQPGGSVRLSCAASGFTESSY |
| | I2Ccc(44/100)x | | | WMYWVRQAPGKCLEWVSKIDPSDDYTNYNQ |
| | (G4S)3x scFcx | | | KVKGRFTISIDNSKNTLYLQMNSLRAEDSAVY |
| | (G4S)3x MS R4L | | | YCARWDYTHFDVWGQGTTVTVSSGGGGSGG |
| | CCx | | | GGSGGGGSEIVMTQSPATLSVSPGERATLSCRA |
| | I2Ccc(44/100) - | | | SSSVSYMHWYQQKPGQAPRLLIYGTSNLASGV |
| | Full Sequence | | | PVRFSGSGSGTEFTLTISRLQSEDVAVYYCQQW |
| | | | | SSYPLTFGCGTKVEIKSGGGGSEVQLVESGGGL |
| | | | | VQPGGSLKLSCAASGFTFNKYAMNWVRQAPG |
| | | | | KCLEWVARIRSKYNNYATYYADSVKDRFTISR |
| | | | | DDSKNTAYLQMNNLKTEDTAVYYCVRHGNFG |
| | | | | NSYISYWAYWGQGTLVTVSSGGGGSGGGGSG |
| | | | | GGGSQTVVTQEPSLTVSPGGTVTLTCGSSTGA |
| | | | | VTSGNYPNWVQQKPGQAPRGLIGGTKFLAPGT |
| | | | | PARFSGSLLGGKAALTLSGVQPEDEAEYYCVL |
| | | | | WYSNRWVFGCGTKLTVLGGGGSGGGGSGGG |
| | | | | GSDKTHTCPPCPAPELLGGPSVFLFPPKPKDTL |
| | | | | MISRTPEVTCVVVDVSHEDPEVKFNWYVDGV |
| | | | | EVHNAKTKPCEEQYGSTYRCVSVLTVLHQDW |
| | | | | LNGKEYKCKVSNKALPAPIEKTISKAKGQPREP |
| | | | | QVYTLPPSREEMTKNQVSLTCLVKGFYPSDIAV |
| | | | | EWESNGQPENNYKTTPPVLDSDGSFFLYSKLT |
| | | | | VDKSRWQQGNVFSCSVMHEALHNHYTQKSLS |
| | | | | LSPGKGGGGSGGGGSGGGGSGGGGSGGGGSG |
| | | | | GGGSDKTHTCPPCPAPELLGGPSVFLFPPKPKD |
| | | | | TLMISRTPEVTCVVVDVSHEDPEVKFNWYVDG |
| | | | | VEVHNAKTKPCEEQYGSTYRCVSVLTVLHQD |
| | | | | WLNGKEYKCKVSNKALPAPIEKTISKAKGQPR |
| | | | | EPQVYTLPPSREEMTKNQVSLTCLVKGFYPSDI |
| | | | | AVEWESNGQPENNYKTTPPVLDSDGSFFLYSK |
| | | | | LTVDKSRWQQGNVFSCSVMHEALHNHYTQKS |
| | | | | LSLSPGKGGGGSGGGGSGGGGSQVQLVQSGA |
| | | | | EVKKPGASVKVSCKASGYTFTGYYIHWVRQA |
| | | | | PGQCLEWMGWINPNSGGTNYAQKFQGRVTMT |
| | | | | RDTSISTAYMELSRLRSDDTAVYYCARVEAVA |
| | | | | GREYYYFSGMDVWGQGTTVTVSSGGGGSGGG |
| | | | | GSGGGGSSYELTQPPSVSVSPGQTASITCSGEK |
| | | | | LGDKYVYWYQQKPGQSPVLVIYQSTKRPSGVP |
| | | | | ERFSGSNSGNTATLTISGTQAMDEADYYCQAY |
| | | | | HASTAVFGCGTKLTVLSGGGGSEVQLVESGGG |
| | | | | LVQPGGSLKLSCAASGFTFNKYAMNWVRQAP |
| | | | | GKCLEWVARIRSKYNNYATYYADSVKDRFTIS |
| | | | | RDDSKNTAYLQMNNLKTEDTAVYYCVRHGNF |
| | | | | GNSYISYWAYWGQGTLVTVSSGGGGSGGGGS |
| | | | | GGGGSQTVVTQEPSLTVSPGGTVTLTCGSSTG |
| | | | | AVTSGNYPNWVQQKPGQAPRGLIGGTKFLAPG |
| | | | | TPARFSGSLLGGKAALTLSGVQPEDEAEYYCV |
| | | | | LWYSNRWVFGCGTKLTVL |
| 269. | CH3 R164L CCx | artificial | aa | EVQLLESGGGLVQPGGSVRLSCAASGFTESSY |
| | 6H10.09x | | | WMYWVRQAPGKCLEWVSKIDPSDDYTNYNQ |
| | (G4S)3x scFcx | | | KVKGRFTISIDNSKNTLYLQMNSLRAEDSAVY |
| | (G4S)3x MS R4L | | | YCARWDYTHFDVWGQGTTVTVSSGGGGSGG |
| | CCx 6H10.09 - | | | GGSGGGGSEIVMTQSPATLSVSPGERATLSCRA |
| | Full Sequence | | | SSSVSYMHWYQQKPGQAPRLLIYGTSNLASGV |
| | | | | PVRFSGSGSGTEFTLTISRLQSEDVAVYYCQQW |
| | | | | SSYPLTFGCGTKVEIKSGGGGSEVQLVESGGGL |
| | | | | VQPGGSLKLSCAASGFTFNKYAMNWVRQAPG |
| | | | | KGMEWVARIRSKYNNYATYYADAVKDRFTIS |
| | | | | RDDSKNTLYLQMNNLKTEDTAVYYCVRAGNF |
| | | | | GSSYISYFAYWGQGTLVTVSSGGGGSGGGGSG |
| | | | | GGGSQTVVTQEPSLTVSPGGTVTITCGSSTGAV |
| | | | | TSGNYPNWIQKKPGQAPRGLIGGTKFLAPGTP |
| | | | | ARFSGSLEGGKAALTLSGVQPEDEAEYYCVLY |
| | | | | YSNRWVFGSGTKLTVLGGGGSGGGGSGGGGS |
| | | | | DKTHTCPPCPAPELLGGPSVFLFPPKPKDTLMIS |
| | | | | RTPEVTCVVVDVSHEDPEVKFNWYVDGVEVH |
| | | | | NAKTKPCEEQYGSTYRCVSVLTVLHQDWLNG |
| | | | | KEYKCKVSNKALPAPIEKTISKAKGQPREPQVY |
| | | | | TLPPSREEMTKNQVSLTCLVKGFYPSDIAVEWE |
| | | | | SNGQPENNYKTTPPVLDSDGSFFLYSKLTVDKS |
| | | | | RWQQGNVFSCSVMHEALHNHYTQKSLSLSPG |
| | | | | KGGGGSGGGGSGGGGSGGGGSGGGGSGGGGS |
| | | | | DKTHTCPPCPAPELLGGPSVFLFPPKPKDTLMIS |
| | | | | RTPEVTCVVVDVSHEDPEVKFNWYVDGVEVH |
| | | | | NAKTKPCEEQYGSTYRCVSVLTVLHQDWLNG |
| | | | | KEYKCKVSNKALPAPIEKTISKAKGQPREPQVY |
| | | | | TLPPSREEMTKNQVSLTCLVKGFYPSDIAVEWE |
| | | | | SNGQPENNYKTTPPVLDSDGSFFLYSKLTVDKS |
| | | | | RWQQGNVFSCSVMHEALHNHYTQKSLSLSPG |
| | | | | KGGGGSGGGGSGGGGSQVQLVQSGAEVKKPG |
| | | | | ASVKVSCKASGYTFTGYYIHWVRQAPGQCLE |
| | | | | WMGWINPNSGGTNYAQKFQGRVTMTRDTSIS |
| | | | | TAYMELSRLRSDDTAVYYCARVEAVAGREYY |
| | | | | YFSGMDVWGQGTTVTVSSGGGGSGGGGSGGG |
| | | | | GSSYELTQPPSVSVSPGQTASITCSGEKLGDKY |
| | | | | VYWYQQKPGQSPVLVIYQSTKRPSGVPERFSG |
| | | | | SNSGNTATLTISGTQAMDEADYYCQAYHASTA |
| | | | | VFGCGTKLTVLSGGGGSEVQLVESGGGLVQPG |
| | | | | GSLKLSCAASGFTFNKYAMNWVRQAPGKGME |
| | | | | WVARIRSKYNNYATYYADAVKDRFTISRDDSK |
| | | | | NTLYLQMNNLKTEDTAVYYCVRAGNFGSSYIS |
| | | | | YFAYWGQGTLVTVSSGGGGSGGGGSGGGGSQ |
| | | | | TVVTQEPSLTVSPGGTVTITCGSSTGAVTSGNY |
| | | | | PNWIQKKPGQAPRGLIGGTKFLAPGTPARFSGS |
| | | | | LEGGKAALTLSGVQPEDEAEYYCVLYYSNRW |
| | | | | VFGSGTKLTVL |
| 270. | CH3 R164L CCx | artificial | Aa | EVQLLESGGGLVQPGGSVRLSCAASGFTFSSY |
| | I2Ccc(44/100)x | | | WMYWVRQAPGKCLEWVSKIDPSDDYTNYNQ |
| | (G4)x scFc x (G4) | | | KVKGRFTISIDNSKNTLYLQMNSLRAEDSAVY |
| | x MS R4L CCx | | | YCARWDYTHFDVWGQGTTVTVSSGGGGSGG |
| | I2Ccc(44/100) - | | | GGSGGGGSEIVMTQSPATLSVSPGERATLSCRA |
| | Full Sequence | | | SSSVSYMHWYQQKPGQAPRLLIYGTSNLASGV |
| | | | | PVRFSGSGSGTEFTLTISRLQSEDVAVYYCQQW |
| | | | | SSYPLTFGCGTKVEIKSGGGGSEVQLVESGGGL |
| | | | | VQPGGSLKLSCAASGFTFNKYAMNWVRQAPG |
| | | | | KCLEWVARIRSKYNNYATYYADSVKDRFTISR |
| | | | | DDSKNTAYLQMNNLKTEDTAVYYCVRHGNFG |
| | | | | NSYISYWAYWGQGTLVTVSSGGGGSGGGGSG |
| | | | | GGGSQTVVTQEPSLTVSPGGTVTLTCGSSTGA |
| | | | | VTSGNYPNWVQQKPGQAPRGLIGGTKFLAPGT |
| | | | | PARFSGSLLGGKAALTLSGVQPEDEAEYYCVL |
| | | | | WYSNRWVFGCGTKLTVLGGGGDKTHTCPPCP |
| | | | | APELLGGPSVFLFPPKPKDTLMISRTPEVTCVV |
| | | | | VDVSHEDPEVKFNWYVDGVEVHNAKTKPCEE |
| | | | | QYGSTYRCVSVLTVLHQDWLNGKEYKCKVSN |
| | | | | KALPAPIEKTISKAKGQPREPQVYTLPPSREEM |
| | | | | TKNQVSLTCLVKGFYPSDIAVEWESNGQPENN |
| | | | | YKTTPPVLDSDGSFFLYSKLTVDKSRWQQGNV |
| | | | | FSCSVMHEALHNHYTQKSLSLSPGKGGGGSGG |
| | | | | GGSGGGGSGGGGSGGGGSGGGGSDKTHTCPP |
| | | | | CPAPELLGGPSVFLFPPKPKDTLMISRTPEVTCV |
| | | | | VVDVSHEDPEVKFNWYVDGVEVHNAKTKPCE |
| | | | | EQYGSTYRCVSVLTVLHQDWLNGKEYKCKVS |
| | | | | NKALPAPIEKTISKAKGQPREPQVYTLPPSREE |
| | | | | MTKNQVSLTCLVKGFYPSDIAVEWESNGQPEN |
| | | | | NYKTTPPVLDSDGSFFLYSKLTVDKSRWQQGN |
| | | | | VFSCSVMHEALHNHYTQKSLSLSPGKGGGGQ |
| | | | | VQLVQSGAEVKKPGASVKVSCKASGYTFTGY |
| | | | | YIHWVRQAPGQCLEWMGWINPNSGGTNYAQ |
| | | | | KFQGRVTMTRDTSISTAYMELSRLRSDDTAVY |
| | | | | YCARVEAVAGREYYYFSGMDVWGQGTTVTV |
| | | | | SSGGGGSGGGGSGGGGSSYELTQPPSVSVSPG |
| | | | | QTASITCSGEKLGDKYVYWYQQKPGQSPVLVI |
| | | | | YQSTKRPSGVPERFSGSNSGNTATLTISGTQAM |
| | | | | DEADYYCQAYHASTAVFGCGTKLTVLSGGGG |
| | | | | SEVQLVESGGGLVQPGGSLKLSCAASGFTFNK |
| | | | | YAMNWVRQAPGKCLEWVARIRSKYNNYATY |
| | | | | YADSVKDRFTISRDDSKNTAYLQMNNLKTEDT |
| | | | | AVYYCVRHGNFGNSYISYWAYWGQGTLVTVS |
| | | | | SGGGGSGGGGSGGGGSQTVVTQEPSLTVSPGG |
| | | | | TVTLTCGSSTGAVTSGNYPNWVQQKPGQAPR |
| | | | | GLIGGTKFLAPGTPARFSGSLLGGKAALTLSGV |
| | | | | QPEDEAEYYCVLWYSNRWVFGCGTKLTVL |
| 271. | CH3 R170R CC x | artificial | Aa | EVQLLESGGGLVQPGGSLRLSCAASGFTFSSY |
| | I2C 44/100cc x | | | WMHWVRQTPGKCLEWVSKIDPSDDYTNYNQ |
| | scFc x MS R4L | | | KVKGRFTISIDKSKNTLYLQMNSLRAEDTAVY |
| | CC x I2C | | | YCARWDYSHFDVWGQGTTVTVSSGGGGSGG |
| | 44/100cc0 - Full | | | GGSGGGGSEIVMTQSPATLSVSPGERATLTCRA |
| | Sequence | | | SSSVSYMHWYQQKPGQAPRLLIYGTSNLVSGV |
| | | | | PARFSGSGSGTEFTLTISSLQSEDFAVYYCQQW |
| | | | | SSYPLTFGCGTKVEIKSGGGGSEVQLVESGGGL |
| | | | | VQPGGSLKLSCAASGFTFNKYAMNWVRQAPG |
| | | | | KCLEWVARIRSKYNNYATYYADSVKDRFTISR |
| | | | | DDSKNTAYLQMNNLKTEDTAVYYCVRHGNFG |
| | | | | NSYISYWAYWGQGTLVTVSSGGGGSGGGGSG |
| | | | | GGGSQTVVTQEPSLTVSPGGTVTLTCGSSTGA |
| | | | | VTSGNYPNWVQQKPGQAPRGLIGGTKFLAPGT |
| | | | | PARFSGSLLGGKAALTLSGVQPEDEAEYYCVL |
| | | | | WYSNRWVFGCGTKLTVLGGGGSGGGGSGGG |
| | | | | GSDKTHTCPPCPAPELLGGPSVFLFPPKPKDTL |
| | | | | MISRTPEVTCVVVDVSHEDPEVKFNWYVDGV |
| | | | | EVHNAKTKPCEEQYGSTYRCVSVLTVLHQDW |
| | | | | LNGKEYKCKVSNKALPAPIEKTISKAKGQPREP |
| | | | | QVYTLPPSREEMTKNQVSLTCLVKGFYPSDIAV |
| | | | | EWESNGQPENNYKTTPPVLDSDGSFFLYSKLT |
| | | | | VDKSRWQQGNVFSCSVMHEALHNHYTQKSLS |
| | | | | LSPGKGGGGSGGGGSGGGGSGGGGSGGGGSG |
| | | | | GGGSDKTHTCPPCPAPELLGGPSVFLFPPKPKD |
| | | | | TLMISRTPEVTCVVVDVSHEDPEVKFNWYVDG |
| | | | | VEVHNAKTKPCEEQYGSTYRCVSVLTVLHQD |
| | | | | WLNGKEYKCKVSNKALPAPIEKTISKAKGQPR |
| | | | | EPQVYTLPPSREEMTKNQVSLTCLVKGFYPSDI |
| | | | | AVEWESNGQPENNYKTTPPVLDSDGSFFLYSK |
| | | | | LTVDKSRWQQGNVFSCSVMHEALHNHYTQKS |
| | | | | LSLSPGKGGGGSGGGGSGGGGSQVQLVQSGA |
| | | | | EVKKPGASVKVSCKASGYTFTGYYIHWVRQA |
| | | | | PGQCLEWMGWINPNSGGTNYAQKFQGRVTMT |
| | | | | RDTSISTAYMELSRLRSDDTAVYYCARVEAVA |
| | | | | GREYYYFSGMDVWGQGTTVTVSSGGGGSGGG |
| | | | | GSGGGGSSYELTQPPSVSVSPGQTASITCSGEK |
| | | | | LGDKYVYWYQQKPGQSPVLVIYQSTKRPSGVP |
| | | | | ERFSGSNSGNTATLTISGTQAMDEADYYCQAY |
| | | | | HASTAVFGCGTKLTVLSGGGGSEVQLVESGGG |
| | | | | LVQPGGSLKLSCAASGFTFNKYAMNWVRQAP |
| | | | | GKCLEWVARIRSKYNNYATYYADSVKDRFTIS |
| | | | | RDDSKNTAYLQMNNLKTEDTAVYYCVRHGNF |
| | | | | GNSYISYWAYWGQGTLVTVSSGGGGSGGGGS |
| | | | | GGGGSQTVVTQEPSLTVSPGGTVTLTCGSSTG |
| | | | | AVTSGNYPNWVQQKPGQAPRGLIGGTKFLAPG |
| | | | | TPARFSGSLLGGKAALTLSGVQPEDEAEYYCV |
| | | | | LWYSNRWVFGCGTKLTVL |
| 272. | MS 01-G11 CCx | artificial | Aa | QVQLVESGGGLVKPGGSLRLSCAASGFTFSDY |
| | 6H10.09x | | | YMTWIRQAPGKCLEWLSYISSSGSTIYYAEAV |
| | (G4S)3x scFcx | | | KGRFTISRDNAKNSLFLQMNSLRAEDTAVYYC |
| | (G4S)3x CH3 | | | ARDRNSHFDYWGQGTLVTVSSGGGGSGGGGS |
| | 005-D5 CCx | | | GGGGSDIMTQSPSSVSASVGDRVTITCRASQGI |
| | 6H10.09 - Full | | | RTWLAWYQQKPGKAPKLLIYGASGLQSGVPS |
| | Sequence | | | RFSGSGSGTDFTLTISSLQPEDFATYYCQQAESF |
| | | | | PRTFGCGTKVEIKSGGGGSEVQLVESGGGLVQ |
| | | | | PGGSLKLSCAASGFTFNKYAMNWVRQAPGKG |
| | | | | MEWVARIRSKYNNYATYYADAVKDRFTISRD |
| | | | | DSKNTLYLQMNNLKTEDTAVYYCVRAGNFGS |
| | | | | SYISYFAYWGQGTLVTVSSGGGGSGGGGSGGG |
| | | | | GSQTVVTQEPSLTVSPGGTVTITCGSSTGAVTS |
| | | | | GNYPNWIQKKPGQAPRGLIGGTKFLAPGTPAR |
| | | | | FSGSLEGGKAALTLSGVQPEDEAEYYCVLYYS |
| | | | | NRWVFGSGTKLTVLGGGGSGGGGSGGGGSDK |
| | | | | THTCPPCPAPELLGGPSVFLFPPKPKDTLMISRT |
| | | | | PEVTCVVVDVSHEDPEVKFNWYVDGVEVHNA |
| | | | | KTKPCEEQYGSTYRCVSVLTVLHQDWLNGKE |
| | | | | YKCKVSNKALPAPIEKTISKAKGQPREPQVYTL |
| | | | | PPSREEMTKNQVSLTCLVKGFYPSDIAVEWES |
| | | | | NGQPENNYKTTPPVLDSDGSFFLYSKLTVDKS |
| | | | | RWQQGNVFSCSVMHEALHNHYTQKSLSLSPG |
| | | | | KGGGGSGGGGSGGGGSGGGGSGGGGSGGGGS |
| | | | | DKTHTCPPCPAPELLGGPSVFLFPPKPKDTLMIS |
| | | | | RTPEVTCVVVDVSHEDPEVKFNWYVDGVEVH |
| | | | | NAKTKPCEEQYGSTYRCVSVLTVLHQDWLNG |
| | | | | KEYKCKVSNKALPAPIEKTISKAKGQPREPQVY |
| | | | | TLPPSREEMTKNQVSLTCLVKGFYPSDIAVEWE |
| | | | | SNGQPENNYKTTPPVLDSDGSFFLYSKLTVDKS |
| | | | | RWQQGNVFSCSVMHEALHNHYTQKSLSLSPG |
| | | | | KGGGGSGGGGSGGGGSEVQLLESGGGLVQPG |
| | | | | GSLRLSCAASGFSFSSYPINWVRQAPGKCLEW |
| | | | | VGVIWTGGGTNYASSVKGRFTISRDNSKNTVY |
| | | | | LQMNSLRAEDTAVYYCAKSRGVYDFKGRGA |
| | | | | MDYWGQGTLVTVSSGGGGSGGGGSGGGGSDI |
| | | | | VMTQSPDSLAVSLGERATINCKSSQSLLYSSNQ |
| | | | | KNYFAWYQQKPGQPPKLLIYWASTRESGVPDR |
| | | | | FSGSGSGTDFTLTISSLQAEDVAVYYCQQYYSY |
| | | | | PYTFGCGTKLEIKSGGGGSEVQLVESGGGLVQ |
| | | | | PGGSLKLSCAASGFTFNKYAMNWVRQAPGKG |
| | | | | MEWVARIRSKYNNYATYYADAVKDRFTISRD |
| | | | | DSKNTLYLQMNNLKTEDTAVYYCVRAGNFGS |
| | | | | SYISYFAYWGQGTLVTVSSGGGGSGGGGSGGG |
| | | | | GSQTVVTQEPSLTVSPGGTVTITCGSSTGAVTS |
| | | | | GNYPNWIQKKPGQAPRGLIGGTKFLAPGTPAR |
| | | | | FSGSLEGGKAALTLSGVQPEDEAEYYCVLYYS |
| | | | | NRWVFGSGTKLTVL |
| 273. | MS 15-B12 CC x | artificial | Aa | QVQLQESGPGLVKPSETLSLTCTVSGGSISSSSY |
| | I2L x (G4Q)3 x | | | FWGWIRQPPGKCLEWIGNIYYSGSSNYNPSLKS |
| | scFc x (G4Q)3 x | | | RVTISVDTSKNQFSLKLSSVTAADTAVYYCAR |
| | CH3 22- A12 CC | | | LPRGDRDAFDIWGQGTMVTVSSGGGGQGGGG |
| | x I2L - Full | | | QGGGGQDIVMTQSPSSLSASVGDRVTITCRASQ |
| | Sequence | | | GISNYLAWYQQKPGKVPKLLIYAASTLQSGVP |
| | | | | SRFSGSGSGTDFTLTISSLQPEDFATYYCQQSYS |
| | | | | TPFTFGCGTKVEIKSGGGGQEVQLVESGGGLV |
| | | | | QPGGSLKLSCAASGFTFNKYAMNWVRQAPGK |
| | | | | GMEWVARIRSKYNNYATYYADAVKDRFTISR |
| | | | | DDSKNTLYLQMNNLKTEDTAVYYCVRAGNFG |
| | | | | SSYISYFAYWGQGTLVTVSSGGGGQGGGGQG |
| | | | | GGGQQTVVTQEPSLTVSPGGTVTITCGSSTGAV |
| | | | | TSGNYPNWIQKKPGQAPRGLIGGTKFLAPGTP |
| | | | | ARFSGSLEGGKAALTLSGVQPEDEAEYYCVLY |
| | | | | YSNRWVFGSGTKLTVLGGGGQGGGGQGGGG |
| | | | | QCPPCPAPELLGGPSVFLFPPKPKDTLMISRTPE |
| | | | | VTCVVVDVSHEEPEVKFNWYVDGVEVHNAKT |
| | | | | KPCEEQYGSTYRCVSVLTVLHQDWLNGKEYK |
| | | | | CKVSNKALPAPIEKTISKAKGQPREPQVYTLPP |
| | | | | SREEMTKNQVSLTCLVKGFYPSDIAVEWESNG |
| | | | | QPENNYKTTPPVLDSDGSFFLYSKLTVDKSRW |
| | | | | QQGNVFSCSVMHEALHNHYTQKSLSLSPGKG |
| | | | | GGGQGGGGQGGGGQGGGGQGGGGQGGGGQ |
| | | | | CPPCPAPELLGGPSVFLFPPKPKDTLMISRTPEV |
| | | | | TCVVVDVSHEEPEVKFNWYVDGVEVHNAKTK |
| | | | | PCEEQYGSTYRCVSVLTVLHQDWLNGKEYKC |
| | | | | KVSNKALPAPIEKTISKAKGQPREPQVYTLPPS |
| | | | | REEMTKNQVSLTCLVKGFYPSDIAVEWESNGQ |
| | | | | PENNYKTTPPVLDSDGSFFLYSKLTVDKSRWQ |
| | | | | QGNVFSCSVMHEALHNHYTQKSLSLSPGKGG |
| | | | | GGQGGGGQGGGGQQVQLVQSGAEVVKPGAS |
| | | | | VKVSCKASGYTFTSSWMNWVRQAPGQCLEW |
| | | | | MGRIYTGTGETKYSGKFQGRVTITRDTSASTA |
| | | | | YMELSSLTSEDTAVYYCARQRDYGALYAMDY |
| | | | | WGQGTLVTVSSGGGGQGGGGQGGGGQDIQLT |
| | | | | QSPSFLSASVGDRVTITCRASDDIYSYLAWYQQ |
| | | | | KPGKAPKLLVYNAKTLAEGVPSRFSGSGSGTE |
| | | | | FTLTISSLQPEDFATYYCQNHDRTPFTFGCGTK |
| | | | | VDIKSGGGGQEVQLVESGGGLVQPGGSLKLSC |
| | | | | AASGFTFNKYAMNWVRQAPGKGMEWVARIR |
| | | | | SKYNNYATYYADAVKDRFTISRDDSKNTLYLQ |
| | | | | MNNLKTEDTAVYYCVRAGNFGSSYISYFAYW |
| | | | | GQGTLVTVSSGGGGQGGGGQGGGGQQTVVT |
| | | | | QEPSLTVSPGGTVTITCGSSTGAVTSGNYPNWI |
| | | | | QKKPGQAPRGLIGGTKFLAPGTPARFSGSLEGG |
| | | | | KAALTLSGVQPEDEAEYYCVLYYSNRWVFGS |
| | | | | GTKLTVL |
| 274. | MS 15-B12 CC x | artificial | Aa | QVQLQESGPGLVKPSETLSLTCTVSGGSISSSSY |
| | I2L x (G4Q)3x | | | FWGWIRQPPGKCLEWIGNIYYSGSSNYNPSLKS |
| | scFc x (G4Q)3 x | | | RVTISVDTSKNQFSLKLSSVTAADTAVYYCAR |
| | CH3 15-E11 CC x | | | LPRGDRDAFDIWGQGTMVTVSSGGGGQGGGG |
| | I2L - Full | | | QGGGGQDIVMTQSPSSLSASVGDRVTITCRASQ |
| | Sequence | | | GISNYLAWYQQKPGKVPKLLIYAASTLQSGVP |
| | | | | SRFSGSGSGTDFTLTISSLQPEDFATYYCQQSYS |
| | | | | TPFTFGCGTKVEIKSGGGGQEVQLVESGGGLV |
| | | | | QPGGSLKLSCAASGFTFNKYAMNWVRQAPGK |
| | | | | GMEWVARIRSKYNNYATYYADAVKDRFTISR |
| | | | | DDSKNTLYLQMNNLKTEDTAVYYCVRAGNFG |
| | | | | SSYISYFAYWGQGTLVTVSSGGGGQGGGGQG |
| | | | | GGGQQTVVTQEPSLTVSPGGTVTITCGSSTGAV |
| | | | | TSGNYPNWIQKKPGQAPRGLIGGTKFLAPGTP |
| | | | | ARFSGSLEGGKAALTLSGVQPEDEAEYYCVLY |
| | | | | YSNRWVFGSGTKLTVLGGGGQGGGGQGGGG |
| | | | | QCPPCPAPELLGGPSVFLFPPKPKDTLMISRTPE |
| | | | | VTCVVVDVSHEEPEVKFNWYVDGVEVHNAKT |
| | | | | KPCEEQYGSTYRCVSVLTVLHQDWLNGKEYK |
| | | | | CKVSNKALPAPIEKTISKAKGQPREPQVYTLPP |
| | | | | SREEMTKNQVSLTCLVKGFYPSDIAVEWESNG |
| | | | | QPENNYKTTPPVLDSDGSFFLYSKLTVDKSRW |
| | | | | QQGNVFSCSVMHEALHNHYTQKSLSLSPGKG |
| | | | | GGGQGGGGQGGGGQGGGGQGGGGQGGGGQ |
| | | | | CPPCPAPELLGGPSVFLFPPKPKDTLMISRTPEV |
| | | | | TCVVVDVSHEEPEVKFNWYVDGVEVHNAKTK |
| | | | | PCEEQYGSTYRCVSVLTVLHQDWLNGKEYKC |
| | | | | KVSNKALPAPIEKTISKAKGQPREPQVYTLPPS |
| | | | | REEMTKNQVSLTCLVKGFYPSDIAVEWESNGQ |
| | | | | PENNYKTTPPVLDSDGSFFLYSKLTVDKSRWQ |
| | | | | QGNVFSCSVMHEALHNHYTQKSLSLSPGKGG |
| | | | | GGQGGGGQGGGGQQVQLVQSGAEVKKPGAS |
| | | | | VKVSCKASGYTFTNYWMNWVRQAPGQCLEW |
| | | | | MGNIAYGVKGTNYNQKFQGRVTMTVDTSSST |
| | | | | AYMELSRLRSDDTAVYYCATRYFYVMDYWG |
| | | | | QGTLVTVSSGGGGQGGGGQGGGGQDIQMTQS |
| | | | | PSSLSASVGDRVTITCRASQDISNYLNWYQQKP |
| | | | | GKVPKLLIYYTSRLHSGVPSRFSGSGSGTDFTL |
| | | | | TISSLQPEDVATYYCVQYAQFPLTFGCGTKVEI |
| | | | | KSGGGGQEVQLVESGGGLVQPGGSLKLSCAAS |
| | | | | GFTFNKYAMNWVRQAPGKGMEWVARIRSKY |
| | | | | NNYATYYADAVKDRFTISRDDSKNTLYLQMN |
| | | | | NLKTEDTAVYYCVRAGNFGSSYISYFAYWGQ |
| | | | | GTLVTVSSGGGGQGGGGQGGGGQQTVVTQEP |
| | | | | SLTVSPGGTVTITCGSSTGAVTSGNYPNWIQKK |
| | | | | PGQAPRGLIGGTKFLAPGTPARFSGSLEGGKAA |
| | | | | LTLSGVQPEDEAEYYCVLYYSNRWVFGSGTKL |
| | | | | TVL |
| 275. | MS 15-B12 CC x | artificial | Aa | QVQLQESGPGLVKPSETLSLTCTVSGGSISSSSY |
| | I2L x G4 x scFc | | | FWGWIRQPPGKCLEWIGNIYYSGSSNYNPSLKS |
| | xG4 x CH3 26-E5 | | | RVTISVDTSKNQFSLKLSSVTAADTAVYYCAR |
| | CC x I2L_GQ - | | | LPRGDRDAFDIWGQGTMVTVSSGGGGQGGGG |
| | Full Sequence | | | QGGGGQDIVMTQSPSSLSASVGDRVTITCRASQ |
| | | | | GISNYLAWYQQKPGKVPKLLIYAASTLQSGVP |
| | | | | SRFSGSGSGTDFTLTISSLQPEDFATYYCQQSYS |
| | | | | TPFTFGCGTKVEIKSGGGGQEVQLVESGGGLV |
| | | | | QPGGSLKLSCAASGFTFNKYAMNWVRQAPGK |
| | | | | GMEWVARIRSKYNNYATYYADAVKDRFTISR |
| | | | | DDSKNTLYLQMNNLKTEDTAVYYCVRAGNFG |
| | | | | SSYISYFAYWGQGTLVTVSSGGGGQGGGGQG |
| | | | | GGGQQTVVTQEPSLTVSPGGTVTITCGSSTGAV |
| | | | | TSGNYPNWIQKKPGQAPRGLIGGTKFLAPGTP |
| | | | | ARFSGSLEGGKAALTLSGVQPEDEAEYYCVLY |
| | | | | YSNRWVFGSGTKLTVLGGGGCPPCPAPELLGG |
| | | | | PSVFLFPPKPKDTLMISRTPEVTCVVVDVSHEE |
| | | | | PEVKFNWYVDGVEVHNAKTKPCEEQYGSTYR |
| | | | | CVSVLTVLHQDWLNGKEYKCKVSNKALPAPIE |
| | | | | KTISKAKGQPREPQVYTLPPSREEMTKNQVSLT |
| | | | | CLVKGFYPSDIAVEWESNGQPENNYKTTPPVL |
| | | | | DSDGSFFLYSKLTVDKSRWQQGNVFSCSVMHE |
| | | | | ALHNHYTQKSLSLSPGKGGGGQGGGGQGGGG |
| | | | | QGGGGQGGGGQGGGGQCPPCPAPELLGGPSV |
| | | | | FLFPPKPKDTLMISRTPEVTCVVVDVSHEEPEV |
| | | | | KFNWYVDGVEVHNAKTKPCEEQYGSTYRCVS |
| | | | | VLTVLHQDWLNGKEYKCKVSNKALPAPIEKTI |
| | | | | SKAKGQPREPQVYTLPPSREEMTKNQVSLTCL |
| | | | | VKGFYPSDIAVEWESNGQPENNYKTTPPVLDS |
| | | | | DGSFFLYSKLTVDKSRWQQGNVFSCSVMHEA |
| | | | | LHNHYTQKSLSLSPGKGGGGQVQLVQSGAEV |
| | | | | KKPGASVKVSCKASGYTFTSYWMHWVRQAP |
| | | | | GQCLEWMGVIRTSTSYTIYNQKFKGRVTMTRD |
| | | | | TSTSTVYMELSSLRSEDTAVYYCARSGPGYFD |
| | | | | VWGQGTMVTVSSGGGGQGGGGQGGGGQDIQ |
| | | | | LTQSPSFLSASVGDRVTITCRASENIYSYLAWY |
| | | | | QQKPGKAPKLLIYNAKTLAEGVPSRFSGSGSGT |
| | | | | EFTLTISSLQPEDFATYYCQHNYGTPYTFGCGT |
| | | | | KLEIKSGGGGQEVQLVESGGGLVQPGGSLKLS |
| | | | | CAASGFTFNKYAMNWVRQAPGKGMEWVARI |
| | | | | RSKYNNYATYYADAVKDRFTISRDDSKNTLYL |
| | | | | QMNNLKTEDTAVYYCVRAGNFGSSYISYFAY |
| | | | | WGQGTLVTVSSGGGGQGGGGQGGGGQQTVV |
| | | | | TQEPSLTVSPGGTVTITCGSSTGAVTSGNYPNW |
| | | | | IQKKPGQAPRGLIGGTKFLAPGTPARFSGSLEG |
| | | | | GKAALTLSGVQPEDEAEYYCVLYYSNRWVFG |
| | | | | SGTKLTVL |
| 276. | MS 15-B12 CC x | artificial | Aa | QVQLQESGPGLVKPSETLSLTCTVSGGSISSSSY |
| | I2L x G4 x scFc x | | | FWGWIRQPPGKCLEWIGNIYYSGSSNYNPSLKS |
| | G4 x CH3 005-D5 | | | RVTISVDTSKNQFSLKLSSVTAADTAVYYCAR |
| | CC x I2L_GQ - | | | LPRGDRDAFDIWGQGTMVTVSSGGGGQGGGG |
| | Full Sequence | | | QGGGGQDIVMTQSPSSLSASVGDRVTITCRASQ |
| | | | | GISNYLAWYQQKPGKVPKLLIYAASTLQSGVP |
| | | | | SRFSGSGSGTDFTLTISSLQPEDFATYYCQQSYS |
| | | | | TPFTFGCGTKVEIKSGGGGQEVQLVESGGGLV |
| | | | | QPGGSLKLSCAASGFTFNKYAMNWVRQAPGK |
| | | | | GMEWVARIRSKYNNYATYYADAVKDRFTISR |
| | | | | DDSKNTLYLQMNNLKTEDTAVYYCVRAGNFG |
| | | | | SSYISYFAYWGQGTLVTVSSGGGGQGGGGQG |
| | | | | GGGQQTVVTQEPSLTVSPGGTVTITCGSSTGAV |
| | | | | TSGNYPNWIQKKPGQAPRGLIGGTKFLAPGTP |
| | | | | ARFSGSLEGGKAALTLSGVQPEDEAEYYCVLY |
| | | | | YSNRWVFGSGTKLTVLGGGGCPPCPAPELLGG |
| | | | | PSVFLFPPKPKDTLMISRTPEVTCVVVDVSHEE |
| | | | | PEVKFNWYVDGVEVHNAKTKPCEEQYGSTYR |
| | | | | CVSVLTVLHQDWLNGKEYKCKVSNKALPAPIE |
| | | | | KTISKAKGQPREPQVYTLPPSREEMTKNQVSLT |
| | | | | CLVKGFYPSDIAVEWESNGQPENNYKTTPPVL |
| | | | | DSDGSFFLYSKLTVDKSRWQQGNVFSCSVMHE |
| | | | | ALHNHYTQKSLSLSPGKGGGGQGGGGQGGGG |
| | | | | QGGGGQGGGGQGGGGQCPPCPAPELLGGPSV |
| | | | | FLFPPKPKDTLMISRTPEVTCVVVDVSHEEPEV |
| | | | | KFNWYVDGVEVHNAKTKPCEEQYGSTYRCVS |
| | | | | VLTVLHQDWLNGKEYKCKVSNKALPAPIEKTI |
| | | | | SKAKGQPREPQVYTLPPSREEMTKNQVSLTCL |
| | | | | VKGFYPSDIAVEWESNGQPENNYKTTPPVLDS |
| | | | | DGSFFLYSKLTVDKSRWQQGNVFSCSVMHEA |
| | | | | LHNHYTQKSLSLSPGKGGGGEVQLLESGGGLV |
| | | | | QPGGSLRLSCAASGFSFSSYPINWVRQAPGKCL |
| | | | | EWVGVIWTGGGTNYASSVKGRFTISRDNSKNT |
| | | | | VYLQMNSLRAEDTAVYYCAKSRGVYDFKGRG |
| | | | | AMDYWGQGTLVTVSSGGGGQGGGGQGGGGQ |
| | | | | DIVMTQSPDSLAVSLGERATINCKSSQSLLYSS |
| | | | | NQKNYFAWYQQKPGQPPKLLIYWASTRESGV |
| | | | | PDRFSGSGSGTDFTLTISSLQAEDVAVYYCQQY |
| | | | | YSYPYTFGCGTKLEIKSGGGGQEVQLVESGGG |
| | | | | LVQPGGSLKLSCAASGFTFNKYAMNWVRQAP |
| | | | | GKGMEWVARIRSKYNNYATYYADAVKDRFTI |
| | | | | SRDDSKNTLYLQMNNLKTEDTAVYYCVRAGN |
| | | | | FGSSYISYFAYWGQGTLVTVSSGGGGQGGGGQ |
| | | | | GGGGQQTVVTQEPSLTVSPGGTVTITCGSSTGA |
| | | | | VTSGNYPNWIQKKPGQAPRGLIGGTKFLAPGT |
| | | | | PARFSGSLEGGKAALTLSGVQPEDEAEYYCVL |
| | | | | YYSNRWVFGSGTKLTVL |
| 277. | MS 15-B12 CC x | artificial | Aa | QVQLQESGPGLVKPSETLSLTCTVSGGSISSSSY |
| | I2L x G4 x scFc | | | FWGWIRQPPGKCLEWIGNIYYSGSSNYNPSLKS |
| | xG4 x CH3 15- | | | RVTISVDTSKNQFSLKLSSVTAADTAVYYCAR |
| | E11 CC x I2L | | | LPRGDRDAFDIWGQGTMVTVSSGGGGQGGGG |
| | _GQ - Full | | | QGGGGQDIVMTQSPSSLSASVGDRVTITCRASQ |
| | Sequence | | | GISNYLAWYQQKPGKVPKLLIYAASTLQSGVP |
| | | | | SRFSGSGSGTDFTLTISSLQPEDFATYYCQQSYS |
| | | | | TPFTFGCGTKVEIKSGGGGQEVQLVESGGGLV |
| | | | | QPGGSLKLSCAASGFTFNKYAMNWVRQAPGK |
| | | | | GMEWVARIRSKYNNYATYYADAVKDRFTISR |
| | | | | DDSKNTLYLQMNNLKTEDTAVYYCVRAGNFG |
| | | | | SSYISYFAYWGQGTLVTVSSGGGGQGGGGQG |
| | | | | GGGQQTVVTQEPSLTVSPGGTVTITCGSSTGAV |
| | | | | TSGNYPNWIQKKPGQAPRGLIGGTKFLAPGTP |
| | | | | ARFSGSLEGGKAALTLSGVQPEDEAEYYCVLY |
| | | | | YSNRWVFGSGTKLTVLGGGGCPPCPAPELLGG |
| | | | | PSVFLFPPKPKDTLMISRTPEVTCVVVDVSHEE |
| | | | | PEVKFNWYVDGVEVHNAKTKPCEEQYGSTYR |
| | | | | CVSVLTVLHQDWLNGKEYKCKVSNKALPAPIE |
| | | | | KTISKAKGQPREPQVYTLPPSREEMTKNQVSLT |
| | | | | CLVKGFYPSDIAVEWESNGQPENNYKTTPPVL |
| | | | | DSDGSFFLYSKLTVDKSRWQQGNVFSCSVMHE |
| | | | | ALHNHYTQKSLSLSPGKGGGGQGGGGQGGGG |
| | | | | QGGGGQGGGGQGGGGQCPPCPAPELLGGPSV |
| | | | | FLFPPKPKDTLMISRTPEVTCVVVDVSHEEPEV |
| | | | | KFNWYVDGVEVHNAKTKPCEEQYGSTYRCVS |
| | | | | VLTVLHQDWLNGKEYKCKVSNKALPAPIEKTI |
| | | | | SKAKGQPREPQVYTLPPSREEMTKNQVSLTCL |
| | | | | VKGFYPSDIAVEWESNGQPENNYKTTPPVLDS |
| | | | | DGSFFLYSKLTVDKSRWQQGNVFSCSVMHEA |
| | | | | LHNHYTQKSLSLSPGKGGGGQVQLVQSGAEV |
| | | | | KKPGASVKVSCKASGYTFTNYWMNWVRQAP |
| | | | | GQCLEWMGNIAYGVKGTNYNQKFQGRVTMT |
| | | | | VDTSSSTAYMELSRLRSDDTAVYYCATRYFYV |
| | | | | MDYWGQGTLVTVSSGGGGQGGGGQGGGGQD |
| | | | | IQMTQSPSSLSASVGDRVTITCRASQDISNYLN |
| | | | | WYQQKPGKVPKLLIYYTSRLHSGVPSRFSGSGS |
| | | | | GTDFTLTISSLQPEDVATYYCVQYAQFPLTFGC |
| | | | | GTKVEIKSGGGGQEVQLVESGGGLVQPGGSLK |
| | | | | LSCAASGFTFNKYAMNWVRQAPGKGMEWVA |
| | | | | RIRSKYNNYATYYADAVKDRFTISRDDSKNTL |
| | | | | YLQMNNLKTEDTAVYYCVRAGNFGSSYISYFA |
| | | | | YWGQGTLVTVSSGGGGQGGGGQGGGGQQTV |
| | | | | VTQEPSLTVSPGGTVTITCGSSTGAVTSGNYPN |
| | | | | WIQKKPGQAPRGLIGGTKFLAPGTPARFSGSLE |
| | | | | GGKAALTLSGVQPEDEAEYYCVLYYSNRWVF |
| | | | | GSGTKLTVL |
| 278. | MS 15-B12 CC x | artificial | Aa | QVQLQESGPGLVKPSETLSLTCTVSGGSISSSSY |
| | I2L x G4S3 x scFc | | | FWGWIRQPPGKCLEWIGNIYYSGSSNYNPSLKS |
| | x G4S3 x CH3 26- | | | RVTISVDTSKNQFSLKLSSVTAADTAVYYCAR |
| | E5 CC x I2L - Full | | | LPRGDRDAFDIWGQGTMVTVSSGGGGSGGGG |
| | Sequence | | | SGGGGSDIVMTQSPSSLSASVGDRVTITCRASQ |
| | | | | GISNYLAWYQQKPGKVPKLLIYAASTLQSGVP |
| | | | | SRFSGSGSGTDFTLTISSLQPEDFATYYCQQSYS |
| | | | | TPFTFGCGTKVEIKSGGGGSEVQLVESGGGLV |
| | | | | QPGGSLKLSCAASGFTFNKYAMNWVRQAPGK |
| | | | | GMEWVARIRSKYNNYATYYADAVKDRFTISR |
| | | | | DDSKNTLYLQMNNLKTEDTAVYYCVRAGNFG |
| | | | | SSYISYFAYWGQGTLVTVSSGGGGSGGGGSGG |
| | | | | GGSQTVVTQEPSLTVSPGGTVTITCGSSTGAVT |
| | | | | SGNYPNWIQKKPGQAPRGLIGGTKFLAPGTPA |
| | | | | RFSGSLEGGKAALTLSGVQPEDEAEYYCVLYY |
| | | | | SNRWVFGSGTKLTVLGGGGSGGGGSGGGGSD |
| | | | | KTHTCPPCPAPELLGGPSVFLFPPKPKDTLMISR |
| | | | | TPEVTCVVVDVSHEDPEVKFNWYVDGVEVHN |
| | | | | AKTKPCEEQYGSTYRCVSVLTVLHQDWLNGK |
| | | | | EYKCKVSNKALPAPIEKTISKAKGQPREPQVYT |
| | | | | LPPSREEMTKNQVSLTCLVKGFYPSDIAVEWES |
| | | | | NGQPENNYKTTPPVLDSDGSFFLYSKLTVDKS |
| | | | | RWQQGNVFSCSVMHEALHNHYTQKSLSLSPG |
| | | | | KGGGGSGGGGSGGGGSGGGGSGGGGSGGGGS |
| | | | | DKTHTCPPCPAPELLGGPSVFLFPPKPKDTLMIS |
| | | | | RTPEVTCVVVDVSHEDPEVKFNWYVDGVEVH |
| | | | | NAKTKPCEEQYGSTYRCVSVLTVLHQDWLNG |
| | | | | KEYKCKVSNKALPAPIEKTISKAKGQPREPQVY |
| | | | | TLPPSREEMTKNQVSLTCLVKGFYPSDIAVEWE |
| | | | | SNGQPENNYKTTPPVLDSDGSFFLYSKLTVDKS |
| | | | | RWQQGNVFSCSVMHEALHNHYTQKSLSLSPG |
| | | | | KGGGGSGGGGSGGGGSQVQLVQSGAEVKKPG |
| | | | | ASVKVSCKASGYTFTSYWMHWVRQAPGQCLE |
| | | | | WMGVIRTSTSYTIYNQKFKGRVTMTRDTSTST |
| | | | | VYMELSSLRSEDTAVYYCARSGPGYFDVWGQ |
| | | | | GTMVTVSSGGGGSGGGGSGGGGSDIQLTQSPS |
| | | | | FLSASVGDRVTITCRASENIYSYLAWYQQKPG |
| | | | | KAPKLLIYNAKTLAEGVPSRFSGSGSGTEFTLTI |
| | | | | SSLQPEDFATYYCQHNYGTPYTFGCGTKLEIKS |
| | | | | GGGGSEVQLVESGGGLVQPGGSLKLSCAASGF |
| | | | | TFNKYAMNWVRQAPGKGMEWVARIRSKYNN |
| | | | | YATYYADAVKDRFTISRDDSKNTLYLQMNNL |
| | | | | KTEDTAVYYCVRAGNFGSSYISYFAYWGQGTL |
| | | | | VTVSSGGGGSGGGGSGGGGSQTVVTQEPSLTV |
| | | | | SPGGTVTITCGSSTGAVTSGNYPNWIQKKPGQ |
| | | | | APRGLIGGTKFLAPGTPARFSGSLEGGKAALTL |
| | | | | SGVQPEDEAEYYCVLYYSNRWVFGSGTKLTV |
| | | | | L |
| 279. | MS 15-B12 CC x | artificial | aa | QVQLQESGPGLVKPSETLSLTCTVSGGSISSSSY |
| | I2Lx G4S3 x scFc | | | FWGWIRQPPGKCLEWIGNIYYSGSSNYNPSLKS |
| | x G4S3 x CH3 24- | | | RVTISVDTSKNQFSLKLSSVTAADTAVYYCAR |
| | D7 CC x I2L - | | | LPRGDRDAFDIWGQGTMVTVSSGGGGSGGGG |
| | Full Sequence | | | SGGGGSDIVMTQSPSSLSASVGDRVTITCRASQ |
| | | | | GISNYLAWYQQKPGKVPKLLIYAASTLQSGVP |
| | | | | SRFSGSGSGTDFTLTISSLQPEDFATYYCQQSYS |
| | | | | TPFTFGCGTKVEIKSGGGGSEVQLVESGGGLV |
| | | | | QPGGSLKLSCAASGFTFNKYAMNWVRQAPGK |
| | | | | GMEWVARIRSKYNNYATYYADAVKDRFTISR |
| | | | | DDSKNTLYLQMNNLKTEDTAVYYCVRAGNFG |
| | | | | SSYISYFAYWGQGTLVTVSSGGGGSGGGGSGG |
| | | | | GGSQTVVTQEPSLTVSPGGTVTITCGSSTGAVT |
| | | | | SGNYPNWIQKKPGQAPRGLIGGTKFLAPGTPA |
| | | | | RFSGSLEGGKAALTLSGVQPEDEAEYYCVLYY |
| | | | | SNRWVFGSGTKLTVLGGGGSGGGGSGGGGSD |
| | | | | KTHTCPPCPAPELLGGPSVFLFPPKPKDTLMISR |
| | | | | TPEVTCVVVDVSHEDPEVKFNWYVDGVEVHN |
| | | | | AKTKPCEEQYGSTYRCVSVLTVLHQDWLNGK |
| | | | | EYKCKVSNKALPAPIEKTISKAKGQPREPQVYT |
| | | | | LPPSREEMTKNQVSLTCLVKGFYPSDIAVEWES |
| | | | | NGQPENNYKTTPPVLDSDGSFFLYSKLTVDKS |
| | | | | RWQQGNVFSCSVMHEALHNHYTQKSLSLSPG |
| | | | | KGGGGSGGGGSGGGGSGGGGSGGGGSGGGGS |
| | | | | DKTHTCPPCPAPELLGGPSVFLFPPKPKDTLMIS |
| | | | | RTPEVTCVVVDVSHEDPEVKFNWYVDGVEVH |
| | | | | NAKTKPCEEQYGSTYRCVSVLTVLHQDWLNG |
| | | | | KEYKCKVSNKALPAPIEKTISKAKGQPREPQVY |
| | | | | TLPPSREEMTKNQVSLTCLVKGFYPSDIAVEWE |
| | | | | SNGQPENNYKTTPPVLDSDGSFFLYSKLTVDKS |
| | | | | RWQQGNVFSCSVMHEALHNHYTQKSLSLSPG |
| | | | | KGGGGSGGGGSGGGGSQVQLVQSGAEVKKPG |
| | | | | ASVKVSCKASGYTFTNYWMNWVRQAPGQCL |
| | | | | EWMGNIHSKAHGTNYNQKFQGRVTMTVDTSS |
| | | | | STAYMELSRLRSDDTAVYYCATRYFYVMDYW |
| | | | | GQGTLVTVSSGGGGSGGGGSGGGGSDIQMTQS |
| | | | | PSSLSASVGDRVTITCRASQDISNYLNWYQQKP |
| | | | | GKVPKLLIYYTSRLHSGVPSRFSGSGSGTDFTL |
| | | | | TISSLQPEDVATYYCVQYAQFPLTFGCGTKVEI |
| | | | | KSGGGGSEVQLVESGGGLVQPGGSLKLSCAAS |
| | | | | GFTFNKYAMNWVRQAPGKGMEWVARIRSKY |
| | | | | NNYATYYADAVKDRFTISRDDSKNTLYLQMN |
| | | | | NLKTEDTAVYYCVRAGNFGSSYISYFAYWGQ |
| | | | | GTLVTVSSGGGGSGGGGSGGGGSQTVVTQEPS |
| | | | | LTVSPGGTVTITCGSSTGAVTSGNYPNWIQKKP |
| | | | | GQAPRGLIGGTKFLAPGTPARFSGSLEGGKAAL |
| | | | | TLSGVQPEDEAEYYCVLYYSNRWVFGSGTKLT |
| | | | | VL |
| 280. | MS 15-B12 CC x | artificial | aa | QVQLQESGPGLVKPSETLSLTCTVSGGSISSSSY |
| | I2M2 x G4 x scfc | | | FWGWIRQPPGKCLEWIGNIYYSGSSNYNPSLKS |
| | x G4 x CH3 15- | | | RVTISVDTSKNQFSLKLSSVTAADTAVYYCAR |
| | E11 CC xI2M2 - | | | LPRGDRDAFDIWGQGTMVTVSSGGGGSGGGG |
| | Full Sequence | | | SGGGGSDIVMTQSPSSLSASVGDRVTITCRASQ |
| | | | | GISNYLAWYQQKPGKVPKLLIYAASTLQSGVP |
| | | | | SRFSGSGSGTDFTLTISSLQPEDFATYYCQQSYS |
| | | | | TPFTFGCGTKVEIKSGGGGSEVQLVESGGGLV |
| | | | | QPGGSLKLSCAASGFTFNKYAINWVREAPGKG |
| | | | | LEWVARIRSKYNNYATYYADAVKDRFTISRDD |
| | | | | SKNTAYLQMNNLKTEDTAVYYCVRNANFGTS |
| | | | | YISYFAYWGQGTLVTVSSGGGGSGGGGSGGG |
| | | | | GSQTVVTQEPSLTVSPGGTVTLTCGSSTGAVTS |
| | | | | GNYPNWVQKKPGQAPRGLIGGTKFLAPGTPAR |
| | | | | FSGSLLGGKAALTLSGVQPEDEAEYYCVLWYS |
| | | | | NRWVFGSGTKLTVLGGGGDKTHTCPPCPAPEL |
| | | | | LGGPSVFLFPPKPKDTLMISRTPEVTCVVVDVS |
| | | | | HEDPEVKFNWYVDGVEVHNAKTKPCEEQYGS |
| | | | | TYRCVSVLTVLHQDWLNGKEYKCKVSNKALP |
| | | | | APIEKTISKAKGQPREPQVYTLPPSREEMTKNQ |
| | | | | VSLTCLVKGFYPSDIAVEWESNGQPENNYKTT |
| | | | | PPVLDSDGSFFLYSKLTVDKSRWQQGNVFSCS |
| | | | | VMHEALHNHYTQKSLSLSPGKGGGGSGGGGS |
| | | | | GGGGSGGGGSGGGGSGGGGSDKTHTCPPCPAP |
| | | | | ELLGGPSVFLFPPKPKDTLMISRTPEVTCVVVD |
| | | | | VSHEDPEVKFNWYVDGVEVHNAKTKPCEEQY |
| | | | | GSTYRCVSVLTVLHQDWLNGKEYKCKVSNKA |
| | | | | LPAPIEKTISKAKGQPREPQVYTLPPSREEMTK |
| | | | | NQVSLTCLVKGFYPSDIAVEWESNGQPENNYK |
| | | | | TTPPVLDSDGSFFLYSKLTVDKSRWQQGNVFS |
| | | | | CSVMHEALHNHYTQKSLSLSPGKGGGGQVQL |
| | | | | VQSGAEVKKPGASVKVSCKASGYTFTNYWMN |
| | | | | WVRQAPGQCLEWMGNIAYGVKGTNYNQKFQ |
| | | | | GRVTMTVDTSSSTAYMELSRLRSDDTAVYYC |
| | | | | ATRYFYVMDYWGQGTLVTVSSGGGGSGGGGS |
| | | | | GGGGSDIQMTQSPSSLSASVGDRVTITCRASQD |
| | | | | ISNYLNWYQQKPGKVPKLLIYYTSRLHSGVPSR |
| | | | | FSGSGSGTDFTLTISSLQPEDVATYYCVQYAQF |
| | | | | PLTFGCGTKVEIKSGGGGSEVQLVESGGGLVQ |
| | | | | PGGSLKLSCAASGFTFNKYAINWVREAPGKGL |
| | | | | EWVARIRSKYNNYATYYADAVKDRFTISRDDS |
| | | | | KNTAYLQMNNLKTEDTAVYYCVRNANFGTSY |
| | | | | ISYFAYWGQGTLVTVSSGGGGSGGGGSGGGGS |
| | | | | QTVVTQEPSLTVSPGGTVTLTCGSSTGAVTSGN |
| | | | | YPNWVQKKPGQAPRGLIGGTKFLAPGTPARFS |
| | | | | GSLLGGKAALTLSGVQPEDEAEYYCVLWYSN |
| | | | | RWVFGSGTKLTVL |
| 281. | MS 15-B12 CC x | artificial | aa | QVQLQESGPGLVKPSETLSLTCTVSGGSISSSSY |
| | I2M2 x (G4Q)3x | | | FWGWIRQPPGKCLEWIGNIYYSGSSNYNPSLKS |
| | scFcmod x | | | RVTISVDTSKNQFSLKLSSVTAADTAVYYCAR |
| | (G4Q)3 x CH3 22- | | | LPRGDRDAFDIWGQGTMVTVSSGGGGQGGGG |
| | A12 CC x | | | QGGGGQDIVMTQSPSSLSASVGDRVTITCRASQ |
| | I2M2_GQ - Full | | | GISNYLAWYQQKPGKVPKLLIYAASTLQSGVP |
| | Sequence | | | SRFSGSGSGTDFTLTISSLQPEDFATYYCQQSYS |
| | | | | TPFTFGCGTKVEIKSGGGGQEVQLVESGGGLV |
| | | | | QPGGSLKLSCAASGFTFNKYAINWVREAPGKG |
| | | | | LEWVARIRSKYNNYATYYADAVKDRFTISRDD |
| | | | | SKNTAYLQMNNLKTEDTAVYYCVRNANFGTS |
| | | | | YISYFAYWGQGTLVTVSSGGGGQGGGGQGGG |
| | | | | GQQTVVTQEPSLTVSPGGTVTLTCGSSTGAVTS |
| | | | | GNYPNWVQKKPGQAPRGLIGGTKFLAPGTPAR |
| | | | | FSGSLLGGKAALTLSGVQPEDEAEYYCVLWYS |
| | | | | NRWVFGSGTKLTVLGGGGQGGGGQGGGGQC |
| | | | | PPCPAPELLGGPSVFLFPPKPKDTLMISRTPEVT |
| | | | | CVVVDVSHEEPEVKFNWYVDGVEVHNAKTKP |
| | | | | CEEQYGSTYRCVSVLTVLHQDWLNGKEYKCK |
| | | | | VSNKALPAPIEKTISKAKGQPREPQVYTLPPSRE |
| | | | | EMTKNQVSLTCLVKGFYPSDIAVEWESNGQPE |
| | | | | NNYKTTPPVLDSDGSFFLYSKLTVDKSRWQQG |
| | | | | NVFSCSVMHEALHNHYTQKSLSLSPGKGGGG |
| | | | | QGGGGQGGGGQGGGGQGGGGQGGGGQCPPC |
| | | | | PAPELLGGPSVFLFPPKPKDTLMISRTPEVTCVV |
| | | | | VDVSHEEPEVKFNWYVDGVEVHNAKTKPCEE |
| | | | | QYGSTYRCVSVLTVLHQDWLNGKEYKCKVSN |
| | | | | KALPAPIEKTISKAKGQPREPQVYTLPPSREEM |
| | | | | TKNQVSLTCLVKGFYPSDIAVEWESNGQPENN |
| | | | | YKTTPPVLDSDGSFFLYSKLTVDKSRWQQGNV |
| | | | | FSCSVMHEALHNHYTQKSLSLSPGKGGGGQG |
| | | | | GGGQGGGGQQVQLVQSGAEVVKPGASVKVSC |
| | | | | KASGYTFTSSWMNWVRQAPGQCLEWMGRIYT |
| | | | | GTGETKYSGKFQGRVTITRDTSASTAYMELSSL |
| | | | | TSEDTAVYYCARQRDYGALYAMDYWGQGTL |
| | | | | VTVSSGGGGQGGGGQGGGGQDIQLTQSPSFLS |
| | | | | ASVGDRVTITCRASDDIYSYLAWYQQKPGKAP |
| | | | | KLLVYNAKTLAEGVPSRFSGSGSGTEFTLTISSL |
| | | | | QPEDFATYYCQNHDRTPFTFGCGTKVDIKSGG |
| | | | | GGQEVQLVESGGGLVQPGGSLKLSCAASGFTF |
| | | | | NKYAINWVREAPGKGLEWVARIRSKYNNYAT |
| | | | | YYADAVKDRFTISRDDSKNTAYLQMNNLKTE |
| | | | | DTAVYYCVRNANFGTSYISYFAYWGQGTLVT |
| | | | | VSSGGGGQGGGGQGGGGQQTVVTQEPSLTVS |
| | | | | PGGTVTLTCGSSTGAVTSGNYPNWVQKKPGQ |
| | | | | APRGLIGGTKFLAPGTPARFSGSLLGGKAALTL |
| | | | | SGVQPEDEAEYYCVLWYSNRWVFGSGTKLTV |
| | | | | L |
| 282. | MS 15-B12 CC x | artificial | aa | QVQLQESGPGLVKPSETLSLTCTVSGGSISSSSY |
| | I2M2 x (G4Q)3x | | | FWGWIRQPPGKCLEWIGNIYYSGSSNYNPSLKS |
| | scFcmod x | | | RVTISVDTSKNQFSLKLSSVTAADTAVYYCAR |
| | (G4Q3) x CH3 15- | | | LPRGDRDAFDIWGQGTMVTVSSGGGGQGGGG |
| | E11 CC x I2M2 - | | | QGGGGQDIVMTQSPSSLSASVGDRVTITCRASQ |
| | Full Sequence | | | GISNYLAWYQQKPGKVPKLLIYAASTLQSGVP |
| | | | | SRFSGSGSGTDFTLTISSLQPEDFATYYCQQSYS |
| | | | | TPFTFGCGTKVEIKSGGGGQEVQLVESGGGLV |
| | | | | QPGGSLKLSCAASGFTFNKYAINWVREAPGKG |
| | | | | LEWVARIRSKYNNYATYYADAVKDRFTISRDD |
| | | | | SKNTAYLQMNNLKTEDTAVYYCVRNANFGTS |
| | | | | YISYFAYWGQGTLVTVSSGGGGQGGGGQGGG |
| | | | | GQQTVVTQEPSLTVSPGGTVTLTCGSSTGAVTS |
| | | | | GNYPNWVQKKPGQAPRGLIGGTKFLAPGTPAR |
| | | | | FSGSLLGGKAALTLSGVQPEDEAEYYCVLWYS |
| | | | | NRWVFGSGTKLTVLGGGGQGGGGQGGGGQC |
| | | | | PPCPAPELLGGPSVFLFPPKPKDTLMISRTPEVT |
| | | | | CVVVDVSHEEPEVKFNWYVDGVEVHNAKTKP |
| | | | | CEEQYGSTYRCVSVLTVLHQDWLNGKEYKCK |
| | | | | VSNKALPAPIEKTISKAKGQPREPQVYTLPPSRE |
| | | | | EMTKNQVSLTCLVKGFYPSDIAVEWESNGQPE |
| | | | | NNYKTTPPVLDSDGSFFLYSKLTVDKSRWQQG |
| | | | | NVFSCSVMHEALHNHYTQKSLSLSPGKGGGG |
| | | | | QGGGGQGGGGQGGGGQGGGGQGGGGQCPPC |
| | | | | PAPELLGGPSVFLFPPKPKDTLMISRTPEVTCVV |
| | | | | VDVSHEEPEVKFNWYVDGVEVHNAKTKPCEE |
| | | | | QYGSTYRCVSVLTVLHQDWLNGKEYKCKVSN |
| | | | | KALPAPIEKTISKAKGQPREPQVYTLPPSREEM |
| | | | | TKNQVSLTCLVKGFYPSDIAVEWESNGQPENN |
| | | | | YKTTPPVLDSDGSFFLYSKLTVDKSRWQQGNV |
| | | | | FSCSVMHEALHNHYTQKSLSLSPGKGGGGQG |
| | | | | GGGQGGGGQQVQLVQSGAEVKKPGASVKVSC |
| | | | | KASGYTFTNYWMNWVRQAPGQCLEWMGNIA |
| | | | | YGVKGTNYNQKFQGRVTMTVDTSSSTAYMEL |
| | | | | SRLRSDDTAVYYCATRYFYVMDYWGQGTLVT |
| | | | | VSSGGGGQGGGGQGGGGQDIQMTQSPSSLSAS |
| | | | | VGDRVTITCRASQDISNYLNWYQQKPGKVPKL |
| | | | | LIYYTSRLHSGVPSRFSGSGSGTDFTLTISSLQPE |
| | | | | DVATYYCVQYAQFPLTFGCGTKVEIKSGGGGQ |
| | | | | EVQLVESGGGLVQPGGSLKLSCAASGFTFNKY |
| | | | | AINWVREAPGKGLEWVARIRSKYNNYATYYA |
| | | | | DAVKDRFTISRDDSKNTAYLQMNNLKTEDTA |
| | | | | VYYCVRNANFGTSYISYFAYWGQGTLVTVSSG |
| | | | | GGGQGGGGQGGGGQQTVVTQEPSLTVSPGGT |
| | | | | VTLTCGSSTGAVTSGNYPNWVQKKPGQAPRG |
| | | | | LIGGTKFLAPGTPARFSGSLLGGKAALTLSGVQ |
| | | | | PEDEAEYYCVLWYSNRWVFGSGTKLTVL |
| 283. | MS 15-B12 CC x | artificial | aa | QVQLQESGPGLVKPSETLSLTCTVSGGSISSSSY |
| | I2M2 x G4 x scFc | | | FWGWIRQPPGKCLEWIGNIYYSGSSNYNPSLKS |
| | x G4 x CH3 005- | | | RVTISVDTSKNQFSLKLSSVTAADTAVYYCAR |
| | D5 CC x | | | LPRGDRDAFDIWGQGTMVTVSSGGGGQGGGG |
| | I2M2_GQ - Full | | | QGGGGQDIVMTQSPSSLSASVGDRVTITCRASQ |
| | Sequence | | | GISNYLAWYQQKPGKVPKLLIYAASTLQSGVP |
| | | | | SRFSGSGSGTDFTLTISSLQPEDFATYYCQQSYS |
| | | | | TPFTFGCGTKVEIKSGGGGQEVQLVESGGGLV |
| | | | | QPGGSLKLSCAASGFTFNKYAINWVREAPGKG |
| | | | | LEWVARIRSKYNNYATYYADAVKDRFTISRDD |
| | | | | SKNTAYLQMNNLKTEDTAVYYCVRNANFGTS |
| | | | | YISYFAYWGQGTLVTVSSGGGGQGGGGQGGG |
| | | | | GQQTVVTQEPSLTVSPGGTVTLTCGSSTGAVTS |
| | | | | GNYPNWVQKKPGQAPRGLIGGTKFLAPGTPAR |
| | | | | FSGSLLGGKAALTLSGVQPEDEAEYYCVLWYS |
| | | | | NRWVFGSGTKLTVLGGGGCPPCPAPELLGGPS |
| | | | | VFLFPPKPKDTLMISRTPEVTCVVVDVSHEEPE |
| | | | | VKFNWYVDGVEVHNAKTKPCEEQYGSTYRCV |
| | | | | SVLTVLHQDWLNGKEYKCKVSNKALPAPIEKT |
| | | | | ISKAKGQPREPQVYTLPPSREEMTKNQVSLTCL |
| | | | | VKGFYPSDIAVEWESNGQPENNYKTTPPVLDS |
| | | | | DGSFFLYSKLTVDKSRWQQGNVFSCSVMHEA |
| | | | | LHNHYTQKSLSLSPGKGGGGQGGGGQGGGGQ |
| | | | | GGGGQGGGGQGGGGQCPPCPAPELLGGPSVFL |
| | | | | FPPKPKDTLMISRTPEVTCVVVDVSHEEPEVKF |
| | | | | NWYVDGVEVHNAKTKPCEEQYGSTYRCVSVL |
| | | | | TVLHQDWLNGKEYKCKVSNKALPAPIEKTISK |
| | | | | AKGQPREPQVYTLPPSREEMTKNQVSLTCLVK |
| | | | | GFYPSDIAVEWESNGQPENNYKTTPPVLDSDG |
| | | | | SFFLYSKLTVDKSRWQQGNVFSCSVMHEALH |
| | | | | NHYTQKSLSLSPGKGGGGEVQLLESGGGLVQP |
| | | | | GGSLRLSCAASGFSFSSYPINWVRQAPGKCLE |
| | | | | WVGVIWTGGGTNYASSVKGRFTISRDNSKNTV |
| | | | | YLQMNSLRAEDTAVYYCAKSRGVYDFKGRGA |
| | | | | MDYWGQGTLVTVSSGGGGQGGGGQGGGGQD |
| | | | | IVMTQSPDSLAVSLGERATINCKSSQSLLYSSN |
| | | | | QKNYFAWYQQKPGQPPKLLIYWASTRESGVP |
| | | | | DRFSGSGSGTDFTLTISSLQAEDVAVYYCQQY |
| | | | | YSYPYTFGCGTKLEIKSGGGGQEVQLVESGGG |
| | | | | LVQPGGSLKLSCAASGFTFNKYAINWVREAPG |
| | | | | KGLEWVARIRSKYNNYATYYADAVKDRFTISR |
| | | | | DDSKNTAYLQMNNLKTEDTAVYYCVRNANFG |
| | | | | TSYISYFAYWGQGTLVTVSSGGGGQGGGGQG |
| | | | | GGGQQTVVTQEPSLTVSPGGTVTLTCGSSTGA |
| | | | | VTSGNYPNWVQKKPGQAPRGLIGGTKFLAPGT |
| | | | | PARFSGSLLGGKAALTLSGVQPEDEAEYYCVL |
| | | | | WYSNRWVFGSGTKLTVL |
| 284. | MS 15-B12 CC x | artificial | aa | QVQLQESGPGLVKPSETLSLTCTVSGGSISSSSY |
| | I2M2 x G4 x scFc | | | FWGWIRQPPGKCLEWIGNIYYSGSSNYNPSLKS |
| | x G4 x CH3 22- | | | RVTISVDTSKNQFSLKLSSVTAADTAVYYCAR |
| | A12 CC x | | | LPRGDRDAFDIWGQGTMVTVSSGGGGQGGGG |
| | I2M2_GQ - Full | | | QGGGGQDIVMTQSPSSLSASVGDRVTITCRASQ |
| | Sequence | | | GISNYLAWYQQKPGKVPKLLIYAASTLQSGVP |
| | | | | SRFSGSGSGTDFTLTISSLQPEDFATYYCQQSYS |
| | | | | TPFTFGCGTKVEIKSGGGGQEVQLVESGGGLV |
| | | | | QPGGSLKLSCAASGFTFNKYAINWVREAPGKG |
| | | | | LEWVARIRSKYNNYATYYADAVKDRFTISRDD |
| | | | | SKNTAYLQMNNLKTEDTAVYYCVRNANFGTS |
| | | | | YISYFAYWGQGTLVTVSSGGGGQGGGGQGGG |
| | | | | GQQTVVTQEPSLTVSPGGTVTLTCGSSTGAVTS |
| | | | | GNYPNWVQKKPGQAPRGLIGGTKFLAPGTPAR |
| | | | | FSGSLLGGKAALTLSGVQPEDEAEYYCVLWYS |
| | | | | NRWVFGSGTKLTVLGGGGCPPCPAPELLGGPS |
| | | | | VFLFPPKPKDTLMISRTPEVTCVVVDVSHEEPE |
| | | | | VKFNWYVDGVEVHNAKTKPCEEQYGSTYRCV |
| | | | | SVLTVLHQDWLNGKEYKCKVSNKALPAPIEKT |
| | | | | ISKAKGQPREPQVYTLPPSREEMTKNQVSLTCL |
| | | | | VKGFYPSDIAVEWESNGQPENNYKTTPPVLDS |
| | | | | DGSFFLYSKLTVDKSRWQQGNVFSCSVMHEA |
| | | | | LHNHYTQKSLSLSPGKGGGGQGGGGQGGGGQ |
| | | | | GGGGQGGGGQGGGGQCPPCPAPELLGGPSVFL |
| | | | | FPPKPKDTLMISRTPEVTCVVVDVSHEEPEVKF |
| | | | | NWYVDGVEVHNAKTKPCEEQYGSTYRCVSVL |
| | | | | TVLHQDWLNGKEYKCKVSNKALPAPIEKTISK |
| | | | | AKGQPREPQVYTLPPSREEMTKNQVSLTCLVK |
| | | | | GFYPSDIAVEWESNGQPENNYKTTPPVLDSDG |
| | | | | SFFLYSKLTVDKSRWQQGNVFSCSVMHEALH |
| | | | | NHYTQKSLSLSPGKGGGGQVQLVQSGAEVVK |
| | | | | PGASVKVSCKASGYTFTSSWMNWVRQAPGQC |
| | | | | LEWMGRIYTGTGETKYSGKFQGRVTITRDTSA |
| | | | | STAYMELSSLTSEDTAVYYCARQRDYGALYA |
| | | | | MDYWGQGTLVTVSSGGGGQGGGGQGGGGQD |
| | | | | IQLTQSPSFLSASVGDRVTITCRASDDIYSYLAW |
| | | | | YQQKPGKAPKLLVYNAKTLAEGVPSRFSGSGS |
| | | | | GTEFTLTISSLQPEDFATYYCQNHDRTPFTFGC |
| | | | | GTKVDIKSGGGGQEVQLVESGGGLVQPGGSLK |
| | | | | LSCAASGFTFNKYAINWVREAPGKGLEWVARI |
| | | | | RSKYNNYATYYADAVKDRFTISRDDSKNTAYL |
| | | | | QMNNLKTEDTAVYYCVRNANFGTSYISYFAY |
| | | | | WGQGTLVTVSSGGGGQGGGGQGGGGQQTVV |
| | | | | TQEPSLTVSPGGTVTLTCGSSTGAVTSGNYPN |
| | | | | WVQKKPGQAPRGLIGGTKFLAPGTPARFSGSL |
| | | | | LGGKAALTLSGVQPEDEAEYYCVLWYSNRWV |
| | | | | FGSGTKLTVL |
| 285. | MS 15-B12 CC x | artificial | aa | QVQLQESGPGLVKPSETLSLTCTVSGGSISSSSY |
| | I2M2 x G4 x scFc | | | FWGWIRQPPGKCLEWIGNIYYSGSSNYNPSLKS |
| | x G4 x CH3 26-E5 | | | RVTISVDTSKNQFSLKLSSVTAADTAVYYCAR |
| | CC x I2M2_GQ - | | | LPRGDRDAFDIWGQGTMVTVSSGGGGQGGGG |
| | Full Sequence | | | QGGGGQDIVMTQSPSSLSASVGDRVTITCRASQ |
| | | | | GISNYLAWYQQKPGKVPKLLIYAASTLQSGVP |
| | | | | SRFSGSGSGTDFTLTISSLQPEDFATYYCQQSYS |
| | | | | TPFTFGCGTKVEIKSGGGGQEVQLVESGGGLV |
| | | | | QPGGSLKLSCAASGFTFNKYAINWVREAPGKG |
| | | | | LEWVARIRSKYNNYATYYADAVKDRFTISRDD |
| | | | | SKNTAYLQMNNLKTEDTAVYYCVRNANFGTS |
| | | | | YISYFAYWGQGTLVTVSSGGGGQGGGGQGGG |
| | | | | GQQTVVTQEPSLTVSPGGTVTLTCGSSTGAVTS |
| | | | | GNYPNWVQKKPGQAPRGLIGGTKFLAPGTPAR |
| | | | | FSGSLLGGKAALTLSGVQPEDEAEYYCVLWYS |
| | | | | NRWVFGSGTKLTVLGGGGCPPCPAPELLGGPS |
| | | | | VFLFPPKPKDTLMISRTPEVTCVVVDVSHEEPE |
| | | | | VKFNWYVDGVEVHNAKTKPCEEQYGSTYRCV |
| | | | | SVLTVLHQDWLNGKEYKCKVSNKALPAPIEKT |
| | | | | ISKAKGQPREPQVYTLPPSREEMTKNQVSLTCL |
| | | | | VKGFYPSDIAVEWESNGQPENNYKTTPPVLDS |
| | | | | DGSFFLYSKLTVDKSRWQQGNVFSCSVMHEA |
| | | | | LHNHYTQKSLSLSPGKGGGGQGGGGQGGGGQ |
| | | | | GGGGQGGGGQGGGGQCPPCPAPELLGGPSVFL |
| | | | | FPPKPKDTLMISRTPEVTCVVVDVSHEEPEVKF |
| | | | | NWYVDGVEVHNAKTKPCEEQYGSTYRCVSVL |
| | | | | TVLHQDWLNGKEYKCKVSNKALPAPIEKTISK |
| | | | | AKGQPREPQVYTLPPSREEMTKNQVSLTCLVK |
| | | | | GFYPSDIAVEWESNGQPENNYKTTPPVLDSDG |
| | | | | SFFLYSKLTVDKSRWQQGNVFSCSVMHEALH |
| | | | | NHYTQKSLSLSPGKGGGGQVQLVQSGAEVKK |
| | | | | PGASVKVSCKASGYTFTSYWMHWVRQAPGQC |
| | | | | LEWMGVIRTSTSYTIYNQKFKGRVTMTRDTST |
| | | | | STVYMELSSLRSEDTAVYYCARSGPGYFDVW |
| | | | | GQGTMVTVSSGGGGQGGGGQGGGGQDIQLTQ |
| | | | | SPSFLSASVGDRVTITCRASENIYSYLAWYQQK |
| | | | | PGKAPKLLIYNAKTLAEGVPSRFSGSGSGTEFT |
| | | | | LTISSLQPEDFATYYCQHNYGTPYTFGCGTKLE |
| | | | | IKSGGGGQEVQLVESGGGLVQPGGSLKLSCAA |
| | | | | SGFTFNKYAINWVREAPGKGLEWVARIRSKYN |
| | | | | NYATYYADAVKDRFTISRDDSKNTAYLQMNN |
| | | | | LKTEDTAVYYCVRNANFGTSYISYFAYWGQG |
| | | | | TLVTVSSGGGGQGGGGQGGGGQQTVVTQEPS |
| | | | | LTVSPGGTVTLTCGSSTGAVTSGNYPNWVQKK |
| | | | | PGQAPRGLIGGTKFLAPGTPARFSGSLLGGKAA |
| | | | | LTLSGVQPEDEAEYYCVLWYSNRWVFGSGTK |
| | | | | LTVL |
| 286. | MS 15-B12 CC x | artificial | aa | QVQLQESGPGLVKPSETLSLTCTVSGGSISSSSY |
| | I2M2 x G4S3 x | | | FWGWIRQPPGKCLEWIGNIYYSGSSNYNPSLKS |
| | scFc x G4S3 x | | | RVTISVDTSKNQFSLKLSSVTAADTAVYYCAR |
| | CH3 15-E11 CC x | | | LPRGDRDAFDIWGQGTMVTVSSGGGGSGGGG |
| | I2M2 - Full | | | SGGGGSDIVMTQSPSSLSASVGDRVTITCRASQ |
| | Sequence | | | GISNYLAWYQQKPGKVPKLLIYAASTLQSGVP |
| | | | | SRFSGSGSGTDFTLTISSLQPEDFATYYCQQSYS |
| | | | | TPFTFGCGTKVEIKSGGGGSEVQLVESGGGLV |
| | | | | QPGGSLKLSCAASGFTFNKYAINWVREAPGKG |
| | | | | LEWVARIRSKYNNYATYYADAVKDRFTISRDD |
| | | | | SKNTAYLQMNNLKTEDTAVYYCVRNANFGTS |
| | | | | YISYFAYWGQGTLVTVSSGGGGSGGGGSGGG |
| | | | | GSQTVVTQEPSLTVSPGGTVTLTCGSSTGAVTS |
| | | | | GNYPNWVQKKPGQAPRGLIGGTKFLAPGTPAR |
| | | | | FSGSLLGGKAALTLSGVQPEDEAEYYCVLWYS |
| | | | | NRWVFGSGTKLTVLGGGGSGGGGSGGGGSDK |
| | | | | THTCPPCPAPELLGGPSVFLFPPKPKDTLMISRT |
| | | | | PEVTCVVVDVSHEDPEVKFNWYVDGVEVHNA |
| | | | | KTKPCEEQYGSTYRCVSVLTVLHQDWLNGKE |
| | | | | YKCKVSNKALPAPIEKTISKAKGQPREPQVYTL |
| | | | | PPSREEMTKNQVSLTCLVKGFYPSDIAVEWES |
| | | | | NGQPENNYKTTPPVLDSDGSFFLYSKLTVDKS |
| | | | | RWQQGNVFSCSVMHEALHNHYTQKSLSLSPG |
| | | | | KGGGGSGGGGSGGGGSGGGGSGGGGSGGGGS |
| | | | | DKTHTCPPCPAPELLGGPSVFLFPPKPKDTLMIS |
| | | | | RTPEVTCVVVDVSHEDPEVKFNWYVDGVEVH |
| | | | | NAKTKPCEEQYGSTYRCVSVLTVLHQDWLNG |
| | | | | KEYKCKVSNKALPAPIEKTISKAKGQPREPQVY |
| | | | | TLPPSREEMTKNQVSLTCLVKGFYPSDIAVEWE |
| | | | | SNGQPENNYKTTPPVLDSDGSFFLYSKLTVDKS |
| | | | | RWQQGNVFSCSVMHEALHNHYTQKSLSLSPG |
| | | | | KGGGGSGGGGSGGGGSQVQLVQSGAEVKKPG |
| | | | | ASVKVSCKASGYTFTNYWMNWVRQAPGQCL |
| | | | | EWMGNIAYGVKGTNYNQKFQGRVTMTVDTSS |
| | | | | STAYMELSRLRSDDTAVYYCATRYFYVMDYW |
| | | | | GQGTLVTVSSGGGGSGGGGSGGGGSDIQMTQS |
| | | | | PSSLSASVGDRVTITCRASQDISNYLNWYQQKP |
| | | | | GKVPKLLIYYTSRLHSGVPSRFSGSGSGTDFTL |
| | | | | TISSLQPEDVATYYCVQYAQFPLTFGCGTKVEI |
| | | | | KSGGGGSEVQLVESGGGLVQPGGSLKLSCAAS |
| | | | | GFTFNKYAINWVREAPGKGLEWVARIRSKYN |
| | | | | NYATYYADAVKDRFTISRDDSKNTAYLQMNN |
| | | | | LKTEDTAVYYCVRNANFGTSYISYFAYWGQG |
| | | | | TLVTVSSGGGGSGGGGSGGGGSQTVVTQEPSL |
| | | | | TVSPGGTVTLTCGSSTGAVTSGNYPNWVQKKP |
| | | | | GQAPRGLIGGTKFLAPGTPARFSGSLLGGKAAL |
| | | | | TLSGVQPEDEAEYYCVLWYSNRWVFGSGTKL |
| | | | | TVL |
| 287. | MS 15-B12 CC x | artificial | Aa | QVQLQESGPGLVKPSETLSLTCTVSGGSISSSSY |
| | I2Ccc(44/100)x | | | FWGWIRQPPGKCLEWIGNIYYSGSSNYNPSLKS |
| | (G4S)3x scFcx | | | RVTISVDTSKNQFSLKLSSVTAADTAVYYCAR |
| | (G4S)3x CH3 14- | | | LPRGDRDAFDIWGQGTMVTVSSGGGGSGGGG |
| | D1 CCx | | | SGGGGSDIVMTQSPSSLSASVGDRVTITCRASQ |
| | I2Ccc(44/100) - | | | GISNYLAWYQQKPGKVPKLLIYAASTLQSGVP |
| | Full Sequence | | | SRFSGSGSGTDFTLTISSLQPEDFATYYCQQSYS |
| | | | | TPFTFGCGTKVEIKSGGGGSEVQLVESGGGLV |
| | | | | QPGGSLKLSCAASGFTFNKYAMNWVRQAPGK |
| | | | | CLEWVARIRSKYNNYATYYADSVKDRFTISRD |
| | | | | DSKNTAYLQMNNLKTEDTAVYYCVRHGNFGN |
| | | | | SYISYWAYWGQGTLVTVSSGGGGSGGGGSGG |
| | | | | GGSQTVVTQEPSLTVSPGGTVTLTCGSSTGAVT |
| | | | | SGNYPNWVQQKPGQAPRGLIGGTKFLAPGTPA |
| | | | | RFSGSLLGGKAALTLSGVQPEDEAEYYCVLWY |
| | | | | SNRWVFGCGTKLTVLGGGGSGGGGSGGGGSD |
| | | | | KTHTCPPCPAPELLGGPSVFLFPPKPKDTLMISR |
| | | | | TPEVTCVVVDVSHEDPEVKFNWYVDGVEVHN |
| | | | | AKTKPCEEQYGSTYRCVSVLTVLHQDWLNGK |
| | | | | EYKCKVSNKALPAPIEKTISKAKGQPREPQVYT |
| | | | | LPPSREEMTKNQVSLTCLVKGFYPSDIAVEWES |
| | | | | NGQPENNYKTTPPVLDSDGSFFLYSKLTVDKS |
| | | | | RWQQGNVFSCSVMHEALHNHYTQKSLSLSPG |
| | | | | KGGGGSGGGGSGGGGSGGGGSGGGGSGGGGS |
| | | | | DKTHTCPPCPAPELLGGPSVFLFPPKPKDTLMIS |
| | | | | RTPEVTCVVVDVSHEDPEVKFNWYVDGVEVH |
| | | | | NAKTKPCEEQYGSTYRCVSVLTVLHQDWLNG |
| | | | | KEYKCKVSNKALPAPIEKTISKAKGQPREPQVY |
| | | | | TLPPSREEMTKNQVSLTCLVKGFYPSDIAVEWE |
| | | | | SNGQPENNYKTTPPVLDSDGSFFLYSKLTVDKS |
| | | | | RWQQGNVFSCSVMHEALHNHYTQKSLSLSPG |
| | | | | KGGGGSGGGGSGGGGSQVQLVQSGAEVKKPG |
| | | | | ASVKVSCKASGYTFTSYWMHWVRQAPGQCLE |
| | | | | WMGVIYTSGSYTIYNQKFQGRVTMTRDTSTST |
| | | | | AYMELSSLRSEDTAVYYCARSGPGYFDVWGQ |
| | | | | GTMVTVSSGGGGSGGGGSGGGGSDIQLTQSPS |
| | | | | FLSASVGDRVTITCRASGNIHNYLAWYQQKPG |
| | | | | KAPKLLIYNAKTLAEGVPSRFSGSGSGTEFTLKI |
| | | | | SSLQPEDFATYYCQHFAWTPYTFGCGTKLEIKS |
| | | | | GGGGSEVQLVESGGGLVQPGGSLKLSCAASGF |
| | | | | TFNKYAMNWVRQAPGKCLEWVARIRSKYNN |
| | | | | YATYYADSVKDRFTISRDDSKNTAYLQMNNL |
| | | | | KTEDTAVYYCVRHGNFGNSYISYWAYWGQGT |
| | | | | LVTVSSGGGGSGGGGSGGGGSQTVVTQEPSLT |
| | | | | VSPGGTVTLTCGSSTGAVTSGNYPNWVQQKPG |
| | | | | QAPRGLIGGTKFLAPGTPARFSGSLLGGKAALT |
| | | | | LSGVQPEDEAEYYCVLWYSNRWVFGCGTKLT |
| | | | | VL |
| 288. | MS 15-B12 CC x | artificial | Aa | QVQLQESGPGLVKPSETLSLTCTVSGGSISSSSY |
| | I2L x (G4Q)3 x | | | FWGWIRQPPGKCLEWIGNIYYSGSSNYNPSLKS |
| | scfc x (G4Q)3 x | | | RVTISVDTSKNQFSLKLSSVTAADTAVYYCAR |
| | CH3 005-D5 CC x | | | LPRGDRDAFDIWGQGTMVTVSSGGGGQGGGG |
| | I2L - Full | | | QGGGGQDIVMTQSPSSLSASVGDRVTITCRASQ |
| | Sequence | | | GISNYLAWYQQKPGKVPKLLIYAASTLQSGVP |
| | | | | SRFSGSGSGTDFTLTISSLQPEDFATYYCQQSYS |
| | | | | TPFTFGCGTKVEIKSGGGGQEVQLVESGGGLV |
| | | | | QPGGSLKLSCAASGFTFNKYAMNWVRQAPGK |
| | | | | GMEWVARIRSKYNNYATYYADAVKDRFTISR |
| | | | | DDSKNTLYLQMNNLKTEDTAVYYCVRAGNFG |
| | | | | SSYISYFAYWGQGTLVTVSSGGGGQGGGGQG |
| | | | | GGGQQTVVTQEPSLTVSPGGTVTITCGSSTGAV |
| | | | | TSGNYPNWIQKKPGQAPRGLIGGTKFLAPGTP |
| | | | | ARFSGSLEGGKAALTLSGVQPEDEAEYYCVLY |
| | | | | YSNRWVFGSGTKLTVLGGGGQGGGGQGGGG |
| | | | | QCPPCPAPELLGGPSVFLFPPKPKDTLMISRTPE |
| | | | | VTCVVVDVSHEEPEVKFNWYVDGVEVHNAKT |
| | | | | KPCEEQYGSTYRCVSVLTVLHQDWLNGKEYK |
| | | | | CKVSNKALPAPIEKTISKAKGQPREPQVYTLPP |
| | | | | SREEMTKNQVSLTCLVKGFYPSDIAVEWESNG |
| | | | | QPENNYKTTPPVLDSDGSFFLYSKLTVDKSRW |
| | | | | QQGNVFSCSVMHEALHNHYTQKSLSLSPGKG |
| | | | | GGGQGGGGQGGGGQGGGGQGGGGQGGGGQ |
| | | | | CPPCPAPELLGGPSVFLFPPKPKDTLMISRTPEV |
| | | | | TCVVVDVSHEEPEVKFNWYVDGVEVHNAKTK |
| | | | | PCEEQYGSTYRCVSVLTVLHQDWLNGKEYKC |
| | | | | KVSNKALPAPIEKTISKAKGQPREPQVYTLPPS |
| | | | | REEMTKNQVSLTCLVKGFYPSDIAVEWESNGQ |
| | | | | PENNYKTTPPVLDSDGSFFLYSKLTVDKSRWQ |
| | | | | QGNVFSCSVMHEALHNHYTQKSLSLSPGKGG |
| | | | | GGQGGGGQGGGGQEVQLLESGGGLVQPGGSL |
| | | | | RLSCAASGFSFSSYPINWVRQAPGKCLEWVGVI |
| | | | | WTGGGTNYASSVKGRFTISRDNSKNTVYLQM |
| | | | | NSLRAEDTAVYYCAKSRGVYDFKGRGAMDY |
| | | | | WGQGTLVTVSSGGGGQGGGGQGGGGQDIVM |
| | | | | TQSPDSLAVSLGERATINCKSSQSLLYSSNQKN |
| | | | | YFAWYQQKPGQPPKLLIYWASTRESGVPDRFS |
| | | | | GSGSGTDFTLTISSLQAEDVAVYYCQQYYSYP |
| | | | | YTFGCGTKLEIKSGGGGQEVQLVESGGGLVQP |
| | | | | GGSLKLSCAASGFTFNKYAMNWVRQAPGKG |
| | | | | MEWVARIRSKYNNYATYYADAVKDRFTISRD |
| | | | | DSKNTLYLQMNNLKTEDTAVYYCVRAGNFGS |
| | | | | SYISYFAYWGQGTLVTVSSGGGGQGGGGQGG |
| | | | | GGQQTVVTQEPSLTVSPGGTVTITCGSSTGAVT |
| | | | | SGNYPNWIQKKPGQAPRGLIGGTKFLAPGTPA |
| | | | | RFSGSLEGGKAALTLSGVQPEDEAEYYCVLYY |
| | | | | SNRWVFGSGTKLTVL |
| 289. | MS 15-B12 CC x | artificial | Aa | QVQLQESGPGLVKPSETLSLTCTVSGGSISSSSY |
| | I2L x (G4Q)3 x | | | FWGWIRQPPGKCLEWIGNIYYSGSSNYNPSLKS |
| | scfc x (G4Q)3 x | | | RVTISVDTSKNQFSLKLSSVTAADTAVYYCAR |
| | CH3 26-E5 CC x | | | LPRGDRDAFDIWGQGTMVTVSSGGGGQGGGG |
| | I2L - Full | | | QGGGGQDIVMTQSPSSLSASVGDRVTITCRASQ |
| | Sequence | | | GISNYLAWYQQKPGKVPKLLIYAASTLQSGVP |
| | | | | SRFSGSGSGTDFTLTISSLQPEDFATYYCQQSYS |
| | | | | TPFTFGCGTKVEIKSGGGGQEVQLVESGGGLV |
| | | | | QPGGSLKLSCAASGFTFNKYAMNWVRQAPGK |
| | | | | GMEWVARIRSKYNNYATYYADAVKDRFTISR |
| | | | | DDSKNTLYLQMNNLKTEDTAVYYCVRAGNFG |
| | | | | SSYISYFAYWGQGTLVTVSSGGGGQGGGGQG |
| | | | | GGGQQTVVTQEPSLTVSPGGTVTITCGSSTGAV |
| | | | | TSGNYPNWIQKKPGQAPRGLIGGTKFLAPGTP |
| | | | | ARFSGSLEGGKAALTLSGVQPEDEAEYYCVLY |
| | | | | YSNRWVFGSGTKLTVLGGGGQGGGGQGGGG |
| | | | | QCPPCPAPELLGGPSVFLFPPKPKDTLMISRTPE |
| | | | | VTCVVVDVSHEEPEVKFNWYVDGVEVHNAKT |
| | | | | KPCEEQYGSTYRCVSVLTVLHQDWLNGKEYK |
| | | | | CKVSNKALPAPIEKTISKAKGQPREPQVYTLPP |
| | | | | SREEMTKNQVSLTCLVKGFYPSDIAVEWESNG |
| | | | | QPENNYKTTPPVLDSDGSFFLYSKLTVDKSRW |
| | | | | QQGNVFSCSVMHEALHNHYTQKSLSLSPGKG |
| | | | | GGGQGGGGQGGGGQGGGGQGGGGQGGGGQ |
| | | | | CPPCPAPELLGGPSVFLFPPKPKDTLMISRTPEV |
| | | | | TCVVVDVSHEEPEVKFNWYVDGVEVHNAKTK |
| | | | | PCEEQYGSTYRCVSVLTVLHQDWLNGKEYKC |
| | | | | KVSNKALPAPIEKTISKAKGQPREPQVYTLPPS |
| | | | | REEMTKNQVSLTCLVKGFYPSDIAVEWESNGQ |
| | | | | PENNYKTTPPVLDSDGSFFLYSKLTVDKSRWQ |
| | | | | QGNVFSCSVMHEALHNHYTQKSLSLSPGKGG |
| | | | | GGQGGGGQGGGGQQVQLVQSGAEVKKPGAS |
| | | | | VKVSCKASGYTFTSYWMHWVRQAPGQCLEW |
| | | | | MGVIRTSTSYTIYNQKFKGRVTMTRDTSTSTV |
| | | | | YMELSSLRSEDTAVYYCARSGPGYFDVWGQG |
| | | | | TMVTVSSGGGGQGGGGQGGGGQDIQLTQSPSF |
| | | | | LSASVGDRVTITCRASENIYSYLAWYQQKPGK |
| | | | | APKLLIYNAKTLAEGVPSRFSGSGSGTEFTLTIS |
| | | | | SLQPEDFATYYCQHNYGTPYTFGCGTKLEIKSG |
| | | | | GGGQEVQLVESGGGLVQPGGSLKLSCAASGFT |
| | | | | FNKYAMNWVRQAPGKGMEWVARIRSKYNNY |
| | | | | ATYYADAVKDRFTISRDDSKNTLYLQMNNLK |
| | | | | TEDTAVYYCVRAGNFGSSYISYFAYWGQGTLV |
| | | | | TVSSGGGGQGGGGQGGGGQQTVVTQEPSLTV |
| | | | | SPGGTVTITCGSSTGAVTSGNYPNWIQKKPGQ |
| | | | | APRGLIGGTKFLAPGTPARFSGSLEGGKAALTL |
| | | | | SGVQPEDEAEYYCVLYYSNRWVFGSGTKLTV |
| | | | | L |
| 290. | MS 15-B12 CC x | artificial | Aa | QVQLQESGPGLVKPSETLSLTCTVSGGSISSSSY |
| | I2L x (G4Q)3x | | | FWGWIRQPPGKCLEWIGNIYYSGSSNYNPSLKS |
| | scFcmod x | | | RVTISVDTSKNQFSLKLSSVTAADTAVYYCAR |
| | (G4Q3) x CH3 24- | | | LPRGDRDAFDIWGQGTMVTVSSGGGGQGGGG |
| | D7 CC x I2L - | | | QGGGGQDIVMTQSPSSLSASVGDRVTITCRASQ |
| | Full Sequence | | | GISNYLAWYQQKPGKVPKLLIYAASTLQSGVP |
| | | | | SRFSGSGSGTDFTLTISSLQPEDFATYYCQQSYS |
| | | | | TPFTFGCGTKVEIKSGGGGQEVQLVESGGGLV |
| | | | | QPGGSLKLSCAASGFTFNKYAMNWVRQAPGK |
| | | | | GMEWVARIRSKYNNYATYYADAVKDRFTISR |
| | | | | DDSKNTLYLQMNNLKTEDTAVYYCVRAGNFG |
| | | | | SSYISYFAYWGQGTLVTVSSGGGGQGGGGQG |
| | | | | GGGQQTVVTQEPSLTVSPGGTVTITCGSSTGAV |
| | | | | TSGNYPNWIQKKPGQAPRGLIGGTKFLAPGTP |
| | | | | ARFSGSLEGGKAALTLSGVQPEDEAEYYCVLY |
| | | | | YSNRWVFGSGTKLTVLGGGGQGGGGQGGGG |
| | | | | QCPPCPAPELLGGPSVFLFPPKPKDTLMISRTPE |
| | | | | VTCVVVDVSHEEPEVKFNWYVDGVEVHNAKT |
| | | | | KPCEEQYGSTYRCVSVLTVLHQDWLNGKEYK |
| | | | | CKVSNKALPAPIEKTISKAKGQPREPQVYTLPP |
| | | | | SREEMTKNQVSLTCLVKGFYPSDIAVEWESNG |
| | | | | QPENNYKTTPPVLDSDGSFFLYSKLTVDKSRW |
| | | | | QQGNVFSCSVMHEALHNHYTQKSLSLSPGKG |
| | | | | GGGQGGGGQGGGGQGGGGQGGGGQGGGGQ |
| | | | | CPPCPAPELLGGPSVFLFPPKPKDTLMISRTPEV |
| | | | | TCVVVDVSHEEPEVKFNWYVDGVEVHNAKTK |
| | | | | PCEEQYGSTYRCVSVLTVLHQDWLNGKEYKC |
| | | | | KVSNKALPAPIEKTISKAKGQPREPQVYTLPPS |
| | | | | REEMTKNQVSLTCLVKGFYPSDIAVEWESNGQ |
| | | | | PENNYKTTPPVLDSDGSFFLYSKLTVDKSRWQ |
| | | | | QGNVFSCSVMHEALHNHYTQKSLSLSPGKGG |
| | | | | GGQGGGGQGGGGQQVQLVQSGAEVKKPGAS |
| | | | | VKVSCKASGYTFTNYWMNWVRQAPGQCLEW |
| | | | | MGNIHSKAHGTNYNQKFQGRVTMTVDTSSST |
| | | | | AYMELSRLRSDDTAVYYCATRYFYVMDYWG |
| | | | | QGTLVTVSSGGGGQGGGGQGGGGQDIQMTQS |
| | | | | PSSLSASVGDRVTITCRASQDISNYLNWYQQKP |
| | | | | GKVPKLLIYYTSRLHSGVPSRFSGSGSGTDFTL |
| | | | | TISSLQPEDVATYYCVQYAQFPLTFGCGTKVEI |
| | | | | KSGGGGQEVQLVESGGGLVQPGGSLKLSCAAS |
| | | | | GFTFNKYAMNWVRQAPGKGMEWVARIRSKY |
| | | | | NNYATYYADAVKDRFTISRDDSKNTLYLQMN |
| | | | | NLKTEDTAVYYCVRAGNFGSSYISYFAYWGQ |
| | | | | GTLVTVSSGGGGQGGGGQGGGGQQTVVTQEP |
| | | | | SLTVSPGGTVTITCGSSTGAVTSGNYPNWIQKK |
| | | | | PGQAPRGLIGGTKFLAPGTPARFSGSLEGGKAA |
| | | | | LTLSGVQPEDEAEYYCVLYYSNRWVFGSGTKL |
| | | | | TVL |
| 291. | MS 15-B12 CC x | artificial | Aa | QVQLQESGPGLVKPSETLSLTCTVSGGSISSSSY |
| | I2L x G4 x scFc x | | | FWGWIRQPPGKCLEWIGNIYYSGSSNYNPSLKS |
| | G4 x CH3 15-E11 | | | RVTISVDTSKNQFSLKLSSVTAADTAVYYCAR |
| | CCx I2L - Full | | | LPRGDRDAFDIWGQGTMVTVSSGGGGSGGGG |
| | Sequence | | | SGGGGSDIVMTQSPSSLSASVGDRVTITCRASQ |
| | | | | GISNYLAWYQQKPGKVPKLLIYAASTLQSGVP |
| | | | | SRFSGSGSGTDFTLTISSLQPEDFATYYCQQSYS |
| | | | | TPFTFGCGTKVEIKSGGGGSEVQLVESGGGLV |
| | | | | QPGGSLKLSCAASGFTFNKYAMNWVRQAPGK |
| | | | | GMEWVARIRSKYNNYATYYADAVKDRFTISR |
| | | | | DDSKNTLYLQMNNLKTEDTAVYYCVRAGNFG |
| | | | | SSYISYFAYWGQGTLVTVSSGGGGSGGGGSGG |
| | | | | GGSQTVVTQEPSLTVSPGGTVTITCGSSTGAVT |
| | | | | SGNYPNWIQKKPGQAPRGLIGGTKFLAPGTPA |
| | | | | RFSGSLEGGKAALTLSGVQPEDEAEYYCVLYY |
| | | | | SNRWVFGSGTKLTVLGGGGDKTHTCPPCPAPE |
| | | | | LLGGPSVFLFPPKPKDTLMISRTPEVTCVVVDV |
| | | | | SHEDPEVKFNWYVDGVEVHNAKTKPCEEQYG |
| | | | | STYRCVSVLTVLHQDWLNGKEYKCKVSNKAL |
| | | | | PAPIEKTISKAKGQPREPQVYTLPPSREEMTKN |
| | | | | QVSLTCLVKGFYPSDIAVEWESNGQPENNYKT |
| | | | | TPPVLDSDGSFFLYSKLTVDKSRWQQGNVFSC |
| | | | | SVMHEALHNHYTQKSLSLSPGKGGGGSGGGG |
| | | | | SGGGGSGGGGSGGGGSGGGGSDKTHTCPPCPA |
| | | | | PELLGGPSVFLFPPKPKDTLMISRTPEVTCVVV |
| | | | | DVSHEDPEVKFNWYVDGVEVHNAKTKPCEEQ |
| | | | | YGSTYRCVSVLTVLHQDWLNGKEYKCKVSNK |
| | | | | ALPAPIEKTISKAKGQPREPQVYTLPPSREEMT |
| | | | | KNQVSLTCLVKGFYPSDIAVEWESNGQPENNY |
| | | | | KTTPPVLDSDGSFFLYSKLTVDKSRWQQGNVF |
| | | | | SCSVMHEALHNHYTQKSLSLSPGKGGGGQVQ |
| | | | | LVQSGAEVKKPGASVKVSCKASGYTFTNYWM |
| | | | | NWVRQAPGQCLEWMGNIAYGVKGTNYNQKF |
| | | | | QGRVTMTVDTSSSTAYMELSRLRSDDTAVYY |
| | | | | CATRYFYVMDYWGQGTLVTVSSGGGGSGGG |
| | | | | GSGGGGSDIQMTQSPSSLSASVGDRVTITCRAS |
| | | | | QDISNYLNWYQQKPGKVPKLLIYYTSRLHSGV |
| | | | | PSRFSGSGSGTDFTLTISSLQPEDVATYYCVQY |
| | | | | AQFPLTFGCGTKVEIKSGGGGSEVQLVESGGG |
| | | | | LVQPGGSLKLSCAASGFTFNKYAMNWVRQAP |
| | | | | GKGMEWVARIRSKYNNYATYYADAVKDRFTI |
| | | | | SRDDSKNTLYLQMNNLKTEDTAVYYCVRAGN |
| | | | | FGSSYISYFAYWGQGTLVTVSSGGGGSGGGGS |
| | | | | GGGGSQTVVTQEPSLTVSPGGTVTITCGSSTGA |
| | | | | VTSGNYPNWIQKKPGQAPRGLIGGTKFLAPGT |
| | | | | PARFSGSLEGGKAALTLSGVQPEDEAEYYCVL |
| | | | | YYSNRWVFGSGTKLTVL |
| 292. | MS 15-B12 CC x | artificial | Aa | QVQLQESGPGLVKPSETLSLTCTVSGGSISSSSY |
| | I2L x G4 x scFc x | | | FWGWIRQPPGKCLEWIGNIYYSGSSNYNPSLKS |
| | G4 x CH3 22-A12 | | | RVTISVDTSKNQFSLKLSSVTAADTAVYYCAR |
| | CC x I2L_GQ - | | | LPRGDRDAFDIWGQGTMVTVSSGGGGQGGGG |
| | Full Sequence | | | QGGGGQDIVMTQSPSSLSASVGDRVTITCRASQ |
| | | | | GISNYLAWYQQKPGKVPKLLIYAASTLQSGVP |
| | | | | SRFSGSGSGTDFTLTISSLQPEDFATYYCQQSYS |
| | | | | TPFTFGCGTKVEIKSGGGGQEVQLVESGGGLV |
| | | | | QPGGSLKLSCAASGFTFNKYAMNWVRQAPGK |
| | | | | GMEWVARIRSKYNNYATYYADAVKDRFTISR |
| | | | | DDSKNTLYLQMNNLKTEDTAVYYCVRAGNFG |
| | | | | SSYISYFAYWGQGTLVTVSSGGGGQGGGGQG |
| | | | | GGGQQTVVTQEPSLTVSPGGTVTITCGSSTGAV |
| | | | | TSGNYPNWIQKKPGQAPRGLIGGTKFLAPGTP |
| | | | | ARFSGSLEGGKAALTLSGVQPEDEAEYYCVLY |
| | | | | YSNRWVFGSGTKLTVLGGGGCPPCPAPELLGG |
| | | | | PSVFLFPPKPKDTLMISRTPEVTCVVVDVSHEE |
| | | | | PEVKFNWYVDGVEVHNAKTKPCEEQYGSTYR |
| | | | | CVSVLTVLHQDWLNGKEYKCKVSNKALPAPIE |
| | | | | KTISKAKGQPREPQVYTLPPSREEMTKNQVSLT |
| | | | | CLVKGFYPSDIAVEWESNGQPENNYKTTPPVL |
| | | | | DSDGSFFLYSKLTVDKSRWQQGNVFSCSVMHE |
| | | | | ALHNHYTQKSLSLSPGKGGGGQGGGGQGGGG |
| | | | | QGGGGQGGGGQGGGGQCPPCPAPELLGGPSV |
| | | | | FLFPPKPKDTLMISRTPEVTCVVVDVSHEEPEV |
| | | | | KFNWYVDGVEVHNAKTKPCEEQYGSTYRCVS |
| | | | | VLTVLHQDWLNGKEYKCKVSNKALPAPIEKTI |
| | | | | SKAKGQPREPQVYTLPPSREEMTKNQVSLTCL |
| | | | | VKGFYPSDIAVEWESNGQPENNYKTTPPVLDS |
| | | | | DGSFFLYSKLTVDKSRWQQGNVFSCSVMHEA |
| | | | | LHNHYTQKSLSLSPGKGGGGQVQLVQSGAEV |
| | | | | VKPGASVKVSCKASGYTFTSSWMNWVRQAPG |
| | | | | QCLEWMGRIYTGTGETKYSGKFQGRVTITRDT |
| | | | | SASTAYMELSSLTSEDTAVYYCARQRDYGALY |
| | | | | AMDYWGQGTLVTVSSGGGGQGGGGQGGGGQ |
| | | | | DIQLTQSPSFLSASVGDRVTITCRASDDIYSYLA |
| | | | | WYQQKPGKAPKLLVYNAKTLAEGVPSRFSGS |
| | | | | GSGTEFTLTISSLQPEDFATYYCQNHDRTPFTFG |
| | | | | CGTKVDIKSGGGGQEVQLVESGGGLVQPGGSL |
| | | | | KLSCAASGFTFNKYAMNWVRQAPGKGMEWV |
| | | | | ARIRSKYNNYATYYADAVKDRFTISRDDSKNT |
| | | | | LYLQMNNLKTEDTAVYYCVRAGNFGSSYISYF |
| | | | | AYWGQGTLVTVSSGGGGQGGGGQGGGGQQT |
| | | | | VVTQEPSLTVSPGGTVTITCGSSTGAVTSGNYP |
| | | | | NWIQKKPGQAPRGLIGGTKFLAPGTPARFSGSL |
| | | | | EGGKAALTLSGVQPEDEAEYYCVLYYSNRWV |
| | | | | FGSGTKLTVL |
| 293. | MS 15-B12 CC x | artificial | Aa | QVQLQESGPGLVKPSETLSLTCTVSGGSISSSSY |
| | I2L x G4 x scFc x | | | FWGWIRQPPGKCLEWIGNIYYSGSSNYNPSLKS |
| | G4 x CH3 24-D7 | | | RVTISVDTSKNQFSLKLSSVTAADTAVYYCAR |
| | CC x I2L_GQ - | | | LPRGDRDAFDIWGQGTMVTVSSGGGGQGGGG |
| | Full Sequence | | | QGGGGQDIVMTQSPSSLSASVGDRVTITCRASQ |
| | | | | GISNYLAWYQQKPGKVPKLLIYAASTLQSGVP |
| | | | | SRFSGSGSGTDFTLTISSLQPEDFATYYCQQSYS |
| | | | | TPFTFGCGTKVEIKSGGGGQEVQLVESGGGLV |
| | | | | QPGGSLKLSCAASGFTFNKYAMNWVRQAPGK |
| | | | | GMEWVARIRSKYNNYATYYADAVKDRFTISR |
| | | | | DDSKNTLYLQMNNLKTEDTAVYYCVRAGNFG |
| | | | | SSYISYFAYWGQGTLVTVSSGGGGQGGGGQG |
| | | | | GGGQQTVVTQEPSLTVSPGGTVTITCGSSTGAV |
| | | | | TSGNYPNWIQKKPGQAPRGLIGGTKFLAPGTP |
| | | | | ARFSGSLEGGKAALTLSGVQPEDEAEYYCVLY |
| | | | | YSNRWVFGSGTKLTVLGGGGCPPCPAPELLGG |
| | | | | PSVFLFPPKPKDTLMISRTPEVTCVVVDVSHEE |
| | | | | PEVKFNWYVDGVEVHNAKTKPCEEQYGSTYR |
| | | | | CVSVLTVLHQDWLNGKEYKCKVSNKALPAPIE |
| | | | | KTISKAKGQPREPQVYTLPPSREEMTKNQVSLT |
| | | | | CLVKGFYPSDIAVEWESNGQPENNYKTTPPVL |
| | | | | DSDGSFFLYSKLTVDKSRWQQGNVFSCSVMHE |
| | | | | ALHNHYTQKSLSLSPGKGGGGQGGGGQGGGG |
| | | | | QGGGGQGGGGQGGGGQCPPCPAPELLGGPSV |
| | | | | FLFPPKPKDTLMISRTPEVTCVVVDVSHEEPEV |
| | | | | KFNWYVDGVEVHNAKTKPCEEQYGSTYRCVS |
| | | | | VLTVLHQDWLNGKEYKCKVSNKALPAPIEKTI |
| | | | | SKAKGQPREPQVYTLPPSREEMTKNQVSLTCL |
| | | | | VKGFYPSDIAVEWESNGQPENNYKTTPPVLDS |
| | | | | DGSFFLYSKLTVDKSRWQQGNVFSCSVMHEA |
| | | | | LHNHYTQKSLSLSPGKGGGGQVQLVQSGAEV |
| | | | | KKPGASVKVSCKASGYTFTNYWMNWVRQAP |
| | | | | GQCLEWMGNIHSKAHGTNYNQKFQGRVTMT |
| | | | | VDTSSSTAYMELSRLRSDDTAVYYCATRYFYV |
| | | | | MDYWGQGTLVTVSSGGGGQGGGGQGGGGQD |
| | | | | IQMTQSPSSLSASVGDRVTITCRASQDISNYLN |
| | | | | WYQQKPGKVPKLLIYYTSRLHSGVPSRFSGSGS |
| | | | | GTDFTLTISSLQPEDVATYYCVQYAQFPLTFGC |
| | | | | GTKVEIKSGGGGQEVQLVESGGGLVQPGGSLK |
| | | | | LSCAASGFTFNKYAMNWVRQAPGKGMEWVA |
| | | | | RIRSKYNNYATYYADAVKDRFTISRDDSKNTL |
| | | | | YLQMNNLKTEDTAVYYCVRAGNFGSSYISYFA |
| | | | | YWGQGTLVTVSSGGGGQGGGGQGGGGQQTV |
| | | | | VTQEPSLTVSPGGTVTITCGSSTGAVTSGNYPN |
| | | | | WIQKKPGQAPRGLIGGTKFLAPGTPARFSGSLE |
| | | | | GGKAALTLSGVQPEDEAEYYCVLYYSNRWVF |
| | | | | GSGTKLTVL |
| 294. | MS 15-B12 CC x | artificial | Aa | QVQLQESGPGLVKPSETLSLTCTVSGGSISSSSY |
| | I2L x G4S3 x scFc | | | FWGWIRQPPGKCLEWIGNIYYSGSSNYNPSLKS |
| | x G4S3 x CH3 | | | RVTISVDTSKNQFSLKLSSVTAADTAVYYCAR |
| | 005-D5 CC x I2L - | | | LPRGDRDAFDIWGQGTMVTVSSGGGGSGGGG |
| | Full Sequence | | | SGGGGSDIVMTQSPSSLSASVGDRVTITCRASQ |
| | | | | GISNYLAWYQQKPGKVPKLLIYAASTLQSGVP |
| | | | | SRFSGSGSGTDFTLTISSLQPEDFATYYCQQSYS |
| | | | | TPFTFGCGTKVEIKSGGGGSEVQLVESGGGLV |
| | | | | QPGGSLKLSCAASGFTFNKYAMNWVRQAPGK |
| | | | | GMEWVARIRSKYNNYATYYADAVKDRFTISR |
| | | | | DDSKNTLYLQMNNLKTEDTAVYYCVRAGNFG |
| | | | | SSYISYFAYWGQGTLVTVSSGGGGSGGGGSGG |
| | | | | GGSQTVVTQEPSLTVSPGGTVTITCGSSTGAVT |
| | | | | SGNYPNWIQKKPGQAPRGLIGGTKFLAPGTPA |
| | | | | RFSGSLEGGKAALTLSGVQPEDEAEYYCVLYY |
| | | | | SNRWVFGSGTKLTVLGGGGSGGGGSGGGGSD |
| | | | | KTHTCPPCPAPELLGGPSVFLFPPKPKDTLMISR |
| | | | | TPEVTCVVVDVSHEDPEVKFNWYVDGVEVHN |
| | | | | AKTKPCEEQYGSTYRCVSVLTVLHQDWLNGK |
| | | | | EYKCKVSNKALPAPIEKTISKAKGQPREPQVYT |
| | | | | LPPSREEMTKNQVSLTCLVKGFYPSDIAVEWES |
| | | | | NGQPENNYKTTPPVLDSDGSFFLYSKLTVDKS |
| | | | | RWQQGNVFSCSVMHEALHNHYTQKSLSLSPG |
| | | | | KGGGGSGGGGSGGGGSGGGGSGGGGSGGGGS |
| | | | | DKTHTCPPCPAPELLGGPSVFLFPPKPKDTLMIS |
| | | | | RTPEVTCVVVDVSHEDPEVKFNWYVDGVEVH |
| | | | | NAKTKPCEEQYGSTYRCVSVLTVLHQDWLNG |
| | | | | KEYKCKVSNKALPAPIEKTISKAKGQPREPQVY |
| | | | | TLPPSREEMTKNQVSLTCLVKGFYPSDIAVEWE |
| | | | | SNGQPENNYKTTPPVLDSDGSFFLYSKLTVDKS |
| | | | | RWQQGNVFSCSVMHEALHNHYTQKSLSLSPG |
| | | | | KGGGGSGGGGSGGGGSEVQLLESGGGLVQPG |
| | | | | GSLRLSCAASGFSFSSYPINWVRQAPGKCLEW |
| | | | | VGVIWTGGGTNYASSVKGRFTISRDNSKNTVY |
| | | | | LQMNSLRAEDTAVYYCAKSRGVYDFKGRGA |
| | | | | MDYWGQGTLVTVSSGGGGSGGGGSGGGGSDI |
| | | | | VMTQSPDSLAVSLGERATINCKSSQSLLYSSNQ |
| | | | | KNYFAWYQQKPGQPPKLLIYWASTRESGVPDR |
| | | | | FSGSGSGTDFTLTISSLQAEDVAVYYCQQYYSY |
| | | | | PYTFGCGTKLEIKSGGGGSEVQLVESGGGLVQ |
| | | | | PGGSLKLSCAASGFTFNKYAMNWVRQAPGKG |
| | | | | MEWVARIRSKYNNYATYYADAVKDRFTISRD |
| | | | | DSKNTLYLQMNNLKTEDTAVYYCVRAGNFGS |
| | | | | SYISYFAYWGQGTLVTVSSGGGGSGGGGSGGG |
| | | | | GSQTVVTQEPSLTVSPGGTVTITCGSSTGAVTS |
| | | | | GNYPNWIQKKPGQAPRGLIGGTKFLAPGTPAR |
| | | | | FSGSLEGGKAALTLSGVQPEDEAEYYCVLYYS |
| | | | | NRWVFGSGTKLTVL |
| 295. | MS 15-B12 CC x | artificial | Aa | QVQLQESGPGLVKPSETLSLTCTVSGGSISSSSY |
| | I2M2 x G4 x scFc | | | FWGWIRQPPGKCLEWIGNIYYSGSSNYNPSLKS |
| | xG4 x CH3 24-D7 | | | RVTISVDTSKNQFSLKLSSVTAADTAVYYCAR |
| | CC x I2M2_GQ - | | | LPRGDRDAFDIWGQGTMVTVSSGGGGQGGGG |
| | Full Sequence | | | QGGGGQDIVMTQSPSSLSASVGDRVTITCRASQ |
| | | | | GISNYLAWYQQKPGKVPKLLIYAASTLQSGVP |
| | | | | SRFSGSGSGTDFTLTISSLQPEDFATYYCQQSYS |
| | | | | TPFTFGCGTKVEIKSGGGGQEVQLVESGGGLV |
| | | | | QPGGSLKLSCAASGFTFNKYAINWVREAPGKG |
| | | | | LEWVARIRSKYNNYATYYADAVKDRFTISRDD |
| | | | | SKNTAYLQMNNLKTEDTAVYYCVRNANFGTS |
| | | | | YISYFAYWGQGTLVTVSSGGGGQGGGGQGGG |
| | | | | GQQTVVTQEPSLTVSPGGTVTLTCGSSTGAVTS |
| | | | | GNYPNWVQKKPGQAPRGLIGGTKFLAPGTPAR |
| | | | | FSGSLLGGKAALTLSGVQPEDEAEYYCVLWYS |
| | | | | NRWVFGSGTKLTVLGGGGCPPCPAPELLGGPS |
| | | | | VFLFPPKPKDTLMISRTPEVTCVVVDVSHEEPE |
| | | | | VKFNWYVDGVEVHNAKTKPCEEQYGSTYRCV |
| | | | | SVLTVLHQDWLNGKEYKCKVSNKALPAPIEKT |
| | | | | ISKAKGQPREPQVYTLPPSREEMTKNQVSLTCL |
| | | | | VKGFYPSDIAVEWESNGQPENNYKTTPPVLDS |
| | | | | DGSFFLYSKLTVDKSRWQQGNVFSCSVMHEA |
| | | | | LHNHYTQKSLSLSPGKGGGGQGGGGQGGGGQ |
| | | | | GGGGQGGGGQGGGGQCPPCPAPELLGGPSVFL |
| | | | | FPPKPKDTLMISRTPEVTCVVVDVSHEEPEVKF |
| | | | | NWYVDGVEVHNAKTKPCEEQYGSTYRCVSVL |
| | | | | TVLHQDWLNGKEYKCKVSNKALPAPIEKTISK |
| | | | | AKGQPREPQVYTLPPSREEMTKNQVSLTCLVK |
| | | | | GFYPSDIAVEWESNGQPENNYKTTPPVLDSDG |
| | | | | SFFLYSKLTVDKSRWQQGNVFSCSVMHEALH |
| | | | | NHYTQKSLSLSPGKGGGGQVQLVQSGAEVKK |
| | | | | PGASVKVSCKASGYTFTNYWMNWVRQAPGQ |
| | | | | CLEWMGNIHSKAHGTNYNQKFQGRVTMTVDT |
| | | | | SSSTAYMELSRLRSDDTAVYYCATRYFYVMD |
| | | | | YWGQGTLVTVSSGGGGQGGGGQGGGGQDIQ |
| | | | | MTQSPSSLSASVGDRVTITCRASQDISNYLNWY |
| | | | | QQKPGKVPKLLIYYTSRLHSGVPSRFSGSGSGT |
| | | | | DFTLTISSLQPEDVATYYCVQYAQFPLTFGCGT |
| | | | | KVEIKSGGGGQEVQLVESGGGLVQPGGSLKLS |
| | | | | CAASGFTFNKYAINWVREAPGKGLEWVARIRS |
| | | | | KYNNYATYYADAVKDRFTISRDDSKNTAYLQ |
| | | | | MNNLKTEDTAVYYCVRNANFGTSYISYFAYW |
| | | | | GQGTLVTVSSGGGGQGGGGQGGGGQQTVVT |
| | | | | QEPSLTVSPGGTVTLTCGSSTGAVTSGNYPNW |
| | | | | VQKKPGQAPRGLIGGTKFLAPGTPARFSGSLLG |
| | | | | GKAALTLSGVQPEDEAEYYCVLWYSNRWVFG |
| | | | | SGTKLTVL |
| 296. | MS 15-B12 CC x | artificial | aa | QVQLQESGPGLVKPSETLSLTCTVSGGSISSSSY |
| | I2M2 x (G4Q)3x | | | FWGWIRQPPGKCLEWIGNIYYSGSSNYNPSLKS |
| | scFcmod x | | | RVTISVDTSKNQFSLKLSSVTAADTAVYYCAR |
| | (G4Q)3 x CH3 | | | LPRGDRDAFDIWGQGTMVTVSSGGGGQGGGG |
| | 005-D5 CC x | | | QGGGGQDIVMTQSPSSLSASVGDRVTITCRASQ |
| | I2M2_GQ - Full | | | GISNYLAWYQQKPGKVPKLLIYAASTLQSGVP |
| | Sequence | | | SRFSGSGSGTDFTLTISSLQPEDFATYYCQQSYS |
| | | | | TPFTFGCGTKVEIKSGGGGQEVQLVESGGGLV |
| | | | | QPGGSLKLSCAASGFTFNKYAINWVREAPGKG |
| | | | | LEWVARIRSKYNNYATYYADAVKDRFTISRDD |
| | | | | SKNTAYLQMNNLKTEDTAVYYCVRNANFGTS |
| | | | | YISYFAYWGQGTLVTVSSGGGGQGGGGQGGG |
| | | | | GQQTVVTQEPSLTVSPGGTVTLTCGSSTGAVTS |
| | | | | GNYPNWVQKKPGQAPRGLIGGTKFLAPGTPAR |
| | | | | FSGSLLGGKAALTLSGVQPEDEAEYYCVLWYS |
| | | | | NRWVFGSGTKLTVLGGGGQGGGGQGGGGQC |
| | | | | PPCPAPELLGGPSVFLFPPKPKDTLMISRTPEVT |
| | | | | CVVVDVSHEEPEVKFNWYVDGVEVHNAKTKP |
| | | | | CEEQYGSTYRCVSVLTVLHQDWLNGKEYKCK |
| | | | | VSNKALPAPIEKTISKAKGQPREPQVYTLPPSRE |
| | | | | EMTKNQVSLTCLVKGFYPSDIAVEWESNGQPE |
| | | | | NNYKTTPPVLDSDGSFFLYSKLTVDKSRWQQG |
| | | | | NVFSCSVMHEALHNHYTQKSLSLSPGKGGGG |
| | | | | QGGGGQGGGGQGGGGQGGGGQGGGGQCPPC |
| | | | | PAPELLGGPSVFLFPPKPKDTLMISRTPEVTCVV |
| | | | | VDVSHEEPEVKFNWYVDGVEVHNAKTKPCEE |
| | | | | QYGSTYRCVSVLTVLHQDWLNGKEYKCKVSN |
| | | | | KALPAPIEKTISKAKGQPREPQVYTLPPSREEM |
| | | | | TKNQVSLTCLVKGFYPSDIAVEWESNGQPENN |
| | | | | YKTTPPVLDSDGSFFLYSKLTVDKSRWQQGNV |
| | | | | FSCSVMHEALHNHYTQKSLSLSPGKGGGGQG |
| | | | | GGGQGGGGQEVQLLESGGGLVQPGGSLRLSC |
| | | | | AASGFSFSSYPINWVRQAPGKCLEWVGVIWTG |
| | | | | GGTNYASSVKGRFTISRDNSKNTVYLQMNSLR |
| | | | | AEDTAVYYCAKSRGVYDFKGRGAMDYWGQG |
| | | | | TLVTVSSGGGGQGGGGQGGGGQDIVMTQSPD |
| | | | | SLAVSLGERATINCKSSQSLLYSSNQKNYFAW |
| | | | | YQQKPGQPPKLLIYWASTRESGVPDRFSGSGSG |
| | | | | TDFTLTISSLQAEDVAVYYCQQYYSYPYTFGC |
| | | | | GTKLEIKSGGGGQEVQLVESGGGLVQPGGSLK |
| | | | | LSCAASGFTFNKYAINWVREAPGKGLEWVARI |
| | | | | RSKYNNYATYYADAVKDRFTISRDDSKNTAYL |
| | | | | QMNNLKTEDTAVYYCVRNANFGTSYISYFAY |
| | | | | WGQGTLVTVSSGGGGQGGGGQGGGGQQTVV |
| | | | | TQEPSLTVSPGGTVTLTCGSSTGAVTSGNYPN |
| | | | | WVQKKPGQAPRGLIGGTKFLAPGTPARFSGSL |
| | | | | LGGKAALTLSGVQPEDEAEYYCVLWYSNRWV |
| | | | | FGSGTKLTVL |
| 297. | MS 15-B12 CC x | artificial | aa | QVQLQESGPGLVKPSETLSLTCTVSGGSISSSSY |
| | I2M2 x (G4Q)3x | | | FWGWIRQPPGKCLEWIGNIYYSGSSNYNPSLKS |
| | scFcmod x | | | RVTISVDTSKNQFSLKLSSVTAADTAVYYCAR |
| | (G4Q)3 x CH3 26- | | | LPRGDRDAFDIWGQGTMVTVSSGGGGQGGGG |
| | E5 CCx | | | QGGGGQDIVMTQSPSSLSASVGDRVTITCRASQ |
| | I2M2_GQ - Full | | | GISNYLAWYQQKPGKVPKLLIYAASTLQSGVP |
| | Sequence | | | SRFSGSGSGTDFTLTISSLQPEDFATYYCQQSYS |
| | | | | TPFTFGCGTKVEIKSGGGGQEVQLVESGGGLV |
| | | | | QPGGSLKLSCAASGFTFNKYAINWVREAPGKG |
| | | | | LEWVARIRSKYNNYATYYADAVKDRFTISRDD |
| | | | | SKNTAYLQMNNLKTEDTAVYYCVRNANFGTS |
| | | | | YISYFAYWGQGTLVTVSSGGGGQGGGGQGGG |
| | | | | GQQTVVTQEPSLTVSPGGTVTLTCGSSTGAVTS |
| | | | | GNYPNWVQKKPGQAPRGLIGGTKFLAPGTPAR |
| | | | | FSGSLLGGKAALTLSGVQPEDEAEYYCVLWYS |
| | | | | NRWVFGSGTKLTVLGGGGQGGGGQGGGGQC |
| | | | | PPCPAPELLGGPSVFLFPPKPKDTLMISRTPEVT |
| | | | | CVVVDVSHEEPEVKFNWYVDGVEVHNAKTKP |
| | | | | CEEQYGSTYRCVSVLTVLHQDWLNGKEYKCK |
| | | | | VSNKALPAPIEKTISKAKGQPREPQVYTLPPSRE |
| | | | | EMTKNQVSLTCLVKGFYPSDIAVEWESNGQPE |
| | | | | NNYKTTPPVLDSDGSFFLYSKLTVDKSRWQQG |
| | | | | NVFSCSVMHEALHNHYTQKSLSLSPGKGGGG |
| | | | | QGGGGQGGGGQGGGGQGGGGQGGGGQCPPC |
| | | | | PAPELLGGPSVFLFPPKPKDTLMISRTPEVTCVV |
| | | | | VDVSHEEPEVKFNWYVDGVEVHNAKTKPCEE |
| | | | | QYGSTYRCVSVLTVLHQDWLNGKEYKCKVSN |
| | | | | KALPAPIEKTISKAKGQPREPQVYTLPPSREEM |
| | | | | TKNQVSLTCLVKGFYPSDIAVEWESNGQPENN |
| | | | | YKTTPPVLDSDGSFFLYSKLTVDKSRWQQGNV |
| | | | | FSCSVMHEALHNHYTQKSLSLSPGKGGGGQG |
| | | | | GGGQGGGGQQVQLVQSGAEVKKPGASVKVSC |
| | | | | KASGYTFTSYWMHWVRQAPGQCLEWMGVIR |
| | | | | TSTSYTIYNQKFKGRVTMTRDTSTSTVYMELSS |
| | | | | LRSEDTAVYYCARSGPGYFDVWGQGTMVTVS |
| | | | | SGGGGQGGGGQGGGGQDIQLTQSPSFLSASVG |
| | | | | DRVTITCRASENIYSYLAWYQQKPGKAPKLLIY |
| | | | | NAKTLAEGVPSRFSGSGSGTEFTLTISSLQPEDF |
| | | | | ATYYCQHNYGTPYTFGCGTKLEIKSGGGGQEV |
| | | | | QLVESGGGLVQPGGSLKLSCAASGFTFNKYAI |
| | | | | NWVREAPGKGLEWVARIRSKYNNYATYYAD |
| | | | | AVKDRFTISRDDSKNTAYLQMNNLKTEDTAV |
| | | | | YYCVRNANFGTSYISYFAYWGQGTLVTVSSGG |
| | | | | GGQGGGGQGGGGQQTVVTQEPSLTVSPGGTV |
| | | | | TLTCGSSTGAVTSGNYPNWVQKKPGQAPRGLI |
| | | | | GGTKFLAPGTPARFSGSLLGGKAALTLSGVQPE |
| | | | | DEAEYYCVLWYSNRWVFGSGTKLTVL |
| 298. | MS 15-B12 CCx | artificial | aa | QVQLQESGPGLVKPSETLSLTCTVSGGSISSSSY |
| | 6H10.09x (G4)x | | | FWGWIRQPPGKCLEWIGNIYYSGSSNYNPSLKS |
| | scFcx (G4)x CH3 | | | RVTISVDTSKNQFSLKLSSVTAADTAVYYCAR |
| | 14-D1 CCx | | | LPRGDRDAFDIWGQGTMVTVSSGGGGSGGGG |
| | 6H10.09 - Full | | | SGGGGSDIVMTQSPSSLSASVGDRVTITCRASQ |
| | Sequence | | | GISNYLAWYQQKPGKVPKLLIYAASTLQSGVP |
| | | | | SRFSGSGSGTDFTLTISSLQPEDFATYYCQQSYS |
| | | | | TPFTFGCGTKVEIKSGGGGSEVQLVESGGGLV |
| | | | | QPGGSLKLSCAASGFTFNKYAMNWVRQAPGK |
| | | | | GMEWVARIRSKYNNYATYYADAVKDRFTISR |
| | | | | DDSKNTLYLQMNNLKTEDTAVYYCVRAGNFG |
| | | | | SSYISYFAYWGQGTLVTVSSGGGGSGGGGSGG |
| | | | | GGSQTVVTQEPSLTVSPGGTVTITCGSSTGAVT |
| | | | | SGNYPNWIQKKPGQAPRGLIGGTKFLAPGTPA |
| | | | | RFSGSLEGGKAALTLSGVQPEDEAEYYCVLYY |
| | | | | SNRWVFGSGTKLTVLGGGGDKTHTCPPCPAPE |
| | | | | LLGGPSVFLFPPKPKDTLMISRTPEVTCVVVDV |
| | | | | SHEDPEVKFNWYVDGVEVHNAKTKPCEEQYG |
| | | | | STYRCVSVLTVLHQDWLNGKEYKCKVSNKAL |
| | | | | PAPIEKTISKAKGQPREPQVYTLPPSREEMTKN |
| | | | | QVSLTCLVKGFYPSDIAVEWESNGQPENNYKT |
| | | | | TPPVLDSDGSFFLYSKLTVDKSRWQQGNVFSC |
| | | | | SVMHEALHNHYTQKSLSLSPGKGGGGSGGGG |
| | | | | SGGGGSGGGGSGGGGSGGGGSDKTHTCPPCPA |
| | | | | PELLGGPSVFLFPPKPKDTLMISRTPEVTCVVV |
| | | | | DVSHEDPEVKFNWYVDGVEVHNAKTKPCEEQ |
| | | | | YGSTYRCVSVLTVLHQDWLNGKEYKCKVSNK |
| | | | | ALPAPIEKTISKAKGQPREPQVYTLPPSREEMT |
| | | | | KNQVSLTCLVKGFYPSDIAVEWESNGQPENNY |
| | | | | KTTPPVLDSDGSFFLYSKLTVDKSRWQQGNVF |
| | | | | SCSVMHEALHNHYTQKSLSLSPGKGGGGQVQ |
| | | | | LVQSGAEVKKPGASVKVSCKASGYTFTSYWM |
| | | | | HWVRQAPGQCLEWMGVIYTSGSYTIYNQKFQ |
| | | | | GRVTMTRDTSTSTAYMELSSLRSEDTAVYYCA |
| | | | | RSGPGYFDVWGQGTMVTVSSGGGGSGGGGSG |
| | | | | GGGSDIQLTQSPSFLSASVGDRVTITCRASGNIH |
| | | | | NYLAWYQQKPGKAPKLLIYNAKTLAEGVPSRF |
| | | | | SGSGSGTEFTLKISSLQPEDFATYYCQHFAWTP |
| | | | | YTFGCGTKLEIKSGGGGSEVQLVESGGGLVQP |
| | | | | GGSLKLSCAASGFTFNKYAMNWVRQAPGKG |
| | | | | MEWVARIRSKYNNYATYYADAVKDRFTISRD |
| | | | | DSKNTLYLQMNNLKTEDTAVYYCVRAGNFGS |
| | | | | SYISYFAYWGQGTLVTVSSGGGGSGGGGSGGG |
| | | | | GSQTVVTQEPSLTVSPGGTVTITCGSSTGAVTS |
| | | | | GNYPNWIQKKPGQAPRGLIGGTKFLAPGTPAR |
| | | | | FSGSLEGGKAALTLSGVQPEDEAEYYCVLYYS |
| | | | | NRWVFGSGTKLTVL |
| 299. | MS 15-B12 CCx | artificial | aa | QVQLQESGPGLVKPSETLSLTCTVSGGSISSSSY |
| | 6H10.09x | | | FWGWIRQPPGKCLEWIGNIYYSGSSNYNPSLKS |
| | (G4S)3x scFcx | | | RVTISVDTSKNQFSLKLSSVTAADTAVYYCAR |
| | (G4S)3x CH3 14- | | | LPRGDRDAFDIWGQGTMVTVSSGGGGSGGGG |
| | D1 CCx 6H10.09 - | | | SGGGGSDIVMTQSPSSLSASVGDRVTITCRASQ |
| | Full Sequence | | | GISNYLAWYQQKPGKVPKLLIYAASTLQSGVP |
| | | | | SRFSGSGSGTDFTLTISSLQPEDFATYYCQQSYS |
| | | | | TPFTFGCGTKVEIKSGGGGSEVQLVESGGGLV |
| | | | | QPGGSLKLSCAASGFTFNKYAMNWVRQAPGK |
| | | | | GMEWVARIRSKYNNYATYYADAVKDRFTISR |
| | | | | DDSKNTLYLQMNNLKTEDTAVYYCVRAGNFG |
| | | | | SSYISYFAYWGQGTLVTVSSGGGGSGGGGSGG |
| | | | | GGSQTVVTQEPSLTVSPGGTVTITCGSSTGAVT |
| | | | | SGNYPNWIQKKPGQAPRGLIGGTKFLAPGTPA |
| | | | | RFSGSLEGGKAALTLSGVQPEDEAEYYCVLYY |
| | | | | SNRWVFGSGTKLTVLGGGGSGGGGSGGGGSD |
| | | | | KTHTCPPCPAPELLGGPSVFLFPPKPKDTLMISR |
| | | | | TPEVTCVVVDVSHEDPEVKFNWYVDGVEVHN |
| | | | | AKTKPCEEQYGSTYRCVSVLTVLHQDWLNGK |
| | | | | EYKCKVSNKALPAPIEKTISKAKGQPREPQVYT |
| | | | | LPPSREEMTKNQVSLTCLVKGFYPSDIAVEWES |
| | | | | NGQPENNYKTTPPVLDSDGSFFLYSKLTVDKS |
| | | | | RWQQGNVFSCSVMHEALHNHYTQKSLSLSPG |
| | | | | KGGGGSGGGGSGGGGSGGGGSGGGGSGGGGS |
| | | | | DKTHTCPPCPAPELLGGPSVFLFPPKPKDTLMIS |
| | | | | RTPEVTCVVVDVSHEDPEVKFNWYVDGVEVH |
| | | | | NAKTKPCEEQYGSTYRCVSVLTVLHQDWLNG |
| | | | | KEYKCKVSNKALPAPIEKTISKAKGQPREPQVY |
| | | | | TLPPSREEMTKNQVSLTCLVKGFYPSDIAVEWE |
| | | | | SNGQPENNYKTTPPVLDSDGSFFLYSKLTVDKS |
| | | | | RWQQGNVFSCSVMHEALHNHYTQKSLSLSPG |
| | | | | KGGGGSGGGGSGGGGSQVQLVQSGAEVKKPG |
| | | | | ASVKVSCKASGYTFTSYWMHWVRQAPGQCLE |
| | | | | WMGVIYTSGSYTIYNQKFQGRVTMTRDTSTST |
| | | | | AYMELSSLRSEDTAVYYCARSGPGYFDVWGQ |
| | | | | GTMVTVSSGGGGSGGGGSGGGGSDIQLTQSPS |
| | | | | FLSASVGDRVTITCRASGNIHNYLAWYQQKPG |
| | | | | KAPKLLIYNAKTLAEGVPSRFSGSGSGTEFTLKI |
| | | | | SSLQPEDFATYYCQHFAWTPYTFGCGTKLEIKS |
| | | | | GGGGSEVQLVESGGGLVQPGGSLKLSCAASGF |
| | | | | TFNKYAMNWVRQAPGKGMEWVARIRSKYNN |
| | | | | YATYYADAVKDRFTISRDDSKNTLYLQMNNL |
| | | | | KTEDTAVYYCVRAGNFGSSYISYFAYWGQGTL |
| | | | | VTVSSGGGGSGGGGSGGGGSQTVVTQEPSLTV |
| | | | | SPGGTVTITCGSSTGAVTSGNYPNWIQKKPGQ |
| | | | | APRGLIGGTKFLAPGTPARFSGSLEGGKAALTL |
| | | | | SGVQPEDEAEYYCVLYYSNRWVFGSGTKLTV |
| | | | | L |
| 300. | MS 15-B12 CCx | artificial | aa | QVQLQESGPGLVKPSETLSLTCTVSGGSISSSSY |
| | I2M2 x G4 x scFc | | | FWGWIRQPPGKCLEWIGNIYYSGSSNYNPSLKS |
| | x G4 x CH3 15- | | | RVTISVDTSKNQFSLKLSSVTAADTAVYYCAR |
| | E11 CC x I2M2_ | | | LPRGDRDAFDIWGQGTMVTVSSGGGGQGGGG |
| | GQ - Full | | | QGGGGQDIVMTQSPSSLSASVGDRVTITCRASQ |
| | Sequence | | | GISNYLAWYQQKPGKVPKLLIYAASTLQSGVP |
| | | | | SRFSGSGSGTDFTLTISSLQPEDFATYYCQQSYS |
| | | | | TPFTFGCGTKVEIKSGGGGQEVQLVESGGGLV |
| | | | | QPGGSLKLSCAASGFTFNKYAINWVREAPGKG |
| | | | | LEWVARIRSKYNNYATYYADAVKDRFTISRDD |
| | | | | SKNTAYLQMNNLKTEDTAVYYCVRNANFGTS |
| | | | | YISYFAYWGQGTLVTVSSGGGGQGGGGQGGG |
| | | | | GQQTVVTQEPSLTVSPGGTVTLTCGSSTGAVTS |
| | | | | GNYPNWVQKKPGQAPRGLIGGTKFLAPGTPAR |
| | | | | FSGSLLGGKAALTLSGVQPEDEAEYYCVLWYS |
| | | | | NRWVFGSGTKLTVLGGGGCPPCPAPELLGGPS |
| | | | | VFLFPPKPKDTLMISRTPEVTCVVVDVSHEEPE |
| | | | | VKFNWYVDGVEVHNAKTKPCEEQYGSTYRCV |
| | | | | SVLTVLHQDWLNGKEYKCKVSNKALPAPIEKT |
| | | | | ISKAKGQPREPQVYTLPPSREEMTKNQVSLTCL |
| | | | | VKGFYPSDIAVEWESNGQPENNYKTTPPVLDS |
| | | | | DGSFFLYSKLTVDKSRWQQGNVFSCSVMHEA |
| | | | | LHNHYTQKSLSLSPGKGGGGQGGGGQGGGGQ |
| | | | | GGGGQGGGGQGGGGQCPPCPAPELLGGPSVFL |
| | | | | FPPKPKDTLMISRTPEVTCVVVDVSHEEPEVKF |
| | | | | NWYVDGVEVHNAKTKPCEEQYGSTYRCVSVL |
| | | | | TVLHQDWLNGKEYKCKVSNKALPAPIEKTISK |
| | | | | AKGQPREPQVYTLPPSREEMTKNQVSLTCLVK |
| | | | | GFYPSDIAVEWESNGQPENNYKTTPPVLDSDG |
| | | | | SFFLYSKLTVDKSRWQQGNVFSCSVMHEALH |
| | | | | NHYTQKSLSLSPGKGGGGQVQLVQSGAEVKK |
| | | | | PGASVKVSCKASGYTFTNYWMNWVRQAPGQ |
| | | | | CLEWMGNIAYGVKGTNYNQKFQGRVTMTVD |
| | | | | TSSSTAYMELSRLRSDDTAVYYCATRYFYVMD |
| | | | | YWGQGTLVTVSSGGGGQGGGGQGGGGQDIQ |
| | | | | MTQSPSSLSASVGDRVTITCRASQDISNYLNWY |
| | | | | QQKPGKVPKLLIYYTSRLHSGVPSRFSGSGSGT |
| | | | | DFTLTISSLQPEDVATYYCVQYAQFPLTFGCGT |
| | | | | KVEIKSGGGGQEVQLVESGGGLVQPGGSLKLS |
| | | | | CAASGFTFNKYAINWVREAPGKGLEWVARIRS |
| | | | | KYNNYATYYADAVKDRFTISRDDSKNTAYLQ |
| | | | | MNNLKTEDTAVYYCVRNANFGTSYISYFAYW |
| | | | | GQGTLVTVSSGGGGQGGGGQGGGGQQTVVT |
| | | | | QEPSLTVSPGGTVTLTCGSSTGAVTSGNYPNW |
| | | | | VQKKPGQAPRGLIGGTKFLAPGTPARFSGSLLG |
| | | | | GKAALTLSGVQPEDEAEYYCVLWYSNRWVFG |
| | | | | SGTKLTVL |
| 301. | MS 15-B12 CC x | artificial | aa | QVQLQESGPGLVKPSETLSLTCTVSGGSISSSSY |
| | I2L x G4S3 x scFc | | | FWGWIRQPPGKCLEWIGNIYYSGSSNYNPSLKS |
| | x G4S3 x CH3 | | | RVTISVDTSKNQFSLKLSSVTAADTAVYYCAR |
| | 15-E11 CC x I2 - | | | LPRGDRDAFDIWGQGTMVTVSSGGGGSGGGG |
| | Full Sequence | | | SGGGGSDIVMTQSPSSLSASVGDRVTITCRASQ |
| | | | | GISNYLAWYQQKPGKVPKLLIYAASTLQSGVP |
| | | | | SRFSGSGSGTDFTLTISSLQPEDFATYYCQQSYS |
| | | | | TPFTFGCGTKVEIKSGGGGSEVQLVESGGGLV |
| | | | | QPGGSLKLSCAASGFTFNKYAMNWVRQAPGK |
| | | | | GMEWVARIRSKYNNYATYYADAVKDRFTISR |
| | | | | DDSKNTLYLQMNNLKTEDTAVYYCVRAGNFG |
| | | | | SSYISYFAYWGQGTLVTVSSGGGGSGGGGSGG |
| | | | | GGSQTVVTQEPSLTVSPGGTVTITCGSSTGAVT |
| | | | | SGNYPNWIQKKPGQAPRGLIGGTKFLAPGTPA |
| | | | | RFSGSLEGGKAALTLSGVQPEDEAEYYCVLYY |
| | | | | SNRWVFGSGTKLTVLGGGGSGGGGSGGGGSD |
| | | | | KTHTCPPCPAPELLGGPSVFLFPPKPKDTLMISR |
| | | | | TPEVTCVVVDVSHEDPEVKFNWYVDGVEVHN |
| | | | | AKTKPCEEQYGSTYRCVSVLTVLHQDWLNGK |
| | | | | EYKCKVSNKALPAPIEKTISKAKGQPREPQVYT |
| | | | | LPPSREEMTKNQVSLTCLVKGFYPSDIAVEWES |
| | | | | NGQPENNYKTTPPVLDSDGSFFLYSKLTVDKS |
| | | | | RWQQGNVFSCSVMHEALHNHYTQKSLSLSPG |
| | | | | KGGGGSGGGGSGGGGSGGGGSGGGGSGGGGS |
| | | | | DKTHTCPPCPAPELLGGPSVFLFPPKPKDTLMIS |
| | | | | RTPEVTCVVVDVSHEDPEVKFNWYVDGVEVH |
| | | | | NAKTKPCEEQYGSTYRCVSVLTVLHQDWLNG |
| | | | | KEYKCKVSNKALPAPIEKTISKAKGQPREPQVY |
| | | | | TLPPSREEMTKNQVSLTCLVKGFYPSDIAVEWE |
| | | | | SNGQPENNYKTTPPVLDSDGSFFLYSKLTVDKS |
| | | | | RWQQGNVFSCSVMHEALHNHYTQKSLSLSPG |
| | | | | KGGGGSGGGGSGGGGSQVQLVQSGAEVKKPG |
| | | | | ASVKVSCKASGYTFTNYWMNWVRQAPGQCL |
| | | | | EWMGNIAYGVKGTNYNQKFQGRVTMTVDTSS |
| | | | | STAYMELSRLRSDDTAVYYCATRYFYVMDYW |
| | | | | GQGTLVTVSSGGGGSGGGGSGGGGSDIQMTQS |
| | | | | PSSLSASVGDRVTITCRASQDISNYLNWYQQKP |
| | | | | GKVPKLLIYYTSRLHSGVPSRFSGSGSGTDFTL |
| | | | | TISSLQPEDVATYYCVQYAQFPLTFGCGTKVEI |
| | | | | KSGGGGSEVQLVESGGGLVQPGGSLKLSCAAS |
| | | | | GFTFNKYAMNWVRQAPGKGMEWVARIRSKY |
| | | | | NNYATYYADAVKDRFTISRDDSKNTLYLQMN |
| | | | | NLKTEDTAVYYCVRAGNFGSSYISYFAYWGQ |
| | | | | GTLVTVSSGGGGSGGGGSGGGGSQTVVTQEPS |
| | | | | LTVSPGGTVTITCGSSTGAVTSGNYPNWIQKKP |
| | | | | GQAPRGLIGGTKFLAPGTPARFSGSLEGGKAAL |
| | | | | TLSGVQPEDEAEYYCVLYYSNRWVFGSGTKLT |
| | | | | VL |
| 302. | MS 15-B12 CC x | artificial | aa | QVQLQESGPGLVKPSETLSLTCTVSGGSISSSSY |
| | I2L x G4 x scFc x | | | FWGWIRQPPGKCLEWIGNIYYSGSSNYNPSLKS |
| | G4 x CH3 005-D5 | | | RVTISVDTSKNQFSLKLSSVTAADTAVYYCAR |
| | CC x I2L - Full | | | LPRGDRDAFDIWGQGTMVTVSSGGGGSGGGG |
| | Sequence | | | SGGGGSDIVMTQSPSSLSASVGDRVTITCRASQ |
| | | | | GISNYLAWYQQKPGKVPKLLIYAASTLQSGVP |
| | | | | SRFSGSGSGTDFTLTISSLQPEDFATYYCQQSYS |
| | | | | TPFTFGCGTKVEIKSGGGGSEVQLVESGGGLV |
| | | | | QPGGSLKLSCAASGFTFNKYAMNWVRQAPGK |
| | | | | GMEWVARIRSKYNNYATYYADAVKDRFTISR |
| | | | | DDSKNTLYLQMNNLKTEDTAVYYCVRAGNFG |
| | | | | SSYISYFAYWGQGTLVTVSSGGGGSGGGGSGG |
| | | | | GGSQTVVTQEPSLTVSPGGTVTITCGSSTGAVT |
| | | | | SGNYPNWIQKKPGQAPRGLIGGTKFLAPGTPA |
| | | | | RFSGSLEGGKAALTLSGVQPEDEAEYYCVLYY |
| | | | | SNRWVFGSGTKLTVLGGGGDKTHTCPPCPAPE |
| | | | | LLGGPSVFLFPPKPKDTLMISRTPEVTCVVVDV |
| | | | | SHEDPEVKFNWYVDGVEVHNAKTKPCEEQYG |
| | | | | STYRCVSVLTVLHQDWLNGKEYKCKVSNKAL |
| | | | | PAPIEKTISKAKGQPREPQVYTLPPSREEMTKN |
| | | | | QVSLTCLVKGFYPSDIAVEWESNGQPENNYKT |
| | | | | TPPVLDSDGSFFLYSKLTVDKSRWQQGNVFSC |
| | | | | SVMHEALHNHYTQKSLSLSPGKGGGGSGGGG |
| | | | | SGGGGSGGGGSGGGGSGGGGSDKTHTCPPCPA |
| | | | | PELLGGPSVFLFPPKPKDTLMISRTPEVTCVVV |
| | | | | DVSHEDPEVKFNWYVDGVEVHNAKTKPCEEQ |
| | | | | YGSTYRCVSVLTVLHQDWLNGKEYKCKVSNK |
| | | | | ALPAPIEKTISKAKGQPREPQVYTLPPSREEMT |
| | | | | KNQVSLTCLVKGFYPSDIAVEWESNGQPENNY |
| | | | | KTTPPVLDSDGSFFLYSKLTVDKSRWQQGNVF |
| | | | | SCSVMHEALHNHYTQKSLSLSPGKGGGGEVQL |
| | | | | LESGGGLVQPGGSLRLSCAASGFSFSSYPINWV |
| | | | | RQAPGKCLEWVGVIWTGGGTNYASSVKGRFTI |
| | | | | SRDNSKNTVYLQMNSLRAEDTAVYYCAKSRG |
| | | | | VYDFKGRGAMDYWGQGTLVTVSSGGGGSGG |
| | | | | GGSGGGGSDIVMTQSPDSLAVSLGERATINCKS |
| | | | | SQSLLYSSNQKNYFAWYQQKPGQPPKLLIYWA |
| | | | | STRESGVPDRFSGSGSGTDFTLTISSLQAEDVA |
| | | | | VYYCQQYYSYPYTFGCGTKLEIKSGGGGSEVQ |
| | | | | LVESGGGLVQPGGSLKLSCAASGFTFNKYAMN |
| | | | | WVRQAPGKGMEWVARIRSKYNNYATYYADA |
| | | | | VKDRFTISRDDSKNTLYLQMNNLKTEDTAVYY |
| | | | | CVRAGNFGSSYISYFAYWGQGTLVTVSSGGGG |
| | | | | SGGGGSGGGGSQTVVTQEPSLTVSPGGTVTITC |
| | | | | GSSTGAVTSGNYPNWIQKKPGQAPRGLIGGTK |
| | | | | FLAPGTPARFSGSLEGGKAALTLSGVQPEDEAE |
| | | | | YYCVLYYSNRWVFGSGTKLTVL |
| 303. | MS 15-B12 CC x | artificial | aa | QVQLQESGPGLVKPSETLSLTCTVSGGSISSSSY |
| | I2L x G4 x scFc x | | | FWGWIRQPPGKCLEWIGNIYYSGSSNYNPSLKS |
| | G4 x CH3 22-A12 | | | RVTISVDTSKNQFSLKLSSVTAADTAVYYCAR |
| | CC x I2L - Full | | | LPRGDRDAFDIWGQGTMVTVSSGGGGSGGGG |
| | Sequence | | | SGGGGSDIVMTQSPSSLSASVGDRVTITCRASQ |
| | | | | GISNYLAWYQQKPGKVPKLLIYAASTLQSGVP |
| | | | | SRFSGSGSGTDFTLTISSLQPEDFATYYCQQSYS |
| | | | | TPFTFGCGTKVEIKSGGGGSEVQLVESGGGLV |
| | | | | QPGGSLKLSCAASGFTFNKYAMNWVRQAPGK |
| | | | | GMEWVARIRSKYNNYATYYADAVKDRFTISR |
| | | | | DDSKNTLYLQMNNLKTEDTAVYYCVRAGNFG |
| | | | | SSYISYFAYWGQGTLVTVSSGGGGSGGGGSGG |
| | | | | GGSQTVVTQEPSLTVSPGGTVTITCGSSTGAVT |
| | | | | SGNYPNWIQKKPGQAPRGLIGGTKFLAPGTPA |
| | | | | RFSGSLEGGKAALTLSGVQPEDEAEYYCVLYY |
| | | | | SNRWVFGSGTKLTVLGGGGDKTHTCPPCPAPE |
| | | | | LLGGPSVFLFPPKPKDTLMISRTPEVTCVVVDV |
| | | | | SHEDPEVKFNWYVDGVEVHNAKTKPCEEQYG |
| | | | | STYRCVSVLTVLHQDWLNGKEYKCKVSNKAL |
| | | | | PAPIEKTISKAKGQPREPQVYTLPPSREEMTKN |
| | | | | QVSLTCLVKGFYPSDIAVEWESNGQPENNYKT |
| | | | | TPPVLDSDGSFFLYSKLTVDKSRWQQGNVFSC |
| | | | | SVMHEALHNHYTQKSLSLSPGKGGGGSGGGG |
| | | | | SGGGGSGGGGSGGGGSGGGGSDKTHTCPPCPA |
| | | | | PELLGGPSVFLFPPKPKDTLMISRTPEVTCVVV |
| | | | | DVSHEDPEVKFNWYVDGVEVHNAKTKPCEEQ |
| | | | | YGSTYRCVSVLTVLHQDWLNGKEYKCKVSNK |
| | | | | ALPAPIEKTISKAKGQPREPQVYTLPPSREEMT |
| | | | | KNQVSLTCLVKGFYPSDIAVEWESNGQPENNY |
| | | | | KTTPPVLDSDGSFFLYSKLTVDKSRWQQGNVF |
| | | | | SCSVMHEALHNHYTQKSLSLSPGKGGGGQVQ |
| | | | | LVQSGAEVVKPGASVKVSCKASGYTFTSSWM |
| | | | | NWVRQAPGQCLEWMGRIYTGTGETKYSGKFQ |
| | | | | GRVTITRDTSASTAYMELSSLTSEDTAVYYCAR |
| | | | | QRDYGALYAMDYWGQGTLVTVSSGGGGSGG |
| | | | | GGSGGGGSDIQLTQSPSFLSASVGDRVTITCRA |
| | | | | SDDIYSYLAWYQQKPGKAPKLLVYNAKTLAE |
| | | | | GVPSRFSGSGSGTEFTLTISSLQPEDFATYYCQN |
| | | | | HDRTPFTFGCGTKVDIKSGGGGSEVQLVESGG |
| | | | | GLVQPGGSLKLSCAASGFTFNKYAMNWVRQA |
| | | | | PGKGMEWVARIRSKYNNYATYYADAVKDRFT |
| | | | | ISRDDSKNTLYLQMNNLKTEDTAVYYCVRAG |
| | | | | NFGSSYISYFAYWGQGTLVTVSSGGGGSGGGG |
| | | | | SGGGGSQTVVTQEPSLTVSPGGTVTITCGSSTG |
| | | | | AVTSGNYPNWIQKKPGQAPRGLIGGTKFLAPG |
| | | | | TPARFSGSLEGGKAALTLSGVQPEDEAEYYCV |
| | | | | LYYSNRWVFGSGTKLTVL |
| 304. | MS 15-B12 CC x | artificial | Aa | QVQLQESGPGLVKPSETLSLTCTVSGGSISSSSY |
| | I2L x G4 x scFc x | | | FWGWIRQPPGKCLEWIGNIYYSGSSNYNPSLKS |
| | G4 x CH3 24-D7 | | | RVTISVDTSKNQFSLKLSSVTAADTAVYYCAR |
| | CC x I2L - Full | | | LPRGDRDAFDIWGQGTMVTVSSGGGGSGGGG |
| | Sequence | | | SGGGGSDIVMTQSPSSLSASVGDRVTITCRASQ |
| | | | | GISNYLAWYQQKPGKVPKLLIYAASTLQSGVP |
| | | | | SRFSGSGSGTDFTLTISSLQPEDFATYYCQQSYS |
| | | | | TPFTFGCGTKVEIKSGGGGSEVQLVESGGGLV |
| | | | | QPGGSLKLSCAASGFTFNKYAMNWVRQAPGK |
| | | | | GMEWVARIRSKYNNYATYYADAVKDRFTISR |
| | | | | DDSKNTLYLQMNNLKTEDTAVYYCVRAGNFG |
| | | | | SSYISYFAYWGQGTLVTVSSGGGGSGGGGSGG |
| | | | | GGSQTVVTQEPSLTVSPGGTVTITCGSSTGAVT |
| | | | | SGNYPNWIQKKPGQAPRGLIGGTKFLAPGTPA |
| | | | | RFSGSLEGGKAALTLSGVQPEDEAEYYCVLYY |
| | | | | SNRWVFGSGTKLTVLGGGGDKTHTCPPCPAPE |
| | | | | LLGGPSVFLFPPKPKDTLMISRTPEVTCVVVDV |
| | | | | SHEDPEVKFNWYVDGVEVHNAKTKPCEEQYG |
| | | | | STYRCVSVLTVLHQDWLNGKEYKCKVSNKAL |
| | | | | PAPIEKTISKAKGQPREPQVYTLPPSREEMTKN |
| | | | | QVSLTCLVKGFYPSDIAVEWESNGQPENNYKT |
| | | | | TPPVLDSDGSFFLYSKLTVDKSRWQQGNVFSC |
| | | | | SVMHEALHNHYTQKSLSLSPGKGGGGSGGGG |
| | | | | SGGGGSGGGGSGGGGSGGGGSDKTHTCPPCPA |
| | | | | PELLGGPSVFLFPPKPKDTLMISRTPEVTCVVV |
| | | | | DVSHEDPEVKFNWYVDGVEVHNAKTKPCEEQ |
| | | | | YGSTYRCVSVLTVLHQDWLNGKEYKCKVSNK |
| | | | | ALPAPIEKTISKAKGQPREPQVYTLPPSREEMT |
| | | | | KNQVSLTCLVKGFYPSDIAVEWESNGQPENNY |
| | | | | KTTPPVLDSDGSFFLYSKLTVDKSRWQQGNVF |
| | | | | SCSVMHEALHNHYTQKSLSLSPGKGGGGQVQ |
| | | | | LVQSGAEVKKPGASVKVSCKASGYTFTNYWM |
| | | | | NWVRQAPGQCLEWMGNIHSKAHGTNYNQKF |
| | | | | QGRVTMTVDTSSSTAYMELSRLRSDDTAVYY |
| | | | | CATRYFYVMDYWGQGTLVTVSSGGGGSGGG |
| | | | | GSGGGGSDIQMTQSPSSLSASVGDRVTITCRAS |
| | | | | QDISNYLNWYQQKPGKVPKLLIYYTSRLHSGV |
| | | | | PSRFSGSGSGTDFTLTISSLQPEDVATYYCVQY |
| | | | | AQFPLTFGCGTKVEIKSGGGGSEVQLVESGGG |
| | | | | LVQPGGSLKLSCAASGFTFNKYAMNWVRQAP |
| | | | | GKGMEWVARIRSKYNNYATYYADAVKDRFTI |
| | | | | SRDDSKNTLYLQMNNLKTEDTAVYYCVRAGN |
| | | | | FGSSYISYFAYWGQGTLVTVSSGGGGSGGGGS |
| | | | | GGGGSQTVVTQEPSLTVSPGGTVTITCGSSTGA |
| | | | | VTSGNYPNWIQKKPGQAPRGLIGGTKFLAPGT |
| | | | | PARFSGSLEGGKAALTLSGVQPEDEAEYYCVL |
| | | | | YYSNRWVFGSGTKLTVL |
| 305. | MS 15-B12 CC x | artificial | Aa | QVQLQESGPGLVKPSETLSLTCTVSGGSISSSSY |
| | I2L x G4S3 x scFc | | | FWGWIRQPPGKCLEWIGNIYYSGSSNYNPSLKS |
| | x G4S3 x CH3 22- | | | RVTISVDTSKNQFSLKLSSVTAADTAVYYCAR |
| | A12 CC x I2L - | | | LPRGDRDAFDIWGQGTMVTVSSGGGGSGGGG |
| | Full Sequence | | | SGGGGSDIVMTQSPSSLSASVGDRVTITCRASQ |
| | | | | GISNYLAWYQQKPGKVPKLLIYAASTLQSGVP |
| | | | | SRFSGSGSGTDFTLTISSLQPEDFATYYCQQSYS |
| | | | | TPFTFGCGTKVEIKSGGGGSEVQLVESGGGLV |
| | | | | QPGGSLKLSCAASGFTFNKYAMNWVRQAPGK |
| | | | | GMEWVARIRSKYNNYATYYADAVKDRFTISR |
| | | | | DDSKNTLYLQMNNLKTEDTAVYYCVRAGNFG |
| | | | | SSYISYFAYWGQGTLVTVSSGGGGSGGGGSGG |
| | | | | GGSQTVVTQEPSLTVSPGGTVTITCGSSTGAVT |
| | | | | SGNYPNWIQKKPGQAPRGLIGGTKFLAPGTPA |
| | | | | RFSGSLEGGKAALTLSGVQPEDEAEYYCVLYY |
| | | | | SNRWVFGSGTKLTVLGGGGSGGGGSGGGGSD |
| | | | | KTHTCPPCPAPELLGGPSVFLFPPKPKDTLMISR |
| | | | | TPEVTCVVVDVSHEDPEVKFNWYVDGVEVHN |
| | | | | AKTKPCEEQYGSTYRCVSVLTVLHQDWLNGK |
| | | | | EYKCKVSNKALPAPIEKTISKAKGQPREPQVYT |
| | | | | LPPSREEMTKNQVSLTCLVKGFYPSDIAVEWES |
| | | | | NGQPENNYKTTPPVLDSDGSFFLYSKLTVDKS |
| | | | | RWQQGNVFSCSVMHEALHNHYTQKSLSLSPG |
| | | | | KGGGGSGGGGSGGGGSGGGGSGGGGSGGGGS |
| | | | | DKTHTCPPCPAPELLGGPSVFLFPPKPKDTLMIS |
| | | | | RTPEVTCVVVDVSHEDPEVKFNWYVDGVEVH |
| | | | | NAKTKPCEEQYGSTYRCVSVLTVLHQDWLNG |
| | | | | KEYKCKVSNKALPAPIEKTISKAKGQPREPQVY |
| | | | | TLPPSREEMTKNQVSLTCLVKGFYPSDIAVEWE |
| | | | | SNGQPENNYKTTPPVLDSDGSFFLYSKLTVDKS |
| | | | | RWQQGNVFSCSVMHEALHNHYTQKSLSLSPG |
| | | | | KGGGGSGGGGSGGGGSQVQLVQSGAEVVKPG |
| | | | | ASVKVSCKASGYTFTSSWMNWVRQAPGQCLE |
| | | | | WMGRIYTGTGETKYSGKFQGRVTITRDTSAST |
| | | | | AYMELSSLTSEDTAVYYCARQRDYGALYAMD |
| | | | | YWGQGTLVTVSSGGGGSGGGGSGGGGSDIQL |
| | | | | TQSPSFLSASVGDRVTITCRASDDIYSYLAWYQ |
| | | | | QKPGKAPKLLVYNAKTLAEGVPSRFSGSGSGT |
| | | | | EFTLTISSLQPEDFATYYCQNHDRTPFTFGCGT |
| | | | | KVDIKSGGGGSEVQLVESGGGLVQPGGSLKLS |
| | | | | CAASGFTFNKYAMNWVRQAPGKGMEWVARI |
| | | | | RSKYNNYATYYADAVKDRFTISRDDSKNTLYL |
| | | | | QMNNLKTEDTAVYYCVRAGNFGSSYISYFAY |
| | | | | WGQGTLVTVSSGGGGSGGGGSGGGGSQTVVT |
| | | | | QEPSLTVSPGGTVTITCGSSTGAVTSGNYPNWI |
| | | | | QKKPGQAPRGLIGGTKFLAPGTPARFSGSLEGG |
| | | | | KAALTLSGVQPEDEAEYYCVLYYSNRWVFGS |
| | | | | GTKLTVL |
| 306. | MS 15-B12 CC x | artificial | Aa | QVQLQESGPGLVKPSETLSLTCTVSGGSISSSSY |
| | I2Lx G4 x scFc x | | | FWGWIRQPPGKCLEWIGNIYYSGSSNYNPSLKS |
| | G4 x CH3 26-E5 | | | RVTISVDTSKNQFSLKLSSVTAADTAVYYCAR |
| | CC x I2L - Full | | | LPRGDRDAFDIWGQGTMVTVSSGGGGSGGGG |
| | Sequence | | | SGGGGSDIVMTQSPSSLSASVGDRVTITCRASQ |
| | | | | GISNYLAWYQQKPGKVPKLLIYAASTLQSGVP |
| | | | | SRFSGSGSGTDFTLTISSLQPEDFATYYCQQSYS |
| | | | | TPFTFGCGTKVEIKSGGGGSEVQLVESGGGLV |
| | | | | QPGGSLKLSCAASGFTFNKYAMNWVRQAPGK |
| | | | | GMEWVARIRSKYNNYATYYADAVKDRFTISR |
| | | | | DDSKNTLYLQMNNLKTEDTAVYYCVRAGNFG |
| | | | | SSYISYFAYWGQGTLVTVSSGGGGSGGGGSGG |
| | | | | GGSQTVVTQEPSLTVSPGGTVTITCGSSTGAVT |
| | | | | SGNYPNWIQKKPGQAPRGLIGGTKFLAPGTPA |
| | | | | RFSGSLEGGKAALTLSGVQPEDEAEYYCVLYY |
| | | | | SNRWVFGSGTKLTVLGGGGDKTHTCPPCPAPE |
| | | | | LLGGPSVFLFPPKPKDTLMISRTPEVTCVVVDV |
| | | | | SHEDPEVKFNWYVDGVEVHNAKTKPCEEQYG |
| | | | | STYRCVSVLTVLHQDWLNGKEYKCKVSNKAL |
| | | | | PAPIEKTISKAKGQPREPQVYTLPPSREEMTKN |
| | | | | QVSLTCLVKGFYPSDIAVEWESNGQPENNYKT |
| | | | | TPPVLDSDGSFFLYSKLTVDKSRWQQGNVFSC |
| | | | | SVMHEALHNHYTQKSLSLSPGKGGGGSGGGG |
| | | | | SGGGGSGGGGSGGGGSGGGGSDKTHTCPPCPA |
| | | | | PELLGGPSVFLFPPKPKDTLMISRTPEVTCVVV |
| | | | | DVSHEDPEVKFNWYVDGVEVHNAKTKPCEEQ |
| | | | | YGSTYRCVSVLTVLHQDWLNGKEYKCKVSNK |
| | | | | ALPAPIEKTISKAKGQPREPQVYTLPPSREEMT |
| | | | | KNQVSLTCLVKGFYPSDIAVEWESNGQPENNY |
| | | | | KTTPPVLDSDGSFFLYSKLTVDKSRWQQGNVF |
| | | | | SCSVMHEALHNHYTQKSLSLSPGKGGGGQVQ |
| | | | | LVQSGAEVKKPGASVKVSCKASGYTFTSYWM |
| | | | | HWVRQAPGQCLEWMGVIRTSTSYTIYNQKFK |
| | | | | GRVTMTRDTSTSTVYMELSSLRSEDTAVYYCA |
| | | | | RSGPGYFDVWGQGTMVTVSSGGGGSGGGGSG |
| | | | | GGGSDIQLTQSPSFLSASVGDRVTITCRASENIY |
| | | | | SYLAWYQQKPGKAPKLLIYNAKTLAEGVPSRF |
| | | | | SGSGSGTEFTLTISSLQPEDFATYYCQHNYGTP |
| | | | | YTFGCGTKLEIKSGGGGSEVQLVESGGGLVQP |
| | | | | GGSLKLSCAASGFTFNKYAMNWVRQAPGKG |
| | | | | MEWVARIRSKYNNYATYYADAVKDRFTISRD |
| | | | | DSKNTLYLQMNNLKTEDTAVYYCVRAGNFGS |
| | | | | SYISYFAYWGQGTLVTVSSGGGGSGGGGSGGG |
| | | | | GSQTVVTQEPSLTVSPGGTVTITCGSSTGAVTS |
| | | | | GNYPNWIQKKPGQAPRGLIGGTKFLAPGTPAR |
| | | | | FSGSLEGGKAALTLSGVQPEDEAEYYCVLYYS |
| | | | | NRWVFGSGTKLTVL |
| 307. | MS 15-B12 CC x | artificial | Aa | QVQLQESGPGLVKPSETLSLTCTVSGGSISSSSY |
| | I2M2 x G4 x scfc | | | FWGWIRQPPGKCLEWIGNIYYSGSSNYNPSLKS |
| | x G4 x CH3 22- | | | RVTISVDTSKNQFSLKLSSVTAADTAVYYCAR |
| | A12 CC x I2M2 - | | | LPRGDRDAFDIWGQGTMVTVSSGGGGSGGGG |
| | Full Sequence | | | SGGGGSDIVMTQSPSSLSASVGDRVTITCRASQ |
| | | | | GISNYLAWYQQKPGKVPKLLIYAASTLQSGVP |
| | | | | SRFSGSGSGTDFTLTISSLQPEDFATYYCQQSYS |
| | | | | TPFTFGCGTKVEIKSGGGGSEVQLVESGGGLV |
| | | | | QPGGSLKLSCAASGFTFNKYAINWVREAPGKG |
| | | | | LEWVARIRSKYNNYATYYADAVKDRFTISRDD |
| | | | | SKNTAYLQMNNLKTEDTAVYYCVRNANFGTS |
| | | | | YISYFAYWGQGTLVTVSSGGGGSGGGGSGGG |
| | | | | GSQTVVTQEPSLTVSPGGTVTLTCGSSTGAVTS |
| | | | | GNYPNWVQKKPGQAPRGLIGGTKFLAPGTPAR |
| | | | | FSGSLLGGKAALTLSGVQPEDEAEYYCVLWYS |
| | | | | NRWVFGSGTKLTVLGGGGDKTHTCPPCPAPEL |
| | | | | LGGPSVFLFPPKPKDTLMISRTPEVTCVVVDVS |
| | | | | HEDPEVKFNWYVDGVEVHNAKTKPCEEQYGS |
| | | | | TYRCVSVLTVLHQDWLNGKEYKCKVSNKALP |
| | | | | APIEKTISKAKGQPREPQVYTLPPSREEMTKNQ |
| | | | | VSLTCLVKGFYPSDIAVEWESNGQPENNYKTT |
| | | | | PPVLDSDGSFFLYSKLTVDKSRWQQGNVFSCS |
| | | | | VMHEALHNHYTQKSLSLSPGKGGGGSGGGGS |
| | | | | GGGGSGGGGSGGGGSGGGGSDKTHTCPPCPAP |
| | | | | ELLGGPSVFLFPPKPKDTLMISRTPEVTCVVVD |
| | | | | VSHEDPEVKFNWYVDGVEVHNAKTKPCEEQY |
| | | | | GSTYRCVSVLTVLHQDWLNGKEYKCKVSNKA |
| | | | | LPAPIEKTISKAKGQPREPQVYTLPPSREEMTK |
| | | | | NQVSLTCLVKGFYPSDIAVEWESNGQPENNYK |
| | | | | TTPPVLDSDGSFFLYSKLTVDKSRWQQGNVFS |
| | | | | CSVMHEALHNHYTQKSLSLSPGKGGGGQVQL |
| | | | | VQSGAEVVKPGASVKVSCKASGYTFTSSWMN |
| | | | | WVRQAPGQCLEWMGRIYTGTGETKYSGKFQG |
| | | | | RVTITRDTSASTAYMELSSLTSEDTAVYYCARQ |
| | | | | RDYGALYAMDYWGQGTLVTVSSGGGGSGGG |
| | | | | GSGGGGSDIQLTQSPSFLSASVGDRVTITCRAS |
| | | | | DDIYSYLAWYQQKPGKAPKLLVYNAKTLAEG |
| | | | | VPSRFSGSGSGTEFTLTISSLQPEDFATYYCQNH |
| | | | | DRTPFTFGCGTKVDIKSGGGGSEVQLVESGGG |
| | | | | LVQPGGSLKLSCAASGFTFNKYAINWVREAPG |
| | | | | KGLEWVARIRSKYNNYATYYADAVKDRFTISR |
| | | | | DDSKNTAYLQMNNLKTEDTAVYYCVRNANFG |
| | | | | TSYISYFAYWGQGTLVTVSSGGGGSGGGGSGG |
| | | | | GGSQTVVTQEPSLTVSPGGTVTLTCGSSTGAVT |
| | | | | SGNYPNWVQKKPGQAPRGLIGGTKFLAPGTPA |
| | | | | RFSGSLLGGKAALTLSGVQPEDEAEYYCVLWY |
| | | | | SNRWVFGSGTKLTVL |
| 308. | MS 15-B12 CC x | artificial | Aa | QVQLQESGPGLVKPSETLSLTCTVSGGSISSSSY |
| | I2M2 x G4 x | | | FWGWIRQPPGKCLEWIGNIYYSGSSNYNPSLKS |
| | scfc x G4 x CH3 | | | RVTISVDTSKNQFSLKLSSVTAADTAVYYCAR |
| | 24-D7 CC x I2M2 - | | | LPRGDRDAFDIWGQGTMVTVSSGGGGSGGGG |
| | Full Sequence | | | SGGGGSDIVMTQSPSSLSASVGDRVTITCRASQ |
| | | | | GISNYLAWYQQKPGKVPKLLIYAASTLQSGVP |
| | | | | SRFSGSGSGTDFTLTISSLQPEDFATYYCQQSYS |
| | | | | TPFTFGCGTKVEIKSGGGGSEVQLVESGGGLV |
| | | | | QPGGSLKLSCAASGFTFNKYAINWVREAPGKG |
| | | | | LEWVARIRSKYNNYATYYADAVKDRFTISRDD |
| | | | | SKNTAYLQMNNLKTEDTAVYYCVRNANFGTS |
| | | | | YISYFAYWGQGTLVTVSSGGGGSGGGGSGGG |
| | | | | GSQTVVTQEPSLTVSPGGTVTLTCGSSTGAVTS |
| | | | | GNYPNWVQKKPGQAPRGLIGGTKFLAPGTPAR |
| | | | | FSGSLLGGKAALTLSGVQPEDEAEYYCVLWYS |
| | | | | NRWVFGSGTKLTVLGGGGDKTHTCPPCPAPEL |
| | | | | LGGPSVFLFPPKPKDTLMISRTPEVTCVVVDVS |
| | | | | HEDPEVKFNWYVDGVEVHNAKTKPCEEQYGS |
| | | | | TYRCVSVLTVLHQDWLNGKEYKCKVSNKALP |
| | | | | APIEKTISKAKGQPREPQVYTLPPSREEMTKNQ |
| | | | | VSLTCLVKGFYPSDIAVEWESNGQPENNYKTT |
| | | | | PPVLDSDGSFFLYSKLTVDKSRWQQGNVFSCS |
| | | | | VMHEALHNHYTQKSLSLSPGKGGGGSGGGGS |
| | | | | GGGGSGGGGSGGGGSGGGGSDKTHTCPPCPAP |
| | | | | ELLGGPSVFLFPPKPKDTLMISRTPEVTCVVVD |
| | | | | VSHEDPEVKFNWYVDGVEVHNAKTKPCEEQY |
| | | | | GSTYRCVSVLTVLHQDWLNGKEYKCKVSNKA |
| | | | | LPAPIEKTISKAKGQPREPQVYTLPPSREEMTK |
| | | | | NQVSLTCLVKGFYPSDIAVEWESNGQPENNYK |
| | | | | TTPPVLDSDGSFFLYSKLTVDKSRWQQGNVFS |
| | | | | CSVMHEALHNHYTQKSLSLSPGKGGGGQVQL |
| | | | | VQSGAEVKKPGASVKVSCKASGYTFTNYWMN |
| | | | | WVRQAPGQCLEWMGNIHSKAHGTNYNQKFQ |
| | | | | GRVTMTVDTSSSTAYMELSRLRSDDTAVYYC |
| | | | | ATRYFYVMDYWGQGTLVTVSSGGGGSGGGGS |
| | | | | GGGGSDIQMTQSPSSLSASVGDRVTITCRASQD |
| | | | | ISNYLNWYQQKPGKVPKLLIYYTSRLHSGVPSR |
| | | | | FSGSGSGTDFTLTISSLQPEDVATYYCVQYAQF |
| | | | | PLTFGCGTKVEIKSGGGGSEVQLVESGGGLVQ |
| | | | | PGGSLKLSCAASGFTFNKYAINWVREAPGKGL |
| | | | | EWVARIRSKYNNYATYYADAVKDRFTISRDDS |
| | | | | KNTAYLQMNNLKTEDTAVYYCVRNANFGTSY |
| | | | | ISYFAYWGQGTLVTVSSGGGGSGGGGSGGGGS |
| | | | | QTVVTQEPSLTVSPGGTVTLTCGSSTGAVTSGN |
| | | | | YPNWVQKKPGQAPRGLIGGTKFLAPGTPARFS |
| | | | | GSLLGGKAALTLSGVQPEDEAEYYCVLWYSN |
| | | | | RWVFGSGTKLTVL |
| 309. | MS 15-B12 CC x | artificial | Aa | QVQLQESGPGLVKPSETLSLTCTVSGGSISSSSY |
| | I2M2 x G4 x scfc | | | FWGWIRQPPGKCLEWIGNIYYSGSSNYNPSLKS |
| | x G4 x CH3 005- | | | RVTISVDTSKNQFSLKLSSVTAADTAVYYCAR |
| | D5 CC x I2M2 - | | | LPRGDRDAFDIWGQGTMVTVSSGGGGSGGGG |
| | Full Sequence | | | SGGGGSDIVMTQSPSSLSASVGDRVTITCRASQ |
| | | | | GISNYLAWYQQKPGKVPKLLIYAASTLQSGVP |
| | | | | SRFSGSGSGTDFTLTISSLQPEDFATYYCQQSYS |
| | | | | TPFTFGCGTKVEIKSGGGGSEVQLVESGGGLV |
| | | | | QPGGSLKLSCAASGFTFNKYAINWVREAPGKG |
| | | | | LEWVARIRSKYNNYATYYADAVKDRFTISRDD |
| | | | | SKNTAYLQMNNLKTEDTAVYYCVRNANFGTS |
| | | | | YISYFAYWGQGTLVTVSSGGGGSGGGGSGGG |
| | | | | GSQTVVTQEPSLTVSPGGTVTLTCGSSTGAVTS |
| | | | | GNYPNWVQKKPGQAPRGLIGGTKFLAPGTPAR |
| | | | | FSGSLLGGKAALTLSGVQPEDEAEYYCVLWYS |
| | | | | NRWVFGSGTKLTVLGGGGDKTHTCPPCPAPEL |
| | | | | LGGPSVFLFPPKPKDTLMISRTPEVTCVVVDVS |
| | | | | HEDPEVKFNWYVDGVEVHNAKTKPCEEQYGS |
| | | | | TYRCVSVLTVLHQDWLNGKEYKCKVSNKALP |
| | | | | APIEKTISKAKGQPREPQVYTLPPSREEMTKNQ |
| | | | | VSLTCLVKGFYPSDIAVEWESNGQPENNYKTT |
| | | | | PPVLDSDGSFFLYSKLTVDKSRWQQGNVFSCS |
| | | | | VMHEALHNHYTQKSLSLSPGKGGGGSGGGGS |
| | | | | GGGGSGGGGSGGGGSGGGGSDKTHTCPPCPAP |
| | | | | ELLGGPSVFLFPPKPKDTLMISRTPEVTCVVVD |
| | | | | VSHEDPEVKFNWYVDGVEVHNAKTKPCEEQY |
| | | | | GSTYRCVSVLTVLHQDWLNGKEYKCKVSNKA |
| | | | | LPAPIEKTISKAKGQPREPQVYTLPPSREEMTK |
| | | | | NQVSLTCLVKGFYPSDIAVEWESNGQPENNYK |
| | | | | TTPPVLDSDGSFFLYSKLTVDKSRWQQGNVFS |
| | | | | CSVMHEALHNHYTQKSLSLSPGKGGGGEVQL |
| | | | | LESGGGLVQPGGSLRLSCAASGFSFSSYPINWV |
| | | | | RQAPGKCLEWVGVIWTGGGTNYASSVKGRFTI |
| | | | | SRDNSKNTVYLQMNSLRAEDTAVYYCAKSRG |
| | | | | VYDFKGRGAMDYWGQGTLVTVSSGGGGSGG |
| | | | | GGSGGGGSDIVMTQSPDSLAVSLGERATINCKS |
| | | | | SQSLLYSSNQKNYFAWYQQKPGQPPKLLIYWA |
| | | | | STRESGVPDRFSGSGSGTDFTLTISSLQAEDVA |
| | | | | VYYCQQYYSYPYTFGCGTKLEIKSGGGGSEVQ |
| | | | | LVESGGGLVQPGGSLKLSCAASGFTFNKYAIN |
| | | | | WVREAPGKGLEWVARIRSKYNNYATYYADA |
| | | | | VKDRFTISRDDSKNTAYLQMNNLKTEDTAVY |
| | | | | YCVRNANFGTSYISYFAYWGQGTLVTVSSGGG |
| | | | | GSGGGGSGGGGSQTVVTQEPSLTVSPGGTVTL |
| | | | | TCGSSTGAVTSGNYPNWVQKKPGQAPRGLIGG |
| | | | | TKFLAPGTPARFSGSLLGGKAALTLSGVQPEDE |
| | | | | AEYYCVLWYSNRWVFGSGTKLTVL |
| 310. | MS 15-B12 CC x | artificial | Aa | QVQLQESGPGLVKPSETLSLTCTVSGGSISSSSY |
| | I2M2 x G4 x scfc | | | FWGWIRQPPGKCLEWIGNIYYSGSSNYNPSLKS |
| | x G4 x CH3 26-E5 | | | RVTISVDTSKNQFSLKLSSVTAADTAVYYCAR |
| | CC x I2M2 - Full | | | LPRGDRDAFDIWGQGTMVTVSSGGGGSGGGG |
| | Sequence | | | SGGGGSDIVMTQSPSSLSASVGDRVTITCRASQ |
| | | | | GISNYLAWYQQKPGKVPKLLIYAASTLQSGVP |
| | | | | SRFSGSGSGTDFTLTISSLQPEDFATYYCQQSYS |
| | | | | TPFTFGCGTKVEIKSGGGGSEVQLVESGGGLV |
| | | | | QPGGSLKLSCAASGFTFNKYAINWVREAPGKG |
| | | | | LEWVARIRSKYNNYATYYADAVKDRFTISRDD |
| | | | | SKNTAYLQMNNLKTEDTAVYYCVRNANFGTS |
| | | | | YISYFAYWGQGTLVTVSSGGGGSGGGGSGGG |
| | | | | GSQTVVTQEPSLTVSPGGTVTLTCGSSTGAVTS |
| | | | | GNYPNWVQKKPGQAPRGLIGGTKFLAPGTPAR |
| | | | | FSGSLLGGKAALTLSGVQPEDEAEYYCVLWYS |
| | | | | NRWVFGSGTKLTVLGGGGDKTHTCPPCPAPEL |
| | | | | LGGPSVFLFPPKPKDTLMISRTPEVTCVVVDVS |
| | | | | HEDPEVKFNWYVDGVEVHNAKTKPCEEQYGS |
| | | | | TYRCVSVLTVLHQDWLNGKEYKCKVSNKALP |
| | | | | APIEKTISKAKGQPREPQVYTLPPSREEMTKNQ |
| | | | | VSLTCLVKGFYPSDIAVEWESNGQPENNYKTT |
| | | | | PPVLDSDGSFFLYSKLTVDKSRWQQGNVFSCS |
| | | | | VMHEALHNHYTQKSLSLSPGKGGGGSGGGGS |
| | | | | GGGGSGGGGSGGGGSGGGGSDKTHTCPPCPAP |
| | | | | ELLGGPSVFLFPPKPKDTLMISRTPEVTCVVVD |
| | | | | VSHEDPEVKFNWYVDGVEVHNAKTKPCEEQY |
| | | | | GSTYRCVSVLTVLHQDWLNGKEYKCKVSNKA |
| | | | | LPAPIEKTISKAKGQPREPQVYTLPPSREEMTK |
| | | | | NQVSLTCLVKGFYPSDIAVEWESNGQPENNYK |
| | | | | TTPPVLDSDGSFFLYSKLTVDKSRWQQGNVFS |
| | | | | CSVMHEALHNHYTQKSLSLSPGKGGGGQVQL |
| | | | | VQSGAEVKKPGASVKVSCKASGYTFTSYWMH |
| | | | | WVRQAPGQCLEWMGVIRTSTSYTIYNQKFKG |
| | | | | RVTMTRDTSTSTVYMELSSLRSEDTAVYYCAR |
| | | | | SGPGYFDVWGQGTMVTVSSGGGGSGGGGSGG |
| | | | | GGSDIQLTQSPSFLSASVGDRVTITCRASENIYS |
| | | | | YLAWYQQKPGKAPKLLIYNAKTLAEGVPSRES |
| | | | | GSGSGTEFTLTISSLQPEDFATYYCQHNYGTPY |
| | | | | TFGCGTKLEIKSGGGGSEVQLVESGGGLVQPG |
| | | | | GSLKLSCAASGFTFNKYAINWVREAPGKGLEW |
| | | | | VARIRSKYNNYATYYADAVKDRFTISRDDSKN |
| | | | | TAYLQMNNLKTEDTAVYYCVRNANFGTSYIS |
| | | | | YFAYWGQGTLVTVSSGGGGSGGGGSGGGGSQ |
| | | | | TVVTQEPSLTVSPGGTVTLTCGSSTGAVTSGNY |
| | | | | PNWVQKKPGQAPRGLIGGTKFLAPGTPARFSG |
| | | | | SLLGGKAALTLSGVQPEDEAEYYCVLWYSNR |
| | | | | WVFGSGTKLTVL |
| 311. | MS 15-B12 CC x | artificial | Aa | QVQLQESGPGLVKPSETLSLTCTVSGGSISSSSY |
| | I2M2 x G4S3 x | | | FWGWIRQPPGKCLEWIGNIYYSGSSNYNPSLKS |
| | scFc x G4S3 x | | | RVTISVDTSKNQFSLKLSSVTAADTAVYYCAR |
| | CH3 22-A12 CC x | | | LPRGDRDAFDIWGQGTMVTVSSGGGGSGGGG |
| | I2M2 - Full | | | SGGGGSDIVMTQSPSSLSASVGDRVTITCRASQ |
| | Sequence | | | GISNYLAWYQQKPGKVPKLLIYAASTLQSGVP |
| | | | | SRFSGSGSGTDFTLTISSLQPEDFATYYCQQSYS |
| | | | | TPFTFGCGTKVEIKSGGGGSEVQLVESGGGLV |
| | | | | QPGGSLKLSCAASGFTFNKYAINWVREAPGKG |
| | | | | LEWVARIRSKYNNYATYYADAVKDRFTISRDD |
| | | | | SKNTAYLQMNNLKTEDTAVYYCVRNANFGTS |
| | | | | YISYFAYWGQGTLVTVSSGGGGSGGGGSGGG |
| | | | | GSQTVVTQEPSLTVSPGGTVTLTCGSSTGAVTS |
| | | | | GNYPNWVQKKPGQAPRGLIGGTKFLAPGTPAR |
| | | | | FSGSLLGGKAALTLSGVQPEDEAEYYCVLWYS |
| | | | | NRWVFGSGTKLTVLGGGGSGGGGSGGGGSDK |
| | | | | THTCPPCPAPELLGGPSVFLFPPKPKDTLMISRT |
| | | | | PEVTCVVVDVSHEDPEVKFNWYVDGVEVHNA |
| | | | | KTKPCEEQYGSTYRCVSVLTVLHQDWLNGKE |
| | | | | YKCKVSNKALPAPIEKTISKAKGQPREPQVYTL |
| | | | | PPSREEMTKNQVSLTCLVKGFYPSDIAVEWES |
| | | | | NGQPENNYKTTPPVLDSDGSFFLYSKLTVDKS |
| | | | | RWQQGNVFSCSVMHEALHNHYTQKSLSLSPG |
| | | | | KGGGGSGGGGSGGGGSGGGGSGGGGSGGGGS |
| | | | | DKTHTCPPCPAPELLGGPSVFLFPPKPKDTLMIS |
| | | | | RTPEVTCVVVDVSHEDPEVKFNWYVDGVEVH |
| | | | | NAKTKPCEEQYGSTYRCVSVLTVLHQDWLNG |
| | | | | KEYKCKVSNKALPAPIEKTISKAKGQPREPQVY |
| | | | | TLPPSREEMTKNQVSLTCLVKGFYPSDIAVEWE |
| | | | | SNGQPENNYKTTPPVLDSDGSFFLYSKLTVDKS |
| | | | | RWQQGNVFSCSVMHEALHNHYTQKSLSLSPG |
| | | | | KGGGGSGGGGSGGGGSQVQLVQSGAEVVKPG |
| | | | | ASVKVSCKASGYTFTSSWMNWVRQAPGQCLE |
| | | | | WMGRIYTGTGETKYSGKFQGRVTITRDTSAST |
| | | | | AYMELSSLTSEDTAVYYCARQRDYGALYAMD |
| | | | | YWGQGTLVTVSSGGGGSGGGGSGGGGSDIQL |
| | | | | TQSPSFLSASVGDRVTITCRASDDIYSYLAWYQ |
| | | | | QKPGKAPKLLVYNAKTLAEGVPSRFSGSGSGT |
| | | | | EFTLTISSLQPEDFATYYCQNHDRTPFTFGCGT |
| | | | | KVDIKSGGGGSEVQLVESGGGLVQPGGSLKLS |
| | | | | CAASGFTFNKYAINWVREAPGKGLEWVARIRS |
| | | | | KYNNYATYYADAVKDRFTISRDDSKNTAYLQ |
| | | | | MNNLKTEDTAVYYCVRNANFGTSYISYFAYW |
| | | | | GQGTLVTVSSGGGGSGGGGSGGGGSQTVVTQ |
| | | | | EPSLTVSPGGTVTLTCGSSTGAVTSGNYPNWV |
| | | | | QKKPGQAPRGLIGGTKFLAPGTPARFSGSLLGG |
| | | | | KAALTLSGVQPEDEAEYYCVLWYSNRWVFGS |
| | | | | GTKLTVL |
| 312. | MS 15-B12 CC x | artificial | Aa | QVQLQESGPGLVKPSETLSLTCTVSGGSISSSSY |
| | I2M2 x G4S3 x | | | FWGWIRQPPGKCLEWIGNIYYSGSSNYNPSLKS |
| | scFc x G4S3 x | | | RVTISVDTSKNQFSLKLSSVTAADTAVYYCAR |
| | CH3 005-D5 CC x | | | LPRGDRDAFDIWGQGTMVTVSSGGGGSGGGG |
| | I2M2 - Full | | | SGGGGSDIVMTQSPSSLSASVGDRVTITCRASQ |
| | Sequence | | | GISNYLAWYQQKPGKVPKLLIYAASTLQSGVP |
| | | | | SRFSGSGSGTDFTLTISSLQPEDFATYYCQQSYS |
| | | | | TPFTFGCGTKVEIKSGGGGSEVQLVESGGGLV |
| | | | | QPGGSLKLSCAASGFTFNKYAINWVREAPGKG |
| | | | | LEWVARIRSKYNNYATYYADAVKDRFTISRDD |
| | | | | SKNTAYLQMNNLKTEDTAVYYCVRNANFGTS |
| | | | | YISYFAYWGQGTLVTVSSGGGGSGGGGSGGG |
| | | | | GSQTVVTQEPSLTVSPGGTVTLTCGSSTGAVTS |
| | | | | GNYPNWVQKKPGQAPRGLIGGTKFLAPGTPAR |
| | | | | FSGSLLGGKAALTLSGVQPEDEAEYYCVLWYS |
| | | | | NRWVFGSGTKLTVLGGGGSGGGGSGGGGSDK |
| | | | | THTCPPCPAPELLGGPSVFLFPPKPKDTLMISRT |
| | | | | PEVTCVVVDVSHEDPEVKFNWYVDGVEVHNA |
| | | | | KTKPCEEQYGSTYRCVSVLTVLHQDWLNGKE |
| | | | | YKCKVSNKALPAPIEKTISKAKGQPREPQVYTL |
| | | | | PPSREEMTKNQVSLTCLVKGFYPSDIAVEWES |
| | | | | NGQPENNYKTTPPVLDSDGSFFLYSKLTVDKS |
| | | | | RWQQGNVFSCSVMHEALHNHYTQKSLSLSPG |
| | | | | KGGGGSGGGGSGGGGSGGGGSGGGGSGGGGS |
| | | | | DKTHTCPPCPAPELLGGPSVFLFPPKPKDTLMIS |
| | | | | RTPEVTCVVVDVSHEDPEVKFNWYVDGVEVH |
| | | | | NAKTKPCEEQYGSTYRCVSVLTVLHQDWLNG |
| | | | | KEYKCKVSNKALPAPIEKTISKAKGQPREPQVY |
| | | | | TLPPSREEMTKNQVSLTCLVKGFYPSDIAVEWE |
| | | | | SNGQPENNYKTTPPVLDSDGSFFLYSKLTVDKS |
| | | | | RWQQGNVFSCSVMHEALHNHYTQKSLSLSPG |
| | | | | KGGGGSGGGGSGGGGSEVQLLESGGGLVQPG |
| | | | | GSLRLSCAASGFSFSSYPINWVRQAPGKCLEW |
| | | | | VGVIWTGGGTNYASSVKGRFTISRDNSKNTVY |
| | | | | LQMNSLRAEDTAVYYCAKSRGVYDFKGRGA |
| | | | | MDYWGQGTLVTVSSGGGGSGGGGSGGGGSDI |
| | | | | VMTQSPDSLAVSLGERATINCKSSQSLLYSSNQ |
| | | | | KNYFAWYQQKPGQPPKLLIYWASTRESGVPDR |
| | | | | FSGSGSGTDFTLTISSLQAEDVAVYYCQQYYSY |
| | | | | PYTFGCGTKLEIKSGGGGSEVQLVESGGGLVQ |
| | | | | PGGSLKLSCAASGFTFNKYAINWVREAPGKGL |
| | | | | EWVARIRSKYNNYATYYADAVKDRFTISRDDS |
| | | | | KNTAYLQMNNLKTEDTAVYYCVRNANFGTSY |
| | | | | ISYFAYWGQGTLVTVSSGGGGSGGGGSGGGGS |
| | | | | QTVVTQEPSLTVSPGGTVTLTCGSSTGAVTSGN |
| | | | | YPNWVQKKPGQAPRGLIGGTKFLAPGTPARFS |
| | | | | GSLLGGKAALTLSGVQPEDEAEYYCVLWYSN |
| | | | | RWVFGSGTKLTVL |
| 313. | MS 15-B12 CC x | artificial | aa | QVQLQESGPGLVKPSETLSLTCTVSGGSISSSSY |
| | I2M2 x G4S3 x | | | FWGWIRQPPGKCLEWIGNIYYSGSSNYNPSLKS |
| | scFc x G4S3 x | | | RVTISVDTSKNQFSLKLSSVTAADTAVYYCAR |
| | CH3 26-E5 CC x | | | LPRGDRDAFDIWGQGTMVTVSSGGGGSGGGG |
| | I2M2 - Full | | | SGGGGSDIVMTQSPSSLSASVGDRVTITCRASQ |
| | Sequence | | | GISNYLAWYQQKPGKVPKLLIYAASTLQSGVP |
| | | | | SRFSGSGSGTDFTLTISSLQPEDFATYYCQQSYS |
| | | | | TPFTFGCGTKVEIKSGGGGSEVQLVESGGGLV |
| | | | | QPGGSLKLSCAASGFTFNKYAINWVREAPGKG |
| | | | | LEWVARIRSKYNNYATYYADAVKDRFTISRDD |
| | | | | SKNTAYLQMNNLKTEDTAVYYCVRNANFGTS |
| | | | | YISYFAYWGQGTLVTVSSGGGGSGGGGSGGG |
| | | | | GSQTVVTQEPSLTVSPGGTVTLTCGSSTGAVTS |
| | | | | GNYPNWVQKKPGQAPRGLIGGTKFLAPGTPAR |
| | | | | FSGSLLGGKAALTLSGVQPEDEAEYYCVLWYS |
| | | | | NRWVFGSGTKLTVLGGGGSGGGGSGGGGSDK |
| | | | | THTCPPCPAPELLGGPSVFLFPPKPKDTLMISRT |
| | | | | PEVTCVVVDVSHEDPEVKFNWYVDGVEVHNA |
| | | | | KTKPCEEQYGSTYRCVSVLTVLHQDWLNGKE |
| | | | | YKCKVSNKALPAPIEKTISKAKGQPREPQVYTL |
| | | | | PPSREEMTKNQVSLTCLVKGFYPSDIAVEWES |
| | | | | NGQPENNYKTTPPVLDSDGSFFLYSKLTVDKS |
| | | | | RWQQGNVFSCSVMHEALHNHYTQKSLSLSPG |
| | | | | KGGGGSGGGGSGGGGSGGGGSGGGGSGGGGS |
| | | | | DKTHTCPPCPAPELLGGPSVFLFPPKPKDTLMIS |
| | | | | RTPEVTCVVVDVSHEDPEVKFNWYVDGVEVH |
| | | | | NAKTKPCEEQYGSTYRCVSVLTVLHQDWLNG |
| | | | | KEYKCKVSNKALPAPIEKTISKAKGQPREPQVY |
| | | | | TLPPSREEMTKNQVSLTCLVKGFYPSDIAVEWE |
| | | | | SNGQPENNYKTTPPVLDSDGSFFLYSKLTVDKS |
| | | | | RWQQGNVFSCSVMHEALHNHYTQKSLSLSPG |
| | | | | KGGGGSGGGGSGGGGSQVQLVQSGAEVKKPG |
| | | | | ASVKVSCKASGYTFTSYWMHWVRQAPGQCLE |
| | | | | WMGVIRTSTSYTIYNQKFKGRVTMTRDTSTST |
| | | | | VYMELSSLRSEDTAVYYCARSGPGYFDVWGQ |
| | | | | GTMVTVSSGGGGSGGGGSGGGGSDIQLTQSPS |
| | | | | FLSASVGDRVTITCRASENIYSYLAWYQQKPG |
| | | | | KAPKLLIYNAKTLAEGVPSRFSGSGSGTEFTLTI |
| | | | | SSLQPEDFATYYCQHNYGTPYTFGCGTKLEIKS |
| | | | | GGGGSEVQLVESGGGLVQPGGSLKLSCAASGF |
| | | | | TFNKYAINWVREAPGKGLEWVARIRSKYNNY |
| | | | | ATYYADAVKDRFTISRDDSKNTAYLQMNNLK |
| | | | | TEDTAVYYCVRNANFGTSYISYFAYWGQGTL |
| | | | | VTVSSGGGGSGGGGSGGGGSQTVVTQEPSLTV |
| | | | | SPGGTVTLTCGSSTGAVTSGNYPNWVQKKPGQ |
| | | | | APRGLIGGTKFLAPGTPARFSGSLLGGKAALTL |
| | | | | SGVQPEDEAEYYCVLWYSNRWVFGSGTKLTV |
| | | | | L |
| 314. | MS 15-B12 CC x | artificial | aa | QVQLQESGPGLVKPSETLSLTCTVSGGSISSSSY |
| | I2M2 xG4S3 x | | | FWGWIRQPPGKCLEWIGNIYYSGSSNYNPSLKS |
| | scFc x G4S3 - | | | RVTISVDTSKNQFSLKLSSVTAADTAVYYCAR |
| | CH3 24-D7CC x | | | LPRGDRDAFDIWGQGTMVTVSSGGGGSGGGG |
| | I2M2 - Full | | | SGGGGSDIVMTQSPSSLSASVGDRVTITCRASQ |
| | Sequence | | | GISNYLAWYQQKPGKVPKLLIYAASTLQSGVP |
| | | | | SRFSGSGSGTDFTLTISSLQPEDFATYYCQQSYS |
| | | | | TPFTFGCGTKVEIKSGGGGSEVQLVESGGGLV |
| | | | | QPGGSLKLSCAASGFTFNKYAINWVREAPGKG |
| | | | | LEWVARIRSKYNNYATYYADAVKDRFTISRDD |
| | | | | SKNTAYLQMNNLKTEDTAVYYCVRNANFGTS |
| | | | | YISYFAYWGQGTLVTVSSGGGGSGGGGSGGG |
| | | | | GSQTVVTQEPSLTVSPGGTVTLTCGSSTGAVTS |
| | | | | GNYPNWVQKKPGQAPRGLIGGTKFLAPGTPAR |
| | | | | FSGSLLGGKAALTLSGVQPEDEAEYYCVLWYS |
| | | | | NRWVFGSGTKLTVLGGGGSGGGGSGGGGSDK |
| | | | | THTCPPCPAPELLGGPSVFLFPPKPKDTLMISRT |
| | | | | PEVTCVVVDVSHEDPEVKFNWYVDGVEVHNA |
| | | | | KTKPCEEQYGSTYRCVSVLTVLHQDWLNGKE |
| | | | | YKCKVSNKALPAPIEKTISKAKGQPREPQVYTL |
| | | | | PPSREEMTKNQVSLTCLVKGFYPSDIAVEWES |
| | | | | NGQPENNYKTTPPVLDSDGSFFLYSKLTVDKS |
| | | | | RWQQGNVFSCSVMHEALHNHYTQKSLSLSPG |
| | | | | KGGGGSGGGGSGGGGSGGGGSGGGGSGGGGS |
| | | | | DKTHTCPPCPAPELLGGPSVFLFPPKPKDTLMIS |
| | | | | RTPEVTCVVVDVSHEDPEVKFNWYVDGVEVH |
| | | | | NAKTKPCEEQYGSTYRCVSVLTVLHQDWLNG |
| | | | | KEYKCKVSNKALPAPIEKTISKAKGQPREPQVY |
| | | | | TLPPSREEMTKNQVSLTCLVKGFYPSDIAVEWE |
| | | | | SNGQPENNYKTTPPVLDSDGSFFLYSKLTVDKS |
| | | | | RWQQGNVFSCSVMHEALHNHYTQKSLSLSPG |
| | | | | KGGGGSGGGGSGGGGSQVQLVQSGAEVKKPG |
| | | | | ASVKVSCKASGYTFTNYWMNWVRQAPGQCL |
| | | | | EWMGNIHSKAHGTNYNQKFQGRVTMTVDTSS |
| | | | | STAYMELSRLRSDDTAVYYCATRYFYVMDYW |
| | | | | GQGTLVTVSSGGGGSGGGGSGGGGSDIQMTQS |
| | | | | PSSLSASVGDRVTITCRASQDISNYLNWYQQKP |
| | | | | GKVPKLLIYYTSRLHSGVPSRFSGSGSGTDFTL |
| | | | | TISSLQPEDVATYYCVQYAQFPLTFGCGTKVEI |
| | | | | KSGGGGSEVQLVESGGGLVQPGGSLKLSCAAS |
| | | | | GFTFNKYAINWVREAPGKGLEWVARIRSKYN |
| | | | | NYATYYADAVKDRFTISRDDSKNTAYLQMNN |
| | | | | LKTEDTAVYYCVRNANFGTSYISYFAYWGQG |
| | | | | TLVTVSSGGGGSGGGGSGGGGSQTVVTQEPSL |
| | | | | TVSPGGTVTLTCGSSTGAVTSGNYPNWVQKKP |
| | | | | GQAPRGLIGGTKFLAPGTPARFSGSLLGGKAAL |
| | | | | TLSGVQPEDEAEYYCVLWYSNRWVFGSGTKL |
| | | | | TVL |
| 315. | MS 15-B12 CCx | artificial | aa | QVQLQESGPGLVKPSETLSLTCTVSGGSISSSSY |
| | I2C 44/100cc x | | | FWGWIRQPPGKCLEWIGNIYYSGSSNYNPSLKS |
| | scFc x CH3 15- | | | RVTISVDTSKNQFSLKLSSVTAADTAVYYCAR |
| | E11 CC x | | | LPRGDRDAFDIWGQGTMVTVSSGGGGSGGGG |
| | I2C4/100cc0 - | | | SGGGGSDIVMTQSPSSLSASVGDRVTITCRASQ |
| | Full Sequence | | | GISNYLAWYQQKPGKVPKLLIYAASTLQSGVP |
| | | | | SRFSGSGSGTDFTLTISSLQPEDFATYYCQQSYS |
| | | | | TPFTFGCGTKVEIKSGGGGSEVQLVESGGGLV |
| | | | | QPGGSLKLSCAASGFTFNKYAMNWVRQAPGK |
| | | | | CLEWVARIRSKYNNYATYYADSVKDRFTISRD |
| | | | | DSKNTAYLQMNNLKTEDTAVYYCVRHGNFGN |
| | | | | SYISYWAYWGQGTLVTVSSGGGGSGGGGSGG |
| | | | | GGSQTVVTQEPSLTVSPGGTVTLTCGSSTGAVT |
| | | | | SGNYPNWVQQKPGQAPRGLIGGTKFLAPGTPA |
| | | | | RFSGSLLGGKAALTLSGVQPEDEAEYYCVLWY |
| | | | | SNRWVFGCGTKLTVLGGGGSGGGGSGGGGSD |
| | | | | KTHTCPPCPAPELLGGPSVFLFPPKPKDTLMISR |
| | | | | TPEVTCVVVDVSHEDPEVKFNWYVDGVEVHN |
| | | | | AKTKPCEEQYGSTYRCVSVLTVLHQDWLNGK |
| | | | | EYKCKVSNKALPAPIEKTISKAKGQPREPQVYT |
| | | | | LPPSREEMTKNQVSLTCLVKGFYPSDIAVEWES |
| | | | | NGQPENNYKTTPPVLDSDGSFFLYSKLTVDKS |
| | | | | RWQQGNVFSCSVMHEALHNHYTQKSLSLSPG |
| | | | | KGGGGSGGGGSGGGGSGGGGSGGGGSGGGGS |
| | | | | DKTHTCPPCPAPELLGGPSVFLFPPKPKDTLMIS |
| | | | | RTPEVTCVVVDVSHEDPEVKFNWYVDGVEVH |
| | | | | NAKTKPCEEQYGSTYRCVSVLTVLHQDWLNG |
| | | | | KEYKCKVSNKALPAPIEKTISKAKGQPREPQVY |
| | | | | TLPPSREEMTKNQVSLTCLVKGFYPSDIAVEWE |
| | | | | SNGQPENNYKTTPPVLDSDGSFFLYSKLTVDKS |
| | | | | RWQQGNVFSCSVMHEALHNHYTQKSLSLSPG |
| | | | | KGGGGSGGGGSGGGGSQVQLVQSGAEVKKPG |
| | | | | ASVKVSCKASGYTFTNYWMNWVRQAPGQCL |
| | | | | EWMGNIAYGVKGTNYNQKFQGRVTMTVDTSS |
| | | | | STAYMELSRLRSDDTAVYYCATRYFYVMDYW |
| | | | | GQGTLVTVSSGGGGSGGGGSGGGGSDIQMTQS |
| | | | | PSSLSASVGDRVTITCRASQDISNYLNWYQQKP |
| | | | | GKVPKLLIYYTSRLHSGVPSRFSGSGSGTDFTL |
| | | | | TISSLQPEDVATYYCVQYAQFPLTFGCGTKVEI |
| | | | | KSGGGGSEVQLVESGGGLVQPGGSLKLSCAAS |
| | | | | GFTFNKYAMNWVRQAPGKCLEWVARIRSKYN |
| | | | | NYATYYADSVKDRFTISRDDSKNTAYLQMNN |
| | | | | LKTEDTAVYYCVRHGNFGNSYISYWAYWGQG |
| | | | | TLVTVSSGGGGSGGGGSGGGGSQTVVTQEPSL |
| | | | | TVSPGGTVTLTCGSSTGAVTSGNYPNWVQQKP |
| | | | | GQAPRGLIGGTKFLAPGTPARFSGSLLGGKAAL |
| | | | | TLSGVQPEDEAEYYCVLWYSNRWVFGCGTKL |
| | | | | TVL |
| 316. | MS 25-E3 CCx | artificial | aa | QVQLQESGPGLVKPSETLSLTCTVSGGSISSSSY |
| | 6H10.09x (G4)x | | | FWVWIRQPPGKCLEWIGSIYYSGSTYYNPSLKS |
| | scFcx (G4)x CH3 | | | RVTISVDTSKNQFSLKLNSVTAADTAVYYCAR |
| | 22-A12 CCx | | | LPRGDRMTFDIWGQGTMVTVSSGGGGSGGGG |
| | 6H10.09 - Full | | | SGGGGSEIVLTQSPGTLSLSPGERATLSCRASQS |
| | Sequence | | | VSSSYLAWYQQKPGQAPRLLIYGASSRATGIPD |
| | | | | RFSGSGSGTDFTLTISRLEPEDFAVYYCQQYGS |
| | | | | SPFTFGCGTKLEIKSGGGGSEVQLVESGGGLVQ |
| | | | | PGGSLKLSCAASGFTFNKYAMNWVRQAPGKG |
| | | | | MEWVARIRSKYNNYATYYADAVKDRFTISRD |
| | | | | DSKNTLYLQMNNLKTEDTAVYYCVRAGNFGS |
| | | | | SYISYFAYWGQGTLVTVSSGGGGSGGGGSGGG |
| | | | | GSQTVVTQEPSLTVSPGGTVTITCGSSTGAVTS |
| | | | | GNYPNWIQKKPGQAPRGLIGGTKFLAPGTPAR |
| | | | | FSGSLEGGKAALTLSGVQPEDEAEYYCVLYYS |
| | | | | NRWVFGSGTKLTVLGGGGDKTHTCPPCPAPEL |
| | | | | LGGPSVFLFPPKPKDTLMISRTPEVTCVVVDVS |
| | | | | HEDPEVKFNWYVDGVEVHNAKTKPCEEQYGS |
| | | | | TYRCVSVLTVLHQDWLNGKEYKCKVSNKALP |
| | | | | APIEKTISKAKGQPREPQVYTLPPSREEMTKNQ |
| | | | | VSLTCLVKGFYPSDIAVEWESNGQPENNYKTT |
| | | | | PPVLDSDGSFFLYSKLTVDKSRWQQGNVFSCS |
| | | | | VMHEALHNHYTQKSLSLSPGKGGGGSGGGGS |
| | | | | GGGGSGGGGSGGGGSGGGGSDKTHTCPPCPAP |
| | | | | ELLGGPSVFLFPPKPKDTLMISRTPEVTCVVVD |
| | | | | VSHEDPEVKFNWYVDGVEVHNAKTKPCEEQY |
| | | | | GSTYRCVSVLTVLHQDWLNGKEYKCKVSNKA |
| | | | | LPAPIEKTISKAKGQPREPQVYTLPPSREEMTK |
| | | | | NQVSLTCLVKGFYPSDIAVEWESNGQPENNYK |
| | | | | TTPPVLDSDGSFFLYSKLTVDKSRWQQGNVFS |
| | | | | CSVMHEALHNHYTQKSLSLSPGKGGGGQVQL |
| | | | | VQSGAEVVKPGASVKVSCKASGYTFTSSWMN |
| | | | | WVRQAPGQCLEWMGRIYTGTGETKYSGKFQG |
| | | | | RVTITRDTSASTAYMELSSLTSEDTAVYYCARQ |
| | | | | RDYGALYAMDYWGQGTLVTVSSGGGGSGGG |
| | | | | GSGGGGSDIQLTQSPSFLSASVGDRVTITCRAS |
| | | | | DDIYSYLAWYQQKPGKAPKLLVYNAKTLAEG |
| | | | | VPSRFSGSGSGTEFTLTISSLQPEDFATYYCQNH |
| | | | | DRTPFTFGCGTKVDIKSGGGGSEVQLVESGGG |
| | | | | LVQPGGSLKLSCAASGFTFNKYAMNWVRQAP |
| | | | | GKGMEWVARIRSKYNNYATYYADAVKDRFTI |
| | | | | SRDDSKNTLYLQMNNLKTEDTAVYYCVRAGN |
| | | | | FGSSYISYFAYWGQGTLVTVSSGGGGSGGGGS |
| | | | | GGGGSQTVVTQEPSLTVSPGGTVTITCGSSTGA |
| | | | | VTSGNYPNWIQKKPGQAPRGLIGGTKFLAPGT |
| | | | | PARFSGSLEGGKAALTLSGVQPEDEAEYYCVL |
| | | | | YYSNRWVFGSGTKLTVL |
| 317. | MS 25-E3 CCx | artificial | aa | QVQLQESGPGLVKPSETLSLTCTVSGGSISSSSY |
| | 6H10.09x | | | FWVWIRQPPGKCLEWIGSIYYSGSTYYNPSLKS |
| | (G4S)3x scFcx | | | RVTISVDTSKNQFSLKLNSVTAADTAVYYCAR |
| | (G4S)3x CH3 22- | | | LPRGDRMTFDIWGQGTMVTVSSGGGGSGGGG |
| | A12 CCx 6H10.09 - | | | SGGGGSEIVLTQSPGTLSLSPGERATLSCRASQS |
| | Full Sequence | | | VSSSYLAWYQQKPGQAPRLLIYGASSRATGIPD |
| | | | | RFSGSGSGTDFTLTISRLEPEDFAVYYCQQYGS |
| | | | | SPFTFGCGTKLEIKSGGGGSEVQLVESGGGLVQ |
| | | | | PGGSLKLSCAASGFTFNKYAMNWVRQAPGKG |
| | | | | MEWVARIRSKYNNYATYYADAVKDRFTISRD |
| | | | | DSKNTLYLQMNNLKTEDTAVYYCVRAGNFGS |
| | | | | SYISYFAYWGQGTLVTVSSGGGGSGGGGSGGG |
| | | | | GSQTVVTQEPSLTVSPGGTVTITCGSSTGAVTS |
| | | | | GNYPNWIQKKPGQAPRGLIGGTKFLAPGTPAR |
| | | | | FSGSLEGGKAALTLSGVQPEDEAEYYCVLYYS |
| | | | | NRWVFGSGTKLTVLGGGGSGGGGSGGGGSDK |
| | | | | THTCPPCPAPELLGGPSVFLFPPKPKDTLMISRT |
| | | | | PEVTCVVVDVSHEDPEVKFNWYVDGVEVHNA |
| | | | | KTKPCEEQYGSTYRCVSVLTVLHQDWLNGKE |
| | | | | YKCKVSNKALPAPIEKTISKAKGQPREPQVYTL |
| | | | | PPSREEMTKNQVSLTCLVKGFYPSDIAVEWES |
| | | | | NGQPENNYKTTPPVLDSDGSFFLYSKLTVDKS |
| | | | | RWQQGNVFSCSVMHEALHNHYTQKSLSLSPG |
| | | | | KGGGGSGGGGSGGGGSGGGGSGGGGSGGGGS |
| | | | | DKTHTCPPCPAPELLGGPSVFLFPPKPKDTLMIS |
| | | | | RTPEVTCVVVDVSHEDPEVKFNWYVDGVEVH |
| | | | | NAKTKPCEEQYGSTYRCVSVLTVLHQDWLNG |
| | | | | KEYKCKVSNKALPAPIEKTISKAKGQPREPQVY |
| | | | | TLPPSREEMTKNQVSLTCLVKGFYPSDIAVEWE |
| | | | | SNGQPENNYKTTPPVLDSDGSFFLYSKLTVDKS |
| | | | | RWQQGNVFSCSVMHEALHNHYTQKSLSLSPG |
| | | | | KGGGGSGGGGSGGGGSQVQLVQSGAEVVKPG |
| | | | | ASVKVSCKASGYTFTSSWMNWVRQAPGQCLE |
| | | | | WMGRIYTGTGETKYSGKFQGRVTITRDTSAST |
| | | | | AYMELSSLTSEDTAVYYCARQRDYGALYAMD |
| | | | | YWGQGTLVTVSSGGGGSGGGGSGGGGSDIQL |
| | | | | TQSPSFLSASVGDRVTITCRASDDIYSYLAWYQ |
| | | | | QKPGKAPKLLVYNAKTLAEGVPSRFSGSGSGT |
| | | | | EFTLTISSLQPEDFATYYCQNHDRTPFTFGCGT |
| | | | | KVDIKSGGGGSEVQLVESGGGLVQPGGSLKLS |
| | | | | CAASGFTFNKYAMNWVRQAPGKGMEWVARI |
| | | | | RSKYNNYATYYADAVKDRFTISRDDSKNTLYL |
| | | | | QMNNLKTEDTAVYYCVRAGNFGSSYISYFAY |
| | | | | WGQGTLVTVSSGGGGSGGGGSGGGGSQTVVT |
| | | | | QEPSLTVSPGGTVTITCGSSTGAVTSGNYPNWI |
| | | | | QKKPGQAPRGLIGGTKFLAPGTPARFSGSLEGG |
| | | | | KAALTLSGVQPEDEAEYYCVLYYSNRWVFGS |
| | | | | GTKLTVL |
| 318. | MS 46-A3 CC x | artificial | aa | QVQLVESGGGVVQPGRSLRLSCAASGFTFSSY |
| | I2Ccc(44/100)x | | | GMGWVRQAPGKCLEWVAVISYHGSNKYYAD |
| | (G4S)3x scFcx | | | AVKGRFTISRDNSKNTLYLQMNSLRAEDTAVY |
| | (G4S)3x CH3 | | | YCAREGAHFGSGSYYPLYYYYAMDVWGQGT |
| | 005-D5 CCx | | | TVTVSSGGGGSGGGGSGGGGSEIVTQSPGTLSL |
| | I2Ccc(44/100) - | | | SPGERATLSCRASQSVSSSYLAWYQQKPGQAP |
| | Full Sequence | | | RLLIYGASIRATGIPDRFSGSGSGTDFTLTISRLE |
| | | | | PEDFAVYYCQQTGSSPIFTFGCGTKVEIKSGGG |
| | | | | GSEVQLVESGGGLVQPGGSLKLSCAASGFTFN |
| | | | | KYAMNWVRQAPGKCLEWVARIRSKYNNYAT |
| | | | | YYADSVKDRFTISRDDSKNTAYLQMNNLKTED |
| | | | | TAVYYCVRHGNFGNSYISYWAYWGQGTLVTV |
| | | | | SSGGGGSGGGGSGGGGSQTVVTQEPSLTVSPG |
| | | | | GTVTLTCGSSTGAVTSGNYPNWVQQKPGQAP |
| | | | | RGLIGGTKFLAPGTPARFSGSLLGGKAALTLSG |
| | | | | VQPEDEAEYYCVLWYSNRWVFGCGTKLTVLG |
| | | | | GGGSGGGGSGGGGSDKTHTCPPCPAPELLGGP |
| | | | | SVFLFPPKPKDTLMISRTPEVTCVVVDVSHEDP |
| | | | | EVKFNWYVDGVEVHNAKTKPCEEQYGSTYRC |
| | | | | VSVLTVLHQDWLNGKEYKCKVSNKALPAPIEK |
| | | | | TISKAKGQPREPQVYTLPPSREEMTKNQVSLTC |
| | | | | LVKGFYPSDIAVEWESNGQPENNYKTTPPVLD |
| | | | | SDGSFFLYSKLTVDKSRWQQGNVFSCSVMHEA |
| | | | | LHNHYTQKSLSLSPGKGGGGSGGGGSGGGGS |
| | | | | GGGGSGGGGSGGGGSDKTHTCPPCPAPELLGG |
| | | | | PSVFLFPPKPKDTLMISRTPEVTCVVVDVSHED |
| | | | | PEVKFNWYVDGVEVHNAKTKPCEEQYGSTYR |
| | | | | CVSVLTVLHQDWLNGKEYKCKVSNKALPAPIE |
| | | | | KTISKAKGQPREPQVYTLPPSREEMTKNQVSLT |
| | | | | CLVKGFYPSDIAVEWESNGQPENNYKTTPPVL |
| | | | | DSDGSFFLYSKLTVDKSRWQQGNVFSCSVMHE |
| | | | | ALHNHYTQKSLSLSPGKGGGGSGGGGSGGGG |
| | | | | SEVQLLESGGGLVQPGGSLRLSCAASGFSFSSY |
| | | | | PINWVRQAPGKCLEWVGVIWTGGGTNYASSV |
| | | | | KGRFTISRDNSKNTVYLQMNSLRAEDTAVYYC |
| | | | | AKSRGVYDFKGRGAMDYWGQGTLVTVSSGG |
| | | | | GGSGGGGSGGGGSDIVMTQSPDSLAVSLGERA |
| | | | | TINCKSSQSLLYSSNQKNYFAWYQQKPGQPPK |
| | | | | LLIYWASTRESGVPDRFSGSGSGTDFTLTISSLQ |
| | | | | AEDVAVYYCQQYYSYPYTFGCGTKLEIKSGGG |
| | | | | GSEVQLVESGGGLVQPGGSLKLSCAASGFTFN |
| | | | | KYAMNWVRQAPGKCLEWVARIRSKYNNYAT |
| | | | | YYADSVKDRFTISRDDSKNTAYLQMNNLKTED |
| | | | | TAVYYCVRHGNFGNSYISYWAYWGQGTLVTV |
| | | | | SSGGGGSGGGGSGGGGSQTVVTQEPSLTVSPG |
| | | | | GTVTLTCGSSTGAVTSGNYPNWVQQKPGQAP |
| | | | | RGLIGGTKFLAPGTPARFSGSLLGGKAALTLSG |
| | | | | VQPEDEAEYYCVLWYSNRWVFGCGTKLTVL |
| 319. | MS R4L CC x | artificial | aa | QVQLVQSGAEVKKPGASVKVSCKASGYTFTG |
| | I2C 44/100cc x | | | YYIHWVRQAPGQCLEWMGWINPNSGGTNYA |
| | scFc x CH3 | | | QKFQGRVTMTRDTSISTAYMELSRLRSDDTAV |
| | R170R CC x | | | YYCARVEAVAGREYYYFSGMDVWGQGTTVT |
| | I2C4/100cc0 - | | | VSSGGGGSGGGGSGGGGSSYELTQPPSVSVSP |
| | Full Sequence | | | GQTASITCSGEKLGDKYVYWYQQKPGQSPVL |
| | | | | VIYQSTKRPSGVPERFSGSNSGNTATLTISGTQA |
| | | | | MDEADYYCQAYHASTAVFGCGTKLTVLSGGG |
| | | | | GSEVQLVESGGGLVQPGGSLKLSCAASGFTFN |
| | | | | KYAMNWVRQAPGKCLEWVARIRSKYNNYAT |
| | | | | YYADSVKDRFTISRDDSKNTAYLQMNNLKTED |
| | | | | TAVYYCVRHGNFGNSYISYWAYWGQGTLVTV |
| | | | | SSGGGGSGGGGSGGGGSQTVVTQEPSLTVSPG |
| | | | | GTVTLTCGSSTGAVTSGNYPNWVQQKPGQAP |
| | | | | RGLIGGTKFLAPGTPARFSGSLLGGKAALTLSG |
| | | | | VQPEDEAEYYCVLWYSNRWVFGCGTKLTVLG |
| | | | | GGGSGGGGSGGGGSDKTHTCPPCPAPELLGGP |
| | | | | SVFLFPPKPKDTLMISRTPEVTCVVVDVSHEDP |
| | | | | EVKFNWYVDGVEVHNAKTKPCEEQYGSTYRC |
| | | | | VSVLTVLHQDWLNGKEYKCKVSNKALPAPIEK |
| | | | | TISKAKGQPREPQVYTLPPSREEMTKNQVSLTC |
| | | | | LVKGFYPSDIAVEWESNGQPENNYKTTPPVLD |
| | | | | SDGSFFLYSKLTVDKSRWQQGNVFSCSVMHEA |
| | | | | LHNHYTQKSLSLSPGKGGGGSGGGGSGGGGS |
| | | | | GGGGSGGGGSGGGGSDKTHTCPPCPAPELLGG |
| | | | | PSVFLFPPKPKDTLMISRTPEVTCVVVDVSHED |
| | | | | PEVKFNWYVDGVEVHNAKTKPCEEQYGSTYR |
| | | | | CVSVLTVLHQDWLNGKEYKCKVSNKALPAPIE |
| | | | | KTISKAKGQPREPQVYTLPPSREEMTKNQVSLT |
| | | | | CLVKGFYPSDIAVEWESNGQPENNYKTTPPVL |
| | | | | DSDGSFFLYSKLTVDKSRWQQGNVFSCSVMHE |
| | | | | ALHNHYTQKSLSLSPGKGGGGSGGGGSGGGG |
| | | | | SEVQLLESGGGLVQPGGSLRLSCAASGFTFSSY |
| | | | | WMHWVRQTPGKCLEWVSKIDPSDDYTNYNQ |
| | | | | KVKGRFTISIDKSKNTLYLQMNSLRAEDTAVY |
| | | | | YCARWDYSHFDVWGQGTTVTVSSGGGGSGG |
| | | | | GGSGGGGSEIVMTQSPATLSVSPGERATLTCRA |
| | | | | SSSVSYMHWYQQKPGQAPRLLIYGTSNLVSGV |
| | | | | PARFSGSGSGTEFTLTISSLQSEDFAVYYCQQW |
| | | | | SSYPLTFGCGTKVEIKSGGGGSEVQLVESGGGL |
| | | | | VQPGGSLKLSCAASGFTFNKYAMNWVRQAPG |
| | | | | KCLEWVARIRSKYNNYATYYADSVKDRFTISR |
| | | | | DDSKNTAYLQMNNLKTEDTAVYYCVRHGNFG |
| | | | | NSYISYWAYWGQGTLVTVSSGGGGSGGGGSG |
| | | | | GGGSQTVVTQEPSLTVSPGGTVTLTCGSSTGA |
| | | | | VTSGNYPNWVQQKPGQAPRGLIGGTKFLAPGT |
| | | | | PARFSGSLLGGKAALTLSGVQPEDEAEYYCVL |
| | | | | WYSNRWVFGCGTKLTVL |
| 320. | MS R4L CC x | artificial | aa | QVQLVQSGAEVKKPGASVKVSCKASGYTFTG |
| | I2Ccc(44/100)x | | | YYIHWVRQAPGQCLEWMGWINPNSGGTNYA |
| | (G4S)3x scFcx | | | QKFQGRVTMTRDTSISTAYMELSRLRSDDTAV |
| | (G4S)3x CH3 08- | | | YYCARVEAVAGREYYYFSGMDVWGQGTTVT |
| | A11 CCx | | | VSSGGGGSGGGGSGGGGSSYELTQPPSVSVSP |
| | I2Ccc(44/100) - | | | GQTASITCSGEKLGDKYVYWYQQKPGQSPVL |
| | Full Sequence | | | VIYQSTKRPSGVPERFSGSNSGNTATLTISGTQA |
| | | | | MDEADYYCQAYHASTAVFGCGTKLTVLSGGG |
| | | | | GSEVQLVESGGGLVQPGGSLKLSCAASGFTFN |
| | | | | KYAMNWVRQAPGKCLEWVARIRSKYNNYAT |
| | | | | YYADSVKDRFTISRDDSKNTAYLQMNNLKTED |
| | | | | TAVYYCVRHGNFGNSYISYWAYWGQGTLVTV |
| | | | | SSGGGGSGGGGSGGGGSQTVVTQEPSLTVSPG |
| | | | | GTVTLTCGSSTGAVTSGNYPNWVQQKPGQAP |
| | | | | RGLIGGTKFLAPGTPARFSGSLLGGKAALTLSG |
| | | | | VQPEDEAEYYCVLWYSNRWVFGCGTKLTVLG |
| | | | | GGGSGGGGSGGGGSDKTHTCPPCPAPELLGGP |
| | | | | SVFLFPPKPKDTLMISRTPEVTCVVVDVSHEDP |
| | | | | EVKFNWYVDGVEVHNAKTKPCEEQYGSTYRC |
| | | | | VSVLTVLHQDWLNGKEYKCKVSNKALPAPIEK |
| | | | | TISKAKGQPREPQVYTLPPSREEMTKNQVSLTC |
| | | | | LVKGFYPSDIAVEWESNGQPENNYKTTPPVLD |
| | | | | SDGSFFLYSKLTVDKSRWQQGNVFSCSVMHEA |
| | | | | LHNHYTQKSLSLSPGKGGGGSGGGGSGGGGS |
| | | | | GGGGSGGGGSGGGGSDKTHTCPPCPAPELLGG |
| | | | | PSVFLFPPKPKDTLMISRTPEVTCVVVDVSHED |
| | | | | PEVKFNWYVDGVEVHNAKTKPCEEQYGSTYR |
| | | | | CVSVLTVLHQDWLNGKEYKCKVSNKALPAPIE |
| | | | | KTISKAKGQPREPQVYTLPPSREEMTKNQVSLT |
| | | | | CLVKGFYPSDIAVEWESNGQPENNYKTTPPVL |
| | | | | DSDGSFFLYSKLTVDKSRWQQGNVFSCSVMHE |
| | | | | ALHNHYTQKSLSLSPGKGGGGSGGGGSGGGG |
| | | | | SEVQLLESGGGLVQPGGSLRLSCAASGFTFSSY |
| | | | | WMHWVRQTPGKCLEWVSKIDPSDDYTNYNQ |
| | | | | KVKGRFTISIDKSKNTLYLQMNSLRAEDTAVY |
| | | | | YCARWDYNYFDVWGQGTTVTVSSGGGGSGG |
| | | | | GGSGGGGSEIVMTQSPATLSVSPGERATLTCRA |
| | | | | SSSVSYMHWYQQKPGQAPRLLIYGTSNLVSGV |
| | | | | PARFSGSGSGTEFTLTISSLQSEDFAVYYCQQW |
| | | | | SSYPLTFGCGTKVEIKSGGGGSEVQLVESGGGL |
| | | | | VQPGGSLKLSCAASGFTFNKYAMNWVRQAPG |
| | | | | KCLEWVARIRSKYNNYATYYADSVKDRFTISR |
| | | | | DDSKNTAYLQMNNLKTEDTAVYYCVRHGNFG |
| | | | | NSYISYWAYWGQGTLVTVSSGGGGSGGGGSG |
| | | | | GGGSQTVVTQEPSLTVSPGGTVTLTCGSSTGA |
| | | | | VTSGNYPNWVQQKPGQAPRGLIGGTKFLAPGT |
| | | | | PARFSGSLLGGKAALTLSGVQPEDEAEYYCVL |
| | | | | WYSNRWVFGCGTKLTVL |
| 321. | MS R4L CC x | artificial | Aa | QVQLVQSGAEVKKPGASVKVSCKASGYTFTG |
| | I2Ccc(44/100)x | | | YYIHWVRQAPGQCLEWMGWINPNSGGTNYA |
| | (G4S)3x scFcx | | | QKFQGRVTMTRDTSISTAYMELSRLRSDDTAV |
| | (G4S)3x CH3 | | | YYCARVEAVAGREYYYFSGMDVWGQGTTVT |
| | R164L CCx | | | VSSGGGGSGGGGSGGGGSSYELTQPPSVSVSP |
| | I2Ccc(44/100) - | | | GQTASITCSGEKLGDKYVYWYQQKPGQSPVL |
| | Full Sequence | | | VIYQSTKRPSGVPERFSGSNSGNTATLTISGTQA |
| | | | | MDEADYYCQAYHASTAVFGCGTKLTVLSGGG |
| | | | | GSEVQLVESGGGLVQPGGSLKLSCAASGFTFN |
| | | | | KYAMNWVRQAPGKCLEWVARIRSKYNNYAT |
| | | | | YYADSVKDRFTISRDDSKNTAYLQMNNLKTED |
| | | | | TAVYYCVRHGNFGNSYISYWAYWGQGTLVTV |
| | | | | SSGGGGSGGGGSGGGGSQTVVTQEPSLTVSPG |
| | | | | GTVTLTCGSSTGAVTSGNYPNWVQQKPGQAP |
| | | | | RGLIGGTKFLAPGTPARFSGSLLGGKAALTLSG |
| | | | | VQPEDEAEYYCVLWYSNRWVFGCGTKLTVLG |
| | | | | GGGSGGGGSGGGGSDKTHTCPPCPAPELLGGP |
| | | | | SVFLFPPKPKDTLMISRTPEVTCVVVDVSHEDP |
| | | | | EVKFNWYVDGVEVHNAKTKPCEEQYGSTYRC |
| | | | | VSVLTVLHQDWLNGKEYKCKVSNKALPAPIEK |
| | | | | TISKAKGQPREPQVYTLPPSREEMTKNQVSLTC |
| | | | | LVKGFYPSDIAVEWESNGQPENNYKTTPPVLD |
| | | | | SDGSFFLYSKLTVDKSRWQQGNVFSCSVMHEA |
| | | | | LHNHYTQKSLSLSPGKGGGGSGGGGSGGGGS |
| | | | | GGGGSGGGGSGGGGSDKTHTCPPCPAPELLGG |
| | | | | PSVFLFPPKPKDTLMISRTPEVTCVVVDVSHED |
| | | | | PEVKFNWYVDGVEVHNAKTKPCEEQYGSTYR |
| | | | | CVSVLTVLHQDWLNGKEYKCKVSNKALPAPIE |
| | | | | KTISKAKGQPREPQVYTLPPSREEMTKNQVSLT |
| | | | | CLVKGFYPSDIAVEWESNGQPENNYKTTPPVL |
| | | | | DSDGSFFLYSKLTVDKSRWQQGNVFSCSVMHE |
| | | | | ALHNHYTQKSLSLSPGKGGGGSGGGGSGGGG |
| | | | | SEVQLLESGGGLVQPGGSVRLSCAASGFTFSSY |
| | | | | WMYWVRQAPGKCLEWVSKIDPSDDYTNYNQ |
| | | | | KVKGRFTISIDNSKNTLYLQMNSLRAEDSAVY |
| | | | | YCARWDYTHFDVWGQGTTVTVSSGGGGSGG |
| | | | | GGSGGGGSEIVMTQSPATLSVSPGERATLSCRA |
| | | | | SSSVSYMHWYQQKPGQAPRLLIYGTSNLASGV |
| | | | | PVRFSGSGSGTEFTLTISRLQSEDVAVYYCQQW |
| | | | | SSYPLTFGCGTKVEIKSGGGGSEVQLVESGGGL |
| | | | | VQPGGSLKLSCAASGFTFNKYAMNWVRQAPG |
| | | | | KCLEWVARIRSKYNNYATYYADSVKDRFTISR |
| | | | | DDSKNTAYLQMNNLKTEDTAVYYCVRHGNFG |
| | | | | NSYISYWAYWGQGTLVTVSSGGGGSGGGGSG |
| | | | | GGGSQTVVTQEPSLTVSPGGTVTLTCGSSTGA |
| | | | | VTSGNYPNWVQQKPGQAPRGLIGGTKFLAPGT |
| | | | | PARFSGSLLGGKAALTLSGVQPEDEAEYYCVL |
| | | | | WYSNRWVFGCGTKLTVL |
| 322. | MS R4L CCx | artificial | Aa | QVQLVQSGAEVKKPGASVKVSCKASGYTFTG |
| | I2Ccc(44/100)x | | | YYIHWVRQAPGQCLEWMGWINPNSGGTNYA |
| | (G4)x scFc x (G4) | | | QKFQGRVTMTRDTSISTAYMELSRLRSDDTAV |
| | x CH3 08-A11 | | | YYCARVEAVAGREYYYFSGMDVWGQGTTVT |
| | CCx | | | VSSGGGGSGGGGSGGGGSSYELTQPPSVSVSP |
| | I2Ccc(44/100) - | | | GQTASITCSGEKLGDKYVYWYQQKPGQSPVL |
| | Full Sequence | | | VIYQSTKRPSGVPERFSGSNSGNTATLTISGTQA |
| | | | | MDEADYYCQAYHASTAVFGCGTKLTVLSGGG |
| | | | | GSEVQLVESGGGLVQPGGSLKLSCAASGFTFN |
| | | | | KYAMNWVRQAPGKCLEWVARIRSKYNNYAT |
| | | | | YYADSVKDRFTISRDDSKNTAYLQMNNLKTED |
| | | | | TAVYYCVRHGNFGNSYISYWAYWGQGTLVTV |
| | | | | SSGGGGSGGGGSGGGGSQTVVTQEPSLTVSPG |
| | | | | GTVTLTCGSSTGAVTSGNYPNWVQQKPGQAP |
| | | | | RGLIGGTKFLAPGTPARFSGSLLGGKAALTLSG |
| | | | | VQPEDEAEYYCVLWYSNRWVFGCGTKLTVLG |
| | | | | GGGDKTHTCPPCPAPELLGGPSVFLFPPKPKDT |
| | | | | LMISRTPEVTCVVVDVSHEDPEVKFNWYVDG |
| | | | | VEVHNAKTKPCEEQYGSTYRCVSVLTVLHQD |
| | | | | WLNGKEYKCKVSNKALPAPIEKTISKAKGQPR |
| | | | | EPQVYTLPPSREEMTKNQVSLTCLVKGFYPSDI |
| | | | | AVEWESNGQPENNYKTTPPVLDSDGSFFLYSK |
| | | | | LTVDKSRWQQGNVFSCSVMHEALHNHYTQKS |
| | | | | LSLSPGKGGGGSGGGGSGGGGSGGGGSGGGG |
| | | | | SGGGGSDKTHTCPPCPAPELLGGPSVFLFPPKP |
| | | | | KDTLMISRTPEVTCVVVDVSHEDPEVKFNWYV |
| | | | | DGVEVHNAKTKPCEEQYGSTYRCVSVLTVLH |
| | | | | QDWLNGKEYKCKVSNKALPAPIEKTISKAKGQ |
| | | | | PREPQVYTLPPSREEMTKNQVSLTCLVKGFYPS |
| | | | | DIAVEWESNGQPENNYKTTPPVLDSDGSFFLYS |
| | | | | KLTVDKSRWQQGNVFSCSVMHEALHNHYTQK |
| | | | | SLSLSPGKGGGGEVQLLESGGGLVQPGGSLRLS |
| | | | | CAASGFTFSSYWMHWVRQTPGKCLEWVSKID |
| | | | | PSDDYTNYNQKVKGRFTISIDKSKNTLYLQMN |
| | | | | SLRAEDTAVYYCARWDYNYFDVWGQGTTVT |
| | | | | VSSGGGGSGGGGSGGGGSEIVMTQSPATLSVS |
| | | | | PGERATLTCRASSSVSYMHWYQQKPGQAPRLL |
| | | | | IYGTSNLVSGVPARFSGSGSGTEFTLTISSLQSE |
| | | | | DFAVYYCQQWSSYPLTFGCGTKVEIKSGGGGS |
| | | | | EVQLVESGGGLVQPGGSLKLSCAASGFTFNKY |
| | | | | AMNWVRQAPGKCLEWVARIRSKYNNYATYY |
| | | | | ADSVKDRFTISRDDSKNTAYLQMNNLKTEDTA |
| | | | | VYYCVRHGNFGNSYISYWAYWGQGTLVTVSS |
| | | | | GGGGSGGGGSGGGGSQTVVTQEPSLTVSPGGT |
| | | | | VTLTCGSSTGAVTSGNYPNWVQQKPGQAPRG |
| | | | | LIGGTKFLAPGTPARFSGSLLGGKAALTLSGVQ |
| | | | | PEDEAEYYCVLWYSNRWVFGCGTKLTVL |
| 323. | MS15-B12 CC x | artificial | Aa | QVQLQESGPGLVKPSETLSLTCTVSGGSISSSSY |
| | I2M2 x (G4Q)3x | | | FWGWIRQPPGKCLEWIGNIYYSGSSNYNPSLKS |
| | scFcmod x | | | RVTISVDTSKNQFSLKLSSVTAADTAVYYCAR |
| | (G4Q)3 x CH3 24- | | | LPRGDRDAFDIWGQGTMVTVSSGGGGQGGGG |
| | D7 CC x I2M2 - | | | QGGGGQDIVMTQSPSSLSASVGDRVTITCRASQ |
| | Full Sequence | | | GISNYLAWYQQKPGKVPKLLIYAASTLQSGVP |
| | | | | SRFSGSGSGTDFTLTISSLQPEDFATYYCQQSYS |
| | | | | TPFTFGCGTKVEIKSGGGGQEVQLVESGGGLV |
| | | | | QPGGSLKLSCAASGFTFNKYAINWVREAPGKG |
| | | | | LEWVARIRSKYNNYATYYADAVKDRFTISRDD |
| | | | | SKNTAYLQMNNLKTEDTAVYYCVRNANFGTS |
| | | | | YISYFAYWGQGTLVTVSSGGGGQGGGGQGGG |
| | | | | GQQTVVTQEPSLTVSPGGTVTLTCGSSTGAVTS |
| | | | | GNYPNWVQKKPGQAPRGLIGGTKFLAPGTPAR |
| | | | | FSGSLLGGKAALTLSGVQPEDEAEYYCVLWYS |
| | | | | NRWVFGSGTKLTVLGGGGQGGGGQGGGGQC |
| | | | | PPCPAPELLGGPSVFLFPPKPKDTLMISRTPEVT |
| | | | | CVVVDVSHEEPEVKFNWYVDGVEVHNAKTKP |
| | | | | CEEQYGSTYRCVSVLTVLHQDWLNGKEYKCK |
| | | | | VSNKALPAPIEKTISKAKGQPREPQVYTLPPSRE |
| | | | | EMTKNQVSLTCLVKGFYPSDIAVEWESNGQPE |
| | | | | NNYKTTPPVLDSDGSFFLYSKLTVDKSRWQQG |
| | | | | NVFSCSVMHEALHNHYTQKSLSLSPGKGGGG |
| | | | | QGGGGQGGGGQGGGGQGGGGQGGGGQCPPC |
| | | | | PAPELLGGPSVFLFPPKPKDTLMISRTPEVTCVV |
| | | | | VDVSHEEPEVKFNWYVDGVEVHNAKTKPCEE |
| | | | | QYGSTYRCVSVLTVLHQDWLNGKEYKCKVSN |
| | | | | KALPAPIEKTISKAKGQPREPQVYTLPPSREEM |
| | | | | TKNQVSLTCLVKGFYPSDIAVEWESNGQPENN |
| | | | | YKTTPPVLDSDGSFFLYSKLTVDKSRWQQGNV |
| | | | | FSCSVMHEALHNHYTQKSLSLSPGKGGGGQG |
| | | | | GGGQGGGGQQVQLVQSGAEVKKPGASVKVSC |
| | | | | KASGYTFTNYWMNWVRQAPGQCLEWMGNIH |
| | | | | SKAHGTNYNQKFQGRVTMTVDTSSSTAYMEL |
| | | | | SRLRSDDTAVYYCATRYFYVMDYWGQGTLVT |
| | | | | VSSGGGGQGGGGQGGGGQDIQMTQSPSSLSAS |
| | | | | VGDRVTITCRASQDISNYLNWYQQKPGKVPKL |
| | | | | LIYYTSRLHSGVPSRFSGSGSGTDFTLTISSLQPE |
| | | | | DVATYYCVQYAQFPLTFGCGTKVEIKSGGGGQ |
| | | | | EVQLVESGGGLVQPGGSLKLSCAASGFTFNKY |
| | | | | AINWVREAPGKGLEWVARIRSKYNNYATYYA |
| | | | | DAVKDRFTISRDDSKNTAYLQMNNLKTEDTA |
| | | | | VYYCVRNANFGTSYISYFAYWGQGTLVTVSSG |
| | | | | GGGQGGGGQGGGGQQTVVTQEPSLTVSPGGT |
| | | | | VTLTCGSSTGAVTSGNYPNWVQKKPGQAPRG |
| | | | | LIGGTKFLAPGTPARFSGSLLGGKAALTLSGVQ |
| | | | | PEDEAEYYCVLWYSNRWVFGSGTKLTVL |
| 324. | heFc(A) x(G4)x | artificial | Aa | DKTHTCPPCPAPELLGGPSVFLFPPKPKDTLMIS |
| | MS 15-B12 CCx | | | RTPEVTCVVVDVSHEDPEVKFNWYVDGVEVH |
| | 6H10.09 | | | NAKTKPCEEQYGSTYRCVSVLTVLHQDWLNG |
| | | | | KEYKCKVSNKALPAPIEKTISKAKGQPREPQVY |
| | | | | TLPPSREEMTKNQVSLTCLVKGFYPSDIAVEWE |
| | | | | SNGQPENNYDTTPPVLDSDGSFFLYSDLTVDKS |
| | | | | RWQQGNVFSCSVMHEALHNHYTQDSLSLSPG |
| | | | | KGGGGQVQLQESGPGLVKPSETLSLTCTVSGG |
| | | | | SISSSSYFWGWIRQPPGKCLEWIGNIYYSGSSN |
| | | | | YNPSLKSRVTISVDTSKNQFSLKLSSVTAADTA |
| | | | | VYYCARLPRGDRDAFDIWGQGTMVTVSSGGG |
| | | | | GSGGGGSGGGGSDIVMTQSPSSLSASVGDRVTI |
| | | | | TCRASQGISNYLAWYQQKPGKVPKLLIYAAST |
| | | | | LQSGVPSRFSGSGSGTDFTLTISSLQPEDFATYY |
| | | | | CQQSYSTPFTFGCGTKVEIKSGGGGSEVQLVES |
| | | | | GGGLVQPGGSLKLSCAASGFTFNKYAMNWVR |
| | | | | QAPGKGMEWVARIRSKYNNYATYYADAVKD |
| | | | | RFTISRDDSKNTLYLQMNNLKTEDTAVYYCVR |
| | | | | AGNFGSSYISYFAYWGQGTLVTVSSGGGGSGG |
| | | | | GGSGGGGSQTVVTQEPSLTVSPGGTVTITCGSS |
| | | | | TGAVTSGNYPNWIQKKPGQAPRGLIGGTKFLA |
| | | | | PGTPARFSGSLEGGKAALTLSGVQPEDEAEYY |
| | | | | CVLYYSNRWVFGSGTKLTVL |
| 325. | CH3 15-E11 CC | artificial | Aa | QVQLVQSGAEVKKPGASVKVSCKASGYTFTN |
| | 6H10.09 x (G4)x | | | YWMNWVRQAPGQCLEWMGNIAYGVKGTNY |
| | heFc(B) | | | NQKFQGRVTMTVDTSSSTAYMELSRLRSDDTA |
| | | | | VYYCATRYFYVMDYWGQGTLVTVSSGGGGS |
| | | | | GGGGSGGGGSDIQMTQSPSSLSASVGDRVTITC |
| | | | | RASQDISNYLNWYQQKPGKVPKLLIYYTSRLH |
| | | | | SGVPSRFSGSGSGTDFTLTISSLQPEDVATYYCV |
| | | | | QYAQFPLTFGCGTKVEIKSGGGGSEVQLVESG |
| | | | | GGLVQPGGSLKLSCAASGFTFNKYAMNWVRQ |
| | | | | APGKGMEWVARIRSKYNNYATYYADAVKDR |
| | | | | FTISRDDSKNTLYLQMNNLKTEDTAVYYCVRA |
| | | | | GNFGSSYISYFAYWGQGTLVTVSSGGGGSGGG |
| | | | | GSGGGGSQTVVTQEPSLTVSPGGTVTITCGSST |
| | | | | GAVTSGNYPNWIQKKPGQAPRGLIGGTKFLAP |
| | | | | GTPARFSGSLEGGKAALTLSGVQPEDEAEYYC |
| | | | | VLYYSNRWVFGSGTKLTVLGGGGDKTHTCPP |
| | | | | CPAPELLGGPSVFLFPPKPKDTLMISRTPEVTCV |
| | | | | VVDVSHEDPEVKFNWYVDGVEVHNAKTKPCE |
| | | | | EQYGSTYRCVSVLTVLHQDWLNGKEYKCKVS |
| | | | | NKALPAPIEKTISKAKGQPREPQVYTLPPSRKE |
| | | | | MTKNQVSLTCLVKGFYPSDIAVEWESNGQPEN |
| | | | | NYKTTPPVLKSDGSFFLYSKLTVDKSRWQQGN |
| | | | | VFSCSVMHEALHNHYTQKSLSLSPGK |
| 326. | heFc(B) x (G4)x | artificial | Aa | DKTHTCPPCPAPELLGGPSVFLFPPKPKDTLMIS |
| | CH3 15-E11 CCx | | | RTPEVTCVVVDVSHEDPEVKFNWYVDGVEVH |
| | 6H10.09 | | | NAKTKPCEEQYGSTYRCVSVLTVLHQDWLNG |
| | | | | KEYKCKVSNKALPAPIEKTISKAKGQPREPQVY |
| | | | | TLPPSRKEMTKNQVSLTCLVKGFYPSDIAVEW |
| | | | | ESNGQPENNYKTTPPVLKSDGSFFLYSKLTVDK |
| | | | | SRWQQGNVFSCSVMHEALHNHYTQKSLSLSP |
| | | | | GKGGGGQVQLVQSGAEVKKPGASVKVSCKAS |
| | | | | GYTFTNYWMNWVRQAPGQCLEWMGNIAYGV |
| | | | | KGTNYNQKFQGRVTMTVDTSSSTAYMELSRL |
| | | | | RSDDTAVYYCATRYFYVMDYWGQGTLVTVSS |
| | | | | GGGGSGGGGSGGGGSDIQMTQSPSSLSASVGD |
| | | | | RVTITCRASQDISNYLNWYQQKPGKVPKLLIY |
| | | | | YTSRLHSGVPSRFSGSGSGTDFTLTISSLQPEDV |
| | | | | ATYYCVQYAQFPLTFGCGTKVEIKSGGGGSEV |
| | | | | QLVESGGGLVQPGGSLKLSCAASGFTFNKYAM |
| | | | | NWVRQAPGKGMEWVARIRSKYNNYATYYAD |
| | | | | AVKDRFTISRDDSKNTLYLQMNNLKTEDTAVY |
| | | | | YCVRAGNFGSSYISYFAYWGQGTLVTVSSGGG |
| | | | | GSGGGGSGGGGSQTVVTQEPSLTVSPGGTVTIT |
| | | | | CGSSTGAVTSGNYPNWIQKKPGQAPRGLIGGT |
| | | | | KFLAPGTPARFSGSLEGGKAALTLSGVQPEDEA |
| | | | | EYYCVLYYSNRWVFGSGTKLTVL |
| 327. | MS 15-B12 CCx | artificial | Aa | QVQLQESGPGLVKPSETLSLTCTVSGGSISSSSY |
| | 6H10.09x (G4)x | | | FWGWIRQPPGKCLEWIGNIYYSGSSNYNPSLKS |
| | heFc(A) | | | RVTISVDTSKNQFSLKLSSVTAADTAVYYCAR |
| | | | | LPRGDRDAFDIWGQGTMVTVSSGGGGSGGGG |
| | | | | SGGGGSDIVMTQSPSSLSASVGDRVTITCRASQ |
| | | | | GISNYLAWYQQKPGKVPKLLIYAASTLQSGVP |
| | | | | SRFSGSGSGTDFTLTISSLQPEDFATYYCQQSYS |
| | | | | TPFTFGCGTKVEIKSGGGGSEVQLVESGGGLV |
| | | | | QPGGSLKLSCAASGFTFNKYAMNWVRQAPGK |
| | | | | GMEWVARIRSKYNNYATYYADAVKDRFTISR |
| | | | | DDSKNTLYLQMNNLKTEDTAVYYCVRAGNFG |
| | | | | SSYISYFAYWGQGTLVTVSSGGGGSGGGGSGG |
| | | | | GGSQTVVTQEPSLTVSPGGTVTITCGSSTGAVT |
| | | | | SGNYPNWIQKKPGQAPRGLIGGTKFLAPGTPA |
| | | | | RFSGSLEGGKAALTLSGVQPEDEAEYYCVLYY |
| | | | | SNRWVFGSGTKLTVLGGGGDKTHTCPPCPAPE |
| | | | | LLGGPSVFLFPPKPKDTLMISRTPEVTCVVVDV |
| | | | | SHEDPEVKFNWYVDGVEVHNAKTKPCEEQYG |
| | | | | STYRCVSVLTVLHQDWLNGKEYKCKVSNKAL |
| | | | | PAPIEKTISKAKGQPREPQVYTLPPSREEMTKN |
| | | | | QVSLTCLVKGFYPSDIAVEWESNGQPENNYDT |
| | | | | TPPVLDSDGSFFLYSDLTVDKSRWQQGNVFSC |
| | | | | SVMHEALHNHYTQDSLSLSPGK |
| 328. | H MS 15-B12 x H | artificial | Aa | QVQLQESGPGLVKPSETLSLTCTVSGGSISSSSY |
| | 6H10.09 x (G4S)3 | | | FWGWIRQPPGKCLEWIGNIYYSGSSNYNPSLKS |
| | x heFc(A) x | | | RVTISVDTSKNQFSLKLSSVTAADTAVYYCAR |
| | (G4S)3 x H CH3 | | | LPRGDRDAFDIWGQGTMVTVSSGGGGSGGGG |
| | 15-E11 x H | | | SGGGGSGGGGSEVQLVESGGGLVQPGGSLKLS |
| | 6H10.09 | | | CAASGFTFNKYAMNWVRQAPGKGMEWVARI |
| | | | | RSKYNNYATYYADAVKDRFTISRDDSKNTLYL |
| | | | | QMNNLKTEDTAVYYCVRAGNFGSSYISYFAY |
| | | | | WGQGTLVTVSSGGGGSGGGGSGGGGSDKTHT |
| | | | | CPPCPAPELLGGPSVFLFPPKPKDTLMISRTPEV |
| | | | | TCVVVDVSHEDPEVKFNWYVDGVEVHNAKT |
| | | | | KPCEEQYGSTYRCVSVLTVLHQDWLNGKEYK |
| | | | | CKVSNKALPAPIEKTISKAKGQPREPQVYTLPP |
| | | | | SREEMTKNQVSLTCLVKGFYPSDIAVEWESNG |
| | | | | QPENNYDTTPPVLDSDGSFFLYSDLTVDKSRW |
| | | | | QQGNVFSCSVMHEALHNHYTQDSLSLSPGKG |
| | | | | GGGSGGGGSGGGGSQVQLVQSGAEVKKPGAS |
| | | | | VKVSCKASGYTFTNYWMNWVRQAPGQCLEW |
| | | | | MGNIAYGVKGTNYNQKFQGRVTMTVDTSSST |
| | | | | AYMELSRLRSDDTAVYYCATRYFYVMDYWG |
| | | | | QGTLVTVSSGGGGSGGGGSGGGGSGGGGSEV |
| | | | | QLVESGGGLVQPGGSLKLSCAASGFTFNKYAM |
| | | | | NWVRQAPGKGMEWVARIRSKYNNYATYYAD |
| | | | | AVKDRFTISRDDSKNTLYLQMNNLKTEDTAVY |
| | | | | YCVRAGNFGSSYISYFAYWGQGTLVTVSS |
| 329. | L MS 15-B12 x L | artificial | Aa | DIVMTQSPSSLSASVGDRVTITCRASQGISNYL |
| | 6H10.09 x (G4S)3 | | | AWYQQKPGKVPKLLIYAASTLQSGVPSRFSGS |
| | x heFc(B) x | | | GSGTDFTLTISSLQPEDFATYYCQQSYSTPFTFG |
| | (G4S)3 x L CH3 | | | CGTKVEIKSGGGGSGGGGSGGGGSGGGGSQT |
| | 15-E11 x L | | | VVTQEPSLTVSPGGTVTITCGSSTGAVTSGNYP |
| | 6H10.09 | | | NWIQKKPGQAPRGLIGGTKFLAPGTPARFSGSL |
| | | | | EGGKAALTLSGVQPEDEAEYYCVLYYSNRWV |
| | | | | FGSGTKLTVLGGGGSGGGGSGGGGSDKTHTCP |
| | | | | PCPAPELLGGPSVFLFPPKPKDTLMISRTPEVTC |
| | | | | VVVDVSHEDPEVKFNWYVDGVEVHNAKTKPC |
| | | | | EEQYGSTYRCVSVLTVLHQDWLNGKEYKCKV |
| | | | | SNKALPAPIEKTISKAKGQPREPQVYTLPPSRKE |
| | | | | MTKNQVSLTCLVKGFYPSDIAVEWESNGQPEN |
| | | | | NYKTTPPVLKSDGSFFLYSKLTVDKSRWQQGN |
| | | | | VFSCSVMHEALHNHYTQKSLSLSPGKGGGGSG |
| | | | | GGGSGGGGSDIQMTQSPSSLSASVGDRVTITCR |
| | | | | ASQDISNYLNWYQQKPGKVPKLLIYYTSRLHS |
| | | | | GVPSRFSGSGSGTDFTLTISSLQPEDVATYYCV |
| | | | | QYAQFPLTFGCGTKVEIKSGGGGSGGGGSGGG |
| | | | | GSGGGGSQTVVTQEPSLTVSPGGTVTITCGSST |
| | | | | GAVTSGNYPNWIQKKPGQAPRGLIGGTKFLAP |
| | | | | GTPARFSGSLEGGKAALTLSGVQPEDEAEYYC |
| | | | | VLYYSNRWVFGSGTKLTVL |
| 330. | IgG1 subtype | artificial | aa | DKTHTCPPCP |
| | hinge | | | |
| 331. | IgG2 subtype | artificial | aa | ERKCCVECPPCP |
| | hinge | | | |
| 332. | IgG3 subtype | artificial | aa | ELKTPLDTTHTCPRCP |
| | hinge | | | |
| 333. | IgG3 subtype | artificial | aa | ELKTPLGDTTHTCPRCP |
| | hinge | | | |
| 334. | EpCAM 5-10 LH - | artificial | aa | NYWLG |
| | HCDR1 | | | |
| 335. | EpCAM 5-10 LH - | artificial | aa | DIFPGSGNIHYNEKFKG |
| | HCDR2 | | | |
| 336. | EpCAM 5-10 LH - | artificial | aa | LRNWDEPMDY |
| | HCDR3 | | | |
| 337. | EpCAM 5-10 LH - | artificial | aa | KSSQSLLNSGNQKNYLT |
| | LCDR1 | | | |
| 338. | EpCAM 5-10 LH - | artificial | Aa | WASTRES |
| | LCDR2 | | | |
| 339. | EpCAM 5-10 LH - | artificial | Aa | QNDYSYPLT |
| | LCDR3 | | | |
| 340. | EpCAM 5-10 LH - | artificial | Aa | EVQLLEQSGAELVRPGTSVKISCKASGYAFTNY |
| | VH | | | WLGWVKQRPGHGLEWIGDIFPGSGNIHYNEKF |
| | | | | KGKATLTADKSSSTAYMQLSSLTFEDSAVYFC |
| | | | | ARLRNWDEPMDYWGQGTTVTVSS |
| 341. | EpCAM 5-10 LH - | artificial | Aa | ELVMTQSPSSLTVTAGEKVTMSCKSSQSLLNS |
| | VL | | | GNQKNYLTWYQQKPGQPPKLLIYWASTRESG |
| | | | | VPDRFTGSGSGTDFTLTISSVQAEDLAVYYCQN |
| | | | | DYSYPLTFGAGTKLEIK |
| 342. | EPCAM 5-10 x | artificial | Aa | ELVMTQSPSSLTVTAGEKVTMSCKSSQSLLNS |
| | scFc x H2 x I2Ccc | | | GNQKNYLTWYQQKPGQPPKLLIYWASTRESG |
| | x I2Ccc - Full | | | VPDRFTGSGSGTDFTLTISSVQAEDLAVYYCQN |
| | Sequence | | | DYSYPLTFGAGTKLEIKGGGGSGGGGSGGGGS |
| | | | | EVQLLEQSGAELVRPGTSVKISCKASGYAFTNY |
| | | | | WLGWVKQRPGHGLEWIGDIFPGSGNIHYNEKF |
| | | | | KGKATLTADKSSSTAYMQLSSLTFEDSAVYFC |
| | | | | ARLRNWDEPMDYWGQGTTVTVSSGGGGDKT |
| | | | | HTCPPCPAPELLGGPSVFLFPPKPKDTLMISRTP |
| | | | | EVTCVVVDVSHEDPEVKFNWYVDGVEVHNA |
| | | | | KTKPCEEQYGSTYRCVSVLTVLHQDWLNGKE |
| | | | | YKCKVSNKALPAPIEKTISKAKGQPREPQVYTL |
| | | | | PPSREEMTKNQVSLTCLVKGFYPSDIAVEWES |
| | | | | NGQPENNYKTTPPVLDSDGSFFLYSKLTVDKS |
| | | | | RWQQGNVFSCSVMHEALHNHYTQKSLSLSPG |
| | | | | KGGGGSGGGGSGGGGSGGGGSGGGGSGGGGS |
| | | | | DKTHTCPPCPAPELLGGPSVFLFPPKPKDTLMIS |
| | | | | RTPEVTCVVVDVSHEDPEVKFNWYVDGVEVH |
| | | | | NAKTKPCEEQYGSTYRCVSVLTVLHQDWLNG |
| | | | | KEYKCKVSNKALPAPIEKTISKAKGQPREPQVY |
| | | | | TLPPSREEMTKNQVSLTCLVKGFYPSDIAVEWE |
| | | | | SNGQPENNYKTTPPVLDSDGSFFLYSKLTVDKS |
| | | | | RWQQGNVFSCSVMHEALHNHYTQKSLSLSPG |
| | | | | KGGGGEVQLLESGGGVVQPGRSLRLSCAASGF |
| | | | | TFSSYGMGWVRQAPGKGLEWVAVISYDGSNK |
| | | | | YYADSVKGRFTISRDNSKNTLYLQMNSLRAED |
| | | | | TAVYYCAREGAHFGSGSYYPLYYYYAMDVW |
| | | | | GQGTTVTVSSGGGGSGGGGSGGGGSELTLTQS |
| | | | | PGTLSLSPGERATLSCRASQSVSSSYLAWYQQK |
| | | | | PGQAPRLLIYGASIRATGIPDRFSGSGSGTDFTL |
| | | | | TISRLEPEDFAVYYCQQYGSSPIFTFGPGTKVEI |
| | | | | KSGGGGSEVQLVESGGGLVQPGGSLKLSCAAS |
| | | | | GFTFNKYAMNWVRQAPGKCLEWVARIRSKYN |
| | | | | NYATYYADSVKDRFTISRDDSKNTAYLQMNN |
| | | | | LKTEDTAVYYCVRHGNFGNSYISYWAYWGQG |
| | | | | TLVTVSSGGGGSGGGGSGGGGSQTVVTQEPSL |
| | | | | TVSPGGTVTLTCGSSTGAVTSGNYPNWVQQKP |
| | | | | GQAPRGLIGGTKFLAPGTPARFSGSLLGGKAAL |
| | | | | TLSGVQPEDEAEYYCVLWYSNRWVFGCGTKL |
| | | | | TVLGGGGSGGGGSGGGGSEVQLVESGGGLVQ |
| | | | | PGGSLKLSCAASGFTFNKYAMNWVRQAPGKC |
| | | | | LEWVARIRSKYNNYATYYADSVKDRFTISRDD |
| | | | | SKNTAYLQMNNLKTEDTAVYYCVRHGNFGNS |
| | | | | YISYWAYWGQGTLVTVSSGGGGSGGGGSGGG |
| | | | | GSQTVVTQEPSLTVSPGGTVTLTCGSSTGAVTS |
| | | | | GNYPNWVQQKPGQAPRGLIGGTKFLAPGTPAR |
| | | | | FSGSLLGGKAALTLSGVQPEDEAEYYCVLWYS |
| | | | | NRWVFGCGTKLTVL |
| 343. | EPCAM 5-10 x | artificial | Aa | ELVMTQSPSSLTVTAGEKVTMSCKSSQSLLNS |
| | H2 x scFc x | | | GNQKNYLTWYQQKPGQPPKLLIYWASTRESG |
| | I2Ccc x I2Ccc - | | | VPDRFTGSGSGTDFTLTISSVQAEDLAVYYCQN |
| | Full Sequence | | | DYSYPLTFGAGTKLEIKGGGGSGGGGSGGGGS |
| | | | | EVQLLEQSGAELVRPGTSVKISCKASGYAFTNY |
| | | | | WLGWVKQRPGHGLEWIGDIFPGSGNIHYNEKF |
| | | | | KGKATLTADKSSSTAYMQLSSLTFEDSAVYFC |
| | | | | ARLRNWDEPMDYWGQGTTVTVSSGGGGSEV |
| | | | | QLLESGGGVVQPGRSLRLSCAASGFTFSSYGM |
| | | | | GWVRQAPGKGLEWVAVISYDGSNKYYADSV |
| | | | | KGRFTISRDNSKNTLYLQMNSLRAEDTAVYYC |
| | | | | AREGAHFGSGSYYPLYYYYAMDVWGQGTTV |
| | | | | TVSSGGGGSGGGGSGGGGSELTLTQSPGTLSLS |
| | | | | PGERATLSCRASQSVSSSYLAWYQQKPGQAPR |
| | | | | LLIYGASIRATGIPDRFSGSGSGTDFTLTISRLEP |
| | | | | EDFAVYYCQQYGSSPIFTFGPGTKVEIKSGGGG |
| | | | | DKTHTCPPCPAPELLGGPSVFLFPPKPKDTLMIS |
| | | | | RTPEVTCVVVDVSHEDPEVKFNWYVDGVEVH |
| | | | | NAKTKPCEEQYGSTYRCVSVLTVLHQDWLNG |
| | | | | KEYKCKVSNKALPAPIEKTISKAKGQPREPQVY |
| | | | | TLPPSREEMTKNQVSLTCLVKGFYPSDIAVEWE |
| | | | | SNGQPENNYKTTPPVLDSDGSFFLYSKLTVDKS |
| | | | | RWQQGNVFSCSVMHEALHNHYTQKSLSLSPG |
| | | | | KGGGGSGGGGSGGGGSGGGGSGGGGSGGGGS |
| | | | | DKTHTCPPCPAPELLGGPSVFLFPPKPKDTLMIS |
| | | | | RTPEVTCVVVDVSHEDPEVKFNWYVDGVEVH |
| | | | | NAKTKPCEEQYGSTYRCVSVLTVLHQDWLNG |
| | | | | KEYKCKVSNKALPAPIEKTISKAKGQPREPQVY |
| | | | | TLPPSREEMTKNQVSLTCLVKGFYPSDIAVEWE |
| | | | | SNGQPENNYKTTPPVLDSDGSFFLYSKLTVDKS |
| | | | | RWQQGNVFSCSVMHEALHNHYTQKSLSLSPG |
| | | | | KGGGGEVQLVESGGGLVQPGGSLKLSCAASGF |
| | | | | TFNKYAMNWVRQAPGKCLEWVARIRSKYNN |
| | | | | YATYYADSVKDRFTISRDDSKNTAYLQMNNL |
| | | | | KTEDTAVYYCVRHGNFGNSYISYWAYWGQGT |
| | | | | LVTVSSGGGGSGGGGSGGGGSQTVVTQEPSLT |
| | | | | VSPGGTVTLTCGSSTGAVTSGNYPNWVQQKPG |
| | | | | QAPRGLIGGTKFLAPGTPARFSGSLLGGKAALT |
| | | | | LSGVQPEDEAEYYCVLWYSNRWVFGCGTKLT |
| | | | | VLGGGGSGGGGSGGGGSEVQLVESGGGLVQP |
| | | | | GGSLKLSCAASGFTFNKYAMNWVRQAPGKCL |
| | | | | EWVARIRSKYNNYATYYADSVKDRFTISRDDS |
| | | | | KNTAYLQMNNLKTEDTAVYYCVRHGNFGNSY |
| | | | | ISYWAYWGQGTLVTVSSGGGGSGGGGSGGGG |
| | | | | SQTVVTQEPSLTVSPGGTVTLTCGSSTGAVTSG |
| | | | | NYPNWVQQKPGQAPRGLIGGTKFLAPGTPARF |
| | | | | SGSLLGGKAALTLSGVQPEDEAEYYCVLWYSN |
| | | | | RWVFGCGTKLTVL |
| 344. | EPCAM 5-10 x | artificial | Aa | ELVMTQSPSSLTVTAGEKVTMSCKSSQSLLNS |
| | H2 x I2Ccc x scFc | | | GNQKNYLTWYQQKPGQPPKLLIYWASTRESG |
| | x I2Ccc - Full | | | VPDRFTGSGSGTDFTLTISSVQAEDLAVYYCQN |
| | Sequence | | | DYSYPLTFGAGTKLEIKGGGGSGGGGSGGGGS |
| | | | | EVQLLEQSGAELVRPGTSVKISCKASGYAFTNY |
| | | | | WLGWVKQRPGHGLEWIGDIFPGSGNIHYNEKF |
| | | | | KGKATLTADKSSSTAYMQLSSLTFEDSAVYFC |
| | | | | ARLRNWDEPMDYWGQGTTVTVSSGGGGSEV |
| | | | | QLLESGGGVVQPGRSLRLSCAASGFTFSSYGM |
| | | | | GWVRQAPGKGLEWVAVISYDGSNKYYADSV |
| | | | | KGRFTISRDNSKNTLYLQMNSLRAEDTAVYYC |
| | | | | AREGAHFGSGSYYPLYYYYAMDVWGQGTTV |
| | | | | TVSSGGGGSGGGGSGGGGSELTLTQSPGTLSLS |
| | | | | PGERATLSCRASQSVSSSYLAWYQQKPGQAPR |
| | | | | LLIYGASIRATGIPDRESGSGSGTDFTLTISRLEP |
| | | | | EDFAVYYCQQYGSSPIFTFGPGTKVEIKSGGGG |
| | | | | SEVQLVESGGGLVQPGGSLKLSCAASGFTENK |
| | | | | YAMNWVRQAPGKCLEWVARIRSKYNNYATY |
| | | | | YADSVKDRFTISRDDSKNTAYLQMNNLKTEDT |
| | | | | AVYYCVRHGNFGNSYISYWAYWGQGTLVTVS |
| | | | | SGGGGSGGGGSGGGGSQTVVTQEPSLTVSPGG |
| | | | | TVTLTCGSSTGAVTSGNYPNWVQQKPGQAPR |
| | | | | GLIGGTKFLAPGTPARFSGSLLGGKAALTLSGV |
| | | | | QPEDEAEYYCVLWYSNRWVFGCGTKLTVLGG |
| | | | | GGDKTHTCPPCPAPELLGGPSVFLFPPKPKDTL |
| | | | | MISRTPEVTCVVVDVSHEDPEVKFNWYVDGV |
| | | | | EVHNAKTKPCEEQYGSTYRCVSVLTVLHQDW |
| | | | | LNGKEYKCKVSNKALPAPIEKTISKAKGQPREP |
| | | | | QVYTLPPSREEMTKNQVSLTCLVKGFYPSDIAV |
| | | | | EWESNGQPENNYKTTPPVLDSDGSFFLYSKLT |
| | | | | VDKSRWQQGNVFSCSVMHEALHNHYTQKSLS |
| | | | | LSPGKGGGGSGGGGSGGGGSGGGGSGGGGSG |
| | | | | GGGSDKTHTCPPCPAPELLGGPSVFLFPPKPKD |
| | | | | TLMISRTPEVTCVVVDVSHEDPEVKFNWYVDG |
| | | | | VEVHNAKTKPCEEQYGSTYRCVSVLTVLHQD |
| | | | | WLNGKEYKCKVSNKALPAPIEKTISKAKGQPR |
| | | | | EPQVYTLPPSREEMTKNQVSLTCLVKGFYPSDI |
| | | | | AVEWESNGQPENNYKTTPPVLDSDGSFFLYSK |
| | | | | LTVDKSRWQQGNVFSCSVMHEALHNHYTQKS |
| | | | | LSLSPGKGGGGEVQLVESGGGLVQPGGSLKLS |
| | | | | CAASGFTFNKYAMNWVRQAPGKCLEWVARIR |
| | | | | SKYNNYATYYADSVKDRFTISRDDSKNTAYLQ |
| | | | | MNNLKTEDTAVYYCVRHGNFGNSYISYWAY |
| | | | | WGQGTLVTVSSGGGGSGGGGSGGGGSQTVVT |
| | | | | QEPSLTVSPGGTVTLTCGSSTGAVTSGNYPNW |
| | | | | VQQKPGQAPRGLIGGTKFLAPGTPARFSGSLLG |
| | | | | GKAALTLSGVQPEDEAEYYCVLWYSNRWVFG |
| | | | | CGTKLTVL |
| 345. | EPCAM 5-10 x | artificial | Aa | ELVMTQSPSSLTVTAGEKVTMSCKSSQSLLNS |
| | H2 x I2Ccc x | | | GNQKNYLTWYQQKPGQPPKLLIYWASTRESG |
| | I2Ccc x scFc - | | | VPDRFTGSGSGTDFTLTISSVQAEDLAVYYCQN |
| | Full Sequence | | | DYSYPLTFGAGTKLEIKGGGGSGGGGSGGGGS |
| | | | | EVQLLEQSGAELVRPGTSVKISCKASGYAFTNY |
| | | | | WLGWVKQRPGHGLEWIGDIFPGSGNIHYNEKF |
| | | | | KGKATLTADKSSSTAYMQLSSLTFEDSAVYFC |
| | | | | ARLRNWDEPMDYWGQGTTVTVSSGGGGSEV |
| | | | | QLLESGGGVVQPGRSLRLSCAASGFTFSSYGM |
| | | | | GWVRQAPGKGLEWVAVISYDGSNKYYADSV |
| | | | | KGRFTISRDNSKNTLYLQMNSLRAEDTAVYYC |
| | | | | AREGAHFGSGSYYPLYYYYAMDVWGQGTTV |
| | | | | TVSSGGGGSGGGGSGGGGSELTLTQSPGTLSLS |
| | | | | PGERATLSCRASQSVSSSYLAWYQQKPGQAPR |
| | | | | LLIYGASIRATGIPDRESGSGSGTDFTLTISRLEP |
| | | | | EDFAVYYCQQYGSSPIFTFGPGTKVEIKSGGGG |
| | | | | SEVQLVESGGGLVQPGGSLKLSCAASGFTFNK |
| | | | | YAMNWVRQAPGKCLEWVARIRSKYNNYATY |
| | | | | YADSVKDRFTISRDDSKNTAYLQMNNLKTEDT |
| | | | | AVYYCVRHGNFGNSYISYWAYWGQGTLVTVS |
| | | | | SGGGGSGGGGSGGGGSQTVVTQEPSLTVSPGG |
| | | | | TVTLTCGSSTGAVTSGNYPNWVQQKPGQAPR |
| | | | | GLIGGTKFLAPGTPARFSGSLLGGKAALTLSGV |
| | | | | QPEDEAEYYCVLWYSNRWVFGCGTKLTVLGG |
| | | | | GGSGGGGSGGGGSEVQLVESGGGLVQPGGSL |
| | | | | KLSCAASGFTFNKYAMNWVRQAPGKCLEWV |
| | | | | ARIRSKYNNYATYYADSVKDRFTISRDDSKNT |
| | | | | AYLQMNNLKTEDTAVYYCVRHGNFGNSYISY |
| | | | | WAYWGQGTLVTVSSGGGGSGGGGSGGGGSQ |
| | | | | TVVTQEPSLTVSPGGTVTLTCGSSTGAVTSGNY |
| | | | | PNWVQQKPGQAPRGLIGGTKFLAPGTPARFSG |
| | | | | SLLGGKAALTLSGVQPEDEAEYYCVLWYSNR |
| | | | | WVFGCGTKLTVLGGGGDKTHTCPPCPAPELLG |
| | | | | GPSVFLFPPKPKDTLMISRTPEVTCVVVDVSHE |
| | | | | DPEVKFNWYVDGVEVHNAKTKPCEEQYGSTY |
| | | | | RCVSVLTVLHQDWLNGKEYKCKVSNKALPAPI |
| | | | | EKTISKAKGQPREPQVYTLPPSREEMTKNQVSL |
| | | | | TCLVKGFYPSDIAVEWESNGQPENNYKTTPPV |
| | | | | LDSDGSFFLYSKLTVDKSRWQQGNVFSCSVMH |
| | | | | EALHNHYTQKSLSLSPGKGGGGSGGGGSGGG |
| | | | | GSGGGGSGGGGSGGGGSDKTHTCPPCPAPELL |
| | | | | GGPSVFLFPPKPKDTLMISRTPEVTCVVVDVSH |
| | | | | EDPEVKFNWYVDGVEVHNAKTKPCEEQYGST |
| | | | | YRCVSVLTVLHQDWLNGKEYKCKVSNKALPA |
| | | | | PIEKTISKAKGQPREPQVYTLPPSREEMTKNQV |
| | | | | SLTCLVKGFYPSDIAVEWESNGQPENNYKTTPP |
| | | | | VLDSDGSFFLYSKLTVDKSRWQQGNVFSCSVM |
| | | | | HEALHNHYTQKSLSLSPGK |
| 346. | EPCAM 5-10 x | artificial | Aa | ELVMTQSPSSLTVTAGEKVTMSCKSSQSLLNS |
| | I2C x scFc x I2C x | | | GNQKNYLTWYQQKPGQPPKLLIYWASTRESG |
| | H2 - Full | | | VPDRFTGSGSGTDFTLTISSVQAEDLAVYYCQN |
| | Sequence | | | DYSYPLTFGAGTKLEIKGGGGSGGGGSGGGGS |
| | | | | EVQLLEQSGAELVRPGTSVKISCKASGYAFTNY |
| | | | | WLGWVKQRPGHGLEWIGDIFPGSGNIHYNEKF |
| | | | | KGKATLTADKSSSTAYMQLSSLTFEDSAVYFC |
| | | | | ARLRNWDEPMDYWGQGTTVTVSSGGGGSEV |
| | | | | QLVESGGGLVQPGGSLKLSCAASGFTFNKYAM |
| | | | | NWVRQAPGKGLEWVARIRSKYNNYATYYADS |
| | | | | VKDRFTISRDDSKNTAYLQMNNLKTEDTAVY |
| | | | | YCVRHGNFGNSYISYWAYWGQGTLVTVSSGG |
| | | | | GGSGGGGSGGGGSQTVVTQEPSLTVSPGGTVT |
| | | | | LTCGSSTGAVTSGNYPNWVQQKPGQAPRGLIG |
| | | | | GTKFLAPGTPARFSGSLLGGKAALTLSGVQPED |
| | | | | EAEYYCVLWYSNRWVFGGGTKLTVLGGGGSG |
| | | | | GGGSGGGGSDKTHTCPPCPAPELLGGPSVFLFP |
| | | | | PKPKDTLMISRTPEVTCVVVDVSHEDPEVKFN |
| | | | | WYVDGVEVHNAKTKPCEEQYGSTYRCVSVLT |
| | | | | VLHQDWLNGKEYKCKVSNKALPAPIEKTISKA |
| | | | | KGQPREPQVYTLPPSREEMTKNQVSLTCLVKG |
| | | | | FYPSDIAVEWESNGQPENNYKTTPPVLDSDGSF |
| | | | | FLYSKLTVDKSRWQQGNVFSCSVMHEALHNH |
| | | | | YTQKSLSLSPGKGGGGSGGGGSGGGGSGGGGS |
| | | | | GGGGSGGGGSDKTHTCPPCPAPELLGGPSVFLF |
| | | | | PPKPKDTLMISRTPEVTCVVVDVSHEDPEVKFN |
| | | | | WYVDGVEVHNAKTKPCEEQYGSTYRCVSVLT |
| | | | | VLHQDWLNGKEYKCKVSNKALPAPIEKTISKA |
| | | | | KGQPREPQVYTLPPSREEMTKNQVSLTCLVKG |
| | | | | FYPSDIAVEWESNGQPENNYKTTPPVLDSDGSF |
| | | | | FLYSKLTVDKSRWQQGNVFSCSVMHEALHNH |
| | | | | YTQKSLSLSPGKGGGGSGGGGSGGGGSEVQLV |
| | | | | ESGGGLVQPGGSLKLSCAASGFTFNKYAMNW |
| | | | | VRQAPGKGLEWVARIRSKYNNYATYYADSVK |
| | | | | DRFTISRDDSKNTAYLQMNNLKTEDTAVYYCV |
| | | | | RHGNFGNSYISYWAYWGQGTLVTVSSGGGGS |
| | | | | GGGGSGGGGSQTVVTQEPSLTVSPGGTVTLTC |
| | | | | GSSTGAVTSGNYPNWVQQKPGQAPRGLIGGTK |
| | | | | FLAPGTPARFSGSLLGGKAALTLSGVQPEDEAE |
| | | | | YYCVLWYSNRWVFGGGTKLTVLGGGGSEVQL |
| | | | | LESGGGVVQPGRSLRLSCAASGFTFSSYGMGW |
| | | | | VRQAPGKGLEWVAVISYDGSNKYYADSVKGR |
| | | | | FTISRDNSKNTLYLQMNSLRAEDTAVYYCARE |
| | | | | GAHFGSGSYYPLYYYYAMDVWGQGTTVTVSS |
| | | | | GGGGSGGGGSGGGGSELTLTQSPGTLSLSPGER |
| | | | | ATLSCRASQSVSSSYLAWYQQKPGQAPRLLIY |
| | | | | GASIRATGIPDRFSGSGSGTDFTLTISRLEPEDFA |
| | | | | VYYCQQYGSSPIFTFGPGTKVEIK |
| 347. | EPCAM 5-10 x | artificial | aa | ELVMTQSPSSLTVTAGEKVTMSCKSSQSLLNS |
| | I2Ccc x H2 x | | | GNQKNYLTWYQQKPGQPPKLLIYWASTRESG |
| | I2Ccc - Full | | | VPDRFTGSGSGTDFTLTISSVQAEDLAVYYCQN |
| | Sequence | | | DYSYPLTFGAGTKLEIKGGGGSGGGGSGGGGS |
| | | | | EVQLLEQSGAELVRPGTSVKISCKASGYAFTNY |
| | | | | WLGWVKQRPGHGLEWIGDIFPGSGNIHYNEKF |
| | | | | KGKATLTADKSSSTAYMQLSSLTFEDSAVYFC |
| | | | | ARLRNWDEPMDYWGQGTTVTVSSGGGGSEV |
| | | | | QLVESGGGLVQPGGSLKLSCAASGFTFNKYAM |
| | | | | NWVRQAPGKCLEWVARIRSKYNNYATYYADS |
| | | | | VKDRFTISRDDSKNTAYLQMNNLKTEDTAVY |
| | | | | YCVRHGNFGNSYISYWAYWGQGTLVTVSSGG |
| | | | | GGSGGGGSGGGGSQTVVTQEPSLTVSPGGTVT |
| | | | | LTCGSSTGAVTSGNYPNWVQQKPGQAPRGLIG |
| | | | | GTKFLAPGTPARFSGSLLGGKAALTLSGVQPED |
| | | | | EAEYYCVLWYSNRWVFGCGTKLTVLGGGGSE |
| | | | | VQLLESGGGVVQPGRSLRLSCAASGFTFSSYG |
| | | | | MGWVRQAPGKGLEWVAVISYDGSNKYYADS |
| | | | | VKGRFTISRDNSKNTLYLQMNSLRAEDTAVYY |
| | | | | CAREGAHFGSGSYYPLYYYYAMDVWGQGTT |
| | | | | VTVSSGGGGSGGGGSGGGGSELTLTQSPGTLS |
| | | | | LSPGERATLSCRASQSVSSSYLAWYQQKPGQA |
| | | | | PRLLIYGASIRATGIPDRESGSGSGTDFTLTISRL |
| | | | | EPEDFAVYYCQQYGSSPIFTFGPGTKVEIKSGG |
| | | | | GGSEVQLVESGGGLVQPGGSLKLSCAASGFTF |
| | | | | NKYAMNWVRQAPGKCLEWVARIRSKYNNYA |
| | | | | TYYADSVKDRFTISRDDSKNTAYLQMNNLKTE |
| | | | | DTAVYYCVRHGNFGNSYISYWAYWGQGTLVT |
| | | | | VSSGGGGSGGGGSGGGGSQTVVTQEPSLTVSP |
| | | | | GGTVTLTCGSSTGAVTSGNYPNWVQQKPGQA |
| | | | | PRGLIGGTKFLAPGTPARFSGSLLGGKAALTLS |
| | | | | GVQPEDEAEYYCVLWYSNRWVFGCGTKLTVL |
| 348. | EPCAM 5-10 x | artificial | aa | ELVMTQSPSSLTVTAGEKVTMSCKSSQSLLNS |
| | I2Ccc x scFc x H2 | | | GNQKNYLTWYQQKPGQPPKLLIYWASTRESG |
| | x I2Ccc - Full | | | VPDRFTGSGSGTDFTLTISSVQAEDLAVYYCQN |
| | Sequence | | | DYSYPLTFGAGTKLEIKGGGGSGGGGSGGGGS |
| | | | | EVQLLEQSGAELVRPGTSVKISCKASGYAFTNY |
| | | | | WLGWVKQRPGHGLEWIGDIFPGSGNIHYNEKF |
| | | | | KGKATLTADKSSSTAYMQLSSLTFEDSAVYFC |
| | | | | ARLRNWDEPMDYWGQGTTVTVSSGGGGSEV |
| | | | | QLVESGGGLVQPGGSLKLSCAASGFTFNKYAM |
| | | | | NWVRQAPGKCLEWVARIRSKYNNYATYYADS |
| | | | | VKDRFTISRDDSKNTAYLQMNNLKTEDTAVY |
| | | | | YCVRHGNFGNSYISYWAYWGQGTLVTVSSGG |
| | | | | GGSGGGGSGGGGSQTVVTQEPSLTVSPGGTVT |
| | | | | LTCGSSTGAVTSGNYPNWVQQKPGQAPRGLIG |
| | | | | GTKFLAPGTPARFSGSLLGGKAALTLSGVQPED |
| | | | | EAEYYCVLWYSNRWVFGCGTKLTVLGGGGSG |
| | | | | GGGSGGGGSDKTHTCPPCPAPELLGGPSVFLFP |
| | | | | PKPKDTLMISRTPEVTCVVVDVSHEDPEVKEN |
| | | | | WYVDGVEVHNAKTKPCEEQYGSTYRCVSVLT |
| | | | | VLHQDWLNGKEYKCKVSNKALPAPIEKTISKA |
| | | | | KGQPREPQVYTLPPSREEMTKNQVSLTCLVKG |
| | | | | FYPSDIAVEWESNGQPENNYKTTPPVLDSDGSF |
| | | | | FLYSKLTVDKSRWQQGNVFSCSVMHEALHNH |
| | | | | YTQKSLSLSPGKGGGGSGGGGSGGGGSGGGGS |
| | | | | GGGGSGGGGSDKTHTCPPCPAPELLGGPSVFLF |
| | | | | PPKPKDTLMISRTPEVTCVVVDVSHEDPEVKFN |
| | | | | WYVDGVEVHNAKTKPCEEQYGSTYRCVSVLT |
| | | | | VLHQDWLNGKEYKCKVSNKALPAPIEKTISKA |
| | | | | KGQPREPQVYTLPPSREEMTKNQVSLTCLVKG |
| | | | | FYPSDIAVEWESNGQPENNYKTTPPVLDSDGSF |
| | | | | FLYSKLTVDKSRWQQGNVFSCSVMHEALHNH |
| | | | | YTQKSLSLSPGKGGGGSGGGGSGGGGSEVQLL |
| | | | | ESGGGVVQPGRSLRLSCAASGFTFSSYGMGWV |
| | | | | RQAPGKGLEWVAVISYDGSNKYYADSVKGRF |
| | | | | TISRDNSKNTLYLQMNSLRAEDTAVYYCAREG |
| | | | | AHFGSGSYYPLYYYYAMDVWGQGTTVTVSSG |
| | | | | GGGSGGGGSGGGGSELTLTQSPGTLSLSPGERA |
| | | | | TLSCRASQSVSSSYLAWYQQKPGQAPRLLIYG |
| | | | | ASIRATGIPDRFSGSGSGTDFTLTISRLEPEDFAV |
| | | | | YYCQQYGSSPIFTFGPGTKVEIKSGGGGSEVQL |
| | | | | VESGGGLVQPGGSLKLSCAASGFTFNKYAMN |
| | | | | WVRQAPGKCLEWVARIRSKYNNYATYYADSV |
| | | | | KDRFTISRDDSKNTAYLQMNNLKTEDTAVYYC |
| | | | | VRHGNFGNSYISYWAYWGQGTLVTVSSGGGG |
| | | | | SGGGGSGGGGSQTVVTQEPSLTVSPGGTVTLT |
| | | | | CGSSTGAVTSGNYPNWVQQKPGQAPRGLIGGT |
| | | | | KFLAPGTPARFSGSLLGGKAALTLSGVQPEDEA |
| | | | | EYYCVLWYSNRWVFGCGTKLTVL |
| 349. | EPCAM 5-10 x | artificial | aa | ELVMTQSPSSLTVTAGEKVTMSCKSSQSLLNS |
| | I2Ccc x scFc x | | | GNQKNYLTWYQQKPGQPPKLLIYWASTRESG |
| | I2Ccc x H2 - Full | | | VPDRFTGSGSGTDFTLTISSVQAEDLAVYYCQN |
| | Sequence | | | DYSYPLTFGAGTKLEIKGGGGSGGGGSGGGGS |
| | | | | EVQLLEQSGAELVRPGTSVKISCKASGYAFTNY |
| | | | | WLGWVKQRPGHGLEWIGDIFPGSGNIHYNEKF |
| | | | | KGKATLTADKSSSTAYMQLSSLTFEDSAVYFC |
| | | | | ARLRNWDEPMDYWGQGTTVTVSSGGGGSEV |
| | | | | QLVESGGGLVQPGGSLKLSCAASGFTFNKYAM |
| | | | | NWVRQAPGKCLEWVARIRSKYNNYATYYADS |
| | | | | VKDRFTISRDDSKNTAYLQMNNLKTEDTAVY |
| | | | | YCVRHGNFGNSYISYWAYWGQGTLVTVSSGG |
| | | | | GGSGGGGSGGGGSQTVVTQEPSLTVSPGGTVT |
| | | | | LTCGSSTGAVTSGNYPNWVQQKPGQAPRGLIG |
| | | | | GTKFLAPGTPARFSGSLLGGKAALTLSGVQPED |
| | | | | EAEYYCVLWYSNRWVFGCGTKLTVLGGGGSG |
| | | | | GGGSGGGGSDKTHTCPPCPAPELLGGPSVFLFP |
| | | | | PKPKDTLMISRTPEVTCVVVDVSHEDPEVKEN |
| | | | | WYVDGVEVHNAKTKPCEEQYGSTYRCVSVLT |
| | | | | VLHQDWLNGKEYKCKVSNKALPAPIEKTISKA |
| | | | | KGQPREPQVYTLPPSREEMTKNQVSLTCLVKG |
| | | | | FYPSDIAVEWESNGQPENNYKTTPPVLDSDGSF |
| | | | | FLYSKLTVDKSRWQQGNVFSCSVMHEALHNH |
| | | | | YTQKSLSLSPGKGGGGSGGGGSGGGGSGGGGS |
| | | | | GGGGSGGGGSDKTHTCPPCPAPELLGGPSVFLF |
| | | | | PPKPKDTLMISRTPEVTCVVVDVSHEDPEVKFN |
| | | | | WYVDGVEVHNAKTKPCEEQYGSTYRCVSVLT |
| | | | | VLHQDWLNGKEYKCKVSNKALPAPIEKTISKA |
| | | | | KGQPREPQVYTLPPSREEMTKNQVSLTCLVKG |
| | | | | FYPSDIAVEWESNGQPENNYKTTPPVLDSDGSF |
| | | | | FLYSKLTVDKSRWQQGNVFSCSVMHEALHNH |
| | | | | YTQKSLSLSPGKGGGGSGGGGSGGGGSEVQLV |
| | | | | ESGGGLVQPGGSLKLSCAASGFTFNKYAMNW |
| | | | | VRQAPGKCLEWVARIRSKYNNYATYYADSVK |
| | | | | DRFTISRDDSKNTAYLQMNNLKTEDTAVYYCV |
| | | | | RHGNFGNSYISYWAYWGQGTLVTVSSGGGGS |
| | | | | GGGGSGGGGSQTVVTQEPSLTVSPGGTVTLTC |
| | | | | GSSTGAVTSGNYPNWVQQKPGQAPRGLIGGTK |
| | | | | FLAPGTPARFSGSLLGGKAALTLSGVQPEDEAE |
| | | | | YYCVLWYSNRWVFGCGTKLTVLGGGGSEVQL |
| | | | | LESGGGVVQPGRSLRLSCAASGFTFSSYGMGW |
| | | | | VRQAPGKGLEWVAVISYDGSNKYYADSVKGR |
| | | | | FTISRDNSKNTLYLQMNSLRAEDTAVYYCARE |
| | | | | GAHFGSGSYYPLYYYYAMDVWGQGTTVTVSS |
| | | | | GGGGSGGGGSGGGGSELTLTQSPGTLSLSPGER |
| | | | | ATLSCRASQSVSSSYLAWYQQKPGQAPRLLIY |
| | | | | GASIRATGIPDRFSGSGSGTDFTLTISRLEPEDFA |
| | | | | VYYCQQYGSSPIFTFGPGTKVEIK |
| 350. | EPCAM 5-10 x | artificial | aa | ELVMTQSPSSLTVTAGEKVTMSCKSSQSLLNS |
| | I2Cccx (G4S)10 x | | | GNQKNYLTWYQQKPGQPPKLLIYWASTRESG |
| | H2 x I2Ccc - Full | | | VPDRFTGSGSGTDFTLTISSVQAEDLAVYYCQN |
| | Sequence | | | DYSYPLTFGAGTKLEIKGGGGSGGGGSGGGGS |
| | | | | EVQLLEQSGAELVRPGTSVKISCKASGYAFTNY |
| | | | | WLGWVKQRPGHGLEWIGDIFPGSGNIHYNEKF |
| | | | | KGKATLTADKSSSTAYMQLSSLTFEDSAVYFC |
| | | | | ARLRNWDEPMDYWGQGTTVTVSSGGGGSEV |
| | | | | QLVESGGGLVQPGGSLKLSCAASGFTFNKYAM |
| | | | | NWVRQAPGKCLEWVARIRSKYNNYATYYADS |
| | | | | VKDRFTISRDDSKNTAYLQMNNLKTEDTAVY |
| | | | | YCVRHGNFGNSYISYWAYWGQGTLVTVSSGG |
| | | | | GGSGGGGSGGGGSQTVVTQEPSLTVSPGGTVT |
| | | | | LTCGSSTGAVTSGNYPNWVQQKPGQAPRGLIG |
| | | | | GTKFLAPGTPARFSGSLLGGKAALTLSGVQPED |
| | | | | EAEYYCVLWYSNRWVFGCGTKLTVLGGGGSG |
| | | | | GGGSGGGGSGGGGSGGGGSGGGGSGGGGSGG |
| | | | | GGSGGGGSGGGGSEVQLLESGGGVVQPGRSLR |
| | | | | LSCAASGFTFSSYGMGWVRQAPGKGLEWVAV |
| | | | | ISYDGSNKYYADSVKGRFTISRDNSKNTLYLQ |
| | | | | MNSLRAEDTAVYYCAREGAHFGSGSYYPLYY |
| | | | | YYAMDVWGQGTTVTVSSGGGGSGGGGSGGG |
| | | | | GSELTLTQSPGTLSLSPGERATLSCRASQSVSSS |
| | | | | YLAWYQQKPGQAPRLLIYGASIRATGIPDRESG |
| | | | | SGSGTDFTLTISRLEPEDFAVYYCQQYGSSPIFT |
| | | | | FGPGTKVEIKSGGGGSEVQLVESGGGLVQPGG |
| | | | | SLKLSCAASGFTFNKYAMNWVRQAPGKCLEW |
| | | | | VARIRSKYNNYATYYADSVKDRFTISRDDSKN |
| | | | | TAYLQMNNLKTEDTAVYYCVRHGNFGNSYIS |
| | | | | YWAYWGQGTLVTVSSGGGGSGGGGSGGGGS |
| | | | | QTVVTQEPSLTVSPGGTVTLTCGSSTGAVTSGN |
| | | | | YPNWVQQKPGQAPRGLIGGTKFLAPGTPARFS |
| | | | | GSLLGGKAALTLSGVQPEDEAEYYCVLWYSN |
| | | | | RWVFGCGTKLTVL |
| 351. | EPCAM 5-10 x | artificial | aa | ELVMTQSPSSLTVTAGEKVTMSCKSSQSLLNS |
| | I2Cccx G4Sx | | | GNQKNYLTWYQQKPGQPPKLLIYWASTRESG |
| | PD1xG4S x H2 x | | | VPDRFTGSGSGTDFTLTISSVQAEDLAVYYCQN |
| | I2Ccc - Full | | | DYSYPLTFGAGTKLEIKGGGGSGGGGSGGGGS |
| | Sequence | | | EVQLLEQSGAELVRPGTSVKISCKASGYAFTNY |
| | | | | WLGWVKQRPGHGLEWIGDIFPGSGNIHYNEKF |
| | | | | KGKATLTADKSSSTAYMQLSSLTFEDSAVYFC |
| | | | | ARLRNWDEPMDYWGQGTTVTVSSGGGGSEV |
| | | | | QLVESGGGLVQPGGSLKLSCAASGFTFNKYAM |
| | | | | NWVRQAPGKCLEWVARIRSKYNNYATYYADS |
| | | | | VKDRFTISRDDSKNTAYLQMNNLKTEDTAVY |
| | | | | YCVRHGNFGNSYISYWAYWGQGTLVTVSSGG |
| | | | | GGSGGGGSGGGGSQTVVTQEPSLTVSPGGTVT |
| | | | | LTCGSSTGAVTSGNYPNWVQQKPGQAPRGLIG |
| | | | | GTKFLAPGTPARFSGSLLGGKAALTLSGVQPED |
| | | | | EAEYYCVLWYSNRWVFGCGTKLTVLGGGGSL |
| | | | | DSPDRPWNPPTFSPALLVVTEGDNATFTCSFSN |
| | | | | TSESFVLNWYRMSPSNQTDKLAAFPEDRSQPG |
| | | | | QDCRFRVTQLPNGRDFHMSVVRARRNDSGTY |
| | | | | LCGAISLAPKAQIKESLRAELRVTERRAEVPTA |
| | | | | HPSPSPRPAGQFQGGGGSEVQLLESGGGVVQP |
| | | | | GRSLRLSCAASGFTFSSYGMGWVRQAPGKGLE |
| | | | | WVAVISYDGSNKYYADSVKGRFTISRDNSKNT |
| | | | | LYLQMNSLRAEDTAVYYCAREGAHFGSGSYY |
| | | | | PLYYYYAMDVWGQGTTVTVSSGGGGSGGGG |
| | | | | SGGGGSELTLTQSPGTLSLSPGERATLSCRASQ |
| | | | | SVSSSYLAWYQQKPGQAPRLLIYGASIRATGIP |
| | | | | DRFSGSGSGTDFTLTISRLEPEDFAVYYCQQYG |
| | | | | SSPIFTFGPGTKVEIKSGGGGSEVQLVESGGGL |
| | | | | VQPGGSLKLSCAASGFTFNKYAMNWVRQAPG |
| | | | | KCLEWVARIRSKYNNYATYYADSVKDRFTISR |
| | | | | DDSKNTAYLQMNNLKTEDTAVYYCVRHGNFG |
| | | | | NSYISYWAYWGQGTLVTVSSGGGGSGGGGSG |
| | | | | GGGSQTVVTQEPSLTVSPGGTVTLTCGSSTGA |
| | | | | VTSGNYPNWVQQKPGQAPRGLIGGTKFLAPGT |
| | | | | PARFSGSLLGGKAALTLSGVQPEDEAEYYCVL |
| | | | | WYSNRWVFGCGTKLTVL |
| 352. | EpCAM_5- | artificial | aa | ELVMTQSPSSLTVTAGEKVTMSCKSSQSLLNS |
| | 10_x(EAAAK)10 | | | GNQKNYLTWYQQKPGQPPKLLIYWASTRESG |
| | _x | | | VPDRFTGSGSGTDFTLTISSVQAEDLAVYYCQN |
| | I2Ccc_xG4_xscFc | | | DYSYPLTFGAGTKLEIKGGGGSGGGGSGGGGS |
| | _xG4_xMSLN_H | | | EVQLLEQSGAELVRPGTSVKISCKASGYAFTNY |
| | _x(EAAAK)10_x | | | WLGWVKQRPGHGLEWIGDIFPGSGNIHYNEKF |
| | I2Ccc - Full | | | KGKATLTADKSSSTAYMQLSSLTFEDSAVYFC |
| | Sequence | | | ARLRNWDEPMDYWGQGTTVTVSSGEAAAKE |
| | | | | AAAKEAAAKEAAAKEAAAKEAAAKEAAAKE |
| | | | | AAAKEAAAKEAAAKEVQLVESGGGLVQPGGS |
| | | | | LKLSCAASGFTFNKYAMNWVRQAPGKCLEWV |
| | | | | ARIRSKYNNYATYYADSVKDRFTISRDDSKNT |
| | | | | AYLQMNNLKTEDTAVYYCVRHGNFGNSYISY |
| | | | | WAYWGQGTLVTVSSGGGGSGGGGSGGGGSQ |
| | | | | TVVTQEPSLTVSPGGTVTLTCGSSTGAVTSGNY |
| | | | | PNWVQQKPGQAPRGLIGGTKFLAPGTPARFSG |
| | | | | SLLGGKAALTLSGVQPEDEAEYYCVLWYSNR |
| | | | | WVFGCGTKLTVLGGGGDKTHTCPPCPAPELLG |
| | | | | GPSVFLFPPKPKDTLMISRTPEVTCVVVDVSHE |
| | | | | DPEVKFNWYVDGVEVHNAKTKPCEEQYGSTY |
| | | | | RCVSVLTVLHQDWLNGKEYKCKVSNKALPAPI |
| | | | | EKTISKAKGQPREPQVYTLPPSREEMTKNQVSL |
| | | | | TCLVKGFYPSDIAVEWESNGQPENNYKTTPPV |
| | | | | LDSDGSFFLYSKLTVDKSRWQQGNVFSCSVMH |
| | | | | EALHNHYTQKSLSLSPGKGGGGSGGGGSGGG |
| | | | | GSGGGGSGGGGSGGGGSDKTHTCPPCPAPELL |
| | | | | GGPSVFLFPPKPKDTLMISRTPEVTCVVVDVSH |
| | | | | EDPEVKFNWYVDGVEVHNAKTKPCEEQYGST |
| | | | | YRCVSVLTVLHQDWLNGKEYKCKVSNKALPA |
| | | | | PIEKTISKAKGQPREPQVYTLPPSREEMTKNQV |
| | | | | SLTCLVKGFYPSDIAVEWESNGQPENNYKTTPP |
| | | | | VLDSDGSFFLYSKLTVDKSRWQQGNVFSCSVM |
| | | | | HEALHNHYTQKSLSLSPGKGGGGEVQLLESGG |
| | | | | GVVQPGRSLRLSCAASGFTFSSYGMGWVRQAP |
| | | | | GKGLEWVAVISYDGSNKYYADSVKGRFTISRD |
| | | | | NSKNTLYLQMNSLRAEDTAVYYCAREGAHFG |
| | | | | SGSYYPLYYYYAMDVWGQGTTVTVSSGGGGS |
| | | | | GGGGSGGGGSELTLTQSPGTLSLSPGERATLSC |
| | | | | RASQSVSSSYLAWYQQKPGQAPRLLIYGASIRA |
| | | | | TGIPDRFSGSGSGTDFTLTISRLEPEDFAVYYCQ |
| | | | | QYGSSPIFTFGPGTKVEIKSGEAAAKEAAAKEA |
| | | | | AAKEAAAKEAAAKEAAAKEAAAKEAAAKEA |
| | | | | AAKEAAAKEVQLVESGGGLVQPGGSLKLSCA |
| | | | | ASGFTFNKYAMNWVRQAPGKCLEWVARIRSK |
| | | | | YNNYATYYADSVKDRFTISRDDSKNTAYLQM |
| | | | | NNLKTEDTAVYYCVRHGNFGNSYISYWAYWG |
| | | | | QGTLVTVSSGGGGSGGGGSGGGGSQTVVTQEP |
| | | | | SLTVSPGGTVTLTCGSSTGAVTSGNYPNWVQQ |
| | | | | KPGQAPRGLIGGTKFLAPGTPARFSGSLLGGKA |
| | | | | ALTLSGVQPEDEAEYYCVLWYSNRWVFGCGT |
| | | | | KLTVL |
| 353. | EpCAM_5- | artificial | aa | ELVMTQSPSSLTVTAGEKVTMSCKSSQSLLNS |
| | 10_x(G4S)3_x | | | GNQKNYLTWYQQKPGQPPKLLIYWASTRESG |
| | I2Ccc_xG4_xscFc | | | VPDRFTGSGSGTDFTLTISSVQAEDLAVYYCQN |
| | _xG4_xMSLN_H | | | DYSYPLTFGAGTKLEIKGGGGSGGGGSGGGGS |
| | _x(G4S)3_x | | | EVQLLEQSGAELVRPGTSVKISCKASGYAFTNY |
| | I2Ccc- Full | | | WLGWVKQRPGHGLEWIGDIFPGSGNIHYNEKF |
| | Sequence | | | KGKATLTADKSSSTAYMQLSSLTFEDSAVYFC |
| | | | | ARLRNWDEPMDYWGQGTTVTVSSGGGGSGG |
| | | | | GGSGGGGSEVQLVESGGGLVQPGGSLKLSCAA |
| | | | | SGFTFNKYAMNWVRQAPGKCLEWVARIRSKY |
| | | | | NNYATYYADSVKDRFTISRDDSKNTAYLQMN |
| | | | | NLKTEDTAVYYCVRHGNFGNSYISYWAYWGQ |
| | | | | GTLVTVSSGGGGSGGGGSGGGGSQTVVTQEPS |
| | | | | LTVSPGGTVTLTCGSSTGAVTSGNYPNWVQQK |
| | | | | PGQAPRGLIGGTKFLAPGTPARFSGSLLGGKAA |
| | | | | LTLSGVQPEDEAEYYCVLWYSNRWVFGCGTK |
| | | | | LTVLGGGGDKTHTCPPCPAPELLGGPSVFLFPP |
| | | | | KPKDTLMISRTPEVTCVVVDVSHEDPEVKFNW |
| | | | | YVDGVEVHNAKTKPCEEQYGSTYRCVSVLTV |
| | | | | LHQDWLNGKEYKCKVSNKALPAPIEKTISKAK |
| | | | | GQPREPQVYTLPPSREEMTKNQVSLTCLVKGF |
| | | | | YPSDIAVEWESNGQPENNYKTTPPVLDSDGSFF |
| | | | | LYSKLTVDKSRWQQGNVFSCSVMHEALHNHY |
| | | | | TQKSLSLSPGKGGGGSGGGGSGGGGSGGGGSG |
| | | | | GGGSGGGGSDKTHTCPPCPAPELLGGPSVFLFP |
| | | | | PKPKDTLMISRTPEVTCVVVDVSHEDPEVKEN |
| | | | | WYVDGVEVHNAKTKPCEEQYGSTYRCVSVLT |
| | | | | VLHQDWLNGKEYKCKVSNKALPAPIEKTISKA |
| | | | | KGQPREPQVYTLPPSREEMTKNQVSLTCLVKG |
| | | | | FYPSDIAVEWESNGQPENNYKTTPPVLDSDGSF |
| | | | | FLYSKLTVDKSRWQQGNVFSCSVMHEALHNH |
| | | | | YTQKSLSLSPGKGGGGEVQLLESGGGVVQPGR |
| | | | | SLRLSCAASGFTFSSYGMGWVRQAPGKGLEW |
| | | | | VAVISYDGSNKYYADSVKGRFTISRDNSKNTL |
| | | | | YLQMNSLRAEDTAVYYCAREGAHFGSGSYYP |
| | | | | LYYYYAMDVWGQGTTVTVSSGGGGSGGGGS |
| | | | | GGGGSELTLTQSPGTLSLSPGERATLSCRASQS |
| | | | | VSSSYLAWYQQKPGQAPRLLIYGASIRATGIPD |
| | | | | RFSGSGSGTDFTLTISRLEPEDFAVYYCQQYGS |
| | | | | SPIFTFGPGTKVEIKSGGGGSGGGGSGGGGSEV |
| | | | | QLVESGGGLVQPGGSLKLSCAASGFTFNKYAM |
| | | | | NWVRQAPGKCLEWVARIRSKYNNYATYYADS |
| | | | | VKDRFTISRDDSKNTAYLQMNNLKTEDTAVY |
| | | | | YCVRHGNFGNSYISYWAYWGQGTLVTVSSGG |
| | | | | GGSGGGGSGGGGSQTVVTQEPSLTVSPGGTVT |
| | | | | LTCGSSTGAVTSGNYPNWVQQKPGQAPRGLIG |
| | | | | GTKFLAPGTPARFSGSLLGGKAALTLSGVQPED |
| | | | | EAEYYCVLWYSNRWVFGCGTKLTVL |
| 354. | EpCAM_5-10_x | artificial | aa | ELVMTQSPSSLTVTAGEKVTMSCKSSQSLLNS |
| | I2Ccc_xscFc_xM | | | GNQKNYLTWYQQKPGQPPKLLIYWASTRESG |
| | SLN_H2_x I2Ccc - | | | VPDRFTGSGSGTDFTLTISSVQAEDLAVYYCQN |
| | Full Sequence | | | DYSYPLTFGAGTKLEIKGGGGSGGGGSGGGGS |
| | | | | EVQLLEQSGAELVRPGTSVKISCKASGYAFTNY |
| | | | | WLGWVKQRPGHGLEWIGDIFPGSGNIHYNEKF |
| | | | | KGKATLTADKSSSTAYMQLSSLTFEDSAVYFC |
| | | | | ARLRNWDEPMDYWGQGTTVTVSSGGGGSEV |
| | | | | QLVESGGGLVQPGGSLKLSCAASGFTFNKYAM |
| | | | | NWVRQAPGKCLEWVARIRSKYNNYATYYADS |
| | | | | VKDRFTISRDDSKNTAYLQMNNLKTEDTAVY |
| | | | | YCVRHGNFGNSYISYWAYWGQGTLVTVSSGG |
| | | | | GGSGGGGSGGGGSQTVVTQEPSLTVSPGGTVT |
| | | | | LTCGSSTGAVTSGNYPNWVQQKPGQAPRGLIG |
| | | | | GTKFLAPGTPARFSGSLLGGKAALTLSGVQPED |
| | | | | EAEYYCVLWYSNRWVFGCGTKLTVLDKTHTC |
| | | | | PPCPAPELLGGPSVFLFPPKPKDTLMISRTPEVT |
| | | | | CVVVDVSHEDPEVKFNWYVDGVEVHNAKTKP |
| | | | | CEEQYGSTYRCVSVLTVLHQDWLNGKEYKCK |
| | | | | VSNKALPAPIEKTISKAKGQPREPQVYTLPPSRE |
| | | | | EMTKNQVSLTCLVKGFYPSDIAVEWESNGQPE |
| | | | | NNYKTTPPVLDSDGSFFLYSKLTVDKSRWQQG |
| | | | | NVFSCSVMHEALHNHYTQKSLSLSPGKGGGGS |
| | | | | GGGGSGGGGSGGGGSGGGGSGGGGSDKTHTC |
| | | | | PPCPAPELLGGPSVFLFPPKPKDTLMISRTPEVT |
| | | | | CVVVDVSHEDPEVKFNWYVDGVEVHNAKTKP |
| | | | | CEEQYGSTYRCVSVLTVLHQDWLNGKEYKCK |
| | | | | VSNKALPAPIEKTISKAKGQPREPQVYTLPPSRE |
| | | | | EMTKNQVSLTCLVKGFYPSDIAVEWESNGQPE |
| | | | | NNYKTTPPVLDSDGSFFLYSKLTVDKSRWQQG |
| | | | | NVFSCSVMHEALHNHYTQKSLSLSPGKEVQLL |
| | | | | ESGGGVVQPGRSLRLSCAASGFTFSSYGMGWV |
| | | | | RQAPGKGLEWVAVISYDGSNKYYADSVKGRF |
| | | | | TISRDNSKNTLYLQMNSLRAEDTAVYYCAREG |
| | | | | AHFGSGSYYPLYYYYAMDVWGQGTTVTVSSG |
| | | | | GGGSGGGGSGGGGSELTLTQSPGTLSLSPGERA |
| | | | | TLSCRASQSVSSSYLAWYQQKPGQAPRLLIYG |
| | | | | ASIRATGIPDRFSGSGSGTDFTLTISRLEPEDFAV |
| | | | | YYCQQYGSSPIFTFGPGTKVEIKSGGGGSEVQL |
| | | | | VESGGGLVQPGGSLKLSCAASGFTFNKYAMN |
| | | | | WVRQAPGKCLEWVARIRSKYNNYATYYADSV |
| | | | | KDRFTISRDDSKNTAYLQMNNLKTEDTAVYYC |
| | | | | VRHGNFGNSYISYWAYWGQGTLVTVSSGGGG |
| | | | | SGGGGSGGGGSQTVVTQEPSLTVSPGGTVTLT |
| | | | | CGSSTGAVTSGNYPNWVQQKPGQAPRGLIGGT |
| | | | | KFLAPGTPARFSGSLLGGKAALTLSGVQPEDEA |
| | | | | EYYCVLWYSNRWVFGCGTKLTVL |
| 355. | EpCAM_x(G4S)1 | artificial | Aa | ELVMTQSPSSLTVTAGEKVTMSCKSSQSLLNS |
| | 0_x | | | GNQKNYLTWYQQKPGQPPKLLIYWASTRESG |
| | I2Ccc_xscFc_x | | | VPDRFTGSGSGTDFTLTISSVQAEDLAVYYCQN |
| | I2Ccc_x(G4S)10_ | | | DYSYPLTFGAGTKLEIKGGGGSGGGGSGGGGS |
| | x_MSLN_H2 - | | | EVQLLEQSGAELVRPGTSVKISCKASGYAFTNY |
| | Full Sequence | | | WLGWVKQRPGHGLEWIGDIFPGSGNIHYNEKF |
| | | | | KGKATLTADKSSSTAYMQLSSLTFEDSAVYFC |
| | | | | ARLRNWDEPMDYWGQGTTVTVSSGGGGSGG |
| | | | | GGSGGGGSGGGGSGGGGSGGGGSGGGGSGGG |
| | | | | GSGGGGSGGGGSEVQLVESGGGLVQPGGSLKL |
| | | | | SCAASGFTFNKYAMNWVRQAPGKCLEWVARI |
| | | | | RSKYNNYATYYADSVKDRFTISRDDSKNTAYL |
| | | | | QMNNLKTEDTAVYYCVRHGNFGNSYISYWAY |
| | | | | WGQGTLVTVSSGGGGSGGGGSGGGGSQTVVT |
| | | | | QEPSLTVSPGGTVTLTCGSSTGAVTSGNYPNW |
| | | | | VQQKPGQAPRGLIGGTKFLAPGTPARFSGSLLG |
| | | | | GKAALTLSGVQPEDEAEYYCVLWYSNRWVFG |
| | | | | CGTKLTVLGGGGDKTHTCPPCPAPELLGGPSV |
| | | | | FLFPPKPKDTLMISRTPEVTCVVVDVSHEDPEV |
| | | | | KFNWYVDGVEVHNAKTKPCEEQYGSTYRCVS |
| | | | | VLTVLHQDWLNGKEYKCKVSNKALPAPIEKTI |
| | | | | SKAKGQPREPQVYTLPPSREEMTKNQVSLTCL |
| | | | | VKGFYPSDIAVEWESNGQPENNYKTTPPVLDS |
| | | | | DGSFFLYSKLTVDKSRWQQGNVFSCSVMHEA |
| | | | | LHNHYTQKSLSLSPGKGGGGSGGGGSGGGGS |
| | | | | GGGGSGGGGSGGGGSDKTHTCPPCPAPELLGG |
| | | | | PSVFLFPPKPKDTLMISRTPEVTCVVVDVSHED |
| | | | | PEVKFNWYVDGVEVHNAKTKPCEEQYGSTYR |
| | | | | CVSVLTVLHQDWLNGKEYKCKVSNKALPAPIE |
| | | | | KTISKAKGQPREPQVYTLPPSREEMTKNQVSLT |
| | | | | CLVKGFYPSDIAVEWESNGQPENNYKTTPPVL |
| | | | | DSDGSFFLYSKLTVDKSRWQQGNVFSCSVMHE |
| | | | | ALHNHYTQKSLSLSPGKGGGGEVQLVESGGGL |
| | | | | VQPGGSLKLSCAASGFTFNKYAMNWVRQAPG |
| | | | | KCLEWVARIRSKYNNYATYYADSVKDRFTISR |
| | | | | DDSKNTAYLQMNNLKTEDTAVYYCVRHGNFG |
| | | | | NSYISYWAYWGQGTLVTVSSGGGGSGGGGSG |
| | | | | GGGSQTVVTQEPSLTVSPGGTVTLTCGSSTGA |
| | | | | VTSGNYPNWVQQKPGQAPRGLIGGTKFLAPGT |
| | | | | PARFSGSLLGGKAALTLSGVQPEDEAEYYCVL |
| | | | | WYSNRWVFGCGTKLTVLSGGGGSGGGGSGGG |
| | | | | GSGGGGSGGGGSGGGGSGGGGSGGGGSGGGG |
| | | | | SEVQLLESGGGVVQPGRSLRLSCAASGFTFSSY |
| | | | | GMGWVRQAPGKGLEWVAVISYDGSNKYYAD |
| | | | | SVKGRFTISRDNSKNTLYLQMNSLRAEDTAVY |
| | | | | YCAREGAHFGSGSYYPLYYYYAMDVWGQGT |
| | | | | TVTVSSGGGGSGGGGSGGGGSELTLTQSPGTL |
| | | | | SLSPGERATLSCRASQSVSSSYLAWYQQKPGQ |
| | | | | APRLLIYGASIRATGIPDRFSGSGSGTDFTLTISR |
| | | | | LEPEDFAVYYCQQYGSSPIFTFGPGTKVEIK |
| 356. | CD20_99-E5_CC - | artificial | Aa | SYWMH |
| | HCDR1 | | | |
| 357. | CD20_99-E5_CC - | artificial | Aa | YITPSTGYTEYNQKFKG |
| | HCDR2 | | | |
| 358. | CD20_99-E5_CC - | artificial | Aa | VHDYDRAMEY |
| | HCDR3 | | | |
| 359. | CD20_99-E5_CC - | artificial | Aa | KASQDINKYIA |
| | LCDR1 | | | |
| 360. | CD20_99-E5_CC - | artificial | Aa | YTSTLQP |
| | LCDR2 | | | |
| 361. | CD20_99-E5_CC - | artificial | Aa | LQYASYPFT |
| | LCDR3 | | | |
| 362. | CD20_99-E5_CC - | artificial | Aa | QVQLVQSGAEVKKPGASVKVSCKASGYTFTSY |
| | VH | | | WMHWVRQAPGQCLEWIGYITPSTGYTEYNQK |
| | | | | FKGRVTMTRDKSTSTVYMELSSLTSEDTAVYY |
| | | | | CARVHDYDRAMEYWGQGTTVTVSS |
| 363. | CD20_99-E5_CC - | artificial | Aa | DIQMTQSPSSLSASVGDRVTITCKASQDINKYIA |
| | VL | | | WYQQKPGKGPKLLIYYTSTLQPGVPSRFSGSGS |
| | | | | GTDFTFTISSLQPEDIATYYCLQYASYPFTFGCG |
| | | | | TRLEIK |
| 364. | CD20_99-E5_CC | artificial | aa | EIQMTQSPSSLSASVGDRVTITCKASQDINKYIA |
| | EI - VL | | | WYQQKPGKGPKLLIYYTSTLQPGVPSRFSGSGS |
| | | | | GTDFTFTISSLQPEDIATYYCLQYASYPFTFGCG |
| | | | | TRLEIK |
| 365. | CD22_28- | artificial | aa | SYGIS |
| | B7N655S_CC - | | | |
| | HCDR1 | | | |
| 366. | CD22_28- | artificial | aa | WISAYSGNAIYAQKLQG |
| | B7N655S_CC - | | | |
| | HCDR2 | | | |
| 367. | CD22_28- | artificial | aa | DPDYYGSGSYSDY |
| | B7N655S_CC - | | | |
| | HCDR3 | | | |
| 368. | CD22_28- | artificial | aa | RASQSVSSNLA |
| | B7N655S_CC - | | | |
| | LCDR1 | | | |
| 369. | CD22_28- | artificial | aa | GASSRAT |
| | B7N655S_CC - | | | |
| | LCDR2 | | | |
| 370. | CD22_28- | artificial | aa | QQYHSWPLLT |
| | B7N655S_CC - | | | |
| | LCDR3 | | | |
| 371. | CD22_28- | artificial | aa | QVQLVQSGAEVKKPGASVKVSCKASGYTFTSY |
| | B7N655S_CC - | | | GISWVRQAPGQCLEWMGWISAYSGNAIYAQK |
| | VH | | | LQGRVTMTRDTSTSTAYMELRSLRSDDTAVYY |
| | | | | CARDPDYYGSGSYSDYWGQGTLVTVSS |
| 372. | CD22_28- | artificial | Aa | EIVLTQSPATLSVSPGERATLSCRASQSVSSNLA |
| | B7N655S_CC - | | | WYQQKPGQAPRLLIYGASSRATGIPARFSGSGS |
| | VL | | | GTEFTLTISSLQSEDFAVYYCQQYHSWPLLTFG |
| | | | | CGTKVEIK |
| 373. | CD22_28- | artificial | Aa | QVQLVQSGAEVKKPGASVKVSCKASGYTFTSY |
| | B7N655SCC_x_I | | | GISWVRQAPGQCLEWMGWISAYSGNAIYAQK |
| | 2C_x_(G4S)3_x_s | | | LQGRVTMTRDTSTSTAYMELRSLRSDDTAVYY |
| | cFc_x_(G4S)3_x_ | | | CARDPDYYGSGSYSDYWGQGTLVTVSSGGGG |
| | CD20_99- | | | SGGGGSGGGGSEIVLTQSPATLSVSPGERATLS |
| | E5_CC_x_I2C - | | | CRASQSVSSNLAWYQQKPGQAPRLLIYGASSR |
| | Full Sequence | | | ATGIPARFSGSGSGTEFTLTISSLQSEDFAVYYC |
| | | | | QQYHSWPLLTFGCGTKVEIKSGGGGSEVQLVE |
| | | | | SGGGLVQPGGSLKLSCAASGFTFNKYAMNWV |
| | | | | RQAPGKGLEWVARIRSKYNNYATYYADSVKD |
| | | | | RFTISRDDSKNTAYLQMNNLKTEDTAVYYCVR |
| | | | | HGNFGNSYISYWAYWGQGTLVTVSSGGGGSG |
| | | | | GGGSGGGGSQTVVTQEPSLTVSPGGTVTLTCG |
| | | | | SSTGAVTSGNYPNWVQQKPGQAPRGLIGGTKF |
| | | | | LAPGTPARFSGSLLGGKAALTLSGVQPEDEAE |
| | | | | YYCVLWYSNRWVFGGGTKLTVLGGGGSGGG |
| | | | | GSGGGGSDKTHTCPPCPAPELLGGPSVFLFPPK |
| | | | | PKDTLMISRTPEVTCVVVDVSHEDPEVKFNWY |
| | | | | VDGVEVHNAKTKPCEEQYGSTYRCVSVLTVL |
| | | | | HQDWLNGKEYKCKVSNKALPAPIEKTISKAKG |
| | | | | QPREPQVYTLPPSREEMTKNQVSLTCLVKGFY |
| | | | | PSDIAVEWESNGQPENNYKTTPPVLDSDGSFFL |
| | | | | YSKLTVDKSRWQQGNVFSCSVMHEALHNHYT |
| | | | | QKSLSLSPGKGGGGSGGGGSGGGGSGGGGSG |
| | | | | GGGSGGGGSDKTHTCPPCPAPELLGGPSVFLFP |
| | | | | PKPKDTLMISRTPEVTCVVVDVSHEDPEVKEN |
| | | | | WYVDGVEVHNAKTKPCEEQYGSTYRCVSVLT |
| | | | | VLHQDWLNGKEYKCKVSNKALPAPIEKTISKA |
| | | | | KGQPREPQVYTLPPSREEMTKNQVSLTCLVKG |
| | | | | FYPSDIAVEWESNGQPENNYKTTPPVLDSDGSF |
| | | | | FLYSKLTVDKSRWQQGNVFSCSVMHEALHNH |
| | | | | YTQKSLSLSPGKGGGGSGGGGSGGGGSQVQL |
| | | | | VQSGAEVKKPGASVKVSCKASGYTFTSYWMH |
| | | | | WVRQAPGQCLEWIGYITPSTGYTEYNQKFKGR |
| | | | | VTMTRDKSTSTVYMELSSLTSEDTAVYYCARV |
| | | | | HDYDRAMEYWGQGTTVTVSSGGGGSGGGGS |
| | | | | GGGGSDIQMTQSPSSLSASVGDRVTITCKASQD |
| | | | | INKYIAWYQQKPGKGPKLLIYYTSTLQPGVPSR |
| | | | | FSGSGSGTDFTFTISSLQPEDIATYYCLQYASYP |
| | | | | FTFGCGTRLEIKSGGGGSEVQLVESGGGLVQPG |
| | | | | GSLKLSCAASGFTFNKYAMNWVRQAPGKGLE |
| | | | | WVARIRSKYNNYATYYADSVKDRFTISRDDSK |
| | | | | NTAYLQMNNLKTEDTAVYYCVRHGNFGNSYI |
| | | | | SYWAYWGQGTLVTVSSGGGGSGGGGSGGGG |
| | | | | SQTVVTQEPSLTVSPGGTVTLTCGSSTGAVTSG |
| | | | | NYPNWVQQKPGQAPRGLIGGTKFLAPGTPARF |
| | | | | SGSLLGGKAALTLSGVQPEDEAEYYCVLWYSN |
| | | | | RWVFGGGTKLTVL |
| 374. | CD22_28- | artificial | Aa | QVQLVQSGAEVKKPGASVKVSCKASGYTFTSY |
| | B7_N655S_CC_x | | | GISWVRQAPGQCLEWMGWISAYSGNAIYAQK |
| | I2E_x_(G4Q)3_x | | | LQGRVTMTRDTSTSTAYMELRSLRSDDTAVYY |
| | scFc_x_(G4Q)3x | | | CARDPDYYGSGSYSDYWGQGTLVTVSSGGGG |
| | CD20_99- | | | QGGGGQGGGGQEIVLTQSPATLSVSPGERATL |
| | E5_CC_x_I2E_EI | | | SCRASQSVSSNLAWYQQKPGQAPRLLIYGASS |
| | mod - Full | | | RATGIPARFSGSGSGTEFTLTISSLQSEDFAVYY |
| | Sequence | | | CQQYHSWPLLTFGCGTKVEIKSGGGGQEVQLV |
| | | | | ESGGGLVQPGGSLKLSCAASGFTFNKYAINWV |
| | | | | RQAPGKGLEWVARIRSKYNNYATYYADAVKD |
| | | | | RFTISRDDSKNTVYLQMNNLKTEDTAVYYCAR |
| | | | | AGNFGSSYISYWAYWGQGTLVTVSSGGGGQG |
| | | | | GGGQGGGGQQTVVTQEPSLTVSPGGTVTITCG |
| | | | | SSTGAVTSGNYPNWVQKKPGQAPRGLIGGTKF |
| | | | | LAPGTPARFSGSLSGGKAALTLSGVQPEDEAEY |
| | | | | YCVLWYSNRWVFGSGTKLTVLGGGGQGGGG |
| | | | | QGGGGQCPPCPAPELLGGPSVFLFPPKPKDTLM |
| | | | | ISRTPEVTCVVVDVSHEEPEVKFNWYVDGVEV |
| | | | | HNAKTKPCEEQYGSTYRCVSVLTVLHQDWLN |
| | | | | GKEYKCKVSNKALPAPIEKTISKAKGQPREPQV |
| | | | | YTLPPSREEMTKNQVSLTCLVKGFYPSDIAVE |
| | | | | WESNGQPENNYKTTPPVLDSDGSFFLYSKLTV |
| | | | | DKSRWQQGNVFSCSVMHEALHNHYTQKSLSL |
| | | | | SPGKGGGGQGGGGQGGGGQGGGGQGGGGQG |
| | | | | GGGQCPPCPAPELLGGPSVFLFPPKPKDTLMIS |
| | | | | RTPEVTCVVVDVSHEEPEVKFNWYVDGVEVH |
| | | | | NAKTKPCEEQYGSTYRCVSVLTVLHQDWLNG |
| | | | | KEYKCKVSNKALPAPIEKTISKAKGQPREPQVY |
| | | | | TLPPSREEMTKNQVSLTCLVKGFYPSDIAVEWE |
| | | | | SNGQPENNYKTTPPVLDSDGSFFLYSKLTVDKS |
| | | | | RWQQGNVFSCSVMHEALHNHYTQKSLSLSPG |
| | | | | KGGGGQGGGGQGGGGQQVQLVQSGAEVKKP |
| | | | | GASVKVSCKASGYTFTSYWMHWVRQAPGQC |
| | | | | LEWIGYITPSTGYTEYNQKFKGRVTMTRDKST |
| | | | | STVYMELSSLTSEDTAVYYCARVHDYDRAME |
| | | | | YWGQGTTVTVSSGGGGQGGGGQGGGGQEIQ |
| | | | | MTQSPSSLSASVGDRVTITCKASQDINKYIAWY |
| | | | | QQKPGKGPKLLIYYTSTLQPGVPSRFSGSGSGT |
| | | | | DFTFTISSLQPEDIATYYCLQYASYPFTFGCGTR |
| | | | | LEIKSGGGGQEVQLVESGGGLVQPGGSLKLSC |
| | | | | AASGFTFNKYAINWVRQAPGKGLEWVARIRSK |
| | | | | YNNYATYYADAVKDRFTISRDDSKNTVYLQM |
| | | | | NNLKTEDTAVYYCARAGNFGSSYISYWAYWG |
| | | | | QGTLVTVSSGGGGQGGGGQGGGGQQTVVTQE |
| | | | | PSLTVSPGGTVTITCGSSTGAVTSGNYPNWVQ |
| | | | | KKPGQAPRGLIGGTKFLAPGTPARFSGSLSGGK |
| | | | | AALTLSGVQPEDEAEYYCVLWYSNRWVFGSG |
| | | | | TKLTVL |
| 375. | CD22_28- | artificial | Aa | QVQLVQSGAEVKKPGASVKVSCKASGYTFTSY |
| | B7_N655S_CC_x | | | GISWVRQAPGQCLEWMGWISAYSGNAIYAQK |
| | I2E_x_G4_x_sc | | | LQGRVTMTRDTSTSTAYMELRSLRSDDTAVYY |
| | Fc_x_G4_x_CD2 | | | CARDPDYYGSGSYSDYWGQGTLVTVSSGGGG |
| | 0_99- | | | QGGGGQGGGGQEIVLTQSPATLSVSPGERATL |
| | E5_CC_x_I2E_G | | | SCRASQSVSSNLAWYQQKPGQAPRLLIYGASS |
| | Q_EImod - Full | | | RATGIPARFSGSGSGTEFTLTISSLQSEDFAVYY |
| | Sequence | | | CQQYHSWPLLTFGCGTKVEIKSGGGGQEVQLV |
| | | | | ESGGGLVQPGGSLKLSCAASGFTFNKYAINWV |
| | | | | RQAPGKGLEWVARIRSKYNNYATYYADAVKD |
| | | | | RFTISRDDSKNTVYLQMNNLKTEDTAVYYCAR |
| | | | | AGNFGSSYISYWAYWGQGTLVTVSSGGGGQG |
| | | | | GGGQGGGGQQTVVTQEPSLTVSPGGTVTITCG |
| | | | | SSTGAVTSGNYPNWVQKKPGQAPRGLIGGTKF |
| | | | | LAPGTPARFSGSLSGGKAALTLSGVQPEDEAEY |
| | | | | YCVLWYSNRWVFGSGTKLTVLGGGGCPPCPA |
| | | | | PELLGGPSVFLFPPKPKDTLMISRTPEVTCVVV |
| | | | | DVSHEEPEVKFNWYVDGVEVHNAKTKPCEEQ |
| | | | | YGSTYRCVSVLTVLHQDWLNGKEYKCKVSNK |
| | | | | ALPAPIEKTISKAKGQPREPQVYTLPPSREEMT |
| | | | | KNQVSLTCLVKGFYPSDIAVEWESNGQPENNY |
| | | | | KTTPPVLDSDGSFFLYSKLTVDKSRWQQGNVF |
| | | | | SCSVMHEALHNHYTQKSLSLSPGKGGGGQGG |
| | | | | GGQGGGGQGGGGQGGGGQGGGGQCPPCPAP |
| | | | | ELLGGPSVFLFPPKPKDTLMISRTPEVTCVVVD |
| | | | | VSHEEPEVKFNWYVDGVEVHNAKTKPCEEQY |
| | | | | GSTYRCVSVLTVLHQDWLNGKEYKCKVSNKA |
| | | | | LPAPIEKTISKAKGQPREPQVYTLPPSREEMTK |
| | | | | NQVSLTCLVKGFYPSDIAVEWESNGQPENNYK |
| | | | | TTPPVLDSDGSFFLYSKLTVDKSRWQQGNVFS |
| | | | | CSVMHEALHNHYTQKSLSLSPGKGGGGQVQL |
| | | | | VQSGAEVKKPGASVKVSCKASGYTFTSYWMH |
| | | | | WVRQAPGQCLEWIGYITPSTGYTEYNQKFKGR |
| | | | | VTMTRDKSTSTVYMELSSLTSEDTAVYYCARV |
| | | | | HDYDRAMEYWGQGTTVTVSSGGGGQGGGGQ |
| | | | | GGGGQEIQMTQSPSSLSASVGDRVTITCKASQD |
| | | | | INKYIAWYQQKPGKGPKLLIYYTSTLQPGVPSR |
| | | | | FSGSGSGTDFTFTISSLQPEDIATYYCLQYASYP |
| | | | | FTFGCGTRLEIKSGGGGQEVQLVESGGGLVQP |
| | | | | GGSLKLSCAASGFTFNKYAINWVRQAPGKGLE |
| | | | | WVARIRSKYNNYATYYADAVKDRFTISRDDSK |
| | | | | NTVYLQMNNLKTEDTAVYYCARAGNFGSSYIS |
| | | | | YWAYWGQGTLVTVSSGGGGQGGGGQGGGGQ |
| | | | | QTVVTQEPSLTVSPGGTVTITCGSSTGAVTSGN |
| | | | | YPNWVQKKPGQAPRGLIGGTKFLAPGTPARFS |
| | | | | GSLSGGKAALTLSGVQPEDEAEYYCVLWYSN |
| | | | | RWVFGSGTKLTVL |
| 376. | CL1 9-G4 CC - | artificial | Aa | DYYMH |
| | HCDR1 | | | |
| 377. | CL1 9-G4 CC - | artificial | Aa | WINPNSGGPNYAQKFQG |
| | HCDR2 | | | |
| 378. | CL1 9-G4 CC - | artificial | Aa | EKHAVAGIGFDY |
| | HCDR3 | | | |
| 379. | CL1 9-G4 CC - | artificial | Aa | QASQDISNYLN |
| | LCDR1 | | | |
| 380. | CL1 9-G4 CC - | artificial | Aa | AASSLES |
| | LCDR2 | | | |
| 381. | CL1 9-G4 CC - | artificial | aa | QQANSFPLT |
| | LCDR3 | | | |
| 382. | CL1 9-G4 CC - | artificial | aa | QVQLVQSGAEVKKPGASVKVSCKASGYTFTD |
| | VH | | | YYMHWVRQAPGQCLEWMGWINPNSGGPNYA |
| | | | | QKFQGRVTMTRDTSISTAHMELSRLRSDDTAV |
| | | | | YYCAREKHAVAGIGFDYWGQGTLVTVSS |
| 383. | CL1 9-G4 CC - | artificial | aa | DIQMTQSPSSVSASVGDRVTITCQASQDISNYL |
| | VL | | | NWYQQKPGKAPKLLIYAASSLESGVPSRFSGS |
| | | | | GSGTDFTLTISSLQPEDFATYYCQQANSFPLTFG |
| | | | | CGTKVDIK |
| 384. | CL1 9-G4 CC EI - | artificial | aa | EIQMTQSPSSVSASVGDRVTITCQASQDISNYL |
| | VL | | | NWYQQKPGKAPKLLIYAASSLESGVPSRFSGS |
| | | | | GSGTDFTLTISSLQPEDFATYYCQQANSFPLTFG |
| | | | | CGTKVDIK |
| 385. | FL 4-E9 CC - | artificial | aa | NARMGVS |
| | HCDR1 | | | |
| 386. | FL 4-E9 CC - | artificial | aa | HIFSNDEKSYSTSLKS |
| | HCDR2 | | | |
| 387. | FL 4-E9 CC - | artificial | aa | VPEYSSGWYRFDY |
| | HCDR3 | | | |
| 388. | FL 4-E9 CC - | artificial | aa | RASQSIRSYLN |
| | LCDR1 | | | |
| 389. | FL 4-E9 CC - | artificial | Aa | ATSSLQG |
| | LCDR2 | | | |
| 390. | FL 4-E9 CC - | artificial | Aa | QQSYSTPFT |
| | LCDR3 | | | |
| 391. | FL 4-E9 CC - VH | artificial | Aa | QVTLKESGPTLVKPTETLTLTCTVSGFSFRNAR |
| | | | | MGVSWIRQPPGKCLEWLAHIFSNDEKSYSTSL |
| | | | | KSRLTISKDTSKSQVVLTMTNMDPVDTATYFC |
| | | | | ARVPEYSSGWYRFDYWGQGTLVTVSS |
| 392. | FL 4-E9 CC - VL | artificial | Aa | DIQMTQSPSSLSASVGDRVTISCRASQSIRSYLN |
| | | | | WYQQKPGKAPKLLIYATSSLQGGVPSRFSGSG |
| | | | | SGTDFTLTISSLQPEDFATYYCQQSYSTPFTFGC |
| | | | | GTKVEIK |
| 393. | FL 4-E9 CC EI - | artificial | Aa | EIQMTQSPSSLSASVGDRVTISCRASQSIRSYLN |
| | VL | | | WYQQKPGKAPKLLIYATSSLQGGVPSRFSGSG |
| | | | | SGTDFTLTISSLQPEDFATYYCQQSYSTPFTFGC |
| | | | | GTKVEIK |
| 394. | CL1 9-G4 CC | artificial | Aa | QVQLVQSGAEVKKPGASVKVSCKASGYTFTD |
| | x4F10.03 scFc | | | YYMHWVRQAPGQCLEWMGWINPNSGGPNYA |
| | xFL 4-E9 CC | | | QKFQGRVTMTRDTSISTAHMELSRLRSDDTAV |
| | x4F10.03 mut - | | | YYCAREKHAVAGIGFDYWGQGTLVTVSSGGG |
| | Full Sequence | | | GSGGGGSGGGGSDIQMTQSPSSVSASVGDRVTI |
| | | | | TCQASQDISNYLNWYQQKPGKAPKLLIYAASS |
| | | | | LESGVPSRFSGSGSGTDFTLTISSLQPEDFATYY |
| | | | | CQQANSFPLTFGCGTKVDIKSGGGGSEVQLVE |
| | | | | SGGGLVQPGGSLKLSCAASGFTFNKYAMNWV |
| | | | | RQAPGKGMEWVARIRSKYNNYATYYADAVK |
| | | | | DRFTISRDDSKNTLYLQMNNLKTEDTAVYYCV |
| | | | | RAGNFGKSYISYWAYWGQGTLVTVSSGGGGS |
| | | | | GGGGSGGGGSQTVVTQEPSLTVSPGGTVTITC |
| | | | | GSSTGAVTSGNYPNWVQKKPGQAPRGLIGGTK |
| | | | | FLAPGTPARFSGSLSGGKAALTLSGVQPEDEAE |
| | | | | YYCVLWYSNRWVFGSGTKLTVLGGGGSGGG |
| | | | | GSGGGGSDKTHTCPPCPAPELLGGPSVFLFPPK |
| | | | | PKDTLMISRTPEVTCVVVDVSHEDPEVKFNWY |
| | | | | VDGVEVHNAKTKPCEEQYGSTYRCVSVLTVL |
| | | | | HQDWLNGKEYKCKVSNKALPAPIEKTISKAKG |
| | | | | QPREPQVYTLPPSREEMTKNQVSLTCLVKGFY |
| | | | | PSDIAVEWESNGQPENNYKTTPPVLDSDGSFFL |
| | | | | YSKLTVDKSRWQQGNVFSCSVMHEALHNHYT |
| | | | | QKSLSLSPGKGGGGSGGGGSGGGGSGGGGSG |
| | | | | GGGSGGGGSDKTHTCPPCPAPELLGGPSVFLFP |
| | | | | PKPKDTLMISRTPEVTCVVVDVSHEDPEVKEN |
| | | | | WYVDGVEVHNAKTKPCEEQYGSTYRCVSVLT |
| | | | | VLHQDWLNGKEYKCKVSNKALPAPIEKTISKA |
| | | | | KGQPREPQVYTLPPSREEMTKNQVSLTCLVKG |
| | | | | FYPSDIAVEWESNGQPENNYKTTPPVLDSDGSF |
| | | | | FLYSKLTVDKSRWQQGNVFSCSVMHEALHNH |
| | | | | YTQKSLSLSPGKGGGGSGGGGSGGGGSQVTLK |
| | | | | ESGPTLVKPTETLTLTCTVSGFSFRNARMGVS |
| | | | | WIRQPPGKCLEWLAHIFSNDEKSYSTSLKSRLTI |
| | | | | SKDTSKSQVVLTMTNMDPVDTATYFCARVPE |
| | | | | YSSGWYRFDYWGQGTLVTVSSGGGGSGGGGS |
| | | | | GGGGSDIQMTQSPSSLSASVGDRVTISCRASQSI |
| | | | | RSYLNWYQQKPGKAPKLLIYATSSLQGGVPSR |
| | | | | FSGSGSGTDFTLTISSLQPEDFATYYCQQSYSTP |
| | | | | FTFGCGTKVEIKSGGGGSEVQLVESGGGLVQP |
| | | | | GGSLKLSCAASGFTFNKYAMNWVRQAPGKG |
| | | | | MEWVARIRSKYNNYATYYADAVKDRFTISRD |
| | | | | DSKNTLYLQMNNLKTEDTAVYYCVRAGNFGK |
| | | | | SYISYWAYWGQGTLVTVSSGGGGSGGGGSGG |
| | | | | GGSQTVVTQEPSLTVSPGGTVTITCGSSTGAVT |
| | | | | SGNYPNWVQKKPGQAPRGLIGGTKFLAPGTPA |
| | | | | RFSGSLSGGKAALTLSGVQPEDEAEYYCVLWY |
| | | | | SNRWVFGSGTKLTVL |
| 395. | CL1 9-G4 CC | artificial | Aa | QVQLVQSGAEVKKPGASVKVSCKASGYTFTD |
| | x4G10.04x scFc | | | YYMHWVRQAPGQCLEWMGWINPNSGGPNYA |
| | xFL 4-E9 CC | | | QKFQGRVTMTRDTSISTAHMELSRLRSDDTAV |
| | x4G10.04 - Full | | | YYCAREKHAVAGIGFDYWGQGTLVTVSSGGG |
| | Sequence | | | GSGGGGSGGGGSDIQMTQSPSSVSASVGDRVTI |
| | | | | TCQASQDISNYLNWYQQKPGKAPKLLIYAASS |
| | | | | LESGVPSRFSGSGSGTDFTLTISSLQPEDFATYY |
| | | | | CQQANSFPLTFGCGTKVDIKSGGGGSEVQLVE |
| | | | | SGGGLVQPGGSLKLSCAASGFTFSKYAMNWV |
| | | | | REAPGKGLEWVARIRSKYNNYATYYAEAVKD |
| | | | | RFTISRDDSKNTVYLQMNNLKTEDTAVYYCVR |
| | | | | AENIGKSYISYWAYWGQGTLVTVSSGGGGSG |
| | | | | GGGSGGGGSQTVVTQEPSLTVSPGGTVTMTCG |
| | | | | SSTGAVTSGNYPNWVQKKPGQAPRGLIGGTKF |
| | | | | LAPGTPARFSGSLEGGKAALTLSGVQPEDEAE |
| | | | | YYCVLWYSNRWVFGGGTKLTVLGGGGSGGG |
| | | | | GSGGGGSDKTHTCPPCPAPELLGGPSVFLFPPK |
| | | | | PKDTLMISRTPEVTCVVVDVSHEDPEVKFNWY |
| | | | | VDGVEVHNAKTKPCEEQYGSTYRCVSVLTVL |
| | | | | HQDWLNGKEYKCKVSNKALPAPIEKTISKAKG |
| | | | | QPREPQVYTLPPSREEMTKNQVSLTCLVKGFY |
| | | | | PSDIAVEWESNGQPENNYKTTPPVLDSDGSFFL |
| | | | | YSKLTVDKSRWQQGNVFSCSVMHEALHNHYT |
| | | | | QKSLSLSPGKGGGGSGGGGSGGGGSGGGGSG |
| | | | | GGGSGGGGSDKTHTCPPCPAPELLGGPSVFLFP |
| | | | | PKPKDTLMISRTPEVTCVVVDVSHEDPEVKEN |
| | | | | WYVDGVEVHNAKTKPCEEQYGSTYRCVSVLT |
| | | | | VLHQDWLNGKEYKCKVSNKALPAPIEKTISKA |
| | | | | KGQPREPQVYTLPPSREEMTKNQVSLTCLVKG |
| | | | | FYPSDIAVEWESNGQPENNYKTTPPVLDSDGSF |
| | | | | FLYSKLTVDKSRWQQGNVFSCSVMHEALHNH |
| | | | | YTQKSLSLSPGKGGGGSGGGGSGGGGSQVTLK |
| | | | | ESGPTLVKPTETLTLTCTVSGFSFRNARMGVS |
| | | | | WIRQPPGKCLEWLAHIFSNDEKSYSTSLKSRLTI |
| | | | | SKDTSKSQVVLTMTNMDPVDTATYFCARVPE |
| | | | | YSSGWYRFDYWGQGTLVTVSSGGGGSGGGGS |
| | | | | GGGGSDIQMTQSPSSLSASVGDRVTISCRASQSI |
| | | | | RSYLNWYQQKPGKAPKLLIYATSSLQGGVPSR |
| | | | | FSGSGSGTDFTLTISSLQPEDFATYYCQQSYSTP |
| | | | | FTFGCGTKVEIKSGGGGSEVQLVESGGGLVQP |
| | | | | GGSLKLSCAASGFTFSKYAMNWVREAPGKGL |
| | | | | EWVARIRSKYNNYATYYAEAVKDRFTISRDDS |
| | | | | KNTVYLQMNNLKTEDTAVYYCVRAENIGKSYI |
| | | | | SYWAYWGQGTLVTVSSGGGGSGGGGSGGGG |
| | | | | SQTVVTQEPSLTVSPGGTVTMTCGSSTGAVTS |
| | | | | GNYPNWVQKKPGQAPRGLIGGTKFLAPGTPAR |
| | | | | FSGSLEGGKAALTLSGVQPEDEAEYYCVLWYS |
| | | | | NRWVFGGGTKLTVL |
| 396. | CL1 9-G4 CC | artificial | Aa | QVQLVQSGAEVKKPGASVKVSCKASGYTFTD |
| | x5B1.05 x scFc | | | YYMHWVRQAPGQCLEWMGWINPNSGGPNYA |
| | xFL 4-E9 CC | | | QKFQGRVTMTRDTSISTAHMELSRLRSDDTAV |
| | x5B1.05 - Full | | | YYCAREKHAVAGIGFDYWGQGTLVTVSSGGG |
| | Sequence | | | GSGGGGSGGGGSDIQMTQSPSSVSASVGDRVTI |
| | | | | TCQASQDISNYLNWYQQKPGKAPKLLIYAASS |
| | | | | LESGVPSRFSGSGSGTDFTLTISSLQPEDFATYY |
| | | | | CQQANSFPLTFGCGTKVDIKSGGGGSEVQLVE |
| | | | | SGGGLVQPGGSLKLSCAASGFTFSKYAMNWV |
| | | | | RQAPGKGMEWVARIRSKYNNYATYYAEAVK |
| | | | | GRFTISRDDSKNTVYLQMNNLKTEDTAVYYCV |
| | | | | RAGNFGSSYISYWAYWGQGTLVTVSSGGGGS |
| | | | | GGGGSGGGGSQTVVTQEPSLTVSPGGTVTLTC |
| | | | | GSSTGAVTSGNYPNWVQKKPGQAPRGLIGGTK |
| | | | | FLAPGTPARFSGSLSGGKAALTLSGVQPEDEAE |
| | | | | YYCVLWYSNRWVFGGGTKLTVLGGGGSGGG |
| | | | | GSGGGGSDKTHTCPPCPAPELLGGPSVFLFPPK |
| | | | | PKDTLMISRTPEVTCVVVDVSHEDPEVKFNWY |
| | | | | VDGVEVHNAKTKPCEEQYGSTYRCVSVLTVL |
| | | | | HQDWLNGKEYKCKVSNKALPAPIEKTISKAKG |
| | | | | QPREPQVYTLPPSREEMTKNQVSLTCLVKGFY |
| | | | | PSDIAVEWESNGQPENNYKTTPPVLDSDGSFFL |
| | | | | YSKLTVDKSRWQQGNVFSCSVMHEALHNHYT |
| | | | | QKSLSLSPGKGGGGSGGGGSGGGGSGGGGSG |
| | | | | GGGSGGGGSDKTHTCPPCPAPELLGGPSVFLFP |
| | | | | PKPKDTLMISRTPEVTCVVVDVSHEDPEVKEN |
| | | | | WYVDGVEVHNAKTKPCEEQYGSTYRCVSVLT |
| | | | | VLHQDWLNGKEYKCKVSNKALPAPIEKTISKA |
| | | | | KGQPREPQVYTLPPSREEMTKNQVSLTCLVKG |
| | | | | FYPSDIAVEWESNGQPENNYKTTPPVLDSDGSF |
| | | | | FLYSKLTVDKSRWQQGNVFSCSVMHEALHNH |
| | | | | YTQKSLSLSPGKGGGGSGGGGSGGGGSQVTLK |
| | | | | ESGPTLVKPTETLTLTCTVSGFSFRNARMGVS |
| | | | | WIRQPPGKCLEWLAHIFSNDEKSYSTSLKSRLTI |
| | | | | SKDTSKSQVVLTMTNMDPVDTATYFCARVPE |
| | | | | YSSGWYRFDYWGQGTLVTVSSGGGGSGGGGS |
| | | | | GGGGSDIQMTQSPSSLSASVGDRVTISCRASQSI |
| | | | | RSYLNWYQQKPGKAPKLLIYATSSLQGGVPSR |
| | | | | FSGSGSGTDFTLTISSLQPEDFATYYCQQSYSTP |
| | | | | FTFGCGTKVEIKSGGGGSEVQLVESGGGLVQP |
| | | | | GGSLKLSCAASGFTFSKYAMNWVRQAPGKGM |
| | | | | EWVARIRSKYNNYATYYAEAVKGRFTISRDDS |
| | | | | KNTVYLQMNNLKTEDTAVYYCVRAGNFGSSY |
| | | | | ISYWAYWGQGTLVTVSSGGGGSGGGGSGGGG |
| | | | | SQTVVTQEPSLTVSPGGTVTLTCGSSTGAVTSG |
| | | | | NYPNWVQKKPGQAPRGLIGGTKFLAPGTPARF |
| | | | | SGSLSGGKAALTLSGVQPEDEAEYYCVLWYSN |
| | | | | RWVFGGGTKLTVL |
| 397. | CL1 9-G4 CC | artificial | Aa | QVQLVQSGAEVKKPGASVKVSCKASGYTFTD |
| | x5B1.09 x scFc | | | YYMHWVRQAPGQCLEWMGWINPNSGGPNYA |
| | xFL 4-E9 CC | | | QKFQGRVTMTRDTSISTAHMELSRLRSDDTAV |
| | xH5B1.09 - Full | | | YYCAREKHAVAGIGFDYWGQGTLVTVSSGGG |
| | Sequence | | | GSGGGGSGGGGSDIQMTQSPSSVSASVGDRVTI |
| | | | | TCQASQDISNYLNWYQQKPGKAPKLLIYAASS |
| | | | | LESGVPSRFSGSGSGTDFTLTISSLQPEDFATYY |
| | | | | CQQANSFPLTFGCGTKVDIKSGGGGSEVQLVE |
| | | | | SGGGLVQPGGSLKLSCAASGFTFSKYAMNWV |
| | | | | RQAPGKGMEWVARIRSKYNNYATYYADAVK |
| | | | | GRFTISRDDSKNTVYLQMNNLKTEDTAVYYCV |
| | | | | RAGNFGKSYISYFAYWGQGTLVTVSSGGGGSG |
| | | | | GGGSGGGGSQTVVTQEPSLTVSPGGTVTLTCG |
| | | | | SSTGAVTSGNYPNWVQKKPGQAPRGLIGGTKF |
| | | | | LAPGTPARFSGSLSGGKAALTLSGVQPEDEAEY |
| | | | | YCVLYYSNRWVFGGGTKLTVLGGGGSGGGGS |
| | | | | GGGGSDKTHTCPPCPAPELLGGPSVFLFPPKPK |
| | | | | DTLMISRTPEVTCVVVDVSHEDPEVKFNWYVD |
| | | | | GVEVHNAKTKPCEEQYGSTYRCVSVLTVLHQ |
| | | | | DWLNGKEYKCKVSNKALPAPIEKTISKAKGQP |
| | | | | REPQVYTLPPSREEMTKNQVSLTCLVKGFYPS |
| | | | | DIAVEWESNGQPENNYKTTPPVLDSDGSFFLYS |
| | | | | KLTVDKSRWQQGNVFSCSVMHEALHNHYTQK |
| | | | | SLSLSPGKGGGGSGGGGSGGGGSGGGGSGGG |
| | | | | GSGGGGSDKTHTCPPCPAPELLGGPSVFLFPPK |
| | | | | PKDTLMISRTPEVTCVVVDVSHEDPEVKFNWY |
| | | | | VDGVEVHNAKTKPCEEQYGSTYRCVSVLTVL |
| | | | | HQDWLNGKEYKCKVSNKALPAPIEKTISKAKG |
| | | | | QPREPQVYTLPPSREEMTKNQVSLTCLVKGFY |
| | | | | PSDIAVEWESNGQPENNYKTTPPVLDSDGSFFL |
| | | | | YSKLTVDKSRWQQGNVFSCSVMHEALHNHYT |
| | | | | QKSLSLSPGKGGGGSGGGGSGGGGSQVTLKES |
| | | | | GPTLVKPTETLTLTCTVSGFSFRNARMGVSWIR |
| | | | | QPPGKCLEWLAHIFSNDEKSYSTSLKSRLTISK |
| | | | | DTSKSQVVLTMTNMDPVDTATYFCARVPEYSS |
| | | | | GWYRFDYWGQGTLVTVSSGGGGSGGGGSGG |
| | | | | GGSDIQMTQSPSSLSASVGDRVTISCRASQSIRS |
| | | | | YLNWYQQKPGKAPKLLIYATSSLQGGVPSRFS |
| | | | | GSGSGTDFTLTISSLQPEDFATYYCQQSYSTPFT |
| | | | | FGCGTKVEIKSGGGGSEVQLVESGGGLVQPGG |
| | | | | SLKLSCAASGFTFSKYAMNWVRQAPGKGMEW |
| | | | | VARIRSKYNNYATYYADAVKGRFTISRDDSKN |
| | | | | TVYLQMNNLKTEDTAVYYCVRAGNFGKSYIS |
| | | | | YFAYWGQGTLVTVSSGGGGSGGGGSGGGGSQ |
| | | | | TVVTQEPSLTVSPGGTVTLTCGSSTGAVTSGNY |
| | | | | PNWVQKKPGQAPRGLIGGTKFLAPGTPARFSG |
| | | | | SLSGGKAALTLSGVQPEDEAEYYCVLYYSNR |
| | | | | WVFGGGTKLTVL |
| 398. | CL1 9-G4 CC | artificial | aa | QVQLVQSGAEVKKPGASVKVSCKASGYTFTD |
| | x6H10.03x scFc | | | YYMHWVRQAPGQCLEWMGWINPNSGGPNYA |
| | xFL 4-E9 CC x | | | QKFQGRVTMTRDTSISTAHMELSRLRSDDTAV |
| | 6H10.03 - Full | | | YYCAREKHAVAGIGFDYWGQGTLVTVSSGGG |
| | Sequence | | | GSGGGGSGGGGSDIQMTQSPSSVSASVGDRVTI |
| | | | | TCQASQDISNYLNWYQQKPGKAPKLLIYAASS |
| | | | | LESGVPSRFSGSGSGTDFTLTISSLQPEDFATYY |
| | | | | CQQANSFPLTFGCGTKVDIKSGGGGSEVQLVE |
| | | | | SGGGLVQPGGSLKLSCAASGFTFNKYAMNWV |
| | | | | RQAPGKGMEWVARIRSKYNNYATYYAEAVK |
| | | | | DRFTISRDDSKNTLYLQMNNLKTEDTAVYYCV |
| | | | | RAGNFGKSYISYWAYWGQGTLVTVSSGGGGS |
| | | | | GGGGSGGGGSQTVVTQEPSLTVSPGGTVTITC |
| | | | | GSSTGAVTSGNYPNWIQKKPGQAPRGLIGGTK |
| | | | | FLAPGTPARFSGSLEGGKAALTLSGVQPEDEAE |
| | | | | YYCVLWYSNRWVFGSGTKLTVLGGGGSGGG |
| | | | | GSGGGGSDKTHTCPPCPAPELLGGPSVFLFPPK |
| | | | | PKDTLMISRTPEVTCVVVDVSHEDPEVKFNWY |
| | | | | VDGVEVHNAKTKPCEEQYGSTYRCVSVLTVL |
| | | | | HQDWLNGKEYKCKVSNKALPAPIEKTISKAKG |
| | | | | QPREPQVYTLPPSREEMTKNQVSLTCLVKGFY |
| | | | | PSDIAVEWESNGQPENNYKTTPPVLDSDGSFFL |
| | | | | YSKLTVDKSRWQQGNVFSCSVMHEALHNHYT |
| | | | | QKSLSLSPGKGGGGSGGGGSGGGGSGGGGSG |
| | | | | GGGSGGGGSDKTHTCPPCPAPELLGGPSVFLFP |
| | | | | PKPKDTLMISRTPEVTCVVVDVSHEDPEVKEN |
| | | | | WYVDGVEVHNAKTKPCEEQYGSTYRCVSVLT |
| | | | | VLHQDWLNGKEYKCKVSNKALPAPIEKTISKA |
| | | | | KGQPREPQVYTLPPSREEMTKNQVSLTCLVKG |
| | | | | FYPSDIAVEWESNGQPENNYKTTPPVLDSDGSF |
| | | | | FLYSKLTVDKSRWQQGNVFSCSVMHEALHNH |
| | | | | YTQKSLSLSPGKGGGGSGGGGSGGGGSQVTLK |
| | | | | ESGPTLVKPTETLTLTCTVSGFSFRNARMGVS |
| | | | | WIRQPPGKCLEWLAHIFSNDEKSYSTSLKSRLTI |
| | | | | SKDTSKSQVVLTMTNMDPVDTATYFCARVPE |
| | | | | YSSGWYRFDYWGQGTLVTVSSGGGGSGGGGS |
| | | | | GGGGSDIQMTQSPSSLSASVGDRVTISCRASQSI |
| | | | | RSYLNWYQQKPGKAPKLLIYATSSLQGGVPSR |
| | | | | FSGSGSGTDFTLTISSLQPEDFATYYCQQSYSTP |
| | | | | FTFGCGTKVEIKSGGGGSEVQLVESGGGLVQP |
| | | | | GGSLKLSCAASGFTFNKYAMNWVRQAPGKG |
| | | | | MEWVARIRSKYNNYATYYAEAVKDRFTISRD |
| | | | | DSKNTLYLQMNNLKTEDTAVYYCVRAGNFGK |
| | | | | SYISYWAYWGQGTLVTVSSGGGGSGGGGSGG |
| | | | | GGSQTVVTQEPSLTVSPGGTVTITCGSSTGAVT |
| | | | | SGNYPNWIQKKPGQAPRGLIGGTKFLAPGTPA |
| | | | | RFSGSLEGGKAALTLSGVQPEDEAEYYCVLWY |
| | | | | SNRWVFGSGTKLTVL |
| 399. | CL1 9-G4 CC | artificial | aa | QVQLVQSGAEVKKPGASVKVSCKASGYTFTD |
| | x6H10.09 x scFc | | | YYMHWVRQAPGQCLEWMGWINPNSGGPNYA |
| | xFL 4-E9 CC | | | QKFQGRVTMTRDTSISTAHMELSRLRSDDTAV |
| | x6H10.09 - Full | | | YYCAREKHAVAGIGFDYWGQGTLVTVSSGGG |
| | Sequence | | | GSGGGGSGGGGSDIQMTQSPSSVSASVGDRVTI |
| | | | | TCQASQDISNYLNWYQQKPGKAPKLLIYAASS |
| | | | | LESGVPSRFSGSGSGTDFTLTISSLQPEDFATYY |
| | | | | CQQANSFPLTFGCGTKVDIKSGGGGSEVQLVE |
| | | | | SGGGLVQPGGSLKLSCAASGFTFNKYAMNWV |
| | | | | RQAPGKGMEWVARIRSKYNNYATYYADAVK |
| | | | | DRFTISRDDSKNTLYLQMNNLKTEDTAVYYCV |
| | | | | RAGNFGSSYISYFAYWGQGTLVTVSSGGGGSG |
| | | | | GGGSGGGGSQTVVTQEPSLTVSPGGTVTITCGS |
| | | | | STGAVTSGNYPNWIQKKPGQAPRGLIGGTKFL |
| | | | | APGTPARFSGSLEGGKAALTLSGVQPEDEAEY |
| | | | | YCVLYYSNRWVFGSGTKLTVLGGGGSGGGGS |
| | | | | GGGGSDKTHTCPPCPAPELLGGPSVFLFPPKPK |
| | | | | DTLMISRTPEVTCVVVDVSHEDPEVKFNWYVD |
| | | | | GVEVHNAKTKPCEEQYGSTYRCVSVLTVLHQ |
| | | | | DWLNGKEYKCKVSNKALPAPIEKTISKAKGQP |
| | | | | REPQVYTLPPSREEMTKNQVSLTCLVKGFYPS |
| | | | | DIAVEWESNGQPENNYKTTPPVLDSDGSFFLYS |
| | | | | KLTVDKSRWQQGNVFSCSVMHEALHNHYTQK |
| | | | | SLSLSPGKGGGGSGGGGSGGGGSGGGGSGGG |
| | | | | GSGGGGSDKTHTCPPCPAPELLGGPSVFLFPPK |
| | | | | PKDTLMISRTPEVTCVVVDVSHEDPEVKFNWY |
| | | | | VDGVEVHNAKTKPCEEQYGSTYRCVSVLTVL |
| | | | | HQDWLNGKEYKCKVSNKALPAPIEKTISKAKG |
| | | | | QPREPQVYTLPPSREEMTKNQVSLTCLVKGFY |
| | | | | PSDIAVEWESNGQPENNYKTTPPVLDSDGSFFL |
| | | | | YSKLTVDKSRWQQGNVFSCSVMHEALHNHYT |
| | | | | QKSLSLSPGKGGGGSGGGGSGGGGSQVTLKES |
| | | | | GPTLVKPTETLTLTCTVSGFSFRNARMGVSWIR |
| | | | | QPPGKCLEWLAHIFSNDEKSYSTSLKSRLTISK |
| | | | | DTSKSQVVLTMTNMDPVDTATYFCARVPEYSS |
| | | | | GWYRFDYWGQGTLVTVSSGGGGSGGGGSGG |
| | | | | GGSDIQMTQSPSSLSASVGDRVTISCRASQSIRS |
| | | | | YLNWYQQKPGKAPKLLIYATSSLQGGVPSRFS |
| | | | | GSGSGTDFTLTISSLQPEDFATYYCQQSYSTPFT |
| | | | | FGCGTKVEIKSGGGGSEVQLVESGGGLVQPGG |
| | | | | SLKLSCAASGFTFNKYAMNWVRQAPGKGME |
| | | | | WVARIRSKYNNYATYYADAVKDRFTISRDDSK |
| | | | | NTLYLQMNNLKTEDTAVYYCVRAGNFGSSYIS |
| | | | | YFAYWGQGTLVTVSSGGGGSGGGGSGGGGSQ |
| | | | | TVVTQEPSLTVSPGGTVTITCGSSTGAVTSGNY |
| | | | | PNWIQKKPGQAPRGLIGGTKFLAPGTPARFSGS |
| | | | | LEGGKAALTLSGVQPEDEAEYYCVLYYSNRW |
| | | | | VFGSGTKLTVL |
| 400. | CL1 9-G4 CC x | artificial | aa | QVQLVQSGAEVKKPGASVKVSCKASGYTFTD |
| | I2C x scFc x I2C | | | YYMHWVRQAPGQCLEWMGWINPNSGGPNYA |
| | xFL 4-E9 CC - | | | QKFQGRVTMTRDTSISTAHMELSRLRSDDTAV |
| | Full Sequence | | | YYCAREKHAVAGIGFDYWGQGTLVTVSSGGG |
| | | | | GSGGGGSGGGGSDIQMTQSPSSVSASVGDRVTI |
| | | | | TCQASQDISNYLNWYQQKPGKAPKLLIYAASS |
| | | | | LESGVPSRFSGSGSGTDFTLTISSLQPEDFATYY |
| | | | | CQQANSFPLTFGCGTKVDIKSGGGGSEVQLVE |
| | | | | SGGGLVQPGGSLKLSCAASGFTFNKYAMNWV |
| | | | | RQAPGKGLEWVARIRSKYNNYATYYADSVKD |
| | | | | RFTISRDDSKNTAYLQMNNLKTEDTAVYYCVR |
| | | | | HGNFGNSYISYWAYWGQGTLVTVSSGGGGSG |
| | | | | GGGSGGGGSQTVVTQEPSLTVSPGGTVTLTCG |
| | | | | SSTGAVTSGNYPNWVQQKPGQAPRGLIGGTKF |
| | | | | LAPGTPARFSGSLLGGKAALTLSGVQPEDEAE |
| | | | | YYCVLWYSNRWVFGGGTKLTVLGGGGSGGG |
| | | | | GSGGGGSDKTHTCPPCPAPELLGGPSVFLFPPK |
| | | | | PKDTLMISRTPEVTCVVVDVSHEDPEVKFNWY |
| | | | | VDGVEVHNAKTKPCEEQYGSTYRCVSVLTVL |
| | | | | HQDWLNGKEYKCKVSNKALPAPIEKTISKAKG |
| | | | | QPREPQVYTLPPSREEMTKNQVSLTCLVKGFY |
| | | | | PSDIAVEWESNGQPENNYKTTPPVLDSDGSFFL |
| | | | | YSKLTVDKSRWQQGNVFSCSVMHEALHNHYT |
| | | | | QKSLSLSPGKGGGGSGGGGSGGGGSGGGGSG |
| | | | | GGGSGGGGSDKTHTCPPCPAPELLGGPSVFLFP |
| | | | | PKPKDTLMISRTPEVTCVVVDVSHEDPEVKEN |
| | | | | WYVDGVEVHNAKTKPCEEQYGSTYRCVSVLT |
| | | | | VLHQDWLNGKEYKCKVSNKALPAPIEKTISKA |
| | | | | KGQPREPQVYTLPPSREEMTKNQVSLTCLVKG |
| | | | | FYPSDIAVEWESNGQPENNYKTTPPVLDSDGSF |
| | | | | FLYSKLTVDKSRWQQGNVFSCSVMHEALHNH |
| | | | | YTQKSLSLSPGKGGGGSGGGGSGGGGSEVQLV |
| | | | | ESGGGLVQPGGSLKLSCAASGFTFNKYAMNW |
| | | | | VRQAPGKGLEWVARIRSKYNNYATYYADSVK |
| | | | | DRFTISRDDSKNTAYLQMNNLKTEDTAVYYCV |
| | | | | RHGNFGNSYISYWAYWGQGTLVTVSSGGGGS |
| | | | | GGGGSGGGGSQTVVTQEPSLTVSPGGTVTLTC |
| | | | | GSSTGAVTSGNYPNWVQQKPGQAPRGLIGGTK |
| | | | | FLAPGTPARFSGSLLGGKAALTLSGVQPEDEAE |
| | | | | YYCVLWYSNRWVFGGGTKLTVLGGGGSQVTL |
| | | | | KESGPTLVKPTETLTLTCTVSGFSFRNARMGVS |
| | | | | WIRQPPGKCLEWLAHIFSNDEKSYSTSLKSRLTI |
| | | | | SKDTSKSQVVLTMTNMDPVDTATYFCARVPE |
| | | | | YSSGWYRFDYWGQGTLVTVSSGGGGSGGGGS |
| | | | | GGGGSDIQMTQSPSSLSASVGDRVTISCRASQSI |
| | | | | RSYLNWYQQKPGKAPKLLIYATSSLQGGVPSR |
| | | | | FSGSGSGTDFTLTISSLQPEDFATYYCQQSYSTP |
| | | | | FTFGCGTKVEIK |
| 401. | CL1 9-G4 CC x | artificial | aa | QVQLVQSGAEVKKPGASVKVSCKASGYTFTD |
| | I2C x(G4S)3xscFc | | | YYMHWVRQAPGQCLEWMGWINPNSGGPNYA |
| | x(G4S)3 xFL 4-E9 | | | QKFQGRVTMTRDTSISTAHMELSRLRSDDTAV |
| | CC x I2C - Full | | | YYCAREKHAVAGIGFDYWGQGTLVTVSSGGG |
| | Sequence | | | GSGGGGSGGGGSDIQMTQSPSSVSASVGDRVTI |
| | | | | TCQASQDISNYLNWYQQKPGKAPKLLIYAASS |
| | | | | LESGVPSRFSGSGSGTDFTLTISSLQPEDFATYY |
| | | | | CQQANSFPLTFGCGTKVDIKSGGGGSEVQLVE |
| | | | | SGGGLVQPGGSLKLSCAASGFTFNKYAMNWV |
| | | | | RQAPGKGLEWVARIRSKYNNYATYYADSVKD |
| | | | | RFTISRDDSKNTAYLQMNNLKTEDTAVYYCVR |
| | | | | HGNFGNSYISYWAYWGQGTLVTVSSGGGGSG |
| | | | | GGGSGGGGSQTVVTQEPSLTVSPGGTVTLTCG |
| | | | | SSTGAVTSGNYPNWVQQKPGQAPRGLIGGTKF |
| | | | | LAPGTPARFSGSLLGGKAALTLSGVQPEDEAE |
| | | | | YYCVLWYSNRWVFGGGTKLTVLGGGGSGGG |
| | | | | GSGGGGSDKTHTCPPCPAPELLGGPSVFLFPPK |
| | | | | PKDTLMISRTPEVTCVVVDVSHEDPEVKFNWY |
| | | | | VDGVEVHNAKTKPCEEQYGSTYRCVSVLTVL |
| | | | | HQDWLNGKEYKCKVSNKALPAPIEKTISKAKG |
| | | | | QPREPQVYTLPPSREEMTKNQVSLTCLVKGFY |
| | | | | PSDIAVEWESNGQPENNYKTTPPVLDSDGSFFL |
| | | | | YSKLTVDKSRWQQGNVFSCSVMHEALHNHYT |
| | | | | QKSLSLSPGKGGGGSGGGGSGGGGSGGGGSG |
| | | | | GGGSGGGGSDKTHTCPPCPAPELLGGPSVFLFP |
| | | | | PKPKDTLMISRTPEVTCVVVDVSHEDPEVKFN |
| | | | | WYVDGVEVHNAKTKPCEEQYGSTYRCVSVLT |
| | | | | VLHQDWLNGKEYKCKVSNKALPAPIEKTISKA |
| | | | | KGQPREPQVYTLPPSREEMTKNQVSLTCLVKG |
| | | | | FYPSDIAVEWESNGQPENNYKTTPPVLDSDGSF |
| | | | | FLYSKLTVDKSRWQQGNVFSCSVMHEALHNH |
| | | | | YTQKSLSLSPGKGGGGSGGGGSGGGGSQVTLK |
| | | | | ESGPTLVKPTETLTLTCTVSGFSFRNARMGVS |
| | | | | WIRQPPGKCLEWLAHIFSNDEKSYSTSLKSRLTI |
| | | | | SKDTSKSQVVLTMTNMDPVDTATYFCARVPE |
| | | | | YSSGWYRFDYWGQGTLVTVSSGGGGSGGGGS |
| | | | | GGGGSDIQMTQSPSSLSASVGDRVTISCRASQSI |
| | | | | RSYLNWYQQKPGKAPKLLIYATSSLQGGVPSR |
| | | | | FSGSGSGTDFTLTISSLQPEDFATYYCQQSYSTP |
| | | | | FTFGCGTKVEIKSGGGGSEVQLVESGGGLVQP |
| | | | | GGSLKLSCAASGFTFNKYAMNWVRQAPGKGL |
| | | | | EWVARIRSKYNNYATYYADSVKDRFTISRDDS |
| | | | | KNTAYLQMNNLKTEDTAVYYCVRHGNFGNSY |
| | | | | ISYWAYWGQGTLVTVSSGGGGSGGGGSGGGG |
| | | | | SQTVVTQEPSLTVSPGGTVTLTCGSSTGAVTSG |
| | | | | NYPNWVQQKPGQAPRGLIGGTKFLAPGTPARF |
| | | | | SGSLLGGKAALTLSGVQPEDEAEYYCVLWYSN |
| | | | | RWVFGGGTKLTVL |
| 402. | CL1 9-G4 CC x | artificial | aa | QVQLVQSGAEVKKPGASVKVSCKASGYTFTD |
| | I2Ccc x scFc xFL | | | YYMHWVRQAPGQCLEWMGWINPNSGGPNYA |
| | 4-E9 CC x I2Ccc - | | | QKFQGRVTMTRDTSISTAHMELSRLRSDDTAV |
| | Full Sequence | | | YYCAREKHAVAGIGFDYWGQGTLVTVSSGGG |
| | | | | GSGGGGSGGGGSDIQMTQSPSSVSASVGDRVTI |
| | | | | TCQASQDISNYLNWYQQKPGKAPKLLIYAASS |
| | | | | LESGVPSRFSGSGSGTDFTLTISSLQPEDFATYY |
| | | | | CQQANSFPLTFGCGTKVDIKSGGGGSEVQLVE |
| | | | | SGGGLVQPGGSLKLSCAASGFTFNKYAMNWV |
| | | | | RQAPGKCLEWVARIRSKYNNYATYYADSVKD |
| | | | | RFTISRDDSKNTAYLQMNNLKTEDTAVYYCVR |
| | | | | HGNFGNSYISYWAYWGQGTLVTVSSGGGGSG |
| | | | | GGGSGGGGSQTVVTQEPSLTVSPGGTVTLTCG |
| | | | | SSTGAVTSGNYPNWVQQKPGQAPRGLIGGTKF |
| | | | | LAPGTPARFSGSLLGGKAALTLSGVQPEDEAE |
| | | | | YYCVLWYSNRWVFGCGTKLTVLGGGGSGGG |
| | | | | GSGGGGSDKTHTCPPCPAPELLGGPSVFLFPPK |
| | | | | PKDTLMISRTPEVTCVVVDVSHEDPEVKFNWY |
| | | | | VDGVEVHNAKTKPCEEQYGSTYRCVSVLTVL |
| | | | | HQDWLNGKEYKCKVSNKALPAPIEKTISKAKG |
| | | | | QPREPQVYTLPPSREEMTKNQVSLTCLVKGFY |
| | | | | PSDIAVEWESNGQPENNYKTTPPVLDSDGSFFL |
| | | | | YSKLTVDKSRWQQGNVFSCSVMHEALHNHYT |
| | | | | QKSLSLSPGKGGGGSGGGGSGGGGSGGGGSG |
| | | | | GGGSGGGGSDKTHTCPPCPAPELLGGPSVFLFP |
| | | | | PKPKDTLMISRTPEVTCVVVDVSHEDPEVKEN |
| | | | | WYVDGVEVHNAKTKPCEEQYGSTYRCVSVLT |
| | | | | VLHQDWLNGKEYKCKVSNKALPAPIEKTISKA |
| | | | | KGQPREPQVYTLPPSREEMTKNQVSLTCLVKG |
| | | | | FYPSDIAVEWESNGQPENNYKTTPPVLDSDGSF |
| | | | | FLYSKLTVDKSRWQQGNVFSCSVMHEALHNH |
| | | | | YTQKSLSLSPGKGGGGSGGGGSGGGGSQVTLK |
| | | | | ESGPTLVKPTETLTLTCTVSGFSFRNARMGVS |
| | | | | WIRQPPGKCLEWLAHIFSNDEKSYSTSLKSRLTI |
| | | | | SKDTSKSQVVLTMTNMDPVDTATYFCARVPE |
| | | | | YSSGWYRFDYWGQGTLVTVSSGGGGSGGGGS |
| | | | | GGGGSDIQMTQSPSSLSASVGDRVTISCRASQSI |
| | | | | RSYLNWYQQKPGKAPKLLIYATSSLQGGVPSR |
| | | | | FSGSGSGTDFTLTISSLQPEDFATYYCQQSYSTP |
| | | | | FTFGCGTKVEIKSGGGGSEVQLVESGGGLVQP |
| | | | | GGSLKLSCAASGFTFNKYAMNWVRQAPGKCL |
| | | | | EWVARIRSKYNNYATYYADSVKDRFTISRDDS |
| | | | | KNTAYLQMNNLKTEDTAVYYCVRHGNFGNSY |
| | | | | ISYWAYWGQGTLVTVSSGGGGSGGGGSGGGG |
| | | | | SQTVVTQEPSLTVSPGGTVTLTCGSSTGAVTSG |
| | | | | NYPNWVQQKPGQAPRGLIGGTKFLAPGTPARF |
| | | | | SGSLLGGKAALTLSGVQPEDEAEYYCVLWYSN |
| | | | | RWVFGCGTKLTVL |
| 403. | CL1 9-G4 CC x | artificial | aa | QVQLVQSGAEVKKPGASVKVSCKASGYTFTD |
| | I2Ccc x scFc x | | | YYMHWVRQAPGQCLEWMGWINPNSGGPNYA |
| | I2Ccc xFL 4-E9 | | | QKFQGRVTMTRDTSISTAHMELSRLRSDDTAV |
| | CC - Full | | | YYCAREKHAVAGIGFDYWGQGTLVTVSSGGG |
| | Sequence | | | GSGGGGSGGGGSDIQMTQSPSSVSASVGDRVTI |
| | | | | TCQASQDISNYLNWYQQKPGKAPKLLIYAASS |
| | | | | LESGVPSRFSGSGSGTDFTLTISSLQPEDFATYY |
| | | | | CQQANSFPLTFGCGTKVDIKSGGGGSEVQLVE |
| | | | | SGGGLVQPGGSLKLSCAASGFTFNKYAMNWV |
| | | | | RQAPGKCLEWVARIRSKYNNYATYYADSVKD |
| | | | | RFTISRDDSKNTAYLQMNNLKTEDTAVYYCVR |
| | | | | HGNFGNSYISYWAYWGQGTLVTVSSGGGGSG |
| | | | | GGGSGGGGSQTVVTQEPSLTVSPGGTVTLTCG |
| | | | | SSTGAVTSGNYPNWVQQKPGQAPRGLIGGTKF |
| | | | | LAPGTPARFSGSLLGGKAALTLSGVQPEDEAE |
| | | | | YYCVLWYSNRWVFGCGTKLTVLGGGGSGGG |
| | | | | GSGGGGSDKTHTCPPCPAPELLGGPSVFLFPPK |
| | | | | PKDTLMISRTPEVTCVVVDVSHEDPEVKFNWY |
| | | | | VDGVEVHNAKTKPCEEQYGSTYRCVSVLTVL |
| | | | | HQDWLNGKEYKCKVSNKALPAPIEKTISKAKG |
| | | | | QPREPQVYTLPPSREEMTKNQVSLTCLVKGFY |
| | | | | PSDIAVEWESNGQPENNYKTTPPVLDSDGSFFL |
| | | | | YSKLTVDKSRWQQGNVFSCSVMHEALHNHYT |
| | | | | QKSLSLSPGKGGGGSGGGGSGGGGSGGGGSG |
| | | | | GGGSGGGGSDKTHTCPPCPAPELLGGPSVFLFP |
| | | | | PKPKDTLMISRTPEVTCVVVDVSHEDPEVKFN |
| | | | | WYVDGVEVHNAKTKPCEEQYGSTYRCVSVLT |
| | | | | VLHQDWLNGKEYKCKVSNKALPAPIEKTISKA |
| | | | | KGQPREPQVYTLPPSREEMTKNQVSLTCLVKG |
| | | | | FYPSDIAVEWESNGQPENNYKTTPPVLDSDGSF |
| | | | | FLYSKLTVDKSRWQQGNVFSCSVMHEALHNH |
| | | | | YTQKSLSLSPGKGGGGSGGGGSGGGGSEVQLV |
| | | | | ESGGGLVQPGGSLKLSCAASGFTFNKYAMNW |
| | | | | VRQAPGKCLEWVARIRSKYNNYATYYADSVK |
| | | | | DRFTISRDDSKNTAYLQMNNLKTEDTAVYYCV |
| | | | | RHGNFGNSYISYWAYWGQGTLVTVSSGGGGS |
| | | | | GGGGSGGGGSQTVVTQEPSLTVSPGGTVTLTC |
| | | | | GSSTGAVTSGNYPNWVQQKPGQAPRGLIGGTK |
| | | | | FLAPGTPARFSGSLLGGKAALTLSGVQPEDEAE |
| | | | | YYCVLWYSNRWVFGCGTKLTVLGGGGSQVTL |
| | | | | KESGPTLVKPTETLTLTCTVSGFSFRNARMGVS |
| | | | | WIRQPPGKCLEWLAHIFSNDEKSYSTSLKSRLTI |
| | | | | SKDTSKSQVVLTMTNMDPVDTATYFCARVPE |
| | | | | YSSGWYRFDYWGQGTLVTVSSGGGGSGGGGS |
| | | | | GGGGSDIQMTQSPSSLSASVGDRVTISCRASQSI |
| | | | | RSYLNWYQQKPGKAPKLLIYATSSLQGGVPSR |
| | | | | FSGSGSGTDFTLTISSLQPEDFATYYCQQSYSTP |
| | | | | FTFGCGTKVEIK |
| 404. | CL1 9-G4 CC x | artificial | aa | QVQLVQSGAEVKKPGASVKVSCKASGYTFTD |
| | FL 4-E9 CC xscFc | | | YYMHWVRQAPGQCLEWMGWINPNSGGPNYA |
| | x I2Ccc x I2Ccc - | | | QKFQGRVTMTRDTSISTAHMELSRLRSDDTAV |
| | Full Sequence | | | YYCAREKHAVAGIGFDYWGQGTLVTVSSGGG |
| | | | | GSGGGGSGGGGSDIQMTQSPSSVSASVGDRVTI |
| | | | | TCQASQDISNYLNWYQQKPGKAPKLLIYAASS |
| | | | | LESGVPSRFSGSGSGTDFTLTISSLQPEDFATYY |
| | | | | CQQANSFPLTFGCGTKVDIKSGGGGSQVTLKE |
| | | | | SGPTLVKPTETLTLTCTVSGFSFRNARMGVSWI |
| | | | | RQPPGKCLEWLAHIFSNDEKSYSTSLKSRLTISK |
| | | | | DTSKSQVVLTMTNMDPVDTATYFCARVPEYSS |
| | | | | GWYRFDYWGQGTLVTVSSGGGGSGGGGSGG |
| | | | | GGSDIQMTQSPSSLSASVGDRVTISCRASQSIRS |
| | | | | YLNWYQQKPGKAPKLLIYATSSLQGGVPSRFS |
| | | | | GSGSGTDFTLTISSLQPEDFATYYCQQSYSTPFT |
| | | | | FGCGTKVEIKSGGGGDKTHTCPPCPAPELLGGP |
| | | | | SVFLFPPKPKDTLMISRTPEVTCVVVDVSHEDP |
| | | | | EVKFNWYVDGVEVHNAKTKPCEEQYGSTYRC |
| | | | | VSVLTVLHQDWLNGKEYKCKVSNKALPAPIEK |
| | | | | TISKAKGQPREPQVYTLPPSREEMTKNQVSLTC |
| | | | | LVKGFYPSDIAVEWESNGQPENNYKTTPPVLD |
| | | | | SDGSFFLYSKLTVDKSRWQQGNVFSCSVMHEA |
| | | | | LHNHYTQKSLSLSPGKGGGGSGGGGSGGGGS |
| | | | | GGGGSGGGGSGGGGSDKTHTCPPCPAPELLGG |
| | | | | PSVFLFPPKPKDTLMISRTPEVTCVVVDVSHED |
| | | | | PEVKFNWYVDGVEVHNAKTKPCEEQYGSTYR |
| | | | | CVSVLTVLHQDWLNGKEYKCKVSNKALPAPIE |
| | | | | KTISKAKGQPREPQVYTLPPSREEMTKNQVSLT |
| | | | | CLVKGFYPSDIAVEWESNGQPENNYKTTPPVL |
| | | | | DSDGSFFLYSKLTVDKSRWQQGNVFSCSVMHE |
| | | | | ALHNHYTQKSLSLSPGKGGGGEVQLVESGGGL |
| | | | | VQPGGSLKLSCAASGFTFNKYAMNWVRQAPG |
| | | | | KCLEWVARIRSKYNNYATYYADSVKDRFTISR |
| | | | | DDSKNTAYLQMNNLKTEDTAVYYCVRHGNFG |
| | | | | NSYISYWAYWGQGTLVTVSSGGGGSGGGGSG |
| | | | | GGGSQTVVTQEPSLTVSPGGTVTLTCGSSTGA |
| | | | | VTSGNYPNWVQQKPGQAPRGLIGGTKFLAPGT |
| | | | | PARFSGSLLGGKAALTLSGVQPEDEAEYYCVL |
| | | | | WYSNRWVFGCGTKLTVLGGGGSGGGGSGGG |
| | | | | GSEVQLVESGGGLVQPGGSLKLSCAASGFTFN |
| | | | | KYAMNWVRQAPGKCLEWVARIRSKYNNYAT |
| | | | | YYADSVKDRFTISRDDSKNTAYLQMNNLKTED |
| | | | | TAVYYCVRHGNFGNSYISYWAYWGQGTLVTV |
| | | | | SSGGGGSGGGGSGGGGSQTVVTQEPSLTVSPG |
| | | | | GTVTLTCGSSTGAVTSGNYPNWVQQKPGQAP |
| | | | | RGLIGGTKFLAPGTPARFSGSLLGGKAALTLSG |
| | | | | VQPEDEAEYYCVLWYSNRWVFGCGTKLTVL |
| 405. | CL1 9-G4 CC x | artificial | aa | QVQLVQSGAEVKKPGASVKVSCKASGYTFTD |
| | FL 4-E9 CCx | | | YYMHWVRQAPGQCLEWMGWINPNSGGPNYA |
| | I2Ccc x scFc x | | | QKFQGRVTMTRDTSISTAHMELSRLRSDDTAV |
| | I2Ccc - Full | | | YYCAREKHAVAGIGFDYWGQGTLVTVSSGGG |
| | Sequence | | | GSGGGGSGGGGSDIQMTQSPSSVSASVGDRVTI |
| | | | | TCQASQDISNYLNWYQQKPGKAPKLLIYAASS |
| | | | | LESGVPSRFSGSGSGTDFTLTISSLQPEDFATYY |
| | | | | CQQANSFPLTFGCGTKVDIKSGGGGSQVTLKE |
| | | | | SGPTLVKPTETLTLTCTVSGFSFRNARMGVSWI |
| | | | | RQPPGKCLEWLAHIFSNDEKSYSTSLKSRLTISK |
| | | | | DTSKSQVVLTMTNMDPVDTATYFCARVPEYSS |
| | | | | GWYRFDYWGQGTLVTVSSGGGGSGGGGSGG |
| | | | | GGSDIQMTQSPSSLSASVGDRVTISCRASQSIRS |
| | | | | YLNWYQQKPGKAPKLLIYATSSLQGGVPSRFS |
| | | | | GSGSGTDFTLTISSLQPEDFATYYCQQSYSTPFT |
| | | | | FGCGTKVEIKSGGGGSEVQLVESGGGLVQPGG |
| | | | | SLKLSCAASGFTFNKYAMNWVRQAPGKCLEW |
| | | | | VARIRSKYNNYATYYADSVKDRFTISRDDSKN |
| | | | | TAYLQMNNLKTEDTAVYYCVRHGNFGNSYIS |
| | | | | YWAYWGQGTLVTVSSGGGGSGGGGSGGGGS |
| | | | | QTVVTQEPSLTVSPGGTVTLTCGSSTGAVTSGN |
| | | | | YPNWVQQKPGQAPRGLIGGTKFLAPGTPARFS |
| | | | | GSLLGGKAALTLSGVQPEDEAEYYCVLWYSN |
| | | | | RWVFGCGTKLTVLGGGGDKTHTCPPCPAPELL |
| | | | | GGPSVFLFPPKPKDTLMISRTPEVTCVVVDVSH |
| | | | | EDPEVKFNWYVDGVEVHNAKTKPCEEQYGST |
| | | | | YRCVSVLTVLHQDWLNGKEYKCKVSNKALPA |
| | | | | PIEKTISKAKGQPREPQVYTLPPSREEMTKNQV |
| | | | | SLTCLVKGFYPSDIAVEWESNGQPENNYKTTPP |
| | | | | VLDSDGSFFLYSKLTVDKSRWQQGNVFSCSVM |
| | | | | HEALHNHYTQKSLSLSPGKGGGGSGGGGSGG |
| | | | | GGSGGGGSGGGGSGGGGSDKTHTCPPCPAPEL |
| | | | | LGGPSVFLFPPKPKDTLMISRTPEVTCVVVDVS |
| | | | | HEDPEVKFNWYVDGVEVHNAKTKPCEEQYGS |
| | | | | TYRCVSVLTVLHQDWLNGKEYKCKVSNKALP |
| | | | | APIEKTISKAKGQPREPQVYTLPPSREEMTKNQ |
| | | | | VSLTCLVKGFYPSDIAVEWESNGQPENNYKTT |
| | | | | PPVLDSDGSFFLYSKLTVDKSRWQQGNVFSCS |
| | | | | VMHEALHNHYTQKSLSLSPGKGGGGEVQLVE |
| | | | | SGGGLVQPGGSLKLSCAASGFTFNKYAMNWV |
| | | | | RQAPGKCLEWVARIRSKYNNYATYYADSVKD |
| | | | | RFTISRDDSKNTAYLQMNNLKTEDTAVYYCVR |
| | | | | HGNFGNSYISYWAYWGQGTLVTVSSGGGGSG |
| | | | | GGGSGGGGSQTVVTQEPSLTVSPGGTVTLTCG |
| | | | | SSTGAVTSGNYPNWVQQKPGQAPRGLIGGTKF |
| | | | | LAPGTPARFSGSLLGGKAALTLSGVQPEDEAE |
| | | | | YYCVLWYSNRWVFGCGTKLTVL |
| 406. | CL1 9-G4 CC x | artificial | Aa | QVQLVQSGAEVKKPGASVKVSCKASGYTFTD |
| | FL 4-E9 CCx | | | YYMHWVRQAPGQCLEWMGWINPNSGGPNYA |
| | I2Ccc x I2Ccc | | | QKFQGRVTMTRDTSISTAHMELSRLRSDDTAV |
| | xscFc - Full | | | YYCAREKHAVAGIGFDYWGQGTLVTVSSGGG |
| | Sequence | | | GSGGGGSGGGGSDIQMTQSPSSVSASVGDRVTI |
| | | | | TCQASQDISNYLNWYQQKPGKAPKLLIYAASS |
| | | | | LESGVPSRFSGSGSGTDFTLTISSLQPEDFATYY |
| | | | | CQQANSFPLTFGCGTKVDIKSGGGGSQVTLKE |
| | | | | SGPTLVKPTETLTLTCTVSGFSFRNARMGVSWI |
| | | | | RQPPGKCLEWLAHIFSNDEKSYSTSLKSRLTISK |
| | | | | DTSKSQVVLTMTNMDPVDTATYFCARVPEYSS |
| | | | | GWYRFDYWGQGTLVTVSSGGGGSGGGGSGG |
| | | | | GGSDIQMTQSPSSLSASVGDRVTISCRASQSIRS |
| | | | | YLNWYQQKPGKAPKLLIYATSSLQGGVPSRES |
| | | | | GSGSGTDFTLTISSLQPEDFATYYCQQSYSTPFT |
| | | | | FGCGTKVEIKSGGGGSEVQLVESGGGLVQPGG |
| | | | | SLKLSCAASGFTFNKYAMNWVRQAPGKCLEW |
| | | | | VARIRSKYNNYATYYADSVKDRFTISRDDSKN |
| | | | | TAYLQMNNLKTEDTAVYYCVRHGNFGNSYIS |
| | | | | YWAYWGQGTLVTVSSGGGGSGGGGSGGGGS |
| | | | | QTVVTQEPSLTVSPGGTVTLTCGSSTGAVTSGN |
| | | | | YPNWVQQKPGQAPRGLIGGTKFLAPGTPARFS |
| | | | | GSLLGGKAALTLSGVQPEDEAEYYCVLWYSN |
| | | | | RWVFGCGTKLTVLGGGGSGGGGSGGGGSEVQ |
| | | | | LVESGGGLVQPGGSLKLSCAASGFTFNKYAMN |
| | | | | WVRQAPGKCLEWVARIRSKYNNYATYYADSV |
| | | | | KDRFTISRDDSKNTAYLQMNNLKTEDTAVYYC |
| | | | | VRHGNFGNSYISYWAYWGQGTLVTVSSGGGG |
| | | | | SGGGGSGGGGSQTVVTQEPSLTVSPGGTVTLT |
| | | | | CGSSTGAVTSGNYPNWVQQKPGQAPRGLIGGT |
| | | | | KFLAPGTPARFSGSLLGGKAALTLSGVQPEDEA |
| | | | | EYYCVLWYSNRWVFGCGTKLTVLGGGGDKT |
| | | | | HTCPPCPAPELLGGPSVFLFPPKPKDTLMISRTP |
| | | | | EVTCVVVDVSHEDPEVKFNWYVDGVEVHNA |
| | | | | KTKPCEEQYGSTYRCVSVLTVLHQDWLNGKE |
| | | | | YKCKVSNKALPAPIEKTISKAKGQPREPQVYTL |
| | | | | PPSREEMTKNQVSLTCLVKGFYPSDIAVEWES |
| | | | | NGQPENNYKTTPPVLDSDGSFFLYSKLTVDKS |
| | | | | RWQQGNVFSCSVMHEALHNHYTQKSLSLSPG |
| | | | | KGGGGSGGGGSGGGGSGGGGSGGGGSGGGGS |
| | | | | DKTHTCPPCPAPELLGGPSVFLFPPKPKDTLMIS |
| | | | | RTPEVTCVVVDVSHEDPEVKFNWYVDGVEVH |
| | | | | NAKTKPCEEQYGSTYRCVSVLTVLHQDWLNG |
| | | | | KEYKCKVSNKALPAPIEKTISKAKGQPREPQVY |
| | | | | TLPPSREEMTKNQVSLTCLVKGFYPSDIAVEWE |
| | | | | SNGQPENNYKTTPPVLDSDGSFFLYSKLTVDKS |
| | | | | RWQQGNVFSCSVMHEALHNHYTQKSLSLSPG |
| | | | | K |
| 407. | CL1 9-G4 CC x | artificial | Aa | QVQLVQSGAEVKKPGASVKVSCKASGYTFTD |
| | I2Ccc xG4 xscFc | | | YYMHWVRQAPGQCLEWMGWINPNSGGPNYA |
| | xG4 xFL 4-E9 CC | | | QKFQGRVTMTRDTSISTAHMELSRLRSDDTAV |
| | x I2Ccc - Full | | | YYCAREKHAVAGIGFDYWGQGTLVTVSSGGG |
| | Sequence | | | GSGGGGSGGGGSDIQMTQSPSSVSASVGDRVTI |
| | | | | TCQASQDISNYLNWYQQKPGKAPKLLIYAASS |
| | | | | LESGVPSRFSGSGSGTDFTLTISSLQPEDFATYY |
| | | | | CQQANSFPLTFGCGTKVDIKSGGGGSEVQLVE |
| | | | | SGGGLVQPGGSLKLSCAASGFTFNKYAMNWV |
| | | | | RQAPGKCLEWVARIRSKYNNYATYYADSVKD |
| | | | | RFTISRDDSKNTAYLQMNNLKTEDTAVYYCVR |
| | | | | HGNFGNSYISYWAYWGQGTLVTVSSGGGGSG |
| | | | | GGGSGGGGSQTVVTQEPSLTVSPGGTVTLTCG |
| | | | | SSTGAVTSGNYPNWVQQKPGQAPRGLIGGTKF |
| | | | | LAPGTPARFSGSLLGGKAALTLSGVQPEDEAE |
| | | | | YYCVLWYSNRWVFGCGTKLTVLGGGGDKTH |
| | | | | TCPPCPAPELLGGPSVFLFPPKPKDTLMISRTPE |
| | | | | VTCVVVDVSHEDPEVKFNWYVDGVEVHNAK |
| | | | | TKPCEEQYGSTYRCVSVLTVLHQDWLNGKEY |
| | | | | KCKVSNKALPAPIEKTISKAKGQPREPQVYTLP |
| | | | | PSREEMTKNQVSLTCLVKGFYPSDIAVEWESN |
| | | | | GQPENNYKTTPPVLDSDGSFFLYSKLTVDKSR |
| | | | | WQQGNVFSCSVMHEALHNHYTQKSLSLSPGK |
| | | | | GGGGSGGGGSGGGGSGGGGSGGGGSGGGGSD |
| | | | | KTHTCPPCPAPELLGGPSVFLFPPKPKDTLMISR |
| | | | | TPEVTCVVVDVSHEDPEVKFNWYVDGVEVHN |
| | | | | AKTKPCEEQYGSTYRCVSVLTVLHQDWLNGK |
| | | | | EYKCKVSNKALPAPIEKTISKAKGQPREPQVYT |
| | | | | LPPSREEMTKNQVSLTCLVKGFYPSDIAVEWES |
| | | | | NGQPENNYKTTPPVLDSDGSFFLYSKLTVDKS |
| | | | | RWQQGNVFSCSVMHEALHNHYTQKSLSLSPG |
| | | | | KGGGGQVTLKESGPTLVKPTETLTLTCTVSGFS |
| | | | | FRNARMGVSWIRQPPGKCLEWLAHIFSNDEKS |
| | | | | YSTSLKSRLTISKDTSKSQVVLTMTNMDPVDT |
| | | | | ATYFCARVPEYSSGWYRFDYWGQGTLVTVSS |
| | | | | GGGGSGGGGSGGGGSDIQMTQSPSSLSASVGD |
| | | | | RVTISCRASQSIRSYLNWYQQKPGKAPKLLIYA |
| | | | | TSSLQGGVPSRFSGSGSGTDFTLTISSLQPEDFA |
| | | | | TYYCQQSYSTPFTFGCGTKVEIKSGGGGSEVQL |
| | | | | VESGGGLVQPGGSLKLSCAASGFTFNKYAMN |
| | | | | WVRQAPGKCLEWVARIRSKYNNYATYYADSV |
| | | | | KDRFTISRDDSKNTAYLQMNNLKTEDTAVYYC |
| | | | | VRHGNFGNSYISYWAYWGQGTLVTVSSGGGG |
| | | | | SGGGGSGGGGSQTVVTQEPSLTVSPGGTVTLT |
| | | | | CGSSTGAVTSGNYPNWVQQKPGQAPRGLIGGT |
| | | | | KFLAPGTPARFSGSLLGGKAALTLSGVQPEDEA |
| | | | | EYYCVLWYSNRWVFGCGTKLTVL |
| 408. | CL1 9-G4 CC | artificial | Aa | QVQLVQSGAEVKKPGASVKVSCKASGYTFTD |
| | xscFc x FL 4-E9 | | | YYMHWVRQAPGQCLEWMGWINPNSGGPNYA |
| | CC x I2Ccc x | | | QKFQGRVTMTRDTSISTAHMELSRLRSDDTAV |
| | I2Ccc - Full | | | YYCAREKHAVAGIGFDYWGQGTLVTVSSGGG |
| | Sequence | | | GSGGGGSGGGGSDIQMTQSPSSVSASVGDRVTI |
| | | | | TCQASQDISNYLNWYQQKPGKAPKLLIYAASS |
| | | | | LESGVPSRFSGSGSGTDFTLTISSLQPEDFATYY |
| | | | | CQQANSFPLTFGCGTKVDIKGGGGDKTHTCPP |
| | | | | CPAPELLGGPSVFLFPPKPKDTLMISRTPEVTCV |
| | | | | VVDVSHEDPEVKFNWYVDGVEVHNAKTKPCE |
| | | | | EQYGSTYRCVSVLTVLHQDWLNGKEYKCKVS |
| | | | | NKALPAPIEKTISKAKGQPREPQVYTLPPSREE |
| | | | | MTKNQVSLTCLVKGFYPSDIAVEWESNGQPEN |
| | | | | NYKTTPPVLDSDGSFFLYSKLTVDKSRWQQGN |
| | | | | VFSCSVMHEALHNHYTQKSLSLSPGKGGGGSG |
| | | | | GGGSGGGGSGGGGSGGGGSGGGGSDKTHTCP |
| | | | | PCPAPELLGGPSVFLFPPKPKDTLMISRTPEVTC |
| | | | | VVVDVSHEDPEVKFNWYVDGVEVHNAKTKPC |
| | | | | EEQYGSTYRCVSVLTVLHQDWLNGKEYKCKV |
| | | | | SNKALPAPIEKTISKAKGQPREPQVYTLPPSREE |
| | | | | MTKNQVSLTCLVKGFYPSDIAVEWESNGQPEN |
| | | | | NYKTTPPVLDSDGSFFLYSKLTVDKSRWQQGN |
| | | | | VFSCSVMHEALHNHYTQKSLSLSPGKGGGGQ |
| | | | | VTLKESGPTLVKPTETLTLTCTVSGFSFRNARM |
| | | | | GVSWIRQPPGKCLEWLAHIFSNDEKSYSTSLKS |
| | | | | RLTISKDTSKSQVVLTMTNMDPVDTATYFCAR |
| | | | | VPEYSSGWYRFDYWGQGTLVTVSSGGGGSGG |
| | | | | GGSGGGGSDIQMTQSPSSLSASVGDRVTISCRA |
| | | | | SQSIRSYLNWYQQKPGKAPKLLIYATSSLQGG |
| | | | | VPSRFSGSGSGTDFTLTISSLQPEDFATYYCQQS |
| | | | | YSTPFTFGCGTKVEIKSGGGGSEVQLVESGGGL |
| | | | | VQPGGSLKLSCAASGFTFNKYAMNWVRQAPG |
| | | | | KCLEWVARIRSKYNNYATYYADSVKDRFTISR |
| | | | | DDSKNTAYLQMNNLKTEDTAVYYCVRHGNFG |
| | | | | NSYISYWAYWGQGTLVTVSSGGGGSGGGGSG |
| | | | | GGGSQTVVTQEPSLTVSPGGTVTLTCGSSTGA |
| | | | | VTSGNYPNWVQQKPGQAPRGLIGGTKFLAPGT |
| | | | | PARFSGSLLGGKAALTLSGVQPEDEAEYYCVL |
| | | | | WYSNRWVFGCGTKLTVLGGGGSGGGGSGGG |
| | | | | GSEVQLVESGGGLVQPGGSLKLSCAASGFTFN |
| | | | | KYAMNWVRQAPGKCLEWVARIRSKYNNYAT |
| | | | | YYADSVKDRFTISRDDSKNTAYLQMNNLKTED |
| | | | | TAVYYCVRHGNFGNSYISYWAYWGQGTLVTV |
| | | | | SSGGGGSGGGGSGGGGSQTVVTQEPSLTVSPG |
| | | | | GTVTLTCGSSTGAVTSGNYPNWVQQKPGQAP |
| | | | | RGLIGGTKFLAPGTPARFSGSLLGGKAALTLSG |
| | | | | VQPEDEAEYYCVLWYSNRWVFGCGTKLTVL |
| 409. | CL1_9- | artificial | Aa | QVQLVQSGAEVKKPGASVKVSCKASGYTFTD |
| | G4_CC_x(EAAA | | | YYMHWVRQAPGQCLEWMGWINPNSGGPNYA |
| | K)10_x | | | QKFQGRVTMTRDTSISTAHMELSRLRSDDTAV |
| | I2Ccc_xscFc_xFL | | | YYCAREKHAVAGIGFDYWGQGTLVTVSSGGG |
| | _4- | | | GSGGGGSGGGGSDIQMTQSPSSVSASVGDRVTI |
| | E9_CC_x(EAAA | | | TCQASQDISNYLNWYQQKPGKAPKLLIYAASS |
| | K)10_x I2Ccc - | | | LESGVPSRFSGSGSGTDFTLTISSLQPEDFATYY |
| | Full Sequence | | | CQQANSFPLTFGCGTKVDIKSGEAAAKEAAAK |
| | | | | EAAAKEAAAKEAAAKEAAAKEAAAKEAAAK |
| | | | | EAAAKEAAAKEVQLVESGGGLVQPGGSLKLS |
| | | | | CAASGFTFNKYAMNWVRQAPGKCLEWVARIR |
| | | | | SKYNNYATYYADSVKDRFTISRDDSKNTAYLQ |
| | | | | MNNLKTEDTAVYYCVRHGNFGNSYISYWAY |
| | | | | WGQGTLVTVSSGGGGSGGGGSGGGGSQTVVT |
| | | | | QEPSLTVSPGGTVTLTCGSSTGAVTSGNYPNW |
| | | | | VQQKPGQAPRGLIGGTKFLAPGTPARFSGSLLG |
| | | | | GKAALTLSGVQPEDEAEYYCVLWYSNRWVFG |
| | | | | CGTKLTVLGGGGDKTHTCPPCPAPELLGGPSV |
| | | | | FLFPPKPKDTLMISRTPEVTCVVVDVSHEDPEV |
| | | | | KFNWYVDGVEVHNAKTKPCEEQYGSTYRCVS |
| | | | | VLTVLHQDWLNGKEYKCKVSNKALPAPIEKTI |
| | | | | SKAKGQPREPQVYTLPPSREEMTKNQVSLTCL |
| | | | | VKGFYPSDIAVEWESNGQPENNYKTTPPVLDS |
| | | | | DGSFFLYSKLTVDKSRWQQGNVFSCSVMHEA |
| | | | | LHNHYTQKSLSLSPGKGGGGSGGGGSGGGGS |
| | | | | GGGGSGGGGSGGGGSDKTHTCPPCPAPELLGG |
| | | | | PSVFLFPPKPKDTLMISRTPEVTCVVVDVSHED |
| | | | | PEVKFNWYVDGVEVHNAKTKPCEEQYGSTYR |
| | | | | CVSVLTVLHQDWLNGKEYKCKVSNKALPAPIE |
| | | | | KTISKAKGQPREPQVYTLPPSREEMTKNQVSLT |
| | | | | CLVKGFYPSDIAVEWESNGQPENNYKTTPPVL |
| | | | | DSDGSFFLYSKLTVDKSRWQQGNVFSCSVMHE |
| | | | | ALHNHYTQKSLSLSPGKGGGGQVTLKESGPTL |
| | | | | VKPTETLTLTCTVSGFSFRNARMGVSWIRQPPG |
| | | | | KCLEWLAHIFSNDEKSYSTSLKSRLTISKDTSKS |
| | | | | QVVLTMTNMDPVDTATYFCARVPEYSSGWYR |
| | | | | FDYWGQGTLVTVSSGGGGSGGGGSGGGGSDI |
| | | | | QMTQSPSSLSASVGDRVTISCRASQSIRSYLNW |
| | | | | YQQKPGKAPKLLIYATSSLQGGVPSRFSGSGSG |
| | | | | TDFTLTISSLQPEDFATYYCQQSYSTPFTFGCGT |
| | | | | KVEIKSGEAAAKEAAAKEAAAKEAAAKEAAA |
| | | | | KEAAAKEAAAKEAAAKEAAAKEAAAKEVQL |
| | | | | VESGGGLVQPGGSLKLSCAASGFTFNKYAMN |
| | | | | WVRQAPGKCLEWVARIRSKYNNYATYYADSV |
| | | | | KDRFTISRDDSKNTAYLQMNNLKTEDTAVYYC |
| | | | | VRHGNFGNSYISYWAYWGQGTLVTVSSGGGG |
| | | | | SGGGGSGGGGSQTVVTQEPSLTVSPGGTVTLT |
| | | | | CGSSTGAVTSGNYPNWVQQKPGQAPRGLIGGT |
| | | | | KFLAPGTPARFSGSLLGGKAALTLSGVQPEDEA |
| | | | | EYYCVLWYSNRWVFGCGTKLTVL |
| 410. | CL1_9- | artificial | Aa | QVQLVQSGAEVKKPGASVKVSCKASGYTFTD |
| | G4_CC_x(G4S)3_ | | | YYMHWVRQAPGQCLEWMGWINPNSGGPNYA |
| | x | | | QKFQGRVTMTRDTSISTAHMELSRLRSDDTAV |
| | I2Ccc_xscFc_xFL | | | YYCAREKHAVAGIGFDYWGQGTLVTVSSGGG |
| | _4- | | | GSGGGGSGGGGSDIQMTQSPSSVSASVGDRVTI |
| | E9_CC_x(G4S)3_ | | | TCQASQDISNYLNWYQQKPGKAPKLLIYAASS |
| | x _2Ccc - Full | | | LESGVPSRFSGSGSGTDFTLTISSLQPEDFATYY |
| | Sequence | | | CQQANSFPLTFGCGTKVDIKSGGGGSGGGGSG |
| | | | | GGGSEVQLVESGGGLVQPGGSLKLSCAASGFT |
| | | | | FNKYAMNWVRQAPGKCLEWVARIRSKYNNY |
| | | | | ATYYADSVKDRFTISRDDSKNTAYLQMNNLKT |
| | | | | EDTAVYYCVRHGNFGNSYISYWAYWGQGTLV |
| | | | | TVSSGGGGSGGGGSGGGGSQTVVTQEPSLTVS |
| | | | | PGGTVTLTCGSSTGAVTSGNYPNWVQQKPGQ |
| | | | | APRGLIGGTKFLAPGTPARFSGSLLGGKAALTL |
| | | | | SGVQPEDEAEYYCVLWYSNRWVFGCGTKLTV |
| | | | | LGGGGDKTHTCPPCPAPELLGGPSVFLFPPKPK |
| | | | | DTLMISRTPEVTCVVVDVSHEDPEVKFNWYVD |
| | | | | GVEVHNAKTKPCEEQYGSTYRCVSVLTVLHQ |
| | | | | DWLNGKEYKCKVSNKALPAPIEKTISKAKGQP |
| | | | | REPQVYTLPPSREEMTKNQVSLTCLVKGFYPS |
| | | | | DIAVEWESNGQPENNYKTTPPVLDSDGSFFLYS |
| | | | | KLTVDKSRWQQGNVFSCSVMHEALHNHYTQK |
| | | | | SLSLSPGKGGGGSGGGGSGGGGSGGGGSGGG |
| | | | | GSGGGGSDKTHTCPPCPAPELLGGPSVFLFPPK |
| | | | | PKDTLMISRTPEVTCVVVDVSHEDPEVKFNWY |
| | | | | VDGVEVHNAKTKPCEEQYGSTYRCVSVLTVL |
| | | | | HQDWLNGKEYKCKVSNKALPAPIEKTISKAKG |
| | | | | QPREPQVYTLPPSREEMTKNQVSLTCLVKGFY |
| | | | | PSDIAVEWESNGQPENNYKTTPPVLDSDGSFFL |
| | | | | YSKLTVDKSRWQQGNVFSCSVMHEALHNHYT |
| | | | | QKSLSLSPGKGGGGQVTLKESGPTLVKPTETLT |
| | | | | LTCTVSGFSFRNARMGVSWIRQPPGKCLEWLA |
| | | | | HIFSNDEKSYSTSLKSRLTISKDTSKSQVVLTMT |
| | | | | NMDPVDTATYFCARVPEYSSGWYRFDYWGQ |
| | | | | GTLVTVSSGGGGSGGGGSGGGGSDIQMTQSPS |
| | | | | SLSASVGDRVTISCRASQSIRSYLNWYQQKPGK |
| | | | | APKLLIYATSSLQGGVPSRFSGSGSGTDFTLTIS |
| | | | | SLQPEDFATYYCQQSYSTPFTFGCGTKVEIKSG |
| | | | | GGGSGGGGSGGGGSEVQLVESGGGLVQPGGS |
| | | | | LKLSCAASGFTFNKYAMNWVRQAPGKCLEWV |
| | | | | ARIRSKYNNYATYYADSVKDRFTISRDDSKNT |
| | | | | AYLQMNNLKTEDTAVYYCVRHGNFGNSYISY |
| | | | | WAYWGQGTLVTVSSGGGGSGGGGSGGGGSQ |
| | | | | TVVTQEPSLTVSPGGTVTLTCGSSTGAVTSGNY |
| | | | | PNWVQQKPGQAPRGLIGGTKFLAPGTPARFSG |
| | | | | SLLGGKAALTLSGVQPEDEAEYYCVLWYSNR |
| | | | | WVFGCGTKLTVL |
| 411. | CH3-G8A_6- | artificial | Aa | EVQLLESGGGLVQPGGSLRLSCAASGFSFSSYPI |
| | B12x I2Cx | | | NWVRQAPGKGLEWVGVIWTGGGTNYASSVK |
| | scFc_(G4S)6 - | | | GRFTISRDNSKNTVYLQMNSLRAEDTAVYYCA |
| | Full Sequence | | | KSRGVYDFDGRGAMDYWGQGTLVTVSSGGG |
| | | | | GSGGGGSGGGGSDIVMTQSPDSLAVSLGERATI |
| | | | | NCKSSQSLLYSSNQKNYFAWYQQKPGQPPKLL |
| | | | | IYWASTRESGVPDRFSGSGSGTDFTLTISSLQAE |
| | | | | DVAVYYCQQYYSYPYTFGQGTKLEIKSGGGGS |
| | | | | EVQLVESGGGLVQPGGSLKLSCAASGFTFNKY |
| | | | | AMNWVRQAPGKGLEWVARIRSKYNNYATYY |
| | | | | ADSVKDRFTISRDDSKNTAYLQMNNLKTEDTA |
| | | | | VYYCVRHGNFGNSYISYWAYWGQGTLVTVSS |
| | | | | GGGGSGGGGSGGGGSQTVVTQEPSLTVSPGGT |
| | | | | VTLTCGSSTGAVTSGNYPNWVQQKPGQAPRG |
| | | | | LIGGTKFLAPGTPARFSGSLLGGKAALTLSGVQ |
| | | | | PEDEAEYYCVLWYSNRWVFGGGTKLTVLGGG |
| | | | | GDKTHTCPPCPAPELLGGPSVFLFPPKPKDTLM |
| | | | | ISRTPEVTCVVVDVSHEDPEVKFNWYVDGVEV |
| | | | | HNAKTKPCEEQYGSTYRCVSVLTVLHQDWLN |
| | | | | GKEYKCKVSNKALPAPIEKTISKAKGQPREPQV |
| | | | | YTLPPSREEMTKNQVSLTCLVKGFYPSDIAVE |
| | | | | WESNGQPENNYKTTPPVLDSDGSFFLYSKLTV |
| | | | | DKSRWQQGNVFSCSVMHEALHNHYTQKSLSL |
| | | | | SPGKGGGGSGGGGSGGGGSGGGGSGGGGSGG |
| | | | | GGSDKTHTCPPCPAPELLGGPSVFLFPPKPKDT |
| | | | | LMISRTPEVTCVVVDVSHEDPEVKFNWYVDG |
| | | | | VEVHNAKTKPCEEQYGSTYRCVSVLTVLHQD |
| | | | | WLNGKEYKCKVSNKALPAPIEKTISKAKGQPR |
| | | | | EPQVYTLPPSREEMTKNQVSLTCLVKGFYPSDI |
| | | | | AVEWESNGQPENNYKTTPPVLDSDGSFFLYSK |
| | | | | LTVDKSRWQQGNVFSCSVMHEALHNHYTQKS |
| | | | | LSLSPGK |
| 412. | CL1 9-G4 CC x | artificial | Aa | QVQLVQSGAEVKKPGASVKVSCKASGYTFTD |
| | I2C x scFc - Full | | | YYMHWVRQAPGQCLEWMGWINPNSGGPNYA |
| | Sequence | | | QKFQGRVTMTRDTSISTAHMELSRLRSDDTAV |
| | | | | YYCAREKHAVAGIGFDYWGQGTLVTVSSGGG |
| | | | | GSGGGGSGGGGSDIQMTQSPSSVSASVGDRVTI |
| | | | | TCQASQDISNYLNWYQQKPGKAPKLLIYAASS |
| | | | | LESGVPSRFSGSGSGTDFTLTISSLQPEDFATYY |
| | | | | CQQANSFPLTFGCGTKVDIKSGGGGSEVQLVE |
| | | | | SGGGLVQPGGSLKLSCAASGFTFNKYAMNWV |
| | | | | RQAPGKGLEWVARIRSKYNNYATYYADSVKD |
| | | | | RFTISRDDSKNTAYLQMNNLKTEDTAVYYCVR |
| | | | | HGNFGNSYISYWAYWGQGTLVTVSSGGGGSG |
| | | | | GGGSGGGGSQTVVTQEPSLTVSPGGTVTLTCG |
| | | | | SSTGAVTSGNYPNWVQQKPGQAPRGLIGGTKF |
| | | | | LAPGTPARFSGSLLGGKAALTLSGVQPEDEAE |
| | | | | YYCVLWYSNRWVFGGGTKLTVLGGGGDKTH |
| | | | | TCPPCPAPELLGGPSVFLFPPKPKDTLMISRTPE |
| | | | | VTCVVVDVSHEDPEVKFNWYVDGVEVHNAK |
| | | | | TKPCEEQYGSTYRCVSVLTVLHQDWLNGKEY |
| | | | | KCKVSNKALPAPIEKTISKAKGQPREPQVYTLP |
| | | | | PSREEMTKNQVSLTCLVKGFYPSDIAVEWESN |
| | | | | GQPENNYKTTPPVLDSDGSFFLYSKLTVDKSR |
| | | | | WQQGNVFSCSVMHEALHNHYTQKSLSLSPGK |
| | | | | GGGGSGGGGSGGGGSGGGGSGGGGSGGGGSD |
| | | | | KTHTCPPCPAPELLGGPSVFLFPPKPKDTLMISR |
| | | | | TPEVTCVVVDVSHEDPEVKFNWYVDGVEVHN |
| | | | | AKTKPCEEQYGSTYRCVSVLTVLHQDWLNGK |
| | | | | EYKCKVSNKALPAPIEKTISKAKGQPREPQVYT |
| | | | | LPPSREEMTKNQVSLTCLVKGFYPSDIAVEWES |
| | | | | NGQPENNYKTTPPVLDSDGSFFLYSKLTVDKS |
| | | | | RWQQGNVFSCSVMHEALHNHYTQKSLSLSPG |
| | | | | K |
| 413. | CL1 9-G4 CC x | artificial | Aa | QVQLVQSGAEVKKPGASVKVSCKASGYTFTD |
| | PSMA 76-B10 x | | | YYMHWVRQAPGQCLEWMGWINPNSGGPNYA |
| | I2C x scFc - Full | | | QKFQGRVTMTRDTSISTAHMELSRLRSDDTAV |
| | Sequence | | | YYCAREKHAVAGIGFDYWGQGTLVTVSSGGG |
| | | | | GSGGGGSGGGGSDIQMTQSPSSVSASVGDRVTI |
| | | | | TCQASQDISNYLNWYQQKPGKAPKLLIYAASS |
| | | | | LESGVPSRFSGSGSGTDFTLTISSLQPEDFATYY |
| | | | | CQQANSFPLTFGCGTKVDIKSGGGGSQVQLVE |
| | | | | SGGGLVKPGESLRLSCAASGFTFSDYYMYWVR |
| | | | | QAPGKGLEWVAIISDGGYYTYYSDIIKGRFTISR |
| | | | | DNAKNSLYLQMNSLKAEDTAVYYCARGFPLL |
| | | | | RHGAMDYWGQGTLVTVSSGGGGSGGGGSGG |
| | | | | GGSDIQMTQSPSSLSASVGDRVTITCKASQNVD |
| | | | | TNVAWYQQKPGQAPKSLIYSASYRYSDVPSRF |
| | | | | SGSASGTDFTLTISSVQSEDFATYYCQQYDSYP |
| | | | | YTFGGGTKLEIKSGGGGSEVQLVESGGGLVQP |
| | | | | GGSLKLSCAASGFTFNKYAMNWVRQAPGKGL |
| | | | | EWVARIRSKYNNYATYYADSVKDRFTISRDDS |
| | | | | KNTAYLQMNNLKTEDTAVYYCVRHGNFGNSY |
| | | | | ISYWAYWGQGTLVTVSSGGGGSGGGGSGGGG |
| | | | | SQTVVTQEPSLTVSPGGTVTLTCGSSTGAVTSG |
| | | | | NYPNWVQQKPGQAPRGLIGGTKFLAPGTPARF |
| | | | | SGSLLGGKAALTLSGVQPEDEAEYYCVLWYSN |
| | | | | RWVFGGGTKLTVLGGGGDKTHTCPPCPAPELL |
| | | | | GGPSVFLFPPKPKDTLMISRTPEVTCVVVDVSH |
| | | | | EDPEVKFNWYVDGVEVHNAKTKPCEEQYGST |
| | | | | YRCVSVLTVLHQDWLNGKEYKCKVSNKALPA |
| | | | | PIEKTISKAKGQPREPQVYTLPPSREEMTKNQV |
| | | | | SLTCLVKGFYPSDIAVEWESNGQPENNYKTTPP |
| | | | | VLDSDGSFFLYSKLTVDKSRWQQGNVFSCSVM |
| | | | | HEALHNHYTQKSLSLSPGKGGGGSGGGGSGG |
| | | | | GGSGGGGSGGGGSGGGGSDKTHTCPPCPAPEL |
| | | | | LGGPSVFLFPPKPKDTLMISRTPEVTCVVVDVS |
| | | | | HEDPEVKFNWYVDGVEVHNAKTKPCEEQYGS |
| | | | | TYRCVSVLTVLHQDWLNGKEYKCKVSNKALP |
| | | | | APIEKTISKAKGQPREPQVYTLPPSREEMTKNQ |
| | | | | VSLTCLVKGFYPSDIAVEWESNGQPENNYKTT |
| | | | | PPVLDSDGSFFLYSKLTVDKSRWQQGNVFSCS |
| | | | | VMHEALHNHYTQKSLSLSPGK |
| 414. | EGFRvIII_CC_x | artificial | Aa | QVQLVESGGGVVQSGRSLRLSCAASGFTFRNY |
| | I2C x scFc - Full | | | GMHWVRQAPGKCLEWVAVIWYDGSDKYYAD |
| | Sequence | | | SVRGRFTISRDNSKNTLYLQMNSLRAEDTAVY |
| | | | | YCARDGYDILTGNPRDFDYWGQGTLVTVSSG |
| | | | | GGGSGGGGSGGGGSDTVMTQTPLSSHVTLGQP |
| | | | | ASISCRSSQSLVHSDGNTYLSWLQQRPGQPPRL |
| | | | | LIYRISRRFSGVPDRFSGSGAGTDFTLEISRVEA |
| | | | | EDVGVYYCMQSTHVPRTFGCGTKVEIKSGGG |
| | | | | GSEVQLVESGGGLVQPGGSLKLSCAASGFTFN |
| | | | | KYAMNWVRQAPGKGLEWVARIRSKYNNYAT |
| | | | | YYADSVKDRFTISRDDSKNTAYLQMNNLKTED |
| | | | | TAVYYCVRHGNFGNSYISYWAYWGQGTLVTV |
| | | | | SSGGGGSGGGGSGGGGSQTVVTQEPSLTVSPG |
| | | | | GTVTLTCGSSTGAVTSGNYPNWVQQKPGQAP |
| | | | | RGLIGGTKFLAPGTPARFSGSLLGGKAALTLSG |
| | | | | VQPEDEAEYYCVLWYSNRWVFGGGTKLTVLG |
| | | | | GGGDKTHTCPPCPAPELLGGPSVFLFPPKPKDT |
| | | | | LMISRTPEVTCVVVDVSHEDPEVKFNWYVDG |
| | | | | VEVHNAKTKPCEEQYGSTYRCVSVLTVLHQD |
| | | | | WLNGKEYKCKVSNKALPAPIEKTISKAKGQPR |
| | | | | EPQVYTLPPSREEMTKNQVSLTCLVKGFYPSDI |
| | | | | AVEWESNGQPENNYKTTPPVLDSDGSFFLYSK |
| | | | | LTVDKSRWQQGNVFSCSVMHEALHNHYTQKS |
| | | | | LSLSPGKGGGGSGGGGSGGGGSGGGGSGGGG |
| | | | | SGGGGSDKTHTCPPCPAPELLGGPSVFLFPPKP |
| | | | | KDTLMISRTPEVTCVVVDVSHEDPEVKFNWYV |
| | | | | DGVEVHNAKTKPCEEQYGSTYRCVSVLTVLH |
| | | | | QDWLNGKEYKCKVSNKALPAPIEKTISKAKGQ |
| | | | | PREPQVYTLPPSREEMTKNQVSLTCLVKGFYPS |
| | | | | DIAVEWESNGQPENNYKTTPPVLDSDGSFFLYS |
| | | | | KLTVDKSRWQQGNVFSCSVMHEALHNHYTQK |
| | | | | SLSLSPGK |
| 415. | EpCAM 5-10 x | artificial | aa | ELVMTQSPSSLTVTAGEKVTMSCKSSQSLLNS |
| | I2C x scFc - Full | | | GNQKNYLTWYQQKPGQPPKLLIYWASTRESG |
| | Sequence | | | VPDRFTGSGSGTDFTLTISSVQAEDLAVYYCQN |
| | | | | DYSYPLTFGAGTKLEIKGGGGSGGGGSGGGGS |
| | | | | EVQLLEQSGAELVRPGTSVKISCKASGYAFTNY |
| | | | | WLGWVKQRPGHGLEWIGDIFPGSGNIHYNEKF |
| | | | | KGKATLTADKSSSTAYMQLSSLTFEDSAVYFC |
| | | | | ARLRNWDEPMDYWGQGTTVTVSSGGGGSEV |
| | | | | QLVESGGGLVQPGGSLKLSCAASGFTFNKYAM |
| | | | | NWVRQAPGKGLEWVARIRSKYNNYATYYADS |
| | | | | VKDRFTISRDDSKNTAYLQMNNLKTEDTAVY |
| | | | | YCVRHGNFGNSYISYWAYWGQGTLVTVSSGG |
| | | | | GGSGGGGSGGGGSQTVVTQEPSLTVSPGGTVT |
| | | | | LTCGSSTGAVTSGNYPNWVQQKPGQAPRGLIG |
| | | | | GTKFLAPGTPARFSGSLLGGKAALTLSGVQPED |
| | | | | EAEYYCVLWYSNRWVFGGGTKLTVLGGGGD |
| | | | | KTHTCPPCPAPELLGGPSVFLFPPKPKDTLMISR |
| | | | | TPEVTCVVVDVSHEDPEVKFNWYVDGVEVHN |
| | | | | AKTKPCEEQYGSTYRCVSVLTVLHQDWLNGK |
| | | | | EYKCKVSNKALPAPIEKTISKAKGQPREPQVYT |
| | | | | LPPSREEMTKNQVSLTCLVKGFYPSDIAVEWES |
| | | | | NGQPENNYKTTPPVLDSDGSFFLYSKLTVDKS |
| | | | | RWQQGNVFSCSVMHEALHNHYTQKSLSLSPG |
| | | | | KGGGGSGGGGSGGGGSGGGGSGGGGSGGGGS |
| | | | | DKTHTCPPCPAPELLGGPSVFLFPPKPKDTLMIS |
| | | | | RTPEVTCVVVDVSHEDPEVKFNWYVDGVEVH |
| | | | | NAKTKPCEEQYGSTYRCVSVLTVLHQDWLNG |
| | | | | KEYKCKVSNKALPAPIEKTISKAKGQPREPQVY |
| | | | | TLPPSREEMTKNQVSLTCLVKGFYPSDIAVEWE |
| | | | | SNGQPENNYKTTPPVLDSDGSFFLYSKLTVDKS |
| | | | | RWQQGNVFSCSVMHEALHNHYTQKSLSLSPG |
| | | | | K |
| 416. | FL 4-E9 CC x I2C | artificial | aa | QVTLKESGPTLVKPTETLTLTCTVSGFSFRNAR |
| | x scFc - Full | | | MGVSWIRQPPGKCLEWLAHIFSNDEKSYSTSL |
| | Sequence | | | KSRLTISKDTSKSQVVLTMTNMDPVDTATYFC |
| | | | | ARVPEYSSGWYRFDYWGQGTLVTVSSGGGGS |
| | | | | GGGGSGGGGSDIQMTQSPSSLSASVGDRVTISC |
| | | | | RASQSIRSYLNWYQQKPGKAPKLLIYATSSLQG |
| | | | | GVPSRFSGSGSGTDFTLTISSLQPEDFATYYCQQ |
| | | | | SYSTPFTFGCGTKVEIKSGGGGSEVQLVESGGG |
| | | | | LVQPGGSLKLSCAASGFTFNKYAMNWVRQAP |
| | | | | GKGLEWVARIRSKYNNYATYYADSVKDRFTIS |
| | | | | RDDSKNTAYLQMNNLKTEDTAVYYCVRHGNF |
| | | | | GNSYISYWAYWGQGTLVTVSSGGGGSGGGGS |
| | | | | GGGGSQTVVTQEPSLTVSPGGTVTLTCGSSTG |
| | | | | AVTSGNYPNWVQQKPGQAPRGLIGGTKFLAPG |
| | | | | TPARFSGSLLGGKAALTLSGVQPEDEAEYYCV |
| | | | | LWYSNRWVFGGGTKLTVLGGGGDKTHTCPPC |
| | | | | PAPELLGGPSVFLFPPKPKDTLMISRTPEVTCVV |
| | | | | VDVSHEDPEVKFNWYVDGVEVHNAKTKPCEE |
| | | | | QYGSTYRCVSVLTVLHQDWLNGKEYKCKVSN |
| | | | | KALPAPIEKTISKAKGQPREPQVYTLPPSREEM |
| | | | | TKNQVSLTCLVKGFYPSDIAVEWESNGQPENN |
| | | | | YKTTPPVLDSDGSFFLYSKLTVDKSRWQQGNV |
| | | | | FSCSVMHEALHNHYTQKSLSLSPGKGGGGSGG |
| | | | | GGSGGGGSGGGGSGGGGSGGGGSDKTHTCPP |
| | | | | CPAPELLGGPSVFLFPPKPKDTLMISRTPEVTCV |
| | | | | VVDVSHEDPEVKFNWYVDGVEVHNAKTKPCE |
| | | | | EQYGSTYRCVSVLTVLHQDWLNGKEYKCKVS |
| | | | | NKALPAPIEKTISKAKGQPREPQVYTLPPSREE |
| | | | | MTKNQVSLTCLVKGFYPSDIAVEWESNGQPEN |
| | | | | NYKTTPPVLDSDGSFFLYSKLTVDKSRWQQGN |
| | | | | VFSCSVMHEALHNHYTQKSLSLSPGK |
| 417. | MS 5-F11 x I2C x | artificial | aa | QVQLVESGGGLVKPGGSLRLSCAASGFTFSDY |
| | scFc - Full | | | YMTWIRQAPGKGLEWLSYISSSGSTIYYADSV |
| | Sequence | | | KGRFTISRDNAKNSLFLQMNSLRAEDTAVYYC |
| | | | | ARDRNSHFDYWGQGTLVTVSSGGGGSGGGGS |
| | | | | GGGGSDIQMTQSPSSVSASVGDRVTITCRASQG |
| | | | | INTWLAWYQQKPGKAPKLLIYGASGLQSGVPS |
| | | | | RFSGSGSGTDFTLTISSLQPEDFATYYCQQAKSF |
| | | | | PRTFGQGTKVEIKSGGGGSEVQLVESGGGLVQ |
| | | | | PGGSLKLSCAASGFTFNKYAMNWVRQAPGKG |
| | | | | LEWVARIRSKYNNYATYYADSVKDRFTISRDD |
| | | | | SKNTAYLQMNNLKTEDTAVYYCVRHGNFGNS |
| | | | | YISYWAYWGQGTLVTVSSGGGGSGGGGSGGG |
| | | | | GSQTVVTQEPSLTVSPGGTVTLTCGSSTGAVTS |
| | | | | GNYPNWVQQKPGQAPRGLIGGTKFLAPGTPAR |
| | | | | FSGSLLGGKAALTLSGVQPEDEAEYYCVLWYS |
| | | | | NRWVFGGGTKLTVLGGGGDKTHTCPPCPAPEL |
| | | | | LGGPSVFLFPPKPKDTLMISRTPEVTCVVVDVS |
| | | | | HEDPEVKFNWYVDGVEVHNAKTKPCEEQYGS |
| | | | | TYRCVSVLTVLHQDWLNGKEYKCKVSNKALP |
| | | | | APIEKTISKAKGQPREPQVYTLPPSREEMTKNQ |
| | | | | VSLTCLVKGFYPSDIAVEWESNGQPENNYKTT |
| | | | | PPVLDSDGSFFLYSKLTVDKSRWQQGNVFSCS |
| | | | | VMHEALHNHYTQKSLSLSPGKGGGGSGGGGS |
| | | | | GGGGSGGGGSGGGGSGGGGSDKTHTCPPCPAP |
| | | | | ELLGGPSVFLFPPKPKDTLMISRTPEVTCVVVD |
| | | | | VSHEDPEVKFNWYVDGVEVHNAKTKPCEEQY |
| | | | | GSTYRCVSVLTVLHQDWLNGKEYKCKVSNKA |
| | | | | LPAPIEKTISKAKGQPREPQVYTLPPSREEMTK |
| | | | | NQVSLTCLVKGFYPSDIAVEWESNGQPENNYK |
| | | | | TTPPVLDSDGSFFLYSKLTVDKSRWQQGNVFS |
| | | | | CSVMHEALHNHYTQKSLSLSPGK |
| 418. | MSLN H2 x I2C | artificial | Aa | EVQLLESGGGVVQPGRSLRLSCAASGFTFSSYG |
| | xscFc - Full | | | MGWVRQAPGKGLEWVAVISYDGSNKYYADS |
| | Sequence | | | VKGRFTISRDNSKNTLYLQMNSLRAEDTAVYY |
| | | | | CAREGAHFGSGSYYPLYYYYAMDVWGQGTT |
| | | | | VTVSSGGGGSGGGGSGGGGSELTLTQSPGTLS |
| | | | | LSPGERATLSCRASQSVSSSYLAWYQQKPGQA |
| | | | | PRLLIYGASIRATGIPDRFSGSGSGTDFTLTISRL |
| | | | | EPEDFAVYYCQQYGSSPIFTFGPGTKVEIKSGG |
| | | | | GGSEVQLVESGGGLVQPGGSLKLSCAASGFTF |
| | | | | NKYAMNWVRQAPGKGLEWVARIRSKYNNYA |
| | | | | TYYADSVKDRFTISRDDSKNTAYLQMNNLKTE |
| | | | | DTAVYYCVRHGNFGNSYISYWAYWGQGTLVT |
| | | | | VSSGGGGSGGGGSGGGGSQTVVTQEPSLTVSP |
| | | | | GGTVTLTCGSSTGAVTSGNYPNWVQQKPGQA |
| | | | | PRGLIGGTKFLAPGTPARFSGSLLGGKAALTLS |
| | | | | GVQPEDEAEYYCVLWYSNRWVFGGGTKLTVL |
| | | | | GGGGDKTHTCPPCPAPELLGGPSVFLFPPKPKD |
| | | | | TLMISRTPEVTCVVVDVSHEDPEVKFNWYVDG |
| | | | | VEVHNAKTKPCEEQYGSTYRCVSVLTVLHQD |
| | | | | WLNGKEYKCKVSNKALPAPIEKTISKAKGQPR |
| | | | | EPQVYTLPPSREEMTKNQVSLTCLVKGFYPSDI |
| | | | | AVEWESNGQPENNYKTTPPVLDSDGSFFLYSK |
| | | | | LTVDKSRWQQGNVFSCSVMHEALHNHYTQKS |
| | | | | LSLSPGKGGGGSGGGGSGGGGSGGGGSGGGG |
| | | | | SGGGGSDKTHTCPPCPAPELLGGPSVFLFPPKP |
| | | | | KDTLMISRTPEVTCVVVDVSHEDPEVKFNWYV |
| | | | | DGVEVHNAKTKPCEEQYGSTYRCVSVLTVLH |
| | | | | QDWLNGKEYKCKVSNKALPAPIEKTISKAKGQ |
| | | | | PREPQVYTLPPSREEMTKNQVSLTCLVKGFYPS |
| | | | | DIAVEWESNGQPENNYKTTPPVLDSDGSFFLYS |
| | | | | KLTVDKSRWQQGNVFSCSVMHEALHNHYTQK |
| | | | | SLSLSPGK |
| 419. | PSMA 76-B10 x | artificial | Aa | QVQLVESGGGLVKPGESLRLSCAASGFTFSDY |
| | FL 4-E9 CC x I2C | | | YMYWVRQAPGKGLEWVAIISDGGYYTYYSDII |
| | xscFc - Full | | | KGRFTISRDNAKNSLYLQMNSLKAEDTAVYYC |
| | Sequence | | | ARGFPLLRHGAMDYWGQGTLVTVSSGGGGSG |
| | | | | GGGSGGGGSDIQMTQSPSSLSASVGDRVTITCK |
| | | | | ASQNVDTNVAWYQQKPGQAPKSLIYSASYRY |
| | | | | SDVPSRFSGSASGTDFTLTISSVQSEDFATYYCQ |
| | | | | QYDSYPYTFGGGTKLEIKSGGGGSQVTLKESG |
| | | | | PTLVKPTETLTLTCTVSGFSFRNARMGVSWIRQ |
| | | | | PPGKCLEWLAHIFSNDEKSYSTSLKSRLTISKDT |
| | | | | SKSQVVLTMTNMDPVDTATYFCARVPEYSSG |
| | | | | WYRFDYWGQGTLVTVSSGGGGSGGGGSGGG |
| | | | | GSDIQMTQSPSSLSASVGDRVTISCRASQSIRSY |
| | | | | LNWYQQKPGKAPKLLIYATSSLQGGVPSRFSG |
| | | | | SGSGTDFTLTISSLQPEDFATYYCQQSYSTPFTF |
| | | | | GCGTKVEIKSGGGGSEVQLVESGGGLVQPGGS |
| | | | | LKLSCAASGFTFNKYAMNWVRQAPGKGLEW |
| | | | | VARIRSKYNNYATYYADSVKDRFTISRDDSKN |
| | | | | TAYLQMNNLKTEDTAVYYCVRHGNFGNSYIS |
| | | | | YWAYWGQGTLVTVSSGGGGSGGGGSGGGGS |
| | | | | QTVVTQEPSLTVSPGGTVTLTCGSSTGAVTSGN |
| | | | | YPNWVQQKPGQAPRGLIGGTKFLAPGTPARFS |
| | | | | GSLLGGKAALTLSGVQPEDEAEYYCVLWYSN |
| | | | | RWVFGGGTKLTVLGGGGDKTHTCPPCPAPELL |
| | | | | GGPSVFLFPPKPKDTLMISRTPEVTCVVVDVSH |
| | | | | EDPEVKFNWYVDGVEVHNAKTKPCEEQYGST |
| | | | | YRCVSVLTVLHQDWLNGKEYKCKVSNKALPA |
| | | | | PIEKTISKAKGQPREPQVYTLPPSREEMTKNQV |
| | | | | SLTCLVKGFYPSDIAVEWESNGQPENNYKTTPP |
| | | | | VLDSDGSFFLYSKLTVDKSRWQQGNVFSCSVM |
| | | | | HEALHNHYTQKSLSLSPGKGGGGSGGGGSGG |
| | | | | GGSGGGGSGGGGSGGGGSDKTHTCPPCPAPEL |
| | | | | LGGPSVFLFPPKPKDTLMISRTPEVTCVVVDVS |
| | | | | HEDPEVKFNWYVDGVEVHNAKTKPCEEQYGS |
| | | | | TYRCVSVLTVLHQDWLNGKEYKCKVSNKALP |
| | | | | APIEKTISKAKGQPREPQVYTLPPSREEMTKNQ |
| | | | | VSLTCLVKGFYPSDIAVEWESNGQPENNYKTT |
| | | | | PPVLDSDGSFFLYSKLTVDKSRWQQGNVFSCS |
| | | | | VMHEALHNHYTQKSLSLSPGK |
| 420. | VH CDR1 CD3 | artificial | Aa | GFTFNKYAIN |
| | B2 | | | |
| 421. | VH CDR2 CD3 | artificial | Aa | RIRSKYNNYATYYADQVK |
| | B2 | | | |
| 422. | VH CDR3 CD3 | artificial | Aa | HANFGNSYISYWAY |
| | B2 | | | |
| 423. | VL CDR1 CD3 | artificial | Aa | ASSTGAVTSGNYPN |
| | B2 | | | |
| 424. | VL CDR2 CD3 | artificial | Aa | GTKFLVP |
| | B2 | | | |
| 425. | VL CDR3 CD3 | artificial | Aa | TLWYSNRWV |
| | B2 | | | |
| 426. | H VL CD3 B2 | artificial | Aa | EVQLVESGGGLVQPGGSLKLSCAASGFTENKY |
| | binder | | | AINWVRQAPGKGLEWVARIRSKYNNYATYYA |
| | | | | DQVKDRFTISRDDSKNTAYLQMNNLKTEDTA |
| | | | | VYYCVR |
| | | | | HANFGNSYISYWAYWGQGTLVTVSSGGGGSG |
| | | | | GGGSGGGGSQTVVTQEPSLTVSPGGTVTLTCA |
| | | | | SSTGAVTSGNYPNWVQQKPGQAPRGLIGGTKF |
| | | | | LVP |
| | | | | GTPARFSGSLLGGKAALTLSGVQPEDEAEYYC |
| | | | | TLWYSNRWVFGGGTKLTVL |
| 427. | CL1 9-G4 CC | artificial | Aa | QVQLVQSGAEVKKPGASVKVSCKASGYTFTD |
| | xI2Ccc xHSA xFL | | | YYMHWVRQAPGQCLEWMGWINPNSGGPNYA |
| | 4-E9 CC xI2Ccc | | | QKFQGRVTMTRDTSISTAHMELSRLRSDDTAV |
| | | | | YYCAREKHAVAGIGFDYWGQGTLVTVSSGGG |
| | | | | GSGGGGSGGGGSDIQMTQSPSSVSASVGDRVTI |
| | | | | TCQASQDISNYLNWYQQKPGKAPKLLIYAASS |
| | | | | LESGVPSRFSGSGSGTDFTLTISSLQPEDFATYY |
| | | | | CQQANSFPLTFGCGTKVDIKSGGGGSEVQLVE |
| | | | | SGGGLVQPGGSLKLSCAASGFTFNKYAMNWV |
| | | | | RQAPGKCLEWVARIRSKYNNYATYYADSVKD |
| | | | | RFTISRDDSKNTAYLQMNNLKTEDTAVYYCVR |
| | | | | HGNFGNSYISYWAYWGQGTLVTVSSGGGGSG |
| | | | | GGGSGGGGSQTVVTQEPSLTVSPGGTVTLTCG |
| | | | | SSTGAVTSGNYPNWVQQKPGQAPRGLIGGTKF |
| | | | | LAPGTPARFSGSLLGGKAALTLSGVQPEDEAE |
| | | | | YYCVLWYSNRWVFGCGTKLTVLGGGGSGGG |
| | | | | GSGGGGSDAHKSEVAHRFKDLGEENFKALVLI |
| | | | | AFAQYLQQCPFEDHVKLVNEVTEFAKTCVADE |
| | | | | SAENCDKSLHTLFGDKLCTVATLRETYGEMAD |
| | | | | CCAKQEPERNECFLQHKDDNPNLPRLVRPEVD |
| | | | | VMCTAFHDNEETFLKKYLYEIARRHPYFYAPE |
| | | | | LLFFAKRYKAAFTECCQAADKAACLLPKLDEL |
| | | | | RDEGKASSAKQRLKCASLQKFGERAFKAWAV |
| | | | | ARLSQRFPKAEFAEVSKLVTDLTKVHTECCHG |
| | | | | DLLECADDRADLAKYICENQDSISSKLKECCEK |
| | | | | PLLEKSHCIAEVENDEMPADLPSLAADFVESKD |
| | | | | VCKNYAEAKDVFLGMFLYEYARRHPDYSVVL |
| | | | | LLRLAKTYETTLEKCCAAADPHECYAKVFDEF |
| | | | | KPLVEEPQNLIKQNCELFEQLGEYKFQNALLVR |
| | | | | YTKKVPQVSTPTLVEVSRNLGKVGSKCCKHPE |
| | | | | AKRMPCAEDYLSVVLNQLCVLHEKTPVSDRV |
| | | | | TKCCTESLVNRRPCFSALEVDETYVPKEFNAET |
| | | | | FTFHADICTLSEKERQIKKQTALVELVKHKPKA |
| | | | | TKEQLKAVMDDFAAFVEKCCKADDKETCFAE |
| | | | | EGKKLVAASQAALGLGGGGSGGGGSGGGGSQ |
| | | | | VTLKESGPTLVKPTETLTLTCTVSGFSFRNARM |
| | | | | GVSWIRQPPGKCLEWLAHIFSNDEKSYSTSLKS |
| | | | | RLTISKDTSKSQVVLTMTNMDPVDTATYFCAR |
| | | | | VPEYSSGWYRFDYWGQGTLVTVSSGGGGSGG |
| | | | | GGSGGGGSDIQMTQSPSSLSASVGDRVTISCRA |
| | | | | SQSIRSYLNWYQQKPGKAPKLLIYATSSLQGG |
| | | | | VPSRFSGSGSGTDFTLTISSLQPEDFATYYCQQS |
| | | | | YSTPFTFGCGTKVEIKSGGGGSEVQLVESGGGL |
| | | | | VQPGGSLKLSCAASGFTFNKYAMNWVRQAPG |
| | | | | KCLEWVARIRSKYNNYATYYADSVKDRFTISR |
| | | | | DDSKNTAYLQMNNLKTEDTAVYYCVRHGNFG |
| | | | | NSYISYWAYWGQGTLVTVSSGGGGSGGGGSG |
| | | | | GGGSQTVVTQEPSLTVSPGGTVTLTCGSSTGA |
| | | | | VTSGNYPNWVQQKPGQAPRGLIGGTKFLAPGT |
| | | | | PARFSGSLLGGKAALTLSGVQPEDEAEYYCVL |
| | | | | WYSNRWVFGCGTKLTVL |
| 428. | CL1 9-G4 CC | artificial | Aa | QVQLVQSGAEVKKPGASVKVSCKASGYTFTD |
| | xI2Ccc xFL 4-E9 | | | YYMHWVRQAPGQCLEWMGWINPNSGGPNYA |
| | CC xI2Ccc | | | QKFQGRVTMTRDTSISTAHMELSRLRSDDTAV |
| | | | | YYCAREKHAVAGIGFDYWGQGTLVTVSSGGG |
| | | | | GSGGGGSGGGGSDIQMTQSPSSVSASVGDRVTI |
| | | | | TCQASQDISNYLNWYQQKPGKAPKLLIYAASS |
| | | | | LESGVPSRFSGSGSGTDFTLTISSLQPEDFATYY |
| | | | | CQQANSFPLTFGCGTKVDIKSGGGGSEVQLVE |
| | | | | SGGGLVQPGGSLKLSCAASGFTFNKYAMNWV |
| | | | | RQAPGKCLEWVARIRSKYNNYATYYADSVKD |
| | | | | RFTISRDDSKNTAYLQMNNLKTEDTAVYYCVR |
| | | | | HGNFGNSYISYWAYWGQGTLVTVSSGGGGSG |
| | | | | GGGSGGGGSQTVVTQEPSLTVSPGGTVTLTCG |
| | | | | SSTGAVTSGNYPNWVQQKPGQAPRGLIGGTKF |
| | | | | LAPGTPARFSGSLLGGKAALTLSGVQPEDEAE |
| | | | | YYCVLWYSNRWVFGCGTKLTVLGGGGSQVTL |
| | | | | KESGPTLVKPTETLTLTCTVSGFSFRNARMGVS |
| | | | | WIRQPPGKCLEWLAHIFSNDEKSYSTSLKSRLTI |
| | | | | SKDTSKSQVVLTMTNMDPVDTATYFCARVPE |
| | | | | YSSGWYRFDYWGQGTLVTVSSGGGGSGGGGS |
| | | | | GGGGSDIQMTQSPSSLSASVGDRVTISCRASQSI |
| | | | | RSYLNWYQQKPGKAPKLLIYATSSLQGGVPSR |
| | | | | FSGSGSGTDFTLTISSLQPEDFATYYCQQSYSTP |
| | | | | FTFGCGTKVEIKSGGGGSEVQLVESGGGLVQP |
| | | | | GGSLKLSCAASGFTFNKYAMNWVRQAPGKCL |
| | | | | EWVARIRSKYNNYATYYADSVKDRFTISRDDS |
| | | | | KNTAYLQMNNLKTEDTAVYYCVRHGNFGNSY |
| | | | | ISYWAYWGQGTLVTVSSGGGGSGGGGSGGGG |
| | | | | SQTVVTQEPSLTVSPGGTVTLTCGSSTGAVTSG |
| | | | | NYPNWVQQKPGQAPRGLIGGTKFLAPGTPARF |
| | | | | SGSLLGGKAALTLSGVQPEDEAEYYCVLWYSN |
| | | | | RWVFGCGTKLTVL |
| 429. | CL1 9-G4 CC | artificial | Aa | QVQLVQSGAEVKKPGASVKVSCKASGYTFTD |
| | xI2Ccc | | | YYMHWVRQAPGQCLEWMGWINPNSGGPNYA |
| | x(EAAAK)10xFL | | | QKFQGRVTMTRDTSISTAHMELSRLRSDDTAV |
| | 4-E9 CC xI2Ccc | | | YYCAREKHAVAGIGFDYWGQGTLVTVSSGGG |
| | | | | GSGGGGSGGGGSDIQMTQSPSSVSASVGDRVTI |
| | | | | TCQASQDISNYLNWYQQKPGKAPKLLIYAASS |
| | | | | LESGVPSRFSGSGSGTDFTLTISSLQPEDFATYY |
| | | | | CQQANSFPLTFGCGTKVDIKSGGGGSEVQLVE |
| | | | | SGGGLVQPGGSLKLSCAASGFTFNKYAMNWV |
| | | | | RQAPGKCLEWVARIRSKYNNYATYYADSVKD |
| | | | | RFTISRDDSKNTAYLQMNNLKTEDTAVYYCVR |
| | | | | HGNFGNSYISYWAYWGQGTLVTVSSGGGGSG |
| | | | | GGGSGGGGSQTVVTQEPSLTVSPGGTVTLTCG |
| | | | | SSTGAVTSGNYPNWVQQKPGQAPRGLIGGTKF |
| | | | | LAPGTPARFSGSLLGGKAALTLSGVQPEDEAE |
| | | | | YYCVLWYSNRWVFGCGTKLTVLEAAAKEAA |
| | | | | AKEAAAKEAAAKEAAAKEAAAKEAAAKEAA |
| | | | | AKEAAAKEAAAKQVTLKESGPTLVKPTETLTL |
| | | | | TCTVSGFSFRNARMGVSWIRQPPGKCLEWLAH |
| | | | | IFSNDEKSYSTSLKSRLTISKDTSKSQVVLTMTN |
| | | | | MDPVDTATYFCARVPEYSSGWYRFDYWGQGT |
| | | | | LVTVSSGGGGSGGGGSGGGGSDIQMTQSPSSL |
| | | | | SASVGDRVTISCRASQSIRSYLNWYQQKPGKA |
| | | | | PKLLIYATSSLQGGVPSRFSGSGSGTDFTLTISSL |
| | | | | QPEDFATYYCQQSYSTPFTFGCGTKVEIKSGGG |
| | | | | GSEVQLVESGGGLVQPGGSLKLSCAASGFTFN |
| | | | | KYAMNWVRQAPGKCLEWVARIRSKYNNYAT |
| | | | | YYADSVKDRFTISRDDSKNTAYLQMNNLKTED |
| | | | | TAVYYCVRHGNFGNSYISYWAYWGQGTLVTV |
| | | | | SSGGGGSGGGGSGGGGSQTVVTQEPSLTVSPG |
| | | | | GTVTLTCGSSTGAVTSGNYPNWVQQKPGQAP |
| | | | | RGLIGGTKFLAPGTPARFSGSLLGGKAALTLSG |
| | | | | VQPEDEAEYYCVLWYSNRWVFGCGTKLTVL |
| 430. | CL1 9-G4 CC | artificial | Aa | QVQLVQSGAEVKKPGASVKVSCKASGYTFTD |
| | xI2Ccc -scFc - | | | YYMHWVRQAPGQCLEWMGWINPNSGGPNYA |
| | scFc2 xFL 4-E9 | | | QKFQGRVTMTRDTSISTAHMELSRLRSDDTAV |
| | CC xI2Ccc | | | YYCAREKHAVAGIGFDYWGQGTLVTVSSGGG |
| | | | | GSGGGGSGGGGSDIQMTQSPSSVSASVGDRVTI |
| | | | | TCQASQDISNYLNWYQQKPGKAPKLLIYAASS |
| | | | | LESGVPSRFSGSGSGTDFTLTISSLQPEDFATYY |
| | | | | CQQANSFPLTFGCGTKVDIKSGGGGSEVQLVE |
| | | | | SGGGLVQPGGSLKLSCAASGFTFNKYAMNWV |
| | | | | RQAPGKCLEWVARIRSKYNNYATYYADSVKD |
| | | | | RFTISRDDSKNTAYLQMNNLKTEDTAVYYCVR |
| | | | | HGNFGNSYISYWAYWGQGTLVTVSSGGGGSG |
| | | | | GGGSGGGGSQTVVTQEPSLTVSPGGTVTLTCG |
| | | | | SSTGAVTSGNYPNWVQQKPGQAPRGLIGGTKF |
| | | | | LAPGTPARFSGSLLGGKAALTLSGVQPEDEAE |
| | | | | YYCVLWYSNRWVFGCGTKLTVLGGGGSGGG |
| | | | | GSGGGGSDKTHTCPPCPAPELLGGPSVFLFPPK |
| | | | | PKDTLMISRTPEVTCVVVDVSHEDPEVKFNWY |
| | | | | VDGVEVHNAKTKPCEEQYGSTYRCVSVLTVL |
| | | | | HQDWLNGKEYKCKVSNKALPAPIEKTISKAKG |
| | | | | QPREPQVYTLPPSREEMTKNQVSLTCLVKGFY |
| | | | | PSDIAVEWESNGQPENNYKTTPPVLDSDGSFFL |
| | | | | YSKLTVDKSRWQQGNVFSCSVMHEALHNHYT |
| | | | | QKSLSLSPGKGGGGSGGGGSGGGGSGGGGSG |
| | | | | GGGSGGGGSDKTHTCPPCPAPELLGGPSVFLFP |
| | | | | PKPKDTLMISRTPEVTCVVVDVSHEDPEVKEN |
| | | | | WYVDGVEVHNAKTKPCEEQYGSTYRCVSVLT |
| | | | | VLHQDWLNGKEYKCKVSNKALPAPIEKTISKA |
| | | | | KGQPREPQVYTLPPSREEMTKNQVSLTCLVKG |
| | | | | FYPSDIAVEWESNGQPENNYKTTPPVLDSDGSF |
| | | | | FLYSKLTVDKSRWQQGNVFSCSVMHEALHNH |
| | | | | YTQKSLSLSPGKDKTHTCPPCPAPELLGGPSVF |
| | | | | LFPPKPKDTLMISRTPEVTCVVVDVSHEDPEVK |
| | | | | FNWYVDGVEVHNAKTKPCEEQYGSTYRCVSV |
| | | | | LTVLHQDWLNGKEYKCKVSNKALPAPIEKTIS |
| | | | | KAKGQPREPQVYTLPPSREEMTKNQVSLTCLV |
| | | | | KGFYPSDIAVEWESNGQPENNYKTTPPVLDSD |
| | | | | GSFFLYSKLTVDKSRWQQGNVFSCSVMHEAL |
| | | | | HNHYTQKSLSLSPGKGGGGSGGGGSGGGGSG |
| | | | | GGGSGGGGSGGGGSDKTHTCPPCPAPELLGGP |
| | | | | SVFLFPPKPKDTLMISRTPEVTCVVVDVSHEDP |
| | | | | EVKFNWYVDGVEVHNAKTKPCEEQYGSTYRC |
| | | | | VSVLTVLHQDWLNGKEYKCKVSNKALPAPIEK |
| | | | | TISKAKGQPREPQVYTLPPSREEMTKNQVSLTC |
| | | | | LVKGFYPSDIAVEWESNGQPENNYKTTPPVLD |
| | | | | SDGSFFLYSKLTVDKSRWQQGNVFSCSVMHEA |
| | | | | LHNHYTQKSLSLSPGKGGGGSGGGGSGGGGS |
| | | | | QVTLKESGPTLVKPTETLTLTCTVSGFSFRNAR |
| | | | | MGVSWIRQPPGKCLEWLAHIFSNDEKSYSTSL |
| | | | | KSRLTISKDTSKSQVVLTMTNMDPVDTATYFC |
| | | | | ARVPEYSSGWYRFDYWGQGTLVTVSSGGGGS |
| | | | | GGGGSGGGGSDIQMTQSPSSLSASVGDRVTISC |
| | | | | RASQSIRSYLNWYQQKPGKAPKLLIYATSSLQG |
| | | | | GVPSRFSGSGSGTDFTLTISSLQPEDFATYYCQQ |
| | | | | SYSTPFTFGCGTKVEIKSGGGGSEVQLVESGGG |
| | | | | LVQPGGSLKLSCAASGFTFNKYAMNWVRQAP |
| | | | | GKCLEWVARIRSKYNNYATYYADSVKDRFTIS |
| | | | | RDDSKNTAYLQMNNLKTEDTAVYYCVRHGNF |
| | | | | GNSYISYWAYWGQGTLVTVSSGGGGSGGGGS |
| | | | | GGGGSQTVVTQEPSLTVSPGGTVTLTCGSSTG |
| | | | | AVTSGNYPNWVQQKPGQAPRGLIGGTKFLAPG |
| | | | | TPARFSGSLLGGKAALTLSGVQPEDEAEYYCV |
| | | | | LWYSNRWVFGCGTKLTVL |
| 431. | EpCAM 5-10 | artificial | Aa | ELVMTQSPSSLTVTAGEKVTMSCKSSQSLLNS |
| | xI2Ccc xHSA | | | GNQKNYLTWYQQKPGQPPKLLIYWASTRESG |
| | xH2 xI2Ccc | | | VPDRFTGSGSGTDFTLTISSVQAEDLAVYYCQN |
| | | | | DYSYPLTFGAGTKLEIKGGGGSGGGGSGGGGS |
| | | | | EVQLLEQSGAELVRPGTSVKISCKASGYAFTNY |
| | | | | WLGWVKQRPGHGLEWIGDIFPGSGNIHYNEKF |
| | | | | KGKATLTADKSSSTAYMQLSSLTFEDSAVYFC |
| | | | | ARLRNWDEPMDYWGQGTTVTVSSGGGGSEV |
| | | | | QLVESGGGLVQPGGSLKLSCAASGFTFNKYAM |
| | | | | NWVRQAPGKCLEWVARIRSKYNNYATYYADS |
| | | | | VKDRFTISRDDSKNTAYLQMNNLKTEDTAVY |
| | | | | YCVRHGNFGNSYISYWAYWGQGTLVTVSSGG |
| | | | | GGSGGGGSGGGGSQTVVTQEPSLTVSPGGTVT |
| | | | | LTCGSSTGAVTSGNYPNWVQQKPGQAPRGLIG |
| | | | | GTKFLAPGTPARFSGSLLGGKAALTLSGVQPED |
| | | | | EAEYYCVLWYSNRWVFGCGTKLTVLGGGGSG |
| | | | | GGGSGGGGSDAHKSEVAHRFKDLGEENFKAL |
| | | | | VLIAFAQYLQQCPFEDHVKLVNEVTEFAKTCV |
| | | | | ADESAENCDKSLHTLFGDKLCTVATLRETYGE |
| | | | | MADCCAKQEPERNECFLQHKDDNPNLPRLVRP |
| | | | | EVDVMCTAFHDNEETFLKKYLYEIARRHPYFY |
| | | | | APELLFFAKRYKAAFTECCQAADKAACLLPKL |
| | | | | DELRDEGKASSAKQRLKCASLQKFGERAFKA |
| | | | | WAVARLSQRFPKAEFAEVSKLVTDLTKVHTEC |
| | | | | CHGDLLECADDRADLAKYICENQDSISSKLKE |
| | | | | CCEKPLLEKSHCIAEVENDEMPADLPSLAADFV |
| | | | | ESKDVCKNYAEAKDVFLGMFLYEYARRHPDY |
| | | | | SVVLLLRLAKTYETTLEKCCAAADPHECYAKV |
| | | | | FDEFKPLVEEPQNLIKQNCELFEQLGEYKFQNA |
| | | | | LLVRYTKKVPQVSTPTLVEVSRNLGKVGSKCC |
| | | | | KHPEAKRMPCAEDYLSVVLNQLCVLHEKTPVS |
| | | | | DRVTKCCTESLVNRRPCFSALEVDETYVPKEF |
| | | | | NAETFTFHADICTLSEKERQIKKQTALVELVKH |
| | | | | KPKATKEQLKAVMDDFAAFVEKCCKADDKET |
| | | | | CFAEEGKKLVAASQAALGLGGGGSGGGGSGG |
| | | | | GGSEVQLLESGGGVVQPGRSLRLSCAASGFTFS |
| | | | | SYGMGWVRQAPGKGLEWVAVISYDGSNKYY |
| | | | | ADSVKGRFTISRDNSKNTLYLQMNSLRAEDTA |
| | | | | VYYCAREGAHFGSGSYYPLYYYYAMDVWGQ |
| | | | | GTTVTVSSGGGGSGGGGSGGGGSELTLTQSPG |
| | | | | TLSLSPGERATLSCRASQSVSSSYLAWYQQKPG |
| | | | | QAPRLLIYGASIRATGIPDRFSGSGSGTDFTLTIS |
| | | | | RLEPEDFAVYYCQQYGSSPIFTFGPGTKVEIKS |
| | | | | GGGGSEVQLVESGGGLVQPGGSLKLSCAASGF |
| | | | | TFNKYAMNWVRQAPGKCLEWVARIRSKYNN |
| | | | | YATYYADSVKDRFTISRDDSKNTAYLQMNNL |
| | | | | KTEDTAVYYCVRHGNFGNSYISYWAYWGQGT |
| | | | | LVTVSSGGGGSGGGGSGGGGSQTVVTQEPSLT |
| | | | | VSPGGTVTLTCGSSTGAVTSGNYPNWVQQKPG |
| | | | | QAPRGLIGGTKFLAPGTPARFSGSLLGGKAALT |
| | | | | LSGVQPEDEAEYYCVLWYSNRWVFGCGTKLT |
| | | | | VL |
| 432. | CH3 15- | artificial | Aa | NIAYGVAGTNYNQKFQG |
| | E11_1_VAG_CC - | | | |
| | HCDR2 | | | |
| 433. | CH3 15- | artificial | Aa | QVQLVQSGAEVKKPGASVKVSCKASGYTFTN |
| | E11_1_VAG_CC - | | | YWMNWVRQAPGQCLEWMGNIAYGVAGTNY |
| | VH | | | NQKFQGRVTMTVDTSSSTAYMELSRLRSDDTA |
| | | | | VYYCATRYFYVMDYWGQGTLVTVSS |
| 434. | CH3 15- | artificial | Aa | QVQLVQSGAEVKKPGASVKVSCKASGYTFTN |
| | E11_1_VAG_CC | | | YWMNWVRQAPGQCLEWMGNIAYGVAGTNY |
| | x I2L x G4 x scFc | | | NQKFQGRVTMTVDTSSSTAYMELSRLRSDDTA |
| | x G4 x MS 15- | | | VYYCATRYFYVMDYWGQGTLVTVSSGGGGQ |
| | B12 CC x I2L | | | GGGGQGGGGQDIQMTQSPSSLSASVGDRVTIT |
| | clipopt_DI | | | CRASQDISNYLNWYQQKPGKVPKLLIYYTSRL |
| | | | | HSGVPSRFSGSGSGTDFTLTISSLQPEDVATYYC |
| | | | | VQYAQFPLTFGCGTKVEIKSGGGGQEVQLVES |
| | | | | GGGLVQPGGSLKLSCAASGFTFNKYAMNWVR |
| | | | | QAPGKGMEWVARIRSKYNNYATYYADAVKD |
| | | | | RFTISRDDSKNTLYLQMNNLKTEDTAVYYCVR |
| | | | | AGNFGSSYISYFAYWGQGTLVTVSSGGGGQGG |
| | | | | GGQGGGGQQTVVTQEPSLTVSPGGTVTITCGS |
| | | | | STGAVTSGNYPNWIQKKPGQAPRGLIGGTKFL |
| | | | | APGTPARFSGSLEGGKAALTLSGVQPEDEAEY |
| | | | | YCVLYYSNRWVFGSGTKLTVLGGGGCPPCPAP |
| | | | | ELLGGPSVFLFPPKPKDTLMISRTPEVTCVVVD |
| | | | | VSHEEPEVKFNWYVDGVEVHNAKTKPCEEQY |
| | | | | GSTYRCVSVLTVLHQDWLNGKEYKCKVSNKA |
| | | | | LPAPIEKTISKAKGQPREPQVYTLPPSREEMTK |
| | | | | NQVSLTCLVKGFYPSDIAVEWESNGQPENNYK |
| | | | | TTPPVLDSDGSFFLYSKLTVDKSRWQQGNVFS |
| | | | | CSVMHEALHNHYTQKSLSLSPGKGGGGQGGG |
| | | | | GQGGGGQGGGGQGGGGQGGGGQCPPCPAPEL |
| | | | | LGGPSVFLFPPKPKDTLMISRTPEVTCVVVDVS |
| | | | | HEEPEVKFNWYVDGVEVHNAKTKPCEEQYGS |
| | | | | TYRCVSVLTVLHQDWLNGKEYKCKVSNKALP |
| | | | | APIEKTISKAKGQPREPQVYTLPPSREEMTKNQ |
| | | | | VSLTCLVKGFYPSDIAVEWESNGQPENNYKTT |
| | | | | PPVLDSDGSFFLYSKLTVDKSRWQQGNVFSCS |
| | | | | VMHEALHNHYTQKSLSLSPGKGGGGQVQLQE |
| | | | | SGPGLVKPSETLSLTCTVSGGSISSSSYFWGWIR |
| | | | | QPPGKCLEWIGNIYYSGSSNYNPSLKSRVTISV |
| | | | | DTSKNQFSLKLSSVTAADTAVYYCARLPRGDR |
| | | | | DAFDIWGQGTMVTVSSGGGGQGGGGQGGGG |
| | | | | QDIVMTQSPSSLSASVGDRVTITCRASQGISNY |
| | | | | LAWYQQKPGKVPKLLIYAASTLQSGVPSRFSG |
| | | | | SGSGTDFTLTISSLQPEDFATYYCQQSYSTPFTF |
| | | | | GCGTKVEIKSGGGGQEVQLVESGGGLVQPGGS |
| | | | | LKLSCAASGFTFNKYAMNWVRQAPGKGMEW |
| | | | | VARIRSKYNNYATYYADAVKDRFTISRDDSKN |
| | | | | TLYLQMNNLKTEDTAVYYCVRAGNFGSSYISY |
| | | | | FAYWGQGTLVTVSSGGGGQGGGGQGGGGQQ |
| | | | | TVVTQEPSLTVSPGGTVTITCGSSTGAVTSGNY |
| | | | | PNWIQKKPGQAPRGLIGGTKFLAPGTPARFSGS |
| | | | | LEGGKAALTLSGVQPEDEAEYYCVLYYSNRW |
| | | | | VFGSGTKLTVL |
| 435. | CH3 15- | artificial | Aa | QVQLVQSGAEVKKPGASVKVSCKASGYTFTN |
| | E11_1_VAG_CC | | | YWMNWVRQAPGQCLEWMGNIAYGVAGTNY |
| | x I2L x G4 x scFc | | | NQKFQGRVTMTVDTSSSTAYMELSRLRSDDTA |
| | x G4 x MS 15- | | | VYYCATRYFYVMDYWGQGTLVTVSSGGGGQ |
| | B12 CC x I2L | | | GGGGQGGGGQEIQMTQSPSSLSASVGDRVTIT |
| | clipopt_EI | | | CRASQDISNYLNWYQQKPGKVPKLLIYYTSRL |
| | | | | HSGVPSRFSGSGSGTDFTLTISSLQPEDVATYYC |
| | | | | VQYAQFPLTFGCGTKVEIKSGGGGQEVQLVES |
| | | | | GGGLVQPGGSLKLSCAASGFTFNKYAMNWVR |
| | | | | QAPGKGMEWVARIRSKYNNYATYYADAVKD |
| | | | | RFTISRDDSKNTLYLQMNNLKTEDTAVYYCVR |
| | | | | AGNFGSSYISYFAYWGQGTLVTVSSGGGGQGG |
| | | | | GGQGGGGQQTVVTQEPSLTVSPGGTVTITCGS |
| | | | | STGAVTSGNYPNWIQKKPGQAPRGLIGGTKFL |
| | | | | APGTPARFSGSLEGGKAALTLSGVQPEDEAEY |
| | | | | YCVLYYSNRWVFGSGTKLTVLGGGGCPPCPAP |
| | | | | ELLGGPSVFLFPPKPKDTLMISRTPEVTCVVVD |
| | | | | VSHEEPEVKFNWYVDGVEVHNAKTKPCEEQY |
| | | | | GSTYRCVSVLTVLHQDWLNGKEYKCKVSNKA |
| | | | | LPAPIEKTISKAKGQPREPQVYTLPPSREEMTK |
| | | | | NQVSLTCLVKGFYPSDIAVEWESNGQPENNYK |
| | | | | TTPPVLDSDGSFFLYSKLTVDKSRWQQGNVFS |
| | | | | CSVMHEALHNHYTQKSLSLSPGKGGGGQGGG |
| | | | | GQGGGGQGGGGQGGGGQGGGGQCPPCPAPEL |
| | | | | LGGPSVFLFPPKPKDTLMISRTPEVTCVVVDVS |
| | | | | HEEPEVKFNWYVDGVEVHNAKTKPCEEQYGS |
| | | | | TYRCVSVLTVLHQDWLNGKEYKCKVSNKALP |
| | | | | APIEKTISKAKGQPREPQVYTLPPSREEMTKNQ |
| | | | | VSLTCLVKGFYPSDIAVEWESNGQPENNYKTT |
| | | | | PPVLDSDGSFFLYSKLTVDKSRWQQGNVFSCS |
| | | | | VMHEALHNHYTQKSLSLSPGKGGGGQVQLQE |
| | | | | SGPGLVKPSETLSLTCTVSGGSISSSSYFWGWIR |
| | | | | QPPGKCLEWIGNIYYSGSSNYNPSLKSRVTISV |
| | | | | DTSKNQFSLKLSSVTAADTAVYYCARLPRGDR |
| | | | | DAFDIWGQGTMVTVSSGGGGQGGGGQGGGG |
| | | | | QEIVMTQSPSSLSASVGDRVTITCRASQGISNYL |
| | | | | AWYQQKPGKVPKLLIYAASTLQSGVPSRFSGS |
| | | | | GSGTDFTLTISSLQPEDFATYYCQQSYSTPFTFG |
| | | | | CGTKVEIKSGGGGQEVQLVESGGGLVQPGGSL |
| | | | | KLSCAASGFTFNKYAMNWVRQAPGKGMEWV |
| | | | | ARIRSKYNNYATYYADAVKDRFTISRDDSKNT |
| | | | | LYLQMNNLKTEDTAVYYCVRAGNFGSSYISYF |
| | | | | AYWGQGTLVTVSSGGGGQGGGGQGGGGQQT |
| | | | | VVTQEPSLTVSPGGTVTITCGSSTGAVTSGNYP |
| | | | | NWIQKKPGQAPRGLIGGTKFLAPGTPARFSGSL |
| | | | | EGGKAALTLSGVQPEDEAEYYCVLYYSNRWV |
| | | | | FGSGTKLTVL |
| 436. | I2M - HCDR1 | artificial | Aa | KYAMN |
| 437. | I2M - HCDR2 | artificial | Aa | RIRSKYNNYATYYADAVKD |
| 438. | I2M - HCDR3 | artificial | Aa | AGNFGTSYISYWAY |
| 439. | I2M - LCDR1 | artificial | Aa | GSSTGAVTSGNYPN |
| 440. | I2M - LCDR2 | artificial | Aa | GTKFLAP |
| 441. | I2M - LCDR3 | artificial | Aa | VLWYSNRWV |
| 442. | I2M - VH | artificial | Aa | EVQLVESGGGLVQPGGSLKLSCAASGFTFNKY |
| | | | | AMNWVRQAPGKGMEWVARIRSKYNNYATYY |
| | | | | ADAVKDRFTISRDDSKNTLYLQMNNLKTEDTA |
| | | | | VYYCVRAGNFGTSYISYWAYWGQGTLVTVSS |
| 443. | I2M - VL | artificial | Aa | QTVVTQEPSLTVSPGGTVTITCGSSTGAVTSGN |
| | | | | YPNWVQKKPGQAPRGLIGGTKFLAPGTPARFS |
| | | | | GSLSGGKAALTLSGVQPEDEAEYYCVLWYSN |
| | | | | RWVFGSGTKLTVL |
| 444. | IgG4 subtype | artificial | Aa | ESKYGPPCPSCP |
| | hinge | | | |
| 445. | IgG1 subtype | artificial | Aa | EPKSCDKTHTCPPCP |
| | hinge | | | |
| 446. | EpCAM_19124- | artificial | Aa | RYDMH |
| | A6_CC - HCDR1 | | | |
| 447 | EpCAM_19124- | artificial | Aa | IISYDGSNKYYGDAVKG |
| | A6_CC - HCDR2 | | | |
| 448. | EpCAM_19124- | artificial | Aa | RAGFQFDF |
| | A6_CC - HCDR3 | | | |
| 449. | EpCAM_19124- | artificial | Aa | TGTSSDVGGYNYVS |
| | A6_CC - LCDR1 | | | |
| 450. | EpCAM_19124- | artificial | Aa | DVSSRPS |
| | A6_CC - LCDR2 | | | |
| 451. | EpCAM_19124- | artificial | Aa | SSYTSSSTWV |
| | A6_CC - LCDR3 | | | |
| 452. | EpCAM_19124- | artificial | Aa | QVQLVESGGGVVQPGRSLRLSCAASGFTFSR |
| | A6_CC - VH | | | YDMHWVRQAPGQCLEWMAIISYDGSNKYYG |
| | | | | DAVKGRFTISRDNSRNTLYLQMNSLRAEDTAV |
| | | | | YHCVKRAGFQFDFWGQGTLVTVSS |
| 453. | EpCAM_19124- | artificial | Aa | QSALTQPPSVSGSPGQSITISCTGTSSDVGGY |
| | A6_CC - VL | | | NYVSWYQQHPGKAPKLMIYDVSSRPSGVSNR |
| | | | | FSGSKSGNTASLTISGLQAEDEADYYCSSYTS |
| | | | | SSTWVFGCGTKLTVL |
| 454. | EpCAM_19124- | artificial | Aa | DYGMH |
| | B5_CC - HCDR1 | | | |
| 455. | EpCAM_19124- | artificial | Aa | GISWNSGNIGYADSVKG |
| | B5_CC - HCDR2 | | | |
| 456. | EpCAM_19124- | artificial | Aa | PDCSSTSCYRGYYFDY |
| | B5_CC - HCDR3 | | | |
| 457. | EpCAM_19124- | artificial | Aa | GGNNIGSKSVH |
| | B5_CC - LCDR1 | | | |
| 458. | EpCAM_19124- | artificial | Aa | DVSDRPS |
| | B5_CC - LCDR2 | | | |
| 459. | EpCAM_19124- | artificial | Aa | QVWDSNTDHVV |
| | B5_CC - LCDR3 | | | |
| 460. | EpCAM_19124- | artificial | Aa | EVQLVESGGGLVQPGRSLRLSCAASGFTFDD |
| | B5_CC - VH | | | YGMHWVRQAPGKCLEWVSGISWNSGNIGYA |
| | | | | DSVKGRFTISRDNAKNSLYLQMNSLRAEDTAL |
| | | | | YYCAKPDCSSTSCYRGYYFDYWGQGTLVTVS |
| | | | | S |
| 461. | EpCAM_19124- | artificial | Aa | SYVLTQPASVSVAPGQTARITCGGNNIGSKSV |
| | B5_CC - VL | | | HWYQQKPGQAPILVVYDVSDRPSGIPERFSG |
| | | | | SNSGNTATLTISRVEAGDEADYYCQVWDSNT |
| | | | | DHVVFGCGTKLTVL |
| 462. | EpCAM_19124- | artificial | Aa | SYAII |
| | C5_CC - | | | |
| | HCDR1 | | | |
| 463. | EpCAM_19124- | artificial | Aa | GIIPMFGTANYAQKFQG |
| | C5_CC - | | | |
| | HCDR2 | | | |
| 464. | EpCAM_19124- | artificial | Aa | VSGTYHWGY |
| | C5_CC - | | | |
| | HCDR3 | | | |
| 465. | EpCAM_19124- | artificial | Aa | TGTSSDVGGYNYVS |
| | C5_CC - LCDR1 | | | |
| 466. | EpCAM_19124- | artificial | Aa | DVSARPS |
| | C5_CC - LCDR2 | | | |
| 467. | EpCAM_19124- | artificial | Aa | SSYISSTSLV |
| | C5_CC - LCDR3 | | | |
| 468. | EpCAM_19124- | artificial | Aa | QVQLVQSGAEVKKPGSSVKVSCKASGGTFSS |
| | C5_CC - VH | | | YAIIWVRQAPGQCLEWMGGIIPMFGTANYAQK |
| | | | | FQGRVTITADESTSTAYMELSSLRSEDTAVYY |
| | | | | CARVSGTYHWGYWGQGTLVTVSS |
| 469. | EpCAM_19124- | artificial | Aa | QSALTQPASVSGSPGQSITISCTGTSSDVGGY |
| | C5_CC - VL | | | NYVSWYQQHPGKAPKLMIYDVSARPSGVSNR |
| | | | | FSGSKSGNTASLTISGLQAEDEADYYCSSYIS |
| | | | | STSLVFGCGTKLTVL |
| 470. | EpCAM_19124- | artificial | Aa | NYDMN |
| | C7_N67Q_CC - | | | |
| | HCDR1 | | | |
| 471. | EpCAM_19124- | artificial | Aa | VISYDGSQKSYSDSVKG |
| | C7_N67Q_CC - | | | |
| | HCDR2 | | | |
| 472. | EpCAM_19124- | artificial | Aa | RGATPFDY |
| | C7_N67Q_CC - | | | |
| | HCDR3 | | | |
| 473. | EpCAM_19124- | artificial | Aa | TGTSNDVGGYNYVS |
| | C7_N67Q_CC - | | | |
| | LCDR1 | | | |
| 474. | EpCAM_19124- | artificial | Aa | DVSSRPS |
| | C7_N67Q_CC - | | | |
| | LCDR2 | | | |
| 475. | EpCAM_19124- | artificial | Aa | SSYARSRTFVA |
| | C7_N67Q_CC - | | | |
| | LCDR3 | | | |
| 476. | EpCAM_19124- | artificial | Aa | QVQLVESGGGVLQPGRSLRLSCAASGFTFRN |
| | C7_N67Q_CC - | | | YDMNWVRQVPGKCLEWVAVISYDGSQKSYS |
| | VH | | | DSVKGRFTISRDNSKNTLSLQMNSLRNEDTAV |
| | | | | YYCAKRGATPFDYWGQGTLVTVSS |
| 477. | EpCAM_19124- | artificial | Aa | QSALTQPASVSGSPGQSITISCTGTSNDVGGY |
| | C7_N67Q_CC - | | | NYVSWYQQHPGKAPKLMIYDVSSRPSGISNR |
| | VL | | | FSGSKSGNTASLTISGLQAEDEADYYCSSYAR |
| | | | | SRTFVAFGCGTKLTVL |
| 478. | EpCAM_19124- | artificial | Aa | NYDMN |
| | C7_S69Y_CC - | | | |
| | HCDR1 | | | |
| 479. | EpCAM_19124- | artificial | Aa | VISYDGSNKYYSDSVKG |
| | C7_S69Y_CC - | | | |
| | HCDR2 | | | |
| 480. | EpCAM_19124- | artificial | Aa | RGATPFDY |
| | C7_S69Y_CC - | | | |
| | HCDR3 | | | |
| 481. | EpCAM_19124- | artificial | Aa | TGTSNDVGGYNYVS |
| | C7_S69Y_CC - | | | |
| | LCDR1 | | | |
| 482. | EpCAM_19124- | artificial | Aa | DVSSRPS |
| | C7_S69Y_CC - | | | |
| | LCDR2 | | | |
| 483. | EpCAM_19124- | artificial | Aa | SSYARSRTFVA |
| | C7_S69Y_CC - | | | |
| | LCDR3 | | | |
| 484. | EpCAM_19124- | artificial | Aa | QVQLVESGGGVLQPGRSLRLSCAASGFTFRN |
| | C7_S69Y_CC - | | | YDMNWVRQVPGKCLEWVAVISYDGSNKYYS |
| | VH | | | DSVKGRFTISRDNSKNTLSLQMNSLRNEDTAV |
| | | | | YYCAKRGATPFDYWGQGTLVTVSS |
| 485. | EpCAM_19124- | artificial | Aa | QSALTQPASVSGSPGQSITISCTGTSNDVGGY |
| | C7_S69Y CC - | | | NYVSWYQQHPGKAPKLMIYDVSSRPSGISNR |
| | VL | | | FSGSKSGNTASLTISGLQAEDEADYYCSSYAR |
| | | | | SRTFVAFGCGTKLTVL |
| 486. | EpCAM_19124- | artificial | Aa | NYDMN |
| | D3_CC - | | | |
| | HCDR1 | | | |
| 487. | EpCAM_19124- | artificial | Aa | VISYDGSDKHYTDSVKG |
| | D3_CC - | | | |
| | HCDR2 | | | |
| 488. | EpCAM_19124- | artificial | Aa | RGATPVDY |
| | D3_CC - | | | |
| | HCDR3 | | | |
| 489. | EpCAM_19124- | artificial | Aa | KSSQSLLHSNGYNYLG |
| | D3_CC - LCDR1 | | | |
| 490. | EpCAM_19124- | artificial | Aa | FGSSRAS |
| | D3_CC - LCDR2 | | | |
| 491. | EpCAM_19124- | artificial | Aa | MQALQTPFT |
| | D3_CC - LCDR3 | | | |
| 492. | EpCAM_19124- | artificial | Aa | QVQLVESGGGVVQPGRSLRLSCAASGFTFRN |
| | D3_CC - VH | | | YDMNWVRQAPGKCLEWVAVISYDGSDKHYT |
| | | | | DSVKGRFTISRDNSKNTLYLQMNSLRTEDTAV |
| | | | | YYCAKRGATPVDYWGQGTLVTVSS |
| 493. | EpCAM_19124- | artificial | Aa | EIVMTQSPLSLPVTPGEPASISCKSSQSLLHSN |
| | D3_CC - VL | | | GYNYLGWYLQKPGQSPQLLIYFGSSRASGVP |
| | | | | DRFSGSGSGTDFTLKISGVEAEDVGVYYCMQ |
| | | | | ALQTPFTFGCGTKVDIK |
| 494. | EpCAM_19124- | artificial | Aa | SYAII |
| | F5_CC - HCDR1 | | | |
| 495. | EpCAM_19124- | artificial | Aa | GIIPIFGTANYAQKFQG |
| | F5_CC - HCDR2 | | | |
| 496. | EpCAM_19124- | artificial | Aa | VSGTYHWGY |
| | F5_CC - HCDR3 | | | |
| 497. | EpCAM_19124- | artificial | Aa | TGTSSDIGSFNLVS |
| | F5_CC - LCDR1 | | | |
| 498. | EpCAM_19124- | artificial | Aa | EGYKRPS |
| | F5_CC - LCDR2 | | | |
| 499. | EpCAM_19124- | artificial | Aa | SSYISSSTLV |
| | F5_CC - LCDR3 | | | |
| 500. | EpCAM_19124- | artificial | Aa | QVQLVQSGAEVKKPGSSVKVSCKVSGGTFSS |
| | F5_CC - VH | | | YAIIWVRQAPGQCLEWMGGIIPIFGTANYAQKF |
| | | | | QGRVTITADESTSTAYMELSSLRSDDTAVYYC |
| | | | | ARVSGTYHWGYWGQGTLVTVSS |
| 501. | EpCAM_19124- | artificial | Aa | QSALTQPPSASGSPGQSITISCTGTSSDIGSFN |
| | F5_CC - VL | | | LVSWYQQHPGKAPKLMIYEGYKRPSGVSDRF |
| | | | | SGSKSGNTASLTISGLQAEDEADYYCSSYISS |
| | | | | STLVFGCGTKLTVL |
| 502. | EpCAM_19124- | artificial | Aa | RYWMS |
| | G7_CC - | | | |
| | HCDR1 | | | |
| 503. | EpCAM_19124- | artificial | Aa | EINPDSSTINYTPSLKD |
| | G7_CC - | | | |
| | HCDR2 | | | |
| 504. | EpCAM_19124- | artificial | Aa | YPWFTY |
| | G7_CC - | | | |
| | HCDR3 | | | |
| 505. | EpCAM_19124- | artificial | Aa | RSSQSLVHSNGNTYLH |
| | G7_CC - LCDR1 | | | |
| 506. | EpCAM_19124- | artificial | Aa | KVSNRFS |
| | G7_CC - LCDR2 | | | |
| 507. | EpCAM_19124- | artificial | Aa | SQSTHVPFT |
| | G7_CC - LCDR3 | | | |
| 508. | EpCAM_19124- | artificial | Aa | EVQLVESGGGLVQPGGSLKLSCAASGFDFSR |
| | G7_CC - VH | | | YWMSWVRQAPGKCLEWIGEINPDSSTINYTP |
| | | | | SLKDKFIVSRDNAKNTLYLQMSKVRSEDTALY |
| | | | | YCARYPWFTYWGQGTLVTVSS |
| 509. | EpCAM_19124- | artificial | Aa | EIVMTQTPLSLPVSLGDQASISCRSSQSLVHS |
| | G7_CC - VL | | | NGNTYLHWYLQKPGQSPKLLIYKVSNRFSGV |
| | | | | PDRFSGSGSGTDFTLKISRVEAEDLGVYFCSQ |
| | | | | STHVPFTFGCGTKLEIK |
| 510. | EpCAM_19124- | artificial | Aa | NYDMN |
| | H1T69Y_CC - | | | |
| | HCDR1 | | | |
| 511. | EpCAM_19124- | artificial | Aa | VISYDGSNKYYTDSVKG |
| | H1T69Y_CC - | | | |
| | HCDR2 | | | |
| 512. | EpCAM_19124- | artificial | Aa | RGATPVDY |
| | H1T69Y_CC - | | | |
| | HCDR3 | | | |
| 513. | EpCAM_19124- | artificial | Aa | RSSQSLLHSNGYNYLG |
| | H1T69Y_CC - | | | |
| | LCDR1 | | | |
| 514. | EpCAM_19124- | artificial | Aa | LGSSRAS |
| | H1T69Y_CC - | | | |
| | LCDR2 | | | |
| 515. | EpCAM_19124- | artificial | Aa | MQALQTPFT |
| | H1T69Y_CC - | | | |
| | LCDR3 | | | |
| 516. | EpCAM_19124- | artificial | Aa | EVQLLESGGGLVQPGRSLRLSCAASGFTFRN |
| | H1T69Y_CC - | | | YDMNWVRQVPGKCLEWVAVISYDGSNKYYT |
| | VH | | | DSVKGRFTISRDNSKNTLYLQMNSLRTEDTAV |
| | | | | YYCAKRGATPVDYWGQGTLVTVSS |
| 517. | EpCAM_19124- | artificial | Aa | EIVMTQSPLSLPVTPGEPASISCRSSQSLLHSN |
| | H1T69Y_CC - | | | GYNYLGWYLQKPGQSPQLLIYLGSSRASGVP |
| | VL | | | DRFSGSGSGTDFTLKISRVEAEDVGVYYCMQ |
| | | | | ALQTPFTFGCGTKLEIK |
| 518. | EpCAM_19124- | artificial | Aa | NYDMN |
| | H1_N67Q_CC - | | | |
| | HCDR1 | | | |
| 519. | EpCAM_19124- | artificial | Aa | VISYDGSQKTYTDSVKG |
| | H1_N67Q_CC - | | | |
| | HCDR2 | | | |
| 520. | EpCAM_19124- | artificial | Aa | RGATPVDY |
| | H1_N67Q_CC - | | | |
| | HCDR3 | | | |
| 521. | EpCAM_19124- | artificial | Aa | RSSQSLLHSNGYNYLG |
| | H1_N67Q_CC - | | | |
| | LCDR1 | | | |
| 522. | EpCAM_19124- | artificial | Aa | LGSSRAS |
| | H1_N67Q_CC - | | | |
| | LCDR2 | | | |
| 523. | EpCAM_19124- | artificial | Aa | MQALQTPFT |
| | H1_N67Q_CC - | | | |
| | LCDR3 | | | |
| 524. | EpCAM_19124- | artificial | Aa | EVQLLESGGGLVQPGRSLRLSCAASGFTFRN |
| | H1_N67Q_CC - | | | YDMNWVRQVPGKCLEWVAVISYDGSQKTYT |
| | VH | | | DSVKGRFTISRDNSKNTLYLQMNSLRTEDTAV |
| | | | | YYCAKRGATPVDYWGQGTLVTVSS |
| 525. | EpCAM_19124- | artificial | Aa | EIVMTQSPLSLPVTPGEPASISCRSSQSLLHSN |
| | H1_N67Q_CC - | | | GYNYLGWYLQKPGQSPQLLIYLGSSRASGVP |
| | VL | | | DRFSGSGSGTDFTLKISRVEAEDVGVYYCMQ |
| | | | | ALQTPFTFGCGTKLEIK |
| 526. | EpCAM_19125- | artificial | Aa | RYDMH |
| | A6_CC - HCDR1 | | | |
| 527. | EpCAM_19125- | artificial | Aa | IISYDGSNKYYGDAVKG |
| | A6_CC - HCDR2 | | | |
| 528. | EpCAM_19125- | artificial | Aa | RAGFQFDF |
| | A6_CC - HCDR3 | | | |
| 529. | EpCAM_19125- | artificial | Aa | TGTSSDVGGYNYVS |
| | A6_CC - LCDR1 | | | |
| 530. | EpCAM_19125- | artificial | Aa | EVSKRPA |
| | A6_CC - LCDR2 | | | |
| 531. | EpCAM_19125- | artificial | Aa | SSYAGSNNWV |
| | A6_CC - LCDR3 | | | |
| 532. | EpCAM_19125- | artificial | Aa | QVQLVESGGGVVQPGRSLRLSCAASGFTFSR |
| | A6_CC - VH | | | YDMHWVRQAPGQCLEWMAIISYDGSNKYYG |
| | | | | DAVKGRFTISRDNSRNTLYLQMNSLRAEDTAV |
| | | | | YHCVKRAGFQFDFWGQGTLVTVSS |
| 533. | EpCAM_19125- | artificial | Aa | QSALTQPPSASGSPGQSVTISCTGTSSDVGG |
| | A6_CC - VL | | | YNYVSWYRQHPGKAPKLMIYEVSKRPAGVPD |
| | | | | RFSGSKSGNTASLTVSGLQAEDEADYYCSSY |
| | | | | AGSNNWVFGCGTKLTVL |
| 534. | EpCAM_19125- | artificial | Aa | RYDMN |
| | G6_C107A_CC - | | | |
| | HCDR1 | | | |
| 535. | EpCAM_19125- | artificial | Aa | FISYDGSNEDYPDAVKG |
| | G6_C107A_CC - | | | |
| | HCDR2 | | | |
| 536. | EpCAM_19125- | artificial | Aa | VGASPFDY |
| | G6_C107A_CC - | | | |
| | HCDR3 | | | |
| 537. | EpCAM_19125- | artificial | Aa | TGTSNDVGGYNYVS |
| | G6_C107A_CC - | | | |
| | LCDR1 | | | |
| 538. | EpCAM_19125- | artificial | Aa | EVSKRPS |
| | G6_C107A_CC - | | | |
| | LCDR2 | | | |
| 539. | EpCAM_19125- | artificial | Aa | ASYTGGRTYVG |
| | G6_C107A_CC - | | | |
| | LCDR3 | | | |
| 540. | EpCAM_19125- | artificial | Aa | QVQLVESGGGVVQPGRSLRLSCAASGFTFSR |
| | G6_C107A_CC - | | | YDMNWVRQAPGKCLEWVAFISYDGSNEDYP |
| | VH | | | DAVKGRFTISRDNSKNTLYLQMNSLRAEDTAV |
| | | | | YYCAKVGASPFDYWGQGTLVTVSS |
| 541. | EpCAM_19125- | artificial | Aa | QSALTQPPSVSGSPGQSITISCTGTSNDVGGY |
| | G6_C107A_CC - | | | NYVSWYQQHPGKAPKLMIYEVSKRPSGVPDR |
| | VL | | | FSGSKSGNTASLTISGLQAEDEADYYCASYTG |
| | | | | GRTYVGFGCGTKLTVL |
| 542. | EpCAM_19125- | artificial | Aa | RYDMN |
| | G6_C107L_CC - | | | |
| | HCDR1 | | | |
| 543. | EpCAM_19125- | artificial | Aa | FISYDGSNEDYPDAVKG |
| | G6_C107L_CC - | | | |
| | HCDR2 | | | |
| 544. | EpCAM_19125- | artificial | Aa | VGASPFDY |
| | G6_C107L_CC - | | | |
| | HCDR3 | | | |
| 545. | EpCAM_19125- | artificial | Aa | TGTSNDVGGYNYVS |
| | G6_C107L_CC - | | | |
| | LCDR1 | | | |
| 546. | EpCAM_19125- | artificial | Aa | EVSKRPS |
| | G6_C107L_CC - | | | |
| | LCDR2 | | | |
| 547. | EpCAM_19125- | artificial | Aa | LSYTGGRTYVG |
| | G6_C107L CC - | | | |
| | LCDR3 | | | |
| 548. | EpCAM_19125- | artificial | Aa | QVQLVESGGGVVQPGRSLRLSCAASGFTFSR |
| | G6_C107L_CC - | | | YDMNWVRQAPGKCLEWVAFISYDGSNEDYP |
| | VH | | | DAVKGRFTISRDNSKNTLYLQMNSLRAEDTAV |
| | | | | YYCAKVGASPFDYWGQGTLVTVSS |
| 549. | EpCAM_19125- | artificial | Aa | QSALTQPPSVSGSPGQSITISCTGTSNDVGGY |
| | G6_C107L_CC - | | | NYVSWYQQHPGKAPKLMIYEVSKRPSGVPDR |
| | VL | | | FSGSKSGNTASLTISGLQAEDEADYYCLSYTG |
| | | | | GRTYVGFGCGTKLTVL |
| 550. | EpCAM_19126- | artificial | Aa | TYTIS |
| | D5_CC - | | | |
| | HCDR1 | | | |
| 551. | EpCAM_19126- | artificial | Aa | GIIPILGAPNYAQKFQG |
| | D5_CC - | | | |
| | HCDR2 | | | |
| 552. | EpCAM_19126- | artificial | Aa | DPFSRY |
| | D5_CC - | | | |
| | HCDR3 | | | |
| 553. | EpCAM_19126- | artificial | Aa | RSSQSLLHSNGYNYLD |
| | D5_CC - LCDR1 | | | |
| 554. | EpCAM_19126- | artificial | Aa | LGSNRAS |
| | D5_CC - LCDR2 | | | |
| 555. | EpCAM_19126- | artificial | Aa | MQALQTPRT |
| | D5_CC - LCDR3 | | | |
| 556. | EpCAM_19126- | artificial | Aa | QVQLVQSGAEVKKPGSSVKVSCKVSGGTFST |
| | D5_CC - VH | | | YTISWVRQAPGQCLEWMGGIIPILGAPNYAQK |
| | | | | FQGRVSITADESTSTSYMELTSLRSEDTAVYY |
| | | | | CARDPFSRYWGQGTLVTVSS |
| 557. | EpCAM_19126- | artificial | Aa | EIVMTQSPLSLPVTPGEPASISCRSSQSLLHSN |
| | D5_CC - VL | | | GYNYLDWYLQKPGQSPQLLIYLGSNRASGVP |
| | | | | DRFSGSGSGTDFTLKISRVEAEDVGVYYCMQ |
| | | | | ALQTPRTFGCGTKVEIK |
| 558. | EpCAM_19127- | artificial | Aa | SYAII |
| | B6_CC - HCDR1 | | | |
| 559. | EpCAM_19127- | artificial | Aa | GIIPMFGTANYAQKFQG |
| | B6_CC - HCDR2 | | | |
| 560. | EpCAM_19127- | artificial | Aa | VSGTYHWGY |
| | B6_CC - HCDR3 | | | |
| 561. | EpCAM_19127- | artificial | Aa | TGTSSDVGGYNYVS |
| | B6_CC - LCDR1 | | | |
| 562. | EpCAM_19127- | artificial | Aa | DVSARPS |
| | B6_CC - LCDR2 | | | |
| 563. | EpCAM_19127- | artificial | Aa | SSYISITTLV |
| | B6_CC - LCDR3 | | | |
| 564. | EpCAM_19127- | artificial | Aa | QVQLVQSGAEVKKPGSSVKVSCKASGGTFRS |
| | B6_CC - VH | | | YAIIWVRQAPGQCLEWMGGIIPMFGTANYAQK |
| | | | | FQGRVTITADESTSTAYMELSRLRSEDTAVYY |
| | | | | CARVSGTYHWGYWGQGTLVTVSS |
| 565. | EpCAM_19127- | artificial | Aa | QSALTQPASVSGSPGQSITISCTGTSSDVGGY |
| | B6_CC - VL | | | NYVSWYQQRPGRAPKLMIYDVSARPSGVSNR |
| | | | | FSGSKSGNTASLTISGLQAEDEADYYCSSYISI |
| | | | | TTLVFGCGTKLTVL |
| 566. | EpCAM_19127- | artificial | Aa | RYDMH |
| | G11_CC - | | | |
| | HCDR1 | | | |
| 567. | EpCAM_19127- | artificial | Aa | IISYDGSIRYYADSVKG |
| | G11_CC - | | | |
| | HCDR2 | | | |
| 568. | EpCAM_19127- | artificial | Aa | RAGFQFDS |
| | G11_CC - | | | |
| | HCDR3 | | | |
| 569. | EpCAM_19127- | artificial | Aa | TGTSSDVGGYNYVS |
| | G11_CC - | | | |
| | LCDR1 | | | |
| 570. | EpCAM_19127- | artificial | Aa | EVSKRPA |
| | G11_CC - | | | |
| | LCDR2 | | | |
| 571. | EpCAM_19127- | artificial | Aa | SSYAGGNNFVV |
| | G11_CC - | | | |
| | LCDR3 | | | |
| 572. | EpCAM_19127- | artificial | Aa | QVQLVESGGGVVQPGRSLRLSCAASGFTFSR |
| | G11_CC - VH | | | YDMHWVRQAPGQCLEWMAIISYDGSIRYYAD |
| | | | | SVKGRFTISRDNSRNTLYLQMNSLRAEDTAVY |
| | | | | YCVKRAGFQFDSWGQGTLVTVSS |
| 573. | EpCAM_19127- | artificial | Aa | QSALTQPPSASGSPGQSVTISCTGTSSDVGG |
| | G11_CC - VL | | | YNYVSWYQQHPGKAPKLMIYEVSKRPAGVPD |
| | | | | RFSGSKSGNTASLTVSGLQAEDEADYYCSSY |
| | | | | AGGNNFVVFGCGTKLTVL |
| 574. | EpCAM_19128- | artificial | Aa | EYWMS |
| | H8_CC - | | | |
| | HCDR1 | | | |
| 575. | EpCAM_19128- | artificial | Aa | EIIPDSSKINYTPSLKD |
| | H8_CC - | | | |
| | HCDR2 | | | |
| 576. | EpCAM_19128- | artificial | Aa | PLYYGYDEGFAY |
| | H8_CC - | | | |
| | HCDR3 | | | |
| 577. | EpCAM_19128- | artificial | Aa | RSSQSLVHSNGNTYLE |
| | H8_CC - LCDR1 | | | |
| 578. | EpCAM_19128- | artificial | Aa | KVSNRFS |
| | H8_CC - LCDR2 | | | |
| 579. | EpCAM_19128- | artificial | Aa | FQGSHVPYT |
| | H8_CC - LCDR3 | | | |
| 580. | EpCAM_19128- | artificial | Aa | EVQLVESGGGLVQPGRSLKLSCAASGFDFSE |
| | H8_CC - VH | | | YWMSWVRQAPGKCLEWIGEIIPDSSKINYTPS |
| | | | | LKDKFIISRDNAKNTLYLQMSKVRSEDTALYYC |
| | | | | ARPLYYGYDEGFAYWGQGTTVTVSS |
| 581. | EpCAM_19128- | artificial | Aa | EIVMTQTPLSLPVSLGDQASISCRSSQSLVHS |
| | H8_CC - VL | | | NGNTYLEWYLQKPGQSPKLLIYKVSNRFSGV |
| | | | | PDRFSGSGSGTDFTLKISRVEAEDLGVYYCFQ |
| | | | | GSHVPYTFGCGTRLEIK |
| 582. | EpCAM_19129- | artificial | Aa | SYAMH |
| | A4_CC - HCDR1 | | | |
| 583. | EpCAM_19129- | artificial | Aa | RVRSKSDNYATYYADSVKD |
| | A4_CC - HCDR2 | | | |
| 584. | EpCAM_19129- | artificial | Aa | PLFTTVEVTNALDY |
| | A4_CC - HCDR3 | | | |
| 585. | EpCAM_19129- | artificial | Aa | SASSSISSNYLH |
| | A4_CC - LCDR1 | | | |
| 586. | EpCAM_19129- | artificial | Aa | RTSVLSS |
| | A4_CC - LCDR2 | | | |
| 587. | EpCAM_19129- | artificial | Aa | QQGSSMPFT |
| | A4_CC - LCDR3 | | | |
| 588. | EpCAM_19129- | artificial | Aa | EVQLVESGGGLVQPKGSLKLSCAASGFTFNS |
| | A4_CC - VH | | | YAMHWVRQAPGRCMEWVGRVRSKSDNYAT |
| | | | | YYADSVKDRFTISRDDSQSMLYLQMNNLKTE |
| | | | | DTAIYYCVRPLFTTVEVTNALDYWGQGTLVTV |
| | | | | SS |
| 589. | EpCAM_19129- | artificial | Aa | EIVLTQSPTTMAASPGEKITITCSASSSISSNYL |
| | A4_CC - VL | | | HWYQQKPGFSPKLLIYRTSVLSSGVPARFSG |
| | | | | SGSGTSYSLTIDTMEAEDVATYFCQQGSSMP |
| | | | | FTFGCGTRLEIK |
| 590. | EpCAM_19129- | artificial | Aa | NYWMQ |
| | E3_CC - HCDR1 | | | |
| 591. | EpCAM_19129- | artificial | Aa | AIYPGEGETRYTQKFKG |
| | E3_CC - HCDR2 | | | |
| 592. | EpCAM_19129- | artificial | Aa | PYAGYYLYAMDQ |
| | E3_CC - HCDR3 | | | |
| 593. | EpCAM_19129- | artificial | Aa | RSSQSIVHSNGNTYLE |
| | E3_CC - LCDR1 | | | |
| 594. | EpCAM_19129- | artificial | Aa | KVSNRFS |
| | E3_CC - LCDR2 | | | |
| 595. | EpCAM_19129- | artificial | Aa | SQSTHVPYT |
| | E3_CC - LCDR3 | | | |
| 596. | EpCAM_19129- | artificial | Aa | QVQLVQSGAELARPGASVKLSCKASGYIFSNY |
| | E3_CC - VH | | | WMQWVKQRPGQCLEWIGAIYPGEGETRYTQ |
| | | | | KFKGKATLTADTSSSTAYMQLSSLASEDSAVY |
| | | | | YCARPYAGYYLYAMDQWGQGTTVTVSS |
| 597. | EpCAM_19129- | artificial | Aa | EIVMTQTPLSLPVSLGDQASISCRSSQSIVHSN |
| | E3_CC - VL | | | GNTYLEWYLQKPGQSPKLLIYKVSNRFSGVP |
| | | | | DRFSGSGSGTDFTLKISRVEAEDLGVYFCSQS |
| | | | | THVPYTFGCGTRLEIK |
| 598. | EpCAM_19130- | artificial | Aa | NYDMN |
| | C11_CC - | | | |
| | HCDR1 | | | |
| 599. | EpCAM_19130- | artificial | Aa | VISYDGSNKYYTDSVKG |
| | C11_CC - | | | |
| | HCDR2 | | | |
| 600. | EpCAM_19130- | artificial | Aa | RGATPVDY |
| | C11_CC - | | | |
| | HCDR3 | | | |
| 601. | EpCAM_19130- | artificial | Aa | RSSQSLLHSNGYNYLG |
| | C11_CC - | | | |
| | LCDR1 | | | |
| 602. | EpCAM_19130- | artificial | Aa | FGSSRAS |
| | C11_CC - | | | |
| | LCDR2 | | | |
| 603. | EpCAM_19130- | artificial | Aa | MQALQTPFT |
| | C11_CC - | | | |
| | LCDR3 | | | |
| 604. | EpCAM_19130- | artificial | Aa | QVQLVESGGGVVQPGRSLRLSCAASGFTFSN |
| | C11_CC - VH | | | YDMNWVRQAPGKCLEWVAVISYDGSNKYYT |
| | | | | DSVKGRFTISRDNSKNTLYLQMNSLRTEDTAV |
| | | | | YYCAKRGATPVDYWGQGTLVTVSS |
| 605. | EpCAM_19130- | artificial | Aa | EIVMTQSPLSLPVTPGEPASISCRSSQSLLHSN |
| | C11_CC - VL | | | GYNYLGWYLQKPGQSPQLLIYFGSSRASGVP |
| | | | | DRFSGSGSGTDFTLKISGVEAEDVGVYYCMQ |
| | | | | ALQTPFTFGCGTKVDIK |
| 606. | EpCAM_19131- | artificial | Aa | RYDMH |
| | B6_CC - HCDR1 | | | |
| 607. | EpCAM_19131- | artificial | Aa | FISYDGSNEDYPDAVKG |
| | B6_CC - HCDR2 | | | |
| 608. | EpCAM_19131- | artificial | Aa | VGASPFDY |
| | B6_CC - HCDR3 | | | |
| 609. | EpCAM_19131- | artificial | Aa | TGTSSDVGGYNYVS |
| | B6_CC - LCDR1 | | | |
| 610. | EpCAM_19131- | artificial | Aa | EVSKRPS |
| | B6_CC - LCDR2 | | | |
| 611. | EpCAM_19131- | artificial | Aa | TSYAGSNNLV |
| | B6_CC - LCDR3 | | | |
| 612. | EpCAM_19131- | artificial | Aa | QVQLVESGGGVVQPGRSLRLSCAASGFTFSR |
| | B6_CC - VH | | | YDMHWVRQAPGKCLEWVAFISYDGSNEDYP |
| | | | | DAVKGRFTISRDNSKNTLYLQMNSLRAEDTAV |
| | | | | YYCAKVGASPFDYWGQGTLVTVSS |
| 613. | EpCAM_19131- | artificial | Aa | QSALTQPASVSGSPGRSVTISCTGTSSDVGG |
| | B6_CC - VL | | | YNYVSWYQQHPGKAPKLMIYEVSKRPSGVPV |
| | | | | RFSGSKSDNTASLTVSGLQAEDEADYYCTSY |
| | | | | AGSNNLVFGCGTKLTVL |
| 614. | EpCAM_19131- | artificial | Aa | NYDMN |
| | H3_hu_N67Q_C | | | |
| | C - HCDR1 | | | |
| 615. | EpCAM_19131- | artificial | Aa | VISYDGSQKSYSDSVKG |
| | H3_hu_N67Q_C | | | |
| | C - HCDR2 | | | |
| 616. | EpCAM_19131- | artificial | Aa | RGATPFDY |
| | H3_hu_N67Q_C | | | |
| | C - HCDR3 | | | |
| 617. | EpCAM_19131- | artificial | Aa | SGDKLGDKYAS |
| | H3_hu_N67Q_C | | | |
| | C - LCDR1 | | | |
| 618. | EpCAM_19131- | artificial | Aa | QDSRRPS |
| | H3_hu_N67Q_C | | | |
| | C - LCDR2 | | | |
| 619. | EpCAM_19131- | artificial | Aa | QVWDYSSDHWV |
| | H3_hu_N67Q_C | | | |
| | C - LCDR3 | | | |
| 620. | EpCAM_19131- | artificial | Aa | QVQLVESGGGVVQPGRSLRLSCAASGFTFRN |
| | H3_hu_N67Q_C | | | YDMNWVRQAPGKCLEWVAVISYDGSQKSYS |
| | C - VH | | | DSVKGRFTISRDNSKNTLSLQMNSLRNEDSAV |
| | | | | YYCAKRGATPFDYWGQGTLVTVSS |
| 621. | EpCAM_19131- | artificial | Aa | SYELTQPPSVSVSPGQTASITCSGDKLGDKYA |
| | H3_hu_N67Q_C | | | SWYQQKPGQSPVLVIYQDSRRPSGIPERFSG |
| | C - VL | | | SNSGNTATLTISGTQAMDEADYYCQVWDYSS |
| | | | | DHWVFGCGTKLTVL |
| 622. | EpCAM_19132- | artificial | Aa | NYDMN |
| | E12_hu_CC - | | | |
| | HCDR1 | | | |
| 623. | EpCAM_19132- | artificial | Aa | VISYDGSDKHYTDSVKG |
| | E12_hu_CC - | | | |
| | HCDR2 | | | |
| 624. | EpCAM_19132- | artificial | Aa | RGATPVDY |
| | E12_hu_CC - | | | |
| | HCDR3 | | | |
| 625. | EpCAM_19132- | artificial | Aa | SASSSISSNSLH |
| | E12_hu_CC - | | | |
| | LCDR1 | | | |
| 626. | EpCAM_19132- | artificial | Aa | RTSNLAS |
| | E12_hu_CC - | | | |
| | LCDR2 | | | |
| 627. | EpCAM_19132- | artificial | Aa | QQGSSIPRT |
| | E12_hu_CC - | | | |
| | LCDR3 | | | |
| 628. | EpCAM_19132- | artificial | Aa | QVQLVESGGGVVQPGRSLRLSCAASGFTFRN |
| | E12_hu_CC - | | | YDMNWVRQAPGKCLEWVAVISYDGSDKHYT |
| | VH | | | DSVKGRFTISRDNSKNTLFLQMNSLRTEDTAV |
| | | | | YYCAKRGATPVDYWGQGTLVTVSS |
| 629. | EpCAM_19132- | artificial | Aa | EIQMTQSPSSLSASVGDRVTITCSASSSISSNS |
| | E12_hu_CC - VL | | | LHWYQQKPGKAPKLLIYRTSNLASGVPSRFS |
| | | | | GSGSGTDFTLTISSLQPEDFATYYCQQGSSIP |
| | | | | RTFGCGTKLEIK |
| 630. | EpCAM_19143- | artificial | Aa | RYDMN |
| | C11_CC - | | | |
| | HCDR1 | | | |
| 631. | EpCAM_19143- | artificial | Aa | FISYDGSNEDYPDAVKG |
| | C11 CC - | | | |
| | HCDR2 | | | |
| 632. | EpCAM_19143- | artificial | Aa | VGASPFDY |
| | C11_CC - | | | |
| | HCDR3 | | | |
| 633. | EpCAM_19143- | artificial | Aa | RASQSVSSSYLA |
| | C11_CC - | | | |
| | LCDR1 | | | |
| 634. | EpCAM_19143- | artificial | Aa | GASSRAT |
| | C11_CC - | | | |
| | LCDR2 | | | |
| 635. | EpCAM_19143- | artificial | Aa | QQYGSSPRT |
| | C11_CC - | | | |
| | LCDR3 | | | |
| 636. | EpCAM_19143- | artificial | Aa | QVQLVESGGGVVQPGRSLRLSCAASGFTFSR |
| | C11_CC - VH | | | YDMNWVRQAPGKCLEWVAFISYDGSNEDYP |
| | | | | DAVKGRFTISRDNSKNTLYLQLNSLRAEDTAV |
| | | | | YYCAKVGASPFDYWGQGTLVTVSS |
| 637. | EpCAM_19143- | artificial | Aa | EIVLTQSPGTLSLSPGERATLSCRASQSVSSS |
| | C11_CC - VL | | | YLAWYQQKPGQAPRLLIYGASSRATGIPDRFS |
| | | | | GSGSGTDFTLTISRLEPEDFAVYYCQQYGSSP |
| | | | | RTFGCGTKVEIK |
| 638. | EpCAM_19143- | artificial | Aa | NYDMN |
| | E11_CC - | | | |
| | HCDR1 | | | |
| 639. | EpCAM_19143- | artificial | Aa | VISYDGSNKYYTDSVKG |
| | E11_CC - | | | |
| | HCDR2 | | | |
| 640. | EpCAM_19143- | artificial | Aa | RGATPFDY |
| | E11_CC - | | | |
| | HCDR3 | | | |
| 641. | EpCAM_19143- | artificial | Aa | RASQSVNSNLA |
| | E11_CC - | | | |
| | LCDR1 | | | |
| 642. | EpCAM_19143- | artificial | Aa | GASTRAT |
| | E11_CC - | | | |
| | LCDR2 | | | |
| 643. | EpCAM_19143- | artificial | Aa | QQYNNWPYT |
| | E11_CC - | | | |
| | LCDR3 | | | |
| 644. | EpCAM_19143- | artificial | Aa | QVQLVESGGGVVLPGRSLRLSCAASGFTFRN |
| | E11_CC - VH | | | YDMNWVRQAPGKCLEWVAVISYDGSNKYYT |
| | | | | DSVKGRFTISRDNSRNTLYLQMNSLRTEDTAV |
| | | | | YSCTKRGATPFDYWGQGTLVTVSS |
| 645. | EpCAM_19143- | artificial | Aa | EIVLTQSPATLSVSPGERATLSCRASQSVNSN |
| | E11_CC - VL | | | LAWYQQKPGQAPRLLIYGASTRATGIPARFSG |
| | | | | SGSGTEFTLTISSLQSEDFAVYYCQQYNNWP |
| | | | | YTFGCGTKLEIK |
| 646. | EpCAM_19145- | artificial | Aa | NYDMN |
| | C4_CC - | | | |
| | HCDR1 | | | |
| 647. | EpCAM_19145- | artificial | Aa | VISYDGSDKHYTDSVKG |
| | C4_CC - | | | |
| | HCDR2 | | | |
| 648. | EpCAM_19145- | artificial | Aa | RGATPVDY |
| | C4_CC - | | | |
| | HCDR3 | | | |
| 649. | EpCAM_19145- | artificial | Aa | RSSQSLLHSNGYNYLD |
| | C4_CC - LCDR1 | | | |
| 650. | EpCAM_19145- | artificial | Aa | LGSNRAS |
| | C4_CC - LCDR2 | | | |
| 651. | EpCAM_19145- | artificial | Aa | MQALQAPLT |
| | C4_CC - LCDR3 | | | |
| 652. | EpCAM_19145- | artificial | Aa | QVQLVESGGGVVQPGRSLRLSCAASGFTFRN |
| | C4_CC - VH | | | YDMNWVRQAPGKCLEWVAVISYDGSDKHYT |
| | | | | DSVKGRFTISRDNSKNTLYLQMNSLRTEDTAV |
| | | | | YYCAKRGATPVDYWGQGTLVTVSS |
| 653. | EpCAM_19145- | artificial | Aa | EIVMTQTPLSLPVTPGEPASISCRSSQSLLHSN |
| | C4_CC - VL | | | GYNYLDWYLQKPGQSPQLLIYLGSNRASGVP |
| | | | | DRFSGSGSGTDFTLKISRVEAEDVGVYYCMQ |
| | | | | ALQAPLTFGCGTKVDIK |
| 654. | EpCAM_19145- | artificial | Aa | RYDMN |
| | F12_CC - | | | |
| | HCDR1 | | | |
| 655. | EpCAM_19145- | artificial | Aa | FISYDGSNEDYPDAVKG |
| | F12_CC - | | | |
| | HCDR2 | | | |
| 656. | EpCAM_19145- | artificial | Aa | VGASPFDY |
| | F12_CC - | | | |
| | HCDR3 | | | |
| 657. | EpCAM_19145- | artificial | Aa | RSSQSLLHSNGYNYLG |
| | F12_CC - | | | |
| | LCDR1 | | | |
| 658. | EpCAM_19145- | artificial | Aa | SGSSRAS |
| | F12_CC - | | | |
| | LCDR2 | | | |
| 659. | EpCAM_19145- | artificial | Aa | MQALQTPFT |
| | F12_CC - | | | |
| | LCDR3 | | | |
| 660. | EpCAM_19145- | artificial | Aa | QVQLVESGGGVVQPGRSLRLSCAASGFTFSR |
| | F12_CC - VH | | | YDMNWVRQAPGKCLEWVAFISYDGSNEDYP |
| | | | | DAVKGRFTISRDNSKNTLYLQMNSLRAEDTAV |
| | | | | YYCAKVGASPFDYWGQGTLVTVSS |
| 661. | EpCAM_19145- | artificial | Aa | EIVMTQSPLSLPVTPGEPASISCRSSQSLLHSN |
| | F12_CC - VL | | | GYNYLGWYLQKPGQSPQLLIYSGSSRASGVP |
| | | | | DRFSGSGSGTDFTLKISRVEAEDVGVYYCMQ |
| | | | | ALQTPFTFGCGTKVEIK |
| 662. | EpCAM_19168- | artificial | Aa | RYYMH |
| | H9_CC - | | | |
| | HCDR1 | | | |
| 663. | EpCAM_19168- | artificial | Aa | VIWHDGSNKYYADSVKG |
| | H9_CC - | | | |
| | HCDR2 | | | |
| 664. | EpCAM_19168- | artificial | Aa | EAPSLAY |
| | H9_CC - | | | |
| | HCDR3 | | | |
| 665. | EpCAM_19168- | artificial | Aa | RASQSVSSSYLA |
| | H9_CC - LCDR1 | | | |
| 666. | EpCAM_19168- | artificial | Aa | GASSRAT |
| | H9_CC - LCDR2 | | | |
| 667. | EpCAM_19168- | artificial | Aa | QQYGSSPLT |
| | H9_CC - LCDR3 | | | |
| 668. | EpCAM_19168- | artificial | Aa | QVQLVESGGGVVQPGRSLRLSCAASGFTFSR |
| | H9_CC - VH | | | YYMHWVRQAPGKCPEWVAVIWHDGSNKYYA |
| | | | | DSVKGRFTISRDNSKNTLYLQMNSLRAEDTAV |
| | | | | YYCAREAPSLAYWGQGTLVTVSS |
| 669. | EpCAM_19168- | artificial | Aa | EIVLTQSPGTLSLSPGERATLSCRASQSVSSS |
| | H9_CC - VL | | | YLAWYQQKPGQAPRLLIYGASSRATGIPDRFS |
| | | | | GSGSGTDFTLTISRLEPEDFAVYYCQQYGSSP |
| | | | | LTFGCGTKVEIK |
| 670. | EpCAM_19171- | artificial | Aa | RYYMH |
| | A5_CC - HCDR1 | | | |
| 671. | EpCAM_19171- | artificial | Aa | VIWHDGSNKYYADSVKG |
| | A5_CC - HCDR2 | | | |
| 672. | EpCAM_19171- | artificial | Aa | EAPSLAY |
| | A5_CC - HCDR3 | | | |
| 673. | EpCAM_19171- | artificial | Aa | RASQSVSSSYLA |
| | A5_CC - LCDR1 | | | |
| 674. | EpCAM_19171- | artificial | Aa | GASSRAT |
| | A5_CC - LCDR2 | | | |
| 675. | EpCAM_19171- | artificial | Aa | QQYGSSIT |
| | A5_CC - LCDR3 | | | |
| 676. | EpCAM_19171- | artificial | Aa | QVQLVESGGGVVQPGRSLRLSCAASGFTFSR |
| | A5_CC - VH | | | YYMHWVRQAPGKCPEWVAVIWHDGSNKYYA |
| | | | | DSVKGRFTISRDNSKNTLYLQMNSLRAEDTAV |
| | | | | YYCAREAPSLAYWGQGTLVTVSS |
| 677. | EpCAM_19171- | artificial | Aa | EIVMTQSPGTLSLSPGERATLSCRASQSVSSS |
| | A5_CC - VL | | | YLAWYQQKPGQAPRLLIYGASSRATGIPDRFS |
| | | | | GSGSGTDFTLTISRLEPEDFAVYYCQQYGSSI |
| | | | | TFGCGTRLEIK |
| 678. | EpCAM_19171- | artificial | Aa | RYYMH |
| | D3_CC - | | | |
| | HCDR1 | | | |
| 679. | EpCAM_19171- | artificial | Aa | VIWHDGSNKYYADSVKG |
| | D3_CC - | | | |
| | HCDR2 | | | |
| 680. | EpCAM_19171- | artificial | Aa | EAPSLAY |
| | D3_CC - | | | |
| | HCDR3 | | | |
| 681. | EpCAM_19171- | artificial | Aa | RASQSVSSSYLA |
| | D3_CC - LCDR1 | | | |
| 682. | EpCAM_19171- | artificial | Aa | GASSRAT |
| | D3_CC - LCDR2 | | | |
| 683. | EpCAM_19171- | artificial | Aa | QQYGSSPWT |
| | D3_CC - LCDR3 | | | |
| 684. | EpCAM_19171- | artificial | Aa | QVQLVESGGGVVQPGRSLRLSCAASGFTFSR |
| | D3_CC - VH | | | YYMHWVRQAPGKCPEWVAVIWHDGSNKYYA |
| | | | | DSVKGRFTISRDNSKNTLYLQMNSLRAEDTAV |
| | | | | YYCAREAPSLAYWGQGTLVTVSS |
| 685. | EpCAM_19171- | artificial | Aa | EIVLTQSPGTLSLSPGERATLSCRASQSVSSS |
| | D3_CC - VL | | | YLAWYQQKPGQAPRLLIYGASSRATGIPDRFS |
| | | | | GSGSGTDFTLTISRLEPEDFAVYYCQQYGSSP |
| | | | | WTFGCGTKVEIK |
| 686. | EpCAM 19171- | artificial | Aa | SYYWS |
| | E11_CC - | | | |
| | HCDR1 | | | |
| 687. | EpCAM_19171- | artificial | Aa | RVYTSGSTDYNPSLKS |
| | E11_CC - | | | |
| | HCDR2 | | | |
| 688. | EpCAM_19171- | artificial | Aa | DSGNFWGFLDH |
| | E11_CC - | | | |
| | HCDR3 | | | |
| 689. | EpCAM_19171- | artificial | Aa | RSSQSLLHSNGYNYLD |
| | E11_CC - | | | |
| | LCDR1 | | | |
| 690. | EpCAM_19171- | artificial | Aa | LGSNRAS |
| | E11_CC - | | | |
| | LCDR2 | | | |
| 691. | EpCAM_19171- | artificial | Aa | MQALQTPWT |
| | E11_CC - | | | |
| | LCDR3 | | | |
| 692. | EpCAM_19171- | artificial | Aa | QVQLQESGPGLVKPSETLSLTCTVSGGSISSY |
| | E11_CC - VH | | | YWSWIRQPAGKCLEWIGRVYTSGSTDYNPSL |
| | | | | KSRVTMSLDTSKSQFSLKLRSVTAADTAVYYC |
| | | | | ARDSGNFWGFLDHWGQGTLVTVSS |
| 693. | EpCAM_19171- | artificial | Aa | EIVLTQSPLSLPVTPGEPASISCRSSQSLLHSN |
| | E11_CC - VL | | | GYNYLDWYLQKPGQSPQLLIYLGSNRASGVP |
| | | | | DRFSGSGSGTDFTLKISRVEAEDVGIYYCMQA |
| | | | | LQTPWTFGCGTKVEIK |
| 694. | EpCAM_19180- | artificial | Aa | NYDMN |
| | B12_CC - | | | |
| | HCDR1 | | | |
| 695. | EpCAM_19180- | artificial | Aa | VISYDGSNKYYTDSVKG |
| | B12_CC - | | | |
| | HCDR2 | | | |
| 696. | EpCAM_19180- | artificial | Aa | RGATPFDY |
| | B12_CC - | | | |
| | HCDR3 | | | |
| 697. | EpCAM_19180- | artificial | Aa | TGTNSDVGSYNLVS |
| | B12_CC - | | | |
| | LCDR1 | | | |
| 698. | EpCAM_19180- | artificial | Aa | DVSHRPS |
| | B12_CC - | | | |
| | LCDR2 | | | |
| 699. | EpCAM_19180- | artificial | Aa | SSYISSSSLV |
| | B12_CC - | | | |
| | LCDR3 | | | |
| 700. | EpCAM_19180- | artificial | Aa | EVQLLESGGGLVQPGGSLRLSCAASGFTFRN |
| | B12_CC - VH | | | YDMNWVRQAPGKCLEWVAVISYDGSNKYYT |
| | | | | DSVKGRFTISRDNSRNTLYLQMNSLRTEDTAV |
| | | | | YSCTKRGATPFDYWGQGTLVTVSS |
| 701. | EpCAM_19180- | artificial | Aa | QSALTQPPSVSGSPGQSITISCTGTNSDVGSY |
| | B12_CC - VL | | | NLVSWYQQHPGKTPKLMIYDVSHRPSGVSNR |
| | | | | FSGSKSGNTASLTISGLQAEDEADYYCSSYIS |
| | | | | SSSLVFGCGTKLTVL |
| 702. | EpCAM_19180- | artificial | Aa | NYDMN |
| | B6_N67Q_CC - | | | |
| | HCDR1 | | | |
| 703. | EpCAM_19180- | artificial | Aa | VISYDGSQKSYSDSVKG |
| | B6_N67Q_CC - | | | |
| | HCDR2 | | | |
| 704. | EpCAM_19180- | artificial | Aa | RGATPFDY |
| | B6_N67Q_CC - | | | |
| | HCDR3 | | | |
| 705. | EpCAM_19180- | artificial | Aa | GGNNIGSKNVH |
| | B6_N67Q_CC - | | | |
| | LCDR1 | | | |
| 706. | EpCAM_19180- | artificial | Aa | RDSKRPS |
| | B6_N67Q_CC - | | | |
| | LCDR2 | | | |
| 707. | EpCAM_19180- | artificial | Aa | QAWDRSTAV |
| | B6_N67Q_CC - | | | |
| | LCDR3 | | | |
| 708. | EpCAM_19180- | artificial | Aa | EVQLLESGGGSAQPGGSLRLSCVASGFTFRN |
| | B6_N67Q_CC - | | | YDMNWVRQAPGKCLEWVAVISYDGSQKSYS |
| | VH | | | DSVKGRFTISRDNSKNTLSLQMNSLRNEDTAV |
| | | | | YYCAKRGATPFDYWGQGTLVTVSS |
| 709. | EpCAM_19180- | artificial | Aa | SYELTQPPSVSVAPGQTARITCGGNNIGSKNV |
| | B6_N67Q_CC - | | | HWYQQKPGQAPVLVIYRDSKRPSGIPERFSG |
| | VL | | | SNSGNTATLTISGTQAMDEADYYCQAWDRST |
| | | | | AVFGCGTKLTVL |
| 710. | EpCAM_19180- | artificial | Aa | NYDMN |
| | D10S69Y_CC - | | | |
| | HCDR1 | | | |
| 711. | EpCAM_19180- | artificial | Aa | VISYDGSNKYYSDSVKG |
| | D10S69Y_CC - | | | |
| | HCDR2 | | | |
| 712. | EpCAM_19180- | artificial | Aa | RGATPFDY |
| | D10S69Y_CC - | | | |
| | HCDR3 | | | |
| 713. | EpCAM_19180- | artificial | Aa | TGTSSDVGGYNYVS |
| | D10S69Y_CC - | | | |
| | LCDR1 | | | |
| 714. | EpCAM_19180- | artificial | Aa | DVSVRPS |
| | D10S69Y_CC - | | | |
| | LCDR2 | | | |
| 715. | EpCAM_19180- | artificial | Aa | SSYISSTTLV |
| | D10S69Y_CC - | | | |
| | LCDR3 | | | |
| 716. | EpCAM_19180- | artificial | Aa | QVQLVESGGGVVQPGRSLRLSCAASGFTFRN |
| | D10S69Y_CC - | | | YDMNWVRQAPGKCLEWVAVISYDGSNKYYS |
| | VH | | | DSVKGRFTISRDNSKNTLSLQMNSLRNEDTAV |
| | | | | YYCAKRGATPFDYWGQGTLVTVSS |
| 717. | EpCAM_19180- | artificial | Aa | QSALTQPPSASGSPGQSITISCTGTSSDVGGY |
| | D10S69Y_CC - | | | NYVSWYQQHPGKAPKLMIYDVSVRPSGVSNR |
| | VL | | | FSGSKSGNTASLTISGLQAEDEADYYCSSYIS |
| | | | | STTLVFGCGTKLTVL |
| 718. | EpCAM_19180- | artificial | Aa | NYDMN |
| | D10_N67Q_CC - | | | |
| | HCDR1 | | | |
| 719. | EpCAM_19180- | artificial | Aa | VISYDGSQKSYSDSVKG |
| | D10_N67Q_CC - | | | |
| | HCDR2 | | | |
| 720. | EpCAM_19180- | artificial | Aa | RGATPFDY |
| | D10_N67Q_CC - | | | |
| | HCDR3 | | | |
| 721. | EpCAM_19180- | artificial | Aa | TGTSSDVGGYNYVS |
| | D10_N67Q_CC- | | | |
| | LCDR1 | | | |
| 722. | EpCAM_19180- | artificial | Aa | DVSVRPS |
| | D10_N67Q_CC- | | | |
| | LCDR2 | | | |
| 723. | EpCAM_19180- | artificial | Aa | SSYISSTTLV |
| | D10_N67Q_CC- | | | |
| | LCDR3 | | | |
| 724. | EpCAM_19180- | artificial | Aa | QVQLVESGGGVVQPGRSLRLSCAASGFTFRN |
| | D10_N67Q_CC- | | | YDMNWVRQAPGKCLEWVAVISYDGSQKSYS |
| | VH | | | DSVKGRFTISRDNSKNTLSLQMNSLRNEDTAV |
| | | | | YYCAKRGATPFDYWGQGTLVTVSS |
| 725. | EpCAM_19180- | artificial | Aa | QSALTQPPSASGSPGQSITISCTGTSSDVGGY |
| | D10_N67Q_CC- | | | NYVSWYQQHPGKAPKLMIYDVSVRPSGVSNR |
| | VL | | | FSGSKSGNTASLTISGLQAEDEADYYCSSYIS |
| | | | | STTLVFGCGTKLTVL |
| 726. | EpCAM_19180- | artificial | Aa | GYYMH |
| | G7_CC - | | | |
| | HCDR1 | | | |
| 727. | EpCAM_19180- | artificial | Aa | WINPNSGGTNYAQKFQG |
| | G7_CC - | | | |
| | HCDR2 | | | |
| 728. | EpCAM_19180- | artificial | Aa | TGALAGALKH |
| | G7_CC - | | | |
| | HCDR3 | | | |
| 729. | EpCAM_19180- | artificial | Aa | RSSQSLLHSNGYNYLD |
| | G7_CC - LCDR1 | | | |
| 730. | EpCAM_19180- | artificial | Aa | LGSNRAS |
| | G7_CC - LCDR2 | | | |
| 731. | EpCAM_19180- | artificial | Aa | MQALQTPFT |
| | G7_CC - LCDR3 | | | |
| 732. | EpCAM_19180- | artificial | Aa | QVQLVQSGAEVKKPGASVKVSCKASGYTFTG |
| | G7_CC - VH | | | YYMHWVRQAPGQCLEWMGWINPNSGGTNY |
| | | | | AQKFQGRITMTRDTSISTAYMELSRLRSDDTA |
| | | | | VYYCARTGALAGALKHWGQGTLVTVSS |
| 733. | EpCAM_19180- | artificial | Aa | EIVMTQSPLSLPVTPGEPASISCRSSQSLLHSN |
| | G7_CC - VL | | | GYNYLDWYLQKPGQSPQLLIYLGSNRASGVP |
| | | | | DRFSGSGSGTDFTLKISRVEAEDVGVYYCMQ |
| | | | | ALQTPFTFGCGTKVEIK |
| 734. | EpCAM_19182- | artificial | Aa | NYDMN |
| | H8_CC - | | | |
| | HCDR1 | | | |
| 735. | EpCAM_19182- | artificial | Aa | VISYDGSDKHYTDSVKG |
| | H8_CC - | | | |
| | HCDR2 | | | |
| 736. | EpCAM_19182- | artificial | Aa | RGATPVDY |
| | H8_CC - | | | |
| | HCDR3 | | | |
| 737. | EpCAM_19182- | artificial | Aa | TGTNSDVGGYNYVS |
| | H8_CC - LCDR1 | | | |
| 738. | EpCAM_19182- | artificial | Aa | DVSKRPS |
| | H8_CC - LCDR2 | | | |
| 739. | EpCAM_19182- | artificial | Aa | SSYISSSSLV |
| | H8_CC - LCDR3 | | | |
| 740. | EpCAM_19182- | artificial | Aa | QVQLVESGGGVVQPGRSLRLSCAASGFTFRN |
| | H8_CC - VH | | | YDMNWVRQAPGKCLEWVAVISYDGSDKHYT |
| | | | | DSVKGRFTISRDNSKNTLYLQMNSLRTEDTAV |
| | | | | YYCAKRGATPVDYWGQGTLVTVSS |
| 741. | EpCAM_19182- | artificial | Aa | QSALTQPASVSGSPGRSVTISCTGTNSDVGG |
| | H8_CC - VL | | | YNYVSWYQQHPGKAPKLMIYDVSKRPSGVSN |
| | | | | RFSGSKSGNTASLTISGLQAEDEADYYCSSYI |
| | | | | SSSSLVFGCGTKLTVL |
| 742. | EpCAM_19187- | artificial | Aa | NYDMN |
| | B6_N67Q_CC - | | | |
| | HCDR1 | | | |
| 743. | EpCAM_19187- | artificial | Aa | VISYDGSQKSYSDSVKG |
| | B6_N67Q_CC - | | | |
| | HCDR2 | | | |
| 744. | EpCAM_19187- | artificial | Aa | RGATPFDY |
| | B6_N67Q_CC - | | | |
| | HCDR3 | | | |
| 745. | EpCAM_19187- | artificial | Aa | GGNNIGSKSVH |
| | B6_N67Q_CC - | | | |
| | LCDR1 | | | |
| 746. | EpCAM_19187- | artificial | Aa | QDSKRPS |
| | B6_N67Q_CC - | | | |
| | LCDR2 | | | |
| 747. | EpCAM_19187- | artificial | Aa | QAWDSSTAV |
| | B6_N67Q_CC - | | | |
| | LCDR3 | | | |
| 748. | EpCAM_19187- | artificial | Aa | QVQLVESGGGVVQPGRSLRLSCAASGFTFRN |
| | B6_N67Q_CC - | | | YDMNWVRQAPGKCLEWVAVISYDGSQKSYS |
| | VH | | | DSVKGRFTISRDNSKNTLSLQMNSLRNEDTAV |
| | | | | YYCAKRGATPFDYWGQGTLVTVSS |
| 749. | EpCAM_19187- | artificial | Aa | SYELTQPPSVSVAPGQTARITCGGNNIGSKSV |
| | B6_N67Q_CC - | | | HWYQQKPGQSPVLVIYQDSKRPSGIPDRFSG |
| | VL | | | SNSGNTATLTISGTQAMDEADYYCQAWDSST |
| | | | | AVFGCGTKLTVL |
| 750. | MSLN_13203- | artificial | Aa | SNSAAWN |
| | C2_CC - | | | |
| | HCDR1 | | | |
| 751. | MSLN_13203- | artificial | Aa | RTYYRSKWYNDYAVSVKS |
| | C2_CC - | | | |
| | HCDR2 | | | |
| 752. | MSLN_13203- | artificial | Aa | AIFVVPAAMRFDY |
| | C2_CC - | | | |
| | HCDR3 | | | |
| 753. | MSLN_13203- | artificial | Aa | RSSQSLLHSNGYNYLD |
| | C2_CC - LCDR1 | | | |
| 754. | MSLN_13203- | artificial | Aa | LGSNRAS |
| | C2_CC - LCDR2 | | | |
| 755. | MSLN_13203- | artificial | Aa | MQALQTPT |
| | C2_CC - LCDR3 | | | |
| 756. | MSLN_13203- | artificial | Aa | QVQLQQSGPGLVKPSQTLSLTCAISGDSVSS |
| | C2_CC - VH | | | NSAAWNWIRQSPSRCLEWLGRTYYRSKWYN |
| | | | | DYAVSVKSRITINPDISKNQFSLQLNSVTPEDT |
| | | | | AVYYCARAIFVVPAAMRFDYWGQGTLVTVSS |
| 757. | MSLN_13203- | artificial | Aa | EIVMTQSPLSLPVTPGEPASISCRSSQSLLHSN |
| | C2_CC - VL | | | GYNYLDWYLQKPGQSPQLLIYLGSNRASGVP |
| | | | | DRFSGSGSGTDFTLKISRVEAEDVGVYYCMQ |
| | | | | ALQTPTFGCGTKVDIK |
| 758. | MSLN_13203- | artificial | Aa | SNYMS |
| | F11_CC - | | | |
| | HCDR1 | | | |
| 759. | MSLN_13203- | artificial | Aa | VIYSSGNTYYADSVKG |
| | F11_CC - | | | |
| | HCDR2 | | | |
| 760. | MSLN_13203- | artificial | Aa | GSYYAFDI |
| | F11_CC - | | | |
| | HCDR3 | | | |
| 761. | MSLN_13203- | artificial | Aa | GLSSGSVSTTYYPS |
| | F11_CC - | | | |
| | LCDR1 | | | |
| 762. | MSLN_13203- | artificial | Aa | STNTRSS |
| | F11_CC - | | | |
| | LCDR2 | | | |
| 763. | MSLN_13203- | artificial | Aa | VLYMGSGIWV |
| | F11_CC - | | | |
| | LCDR3 | | | |
| 764. | MSLN_13203- | artificial | Aa | EVQLVESGGGLIQPGGSLRLSCAVSGFTVSS |
| | F11_CC - VH | | | NYMSWVRQAPGKCLEWVSVIYSSGNTYYADS |
| | | | | VKGRFTISRDNSKNTLYLQMNSLRAEDTAVYY |
| | | | | CASGSYYAFDIWGQGTMVTVSS |
| 765. | MSLN_13203- | artificial | Aa | QTVVTQEPSLTVSPGGTVTLTCGLSSGSVSTT |
| | F11_CC - VL | | | YYPSWYQQTPGQAPRTLIYSTNTRSSGVPDR |
| | | | | FSGSILGNKAALTITGAQADDESDYYCVLYMG |
| | | | | SGIWVFGCGTKLTVL |
| 766. | MSLN_13204- | artificial | Aa | NAWMS |
| | A9_CC - HCDR1 | | | |
| 767. | MSLN_13204- | artificial | Aa | RIKTKTDGGTTDYAAPVKG |
| | A9_CC - HCDR2 | | | |
| 768. | MSLN_13204- | artificial | Aa | DFRIMGATWFDP |
| | A9_CC - HCDR3 | | | |
| 769. | MSLN_13204- | artificial | Aa | SGDKLGDKYAS |
| | A9_CC - LCDR1 | | | |
| 770. | MSLN_13204- | artificial | Aa | QHSRRPS |
| | A9_CC - LCDR2 | | | |
| 771. | MSLN_13204- | artificial | Aa | QAWDSSTVV |
| | A9_CC - LCDR3 | | | |
| 772. | MSLN_13204- | artificial | Aa | EVQLVESGGGLVKPGGSLRLSCAASGFTFSN |
| | A9_CC - VH | | | AWMSWVRQAPGKCLEWVGRIKTKTDGGTTD |
| | | | | YAAPVKGRFTISRDDSKNTLYLQMNSLKTEDT |
| | | | | AVYYCTTDFRIMGATWFDPWGQGTLVTVSS |
| 773. | MSLN_13204- | artificial | Aa | SYELTQPPSVSVSPGQTASITCSGDKLGDKYA |
| | A9_CC - VL | | | SWYQQKPGQSPVLVIYQHSRRPSGIPERFSG |
| | | | | SNSGNTATLTISGTQAMDEADYYCQAWDSST |
| | | | | VVFGCGTKLTVL |
| 774. | MSLN_13204- | artificial | Aa | SYSMN |
| | D11_CC - | | | |
| | HCDR1 | | | |
| 775. | MSLN_13204- | artificial | Aa | SISSRSSYIHYADSVKG |
| | D11_CC - | | | |
| | HCDR2 | | | |
| 776. | MSLN_13204- | artificial | Aa | VQRAGLDY |
| | D11_CC - | | | |
| | HCDR3 | | | |
| 777. | MSLN_13204- | artificial | Aa | TGSSSDVGNYNLVS |
| | D11_CC - | | | |
| | LCDR1 | | | |
| 778. | MSLN_13204- | artificial | Aa | EVSNRPS |
| | D11_CC - | | | |
| | LCDR2 | | | |
| 779. | MSLN_13204- | artificial | Aa | SSYTSSSTWV |
| | D11_CC - | | | |
| | LCDR3 | | | |
| 780. | MSLN_13204- | artificial | Aa | EVQLVESGGGLVKPGGSLRLSCAASGFTFSS |
| | D11_CC - VH | | | YSMNWVRQAPGKCLEWVSSISSRSSYIHYAD |
| | | | | SVKGRFTISRDNAKNSLNLQMNSLRAEDTAVY |
| | | | | YCARVQRAGLDYWGQGTLVTVSS |
| 781. | MSLN_13204- | artificial | Aa | QSALTQPASVSGSPGQSITISCTGSSSDVGNY |
| | D11_CC - VL | | | NLVSWYQQHPGKAPKLMISEVSNRPSGVSDR |
| | | | | FSGSKSGNTASLTISGLQAEDEADYYCSSYTS |
| | | | | SSTWVFGCGTKLTVL |
| 782. | MSLN_13204- | artificial | Aa | SSSYYWG |
| | F11_CC - | | | |
| | HCDR1 | | | |
| 783. | MSLN_13204- | artificial | Aa | SIYYSGSTNYNPSLKS |
| | F11_CC - | | | |
| | HCDR2 | | | |
| 784. | MSLN_13204- | artificial | Aa | PSNYDAFDI |
| | F11_CC - | | | |
| | HCDR3 | | | |
| 785. | MSLN_13204- | artificial | Aa | TGSSSNIGAGYDVH |
| | F11_CC - | | | |
| | LCDR1 | | | |
| 786. | MSLN_13204- | artificial | Aa | GNSNRPS |
| | F11_CC - | | | |
| | LCDR2 | | | |
| 787. | MSLN_13204- | artificial | Aa | QSYDSSLGGWV |
| | F11_CC - | | | |
| | LCDR3 | | | |
| 788. | MSLN_13204- | artificial | Aa | QVQLQESGPGLVKPSETLSLTCTVSGGSLSS |
| | F11_CC - VH | | | SSYYWGWIRQPPGKCLEWIGSIYYSGSTNYN |
| | | | | PSLKSRVTISADTSKNQFSLKLSSVTAADTAVY |
| | | | | YCARPSNYDAFDIWGQGTMVTVSS |
| 789. | MSLN_13204- | artificial | Aa | QSVLTQPPSVSGAPGQRVTISCTGSSSNIGAG |
| | F11_CC - VL | | | YDVHWYQQLPGTAPKLLIYGNSNRPSGVPDR |
| | | | | FSGSKSGTSASLAITGLQAEDEADYYCQSYDS |
| | | | | SLGGWVFGCGTKLTVL |
| 790. | MSLN_13204- | artificial | Aa | SGGFFWS |
| | H6_CC - | | | |
| | HCDR1 | | | |
| 791. | MSLN_13204- | artificial | Aa | YIYYSGSTYYNPSLRS |
| | H6_CC - | | | |
| | HCDR2 | | | |
| 792. | MSLN_13204- | artificial | Aa | DPGSYRVWFDP |
| | H6_CC - | | | |
| | HCDR3 | | | |
| 793. | MSLN_13204- | artificial | Aa | RASQNIKNYLN |
| | H6_CC - LCDR1 | | | |
| 794. | MSLN_13204- | artificial | Aa | DASSLQS |
| | H6_CC - LCDR2 | | | |
| 795. | MSLN_13204- | artificial | Aa | QQSYSTPFT |
| | H6_CC - LCDR3 | | | |
| 796. | MSLN_13204- | artificial | Aa | QVQLQESGPGLVKPSQTLSLTCTVSGGSISSG |
| | H6_CC - VH | | | GFFWSWIRQHPGKCLEWIGYIYYSGSTYYNP |
| | | | | SLRSRVTISVDTSKNQFSLKLSSVTAADTAVYY |
| | | | | CARDPGSYRVWFDPWGQGTLVTVSS |
| 797. | MSLN_13204- | artificial | Aa | EIQMTQSPSSLSASVGDRVTITCRASQNIKNYL |
| | H6_CC - VL | | | NWYQQKPGRAPKLLIYDASSLQSGDPSRFSG |
| | | | | SGSGTDFTLTISSLQPEDFATYYCQQSYSTPF |
| | | | | TFGCGTKVEIK |
| 798. | MSLN_13213- | artificial | Aa | DHYMS |
| | A9_CC - HCDR1 | | | |
| 799. | MSLN_13213- | artificial | Aa | YISNSGSIIYYVDSVKG |
| | A9_CC - HCDR2 | | | |
| 800. | MSLN_13213- | artificial | Aa | DVRTAFDY |
| | A9_CC - HCDR3 | | | |
| 801. | MSLN_13213- | artificial | Aa | RASQSIGSWLA |
| | A9_CC - LCDR1 | | | |
| 802. | MSLN_13213- | artificial | Aa | AASSLQS |
| | A9_CC - LCDR2 | | | |
| 803. | MSLN_13213- | artificial | Aa | QQANSFPPT |
| | A9_CC - LCDR3 | | | |
| 804. | MSLN_13213- | artificial | Aa | QVQLVESGGGLVKPGGSLRLSCAASGFTFSD |
| | A9_CC - VH | | | HYMSWIRQAPGKCLEWISYISNSGSIIYYVDSV |
| | | | | KGRFTISRDNAKNSLYLQMNSLRAEDTAVYYC |
| | | | | ARDVRTAFDYWGQGTLVTVSS |
| 805. | MSLN_13213- | artificial | Aa | EIQMTQSPSSVSASVGDRVTITCRASQSIGSW |
| | A9_CC - VL | | | LAWYQQKPGKAPNLLIYAASSLQSGVPSRFS |
| | | | | GSGSGTDFTLTISSLQPEDFATYYCQQANSFP |
| | | | | PTFGCGTKVEIK |
| 806. | MSLN_13215- | artificial | Aa | SSSYFWG |
| | B12_CC - | | | |
| | HCDR1 | | | |
| 807. | MSLN_13215- | artificial | Aa | NIYYSGSSNYNPSLKS |
| | B12_CC - | | | |
| | HCDR2 | | | |
| 808. | MSLN_13215- | artificial | Aa | LPRGDRDAFDI |
| | B12_CC - | | | |
| | HCDR3 | | | |
| 809. | MSLN_13215- | artificial | Aa | RASQGISNYLA |
| | B12_CC - | | | |
| | LCDR1 | | | |
| 810. | MSLN_13215- | artificial | Aa | AASTLQS |
| | B12 CC - | | | |
| | LCDR2 | | | |
| 811. | MSLN_13215- | artificial | Aa | QQSYSTPFT |
| | B12_CC - | | | |
| | LCDR3 | | | |
| 812. | MSLN_13215- | artificial | Aa | QVQLQESGPGLVKPSETLSLTCTVSGGSISSS |
| | B12_CC - VH | | | SYFWGWIRQPPGKCLEWIGNIYYSGSSNYNP |
| | | | | SLKSRVTISVDTSKNQFSLKLSSVTAADTAVYY |
| | | | | CARLPRGDRDAFDIWGQGTMVTVSS |
| 813. | MSLN_13215- | artificial | Aa | EIVMTQSPSSLSASVGDRVTITCRASQGISNYL |
| | B12_CC - VL | | | AWYQQKPGKVPKLLIYAASTLQSGVPSRFSG |
| | | | | SGSGTDFTLTISSLQPEDFATYYCQQSYSTPF |
| | | | | TFGCGTKVEIK |
| 814. | MSLN_13215- | artificial | Aa | SSSYFWG |
| | B12_LC_V3Q_C | | | |
| | C - HCDR1 | | | |
| 815. | MSLN_13215- | artificial | Aa | NIYYSGSSNYNPSLKS |
| | B12_LC_V3Q_C | | | |
| | C - HCDR2 | | | |
| 816. | MSLN_13215- | artificial | Aa | LPRGDRDAFDI |
| | B12_LC_V3Q_C | | | |
| | C - HCDR3 | | | |
| 817. | MSLN_13215- | artificial | Aa | RASQGISNYLA |
| | B12_LC_V3Q_C | | | |
| | C - LCDR1 | | | |
| 818. | MSLN_13215- | artificial | Aa | AASTLQS |
| | B12_LC_V3Q_C | | | |
| | C - LCDR2 | | | |
| 819. | MSLN_13215- | artificial | Aa | QQSYSTPFT |
| | B12_LC_V3Q_C | | | |
| | C - LCDR3 | | | |
| 820. | MSLN_13215- | artificial | Aa | QVQLQESGPGLVKPSETLSLTCTVSGGSISSS |
| | B12_LC_V3Q_C | | | SYFWGWIRQPPGKCLEWIGNIYYSGSSNYNP |
| | C - VH | | | SLKSRVTISVDTSKNQFSLKLSSVTAADTAVYY |
| | | | | CARLPRGDRDAFDIWGQGTMVTVSS |
| 821. | MSLN_13215- | artificial | Aa | EIQMTQSPSSLSASVGDRVTITCRASQGISNYL |
| | B12_LC_V3Q_C | | | AWYQQKPGKVPKLLIYAASTLQSGVPSRFSG |
| | C - VL | | | SGSGTDFTLTISSLQPEDFATYYCQQSYSTPF |
| | | | | TFGCGTKVEIK |
| 822. | MSLN_13216- | artificial | Aa | SGGHFWS |
| | B12_CC - | | | |
| | HCDR1 | | | |
| 823. | MSLN_13216- | artificial | Aa | YIYYSGSTYSTPSLTS |
| | B12_CC - | | | |
| | HCDR2 | | | |
| 824. | MSLN_13216- | artificial | Aa | EGQSGSFDI |
| | B12_CC - | | | |
| | HCDR3 | | | |
| 825. | MSLN_13216- | artificial | Aa | TGTSSDVGGSDYVS |
| | B12_CC - | | | |
| | LCDR1 | | | |
| 826. | MSLN_13216- | artificial | Aa | EVSNRPS |
| | B12_CC - | | | |
| | LCDR2 | | | |
| 827. | MSLN_13216- | artificial | Aa | SSYTTTGTLV |
| | B12_CC - | | | |
| | LCDR3 | | | |
| 828. | MSLN_13216- | artificial | Aa | QVQLQESGPGLVKPSQTLSLTCTVSGGSISSG |
| | B12_CC - VH | | | GHFWSWIRQHPGKCLEWIGYIYYSGSTYSTP |
| | | | | SLTSRVTMSRDTSKNQFSLKLSSVTAADTAVY |
| | | | | YCAREGQSGSFDIWGQGTMVTVSS |
| 829. | MSLN_13216- | artificial | Aa | QSALTQPASVSGSPGQSITISCTGTSSDVGGS |
| | B12_CC - VL | | | DYVSWYRQHPGKAPKLIIYEVSNRPSGVSNRF |
| | | | | SGSKSGNTASLTISGLQAEDEADYYCSSYTTT |
| | | | | GTLVFGCGTKLTVL |
| 830. | MSLN_13216- | artificial | Aa | SYGMH |
| | B4_CC - HCDR1 | | | |
| 831. | MSLN_13216- | artificial | Aa | VIWKDGNNKYYADSVKG |
| | B4_CC - HCDR2 | | | |
| 832. | MSLN_13216- | artificial | Aa | GLNYYYGMDV |
| | B4_CC - HCDR3 | | | |
| 833. | MSLN_13216- | artificial | Aa | TRSNGGIANNYVQ |
| | B4_CC - LCDR1 | | | |
| 834. | MSLN_13216- | artificial | Aa | ENNQRPS |
| | B4_CC - LCDR2 | | | |
| 835. | MSLN_13216- | artificial | Aa | QSYDGSHHVV |
| | B4_CC - LCDR3 | | | |
| 836. | MSLN_13216- | artificial | Aa | QVQLVESGGGVVQPGRSLRLSCAASGFTFSS |
| | B4_CC - VH | | | YGMHWVRQAPGKCLEWVAVIWKDGNNKYYA |
| | | | | DSVKGRFTISRDNSKNTLYLQMNSLRAEDTAV |
| | | | | YYCARGLNYYYGMDVWGQGTTVTVSS |
| 837. | MSLN_13216- | artificial | Aa | NFMLTQPHSVSESPGKTATISCTRSNGGIANN |
| | B4_CC - VL | | | YVQWYQQRPGSSPTIVIYENNQRPSGVPDRF |
| | | | | SGSIDSSSNSASLTISGLKTEDEADYYCQSYD |
| | | | | GSHHVVFGCGTKLTVL |
| 838. | MSLN_13216- | artificial | Aa | GYYIH |
| | C1_CC - | | | |
| | HCDR1 | | | |
| 839. | MSLN_13216- | artificial | Aa | WINPKSGGTHYAQKFQG |
| | C1_CC - | | | |
| | HCDR2 | | | |
| 840. | MSLN_13216- | artificial | Aa | AEARLAARQEYYYFYGMDV |
| | C1_CC - | | | |
| | HCDR3 | | | |
| 841. | MSLN_13216- | artificial | Aa | SGDKLGDKYAS |
| | C1_CC - LCDR1 | | | |
| 842. | MSLN_13216- | artificial | Aa | QDSKRPS |
| | C1_CC - LCDR2 | | | |
| 843. | MSLN_13216- | artificial | Aa | QAWDSSTVV |
| | C1_CC - LCDR3 | | | |
| 844. | MSLN_13216- | artificial | Aa | QVQLVQSGAEVKKPGASVKVSCKASGYTFTG |
| | C1_CC - VH | | | YYIHWVRQAPGQCLEWMGWINPKSGGTHYA |
| | | | | QKFQGRVTMTRDTSISTAYMELSRLRSDDTAV |
| | | | | YYCARAEARLAARQEYYYFYGMDVWGQGTT |
| | | | | VTVSS |
| 845. | MSLN_13216- | artificial | Aa | SYELTQPASVSVSPGQTASITCSGDKLGDKYA |
| | C1_CC - VL | | | SWYQQKPGQSPVLVIYQDSKRPSGIPERFSG |
| | | | | SNSGNTATLTISGTQAMDEADYYCQAWDSST |
| | | | | VVFGCGTKLTVL |
| 846. | MSLN_13229- | artificial | Aa | SGAYFWS |
| | C9_CC - | | | |
| | HCDR1 | | | |
| 847. | MSLN_13229- | artificial | Aa | YIYYSGSTYTNPSLRD |
| | C9_CC - | | | |
| | HCDR2 | | | |
| 848. | MSLN_13229- | artificial | Aa | EGAGYVFDI |
| | C9_CC - | | | |
| | HCDR3 | | | |
| 849. | MSLN_13229- | artificial | Aa | TGTSSDVGGYNYVS |
| | C9_CC - LCDR1 | | | |
| 850. | MSLN_13229- | artificial | Aa | EVSNRPS |
| | C9_CC - LCDR2 | | | |
| 851. | MSLN_13229- | artificial | Aa | QVWDSSSDHVV |
| | C9_CC - LCDR3 | | | |
| 852. | MSLN_13229- | artificial | Aa | QVQLQESGPGLVKPSQTLSLTCTVSGGSISSG |
| | C9_CC - VH | | | AYFWSWIRQHPGKCLEWIGYIYYSGSTYTNPS |
| | | | | LRDRLKISVDTSKNQFSLKLSSVTAADTAMYY |
| | | | | CAREGAGYVFDIWGQGTMVTVSS |
| 853. | MSLN_13229- | artificial | Aa | QSALTQPASVSGSPGQSITISCTGTSSDVGGY |
| | C9_CC - VL | | | NYVSWYQQHPGKAPKLMIYEVSNRPSGVSNR |
| | | | | FSGSKSGNTASLTISGLQAGDEADYFCQVWD |
| | | | | SSSDHVVFGCGTKLTVL |
| 854. | MSLN_13238- | artificial | Aa | SGGYYWN |
| | G11_CC - | | | |
| | HCDR1 | | | |
| 855. | MSLN_13238- | artificial | Aa | YIFYSGITYSNPSLKS |
| | G11_CC - | | | |
| | HCDR2 | | | |
| 856. | MSLN_13238- | artificial | Aa | GLVRGAPDAFDI |
| | G11_CC - | | | |
| | HCDR3 | | | |
| 857. | MSLN_13238- | artificial | Aa | QASQDISNYLN |
| | G11_CC - | | | |
| | LCDR1 | | | |
| 858. | MSLN_13238- | artificial | Aa | AASSLQG |
| | G11_CC - | | | |
| | LCDR2 | | | |
| 859. | MSLN_13238- | artificial | Aa | QQSYSTPFT |
| | G11_CC - | | | |
| | LCDR3 | | | |
| 860. | MSLN_13238- | artificial | Aa | QVQLQESGPGLVKPSQTLSLTCTVSGGSISSG |
| | G11_CC - VH | | | GYYWNWIRQHPGQCLEWIGYIFYSGITYSNPS |
| | | | | LKSLFTISLDTSKNQFSLKLSSVTAADTAVYYC |
| | | | | ARGLVRGAPDAFDIWGQGTMVTVSS |
| 861. | MSLN_13238- | artificial | Aa | EIQMTQSPSSLSASVGDRVTITCQASQDISNYL |
| | G11_CC - VL | | | NWYQLKPGKAPKLLIQAASSLQGGVPSRFSG |
| | | | | SGSGTDFTLTISSLQPEDFATYYCQQSYSTPF |
| | | | | TFGCGTKVEIK |
| 862. | MSLN_13239- | artificial | Aa | SYYWS |
| | D5_CC - | | | |
| | HCDR1 | | | |
| 863. | MSLN_13239- | artificial | Aa | RIYYNGNTYYNPSLKS |
| | D5_CC - | | | |
| | HCDR2 | | | |
| 864. | MSLN_13239- | artificial | Aa | PKLGIDAFDI |
| | D5_CC - | | | |
| | HCDR3 | | | |
| 865. | MSLN_13239- | artificial | Aa | TGSSSNIGAGYDVH |
| | D5_CC - LCDR1 | | | |
| 866. | MSLN_13239- | artificial | Aa | GNSNRPS |
| | D5_CC - LCDR2 | | | |
| 867. | MSLN_13239- | artificial | Aa | QSHDSSLSGSV |
| | D5_CC - LCDR3 | | | |
| 868. | MSLN_13239- | artificial | Aa | QVQLQESGPGLVKPSETLSLTCTVSGGSISSY |
| | D5_CC - VH | | | YWSWIRQPPGKCLEWIGRIYYNGNTYYNPSL |
| | | | | KSRVTISGDTSKNQFSLKLSSVTAADTAVYYC |
| | | | | ARPKLGIDAFDIWGQGTMVTVSS |
| 869. | MSLN_13239- | artificial | Aa | QSVLTQPPSVSGAPGQRVTISCTGSSSNIGAG |
| | D5_CC - VL | | | YDVHWYQKLPGTAPKLLIYGNSNRPSGVPDR |
| | | | | FSGSKSGTSASLAITGLQAEDEADYYCQSHDS |
| | | | | SLSGSVFGCGTKLTVL |
| 870. | MSLN_13254- | artificial | Aa | SGGYFWS |
| | B10_CC - | | | |
| | HCDR1 | | | |
| 871. | MSLN_13254- | artificial | Aa | YIYYSGSTYTNPSLRD |
| | B10_CC - | | | |
| | HCDR2 | | | |
| 872. | MSLN_13254- | artificial | Aa | EGAGYAFDI |
| | B10_CC - | | | |
| | HCDR3 | | | |
| 873. | MSLN_13254- | artificial | Aa | TGTSSDVGGYNYVS |
| | B10_CC - | | | |
| | LCDR1 | | | |
| 874. | MSLN_13254- | artificial | Aa | EVSNRPS |
| | B10_CC - | | | |
| | LCDR2 | | | |
| 875. | MSLN_13254- | artificial | Aa | SSYTSSSTLV |
| | B10_CC - | | | |
| | LCDR3 | | | |
| 876. | MSLN_13254- | artificial | Aa | QVQLQESGGGLVKPSETLSLTCTVSGGSISSG |
| | B10_CC - VH | | | GYFWSWIRQHPGKCLEWIGYIYYSGSTYTNP |
| | | | | SLRDRLKISVDTSKNQFSLKLSSVTAADTAMY |
| | | | | YCAREGAGYAFDIWGQGTMVTVSS |
| 877. | MSLN_13254- | artificial | Aa | QSALTQPASVSGSPGQSITISCTGTSSDVGGY |
| | B10_CC - VL | | | NYVSWYQQHPGKAPKLMIYEVSNRPSGVSNR |
| | | | | FSGSKSGNTASLTISGLQAEDEADYYCSSYTS |
| | | | | SSTLVFGCGTKLTVL |
| 878. | MSLN_13256- | artificial | Aa | SYGMH |
| | H4_CC - | | | |
| | HCDR1 | | | |
| 879. | MSLN_13256- | artificial | Aa | VISYDGSNKYYADSVKG |
| | H4_CC - | | | |
| | HCDR2 | | | |
| 880. | MSLN_13256- | artificial | Aa | EGAYFGSGSYYPLYYYYAMDV |
| | H4_CC - | | | |
| | HCDR3 | | | |
| 881. | MSLN_13256- | artificial | Aa | RASQSVSSSYLA |
| | H4_CC - LCDR1 | | | |
| 882. | MSLN_13256- | artificial | Aa | GASIRAT |
| | H4_CC - LCDR2 | | | |
| 883. | MSLN_13256- | artificial | Aa | QQYGSSLFT |
| | H4_CC - LCDR3 | | | |
| 884. | MSLN_13256- | artificial | Aa | QVQLVESGGGVVQPGRSLRLSCAASGFTFSS |
| | H4_CC - VH | | | YGMHWVRQAPGKCLEWVAVISYDGSNKYYA |
| | | | | DSVKGRFTISRDNSKNTLYLQMNSLRAEDTAV |
| | | | | YYCAREGAYFGSGSYYPLYYYYAMDVWGQG |
| | | | | TTVTVSS |
| 885. | MSLN_13256- | artificial | Aa | EIVMTQSPGTLSLSPGERATLSCRASQSVSSS |
| | H4_CC - VL | | | YLAWYQQKPGQAPRLLIYGASIRATGIPDRFS |
| | | | | GSGSGTDFTLTISRLEPEDFAVYYCQQYGSSL |
| | | | | FTFGCGTRLEIK |
| 886. | MSLN_13266- | artificial | Aa | DHYMS |
| | C1_CC - | | | |
| | HCDR1 | | | |
| 887. | MSLN_13266- | artificial | Aa | YISSSGSTIYYVDSVKG |
| | C1_CC - | | | |
| | HCDR2 | | | |
| 888. | MSLN_13266- | artificial | Aa | DVRTAFDY |
| | C1_CC - | | | |
| | HCDR3 | | | |
| 889. | MSLN_13266- | artificial | Aa | RASQGISSWLA |
| | C1_CC - LCDR1 | | | |
| 890. | MSLN_13266- | artificial | Aa | AASGLQS |
| | C1_CC - LCDR2 | | | |
| 891. | MSLN_13266- | artificial | Aa | QQANSFPPT |
| | C1_CC - LCDR3 | | | |
| 892. | MSLN_13266- | artificial | Aa | QVQLVESGGGLVKPGGSLRLSCAASGFTFSD |
| | C1_CC - VH | | | HYMSWIRQTPGKCLEWVSYISSSGSTIYYVDS |
| | | | | VKGRFTISRDNAKNSLYLQMNSLRAEDTAVYY |
| | | | | CARDVRTAFDYWGQGTLVTVSS |
| 893. | MSLN_13266- | artificial | Aa | EIQMTQSPSSVSASVGDRVTITCRASQGISSW |
| | C1_CC - VL | | | LAWYQQKPGKAPKLLIYAASGLQSGVPSRFS |
| | | | | GSGSGTDFTLTISSLQPEDFATYYCQQANSFP |
| | | | | PTFGCGTKVEIK |
| 894. | MSLN_13268- | artificial | Aa | SYTMS |
| | A4_CC - HCDR1 | | | |
| 895. | MSLN_13268- | artificial | Aa | AISGSGGNTYYADSVKG |
| | A4_CC - HCDR2 | | | |
| 896. | MSLN_13268- | artificial | Aa | VGRAALDY |
| | A4_CC - HCDR3 | | | |
| 897. | MSLN_13268- | artificial | Aa | TGTSSDVGSYNLVS |
| | A4_CC - LCDR1 | | | |
| 898. | MSLN_13268- | artificial | Aa | EVSKRPS |
| | A4_CC - LCDR2 | | | |
| 899. | MSLN_13268- | artificial | Aa | SSYTSSSTVV |
| | A4_CC - LCDR3 | | | |
| 900. | MSLN_13268- | artificial | Aa | EVQLLESGGGLVQPGGSPRLSCAVSGFTFSS |
| | A4_CC - VH | | | YTMSWVRQAPGKCLEWVSAISGSGGNTYYA |
| | | | | DSVKGRSTISRDNSRNTLYLQMNSLRAEDTAV |
| | | | | YYCAKVGRAALDYWGQGTLVTVSS |
| 901. | MSLN_13268- | artificial | Aa | QSALTQPPSVSGSPGQSITISCTGTSSDVGSY |
| | A4_CC - VL | | | NLVSWYQQHPGKAPKLMIYEVSKRPSGVSNR |
| | | | | FSGSKSGNTASLTISGLQAEDEADYYCSSYTS |
| | | | | SSTVVFGCGTKLTVL |
| 902. | MSLN_13269- | artificial | Aa | SYAMS |
| | A6_CC - HCDR1 | | | |
| 903. | MSLN_13269- | artificial | Aa | AISGSGGSTYYADSVKG |
| | A6_CC - HCDR2 | | | |
| 904. | MSLN_13269- | artificial | Aa | EGYYDSSGYPLYYYFGMDV |
| | A6_CC - HCDR3 | | | |
| 905. | MSLN_13269- | artificial | Aa | RASQSVSSSYLA |
| | A6_CC - LCDR1 | | | |
| 906. | MSLN_13269- | artificial | Aa | GASSRAT |
| | A6_CC - LCDR2 | | | |
| 907. | MSLN_13269- | artificial | Aa | QRYGSSPIFT |
| | A6_CC - LCDR3 | | | |
| 908. | MSLN_13269- | artificial | Aa | EVQLLESGGGLVQPGGSLRLSCAASGFTFSS |
| | A6_CC - VH | | | YAMSWVRQAPGKCLEWVSAISGSGGSTYYA |
| | | | | DSVKGRFTISRDNSKNTLYLQMNSLRAEDTAV |
| | | | | YYCAREGYYDSSGYPLYYYFGMDVWGQGTT |
| | | | | VTVSS |
| 909. | MSLN_13269- | artificial | Aa | EIVLTQSPGTLSLSPGERATLSCRASQSVSSS |
| | A6_CC - VL | | | YLAWYQQKPGQAPRLLIYGASSRATGIPDRFS |
| | | | | GSGSGTDFTLTISRLEPEDFAVYYCQRYGSSP |
| | | | | IFTFGCGTKVEIK |
| 910. | MSLN_13270- | artificial | Aa | NAWMS |
| | A3_CC - HCDR1 | | | |
| 911. | MSLN_13270- | artificial | Aa | RIKTKTDGGTTDYAAPVKG |
| | A3_CC - HCDR2 | | | |
| 912. | MSLN_13270- | artificial | Aa | DFRIMGATWFDP |
| | A3_CC - HCDR3 | | | |
| 913. | MSLN_13270- | artificial | Aa | SGSSSNIGSYSVN |
| | A3_CC - LCDR1 | | | |
| 914. | MSLN_13270- | artificial | Aa | SNNQRPS |
| | A3_CC - LCDR2 | | | |
| 915. | MSLN_13270- | artificial | Aa | AAWDDSLSGRGVA |
| | A3_CC - LCDR3 | | | |
| 916. | MSLN_13270- | artificial | Aa | EVQLVESGGGLVKPGGSLRLSCAASGFTFSN |
| | A3_CC - VH | | | AWMSWVRQAPGKCLEWVGRIKTKTDGGTTD |
| | | | | YAAPVKGRFTISRDDSKNTLYLQMNSLKTEDT |
| | | | | AVYYCTTDFRIMGATWFDPWGQGTLVTVSS |
| 917. | MSLN_13270- | artificial | Aa | QSVLTQPSSASGTPGQRVTISCSGSSSNIGSY |
| | A3_CC - VL | | | SVNWYQQLPGTAPKLLIYSNNQRPSGVPDRF |
| | | | | SGSKSGTSASLAISGLRSEDEADYYCAAWDD |
| | | | | SLSGRGVAFGCGTKLTVL |
| 918. | MSLN_13317- | artificial | Aa | DYYMS |
| | C7_CC - | | | |
| | HCDR1 | | | |
| 919. | MSLN_13317- | artificial | Aa | YISSSGSMIYYIDSVKG |
| | C7_CC - | | | |
| | HCDR2 | | | |
| 920. | MSLN_13317- | artificial | Aa | DLGPSFDY |
| | C7_CC - | | | |
| | HCDR3 | | | |
| 921. | MSLN_13317- | artificial | Aa | RASQGIGSWLA |
| | C7_CC - LCDR1 | | | |
| 922. | MSLN_13317- | artificial | Aa | GASGLQS |
| | C7_CC - LCDR2 | | | |
| 923. | MSLN_13317- | artificial | Aa | QQANSFPRT |
| | C7_CC - LCDR3 | | | |
| 924. | MSLN_13317- | artificial | Aa | QVQLVESGGGLVKPGGSLRLSCAASGFTFSD |
| | C7_CC - VH | | | YYMSWIRQAPGKCLEWISYISSSGSMIYYIDSV |
| | | | | KGRFTISRDNAKNSLYLQMNSLRAEDTAVYYC |
| | | | | ARDLGPSFDYWGQGSLVTVSS |
| 925. | MSLN_13317- | artificial | Aa | EIQMTQSPSSVAATVGDRVTITCRASQGIGSW |
| | C7_CC - VL | | | LAWYQQKPGKAPKLLIYGASGLQSGVPSRFS |
| | | | | GSGSGTDFTLTISSLQPEDFATYYCQQANSFP |
| | | | | RTFGCGTKVEIK |
| 926. | MSLN_13317- | artificial | Aa | DHYMS |
| | F9_CC - HCDR1 | | | |
| 927. | MSLN_3317- | artificial | Aa | YISNSGSTIYYADSVKG |
| | F9_CC - HCDR2 | | | |
| 928. | MSLN_13317- | artificial | Aa | DQRNAFDI |
| | F9_CC - HCDR3 | | | |
| 929. | MSLN_13317- | artificial | Aa | RASQGIGSWLA |
| | F9_CC - LCDR1 | | | |
| 930. | MSLN_13317- | artificial | Aa | AASGLQS |
| | F9_CC - LCDR2 | | | |
| 931. | MSLN_13317- | artificial | Aa | QQSYSNPLT |
| | F9_CC - LCDR3 | | | |
| 932. | MSLN_13317- | artificial | Aa | QVQLVESGGGLVKPGGSLRLSCAASGFTFSD |
| | F9_CC - VH | | | HYMSWIRQAPGKCLEWISYISNSGSTIYYADS |
| | | | | VKGRFTISRDNAKNSLYLQMNSLRAEDTAVYY |
| | | | | CARDQRNAFDIWGQGTMVTVSS |
| 933. | MSLN_13317- | artificial | Aa | AIQMTQSPSSLSASVGDRVTITCRASQGIGSW |
| | F9_CC - VL | | | LAWYQQKPGKAPKLLIYAASGLQSGVPSRFS |
| | | | | GSGSGTDFTLTISSLQPEDFATYYCQQSYSNP |
| | | | | LTFGCGTKVEIK |
| 934. | MSLN_13318- | artificial | Aa | SSSYYWG |
| | B9_CC - HCDR1 | | | |
| 935. | MSLN_13318- | artificial | Aa | SIYYSGTTRYNPSLRS |
| | B9_CC - HCDR2 | | | |
| 936. | MSLN_13318- | artificial | Aa | PGAGHDGFDI |
| | B9_CC - HCDR3 | | | |
| 937. | MSLN_13318- | artificial | Aa | SGSSSNIGSNYVY |
| | B9_CC - LCDR1 | | | |
| 938. | MSLN_13318- | artificial | Aa | DNNKRPS |
| | B9_CC - LCDR2 | | | |
| 939. | MSLN_13318- | artificial | Aa | AAWDDSLSGWV |
| | B9_CC - LCDR3 | | | |
| 940. | MSLN_13318- | artificial | Aa | QVQLQESGPGLLKPSETLSLTCTVSGGSISSS |
| | B9_CC - VH | | | SYYWGWIRQPPGKCLEWIGSIYYSGTTRYNP |
| | | | | SLRSRVTTSLDASKNRLSLQLSSVTAADTAVY |
| | | | | YCARPGAGHDGFDIWGQGTMVTVSS |
| 941. | MSLN_13318- | artificial | Aa | QSVLTQPPSASGTPGQRVTISCSGSSSNIGSN |
| | B9_CC - VL | | | YVYWYQQLPGTAPKLLIYDNNKRPSGIPDRFS |
| | | | | GSKSGTSASLAISGLRSEDEADYYCAAWDDS |
| | | | | LSGWVFGCGTKLTVL |
| 942. | MSLN_13319- | artificial | Aa | SYYWS |
| | B8_CC - HCDR1 | | | |
| 943. | MSLN_13319- | artificial | Aa | RIYSSGSANYNPSLKS |
| | B8_CC - HCDR2 | | | |
| 944. | MSLN_13319- | artificial | Aa | EGQWRVPAQYYYFGMDV |
| | B8_CC - HCDR3 | | | |
| 945. | MSLN_13319- | artificial | Aa | RASQSVSSSYLA |
| | B8_CC - LCDR1 | | | |
| 946. | MSLN_13319- | artificial | Aa | GASSRAT |
| | B8_CC - LCDR2 | | | |
| 947. | MSLN_13319- | artificial | Aa | QQYGSSIT |
| | B8_CC - LCDR3 | | | |
| 948. | MSLN_13319- | artificial | Aa | QVQLQESGPGLVKPSETLSLTCTVSGGSISSY |
| | B8_CC - VH | | | YWSWIRQPAGKCLEWIGRIYSSGSANYNPSL |
| | | | | KSRVTMSVDTSKNQFSLKLNSVTAADTAVYYC |
| | | | | AREGQWRVPAQYYYFGMDVWGQGTTVTVSS |
| 949. | MSLN_13319- | artificial | Aa | EIVMTQSPGTLSLSPGERATLSCRASQSVSSS |
| | B8_CC - VL | | | YLAWYQQKPGQAPRLLIYGASSRATGIPDRFS |
| | | | | GSGSGTDFTLTISRLEPEDFAVYYCQQYGSSI |
| | | | | TFGCGTRLEIK |
| 950. | MSLN_18025- | artificial | Aa | SSSYFWV |
| | E3_CC - HCDR1 | | | |
| 951. | MSLN_18025- | artificial | Aa | SIYYSGSTYYNPSLKS |
| | E3_CC - HCDR2 | | | |
| 952. | MSLN_18025- | artificial | Aa | LPRGDRMTFDI |
| | E3_CC - HCDR3 | | | |
| 953. | MSLN_18025- | artificial | Aa | RASQSVSSSYLA |
| | E3_CC - LCDR1 | | | |
| 954. | MSLN_18025- | artificial | Aa | GASSRAT |
| | E3_CC - LCDR2 | | | |
| 955. | MSLN_18025- | artificial | Aa | QQYGSSPFT |
| | E3_CC - LCDR3 | | | |
| 956. | MSLN_18025- | artificial | Aa | QVQLQESGPGLVKPSETLSLTCTVSGGSISSS |
| | E3_CC - VH | | | SYFWVWIRQPPGKCLEWIGSIYYSGSTYYNPS |
| | | | | LKSRVTISVDTSKNQFSLKLNSVTAADTAVYY |
| | | | | CARLPRGDRMTFDIWGQGTMVTVSS |
| 957. | MSLN_18025- | artificial | Aa | EIVLTQSPGTLSLSPGERATLSCRASQSVSSS |
| | E3_CC - VL | | | YLAWYQQKPGQAPRLLIYGASSRATGIPDRFS |
| | | | | GSGSGTDFTLTISRLEPEDFAVYYCQQYGSSP |
| | | | | FTFGCGTKLEIK |
| 958. | MSLN_18026- | artificial | Aa | SYGMH |
| | C1_CC - | | | |
| | HCDR1 | | | |
| 959. | MSLN_18026- | artificial | Aa | VIWNRYSNKYYADAVKG |
| | C1_CC - | | | |
| | HCDR2 | | | |
| 960. | MSLN_18026- | artificial | Aa | DVPYYYGMDV |
| | C1_CC - | | | |
| | HCDR3 | | | |
| 961. | MSLN_18026- | artificial | Aa | TRSSGSIGDNYVQ |
| | C1_CC - LCDR1 | | | |
| 962. | MSLN_18026- | artificial | Aa | ENNQRPS |
| | C1_CC - LCDR2 | | | |
| 963. | MSLN_18026- | artificial | Aa | QSYHGSNVV |
| | C1_CC - LCDR3 | | | |
| 964. | MSLN_18026- | artificial | Aa | QVQLVESGGGVVLPGRSLRLSCAASGFPFSS |
| | C1_CC - VH | | | YGMHWVRQAPGKCLEWVAVIWNRYSNKYYA |
| | | | | DAVKGRFTISRDNSKNTLYLQMNSLRAEDTAV |
| | | | | YYCARDVPYYYGMDVWGQGTTVTVSS |
| 965. | MSLN_18026- | artificial | Aa | NFMLTQPHSVSESPGKTVIISCTRSSGSIGDNY |
| | C1_CC - VL | | | VQWYQQRPGSSPTTVIYENNQRPSGVPDRFS |
| | | | | GSIDSSSNSASLTISGLKTEDEADYYCQSYHG |
| | | | | SNVVFGCGTKLTVL |
| 966. | MSLN_18035- | artificial | Aa | SYGMH |
| | B6_CC - HCDR1 | | | |
| 967. | MSLN_18035- | artificial | Aa | VIWNDASNKYYADAVKG |
| | B6_CC - HCDR2 | | | |
| 968. | MSLN_18035- | artificial | Aa | DVPYYYGMDV |
| | B6_CC - HCDR3 | | | |
| 969. | MSLN_18035- | artificial | Aa | TRSSGSIGDNYVQ |
| | B6_CC - LCDR1 | | | |
| 970. | MSLN_18035- | artificial | Aa | ENNQRPS |
| | B6_CC - LCDR2 | | | |
| 971. | MSLN_18035- | artificial | Aa | QSYQQSNVV |
| | B6_CC - LCDR3 | | | |
| 972. | MSLN_18035- | artificial | Aa | QVQLVESGGGVVLPGRSLRLSCAASGFPFSS |
| | B6_CC - VH | | | YGMHWVRQAPGKCLEWVAVIWNDASNKYYA |
| | | | | DAVKGRFTISRDNSKNTLYLQMNSLRAEDTAV |
| | | | | YYCARDVPYYYGMDVWGQGTTVTVSS |
| 973. | MSLN_18035- | artificial | Aa | NFMLTQPHSVSESPGKTVIISCTRSSGSIGDNY |
| | B6_CC - VL | | | VQWYQQRPGSSPTTVIYENNQRPSGVPDRFS |
| | | | | GSIDSSSNSASLTISGLKTEDEADYYCQSYQQ |
| | | | | SNVVFGCGTKLTVL |
| 974. | MSLN_18036- | artificial | Aa | SYAMS |
| | C10_CC - | | | |
| | HCDR1 | | | |
| 975. | MSLN_18036- | artificial | Aa | AISGSGEFSYYAAAVKG |
| | C10_CC - | | | |
| | HCDR2 | | | |
| 976. | MSLN_18036- | artificial | Aa | VRNYYGSGSLDY |
| | C10_CC - | | | |
| | HCDR3 | | | |
| 977. | MSLN_18036- | artificial | Aa | RASQSVSSTYLA |
| | C10_CC - | | | |
| | LCDR1 | | | |
| 978. | MSLN_18036- | artificial | Aa | GASIRAT |
| | C10_CC - | | | |
| | LCDR2 | | | |
| 979. | MSLN_18036- | artificial | Aa | QQYGSSLT |
| | C10_CC - | | | |
| | LCDR3 | | | |
| 980. | MSLN_18036- | artificial | Aa | EVQLLESGGGVVQPGRSLRLSCAASGFTFSS |
| | C10_CC - VH | | | YAMSWVRQAPGKCLEWVSAISGSGEFSYYAA |
| | | | | AVKGRFTISRDNSKNTLYLQMNSLRAEDTAVY |
| | | | | YCAKVRNYYGSGSLDYWGQGTLVTVSS |
| 981. | MSLN_18036- | artificial | Aa | EIVLTQSPGTLSLSPGERATLSCRASQSVSST |
| | C10_CC - VL | | | YLAWYQQKPGQAPRLLIYGASIRATGIPDRFS |
| | | | | GSGSGTDFTLTISRLEPEDFAVYYCQQYGSSL |
| | | | | TFGCGTKVEIK |
| 982. | MSLN_18036- | artificial | Aa | SYAMS |
| | C5_CC - | | | |
| | HCDR1 | | | |
| 983. | MSLN_18036- | artificial | Aa | AISGSGEQWYYAPSVKG |
| | C5_CC - | | | |
| | HCDR2 | | | |
| 984. | MSLN_18036- | artificial | Aa | VRNYYGSGSLDY |
| | C5_CC - | | | |
| | HCDR3 | | | |
| 985. | MSLN_18036- | artificial | Aa | RASQSFSSAYLA |
| | C5_CC - LCDR1 | | | |
| 986. | MSLN_18036- | artificial | Aa | GASIRAT |
| | C5_CC - LCDR2 | | | |
| 987. | MSLN_18036- | artificial | Aa | QQYGSSLT |
| | C5_CC - LCDR3 | | | |
| 988. | MSLN_18036- | artificial | Aa | EVQLLESGGGVVQPGRSLRLSCAASGFTFSS |
| | C5_CC - VH | | | YAMSWVRQAPGKCLEWVSAISGSGEQWYYA |
| | | | | PSVKGRFTISRDNSKNTLYLQMNSLRAEDTAV |
| | | | | YYCAKVRNYYGSGSLDYWGQGTLVTVSS |
| 989. | MSLN_18036- | artificial | Aa | EIVLTQSPGTLSLSPGERATLSCRASQSFSSA |
| | C5_CC - VL | | | YLAWYQQKPGQAPRLLIYGASIRATGIPDRFS |
| | | | | GSGSGTDFTLTISRLEPEDFAVYYCQQYGSSL |
| | | | | TFGCGTKVEIK |
| 990. | MSLN_18037- | artificial | Aa | SYAMS |
| | B3_CC - HCDR1 | | | |
| 991. | MSLN_18037- | artificial | Aa | AISGSGGSTYYAPSVKG |
| | B3_CC - HCDR2 | | | |
| 992. | MSLN_18037- | artificial | Aa | EGYYPVSGYPLYYYFGMDV |
| | B3_CC - HCDR3 | | | |
| 993. | MSLN_18037- | artificial | Aa | RASQSVSSSYLA |
| | B3_CC - LCDR1 | | | |
| 994. | MSLN_18037- | artificial | Aa | GASSRAT |
| | B3_CC - LCDR2 | | | |
| 995. | MSLN_18037- | artificial | Aa | QQYGSSPIFT |
| | B3_CC - LCDR3 | | | |
| 996. | MSLN_18037- | artificial | Aa | EVQLLESGGGLVQPGGSLRLSCAASGFTFSS |
| | B3_CC - VH | | | YAMSWVRQAPGKCLEWVSAISGSGGSTYYA |
| | | | | PSVKGRFTISRDNSKNTLYLQMNSLRAEDTAV |
| | | | | YYCAKEGYYPVSGYPLYYYFGMDVWGQGTT |
| | | | | VTVSS |
| 997. | MSLN_18037- | artificial | Aa | EIVLTQSPGTLSLSPGERATLSCRASQSVSSS |
| | B3_CC - VL | | | YLAWYQQKPGQAPRLLIYGASSRATGIPDRFS |
| | | | | GSGSGTDFTLTISRLEPEDFAVYYCQQYGSSP |
| | | | | IFTFGCGTKVEIK |
| 998. | MSLN_18037- | artificial | Aa | SYAMS |
| | G4_CC - | | | |
| | HCDR1 | | | |
| 999. | MSLN_18037- | artificial | Aa | AISGSGGSTYYAGNVKG |
| | G4_CC - | | | |
| | HCDR2 | | | |
| 1000. | MSLN_18037- | artificial | Aa | EGYYPTSGYPLYYYFGMDV |
| | G4_CC - | | | |
| | HCDR3 | | | |
| 1001. | MSLN_18037- | artificial | Aa | RASQSVSSSYLA |
| | G4_CC - LCDR1 | | | |
| 1002. | MSLN_18037- | artificial | Aa | GASSRAT |
| | G4_CC - LCDR2 | | | |
| 1003. | MSLN_18037- | artificial | Aa | QQYGSSPIFT |
| | G4_CC - LCDR3 | | | |
| 1004. | MSLN_18037- | artificial | Aa | EVQLLESGGGLVQPGGSLRLSCAASGFTFSS |
| | G4_CC - VH | | | YAMSWVRQAPGKCLEWVSAISGSGGSTYYA |
| | | | | GNVKGRFTISRDNSKNTLYLQMNSLRAEDTAV |
| | | | | YYCAKEGYYPTSGYPLYYYFGMDVWGQGTT |
| | | | | VTVSS |
| 1005. | MSLN_18037- | artificial | Aa | EIVLTQSPGTLSLSPGERATLSCRASQSVSSS |
| | G4_CC - VL | | | YLAWYQQKPGQAPRLLIYGASSRATGIPDRFS |
| | | | | GSGSGTDFTLTISRLEPEDFAVYYCQQYGSSP |
| | | | | IFTFGCGTKVEIK |
| 1006. | MSLN_18126- | artificial | Aa | SYGMH |
| | H2_CC - | | | |
| | HCDR1 | | | |
| 1007. | MSLN_18126- | artificial | Aa | VIGSRESNKNYAESVKG |
| | H2_CC - | | | |
| | HCDR2 | | | |
| 1008. | MSLN_18126- | artificial | Aa | ALRIAVAASYYYYGLDV |
| | H2_CC - | | | |
| | HCDR3 | | | |
| 1009. | MSLN_18126- | artificial | Aa | RASQSVRSFLN |
| | H2_CC - LCDR1 | | | |
| 1010. | MSLN_18126- | artificial | Aa | TASSLQS |
| | H2_CC - LCDR2 | | | |
| 1011. | MSLN_18126- | artificial | Aa | QQSYEMPIT |
| | H2_CC - LCDR3 | | | |
| 1012. | MSLN_18126- | artificial | Aa | QVQLVESGGGVVQPGRSLRLSCAASGFTFSS |
| | H2_CC - VH | | | YGMHWVRQAPGKCLEWVAVIGSRESNKNYA |
| | | | | ESVKGRFTISRDNSKNTLYLQMNSLRAEDTAV |
| | | | | YYCASALRIAVAASYYYYGLDVWGQGTTVTV |
| | | | | SS |
| 1013. | MSLN_18126- | artificial | Aa | EIQMTQSPSSLSASVGDRVTITCRASQSVRSF |
| | H2_CC - VL | | | LNWYQQKPGKAPKLLIFTASSLQSGVPSRFSG |
| | | | | SGSGTDFTLTISSLQPEDFATYYCQQSYEMPI |
| | | | | TFGCGTRLEIK |
| 1014. | MSLN_18183- | artificial | Aa | SYGMG |
| | C2_CC - | | | |
| | HCDR1 | | | |
| 1015. | MSLN_18183- | artificial | Aa | VISYEASNKYYAEAVKG |
| | C2_CC - | | | |
| | HCDR2 | | | |
| 1016. | MSLN_18183- | artificial | Aa | EGAHFGSGSYYPLYYYYAMDV |
| | C2_CC - | | | |
| | HCDR3 | | | |
| 1017. | MSLN_18183- | artificial | Aa | RASQSVSSSYLA |
| | C2_CC - LCDR1 | | | |
| 1018. | MSLN_18183- | artificial | Aa | GASIRAT |
| | C2_CC - LCDR2 | | | |
| 1019. | MSLN_18183- | artificial | Aa | QQYGSSPIFT |
| | C2_CC - LCDR3 | | | |
| 1020. | MSLN_18183- | artificial | Aa | QVQLVESGGGVVQPGRSLRLSCAASGFTFSS |
| | C2_CC - VH | | | YGMGWVRQAPGKCLEWVAVISYEASNKYYA |
| | | | | EAVKGRFTISRDNSKNTLYLQMNSLRAEDTAV |
| | | | | YYCAREGAHFGSGSYYPLYYYYAMDVWGQG |
| | | | | TTVTVSS |
| 1021. | MSLN_18183- | artificial | Aa | EIVLTQSPGTLSLSPGERATLSCRASQSVSSS |
| | C2_CC - VL | | | YLAWYQQKPGQAPRLLIYGASIRATGIPDRFS |
| | | | | GSGSGTDFTLTISRLEPEDFAVYYCQQYGSSP |
| | | | | IFTFGCGTKVEIK |
| 1022. | MSLN_18201- | artificial | Aa | DYYMT |
| | G11_CC - | | | |
| | HCDR1 | | | |
| 1023. | MSLN_18201- | artificial | Aa | YISSSGSTIYYAEAVKG |
| | G11_CC - | | | |
| | HCDR2 | | | |
| 1024. | MSLN_18201- | artificial | Aa | DRNSHFDY |
| | G11_CC - | | | |
| | HCDR3 | | | |
| 1025. | MSLN_18201- | artificial | Aa | RASQGIRTWLA |
| | G11_CC - | | | |
| | LCDR1 | | | |
| 1026. | MSLN_18201- | artificial | Aa | GASGLQS |
| | G11_CC - | | | |
| | LCDR2 | | | |
| 1027. | MSLN_18201- | artificial | Aa | QQAESFPRT |
| | G11_CC - | | | |
| | LCDR3 | | | |
| 1028. | MSLN_18201- | artificial | Aa | QVQLVESGGGLVKPGGSLRLSCAASGFTFSD |
| | G11_CC - VH | | | YYMTWIRQAPGKCLEWLSYISSSGSTIYYAEA |
| | | | | VKGRFTISRDNAKNSLFLQMNSLRAEDTAVYY |
| | | | | CARDRNSHFDYWGQGTLVTVSS |
| 1029. | MSLN_18201- | artificial | Aa | EIQMTQSPSSVSASVGDRVTITCRASQGIRTW |
| | G11_CC - VL | | | LAWYQQKPGKAPKLLIYGASGLQSGVPSRFS |
| | | | | GSGSGTDFTLTISSLQPEDFATYYCQQAESFP |
| | | | | RTFGCGTKVEIK |
| 1030. | MSLN_MS_R4L | artificial | Aa | GYYIH |
| | _CC - HCDR1 | | | |
| 1031. | MSLN_MS_R4L | artificial | Aa | WINPNSGGTNYAQKFQG |
| | _CC - HCDR2 | | | |
| 1032. | MSLN_MS_R4L | artificial | Aa | VEAVAGREYYYFSGMDV |
| | _CC - HCDR3 | | | |
| 1033. | MSLN_MS_R4L | artificial | Aa | SGEKLGDKYVY |
| | _CC - LCDR1 | | | |
| 1034. | MSLN_MS_R4L | artificial | Aa | QSTKRPS |
| | _CC - LCDR2 | | | |
| 1035. | MSLN_MS_R4L | artificial | Aa | QAYHASTAV |
| | _CC - LCDR3 | | | |
| 1036. | MSLN_MS_R4L | artificial | Aa | QVQLVQSGAEVKKPGASVKVSCKASGYTFTG |
| | _CC - VH | | | YYIHWVRQAPGQCLEWMGWINPNSGGTNYA |
| | | | | QKFQGRVTMTRDTSISTAYMELSRLRSDDTAV |
| | | | | YYCARVEAVAGREYYYFSGMDVWGQGTTVT |
| | | | | VSS |
| 1037. | MSLN_MS_R4L | artificial | Aa | SYELTQPPSVSVSPGQTASITCSGEKLGDKYV |
| | _CC - VL | | | YWYQQKPGQSPVLVIYQSTKRPSGVPERFSG |
| | | | | SNSGNTATLTISGTQAMDEADYYCQAYHAST |
| | | | | AVFGCGTKLTVL |
| 1038. | MSLN_R195L_C | artificial | Aa | SYAMS |
| | C - HCDR1 | | | |
| 1039. | MSLN_R195L_C | artificial | Aa | AISGSGEFSYYAAAVKG |
| | C - HCDR2 | | | |
| 1040. | MSLN_R195L_C | artificial | Aa | VRNYYGSGSLDY |
| | C - HCDR3 | | | |
| 1041. | MSLN_R195L_C | artificial | Aa | RASQSVSSTYLA |
| | C - LCDR1 | | | |
| 1042. | MSLN_R195L_C | artificial | Aa | GASIRAT |
| | C - LCDR2 | | | |
| 1043. | MSLN_R195L_C | artificial | Aa | QQYQSSLT |
| | C - LCDR3 | | | |
| 1044. | MSLN_R195L_C | artificial | Aa | EVQLLESGGGVVQPGRSLRLSCAASGFTFSS |
| | C - VH | | | YAMSWVRQAPGKCLEWVSAISGSGEFSYYAA |
| | | | | AVKGRFTISRDNSKNTLYLQMNSLRAEDTAVY |
| | | | | YCAKVRNYYGSGSLDYWGQGTLVTVSS |
| 1045. | MSLN_R195L_C | artificial | Aa | EIVLTQSPGTLSLSPGERATLSCRASQSVSST |
| | C - VL | | | YLAWYQQKPGQAPRLLIYGASIRATGIPDRFS |
| | | | | GSGSGTDFTLTISRLEPEDFAVYYCQQYQSSL |
| | | | | TFGCGTKVEIK |
| 1046. | scFv_anti_CDH1 | artificial | Aa | SYGMH |
| | 9_2G6.007 CC - | | | |
| | HCDR1 | | | |
| 1047. | scFv_anti_CDH1 | artificial | Aa | FIWYEGSNKYYAESVKD |
| | 9_2G6.007 CC- | | | |
| | HCDR2 | | | |
| 1048. | scFv_anti_CDH1 | artificial | Aa | RAGIIGTIGYYYGMDV |
| | 9_2G6.007 CC- | | | |
| | HCDR3 | | | |
| 1049. | scFv_anti_CDH1 | artificial | Aa | SGDRLGEKYTS |
| | 9_2G6.007 CC- | | | |
| | LCDR1 | | | |
| 1050. | scFv_anti_CDH1 | artificial | Aa | QDTKRPS |
| | 9_2G6.007 CC- | | | |
| | LCDR2 | | | |
| 1051. | scFv_anti_CDH1 | artificial | Aa | QAWESSTVV |
| | 9_2G6.007 CC- | | | |
| | LCDR3 | | | |
| 1052. | CDH19_2G6.00 | artificial | Aa | QVQLVESGGGVVQPGGSLRLSCAASGFTFSS |
| | 7 CC- VH | | | YGMHWVRQAPGKCLEWVAFIWYEGSNKYYA |
| | | | | ESVKDRFTISRDNSKNTLYLQMNSLRAEDTAV |
| | | | | YYCARRAGIIGTIGYYYGMDVWGQGTTVTVSS |
| 1053. | CDH19_2G6.00 | artificial | Aa | SYELTQPPSVSVSPGQTASITCSGDRLGEKYT |
| | 7 CC- VL | | | SWYQQRPGQSPLLVIYQDTKRPSGIPERFSG |
| | | | | SNSGNTATLTISGTQAMDEADYYCQAWESST |
| | | | | VVFGCGTKLTVLS |
| 1054. | scFv_anti_FOLR | artificial | Aa | SNSVIWN |
| | 1_C145209_(2A | | | |
| | 3) - HCDR1 | | | |
| 1055. | scFv_anti_FOLR | artificial | Aa | RTYYRSKWYNDYAVSVKS |
| | 1_C145209_(2A | | | |
| | 3) - HCDR2 | | | |
| 1056. | scFv_anti_FOLR | artificial | Aa | TVYYYGMDV |
| | 1_C145209_(2A | | | |
| | 3) - HCDR3 | | | |
| 1057. | scFv_anti_FOLR | artificial | Aa | SGDKLGNNYAA |
| | 1_C145209_(2A | | | |
| | 3) - LCDR1 | | | |
| 1058. | scFv_anti_FOLR | artificial | Aa | QDSKRPS |
| | 1_C145209_(2A | | | |
| | 3) - LCDR2 | | | |
| 1059. | scFv_anti_FOLR | artificial | Aa | QSWDSSTVV |
| | 1_C145209_(2A | | | |
| | 3) - LCDR3 | | | |
| 1060. | FOLR1_C14520 | artificial | Aa | QVQLQQSGPGLVKPSQTLSLTCAISGDSVSS |
| | 9_(2A3) - VH | | | NSVIWNWIRQSPSRGLEWLGRTYYRSKWYN |
| | | | | DYAVSVKSRITINPDTSKNQFSLQLNSVTPEDT |
| | | | | AVYYCAGTVYYYGMDVWGQGATVTVSS |
| 1061. | FOLR1_C14520 | artificial | Aa | SYELTQPPSVSVSPGQTGSITCSGDKLGNNY |
| | 9_(2A3) - VL | | | AAWYQQKPGQSPVLVIYQDSKRPSGIPERFS |
| | | | | GSNSGNTATLTISGTQAVDEADYYCQSWDSS |
| | | | | TVVFGGGTKLTVLGS |
| 1062. | scFv_anti_MSL | artificial | Aa | SGANYWT |
| | N_C147862_(6F | | | |
| | 12) - HCDR1 | | | |
| 1063. | scFv_anti_MSL | artificial | Aa | YIYYSGSTYLNPSLRG |
| | N_C147862_(6F | | | |
| | 12) - HCDR2 | | | |
| 1064. | scFv_anti_MSL | artificial | Aa | ESGSSYGFDY |
| | N_C147862_(6F | | | |
| | 12) - HCDR3 | | | |
| 1065. | scFv_anti_MSL | artificial | Aa | RTSQSITSYLN |
| | N_C147862_(6F | | | |
| | 12) - LCDR1 | | | |
| 1066. | scFv_anti_MSL | artificial | Aa | ASSSLQS |
| | N_C147862_(6F | | | |
| | 12) - LCDR2 | | | |
| 1067. | scFv_anti_MSL | artificial | Aa | QQSYSGPFT |
| | N_C147862_(6F | | | |
| | 12) - LCDR3 | | | |
| 1068. | MSLN_C147862 | artificial | Aa | QVQLQESGPGLVKPSQTLSLTCTVSGGSISSG |
| | _(6F12) - VH | | | ANYWTWIRQHPGKGLEWIGYIYYSGSTYLNPS |
| | | | | LRGRVTMSVDTSKNQFSLKLSSVTAADTAVYY |
| | | | | CARESGSSYGFDYWGQGTLVTVSS |
| 1069. | MSLN_C147862 | artificial | Aa | DIQMTQSPSSLSASVGDRVTITCRTSQSITSYL |
| | _(6F12) - VL | | | NWYQQKPGQAPKLLIYASSSLQSGVPSRFSG |
| | | | | SGSGTDFTLTISSLQPEDFATYYCQQSYSGPF |
| | | | | TFGPGTKVDIKRS |
| 1070. | CH3 15-E11 CC | artificial | na | CAGGTTCAGTTGGTTCAGTCTGGCGCCGAA |
| | x I2Lopt x G4 x | | | GTGAAGAAACCAGGCGCTTCTGTGAAGGTG |
| | scFc SEFL2 | | | TCCTGCAAGGCCTCTGGCTACACCTTTACCA |
| | clipopt x G4 x | | | ACTACTGGATGAACTGGGTCCGACAGGCTC |
| | MS 15-B12 CC x | | | CTGGCCAGTGTCTGGAATGGATGGGCAATA |
| | I2L_GQ - | | | TCGCTTACGGCGTGAAGGGCACCAACTACA |
| | Nucleotide | | | ACCAGAAATTCCAGGGCAGAGTGACCATGA |
| | Sequence | | | CCGTGGACACCTCTTCCTCCACCGCCTACA |
| | | | | TGGAACTGTCCCGGCTGAGATCTGACGACA |
| | | | | CCGCCGTGTACTACTGCGCCACCAGATACT |
| | | | | TCTACGTGATGGACTATTGGGGCCAGGGCA |
| | | | | CCCTGGTTACAGTTTCTTCTGGCGGCGGAG |
| | | | | GACAAGGCGGTGGTGGTCAAGGCGGAGGC |
| | | | | GGACAGGATATCCAGATGACCCAGTCTCCT |
| | | | | TCCAGCCTGTCTGCCTCTGTGGGCGACAGA |
| | | | | GTGACAATCACCTGTCGGGCCTCTCAGGAC |
| | | | | ATCTCCAACTACCTGAACTGGTATCAGCAGA |
| | | | | AACCCGGCAAGGTGCCCAAGCTGCTGATCT |
| | | | | ACTACACCTCCAGACTGCACTCCGGCGTGC |
| | | | | CCTCTAGATTTTCTGGCTCTGGATCTGGCAC |
| | | | | CGACTTCACCCTGACCATCAGTTCTCTGCAG |
| | | | | CCTGAGGACGTGGCCACCTACTACTGTGTG |
| | | | | CAGTACGCCCAGTTTCCTCTGACCTTCGGC |
| | | | | TGTGGCACCAAGGTGGAAATCAAAAGCGGT |
| | | | | GGCGGAGGCCAAGAGGTGCAGCTTGTTGAA |
| | | | | TCTGGCGGAGGATTGGTGCAGCCTGGCGG |
| | | | | ATCTCTGAAGCTGTCTTGTGCCGCCTCCGG |
| | | | | CTTCACCTTCAACAAATACGCCATGAATTGG |
| | | | | GTTCGACAAGCCCCAGGCAAAGGCATGGAA |
| | | | | TGGGTCGCCCGGATCAGATCCAAGTACAAC |
| | | | | AACTACGCTACCTACTACGCCGACGCCGTG |
| | | | | AAGGACAGATTCACCATCTCTCGGGACGAC |
| | | | | TCCAAGAACACCCTGTACCTGCAGATGAAC |
| | | | | AACCTGAAAACCGAGGATACCGCCGTCTAT |
| | | | | TACTGTGTCAGAGCCGGCAACTTCGGCTCC |
| | | | | TCCTACATCTCCTACTTTGCCTACTGGGGAC |
| | | | | AGGGAACCCTCGTGACTGTTTCTAGCGGTG |
| | | | | GTGGCGGACAAGGTGGCGGTGGACAAGGC |
| | | | | GGCGGAGGCCAACAAACAGTGGTCACACAA |
| | | | | GAGCCCAGCCTGACAGTGTCTCCTGGCGGA |
| | | | | ACAGTGACCATCACATGTGGATCTTCTACCG |
| | | | | GCGCTGTGACCTCCGGCAACTACCCCAATT |
| | | | | GGATCCAGAAGAAGCCCGGCCAGGCTCCTA |
| | | | | GAGGACTGATCGGCGGAACAAAGTTTCTGG |
| | | | | CCCCTGGCACACCAGCCAGATTCTCAGGAT |
| | | | | CTCTGGAAGGCGGCAAGGCCGCTCTGACAT |
| | | | | TGTCTGGCGTTCAGCCAGAGGATGAGGCCG |
| | | | | AGTACTATTGCGTGCTGTACTACTCCAACAG |
| | | | | ATGGGTGTTCGGCTCCGGCACAAAGCTGAC |
| | | | | AGTTCTCGGAGGTGGCGGATGCCCTCCTTG |
| | | | | TCCTGCTCCTGAATTGCTCGGCGGACCCTC |
| | | | | CGTGTTCCTGTTTCCTCCAAAGCCTAAGGAC |
| | | | | ACCCTGATGATCTCTCGGACCCCTGAAGTG |
| | | | | ACCTGCGTGGTGGTGGATGTGTCCCACGAG |
| | | | | GAACCAGAAGTGAAGTTCAATTGGTACGTG |
| | | | | GACGGCGTGGAAGTGCACAACGCTAAGACC |
| | | | | AAGCCTTGCGAGGAACAGTACGGCAGCACC |
| | | | | TACAGATGTGTGTCCGTGCTGACCGTGCTG |
| | | | | CACCAGGACTGGCTGAATGGCAAAGAGTAC |
| | | | | AAGTGCAAGGTGTCCAACAAGGCACTGCCC |
| | | | | GCTCCTATCGAAAAGACCATCTCCAAGGCTA |
| | | | | AGGGCCAGCCTCGGGAACCTCAGGTTTACA |
| | | | | CCCTGCCTCCATCTCGGGAAGAGATGACCA |
| | | | | AGAACCAGGTGTCCCTGACCTGCCTGGTCA |
| | | | | AGGGCTTCTACCCTTCCGATATCGCCGTGG |
| | | | | AATGGGAGTCCAATGGCCAGCCTGAGAACA |
| | | | | ACTACAAGACCACACCTCCTGTGCTGGACT |
| | | | | CCGACGGCTCATTCTTCCTGTACTCCAAGCT |
| | | | | GACTGTGGACAAGTCTCGGTGGCAGCAGG |
| | | | | GCAACGTGTTCTCCTGTTCTGTGATGCACGA |
| | | | | GGCCCTGCACAACCACTACACCCAGAAGTC |
| | | | | CCTGTCTCTGAGCCCTGGCAAAGGTGGTGG |
| | | | | CGGTCAAGGCGGTGGTGGCCAAGGCGGCG |
| | | | | GAGGACAAGGTGGCGGAGGCCAAGGTGGT |
| | | | | GGCGGACAAGGCGGAGGTGGTCAATGTCC |
| | | | | TCCTTGTCCAGCACCAGAACTCCTCGGAGG |
| | | | | CCCTTCTGTGTTTCTGTTCCCACCTAAGCCA |
| | | | | AAGGATACACTCATGATCAGCAGGACTCCC |
| | | | | GAAGTGACATGTGTCGTCGTGGACGTTTCC |
| | | | | CATGAAGAACCCGAAGTCAAGTTTAATTGGT |
| | | | | ATGTCGATGGCGTCGAGGTCCACAATGCCA |
| | | | | AGACAAAGCCCTGTGAAGAACAATACGGGT |
| | | | | CCACCTATAGATGCGTCAGCGTCCTGACAG |
| | | | | TCCTGCATCAGGATTGGCTCAACGGGAAAG |
| | | | | AATACAAATGTAAAGTCTCTAACAAGGCTCT |
| | | | | CCCAGCACCAATCGAGAAAACCATTAGCAA |
| | | | | GGCCAAAGGACAGCCCCGCGAGCCACAAG |
| | | | | TGTATACCCTGCCACCTAGCCGCGAGGAAA |
| | | | | TGACAAAGAATCAAGTCTCTCTGACCTGTCT |
| | | | | CGTGAAGGGGTTTTACCCCAGCGACATTGC |
| | | | | CGTCGAGTGGGAGTCTAACGGACAACCCGA |
| | | | | AAACAATTATAAGACAACCCCACCTGTCCTG |
| | | | | GACAGCGACGGCTCATTTTTTCTCTACTCTA |
| | | | | AACTCACCGTGGATAAGTCCAGATGGCAAC |
| | | | | AGGGAAATGTGTTCAGCTGCAGCGTGATGC |
| | | | | ATGAAGCTCTCCACAATCATTATACCCAGAA |
| | | | | AAGCCTGAGCTTGTCTCCCGGCAAAGGTGG |
| | | | | CGGAGGACAGGTTCAGTTGCAAGAGTCTGG |
| | | | | ACCTGGCCTCGTGAAGCCTTCTGAGACACT |
| | | | | GAGCCTGACCTGTACCGTGTCTGGCGGCTC |
| | | | | CATCTCCTCCAGCTCTTACTTCTGGGGCTG |
| | | | | GATCAGACAGCCTCCAGGCAAGTGCCTCGA |
| | | | | GTGGATCGGCAACATCTACTACTCCGGCTC |
| | | | | CAGCAACTACAATCCTAGCCTGAAGTCCCG |
| | | | | CGTGACAATCTCTGTGGATACCTCTAAGAAC |
| | | | | CAGTTTAGCCTCAAGCTGTCCAGCGTGACC |
| | | | | GCCGCTGATACCGCTGTGTATTATTGCGCTA |
| | | | | GACTGCCCAGAGGCGACCGGGATGCTTTCG |
| | | | | ATATTTGGGGACAAGGCACAATGGTCACCG |
| | | | | TTTCTAGCGGAGGCGGTGGCCAAGGTGGC |
| | | | | GGAGGCCAAGGCGGCGGTGGTCAAGATATT |
| | | | | GTGATGACACAGAGCCCCTCTAGCCTGAGC |
| | | | | GCTTCCGTGGGAGATCGCGTGACCATTACC |
| | | | | TGTAGAGCCAGCCAGGGCATCAGCAATTAC |
| | | | | CTGGCCTGGTATCAACAAAAGCCTGGGAAA |
| | | | | GTCCCTAAGCTCCTCATCTACGCCGCTTCCA |
| | | | | CACTGCAGAGCGGCGTGCCAAGCAGATTCA |
| | | | | GTGGATCCGGCAGCGGAACCGACTTTACTC |
| | | | | TGACTATCTCCAGCCTGCAGCCAGAAGATTT |
| | | | | CGCTACCTATTACTGCCAGCAGTCCTACAG |
| | | | | CACCCCTTTCACCTTTGGCTGCGGAACTAA |
| | | | | GGTCGAGATCAAGAGCGGAGGTGGTGGAC |
| | | | | AAGAGGTCCAGTTGGTCGAGTCAGGTGGCG |
| | | | | GCTTGGTCCAACCAGGTGGAAGCCTGAAAC |
| | | | | TGAGCTGCGCCGCTTCTGGGTTTACTTTTAA |
| | | | | CAAATATGCTATGAACTGGGTTCGCCAGGC |
| | | | | ACCTGGAAAAGGCATGGAATGGGTTGCCAG |
| | | | | AATCCGCAGCAAGTATAACAATTATGCCACC |
| | | | | TATTATGCCGATGCTGTCAAGGATCGGTTCA |
| | | | | CCATCAGCAGGGACGATAGCAAGAATACCC |
| | | | | TCTATCTCCAAATGAACAATCTCAAGACAGA |
| | | | | GGACACAGCAGTGTACTATTGTGTTCGCGC |
| | | | | TGGCAACTTTGGCAGCAGCTACATCAGCTA |
| | | | | CTTCGCTTACTGGGGCCAAGGGACACTTGT |
| | | | | GACCGTTAGCAGCGGAGGCGGAGGACAAG |
| | | | | GTGGCGGAGGACAAGGCGGAGGTGGACAG |
| | | | | CAGACAGTTGTGACCCAAGAGCCTTCTCTG |
| | | | | ACTGTGTCACCAGGCGGCACCGTGACAATT |
| | | | | ACATGCGGAAGTTCCACAGGCGCCGTGACC |
| | | | | AGCGGCAATTATCCTAACTGGATTCAGAAAA |
| | | | | AACCTGGACAGGCCCCAAGAGGCCTGATTG |
| | | | | GAGGCACCAAATTTCTCGCTCCCGGCACTC |
| | | | | CTGCTCGGTTCTCTGGTAGTCTTGAAGGCG |
| | | | | GAAAAGCTGCCCTGACTCTCTCTGGCGTGC |
| | | | | AACCCGAAGATGAAGCTGAATATTACTGCGT |
| | | | | CCTCTACTATAGCAATCGCTGGGTTTTCGGA |
| | | | | AGCGGCACCAAGCTCACTGTCCTCTGA |
| 1071. | CH3 15- | artificial | na | CAGGTGCAGCTGGTTCAGTCTGGCGCCGAA |
| | E11_1_VAG_CC | | | GTGAAGAAACCTGGCGCCTCTGTGAAGGTG |
| | x I2L x G4 x | | | TCCTGCAAGGCTTCTGGCTACACCTTTACCA |
| | scFc x G4 x MS | | | ACTACTGGATGAACTGGGTCCGACAGGCCC |
| | 15-B12 CC x I2L | | | CTGGCCAGTGTTTGGAGTGGATGGGCAATA |
| | clipopt_DI, | | | TCGCTTACGGCGTGGCCGGCACCAACTACA |
| | AMG305 - | | | ACCAGAAATTCCAGGGCAGAGTGACAATGA |
| | Nucleotide | | | CCGTGGACACCTCCTCCTCCACCGCCTACA |
| | Sequence | | | TGGAACTGTCCCGGCTGAGATCTGACGACA |
| | | | | CCGCCGTGTACTACTGCGCCACCAGATACT |
| | | | | TCTACGTGATGGACTACTGGGGCCAGGGCA |
| | | | | CCCTGGTTACAGTTTCTTCTGGCGGCGGAG |
| | | | | GACAAGGCGGAGGTGGTCAAGGTGGTGGC |
| | | | | GGACAGGATATCCAGATGACCCAGTCTCCT |
| | | | | TCCAGCCTGTCTGCCTCTGTGGGCGACAGA |
| | | | | GTGACCATCACCTGTAGAGCCAGCCAGGAC |
| | | | | ATCTCCAACTACCTGAACTGGTATCAGCAGA |
| | | | | AACCCGGCAAGGTGCCCAAGCTGCTGATCT |
| | | | | ACTACACCTCTCGGCTGCACTCTGGCGTGC |
| | | | | CCTCTAGATTTTCTGGCTCCGGCTCTGGCA |
| | | | | CCGACTTTACCCTGACAATCTCCAGCCTGCA |
| | | | | GCCTGAGGATGTGGCCACCTACTACTGTGT |
| | | | | GCAGTACGCCCAGTTTCCTCTGACCTTCGG |
| | | | | CTGTGGCACCAAGGTGGAAATCAAGTCTGG |
| | | | | AGGCGGAGGCCAAGAGGTGCAGCTGGTGG |
| | | | | AGTCCGGCGGCGGCCTGGTGCAGCCCGGC |
| | | | | GGCTCCCTGAAGCTGTCCTGCGCCGCCTCC |
| | | | | GGCTTCACCTTCAACAAGTACGCCATGAACT |
| | | | | GGGTGAGGCAGGCCCCCGGCAAGGGCATG |
| | | | | GAGTGGGTGGCCAGGATCAGGTCCAAGTAC |
| | | | | AACAACTACGCCACCTACTACGCCGACGCC |
| | | | | GTGAAGGACAGGTTCACCATCTCCAGGGAC |
| | | | | GACTCCAAGAACACCCTGTACCTGCAGATG |
| | | | | AACAACCTGAAGACCGAGGACACCGCCGTG |
| | | | | TACTACTGCGTGAGGGCCGGCAACTTCGGC |
| | | | | TCCTCCTACATCTCCTACTTCGCCTACTGGG |
| | | | | GCCAGGGCACCCTGGTGACCGTGTCCTCC |
| | | | | GGCGGCGGCGGCCAAGGCGGCGGCGGCC |
| | | | | AAGGCGGCGGCGGCCAACAGACCGTGGTG |
| | | | | ACCCAGGAGCCCTCCCTGACCGTGTCCCCC |
| | | | | GGCGGCACCGTGACCATCACCTGCGGCTC |
| | | | | CTCCACCGGCGCCGTGACCTCCGGCAACTA |
| | | | | CCCCAACTGGATCCAGAAGAAGCCCGGCCA |
| | | | | GGCCCCCAGGGGCCTGATCGGCGGCACCA |
| | | | | AGTTCCTGGCCCCCGGCACCCCCGCCAGG |
| | | | | TTCTCCGGCTCCCTGGAGGGCGGCAAGGC |
| | | | | CGCCCTGACCCTGTCCGGCGTGCAGCCCG |
| | | | | AGGACGAGGCCGAGTACTACTGCGTGCTGT |
| | | | | ACTACTCCAACAGGTGGGTGTTCGGCTCCG |
| | | | | GCACCAAGCTGACCGTCCTAGGCGGAGGC |
| | | | | GGCTGCCCTCCTTGTCCTGCTCCTGAATTG |
| | | | | CTCGGCGGACCCTCCGTGTTCCTGTTTCCT |
| | | | | CCAAAGCCTAAGGACACCCTGATGATCTCT |
| | | | | CGTACGCCTGAAGTGACCTGCGTGGTGGTG |
| | | | | GATGTGTCCCACGAGGAACCCGAAGTGAAG |
| | | | | TTCAATTGGTACGTGGACGGCGTGGAAGTG |
| | | | | CACAACGCCAAGACAAAGCCCTGCGAGGAA |
| | | | | CAGTACGGCTCCACCTACAGATGCGTGTCC |
| | | | | GTGCTGACAGTGCTGCACCAGGATTGGCTG |
| | | | | AACGGCAAAGAGTACAAGTGCAAGGTGTCC |
| | | | | AACAAGGCCCTGCCTGCTCCTATCGAAAAG |
| | | | | ACCATCTCCAAGGCCAAGGGCCAGCCTAGA |
| | | | | GAGCCCCAGGTTTACACCCTGCCTCCAAGC |
| | | | | AGAGAAGAGATGACCAAGAACCAGGTGTCC |
| | | | | CTGACCTGCCTGGTCAAGGGCTTCTACCCT |
| | | | | TCCGATATCGCCGTGGAATGGGAGAGCAAT |
| | | | | GGACAGCCCGAGAACAACTACAAGACCACA |
| | | | | CCTCCTGTGCTGGACTCCGACGGCTCATTC |
| | | | | TTCCTGTACTCCAAGCTGACCGTGGACAAG |
| | | | | TCCAGATGGCAGCAGGGCAACGTGTTCTCC |
| | | | | TGCTCCGTGATGCACGAGGCCCTGCACAAT |
| | | | | CACTACACCCAGAAGTCCCTGTCTCTGTCC |
| | | | | CCTGGAAAAGGAGGCGGAGGACAAGGCGG |
| | | | | AGGTGGTCAAGGTGGTGGTGGCCAAGGCG |
| | | | | GAGGCGGACAAGGCGGCGGAGGACAAGGT |
| | | | | GGCGGTGGACAGTGTCCTCCATGTCCAGCA |
| | | | | CCTGAGCTTCTCGGAGGCCCTTCTGTGTTT |
| | | | | CTGTTCCCACCTAAGCCAAAGGATACACTCA |
| | | | | TGATCAGCCGCACACCTGAAGTCACATGTG |
| | | | | TCGTCGTGGATGTCTCTCATGAAGAACCAG |
| | | | | AAGTCAAGTTTAATTGGTATGTCGATGGCGT |
| | | | | CGAGGTCCACAATGCTAAGACCAAGCCTTG |
| | | | | TGAAGAACAATATGGCAGCACCTATCGCTGT |
| | | | | GTGTCTGTCCTGACCGTCCTGCATCAAGAC |
| | | | | TGGCTCAATGGGAAAGAATACAAATGCAAA |
| | | | | GTCTCTAACAAAGCTCTGCCCGCACCAATC |
| | | | | GAGAAAACCATCAGCAAGGCTAAAGGACAG |
| | | | | CCTCGCGAGCCTCAAGTGTATACCCTGCCA |
| | | | | CCTTCTCGCGAGGAAATGACAAAAAATCAAG |
| | | | | TCTCCCTCACCTGTCTCGTGAAGGGATTCTA |
| | | | | TCCCAGCGACATTGCCGTCGAGTGGGAGTC |
| | | | | TAATGGCCAGCCTGAAAACAATTATAAGACA |
| | | | | ACCCCACCTGTCCTGGACAGCGACGGCTCA |
| | | | | TTTTTTCTCTACTCTAAACTCACCGTGGATAA |
| | | | | GAGCCGGTGGCAACAGGGAAATGTGTTCAG |
| | | | | CTGTAGCGTGATGCATGAAGCTCTCCACAA |
| | | | | CCATTATACACAGAAGAGTCTGAGCCTGTCT |
| | | | | CCTGGCAAAGGCGGCGGAGGACAGGTGCA |
| | | | | ACTCCAGGAATCCGGGCCAGGGTTGGTGAA |
| | | | | ACCCAGCGAGACACTGTCTCTGACTTGCAC |
| | | | | TGTTTCTGGTGGCTCCATTTCCTCTAGCTCT |
| | | | | TACTTCTGGGGTTGGATACGGCAACCACCT |
| | | | | GGGAAGTGTCTCGAATGGATTGGTAACATC |
| | | | | TACTATAGTGGATCCTCCAACTACAATCCCA |
| | | | | GCCTGAAGAGTCGTGTGACTATCAGCGTTG |
| | | | | ACACCTCAAAGAATCAGTTCTCCCTTAAGCT |
| | | | | GAGTTCCGTGACAGCAGCAGATACAGCCGT |
| | | | | CTACTACTGTGCTCGACTTCCTAGGGGAGA |
| | | | | TCGGGATGCCTTCGACATTTGGGGTCAGGG |
| | | | | TACGATGGTAACAGTGTCTAGTGGAGGCGG |
| | | | | AGGTCAAGGCGGCGGAGGCCAAGGAGGAG |
| | | | | GCGGACAAGATATCGTGATGACCCAGAGCC |
| | | | | CATCAAGCCTGAGTGCTAGCGTTGGGGACA |
| | | | | GGGTCACTATCACTTGCAGAGCCTCACAGG |
| | | | | GGATTTCCAACTATCTGGCCTGGTATCAGCA |
| | | | | GAAACCTGGCAAGGTCCCCAAACTCCTGAT |
| | | | | ATATGCTGCAAGCACGCTGCAAAGCGGGGT |
| | | | | ACCCTCTCGCTTTTCTGGGTCTGGCTCTGG |
| | | | | CACAGACTTTACCCTGACCATCTCCAGTTTG |
| | | | | CAGCCTGAGGACTTTGCCACCTACTATTGC |
| | | | | CAGCAGTCCTACTCAACACCCTTCACCTTTG |
| | | | | GCTGTGGCACCAAGGTGGAGATCAAATCCG |
| | | | | GAGGCGGAGGACAAGAAGTCCAGCTGGTT |
| | | | | GAAAGTGGTGGCGGATTGGTTCAGCCAGGC |
| | | | | GGCTCTCTGAAGCTGTCTTGTGCTGCCTCC |
| | | | | GGCTTCACCTTCAACAAATACGCCATGAATT |
| | | | | GGGTTCGACAAGCCCCAGGCAAAGGCATG |
| | | | | GAATGGGTCGCCCGGATCAGATCCAAGTAC |
| | | | | AACAACTACGCTACCTACTACGCCGACGCC |
| | | | | GTGAAGGACCGGTTCACCATCTCCAGAGAT |
| | | | | GACTCCAAGAACACCCTGTACCTGCAGATG |
| | | | | AACAACCTCAAGACCGAGGATACCGCCGTC |
| | | | | TATTACTGTGTCAGAGCCGGCAACTTCGGC |
| | | | | TCCTCCTACATCTCCTACTTCGCCTACTGGG |
| | | | | GCCAGGGAACCCTTGTGACAGTCTCTAGTG |
| | | | | GCGGTGGTGGTCAAGGTGGTGGCGGCCAA |
| | | | | GGCGGTGGCGGACAACAAACAGTGGTCAC |
| | | | | CCAAGAGCCTAGCCTGACCGTTTCTCCTGG |
| | | | | CGGCACCGTGACCATCACATGCGGATCTTC |
| | | | | TACCGGCGCTGTGACCTCCGGCAACTACCC |
| | | | | CAATTGGATCCAGAAGAAGCCAGGCCAGGC |
| | | | | TCCTAGAGGACTGATCGGCGGCACAAAGTT |
| | | | | TCTGGCTCCCGGCACTCCCGCCAGATTTTC |
| | | | | TGGATCTCTGGAAGGCGGCAAGGCTGCTCT |
| | | | | GACATTGTCTGGCGTCCAGCCAGAGGATGA |
| | | | | GGCCGAGTACTATTGCGTGCTGTACTACTC |
| | | | | CAACAGATGGGTGTTCGGCTCCGGCACCAA |
| | | | | GCTGACAGTCCTATGA |
| 1072. | MS 83-C2 CC x | artificial | na | CAGGTGCAGCTGGTGGAATCTGGTGGCGG |
| | I2L x scFc x EP | | | AGTTGTGCAGCCTGGCAGATCCCTGAGACT |
| | 71-A5 CC x I2L | | | GTCTTGTGCCGCCTCCGGCTTCACCTTCTC |
| | (G4S)3 - | | | CTCTTATGGAATGGGCTGGGTCCGACAGGC |
| | COMBI#11 | | | CCCTGGCAAATGTTTGGAATGGGTCGCCGT |
| | (A8P) - | | | GATCTCCTACGAGGCCTCCAACAAGTACTA |
| | Nucleotide | | | CGCCGAGGCCGTGAAGGGCAGATTCACCAT |
| | Sequence | | | CTCCAGAGACAACTCCAAGAACACCCTGTA |
| | | | | CCTGCAGATGAACTCCCTGAGAGCCGAGGA |
| | | | | CACCGCCGTGTACTACTGTGCTAGAGAGGG |
| | | | | CGCCCATTTCGGCTCCGGCTCTTACTACCC |
| | | | | TCTGTACTACTACTACGCTATGGACGTGTGG |
| | | | | GGCCAGGGCACCACAGTGACAGTTTCTAGC |
| | | | | GGAGGCGGAGGAAGTGGGGGGGGAGGATC |
| | | | | TGGCGGTGGTGGTTCTGAAATCGTGCTGAC |
| | | | | CCAGTCTCCTGGCACACTGTCTTTGAGCCC |
| | | | | TGGCGAGAGAGCTACCCTGAGCTGTAGAGC |
| | | | | CTCTCAGTCCGTGTCCTCCTCTTACCTGGCC |
| | | | | TGGTATCAGCAGAAGCCCGGCCAGGCTCCT |
| | | | | AGACTGTTGATCTACGGCGCCTCCATCAGA |
| | | | | GCCACAGGCATCCCTGATAGATTCTCCGGC |
| | | | | AGCGGCTCTGGCACCGACTTCACCCTGACA |
| | | | | ATCTCTCGGCTGGAACCCGAGGACTTTGCT |
| | | | | GTGTACTATTGCCAGCAGTACGGCAGCTCC |
| | | | | CCTATCTTCACCTTTGGCTGCGGCACCAAG |
| | | | | GTGGAAATCAAGTCCGGGGGGGGAGGCTC |
| | | | | CGAGGTGCAGCTGGTGGAGTCCGGCGGCG |
| | | | | GCCTGGTGCAGCCCGGCGGCTCCCTGAAG |
| | | | | CTGTCCTGCGCCGCCTCCGGCTTCACCTTC |
| | | | | AACAAGTACGCCATGAACTGGGTGAGGCAG |
| | | | | GCCCCCGGCAAGGGCATGGAGTGGGTGGC |
| | | | | CAGGATCAGGTCCAAGTACAACAACTACGC |
| | | | | CACCTACTACGCCGACGCCGTGAAGGACAG |
| | | | | GTTCACCATCTCCAGGGACGACTCCAAGAA |
| | | | | CACCCTGTACCTGCAGATGAACAACCTGAA |
| | | | | GACCGAGGACACCGCCGTGTACTACTGCGT |
| | | | | GAGGGCCGGCAACTTCGGCTCCTCCTACAT |
| | | | | CTCCTACTTCGCCTACTGGGGCCAGGGCAC |
| | | | | CCTGGTGACCGTGTCCTCCGGCGGCGGCG |
| | | | | GCTCCGGCGGCGGCGGCTCCGGCGGCGG |
| | | | | CGGCTCCCAGACCGTGGTGACCCAGGAGC |
| | | | | CCTCCCTGACCGTGTCCCCCGGCGGCACC |
| | | | | GTGACCATCACCTGCGGCTCCTCCACCGGC |
| | | | | GCCGTGACCTCCGGCAACTACCCCAACTGG |
| | | | | ATCCAGAAGAAGCCCGGCCAGGCCCCCAG |
| | | | | GGGCCTGATCGGCGGCACCAAGTTCCTGG |
| | | | | CCCCCGGCACCCCCGCCAGGTTCTCCGGC |
| | | | | TCCCTGGAGGGCGGCAAGGCCGCCCTGAC |
| | | | | CCTGTCCGGCGTGCAGCCCGAGGACGAGG |
| | | | | CCGAGTACTACTGCGTGCTGTACTACTCCAA |
| | | | | CAGGTGGGTGTTCGGCTCCGGCACCAAGCT |
| | | | | GACCGTGCTAGGCGGCGGAGGATCTGGCG |
| | | | | GAGGTGGAAGCGGAGGCGGTGGATCTGAC |
| | | | | AAGACCCACACATGTCCTCCATGTCCCGCC |
| | | | | CCTGAACTGCTAGGCGGACCTAGCGTGTTC |
| | | | | CTGTTCCCCCCAAAGCCCAAGGACACCCTG |
| | | | | ATGATCAGCCGTACGCCCGAAGTGACCTGC |
| | | | | GTGGTGGTGGATGTGTCCCACGAGGACCCT |
| | | | | GAAGTGAAGTTCAATTGGTACGTGGACGGC |
| | | | | GTGGAAGTGCACAACGCCAAGACCAAGCCC |
| | | | | TGCGAGGAACAGTACGGCAGCACCTACAGA |
| | | | | TGCGTGTCCGTGCTGACCGTGCTGCATCAG |
| | | | | GACTGGCTGAACGGCAAAGAGTACAAGTGC |
| | | | | AAGGTGTCCAACAAGGCCCTGCCTGCCCCC |
| | | | | ATCGAGAAAACCATCAGCAAGGCCAAGGGC |
| | | | | CAGCCCCGCGAGCCTCAAGTGTATACCCTG |
| | | | | CCCCCTAGCCGGGAAGAGATGACCAAGAAC |
| | | | | CAGGTGTCCCTGACCTGTCTCGTGAAGGGC |
| | | | | TTCTACCCCTCCGATATCGCCGTGGAATGG |
| | | | | GAGAGCAACGGCCAGCCCGAGAACAACTAC |
| | | | | AAGACCACCCCCCCTGTGCTGGACAGCGAC |
| | | | | GGCTCATTCTTCCTGTACTCCAAACTGACCG |
| | | | | TGGACAAGAGCCGGTGGCAGCAGGGCAAC |
| | | | | GTGTTCAGCTGCAGCGTGATGCACGAGGCC |
| | | | | CTGCACAACCACTACACCCAGAAGTCCCTG |
| | | | | TCCCTGTCTCCCGGGAAAGGGGGGGGAGG |
| | | | | ATCTGGCGGAGGCGGATCTGGGGGGGGAG |
| | | | | GAAGTGGGGGAGGGGGAAGCGGAGGGGG |
| | | | | AGGCTCAGGGGGGGGAGGATCCGATAAGA |
| | | | | CCCACACCTGTCCCCCTTGCCCTGCCCCTG |
| | | | | AACTGCTGGGAGGCCCTAGCGTGTTCCTGT |
| | | | | TCCCCCCAAAGCCCAAGGACACCCTGATGA |
| | | | | TCAGCCGGACCCCCGAAGTGACCTGCGTG |
| | | | | GTGGTGGATGTGTCCCACGAGGACCCTGAA |
| | | | | GTGAAGTTCAATTGGTACGTGGACGGCGTG |
| | | | | GAAGTGCACAACGCCAAGACCAAGCCCTGC |
| | | | | GAGGAACAGTACGGCAGCACCTACAGATGC |
| | | | | GTGTCCGTGCTGACCGTGCTGCACCAGGAC |
| | | | | TGGCTGAACGGCAAAGAGTACAAGTGCAAG |
| | | | | GTGTCCAACAAGGCCCTGCCTGCCCCCATC |
| | | | | GAGAAAACCATCAGCAAGGCCAAGGGCCAG |
| | | | | CCCCGCGAGCCTCAAGTGTATACCCTGCCC |
| | | | | CCCAGCCGGGAAGAGATGACCAAGAACCAG |
| | | | | GTGTCCCTGACCTGTCTCGTGAAGGGCTTC |
| | | | | TACCCCTCCGATATCGCCGTGGAATGGGAG |
| | | | | AGCAACGGCCAGCCCGAGAACAACTACAAG |
| | | | | ACCACCCCCCCTGTGCTGGACAGCGACGG |
| | | | | CTCATTCTTCCTGTACTCCAAGCTGACAGTG |
| | | | | GACAAGTCTAGATGGCAGCAGGGCAACGTG |
| | | | | TTCAGCTGCAGCGTGATGCACGAGGCCCTG |
| | | | | CACAACCACTACACCCAGAAGTCCCTGTCC |
| | | | | CTGAGCCCCGGCAAAGGTGGAGGCGGATC |
| | | | | TGGCGGTGGCGGGAGTGGAGGAGGAGGCA |
| | | | | GCCAGGTGCAGCTGATGGAATCTGGTGGCG |
| | | | | GAGTTGTGCAGCCTGGCAGATCCCTGAGAC |
| | | | | TGTCTTGTGCCGCCTCCGGCTTCACCTTCA |
| | | | | GCCGGTACTATATGCACTGGGTCCGACAGG |
| | | | | CCCCTGGCAAGTGTCCTGAATGGGTTGCCG |
| | | | | TGATCTGGCACGACGGCTCCAACAAGTACT |
| | | | | ACGCCGACTCCGTGAAGGGCAGATTCACCA |
| | | | | TCTCTCGGGACAACTCCAAGAACACCCTGT |
| | | | | ACCTGCAGATGAACTCCCTGAGAGCCGAGG |
| | | | | ACACCGCCGTGTACTACTGTGCTAGAGAGG |
| | | | | CCCCTTCTCTGGCCTATTGGGGACAGGGAA |
| | | | | CACTGGTCACAGTGTCCTCTGGCGGCGGAG |
| | | | | GATCTGGCGGAGGTGGTAGCGGAGGCGGT |
| | | | | GGATCTGAGATCGTGATGACCCAGTCTCCT |
| | | | | GGCACACTGTCTCTGAGCCCTGGCGAGAGA |
| | | | | GCTACCCTGTCTTGTAGAGCCTCTCAGTCC |
| | | | | GTGTCCTCCTCCTACCTGGCTTGGTATCAG |
| | | | | CAGAAGCCAGGCCAGGCTCCTCGGCTGTTG |
| | | | | ATCTACGGCGCTTCCTCTAGAGCCACAGGC |
| | | | | ATCCCTGACAGATTCTCCGGCTCTGGCTCT |
| | | | | GGCACCGACTTCACCCTGACCATCTCCAGA |
| | | | | CTGGAACCCGAGGACTTTGCTGTGTACTATT |
| | | | | GCCAGCAGTACGGCTCCTCCATCACCTTCG |
| | | | | GCTGTGGCACCAGGCTGGAAATCAAGTCTG |
| | | | | GAGGCGGAGGATCTGAAGTCCAGCTGGTTG |
| | | | | AAAGTGGTGGCGGATTGGTTCAGCCAGGCG |
| | | | | GCTCTCTGAAGCTGTCTTGTGCTGCCTCCG |
| | | | | GCTTCACCTTCAACAAATACGCCATGAATTG |
| | | | | GGTTCGACAAGCCCCAGGCAAAGGCATGGA |
| | | | | ATGGGTCGCCCGGATCAGATCCAAGTACAA |
| | | | | CAACTACGCTACCTACTACGCCGACGCCGT |
| | | | | GAAGGACCGGTTCACCATCTCCAGAGATGA |
| | | | | CTCCAAGAACACCCTGTACCTGCAGATGAA |
| | | | | CAACCTCAAGACCGAGGATACCGCCGTCTA |
| | | | | TTACTGTGTCAGAGCCGGCAACTTCGGCTC |
| | | | | CTCCTACATCTCCTACTTCGCCTACTGGGGC |
| | | | | CAGGGAACCCTTGTGACAGTCTCTAGTGGC |
| | | | | GGTGGTGGTAGTGGTGGTGGCGGCTCAGG |
| | | | | CGGTGGCGGATCTCAAACAGTGGTCACCCA |
| | | | | AGAGCCTAGCCTGACCGTTTCTCCTGGCGG |
| | | | | CACCGTGACCATCACATGCGGATCTTCTAC |
| | | | | CGGCGCTGTGACCTCCGGCAACTACCCCAA |
| | | | | TTGGATCCAGAAGAAGCCAGGCCAGGCTCC |
| | | | | TAGAGGACTGATCGGCGGCACAAAGTTTCT |
| | | | | GGCTCCCGGCACTCCCGCCAGATTTTCTGG |
| | | | | ATCTCTGGAAGGCGGCAAGGCTGCTCTGAC |
| | | | | ATTGTCTGGCGTCCAGCCAGAGGATGAGGC |
| | | | | CGAGTACTATTGCGTGCTGTACTACTCCAAC |
| | | | | AGATGGGTGTTCGGCTCCGGCACCAAGCTG |
| | | | | ACAGTCCTATGA |
| 1073. | MS 83-C2 CC x | artificial | Aa | QVQLVESGGGVVQPGRSLRLSCAASGFTFSS |
| | I2L x scFc x EP | | | YGMGWVRQAPGKCLEWVAVISYEASNKYYA |
| | 71-A5 CC x I2L | | | EAVKGRFTISRDNSKNTLYLQMNSLRAEDTAV |
| | (G4S)3 - | | | YYCAREGAHFGSGSYYPLYYYYAMDVWGQG |
| | COMBI#11 | | | TTVTVSSGGGGSGGGGSGGGGSEIVLTQSP |
| | (A8P) | | | GTLSLSPGERATLSCRASQSVSSSYLAWYQQ |
| | | | | KPGQAPRLLIYGASIRATGIPDRFSGSGSGTDF |
| | | | | TLTISRLEPEDFAVYYCQQYGSSPIFTFGCGTK |
| | | | | VEIKSGGGGSEVQLVESGGGLVQPGGSLKLS |
| | | | | CAASGFTFNKYAMNWVRQAPGKGMEWVARI |
| | | | | RSKYNNYATYYADAVKDRFTISRDDSKNTLYL |
| | | | | QMNNLKTEDTAVYYCVRAGNFGSSYISYFAY |
| | | | | WGQGTLVTVSSGGGGSGGGGSGGGGSQTV |
| | | | | VTQEPSLTVSPGGTVTITCGSSTGAVTSGNYP |
| | | | | NWIQKKPGQAPRGLIGGTKFLAPGTPARFSGS |
| | | | | LEGGKAALTLSGVQPEDEAEYYCVLYYSNRW |
| | | | | VFGSGTKLTVLGGGGSGGGGSGGGGSDKTH |
| | | | | TCPPCPAPELLGGPSVFLFPPKPKDTLMISRT |
| | | | | PEVTCVVVDVSHEDPEVKFNWYVDGVEVHNA |
| | | | | KTKPCEEQYGSTYRCVSVLTVLHQDWLNGKE |
| | | | | YKCKVSNKALPAPIEKTISKAKGQPREPQVYTL |
| | | | | PPSREEMTKNQVSLTCLVKGFYPSDIAVEWES |
| | | | | NGQPENNYKTTPPVLDSDGSFFLYSKLTVDKS |
| | | | | RWQQGNVFSCSVMHEALHNHYTQKSLSLSP |
| | | | | GKGGGGSGGGGSGGGGSGGGGSGGGGSG |
| | | | | GGGSDKTHTCPPCPAPELLGGPSVFLFPPKP |
| | | | | KDTLMISRTPEVTCVVVDVSHEDPEVKFNWYV |
| | | | | DGVEVHNAKTKPCEEQYGSTYRCVSVLTVLH |
| | | | | QDWLNGKEYKCKVSNKALPAPIEKTISKAKGQ |
| | | | | PREPQVYTLPPSREEMTKNQVSLTCLVKGFY |
| | | | | PSDIAVEWESNGQPENNYKTTPPVLDSDGSF |
| | | | | FLYSKLTVDKSRWQQGNVFSCSVMHEALHNH |
| | | | | YTQKSLSLSPGKGGGGSGGGGSGGGGSQV |
| | | | | QLMESGGGVVQPGRSLRLSCAASGFTFSRYY |
| | | | | MHWVRQAPGKCPEWVAVIWHDGSNKYYADS |
| | | | | VKGRFTISRDNSKNTLYLQMNSLRAEDTAVYY |
| | | | | CAREAPSLAYWGQGTLVTVSSGGGGSGGGG |
| | | | | SGGGGSEIVMTQSPGTLSLSPGERATLSCRA |
| | | | | SQSVSSSYLAWYQQKPGQAPRLLIYGASSRA |
| | | | | TGIPDRFSGSGSGTDFTLTISRLEPEDFAVYYC |
| | | | | QQYGSSITFGCGTRLEIKSGGGGSEVQLVES |
| | | | | GGGLVQPGGSLKLSCAASGFTFNKYAMNWV |
| | | | | RQAPGKGMEWVARIRSKYNNYATYYADAVKD |
| | | | | RFTISRDDSKNTLYLQMNNLKTEDTAVYYCVR |
| | | | | AGNFGSSYISYFAYWGQGTLVTVSSGGGGSG |
| | | | | GGGSGGGGSQTVVTQEPSLTVSPGGTVTITC |
| | | | | GSSTGAVTSGNYPNWIQKKPGQAPRGLIGGT |
| | | | | KFLAPGTPARFSGSLEGGKAALTLSGVQPED |
| | | | | EAEYYCVLYYSNRWVFGSGTKLTVL |
| 1074. | CL1 9-G4 CC | artificial | na | CAGGTGCAGCTGGTTCAGTCTGGCGCCGAA |
| | x6H10.09 x scFc | | | GTGAAGAAACCTGGCGCCTCTGTGAAGGTG |
| | xFL 4-E9 CC | | | TCCTGCAAGGCTTCTGGCTACACCTTTACCG |
| | x6H10.09 - | | | ACTACTACATGCACTGGGTCCGACAGGCCC |
| | Nucleotide | | | CTGGCCAGTGTTTGGAATGGATGGGCTGGA |
| | Sequence | | | TCAACCCCAACTCTGGCGGCCCTAATTACG |
| | | | | CCCAGAAATTCCAGGGCAGAGTGACCATGA |
| | | | | CCAGAGACACCTCCATCTCCACCGCTCACA |
| | | | | TGGAACTGTCCCGGCTGAGATCTGACGACA |
| | | | | CCGCCGTGTACTACTGCGCCAGAGAAAAGC |
| | | | | ACGCTGTGGCCGGCATCGGCTTCGATTATT |
| | | | | GGGGACAGGGCACCCTGGTCACCGTTTCTA |
| | | | | GCGGAGGCGGAGGATCTGGTGGTGGTGGA |
| | | | | TCTGGCGGCGGAGGCTCTGATATCCAGATG |
| | | | | ACCCAGTCTCCTTCCTCCGTGTCTGCCTCTG |
| | | | | TGGGCGACAGAGTGACAATCACCTGTCAGG |
| | | | | CCAGCCAGGACATCTCCAACTACCTGAACT |
| | | | | GGTATCAGCAGAAGCCCGGCAAGGCCCCTA |
| | | | | AGCTGCTGATCTACGCTGCCTCCTCTCTGG |
| | | | | AATCTGGCGTGCCCTCCAGATTCTCCGGCT |
| | | | | CTGGCTCTGGCACAGACTTTACCCTGACAAT |
| | | | | CTCCAGCCTGCAGCCTGAGGACTTCGCCAC |
| | | | | CTACTACTGTCAGCAGGCCAACAGCTTCCC |
| | | | | TCTGACCTTTGGCTGTGGCACCAAGGTGGA |
| | | | | CATCAAGTCTGGTGGCGGCGGTTCCGAAGT |
| | | | | CCAGCTGGTTGAAAGTGGTGGCGGATTGGT |
| | | | | TCAGCCAGGCGGCTCTCTGAAGCTGTCTTG |
| | | | | TGCTGCCTCCGGCTTCACCTTCAACAAATAC |
| | | | | GCCATGAATTGGGTTCGACAAGCCCCAGGC |
| | | | | AAAGGCATGGAATGGGTCGCCCGGATCAGA |
| | | | | TCCAAGTACAACAACTACGCTACCTACTACG |
| | | | | CCGACGCCGTGAAGGACCGGTTCACCATCT |
| | | | | CCAGAGATGACTCCAAGAACACCCTGTACC |
| | | | | TGCAGATGAACAACCTCAAGACCGAGGATA |
| | | | | CCGCCGTCTATTACTGTGTCAGAGCCGGCA |
| | | | | ACTTCGGCTCCTCCTACATCTCCTACTTCGC |
| | | | | CTACTGGGGCCAGGGAACCCTTGTGACAGT |
| | | | | CTCTAGTGGCGGTGGTGGTAGTGGTGGTGG |
| | | | | CGGCTCAGGCGGTGGCGGATCTCAAACAGT |
| | | | | GGTCACCCAAGAGCCTAGCCTGACCGTTTC |
| | | | | TCCTGGCGGCACCGTGACCATCACATGCGG |
| | | | | ATCTTCTACCGGCGCTGTGACCTCCGGCAA |
| | | | | CTACCCCAATTGGATCCAGAAGAAGCCAGG |
| | | | | CCAGGCTCCTAGAGGACTGATCGGCGGCAC |
| | | | | AAAGTTTCTGGCTCCCGGCACTCCCGCCAG |
| | | | | ATTTTCTGGATCTCTGGAAGGCGGCAAGGC |
| | | | | TGCTCTGACATTGTCTGGCGTCCAGCCAGA |
| | | | | GGATGAGGCCGAGTACTATTGCGTGCTGTA |
| | | | | CTACTCCAACAGATGGGTGTTCGGCTCCGG |
| | | | | CACCAAGCTGACAGTCCTAGGCGGCGGAG |
| | | | | GATCTGGCGGAGGTGGAAGCGGAGGCGGT |
| | | | | GGATCTGACAAGACCCACACATGTCCTCCA |
| | | | | TGTCCCGCCCCTGAACTGCTAGGCGGACCT |
| | | | | AGCGTGTTCCTGTTCCCCCCAAAGCCCAAG |
| | | | | GACACCCTGATGATCAGCCGTACGCCCGAA |
| | | | | GTGACCTGCGTGGTGGTGGATGTGTCCCAC |
| | | | | GAGGACCCTGAAGTGAAGTTCAATTGGTAC |
| | | | | GTGGACGGCGTGGAAGTGCACAACGCCAA |
| | | | | GACCAAGCCCTGCGAGGAACAGTACGGCA |
| | | | | GCACCTACAGATGCGTGTCCGTGCTGACCG |
| | | | | TGCTGCATCAGGACTGGCTGAACGGCAAAG |
| | | | | AGTACAAGTGCAAGGTGTCCAACAAGGCCC |
| | | | | TGCCTGCCCCCATCGAGAAAACCATCAGCA |
| | | | | AGGCCAAGGGCCAGCCCCGCGAGCCTCAA |
| | | | | GTGTATACCCTGCCCCCTAGCCGGGAAGAG |
| | | | | ATGACCAAGAACCAGGTGTCCCTGACCTGT |
| | | | | CTCGTGAAGGGCTTCTACCCCTCCGATATC |
| | | | | GCCGTGGAATGGGAGAGCAACGGCCAGCC |
| | | | | CGAGAACAACTACAAGACCACCCCCCCTGT |
| | | | | GCTGGACAGCGACGGCTCATTCTTCCTGTA |
| | | | | CTCCAAACTGACCGTGGACAAGAGCCGGTG |
| | | | | GCAGCAGGGCAACGTGTTCAGCTGCAGCGT |
| | | | | GATGCACGAGGCCCTGCACAACCACTACAC |
| | | | | CCAGAAGTCCCTGTCCCTGTCTCCCGGGAA |
| | | | | AGGCGGCGGAGGATCTGGCGGAGGCGGAT |
| | | | | CTGGGGGGGGAGGAAGTGGGGGAGGGGG |
| | | | | AAGCGGAGGGGGAGGCTCAGGGGGGGGA |
| | | | | GGATCCGATAAGACCCACACCTGTCCCCCT |
| | | | | TGCCCTGCCCCTGAACTGCTGGGAGGCCCT |
| | | | | AGCGTGTTCCTGTTCCCCCCAAAGCCCAAG |
| | | | | GACACCCTGATGATCAGCCGGACCCCCGAA |
| | | | | GTGACCTGCGTGGTGGTGGATGTGTCCCAC |
| | | | | GAGGACCCTGAAGTGAAGTTCAATTGGTAC |
| | | | | GTGGACGGCGTGGAAGTGCACAACGCCAA |
| | | | | GACCAAGCCCTGCGAGGAACAGTACGGCA |
| | | | | GCACCTACAGATGCGTGTCCGTGCTGACCG |
| | | | | TGCTGCACCAGGACTGGCTGAACGGCAAAG |
| | | | | AGTACAAGTGCAAGGTGTCCAACAAGGCCC |
| | | | | TGCCTGCCCCCATCGAGAAAACCATCAGCA |
| | | | | AGGCCAAGGGCCAGCCCCGCGAGCCTCAA |
| | | | | GTGTATACCCTGCCCCCCAGCCGGGAAGAG |
| | | | | ATGACCAAGAACCAGGTGTCCCTGACCTGT |
| | | | | CTCGTGAAGGGCTTCTACCCCTCCGATATC |
| | | | | GCCGTGGAATGGGAGAGCAACGGCCAGCC |
| | | | | CGAGAACAACTACAAGACCACCCCCCCTGT |
| | | | | GCTGGACAGCGACGGCTCATTCTTCCTGTA |
| | | | | CTCCAAGCTGACAGTGGACAAGTCTAGATG |
| | | | | GCAGCAGGGCAACGTGTTCAGCTGCAGCGT |
| | | | | GATGCACGAGGCCCTGCACAACCACTACAC |
| | | | | CCAGAAGTCCCTGTCCCTGAGCCCCGGCAA |
| | | | | AGGTGGAGGCGGATCTGGCGGTGGCGGGA |
| | | | | GTGGAGGAGGAGGCAGCCAGGTGACTCTG |
| | | | | AAAGAATCCGGTCCCACTCTCGTCAAGCCT |
| | | | | ACCGAAACTCTGACCCTGACGTGTACTGTC |
| | | | | AGTGGGTTTTCCTTCAGGAATGCACGAATG |
| | | | | GGTGTAAGCTGGATACGCCAACCACCTGGC |
| | | | | AAATGCCTGGAATGGCTCGCTCACATCTTCA |
| | | | | GCAATGACGAGAAGTCCTATTCTACCTCCCT |
| | | | | GAAATCCCGGTTGACCATTTCCAAGGATAC |
| | | | | GAGCAAGTCTCAGGTTGTGCTGACCATGAC |
| | | | | CAACATGGATCCCGTGGATACAGCCACCTA |
| | | | | CTTCTGTGCTCGTGTTCCCGAGTATAGCTCT |
| | | | | GGCTGGTATCGGTTTGACTACTGGGGACAG |
| | | | | GGCACATTGGTGACAGTATCTTCAGGAGGC |
| | | | | GGCGGGTCAGGTGGCGGAGGATCAGGCGG |
| | | | | TGGTGGTTCTGACATTCAGATGACTCAGAG |
| | | | | CCCATCAAGTCTGAGTGCCAGTGTTGGAGA |
| | | | | TAGAGTGACCATCAGTTGCAGAGCCTCTCA |
| | | | | GTCTATCAGGAGCTACCTTAACTGGTATCAG |
| | | | | CAGAAACCCGGCAAAGCTCCTAAGCTGCTG |
| | | | | ATCTACGCAACTAGCAGCCTTCAAGGAGGG |
| | | | | GTGCCATCCCGCTTTAGTGGGTCAGGATCT |
| | | | | GGCACTGACTTTACCCTCACAATCAGCTCCT |
| | | | | TGCAACCTGAGGACTTTGCCACCTACTACTG |
| | | | | CCAGCAGTCCTATTCCACACCCTTCACATTC |
| | | | | GGGTGTGGGACAAAGGTCGAGATTAAGTCC |
| | | | | GGAGGCGGAGGATCTGAAGTGCAGCTGGTT |
| | | | | GAATCTGGCGGCGGATTGGTTCAGCCTGGC |
| | | | | GGATCTCTGAAGCTGTCTTGTGCCGCCTCT |
| | | | | GGCTTCACCTTCAACAAATACGCCATGAACT |
| | | | | GGGTCCGACAGGCCCCTGGCAAAGGCATG |
| | | | | GAATGGGTCGCCCGGATCAGATCCAAGTAC |
| | | | | AACAACTACGCTACCTACTACGCCGACGCC |
| | | | | GTGAAGGACCGGTTCACCATCTCCAGAGAT |
| | | | | GACTCCAAGAACACCCTGTACCTGCAGATG |
| | | | | AACAACCTCAAGACCGAGGACACCGCCGTG |
| | | | | TACTACTGTGTCAGAGCCGGCAACTTCGGC |
| | | | | TCCTCCTACATCTCCTACTTCGCCTATTGGG |
| | | | | GCCAGGGCACCCTGGTCACAGTTAGTTCAG |
| | | | | GTGGCGGTGGATCAGGCGGCGGAGGTTCT |
| | | | | GGTGGCGGAGGCTCTCAAACAGTGGTCACC |
| | | | | CAAGAGCCTAGCCTGACCGTTTCTCCTGGC |
| | | | | GGCACCGTGACCATCACCTGTGGATCTTCT |
| | | | | ACCGGCGCTGTGACCTCCGGCAACTACCCC |
| | | | | AATTGGATCCAGAAGAAGCCCGGCCAGGCT |
| | | | | CCTAGAGGACTGATCGGAGGCACCAAGTTT |
| | | | | CTGGCTCCCGGCACTCCTGCCAGATTCTCC |
| | | | | GGTTCTCTGGAAGGCGGAAAGGCCGCTCTG |
| | | | | ACATTGTCTGGCGTGCAGCCTGAGGATGAG |
| | | | | GCTGAGTACTACTGCGTGCTGTACTACTCCA |
| | | | | ACAGATGGGTGTTCGGCTCCGGCACCAAGC |
| | | | | TGACAGTGCTT |
| 1075. | MS 15-B12 CC | artificial | Aa | QVQLQESGPGLVKPSETLSLTCTVSGGSISSS |
| | x4F10.03 I2M | | | SYFWGWIRQPPGKCLEWIGNIYYSGSSNYNP |
| | xscFc xCH3 15- | | | SLKSRVTISVDTSKNQFSLKLSSVTAADTAVYY |
| | E11 CC | | | CARLPRGDRDAFDIWGQGTMVTVSSGGGGS |
| | x4F10.03 I2M - | | | GGGGSGGGGSDIVMTQSPSSLSASVGDRVTI |
| | Full Sequence | | | TCRASQGISNYLAWYQQKPGKVPKLLIYAAST |
| | | | | LQSGVPSRFSGSGSGTDFTLTISSLQPEDFAT |
| | | | | YYCQQSYSTPFTFGCGTKVEIKSGGGGSEVQ |
| | | | | LVESGGGLVQPGGSLKLSCAASGFTFNKYAM |
| | | | | NWVRQAPGKGMEWVARIRSKYNNYATYYAD |
| | | | | AVKDRFTISRDDSKNTLYLQMNNLKTEDTAVY |
| | | | | YCVRAGNFGTSYISYWAYWGQGTLVTVSSGG |
| | | | | GGSGGGGSGGGGSQTVVTQEPSLTVSPGGT |
| | | | | VTITCGSSTGAVTSGNYPNWVQKKPGQAPRG |
| | | | | LIGGTKFLAPGTPARFSGSLSGGKAALTLSGV |
| | | | | QPEDEAEYYCVLWYSNRWVFGSGTKLTVLG |
| | | | | GGGSGGGGSGGGGSDKTHTCPPCPAPELLG |
| | | | | GPSVFLFPPKPKDTLMISRTPEVTCVVVDVSH |
| | | | | EDPEVKFNWYVDGVEVHNAKTKPCEEQYGST |
| | | | | YRCVSVLTVLHQDWLNGKEYKCKVSNKALPA |
| | | | | PIEKTISKAKGQPREPQVYTLPPSREEMTKNQ |
| | | | | VSLTCLVKGFYPSDIAVEWESNGQPENNYKTT |
| | | | | PPVLDSDGSFFLYSKLTVDKSRWQQGNVFSC |
| | | | | SVMHEALHNHYTQKSLSLSPGKGGGGSGGG |
| | | | | GSGGGGSGGGGSGGGGSGGGGSDKTHTCP |
| | | | | PCPAPELLGGPSVFLFPPKPKDTLMISRTPEV |
| | | | | TCVVVDVSHEDPEVKFNWYVDGVEVHNAKTK |
| | | | | PCEEQYGSTYRCVSVLTVLHQDWLNGKEYKC |
| | | | | KVSNKALPAPIEKTISKAKGQPREPQVYTLPPS |
| | | | | REEMTKNQVSLTCLVKGFYPSDIAVEWESNG |
| | | | | QPENNYKTTPPVLDSDGSFFLYSKLTVDKSR |
| | | | | WQQGNVFSCSVMHEALHNHYTQKSLSLSPG |
| | | | | KGGGGSGGGGSGGGGSQVQLVQSGAEVKK |
| | | | | PGASVKVSCKASGYTFTNYWMNWVRQAPGQ |
| | | | | CLEWMGNIAYGVKGTNYNQKFQGRVTMTVD |
| | | | | TSSSTAYMELSRLRSDDTAVYYCATRYFYVM |
| | | | | DYWGQGTLVTVSSGGGGSGGGGSGGGGSDI |
| | | | | QMTQSPSSLSASVGDRVTITCRASQDISNYLN |
| | | | | WYQQKPGKVPKLLIYYTSRLHSGVPSRFSGS |
| | | | | GSGTDFTLTISSLQPEDVATYYCVQYAQFPLT |
| | | | | FGCGTKVEIKSGGGGSEVQLVESGGGLVQPG |
| | | | | GSLKLSCAASGFTFNKYAMNWVRQAPGKGM |
| | | | | EWVARIRSKYNNYATYYADAVKDRFTISRDDS |
| | | | | KNTLYLQMNNLKTEDTAVYYCVRAGNFGTSYI |
| | | | | SYWAYWGQGTLVTVSSGGGGSGGGGSGGG |
| | | | | GSQTVVTQEPSLTVSPGGTVTITCGSSTGAVT |
| | | | | SGNYPNWVQKKPGQAPRGLIGGTKFLAPGTP |
| | | | | ARFSGSLSGGKAALTLSGVQPEDEAEYYCVL |
| | | | | WYSNRWVFGSGTKLTVL |
| 1076. | MS 15-B12 CC x | artificial | Aa | QVQLQESGPGLVKPSETLSLTCTVSGGSISSS |
| | I2L x scFc xCH3 | | | SYFWGWIRQPPGKCLEWIGNIYYSGSSNYNP |
| | 15-E11 CC x | | | SLKSRVTISVDTSKNQFSLKLSSVTAADTAVYY |
| | I2M2 - Full | | | CARLPRGDRDAFDIWGQGTMVTVSSGGGGS |
| | Sequence | | | GGGGSGGGGSDIVMTQSPSSLSASVGDRVTI |
| | | | | TCRASQGISNYLAWYQQKPGKVPKLLIYAAST |
| | | | | LQSGVPSRFSGSGSGTDFTLTISSLQPEDFAT |
| | | | | YYCQQSYSTPFTFGCGTKVEIKSGGGGSEVQ |
| | | | | LVESGGGLVQPGGSLKLSCAASGFTFNKYAM |
| | | | | NWVRQAPGKGMEWVARIRSKYNNYATYYAD |
| | | | | AVKDRFTISRDDSKNTLYLQMNNLKTEDTAVY |
| | | | | YCVRAGNFGSSYISYFAYWGQGTLVTVSSGG |
| | | | | GGSGGGGSGGGGSQTVVTQEPSLTVSPGGT |
| | | | | VTITCGSSTGAVTSGNYPNWIQKKPGQAPRG |
| | | | | LIGGTKFLAPGTPARFSGSLEGGKAALTLSGV |
| | | | | QPEDEAEYYCVLYYSNRWVFGSGTKLTVLGG |
| | | | | GGSGGGGSGGGGSDKTHTCPPCPAPELLGG |
| | | | | PSVFLFPPKPKDTLMISRTPEVTCVVVDVSHE |
| | | | | DPEVKFNWYVDGVEVHNAKTKPCEEQYGSTY |
| | | | | RCVSVLTVLHQDWLNGKEYKCKVSNKALPAPI |
| | | | | EKTISKAKGQPREPQVYTLPPSREEMTKNQV |
| | | | | SLTCLVKGFYPSDIAVEWESNGQPENNYKTTP |
| | | | | PVLDSDGSFFLYSKLTVDKSRWQQGNVFSCS |
| | | | | VMHEALHNHYTQKSLSLSPGKGGGGSGGGG |
| | | | | SGGGGSGGGGSGGGGSGGGGSDKTHTCPP |
| | | | | CPAPELLGGPSVFLFPPKPKDTLMISRTPEVT |
| | | | | CVVVDVSHEDPEVKFNWYVDGVEVHNAKTKP |
| | | | | CEEQYGSTYRCVSVLTVLHQDWLNGKEYKCK |
| | | | | VSNKALPAPIEKTISKAKGQPREPQVYTLPPSR |
| | | | | EEMTKNQVSLTCLVKGFYPSDIAVEWESNGQ |
| | | | | PENNYKTTPPVLDSDGSFFLYSKLTVDKSRW |
| | | | | QQGNVFSCSVMHEALHNHYTQKSLSLSPGKG |
| | | | | GGGSGGGGSGGGGSQVQLVQSGAEVKKPG |
| | | | | ASVKVSCKASGYTFTNYWMNWVRQAPGQCL |
| | | | | EWMGNIAYGVKGTNYNQKFQGRVTMTVDTS |
| | | | | SSTAYMELSRLRSDDTAVYYCATRYFYVMDY |
| | | | | WGQGTLVTVSSGGGGSGGGGSGGGGSDIQ |
| | | | | MTQSPSSLSASVGDRVTITCRASQDISNYLNW |
| | | | | YQQKPGKVPKLLIYYTSRLHSGVPSRFSGSGS |
| | | | | GTDFTLTISSLQPEDVATYYCVQYAQFPLTFG |
| | | | | CGTKVEIKSGGGGSEVQLVESGGGLVQPGGS |
| | | | | LKLSCAASGFTFNKYAINWVREAPGKGLEWV |
| | | | | ARIRSKYNNYATYYADAVKDRFTISRDDSKNT |
| | | | | AYLQMNNLKTEDTAVYYCVRNANFGTSYISYF |
| | | | | AYWGQGTLVTVSSGGGGSGGGGSGGGGSQ |
| | | | | TVVTQEPSLTVSPGGTVTLTCGSSTGAVTSGN |
| | | | | YPNWVQKKPGQAPRGLIGGTKFLAPGTPARF |
| | | | | SGSLLGGKAALTLSGVQPEDEAEYYCVLWYS |
| | | | | NRWVFGSGTKLTVL |
| 1077. | MS 15-B12 CC x | artificial | Aa | QVQLQESGPGLVKPSETLSLTCTVSGGSISSSSY |
| | I2C x scFc xCH3 | | | FWGWIRQPPGKCLEWIGNIYYSGSSNYNPSLKS |
| | 15-E11 CCx I2C0 - | | | RVTISVDTSKNQFSLKLSSVTAADTAVYYCAR |
| | Full Sequence | | | LPRGDRDAFDIWGQGTMVTVSSGGGGSGGGG |
| | | | | SGGGGSDIVMTQSPSSLSASVGDRVTITCRASQ |
| | | | | GISNYLAWYQQKPGKVPKLLIYAASTLQSGVP |
| | | | | SRFSGSGSGTDFTLTISSLQPEDFATYYCQQSYS |
| | | | | TPFTFGCGTKVEIKSGGGGSEVQLVESGGGLV |
| | | | | QPGGSLKLSCAASGFTFNKYAMNWVRQAPGK |
| | | | | GLEWVARIRSKYNNYATYYADSVKDRFTISRD |
| | | | | DSKNTAYLQMNNLKTEDTAVYYCVRHGNFGN |
| | | | | SYISYWAYWGQGTLVTVSSGGGGSGGGGSGG |
| | | | | GGSQTVVTQEPSLTVSPGGTVTLTCGSSTGAVT |
| | | | | SGNYPNWVQQKPGQAPRGLIGGTKFLAPGTPA |
| | | | | RFSGSLLGGKAALTLSGVQPEDEAEYYCVLWY |
| | | | | SNRWVFGGGTKLTVLGGGGSGGGGSGGGGSD |
| | | | | KTHTCPPCPAPELLGGPSVFLFPPKPKDTLMISR |
| | | | | TPEVTCVVVDVSHEDPEVKFNWYVDGVEVHN |
| | | | | AKTKPCEEQYGSTYRCVSVLTVLHQDWLNGK |
| | | | | EYKCKVSNKALPAPIEKTISKAKGQPREPQVYT |
| | | | | LPPSREEMTKNQVSLTCLVKGFYPSDIAVEWES |
| | | | | NGQPENNYKTTPPVLDSDGSFFLYSKLTVDKS |
| | | | | RWQQGNVFSCSVMHEALHNHYTQKSLSLSPG |
| | | | | KGGGGSGGGGSGGGGSGGGGSGGGGSGGGGS |
| | | | | DKTHTCPPCPAPELLGGPSVFLFPPKPKDTLMIS |
| | | | | RTPEVTCVVVDVSHEDPEVKFNWYVDGVEVH |
| | | | | NAKTKPCEEQYGSTYRCVSVLTVLHQDWLNG |
| | | | | KEYKCKVSNKALPAPIEKTISKAKGQPREPQVY |
| | | | | TLPPSREEMTKNQVSLTCLVKGFYPSDIAVEWE |
| | | | | SNGQPENNYKTTPPVLDSDGSFFLYSKLTVDKS |
| | | | | RWQQGNVFSCSVMHEALHNHYTQKSLSLSPG |
| | | | | KGGGGSGGGGSGGGGSQVQLVQSGAEVKKPG |
| | | | | ASVKVSCKASGYTFTNYWMNWVRQAPGQCL |
| | | | | EWMGNIAYGVKGTNYNQKFQGRVTMTVDTSS |
| | | | | STAYMELSRLRSDDTAVYYCATRYFYVMDYW |
| | | | | GQGTLVTVSSGGGGSGGGGSGGGGSDIQMTQS |
| | | | | PSSLSASVGDRVTITCRASQDISNYLNWYQQKP |
| | | | | GKVPKLLIYYTSRLHSGVPSRFSGSGSGTDFTL |
| | | | | TISSLQPEDVATYYCVQYAQFPLTFGCGTKVEI |
| | | | | KSGGGGSEVQLVESGGGLVQPGGSLKLSCAAS |
| | | | | GFTFNKYAMNWVRQAPGKGLEWVARIRSKYN |
| | | | | NYATYYADSVKDRFTISRDDSKNTAYLQMNN |
| | | | | LKTEDTAVYYCVRHGNFGNSYISYWAYWGQG |
| | | | | TLVTVSSGGGGSGGGGSGGGGSQTVVTQEPSL |
| | | | | TVSPGGTVTLTCGSSTGAVTSGNYPNWVQQKP |
| | | | | GQAPRGLIGGTKFLAPGTPARFSGSLLGGKAAL |
| | | | | TLSGVQPEDEAEYYCVLWYSNRWVFGGGTKL |
| | | | | TVL |
| 1078. | MS 15-B12 CC x | artificial | Aa | QVQLQESGPGLVKPSETLSLTCTVSGGSISSSSY |
| | I2L x scFc x CH3 | | | FWGWIRQPPGKCLEWIGNIYYSGSSNYNPSLKS |
| | 15-E11 CC x I2L - | | | RVTISVDTSKNQFSLKLSSVTAADTAVYYCAR |
| | Full Sequence | | | LPRGDRDAFDIWGQGTMVTVSSGGGGSGGGG |
| | | | | SGGGGSDIVMTQSPSSLSASVGDRVTITCRASQ |
| | | | | GISNYLAWYQQKPGKVPKLLIYAASTLQSGVP |
| | | | | SRFSGSGSGTDFTLTISSLQPEDFATYYCQQSYS |
| | | | | TPFTFGCGTKVEIKSGGGGSEVQLVESGGGLV |
| | | | | QPGGSLKLSCAASGFTFNKYAMNWVRQAPGK |
| | | | | GMEWVARIRSKYNNYATYYADAVKDRFTISR |
| | | | | DDSKNTLYLQMNNLKTEDTAVYYCVRAGNFG |
| | | | | SSYISYFAYWGQGTLVTVSSGGGGSGGGGSGG |
| | | | | GGSQTVVTQEPSLTVSPGGTVTITCGSSTGAVT |
| | | | | SGNYPNWIQKKPGQAPRGLIGGTKFLAPGTPA |
| | | | | RFSGSLEGGKAALTLSGVQPEDEAEYYCVLYY |
| | | | | SNRWVFGSGTKLTVLGGGGSGGGGSGGGGSD |
| | | | | KTHTCPPCPAPELLGGPSVFLFPPKPKDTLMISR |
| | | | | TPEVTCVVVDVSHEDPEVKFNWYVDGVEVHN |
| | | | | AKTKPCEEQYGSTYRCVSVLTVLHQDWLNGK |
| | | | | EYKCKVSNKALPAPIEKTISKAKGQPREPQVYT |
| | | | | LPPSREEMTKNQVSLTCLVKGFYPSDIAVEWES |
| | | | | NGQPENNYKTTPPVLDSDGSFFLYSKLTVDKS |
| | | | | RWQQGNVFSCSVMHEALHNHYTQKSLSLSPG |
| | | | | KGGGGSGGGGSGGGGSGGGGSGGGGSGGGGS |
| | | | | DKTHTCPPCPAPELLGGPSVFLFPPKPKDTLMIS |
| | | | | RTPEVTCVVVDVSHEDPEVKFNWYVDGVEVH |
| | | | | NAKTKPCEEQYGSTYRCVSVLTVLHQDWLNG |
| | | | | KEYKCKVSNKALPAPIEKTISKAKGQPREPQVY |
| | | | | TLPPSREEMTKNQVSLTCLVKGFYPSDIAVEWE |
| | | | | SNGQPENNYKTTPPVLDSDGSFFLYSKLTVDKS |
| | | | | RWQQGNVFSCSVMHEALHNHYTQKSLSLSPG |
| | | | | KGGGGSGGGGSGGGGSQVQLVQSGAEVKKPG |
| | | | | ASVKVSCKASGYTFTNYWMNWVRQAPGQCL |
| | | | | EWMGNIAYGVKGTNYNQKFQGRVTMTVDTSS |
| | | | | STAYMELSRLRSDDTAVYYCATRYFYVMDYW |
| | | | | GQGTLVTVSSGGGGSGGGGSGGGGSDIQMTQS |
| | | | | PSSLSASVGDRVTITCRASQDISNYLNWYQQKP |
| | | | | GKVPKLLIYYTSRLHSGVPSRFSGSGSGTDFTL |
| | | | | TISSLQPEDVATYYCVQYAQFPLTFGCGTKVEI |
| | | | | KSGGGGSEVQLVESGGGLVQPGGSLKLSCAAS |
| | | | | GFTFNKYAMNWVRQAPGKGMEWVARIRSKY |
| | | | | NNYATYYADAVKDRFTISRDDSKNTLYLQMN |
| | | | | NLKTEDTAVYYCVRAGNFGSSYISYFAYWGQ |
| | | | | GTLVTVSSGGGGSGGGGSGGGGSQTVVTQEPS |
| | | | | LTVSPGGTVTITCGSSTGAVTSGNYPNWIQKKP |
| | | | | GQAPRGLIGGTKFLAPGTPARFSGSLEGGKAAL |
| | | | | TLSGVQPEDEAEYYCVLYYSNRWVFGSGTKLT |
| | | | | VL |
| 1079. | MS 15-B12 CC x | artificial | Aa | QVQLQESGPGLVKPSETLSLTCTVSGGSISSSSY |
| | I2M2 x scFc x | | | FWGWIRQPPGKCLEWIGNIYYSGSSNYNPSLKS |
| | CH3 15-E11 CC x | | | RVTISVDTSKNQFSLKLSSVTAADTAVYYCAR |
| | I2M2 - Full | | | LPRGDRDAFDIWGQGTMVTVSSGGGGSGGGG |
| | Sequence | | | SGGGGSDIVMTQSPSSLSASVGDRVTITCRASQ |
| | | | | GISNYLAWYQQKPGKVPKLLIYAASTLQSGVP |
| | | | | SRFSGSGSGTDFTLTISSLQPEDFATYYCQQSYS |
| | | | | TPFTFGCGTKVEIKSGGGGSEVQLVESGGGLV |
| | | | | QPGGSLKLSCAASGFTFNKYAINWVREAPGKG |
| | | | | LEWVARIRSKYNNYATYYADAVKDRFTISRDD |
| | | | | SKNTAYLQMNNLKTEDTAVYYCVRNANFGTS |
| | | | | YISYFAYWGQGTLVTVSSGGGGSGGGGSGGG |
| | | | | GSQTVVTQEPSLTVSPGGTVTLTCGSSTGAVTS |
| | | | | GNYPNWVQKKPGQAPRGLIGGTKFLAPGTPAR |
| | | | | FSGSLLGGKAALTLSGVQPEDEAEYYCVLWYS |
| | | | | NRWVFGSGTKLTVLGGGGSGGGGSGGGGSDK |
| | | | | THTCPPCPAPELLGGPSVFLFPPKPKDTLMISRT |
| | | | | PEVTCVVVDVSHEDPEVKFNWYVDGVEVHNA |
| | | | | KTKPCEEQYGSTYRCVSVLTVLHQDWLNGKE |
| | | | | YKCKVSNKALPAPIEKTISKAKGQPREPQVYTL |
| | | | | PPSREEMTKNQVSLTCLVKGFYPSDIAVEWES |
| | | | | NGQPENNYKTTPPVLDSDGSFFLYSKLTVDKS |
| | | | | RWQQGNVFSCSVMHEALHNHYTQKSLSLSPG |
| | | | | KGGGGSGGGGSGGGGSGGGGSGGGGSGGGGS |
| | | | | DKTHTCPPCPAPELLGGPSVFLFPPKPKDTLMIS |
| | | | | RTPEVTCVVVDVSHEDPEVKFNWYVDGVEVH |
| | | | | NAKTKPCEEQYGSTYRCVSVLTVLHQDWLNG |
| | | | | KEYKCKVSNKALPAPIEKTISKAKGQPREPQVY |
| | | | | TLPPSREEMTKNQVSLTCLVKGFYPSDIAVEWE |
| | | | | SNGQPENNYKTTPPVLDSDGSFFLYSKLTVDKS |
| | | | | RWQQGNVFSCSVMHEALHNHYTQKSLSLSPG |
| | | | | KGGGGSGGGGSGGGGSQVQLVQSGAEVKKPG |
| | | | | ASVKVSCKASGYTFTNYWMNWVRQAPGQCL |
| | | | | EWMGNIAYGVKGTNYNQKFQGRVTMTVDTSS |
| | | | | STAYMELSRLRSDDTAVYYCATRYFYVMDYW |
| | | | | GQGTLVTVSSGGGGSGGGGSGGGGSDIQMTQS |
| | | | | PSSLSASVGDRVTITCRASQDISNYLNWYQQKP |
| | | | | GKVPKLLIYYTSRLHSGVPSRFSGSGSGTDFTL |
| | | | | TISSLQPEDVATYYCVQYAQFPLTFGCGTKVEI |
| | | | | KSGGGGSEVQLVESGGGLVQPGGSLKLSCAAS |
| | | | | GFTFNKYAINWVREAPGKGLEWVARIRSKYN |
| | | | | NYATYYADAVKDRFTISRDDSKNTAYLQMNN |
| | | | | LKTEDTAVYYCVRNANFGTSYISYFAYWGQG |
| | | | | TLVTVSSGGGGSGGGGSGGGGSQTVVTQEPSL |
| | | | | TVSPGGTVTLTCGSSTGAVTSGNYPNWVQKKP |
| | | | | GQAPRGLIGGTKFLAPGTPARFSGSLLGGKAAL |
| | | | | TLSGVQPEDEAEYYCVLWYSNRWVFGSGTKL |
| | | | | TVL |
| 1080. | MS 15-B12 CC x | artificial | Aa | QVQLQESGPGLVKPSETLSLTCTVSGGSISSSSY |
| | I2M2 x scFc x | | | FWGWIRQPPGKCLEWIGNIYYSGSSNYNPSLKS |
| | CH3 15-E11 x I2L - | | | RVTISVDTSKNQFSLKLSSVTAADTAVYYCAR |
| | Full Sequence | | | LPRGDRDAFDIWGQGTMVTVSSGGGGSGGGG |
| | | | | SGGGGSDIVMTQSPSSLSASVGDRVTITCRASQ |
| | | | | GISNYLAWYQQKPGKVPKLLIYAASTLQSGVP |
| | | | | SRFSGSGSGTDFTLTISSLQPEDFATYYCQQSYS |
| | | | | TPFTFGCGTKVEIKSGGGGSEVQLVESGGGLV |
| | | | | QPGGSLKLSCAASGFTFNKYAINWVREAPGKG |
| | | | | LEWVARIRSKYNNYATYYADAVKDRFTISRDD |
| | | | | SKNTAYLQMNNLKTEDTAVYYCVRNANFGTS |
| | | | | YISYFAYWGQGTLVTVSSGGGGSGGGGSGGG |
| | | | | GSQTVVTQEPSLTVSPGGTVTLTCGSSTGAVTS |
| | | | | GNYPNWVQKKPGQAPRGLIGGTKFLAPGTPAR |
| | | | | FSGSLLGGKAALTLSGVQPEDEAEYYCVLWYS |
| | | | | NRWVFGSGTKLTVLGGGGSGGGGSGGGGSDK |
| | | | | THTCPPCPAPELLGGPSVFLFPPKPKDTLMISRT |
| | | | | PEVTCVVVDVSHEDPEVKFNWYVDGVEVHNA |
| | | | | KTKPCEEQYGSTYRCVSVLTVLHQDWLNGKE |
| | | | | YKCKVSNKALPAPIEKTISKAKGQPREPQVYTL |
| | | | | PPSREEMTKNQVSLTCLVKGFYPSDIAVEWES |
| | | | | NGQPENNYKTTPPVLDSDGSFFLYSKLTVDKS |
| | | | | RWQQGNVFSCSVMHEALHNHYTQKSLSLSPG |
| | | | | KGGGGSGGGGSGGGGSGGGGSGGGGSGGGGS |
| | | | | DKTHTCPPCPAPELLGGPSVFLFPPKPKDTLMIS |
| | | | | RTPEVTCVVVDVSHEDPEVKFNWYVDGVEVH |
| | | | | NAKTKPCEEQYGSTYRCVSVLTVLHQDWLNG |
| | | | | KEYKCKVSNKALPAPIEKTISKAKGQPREPQVY |
| | | | | TLPPSREEMTKNQVSLTCLVKGFYPSDIAVEWE |
| | | | | SNGQPENNYKTTPPVLDSDGSFFLYSKLTVDKS |
| | | | | RWQQGNVFSCSVMHEALHNHYTQKSLSLSPG |
| | | | | KGGGGSGGGGSGGGGSQVQLVQSGAEVKKPG |
| | | | | ASVKVSCKASGYTFTNYWMNWVRQAPGQCL |
| | | | | EWMGNIAYGVKGTNYNQKFQGRVTMTVDTSS |
| | | | | STAYMELSRLRSDDTAVYYCATRYFYVMDYW |
| | | | | GQGTLVTVSSGGGGSGGGGSGGGGSDIQMTQS |
| | | | | PSSLSASVGDRVTITCRASQDISNYLNWYQQKP |
| | | | | GKVPKLLIYYTSRLHSGVPSRFSGSGSGTDFTL |
| | | | | TISSLQPEDVATYYCVQYAQFPLTFGCGTKVEI |
| | | | | KSGGGGSEVQLVESGGGLVQPGGSLKLSCAAS |
| | | | | GFTFNKYAMNWVRQAPGKGMEWVARIRSKY |
| | | | | NNYATYYADAVKDRFTISRDDSKNTLYLQMN |
| | | | | NLKTEDTAVYYCVRAGNFGSSYISYFAYWGQ |
| | | | | GTLVTVSSGGGGSGGGGSGGGGSQTVVTQEPS |
| | | | | LTVSPGGTVTITCGSSTGAVTSGNYPNWIQKKP |
| | | | | GQAPRGLIGGTKFLAPGTPARFSGSLEGGKAAL |
| | | | | TLSGVQPEDEAEYYCVLYYSNRWVFGSGTKLT |
| | | | | VL |
| 1081. | Ubiquitin | artificial | Aa | MQIFVKTLTGKTITLEVEPSDTIENVKAKIQDKE |
| | | | | GIPPDQQRLIFAGKQLEDGRTLSDYNIQKESTL |
| | | | | HLVLRLRGG |
| 1082. | HSP70-1 | artificial | Aa | AAAIGIDLGTTYSCVGVFQHGKVEIIANDQGNR |
| | | | | TTPSYVAFTDTERLIGDAAKNQVALNPQNTVF |
| | | | | DAKRLIGRKFGDPVVQSDMKHWPFQVINDGD |
| | | | | KPKVQVSYKGETKAFYPEEISSMVLTKMKEIA |
| | | | | EAYLGYPVTNAVITVPAYFNDSQRQATKDAGV |
| | | | | IAGLNVLRIINEPTAAAIAYGLDRTGKGERNVLI |
| | | | | FDLGGGTFDVSILTIDDGIFEVKATAGDTHLGG |
| | | | | EDFDNRLVNHFVEEFKRKHKKDISQNKRAVRR |
| | | | | LRTACERAKRTLSSSTQASLEIDSLFEGIDFYTSI |
| | | | | TRARFEELCSDLFRSTLEPVEKALRDAKLDKA |
| | | | | QIHDLVLVGGSTRIPKVQKLLQDFFNGRDLNKS |
| | | | | INPDEAVAYGAAVQAAILM |
| 1083. | beta 2 | artificial | Aa | MIQRTPKIQVYSRHPAENGKSNFLNCYVSGFHP |
| | microglobulin | | | SDIEVDLLKNGERIEKVEHSDLSFSKDWSFYLL |
| | | | | YYTEFTPTEKDEYACRVNHVTLSQPKIVKWD |
| 1084. | SAND domain | artificial | Aa | DMEIAYPITCGESKAILLWKKFVCPGINVKCVK |
| | | | | FNDQLISPKHFVHLAGKSTLKDWKRAIRLGGI |
| | | | | MLRKMMDSGQIDFYQHDKVCSNTCR |
| 1085. | Green fluorescent | artificial | Aa | MSKGEELFTGVVPILVELDGDVNGHKFSVSGE |
| | protein (GFP) | | | GEGDATYGKLTLKFICTTGKLPVPWPTLVTTFT |
| | | | | YGVQCFSRYPDHMKRHDFFKSAMPEGYVQER |
| | | | | TIFFKDDGNYKTRAEVKFEGDTLVNRIELKGID |
| | | | | FKEDGNILGHKLEYNYNSHNVYIMADKQKNGI |
| | | | | KVNFKIRHNIEDGSVQLADHYQQNTPIGDGPV |
| | | | | LLPDNHYLSTQSALSKDPNEKRDHMVLLEFVT |
| | | | | AAGITHGMDELYK |
| 1086. | VHH antibody | artificial | Aa | QVQLQESGGGLVQAGDSLKLSCEASGDSIGTY |
| | lama domain | | | VIGWFRQAPGKERIYLATIGRNLVGPSDFYTRY |
| | | | | ADSVKGRFAVSRDNAKNTVNLQMNSLKPEDT |
| | | | | AVYYCAAKTTTWGGNDPNNWNYWGQGTQV |
| | | | | TVSS |
| 1087. | PSI domain from | artificial | Aa | GSAMGCRHFQSCSQCLSAPPFVQCGWCHDKC |
| | Met-receptor | | | VRSEECLSGTWTQQICL |
| 1088. | Fibronectin type | artificial | Aa | RLDAPSQIEVKDVTDTTALITWFKPLAEIDGIEL |
| | III domain from | | | TYGIKDVPGDRTTIDLTEDENQYSIGNLKPDTE |
| | tenascin | | | YEVSLISRRGDMSSNPAKETFTT |
| 1089. | Granulocyte- | artificial | Aa | APARSPSPSTQPWEHVNAIQEARRLLNLSRDTA |
| | macrophage | | | AEMNETVEVISEMFDLQEPTCLQTRLELYKQG |
| | colony-stimulating | | | LRGSLTKLKGPLTMMASHYKQHCPPTPETSCA |
| | factor (GM-CSF) | | | TQIITFESFKENLKDFLLVIPFDCWEPVQE |
| 1090. | Interleukin-4 | artificial | Aa | HKCDITLQEIIKTLNSLTEQKTLCTELTVTDIFA |
| | | | | ASKNTTEKETFCRAATVLRQFYSHHEKDTRCL |
| | | | | GATAQQFHRHKQLIRFLKRLDRNLWGLAGLNS |
| | | | | CPVKEANQSTLENFLERLKTIMREKYSKCSS |
| 1091. | CD137L | artificial | Aa | DPAGLLDLRQGMFAQLVAQNVLLIDGPLSWYS |
| | Ectodomain | | | DPGLAGVSLTGGLSYKEDTKELVVAKAGVYY |
| | | | | VFFQMELRRVVAGEGSGSVSLALHLMPLRSAA |
| | | | | GAAALALTVDLPPASSEARNSAFGFQGRLLHL |
| | | | | SAGQRLGVHLHTEARARHAWQLTQGATVLGL |
| | | | | FRVTPEIPA |
| 1092. | Interleukin-2 | artificial | Aa | APTSSSTKKTQLQLEHLLLDLQMILNGINNYKN |
| | | | | PKLTRMLTFKFYMPKKATELKHLQCLEEELKP |
| | | | | LEEVLNLAQSKNFHLRPRDLISNINVIVLELKGS |
| | | | | ETTFMCEYADETATIVEFLNRWITFAQSIISTLT |
| 1093. | PD-1 binding | artificial | Aa | AFTVTVPKDLYVVEYGSNMTIECKFPVEKELD |
| | domain from | | | LAALIVYWEMEDKNIIQFVHGEEDLKVQHSSY |
| | human | | | RQRARLLKDQLSLGNAALQITDVKLQDAGVY |
| | Programmed cell | | | RCMISYGGADYKRITVKVNAPYAAALEHHHH |
| | death 1 ligand 1 | | | |
| | (PDL1) | | | |
| 1094. | Tim-3 (AS 24- | artificial | Aa | SEYRAEVGQNAYLPCFYTPAAPGNLVPVCWG |
| | 130) | | | KGACPVFECGNVVLRTDERDVNYWTSRYWLN |
| | | | | GDFRKGDVSLTIENVTLADSGIYCCRIQIPGIMN |
| | | | | DEKFNLKLVIK |
| 1095. | MiniSOG | artificial | Aa | MEKSFVITDPRLPDNPIIFASDGFLELTEYSREEI |
| | | | | LGRNGRFLQGPETDQATVQKIRDAIRDQREITV |
| | | | | QLINYTKSGKKFWNLLHLQPMRDQKGELQYFI |
| | | | | GVQLDGEFIPNPLLG |
| 1096. | A(EAAAK)4ALE | artificial | Aa | AEAAAKEAAAKEAAAKEAAAKALEAEAAAK |
| | A(EAAAK)4A | | | EAAAKEAAAKEAAAKA |
| 1097. | (PA)25P | artificial | Aa | PAPAPAPAPAPAPAPAPAPAPAPAPAPAPAPAP |
| | | | | APAPAPAPAPAPAPAPAP |
| 1098. | SpyCatcher | artificial | Aa | VTTLSGLSGEQGPSGDMTTEEDSATHIKFSKRD |
| | | | | EDGRELAGATMELRDSSGKTISTWISDGHVKD |
| | | | | FYLYPGKYTFVETAAPDGYEVATAITFTVNEQ |
| | | | | GQVTVNGEATKGDAHT |
| 1099. | SpyTag | artificial | Aa | VPTIVMVDAYKRYK |
| 1100. | DogTag | artificial | Aa | DIPATYEFTDGKHYITNEPIPPK |
| 1101. | SnoopTagJr | artificial | Aa | KLGSIEFIKVNK |
| 1102. | MS 15-B12 CC x | artificial | Aa | QVQLQESGPGLVKPSETLSLTCTVSGGSISSSSY |
| | G4S3x heFc(A) x | | | FWGWIRQPPGKCLEWIGNIYYSGSSNYNPSLKS |
| | G4S3 x CH3 15- | | | RVTISVDTSKNQFSLKLSSVTAADTAVYYCAR |
| | E11 CC | | | LPRGDRDAFDIWGQGTMVTVSSGGGGSGGGG |
| | | | | SGGGGSDIVMTQSPSSLSASVGDRVTITCRASQ |
| | | | | GISNYLAWYQQKPGKVPKLLIYAASTLQSGVP |
| | | | | SRFSGSGSGTDFTLTISSLQPEDFATYYCQQSYS |
| | | | | TPFTFGCGTKVEIKSGGGGSGGGGSGGGGSDK |
| | | | | THTCPPCPAPELLGGPSVFLFPPKPKDTLMISRT |
| | | | | PEVTCVVVDVSHEDPEVKFNWYVDGVEVHNA |
| | | | | KTKPCEEQYGSTYRCVSVLTVLHQDWLNGKE |
| | | | | YKCKVSNKALPAPIEKTISKAKGQPREPQVYTL |
| | | | | PPSREEMTKNQVSLTCLVKGFYPSDIAVEWES |
| | | | | NGQPENNYDTTPPVLDSDGSFFLYSDLTVDKS |
| | | | | RWQQGNVFSCSVMHEALHNHYTQDSLSLSPG |
| | | | | KGGGGSGGGGSGGGGSQVQLVQSGAEVKKPG |
| | | | | ASVKVSCKASGYTFTNYWMNWVRQAPGQCL |
| | | | | EWMGNIAYGVKGTNYNQKFQGRVTMTVDTSS |
| | | | | STAYMELSRLRSDDTAVYYCATRYFYVMDYW |
| | | | | GQGTLVTVSSGGGGSGGGGSGGGGSDIQMTQS |
| | | | | PSSLSASVGDRVTITCRASQDISNYLNWYQQKP |
| | | | | GKVPKLLIYYTSRLHSGVPSRFSGSGSGTDFTL |
| | | | | TISSLQPEDVATYYCVQYAQFPLTFGCGTKVEI |
| | | | | K |
| 1103. | 6H10-09 x G4S3 x | artificial | Aa | EVQLVESGGGLVQPGGSLKLSCAASGFTFNKY |
| | heFc(B) x GS3 x | | | AMNWVRQAPGKGMEWVARIRSKYNNYATYY |
| | 6H10.09 | | | ADAVKDRFTISRDDSKNTLYLQMNNLKTEDTA |
| | | | | VYYCVRAGNFGSSYISYFAYWGQGTLVTVSSG |
| | | | | GGGSGGGGSGGGGSQTVVTQEPSLTVSPGGTV |
| | | | | TITCGSSTGAVTSGNYPNWIQKKPGQAPRGLIG |
| | | | | GTKFLAPGTPARFSGSLEGGKAALTLSGVQPED |
| | | | | EAEYYCVLYYSNRWVFGSGTKLTVLGGGGSG |
| | | | | GGGSGGGGSDKTHTCPPCPAPELLGGPSVFLFP |
| | | | | PKPKDTLMISRTPEVTCVVVDVSHEDPEVKEN |
| | | | | WYVDGVEVHNAKTKPCEEQYGSTYRCVSVLT |
| | | | | VLHQDWLNGKEYKCKVSNKALPAPIEKTISKA |
| | | | | KGQPREPQVYTLPPSRKEMTKNQVSLTCLVKG |
| | | | | FYPSDIAVEWESNGQPENNYKTTPPVLKSDGSF |
| | | | | FLYSKLTVDKSRWQQGNVFSCSVMHEALHNH |
| | | | | YTQKSLSLSPGKGGGGSGGGGSGGGGSEVQLV |
| | | | | ESGGGLVQPGGSLKLSCAASGFTFNKYAMNW |
| | | | | VRQAPGKGMEWVARIRSKYNNYATYYADAV |
| | | | | KDRFTISRDDSKNTLYLQMNNLKTEDTAVYYC |
| | | | | VRAGNFGSSYISYFAYWGQGTLVTVSSGGGGS |
| | | | | GGGGSGGGGSQTVVTQEPSLTVSPGGTVTITC |
| | | | | GSSTGAVTSGNYPNWIQKKPGQAPRGLIGGTK |
| | | | | FLAPGTPARFSGSLEGGKAALTLSGVQPEDEAE |
| | | | | YYCVLYYSNRWVFGSGTKLTVL |
| 1104. | CL1 9-G4 scFab8 | artificial | Aa | QVQLVQSGAEVKKPGASVKVSCKASGYTFTD |
| | x G4S × I2Ccc x | | | YYMHWVRQAPGQGLEWMGWINPNSGGPNYA |
| | G4 x scFc x G4 x | | | QKFQGRVTMTRDTSISTAHMELSRLRSDDTAV |
| | FL 4-E9 scFab8 | | | YYCAREKHAVAGIGFDYWGQGTLVTVSSAST |
| | xG4S xI2Ccc - | | | KGPSVFPLAPSSKSTSGGTAALGCLVKDYFPEP |
| | Full Sequence | | | VTVSWNSGALTSGVHTFPAVLQSSGLYSLSSV |
| | | | | VTVPSSSLGTQTYICNVNHKPSNTKVDKKVEP |
| | | | | KSCGGGGSGGGGSGGGGSGGGGSGGGGSGGG |
| | | | | GSGGGGSGGGGSDIQMTQSPSSVSASVGDRVTI |
| | | | | TCQASQDISNYLNWYQQKPGKAPKLLIYAASS |
| | | | | LESGVPSRFSGSGSGTDFTLTISSLQPEDFATYY |
| | | | | CQQANSFPLTFGPGTKVDIKRTVAAPSVFIFPPS |
| | | | | DEQLKSGTASVVCLLNNFYPREAKVQWKVDN |
| | | | | ALQSGNSQESVTEQDSKDSTYSLSSTLTLSKAD |
| | | | | YEKHKVYACEVTHQGLSSPVTKSFNRGECSGG |
| | | | | GGSEVQLVESGGGLVQPGGSLKLSCAASGFTF |
| | | | | NKYAMNWVRQAPGKCLEWVARIRSKYNNYA |
| | | | | TYYADSVKDRFTISRDDSKNTAYLQMNNLKTE |
| | | | | DTAVYYCVRHGNFGNSYISYWAYWGQGTLVT |
| | | | | VSSGGGGSGGGGSGGGGSQTVVTQEPSLTVSP |
| | | | | GGTVTLTCGSSTGAVTSGNYPNWVQQKPGQA |
| | | | | PRGLIGGTKFLAPGTPARFSGSLLGGKAALTLS |
| | | | | GVQPEDEAEYYCVLWYSNRWVFGCGTKLTVL |
| | | | | GGGGDKTHTCPPCPAPELLGGPSVFLFPPKPKD |
| | | | | TLMISRTPEVTCVVVDVSHEDPEVKFNWYVDG |
| | | | | VEVHNAKTKPCEEQYGSTYRCVSVLTVLHQD |
| | | | | WLNGKEYKCKVSNKALPAPIEKTISKAKGQPR |
| | | | | EPQVYTLPPSREEMTKNQVSLTCLVKGFYPSDI |
| | | | | AVEWESNGQPENNYKTTPPVLDSDGSFFLYSK |
| | | | | LTVDKSRWQQGNVFSCSVMHEALHNHYTQKS |
| | | | | LSLSPGKGGGGSGGGGSGGGGSGGGGSGGGG |
| | | | | SGGGGSDKTHTCPPCPAPELLGGPSVFLFPPKP |
| | | | | KDTLMISRTPEVTCVVVDVSHEDPEVKFNWYV |
| | | | | DGVEVHNAKTKPCEEQYGSTYRCVSVLTVLH |
| | | | | QDWLNGKEYKCKVSNKALPAPIEKTISKAKGQ |
| | | | | PREPQVYTLPPSREEMTKNQVSLTCLVKGFYPS |
| | | | | DIAVEWESNGQPENNYKTTPPVLDSDGSFFLYS |
| | | | | KLTVDKSRWQQGNVFSCSVMHEALHNHYTQK |
| | | | | SLSLSPGKGGGGQVTLKESGPTLVKPTETLTLT |
| | | | | CTVSGFSFRNARMGVSWIRQPPGKALEWLAHI |
| | | | | FSNDEKSYSTSLKSRLTISKDTSKSQVVLTMTN |
| | | | | MDPVDTATYFCARVPEYSSGWYRFDYWGQGT |
| | | | | LVTVSSASTKGPSVFPLAPSSKSTSGGTAALGC |
| | | | | LVKDYFPEPVTVSWNSGALTSGVHTFPAVLQS |
| | | | | SGLYSLSSVVTVPSSSLGTQTYICNVNHKPSNT |
| | | | | KVDKKVEPKSCGGGGSGGGGSGGGGSGGGGS |
| | | | | GGGGSGGGGSGGGGSGGGGSDIQMTQSPSSLS |
| | | | | ASVGDRVTISCRASQSIRSYLNWYQQKPGKAP |
| | | | | KLLIYATSSLQGGVPSRFSGSGSGTDFTLTISSL |
| | | | | QPEDFATYYCQQSYSTPFTFGPGTKVEIKRTVA |
| | | | | APSVFIFPPSDEQLKSGTASVVCLLNNFYPREA |
| | | | | KVQWKVDNALQSGNSQESVTEQDSKDSTYSL |
| | | | | SSTLTLSKADYEKHKVYACEVTHQGLSSPVTK |
| | | | | SFNRGECSGGGGSEVQLVESGGGLVQPGGSLK |
| | | | | LSCAASGFTFNKYAMNWVRQAPGKCLEWVA |
| | | | | RIRSKYNNYATYYADSVKDRFTISRDDSKNTA |
| | | | | YLQMNNLKTEDTAVYYCVRHGNFGNSYISYW |
| | | | | AYWGQGTLVTVSSGGGGSGGGGSGGGGSQTV |
| | | | | VTQEPSLTVSPGGTVTLTCGSSTGAVTSGNYPN |
| | | | | WVQQKPGQAPRGLIGGTKFLAPGTPARFSGSL |
| | | | | LGGKAALTLSGVQPEDEAEYYCVLWYSNRWV |
| | | | | FGCGTKLTVL |
| 1105. | CL1 9-G4 scFab8 - | artificial | Aa | DYYMH |
| | HCDR1 | | | |
| 1106. | CL1 9-G4 scFab8 - | artificial | Aa | WINPNSGGPNYAQKFQG |
| | HCDR2 | | | |
| 1107. | CL1 9-G4 scFab8 - | artificial | Aa | EKHAVAGIGFDY |
| | HCDR3 | | | |
| 1108. | CL1 9-G4 scFab8 - | artificial | Aa | QASQDISNYLN |
| | LCDR1 | | | |
| 1109. | CL1 9-G4 scFab8 - | artificial | Aa | AASSLES |
| | LCDR2 | | | |
| 1110. | CL1 9-G4 scFab8 - | artificial | Aa | QQANSFPLT |
| | LCDR3 | | | |
| 1111. | CL1 9-G4 scFab8 - | artificial | Aa | QVQLVQSGAEVKKPGASVKVSCKASGYTFTD |
| | VH | | | YYMHWVRQAPGQGLEWMGWINPNSGGPNYA |
| | | | | QKFQGRVTMTRDTSISTAHMELSRLRSDDTAV |
| | | | | YYCAREKHAVAGIGFDYWGQGTLVTVSS |
| 1112. | CL1 9-G4 scFab8 - | artificial | Aa | DIQMTQSPSSVSASVGDRVTITCQASQDISNYL |
| | VL | | | NWYQQKPGKAPKLLIYAASSLESGVPSRFSGS |
| | | | | GSGTDFTLTISSLQPEDFATYYCQQANSFPLTFG |
| | | | | PGTKVDIK |
| 1113. | CL1 9-G4 scFab8 - | artificial | Aa | ASTKGPSVFPLAPSSKSTSGGTAALGCLVKDYF |
| | CH1 | | | PEPVTVSWNSGALTSGVHTFPAVLQSSGLYSLS |
| | | | | SVVTVPSSSLGTQTYICNVNHKPSNTKVDKKV |
| | | | | EPKSC |
| 1114. | CL1 9-G4 scFab8 - | artificial | Aa | RTVAAPSVFIFPPSDEQLKSGTASVVCLLNNFY |
| | CLK | | | PREAKVQWKVDNALQSGNSQESVTEQDSKDS |
| | | | | TYSLSSTLTLSKADYEKHKVYACEVTHQGLSS |
| | | | | PVTKSFNRGEC |
| 1115. | FL 4-E9 scFab8 - | artificial | Aa | NARMGVS |
| | HCDR1 | | | |
| 1116. | FL 4-E9 scFab8 - | artificial | Aa | HIFSNDEKSYSTSLKS |
| | HCDR2 | | | |
| 1117. | FL 4-E9 scFab8 - | artificial | Aa | VPEYSSGWYRFDY |
| | HCDR3 | | | |
| 1118. | FL 4-E9 scFab8 - | artificial | Aa | RASQSIRSYLN |
| | LCDR1 | | | |
| 1119. | FL 4-E9 scFab8 - | artificial | Aa | ATSSLQG |
| | LCDR2 | | | |
| 1120. | FL 4-E9 scFab8 - | artificial | Aa | QQSYSTPFT |
| | LCDR3 | | | |
| 1121. | FL 4-E9 scFab8 - | artificial | Aa | QVTLKESGPTLVKPTETLTLTCTVSGFSFRNAR |
| | VH | | | MGVSWIRQPPGKALEWLAHIFSNDEKSYSTSL |
| | | | | KSRLTISKDTSKSQVVLTMTNMDPVDTATYFC |
| | | | | ARVPEYSSGWYRFDYWGQGTLVTVSS |
| 1122. | FL 4-E9 scFab8 - | artificial | Aa | DIQMTQSPSSLSASVGDRVTISCRASQSIRSYLN |
| | VL | | | WYQQKPGKAPKLLIYATSSLQGGVPSRFSGSG |
| | | | | SGTDFTLTISSLQPEDFATYYCQQSYSTPFTFGP |
| | | | | GTKVEIK |
| 1123. | FL 4-E9 scFab8 - | artificial | Aa | ASTKGPSVFPLAPSSKSTSGGTAALGCLVKDYF |
| | CH1 | | | PEPVTVSWNSGALTSGVHTFPAVLQSSGLYSLS |
| | | | | SVVTVPSSSLGTQTYICNVNHKPSNTKVDKKV |
| | | | | EPKSC |
| 1124. | FL 4-E9 scFab8 - | artificial | Aa | RTVAAPSVFIFPPSDEQLKSGTASVVCLLNNFY |
| | CLK | | | PREAKVQWKVDNALQSGNSQESVTEQDSKDS |
| | | | | TYSLSSTLTLSKADYEKHKVYACEVTHQGLSS |
| | | | | PVTKSFNRGEC |
| 1125. | (G4S)8 - Linker | artificial | Aa | GGGGSGGGGSGGGGSGGGGSGGGGSGGGGSG |
| | | | | GGGSGGGGS |
| 1126. | CD3 F2B - | artificial | Aa | DYAMH |
| | HCDR1 | | | |
| 1127. | CD3 F2B - | artificial | Aa | GISWNSGSIGYADSVKG |
| | HCDR2 | | | |
| 1128. | CD3 F2B - | artificial | Aa | DSRGYGDYRLGGAY |
| | HCDR3 | | | |
| 1129. | CD3 F2B-LCDR1 | artificial | Aa | RASQSVSSNLA |
| 1130. | CD3 F2B-LCDR2 | artificial | Aa | GASTRAT |
| 1131. | CD3 F2B-LCDR3 | artificial | Aa | QQYNNWPWT |
| 1132. | CD3 F2B-VH | artificial | Aa | EVQLVESGGGLVQPGRSLRLSCAASGFTEDDY |
| | | | | AMHWVRQAPGKGLEWVSGISWNSGSIGYADS |
| | | | | VKGRFTISRDNAKNSLYLQMNSLRAEDTALYY |
| | | | | CAKDSRGYGDYRLGGAYWGQGTLVTVSS |
| 1133. | CD3 F2B- VL | artificial | Aa | EIVMTQSPATLSVSPGERATLSCRASQSVSSNL |
| | | | | AWYQQKPGQAPRLLIYGASTRATGIPARFSGS |
| | | | | GSGTEFTLTISSLQSEDFAVYYCQQYNNWPWT |
| | | | | FGQGTKVEIK |
| 1134. | CD3 F2B scFab8 - | artificial | Aa | ASTKGPSVFPLAPSSKSTSGGTAALGCLVKDYF |
| | CH1 | | | PEPVTVSWNSGALTSGVHTFPAVLQSSGLYSLS |
| | | | | SVVTVPSSSLGTQTYICNVNHKPSNTKVDKKV |
| | | | | EPKSC |
| 1135. | CD3 F2B scFab8 - | artificial | Aa | RTVAAPSVFIFPPSDEQLKSGTASVVCLLNNFY |
| | CLK | | | PREAKVQWKVDNALQSGNSQESVTEQDSKDS |
| | | | | TYSLSSTLTLSKADYEKHKVYACEVTHQGLSS |
| | | | | PVTKSFNRGEC |
| 1136. | CD3 FJan - | artificial | Aa | TYAMN |
| | HCDR1 | | | |
| 1137. | CD3 FJan - | artificial | Aa | RIRSKYNNYATYYAASVKK |
| | HCDR2 | | | |
| 1138. | CD3 FJan - | artificial | Aa | HGNFGNSYVSWFAY |
| | HCDR3 | | | |
| 1139. | CD3 FJan-LCDR1 | artificial | Aa | RSSTGAVTTSNYAN |
| 1140. | CD3 FJan-LCDR2 | artificial | Aa | GTNKRAP |
| 1141. | CD3 FJan-LCDR3 | artificial | Aa | ALWYSNLWV |
| 1142. | CD3 FCel - | artificial | Aa | TYAMN |
| | HCDR1 | | | |
| 1143. | CD3 FCel - | artificial | Aa | RIRSKYNNYATYYADSVKG |
| | HCDR2 | | | |
| 1144. | CD3 FCel - | artificial | Aa | HGNFGNSYVSWFAY |
| | HCDR3 | | | |
| 1145. | CD3 FCel-LCDR1 | artificial | Aa | GSSTGAVTSNYAN |
| 1146. | CD3 FCel-LCDR2 | artificial | Aa | GTNKRAP |
| 1147. | CD3 FCel-LCDR3 | artificial | Aa | ALWYSNLWV |
| 1148. | CD3 FHar - | artificial | Aa | KYAIN |
| | HCDR1 | | | |
| 1149. | CD3 FHar - | artificial | Aa | RIRSKYNNYATYYAQVKD |
| | HCDR2 | | | |
| 1150. | CD3 FHar - | artificial | Aa | HANFGNSYISYWAY |
| | HCDR3 | | | |
| 1151. | CD3 FHar- | artificial | Aa | ASSTGAVTSGNYPN |
| | LCDR1 | | | |
| 1152. | CD3 FHar- | artificial | Aa | GTKFLVP |
| | LCDR2 | | | |
| 1153. | CD3 FHar- | artificial | Aa | TLWYSNRWV |
| | LCDR3 | | | |
| 1154. | CDH3 HCDR1 | artificial | Aa | XXXXX |
| | consencus | | | |
| 1155. | CDH3 HCDR2 | artificial | Aa | XIXXXXXXTXYXXXXXG |
| | consencus | | | |
| 1156. | CDH3 HCDR3 | artificial | Aa | SRGVYDXXXXXXXYXMDX |
| | consencus | | | |
| 1157. | CDH3 VH | artificial | Aa | XVQLXXSGXXXXXPGXSXXXSCXASGXXFXX |
| | consensus | | | XXXXWVRQXPGXCLEWXXXIXXXXXTXYXX |
| | | | | XXXGRXTXXXDXSXXTXYXXXXXLXXXDXA |
| | | | | VYYCAXXRGVYDFKXXXALXXXDXWGQGTX |
| | | | | VTVSS |
| 1158. | CDH3 LCDR1 | artificial | Aa | XXSXXXLYSSNQXXYXX |
| | consencus | | | |
| 1159. | CDH3 LCDR2 | artificial | Aa | XXXXXXX |
| | consencus | | | |
| 1160. | CDH3 LCDR3 | artificial | Aa | XXXXXXPXT |
| | consencus | | | |
| 1161. | CDH3 VL | artificial | Aa | XIXXTQSPXXLXXSXGXRXTXXCXXSXXXLY |
| | consensus | | | NQXXYXXWYQQKPGXXPXLLXYXXXXXXXG |
| | | | | VPXRFSGSGSGTXFTLXISXLQXEDXXXYYCX |
| | | | | XXXXXPXTFGCGTKXXIK |
| 1162. | MSLN HCDR1 | artificial | Aa | SSXYXXX |
| | consencus | | | |
| 1163. | MSLN HCDR2 | artificial | Aa | XIXXXXXXXXXXXXXXX |
| | consencus | | | |
| 1164. | MSLN HCDR3 | artificial | Aa | XXXXXGXXSYXPXXXXXXXDX |
| | consencus | | | |
| 1165. | MSLN VH | artificial | Aa | XVQLXXSGXXXXXPXXXXXXXCXXSGGSXX |
| | consensus | | | XXXYXXXWXRQXPGXXLEWXXXIXXXXXXX |
| | | | | XYXXXXXXRXTXXXDXXXXXXXXXXXXXX |
| | | | | XXDTAVYYCAXXXXXXXXXSYYPXYYXXXX |
| | | | | DXWGQGTXVTVSS |
| 1166. | MSLN LCDR1 | artificial | Aa | RXXXXXXXXXXX |
| | consencus | | | |
| 1167. | MSLN LCDR2 | artificial | Aa | XXXXXXX |
| | consencus | | | |
| 1168. | MSLN LCDR3 | artificial | Aa | QXXXXXXIXX |
| | consencus | | | |
| 1169. | MSLN VL | artificial | Aa | XXXXTQXPXXXSXSXG1XXXXXCRXXXXXXX |
| | consensus | | | XXXXWYQQKPGXXPXLXIYXXXXXXXGXPX |
| | | | | RFSGSXSGXXXTLTISXXXXXDXAXYYCQXXX |
| | | | | XXXIXXFGXGTKXXXX |
| 1170. | Human CDH3 | human | Aa | MGLPRGPLASLLLLQVCWLQCAASEPCRAVFR |
| | | | | EAEVTLEAGGAEQEPGQALGKVFMGCPGQEP |
| | | | | ALFSTDNDDFTVRNGETVQERRSLKERNPLKIF |
| | | | | PSKRILRRHKRDWVVAPISVPENGKGPFPQRLN |
| | | | | QLKSNKDRDTKIFYSITGPGADSPPEGVFAVEK |
| | | | | ETGWLLLNKPLDREEIAKYELFGHAVSENGAS |
| | | | | VEDPMNISIIVTDQNDHKPKFTQDTFRGSVLEG |
| | | | | VLPGTSVMQVTATDEDDAIYTYNGVVAYSIHS |
| | | | | QEPKDPHDLMFTIHRSTGTISVISSGLDREKVPE |
| | | | | YTLTIQATDMDGDGSTTTAVAVVEILDANDNA |
| | | | | PMFDPQKYEAHVPENAVGHEVQRLTVTDLDA |
| | | | | PNSPAWRATYLIMGGDDGDHFTITTHPESNQGI |
| | | | | LTTRKGLDFEAKNQHTLYVEVTNEAPFVLKLP |
| | | | | TSTATIVVHVEDVNEAPVFVPPSKVVEVQEGIP |
| | | | | TGEPVCVYTAEDPDKENQKISYRILRDPAGWL |
| | | | | AMDPDSGQVTAVGTLDREDEQFVRNNIYEVM |
| | | | | VLAMDNGSPPTTGTGTLLLTLIDVNDHGPVPEP |
| | | | | RQITICNQSPVRQVLNITDKDLSPHTSPFQAQLT |
| | | | | DDSDIYWTAEVNEEGDTVVLSLKKFLKQDTYD |
| | | | | VHLSLSDHGNKEQLTVIRATVCDCHGHVETCP |
| | | | | GPWKGGFILPVLGAVLALLFLLLVLLLLVRKK |
| | | | | RKIKEPLLLPEDDTRDNVFYYGEEGGGEEDQD |
| | | | | YDITQLHRGLEARPEVVLRNDVAPTIIPTPMYR |
| | | | | PRPANPDEIGNFIIENLKAANTDPTAPPYDTLLV |
| | | | | FDYEGSGSDAASLSSLTSSASDQDQDYDYLNE |
| | | | | WGSRFKKLADMYGGGEDD |
| 1171. | Human CDH3 | human | Aa | VAYSIHSQEPKDPHDLMFTIHRSTGTISVISSGL |
| | epitope cluster | | | DREK |
| | D2B | | | |
| 1172. | Human CDH3 | human | Aa | VPEYTLTIQATDMDGDGSTTTAVAVVEILDAN |
| | epitope cluster | | | DNAPM |
| | D2C | | | |
| 1173. | Human CDH3 | human | Aa | FDPQKYEAHVPENAVGHEVQRLTVTDLDAPNS |
| | epitope cluster | | | PAWR |
| | D3A | | | |
| 1174. | Human CDH3 | human | Aa | YRILRDPAGWLAMDPDSGQVTAVGTLDREDE |
| | epitope cluster | | | QFVRN |
| | D4B | | | |
| 1175. | Human MSLN | human | Aa | EVEKTACPSGKKAREIDESLIFYKKWELEACVD |
| | epitope cluster E1 | | | AALLATQMDRVNAIPFTY |
| 1176. | Human MSLN | human | Aa | EQLDVLKHKLDELYPQGYPESVIQHLGYLFLK |
| | epitope cluster E2 | | | MSPEDI |
| 1177. | Human MSLN | human | Aa | RKWNVTSLETLKALLEVNKGHEMSPQVATLID |
| | epitope cluster E3 | | | RFVKGRGQLDKDTLDTLTAFYPGYLCSLSPEEL |
| | | | | SSVP |
| 1178. | Human MSLN | human | Aa | PSSIWAVRPQDLDTCDPRQLDVLYPKARLAFQ |
| | epitope cluster E4 | | | NMNGSEYFVKIQSFLG |
| 1179. | Human MSLN | human | Aa | GAPTEDLKALSQQNVSMDLATFMKLRTDAVL |
| | epitope cluster E5 | | | PLTVAEVQKLLGP |
| 1180. | Human MSLN v1 | human | Aa | MALPTARPLLGSCGTPALGSLLFLLFSLGWVQP |
| | NM_005823 | | | SRTLAGETGQEAAPLDGVLANPPNISSLSPRQL |
| | | | | LGFPCAEVSGLSTERVRELAVALAQKNVKLST |
| | | | | EQLRCLAHRLSEPPEDLDALPLDLLLFLNPDAF |
| | | | | SGPQACTRFFSRITKANVDLLPRGAPERQRLLP |
| | | | | AALACWGVRGSLLSEADVRALGGLACDLPGR |
| | | | | FVAESAEVLLPRLVSCPGPLDQDQQEAARAAL |
| | | | | QGGGPPYGPPSTWSVSTMDALRGLLPVLGQPII |
| | | | | RSIPQGIVAAWRQRSSRDPSWRQPERTILRPRF |
| | | | | RREVEKTACPSGKKAREIDESLIFYKKWELEAC |
| | | | | VDAALLATQMDRVNAIPFTYEQLDVLKHKLD |
| | | | | ELYPQGYPESVIQHLGYLFLKMSPEDIRKWNV |
| | | | | TSLETLKALLEVNKGHEMSPQVATLIDRFVKG |
| | | | | RGQLDKDTLDTLTAFYPGYLCSLSPEELSSVPP |
| | | | | SSIWAVRPQDLDTCDPRQLDVLYPKARLAFQN |
| | | | | MNGSEYFVKIQSFLGGAPTEDLKALSQQNVSM |
| | | | | DLATFMKLRTDAVLPLTVAEVQKLLGPHVEGL |
| | | | | KAEERHRPVRDWILRQRQDDLDTLGLGLQGGI |
| | | | | PNGYLVLDLSMQEALSGTPCLLGPGPVLTVLA |
| | | | | LLLASTLA |
| 1181. | Human MSLN v2 | human | Aa | MALPTARPLLGSCGTPALGSLLFLLFSLGWVQP |
| | NM_013404 | | | SRTLAGETGQEAAPLDGVLANPPNISSLSPRQL |
| | | | | LGFPCAEVSGLSTERVRELAVALAQKNVKLST |
| | | | | EQLRCLAHRLSEPPEDLDALPLDLLLFLNPDAF |
| | | | | SGPQACTRFFSRITKANVDLLPRGAPERQRLLP |
| | | | | AALACWGVRGSLLSEADVRALGGLACDLPGR |
| | | | | FVAESAEVLLPRLVSCPGPLDQDQQEAARAAL |
| | | | | QGGGPPYGPPSTWSVSTMDALRGLLPVLGQPII |
| | | | | RSIPQGIVAAWRQRSSRDPSWRQPERTILRPRF |
| | | | | RREVEKTACPSGKKAREIDESLIFYKKWELEAC |
| | | | | VDAALLATQMDRVNAIPFTYEQLDVLKHKLD |
| | | | | ELYPQGYPESVIQHLGYLFLKMSPEDIRKWNV |
| | | | | TSLETLKALLEVNKGHEMSPQAPRRPLPQVAT |
| | | | | LIDRFVKGRGQLDKDTLDTLTAFYPGYLCSLSP |
| | | | | EELSSVPPSSIWAVRPQDLDTCDPRQLDVLYPK |
| | | | | ARLAFQNMNGSEYFVKIQSFLGGAPTEDLKAL |
| | | | | SQQNVSMDLATFMKLRTDAVLPLTVAEVQKL |
| | | | | LGPHVEGLKAEERHRPVRDWILRQRQDDLDTL |
| | | | | GLGLQGGIPNGYLVLDLSMQEALSGTPCLLGP |
| | | | | GPVLTVLALLLASTLA |
| 1182. | Human MSLN v6 | human | Aa | MALPTARPLLGSCGTPALGSLLFLLFSLGWVQP |
| | AY743922 | | | SRTLAGETGQEAAPLDGVLANPPNISSLSPRQL |
| | | | | LGFPCAEVSGLSTERVRELAVALAQKNVKLST |
| | | | | EQLRCLAHRLSEPPEDLDALPLDLLLFLNPDAF |
| | | | | SGPQACTHFFSRITKANVDLLPRGAPERQRLLP |
| | | | | AALACWGVRGSLLSEADVRALGGLACDLPGR |
| | | | | FVAESAEVLLPRLVSCPGPLDQDQQEAARAAL |
| | | | | QGGGPPYGPPSTWSVSTMDALRGLLPVLGQPII |
| | | | | RSIPQGIVAAWRQRSSRDPSWRQPERTILRPRF |
| | | | | RREVEKTACPSGKKAREIDESLIFYKKWELEAC |
| | | | | VDAALLATQMDRVNAIPFTYEQLDVLKHKLD |
| | | | | ELYPQGYPESVIQHLGYLFLKMSPEDIRKWNV |
| | | | | TSLETLKALLEVNKGHEMSPQVATLIDRFVKG |
| | | | | RGQLDKDTLDTLTAFYPGYLCSLSPEELSSVPP |
| | | | | SSIWAVRPQDLDTCDPRQLDVLYPKARLAFQN |
| | | | | MNGSEYFVKIQSFLGGAPTEDLKALSQQNVSM |
| | | | | DLATFMKLRTDAVLPLTVAEVQKLLGPHVEGL |
| | | | | KAEERHRPVRDWILRQRQDDLDTLGLGLQGGI |
| | | | | PNGYLVLDLSVQEALSGTPCLLGPGPVLTVLAL |
| | | | | LLASTLA |
| 1183. | MSLN 5F11 xI2C- | artificial | aa | QVQLVESGGGLVKPGGSLRLSCAASGFTFSDY |
| | scFc | | | YMTWIRQAPGKGLEWLSYISSSGSTIYYADSV |
| | _HLE_bispecific | | | KGRFTISRDNAKNSLFLQMNSLRAEDTAVYYC |
| | molecule | | | ARDRNSHFDYWGQGTLVTVSSGGGGSGGGGS |
| | | | | GGGGSDIQMTQSPSSVSASVGDRVTITCRASQG |
| | | | | INTWLAWYQQKPGKAPKLLIYGASGLQSGVPS |
| | | | | RFSGSGSGTDFTLTISSLQPEDFATYYCQQAKSF |
| | | | | PRTFGQGTKVEIKSGGGGSEVQLVESGGGLVQ |
| | | | | PGGSLKLSCAASGFTFNKYAMNWVRQAPGKG |
| | | | | LEWVARIRSKYNNYATYYADSVKDRFTISRDD |
| | | | | SKNTAYLQMNNLKTEDTAVYYCVRHGNFGNS |
| | | | | YISYWAYWGQGTLVTVSSGGGGSGGGGSGGG |
| | | | | GSQTVVTQEPSLTVSPGGTVTLTCGSSTGAVTS |
| | | | | GNYPNWVQQKPGQAPRGLIGGTKFLAPGTPAR |
| | | | | FSGSLLGGKAALTLSGVQPEDEAEYYCVLWYS |
| | | | | NRWVFGGGTKLTVLGGGGDKTHTCPPCPAPEL |
| | | | | LGGPSVFLFPPKPKDTLMISRTPEVTCVVVDVS |
| | | | | HEDPEVKFNWYVDGVEVHNAKTKPCEEQYGS |
| | | | | TYRCVSVLTVLHQDWLNGKEYKCKVSNKALP |
| | | | | APIEKTISKAKGQPREPQVYTLPPSREEMTKNQ |
| | | | | VSLTCLVKGFYPSDIAVEWESNGQPENNYKTT |
| | | | | PPVLDSDGSFFLYSKLTVDKSRWQQGNVFSCS |
| | | | | VMHEALHNHYTQKSLSLSPGKGGGGSGGGGS |
| | | | | GGGGSGGGGSGGGGSGGGGSDKTHTCPPCPAP |
| | | | | ELLGGPSVFLFPPKPKDTLMISRTPEVTCVVVD |
| | | | | VSHEDPEVKFNWYVDGVEVHNAKTKPCEEQY |
| | | | | GSTYRCVSVLTVLHQDWLNGKEYKCKVSNKA |
| | | | | LPAPIEKTISKAKGQPREPQVYTLPPSREEMTK |
| | | | | NQVSLTCLVKGFYPSDIAVEWESNGQPENNYK |
| | | | | TTPPVLDSDGSFFLYSKLTVDKSRWQQGNVFS |
| | | | | CSVMHEALHNHYTQKSLSLSPGK |

1. An antigen-binding molecule comprising at least one polypeptide chain, wherein the molecule comprises
(i) a first binding domain, which specifically binds to a first target cell surface antigen (e.g. TAA1),
(ii) a second binding domain, which specifically binds to an extracellular epitope of the human and/or the Macaca CD3ε chain,
(iii) a third binding domain, which specifically binds to a second target cell surface antigen (e.g. TAA2), and
(iv) a fourth binding domain, which—specifically binds to an extracellular epitope of the human and/or the Macaca CD3ε chain,
wherein the first binding domain and the second binding domain form a first bispecific entity and the third and the fourth binding domain form a second bispecific entity, and
wherein the molecule comprises a spacer entity having a molecular weight of at least about 5 kDa and/or having a length of at more than 50 amino acids,
wherein the spacer entity spaces apart the first and the second bispecific entity by at least a distance of about 50 Å,
wherein the indicated distance is understood as the distance between centers of mass of the first and the second bispecific entity,
wherein the spacer entity is a globular protein, and comprises an amino acid sequence selected the group consisting of: ubiquitin (SEQ ID NO: 1081), beta 2 microglobulin (SEQ ID NO: 1083), SAND domain (SEQ ID NO: 1084), VHH antibody lama domain (SEQ ID NO: 1086), PD-1 binding domain from human Programmed cell death 1 ligand 1 (PDL1) (SEQ ID NO: 1093), a programmed cell death protein 1 (PD1) domain (SEQ ID NO: 16), human serum albumin (HSA) (SEQ ID NO: 15), single chain Fc (scFc) (SEQ ID NO: 25, 26, 27, 28, 29, 30, 31, 32, or 33), and double scFc (SEQ ID NO: 30),
wherein the spacer entity is positioned between the first and the second bispecific entity,
wherein the first binding domain is capable of binding to the first target cell surface antigen and the third binding domain is capable of binding to the second target cell surface antigen simultaneously,
wherein the first target cell surface antigen and the second target cell surface antigen are on the same target cell, and
wherein the first target cell surface antigen and the second target cell surface antigen are not identical.

2. The antigen-binding molecule of claim 1 which is a multitargeting bispecific antigen-binding molecule.

3. The antigen-binding molecule of claim 2, wherein the arrangement of domains in an amino to carboxyl order is selected from the group consisting of
(i) first and second domain, spacer, third and fourth domain,
(ii) first and second domain, spacer, fourth and third domain,
(iii) second and the first domain, spacer, third and fourth domain, and
(iv) second and first domain, spacer, fourth and third domain.

4. The antigen-binding molecule of claim 1, wherein said spacer entity has a molecular weight of at least 10 kDa, and/or wherein said spacer entity comprises an amino acid sequence which comprises more than 50 amino acids.

5. (canceled)

6. The antigen-binding molecule of claim 1, wherein the spacer entity is a globular protein, wherein the distance between the C alpha atoms of the first amino acid located at the N-terminus and the last amino acid at the C-terminus are spaced apart by at least 20 Å, in order to effectively space apart the first and the second bispecific entity.

7-10. (canceled)

11. The antigen-binding molecule of claim 1, wherein the molecule is a single polypeptide chain.

12. (canceled)

13. The antigen-binding molecule of claim 1, wherein the molecule comprises two polypeptide chains.

14. An antigen-binding molecule comprising two polypeptide chains, wherein
(i) the first polypeptide chain comprises a first binding domain which specifically binds to a first target cell surface antigen, a second binding domain which specifically binds to an extracellular epitope of the human and/or the Macaca CD3ε chain, and a first polypeptide monomer comprising a hinge, a CH2 domain and a CH3 domain, and
(ii) the second polypeptide chain comprises a third binding domain which specifically binds to a second target cell surface antigen, a fourth binding domain which specifically binds to an extracellular epitope of the human and/or the Macaca CD3ε chain, and a second polypeptide monomer comprising a hinge, a CH2 domain and a CH3 domain,
wherein the two polypeptide monomers form a heterodimer pairing the CH2 and the CH3 domains of the two peptide monomers, respectively, wherein the CH2 domain of the first peptide monomer is linked to the first or second domain of the first bispecific entity in C-terminal position of said entity, and wherein the CH3 domain of the second peptide monomer is linked to the third or fourth domain of the second bispecific entity in N-terminal position of said entity, i.e. the N-terminus of the second polypeptide chain is at the CH2 domain of the second polypeptide monomer and the C-terminus is at the third or fourth domain,
wherein the first and second polypeptide monomer form a heterodimer, thereby connecting the first and the second polypeptide chain, wherein the first binding domain is capable of binding to the first target cell surface antigen and the third binding domain is capable of binding to the second target cell surface antigen simultaneously, wherein the first target cell surface antigen and the second target cell surface antigen are on the same target cell.

15. The antigen-binding molecule of claim 14, wherein the first peptide monomer of the first peptide chain is SEQ ID NO 35 and the second peptide monomer of the second peptide chain is SEQ ID NO 36, and wherein the two peptide monomers form a heterodimer.

16. The antigen-binding molecule of claim 1, wherein
(i) the first and third domains comprise two antibody-derived variable domains and the second and the fourth domains comprises two antibody-derived variable domains;
(ii) the first and third domains comprise one antibody-derived variable domain and the second and the fourth domains comprises two antibody-derived variable domains;
(iii) the first and third domains comprise two antibody-derived variable domains and the second and the fourth domains comprise one antibody-derived variable domain; or
(iv) the first domain comprises one antibody-derived variable domain and the third domain comprises one antibody-derived variable domain.

17. The antigen-binding molecule of claim 14, wherein the antigen-binding molecule comprises two polypeptide chains, wherein
the first polypeptide chain comprises a VH of the first domain, a VH second domain, the first polypeptide monomer comprising a hinge, a CH2 and a CH3 domain, a VH of the third domain, and a VH of the fourth domain; and
the second polypeptide chain comprises a VL of the first domain, a VL second domain, the first polypeptide monomer comprising a hinge, a CH2 and a CH3 domain, a VL of the third domain, and a VL of the fourth domain,
wherein the first and second polypeptide monomers form a heterodimer, thereby connecting the first and the second polypeptide chain.

18. The antigen-binding molecule of claim 1, wherein the antigen binding molecule, wherein the first, second, third and fourth binding domains each comprise in an amino to carboxyl order a VH domain and a VL domain,
wherein the VH and VL within each domain is connected by a peptide linker, which is a flexible linker which comprises serine, glutamine and/or glycine as amino acid building blocks.

19. The antigen-binding molecule of claim 1, wherein the peptide linker comprises or consists of S(G4X)n and (G4X)n, wherein X is selected from the group consisting of Q, T, N, C, G, A, V, I, L, and M, and wherein n is an integer selected from integers 1 to 20, preferably wherein X is Q, wherein preferably the peptide linker is (G4X)n, and X is Q.

20. The antigen-binding molecule of claim 1, wherein the peptide linker between the first binding domain and the second binding domain and the third binding domain and the fourth binding domain is preferably a flexible linker which comprises serine, glutamine and/or glycine or glutamic acid, alanine and lysine as amino acid building blocks.

21. The antigen-binding molecule of claim 1, wherein the peptide linker between the first binding domain or the second binding domain and the spacer, and/or the third binding domain and the fourth binding domain and the spacer, respectively, is a short linker rich in small and/or hydrophilic amino acids.

22. The antigen-binding molecule of claim 1, wherein any of the first target cell surface antigen and the second target cell surface antigen is selected from the group consisting of CS1, BCMA, CDH3, FLT3, CD123, CD20, CD22, EpCAM, MSLN and CLL1.

23-25. (canceled)

26. The antigen-binding molecule of claim 1, wherein the first target cell surface antigen and the second target cell surface antigen, respectively, are selected from the group consisting of CS1 and BCMA, BCMA and CS1, FLT3 and CD123, CD123 and FLT3, CD20 and CD22, CD22 and CD20, EpCAM and MSLN, MSLN and EpCAM, MSLN and CDH3, CDH3 and MSLN, FLT3 and CLL1, and CLL1 and FLT3.

27. The antigen-binding molecule of claim 1, wherein the first target cell surface antigen and/or the second target cell surface antigen is human MSLN (selected from SEQ ID NOs: 1181, 1182 and 1183), and wherein the first and/or third binding domain of the antigen-binding molecule of the invention binds to human MSLN epitope cluster E1 (SEQ ID NO: 1175, aa 296-346 position according to Kabat) as determined by murine chimere sequence analysis as described herein, but not to human MSLN epitope cluster E2 (SEQ ID NO: 1176, aa 247-384 position according to Kabat), E3 (SEQ ID NO: 1177, aa 385-453 position according to Kabat), E4 (SEQ ID NO: 1178, aa 454-501 position according to Kabat) and/or E5 (SEQ ID NO: 1179 aa 502-545 position according to Kabat), or wherein the first target cell surface antigen and/or the second target cell surface antigen is human CDH3 (SEQ ID NOs: 1170), and wherein the first and/or third binding domain of the antigen-binding molecule of claim 1 binds to human CDH3 epitope cluster D2B (SEQ ID NO: 1171, aa 253-290 position according to Kabat), D2C (SEQ ID NO: 1172 aa 291-327 position according to Kabat), D3A (SEQ ID NO: 1173 aa 328-363 position according to Kabat) and D4B (SEQ ID NO: 1174, aa 476-511 position according to Kabat) as determined by murine chimere sequence analysis.

28. (canceled)

29. The antigen-binding molecule of claim 1, wherein the second binding domain and the fourth binding domain both have an affinity lower than characterized by a KD value of about 1.2×10-8 M measured by surface plasmon resonance (SPR), or an affinity characterized by a KD value of about 1.2×10-8 M measured by SPR.

30. The antigen-binding molecule of claim 1, wherein the second binding domain and the fourth binding domain have an affinity characterized by a KD value of about 1.0×10-7 to 5.0×10-6 M measured by SPR.

31. (canceled)

32. The antigen-binding molecule of claim 1, wherein each of the second and the fourth binding domain (CD3 binding domains) individually has an at least about 10-fold lower activity than one CD3 binding domain comprising a VH according to SEQ ID NO 43 and a VL according to SEQ ID NO 44 (i.e. in a mono targeting context in contrast to a dual targeting context).

33. The antigen-binding molecule of claim 1, wherein the second and the fourth binding domain comprise a VH region comprising CDR-H 1, CDR-H2 and CDR-H3 selected from SEQ ID NOs 37 to 39, 45 to 47, 53 to 55, 61 to 63, 69 to 71, 436 to 438, 1126 to 1128, 1136 to 1138, 1142 to 1144, and 1148 to 1150, and a VL region comprising CDR-L1, CDR-L2 and CDR-L3 selected from SEQ ID NOs 40 to 42, 48 to 50, 56 to 58, 64 to 66, 72 to 74, 439 to 441, 1129 to 1131, 1139 to 1141, 1145 to 1147, and 1151 to 1153.

34. The antigen-binding molecule of claim 1, wherein the second and fourth binding domain comprise a VH region selected from SEQ ID NOs 43, 51, 59, 67, 75, 442 and 1132.

35. The antigen-binding molecule of claim 1, wherein the second and fourth binding domain comprise a VL region selected from SEQ ID NOs 44, 52, 60, 68, 76, 443 and 1133.

36. The antigen-binding molecule of claim 1, wherein the second binding domain and fourth binding domain comprising a VH region selected from SEQ ID NOs 43, 51, 59, 67, 75, 442 and 1132, and a VL region selected from SEQ ID NOs 44, 52, 60, 68, and 76, 443 and 1133, preferably 68, wherein when the VH region is 1132 and the VL region is 1133, the second binding domain and/or fourth binding domain as scFab domain additionally comprises a CH1 domain of SEQ ID NO: 1134 and a CLK domain of SEQ ID NO: 1135, and wherein the VH and VL region are linked to each other by a linker selected from SEQ ID NO 1, and 3 and 1125.

37. The antigen-binding molecule of claim 1, wherein the first binding domain and/or the third binding domain bind to CDH3 and comprise a VH region comprising SEQ ID NO: 1154 as CDR-H 1 wherein X1 is S or N, X2 is Y or S, X3 is P or W, X4 is I or M and X5 is Y, N or H; SEQ ID NO: 1155 as CDR-H2 wherein X1 is K, V, N or R; X2 is A, D, R, Y, S, W or H; X3 is Y, S, P, G or T; X4 is S, G or K; X5 is A, V, D, K, G, or T; X6 is A, V, D, K, S, G or H; X7 is Y, G, or E; X8 is K, I, or N; X9 is A, S, or N; X10 is S, Q or G; X11 is S or K; X12 is F or V; and X13 is K or Q; and SEQ ID NO: 1156 as CDR-H3, wherein X1 is F or Q; X2 is R,K,S or W; X3 is G or D; X4 is Y, P or R; X5 is R, S, G, N or T; X6 is Y, A or H; X7 is F, L or M; X8 is A or V; and X9 is Y or V; and wherein the first binding domain and/or the third (target) binding domain bind to CDH3 and comprise a VL region comprising SEQ ID NO: 1158 as CDR-L 1 wherein X1 is K or R, X2 is A or S; X3 is Q,D,S,G or E; X4 is S, D or N; X5 is V, L or I; X6 is, K, Y, S, or H; X7 is S or N; X8 is F, L or M; and X9 is A,N or H; SEQ ID NO: 1159 as CDR-L 2 wherein X1 is Y, G, W, or N; X2 is T or A; X3 is S or K; X4 is T, N or R; X5 is L or R; X6 is E, A, V or H; and X7 is S or E; and SEQ ID NO: 1160 as CDR-L3 wherein X1 is Q or V; X2 is Q, N or H; X3 is F, L, Y, W, N, or H; X4 is A, D, Y, S or N; X5 is Q, R, S, G, W or M; X6 is T, Y or F; and X7 is F, Y or L.

38. The antigen-binding molecule of claim 1, wherein the first binding domain and/or the third (target) binding domain bind to MSLN and comprise a VH region comprising SEQ ID NO: 1162 as CDR-H 1 wherein X1 is S, G or D; X2 is Y, A, G or F; X3 is I, W, or M; and X4 is V, S, G, T, or H; SEQ ID NO: 1163 as CDR-H 2 wherein X1 is A, S, N, W, Y, or V; X2 is Y, S or N; X3 is Y, G, P, or S; X4 is D, H, S, or N; X5 is G or S; X6 is E, G or S; X7 is G, S, N, F, T or Q; X8 is S, W, K, D, I or T; X9 is Y or N; X10 is A or N; X11 is A, P, N, D, E, I or Q; X12 is D, A, S or K; X13 is V, L, or F; X14 is K or Q; and X15 is G or S; and SEQ ID NO: 1164 as CDR-H 3 wherein X1 is D, E or V; X2 is R, G, or E; X3 is Y, A, or N; X4 is S,Y,V, or H; X5 is A,P,F, Y, or H; X6 is R or S; X7 is E or G; X8 is Y or L; X9 is R, Y or L; X10 is Y or G; X11 is D or Y; X12 is R, Y, or F; X13 is M,S,F,D or Y; X14 is A, G, S, or T; X15 is L, M, or F; and X16 is Y, I or V; and wherein the first binding domain and/or the third binding domain bind to MSLN and comprise a VL region comprising SEQ ID NO: 1166 as CDR-L 1 wherein X1 is A or S; X2 is G or S; X3 is E or Q; X4 is G,S or K; X5 is I, L, V or F; X6 is R,G or S; X7 is D,S,N or T; X8 is A,S,K or T; X9 is Y or W; X10 is V or L; and X11 is Y or A; SEQ ID NO 1167 as CDR-L2 wherein X1 is A,G or Q; X2 is A or S; X3 is S or T; X4 is G, S, K, I or T; X5 is R or L; X6 is A, P or Q; and X7 is S or T; and SEQ ID NO 1168 as CDR-L 3 wherein X1 is A or Q; X2 is Y, S, A, or T; X3 is G, E, Y, H or Q; X4 is A or S; X5 is S, T or F; X6 is P or T; X7 is R, A, L or F; and X8 is V or T.

39. The antigen-binding molecule of claim 1, wherein the first binding domain and/or the third binding domain bind to CDH3 and comprise a VH region of SEQ ID NO: 1157 wherein X1 is Q or E; X2 is V,L; X3 is Q,E; X4 is A or G; X5 is G or E; X6 is V or L; X7 is K or V X8 is K or Q, X9 is A or G, X10 is V or L, X11 is K or R, X12 is V or L, X13 is A or K, X14 is Y or F, X15 is T or S, X16 is T or S, X17 is S or N, X18 is Y or S, X19 is P or W, X20 is I or M, X21 is Y, N or H, X22 is T or A, X23 is Q or K, X24 is V or M, X25 is S or G, X26 is K, V, N or R, X27 is A, D, R, Y, S, W or H, X28 is Y, S, P, Gr or T, X29 is S, K, or G, X30 is A, V, D, K, or, T; X31 is A, D, K, S, G, or H; X32 is Y,G, or E, X33 is K, I, or N, X34 is A,S, or N, X35 is S, Q, or G, X36 is S or K, X37 is F or V, X38 is Q or K, X39 is F or V, X40 is I or M, X41 is T or S, X42 is V,I or R, X43 is T, K or N, X44 is T, A, S or K, X45 is S or N, X46 is A, V or L, X47 is L or M, X48 is Q or E, X49 is L or M, X50 is S or N, X51 is S or R, X52 is T or R, X53 is A or S, X54 is G, D or E; X55 is T or S, X56 is T, K, or R, X57 is S, Q, W, or R, X58 is D, or G, X59 is Y, P, or R; X60 is F,S,G,N or T, X61 is Y, A, or H, X62 is A,-, or V, X63 is F or M, X64 is Y or V; X65 is T,L or M; and a VL region of SEQ ID NO 1161, wherein X1 is D or E; X2 is Q or V; X3 is L, M; X4 is A,S or D; X5 is F,S or T; X6 is A or S; X7 is A or V; X8 is P,V or L; X9 is D or E; X10 is A or V; X11 is I or L; X12 is T, S, or N; X13 is K or R; X14 is A, S; or X15 is Q,D,S,G or E; X16 is S, D or N; X17 is V, I or L; X18 is K, Y, S or H; X19 is S or N; X20 is F, L or M; X21 is A, N or H; X22 is K or Q; X23 is A, P or V; X24 is K or R; X25 is I or V; X26 is Y, G, W or N; X27 is T or A; X28 is S or K; X29 is T, N or R; X30 is L or R; X31 is E, A, V or H; X32 is S or E; X33 is A, S, V or D; X34 is D or E; X35 is T or K; X36 is S or R; X37 is A,S or P; X38 is F or V; X39 is A,G; X40 is T or V; X41 is Q or V; X42 is Q, N, H; X43 is F, L, Y, W, N or H; X44 is A, D, Y, S or N; X45 is Q, R, S, G, W or M; X46 is F, Y or T; X47 is F, Y or L; X48 is V or L; and X49 is D or E.

40. The antigen-binding molecule of claim 1, wherein the first binding domain and/or the third binding domain bind to MSLN and comprise a VH region of SEQ ID NO: 1165, X1 is E or Q; X2 is V, L or Q; X3 is E or Q; X4 is A,G or P; X5 is E or G; X6 is V or L; X7 is V or K; X8 is K or Q; X9 is G or S; X10 is E, A, G or R; X11 is S or T; X12 is V or L; X13 is R, S or K; X14 is V or L; X15 is S or T; X16 is A,K or T; X17 is A or V; X18 is Y, I or F; X19 is S or T; X20 is S or F; X21 is S or T; X22 is D, G or S; X23 is Y, G, A or F; X24 is I, W or M; X25 is G, S, V, T or H; X26 is I or V; X27 is A or P; X28 is M, K or Q; X29 is G or C; X30 is I, M, V or L; X31 is A, G or S; X32 is A, S, N, W, Y or V; X33 is Y, S or N; X34 is Y, G, P or S; X35 is D, H, S or N; X36 is G or S; X37 is E, G or S; X38 is G, S, N, F, T or Q; X39 is S, K, W, D, I, or T; X40 is Y or N; X41 is A or N; X42 is A, P, N, E, D, I or Q; X43 is D, A, S or K; X44 is V,L or F; X45 is K,Q; X46 is G or S; X47 is V or F; X48 is I or M; X49 is S or T; X50 is R or V; X51 is N or T; X52 is A or S; X53 is I or K; X54 is S or N; X55 is S, T or Q; X56 is A, L or F; X57 is Y, S or F; X58 is L or M; X59 is E, K or Q; X60 is M or L; X61 is S or N; X62 is R or S; X63 is V or L; X64 is R or T; X65 is A or S; X66 is D, A or E; X67 is R or K; X68 is D, E, V or L; X69 is E, R, G or P; X70 is R, A, Nor Y; X71 is G, S, Y, V or H; X72 is A, P, F, D or Y; X73 is R or G; X74 is M, R, S or D; X75 is E or G; X76 is Y or L; X77 is Y or F; X78 is Y, S or F; X79 is A, G, S, T or H; X80 is L, M or F; X81 is Y, I or V; and X82 is L, M or T; and a VL region of SEQ ID NO 1169 X1 is E,S or D; X2 is Y,I or L; X3 is E, V or T; X4 is V,L or M; X5 is P or S; X6 is G or S; X7 is S or T; X8 is V or L; X9 is A, V or L; X10 is P or V; X11 is E, Q or D; X12 is R or T; X13 is A or V; X14 is S or T; X15 is I or L; X16 is S or T; X17 is A or S; X18 is G or S; X19 is E or Q; X20 is G,S or K; X21 is I, V, L or F; X22 is R, G or S; X23 is D or S; X24 is A, S, N, K or T; X25 is Y, W or M; X26 is V or L; X27 is Y or A; X28 is K or Q; X29 is A,S or V; X30 is R,V or K; X31 is V or L; X32 is A,G or Q; X33 is A or S; X34 is S or T; X35 is G,S,K,I or T; X36 is R or L; X37 is A, P or Q; X38 is S or T; X39 is I or V; X40 is E,S or D; X41 is G or N; X42 is N or T; X43 is D or T; X44 is A or F; X45 is R,G or S; X46 is L or T; X47 is E or Q; X48 is A or P; X49 is E or M; X50 is E or F; X51 is D, V or T; X52 is A or Q; X53 is Y,S,A or T; X54 is G,E, Y,H or Q; X55 is A or S; X56 is S,T or F; X57 is P or T; X58 is R, A, L or F; X59 is V or T; X60 is P or C; X61 is V or L; X62 is E or T; X63 is I or V; and X64 is L or K.

41. The antigen-binding molecule of claim 1, wherein the first and/or the third (target) binding domain comprise a VH region comprising CDR-H 1, CDR-H2 and CDR-H3 selected from SEQ ID NO: 77 to 79, 86 to 88, 95 to 97, 103 to 105, 111 to 113, 119 to 121, 127 to 129, 135 to 137, 143 to 145, 151 to 153, 159 to 161, 168 to 170, 177 to 179, 185 to 187, 194 to 196, 203 to 205, 212 to 214, 221 to 223, 230 to 232, 238 to 240, 334 to 336, 356 to 358, 365 to 367, 376 to 378, 385 to 387 and 194, 432 and 196, 446 to 448, 454 to 456, 462 to 464, 470 to 472, 478 to 480, 486 to 488, 494 to 496, 502 to 504, 510 to 512, 518 to 520, 526 to 528, 534 to 536, 542 to 544, 550 to 552, 558 to 560, 566 to 568, 574 to 576, 582 to 584, 590 to 592, 598 to 600, 606 to 608, 614 to 616, 622 to 624, 630 to 632, 638 to 640, 646 to 648, 654 to 656, 662 to 664, 670 to 672, 678 to 680, 686 to 688, 694 to 696, 702 to 704, 710 to 712, 718 to 720, 726 to 728, 734 to 736, 742 to 744, 750 to 752, 758 to 760, 766 to 768, 774 to 776, 782 to 784, 790 to 792, 798 to 800, 806 to 808, 814 to 816, 822 to 826, 830 to 832, 838 to 840, 846 to 848, 854 to 856, 862 to 864, 870 to 872, 878 to 880, 886 to 888, 894 to 896, 902 to 904, 910 to 912, 918 to 920, 926 to 928, 934 to 936, 942 to 944, 950 to 952, 958 to 960, 966 to 968, 974 to 976, 982 to 984, 990 to 992, 998 to 1000, 1006 to 1008, 1014 to 1016, 1022 to 1024, 1030 to 1032, 1038 to 1040, 1046 to 1048, 1054 to 1056, and 1062 to 1064, or any combination of CDR-H 1, CDR-H2 and CDR-H3 as disclosed together in Table 52.

42. The antigen-binding molecule of claim 1, wherein the first binding domain and/or third binding domain comprise a VL region comprising CDR-L1, CDR-L2 and CDR-L3 selected from SEQ ID NO: 80 to 82, 89 to 91, 98 to 100, 106 to 108, 114 to 116, 122 to 124, 130 to 132, 138 to 140, 146 to 148, 154 to 156, 162 to 164, 171 to 173, 180 to 182, 188 to 190, 197 to 199, 206 to 208, 215 to 217, 224 to 226, 233 to 235, 241 to 243, 337 to 339, 359 to 361, 368 to 370, 379 to 381, 388 to 390, 449 to 451, 457 to 459, 465 to 467, 473 to 475, 481 to 483, 489 to 491, 497 to 499, 505 to 507, 513 to 515, 521 to 523, 529 to 531, 537 to 539, 545 to 547, 553 to 555, 561 to 563, 569 to 571, 577 to 579, 585 to 587, 593 to 595, 601 to 603, 609 to 611, 617 to 619, 625 to 627, 633 to 635, 641 to 643, 649 to 651, 657 to 659, 665 to 667, 673 to 675, 681 to 683, 689 to 691, 697 to 699, 705 to 707, 713 to 715, 721 to 723, 729 to 731, 737 to 739, 745 to 747, 753 to 755, 761 to 763, 769 to 771, 777 to 779, 785 to 787, 793 to 795, 801 to 803, 809 to 811, 817 to 819, 825 to 829, 833 to 835, 841 to 843, 849 to 851, 857 to 859, 865 to 867, 873 to 875, 881 to 883, 889 to 891, 897 to 899, 905 to 907, 913 to 915, 921 to 923, 929 to 931, 937 to 939, 945 to 947, 953 to 955, 961 to 963, 969 to 971, 977 to 979, 985 to 987, 993 to 995, 1001 to 1003, 1009 to 1011, 1017 to 1019, 1025 to 1027, 1033 to 1035, 1041 to 1043, 1049 to 1051, 1057 to 1059, and 1065 to 1067 or any combination of CDR-L 1, CDR-L2 and CDR-L3 as disclosed together in the sequence table Tab. 52.

43. The antigen-binding molecule of claim 1, wherein the first binding domain and/or third binding domain comprise a VH region selected from SEQ ID NO: 83, 92, 101, 109, 117, 125, 133, 141, 149, 157, 165, 174, 183, 191, 200, 209, 218, 227, 236, 244, 340, 362, 371, 382, 391, and 433, 452, 460, 468, 476, 484, 492, 500, 508, 516, 524, 532, 540, 548, 556, 564, 572, 580, 588, 596, 604, 612, 620, 628, 636, 644, 652, 660, 668, 676, 684, 692, 700, 708, 716, 724, 732, 740, 748, 756, 764, 772, 780, 788, 796, 804, 812, 820, 828, 836, 844, 852, 860, 868, 876, 884, 892, 900, 908, 916, 924, 932, 940, 948, 956, 964, 972, 980, 988, 996, 1004, 1012, 1020, 1028, 1036, 1044, 1052, 1060, and 1068 or any VH as disclosed together in Table 52.

44. The antigen-binding molecule of claim 1, wherein the first binding domain and/or third binding domain comprises a VL region selected from SEQ ID NO: 84, 93, 102, 110, 118, 126, 134, 142, 150, 158, 166, 175, 184, 192, 201, 210, 219, 228, 237, 245, 341, 363, 372, 383, 392, 453, 461, 469, 477, 485, 493, 501, 509, 517, 525, 533, 541, 549, 557, 565, 573, 581, 589, 597, 605, 613, 621, 629, 637, 645, 653, 661, 669, 677, 685, 693, 701, 709, 717, 725, 733, 741, 749, 757, 765, 773, 781, 789, 797, 805, 813, 821, 829, 837, 845, 853, 861, 869, 877, 885, 893, 901, 909, 917, 925, 933, 941, 949, 957, 965, 973, 981, 989, 997, 1005, 1013, 1021, 1029, 1037, 1045, 1053, 1061, and 1069 or any VL as disclosed together in Table 52.

45. The antigen-binding molecule of claim 1, wherein the first binding domain and/or third binding domain comprises a VL region of increased stability by a single amino acid exchange (E to I), selected from SEQ ID NO: 85, 94, 193, 202, 211, 220, 229, 364, 384, 393.

46. The antigen-binding molecule of claim 1 comprising an amino acid sequence selected from the group consisting of SEQ ID NOs: 246 to 323, 330 to 332, 351 to 355, 373 to 375, 394 to 410, 434, 1073, 1075 to 1080, or any other full length multitargeting bispecific antigen-binding molecule as disclosed in Table 52.

47. A polynucleotide encoding the antigen-binding molecule of claim 1.

48. A vector comprising the polynucleotide of claim 47.

49. A host cell transformed or transfected with the polynucleotide of claim 47.

50. A process for producing an antigen-binding molecule, said process comprising culturing the host cell of claim 49 under conditions allowing the expression of the antigen-binding molecule and recovering the antigen-binding molecule from the culture.

51. A pharmaceutical composition comprising the antigen-binding molecule of claim 1.

52-54. (canceled)

55. A method for treating or ameliorating a proliferative disease, a tumorous disease, a cancer or an immunological disorder, the method comprising administering to a subject in need thereof the antigen binding molecule of claim 1.

56-57. (canceled)

58. The method of claim 55, wherein the disease is acute myeloid leukemia (AML), non-Hodgkin lymphoma (NHL), non-small-cell lung carcinoma (NSCLC), pancreatic cancer or colorectal cancer (CRC).

59. (canceled)

60. A kit comprising the antigen-binding molecule of claim 1 and instructions for use.
